(19) United States
(12) Patent Application Publication
(10) Pub. No.: US 2026/0324281 A1
(43) Pub. Date: Oct. 15, 2026

(54) HETEROCYCLIC COMPOUND AND ORGANIC LIGHT-EMITTING DEVICE COMPRISING SAME

(71) Applicant: LT MATERIALS CO., LTD., Yongin-si (KR)

(72) Inventors: Kyu-Tae SEO, Yongin-si (KR); Hyun-Joo LEE, Yongin-si (KR); Young-Seok NO, Yongin-si (KR); Dong-Jun KIM, Yongin-si (KR); Dae-Hyuk CHOI, Yongin-si (KR)

(21) Appl. No.: 19/679,619

(22) Filed: May 16, 2026

Related U.S. Application Data
(63) Continuation of application No. PCT/KR2024/017791, filed on Nov. 12, 2024.

(30) Foreign Application Priority Data
Nov. 16, 2023 (KR) 10-2023-0159566

(51) Int. Cl. H10K 85/60; C07B 59/00; C07D 403/14; C07D 405/14; C07D 409/14; C09K 11/02; H10K 50/11

(52) U.S. Cl. CPC H10K 85/654; C07B 59/002; C07D 403/14; C07D 405/14; C07D 409/14; C09K 11/02; H10K 85/615; H10K 85/622; H10K 85/6572; H10K 85/6574; H10K 85/6576; H10K 50/11

(57) ABSTRACT

Provided are a heterocyclic compound and an organic light-emitting device including the heterocyclic compound. The heterocyclic compound can be used as a material for an organic material layer of an organic light-emitting device. Specifically, it can serve as a material for a light-emitting layer and the like in the organic light-emitting device. More specifically, when used in the organic material layer of the organic light-emitting device, significantly excellent effects in driving, efficiency, and lifespan of the device can be obtained.

### TECHNICAL FIELD

The present invention relates to a heterocyclic compound and an organic light-emitting device comprising the same.

This application claims the benefit of priority to Korean Patent Application No. 10-2023-0159566, filed with the Korean Intellectual Property Office on Nov. 16, 2023, the entire contents of which are incorporated herein by reference.

### BACKGROUND ART

An electroluminescent device is a type of self-emissive display device that has advantages of a wide viewing angle, excellent contrast, and a fast response speed.

An organic light-emitting device has a structure in which an organic thin film is disposed between two electrodes. When a voltage is applied to the organic light-emitting device having such a structure, electrons and holes injected from the two electrodes combine in the organic thin film to form pairs, and then annihilate to emit light. The organic thin film may be composed of a single layer or multiple layers as necessary.

A material of the organic thin film may have a light-emitting function as necessary. For example, as the material of the organic thin film, a compound capable of independently forming a light-emitting layer by itself may be used, or a compound capable of acting as a host or a dopant of a host-dopant-based light-emitting layer may be used. In addition, as the material of the organic thin film, a compound capable of performing functions such as hole injection, hole transport, electron blocking, hole blocking, electron transport, and electron injection may be used.

In order to improve performance, efficiency, and a lifespan of the organic light-emitting device, the development of the material for the organic thin film is continuously required.

### PRIOR ART DOCUMENTS

### Patent Document

(Patent Document 1) U.S. Pat. No. 4,356,429

### SUMMARY OF THE INVENTION

### Technical Problem

An object of the present invention is to provide a heterocyclic compound and an organic light-emitting device comprising the same.

### Solution to the Problem

An exemplary embodiment of the present application provides a heterocyclic compound represented by the following Chemical Formula 1.

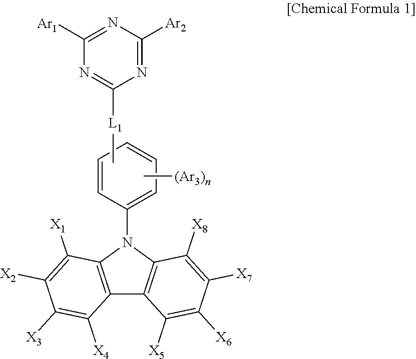

In Chemical Formula 1,
Ar₁ and Ar₂ are the same as or different from each other, and are each independently a substituted or unsubstituted C6 to C60 aryl group; or a substituted or unsubstituted C2 to C60 heteroaryl group;
L₁ is a direct bond; or a substituted or unsubstituted C6 to C60 arylene group;
Ar₃ is hydrogen; deuterium; a substituted or unsubstituted C6 to C60 aryl group; or a substituted or unsubstituted C2 to C60 heteroaryl group,
n is an integer from 1 to 4, and when n is 2 or greater, substituents in parentheses are the same as or different from each other;
when L₁ is a direct bond, Ar₃ is a substituted or unsubstituted C6 to C60 aryl group; or a substituted or unsubstituted C2 to C60 heteroaryl group;
X₁ to X₅ and X₇ are the same as or different from each other, and are each independently hydrogen; deuterium; or a substituted or unsubstituted C6 to C60 aryl group;
any one of X₆ and X₈ is represented by the following Chemical Formula 2, and the remainder is hydrogen; or deuterium;

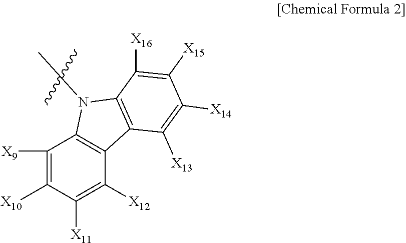

X₉ to X₁₆ are the same as or different from each other, and are each independently hydrogen; deuterium; or a substituted or unsubstituted C6 to C60 aryl group;

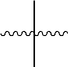

represents a site bonded to X₆ or X₈ of Chemical Formula 1; and
at least six of X₁ to X₁₆ are substituted with deuterium.

In addition, an exemplary embodiment of the present application provides an organic light-emitting device comprising: a first electrode; a second electrode provided to face the first electrode; and one or more organic material layers provided between the first electrode and the second electrode, wherein one or more layers of the organic material layers comprise the heterocyclic compound represented by Chemical Formula 1 described above.

### Advantageous Effects of the Invention

The heterocyclic compound described in the present specification can be used as a material for an organic material layer of an organic light-emitting device. Specifically, it can serve as a material for a light-emitting layer and the like in the organic light-emitting device.

More specifically, when used in the organic material layer of the organic light-emitting device, significantly excellent effects in driving, efficiency, and lifespan of the device can be obtained.

### BRIEF DESCRIPTION OF DRAWINGS

FIGS. 1 to 3 are diagrams schematically illustrating a stacked structure of an organic light-emitting device according to an exemplary embodiment of the present application, respectively.

### DETAILED DESCRIPTION OF THE INVENTION

Hereinafter, the present specification will be described in more detail.

In the present specification, when a certain part “comprises” or “includes” a certain component, this means that other components are not excluded, but other components can be further included unless otherwise specifically stated.

In the present specification,

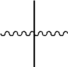

in the chemical formula represents a bonding position.

In the present specification, n in Cn means the number of carbon atoms. That is, for example,

C6 to C60 means 6 to 60 carbon atoms.

In the present specification, the term “substitution” means that a hydrogen atom bonded to a carbon atom of a compound is replaced with another substituent, and a position to be substituted is not limited as long as it is a position where the hydrogen atom is substituted, that is, a position where the substituent can be substituted, and when two or more are substituted, two or more substituents may be the same as or different from each other.

In the present specification, “substituted or unsubstituted” means substituted or unsubstituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent in which two or more substituents selected from the above-exemplified substituents are connected, and R, R′, and R″ are each independently a substituent consisting of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “a case where a substituent is not indicated in a chemical formula or a compound structure” means that a hydrogen atom is bonded to a carbon atom. However, since deuterium (²H) is an isotope of hydrogen, some hydrogen atoms may be deuterium.

In an exemplary embodiment of the present application, “a case where a substituent is not indicated in a chemical formula or a compound structure” may mean that all positions at which a substituent may come are hydrogen or deuterium. That is, in the case of deuterium, it is an isotope of hydrogen, and some hydrogen atoms may be deuterium which is an isotope, and in this case, a content of deuterium may be 0% to 100%.

In an exemplary embodiment of the present application, in “a case where a substituent is not indicated in a chemical formula or a compound structure,” hydrogen and deuterium may be mixed and used in the compound when deuterium is not explicitly excluded, such as when the content of deuterium is 0%, the content of hydrogen is 100%, and all substituents are hydrogen.

In an exemplary embodiment of the present application, deuterium is one of the isotopes of hydrogen and is an element having a deuteron consisting of one proton and one neutron as a nucleus, which can be expressed as hydrogen-2, and an element symbol thereof may also be written as D or ²H.

In an exemplary embodiment of the present application, an isotope, which means an atom having the same atomic number (Z) but a different mass number (A), may also be interpreted as an element having the same number of protons but a different number of neutrons.

In an exemplary embodiment of the present application, the meaning of a content T % of a specific substituent may be defined as T2/T1×100=T % when the total number of substituents that a basic compound can have is defined as T1, and the number of specific substituents among them is defined as T2.

That is, in one example, a deuterium content of 20% in a phenyl group represented by

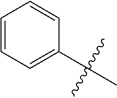 [SMILES: *c1ccccc1]

means that the total number of substituents that the phenyl group can have is 5 (T1 in the formula), and when the number of deuteriums among them is 1 (T2 in the formula), it can be expressed as 20%. That is, a 20% deuterium content in the phenyl group may be represented by the following structural formula.

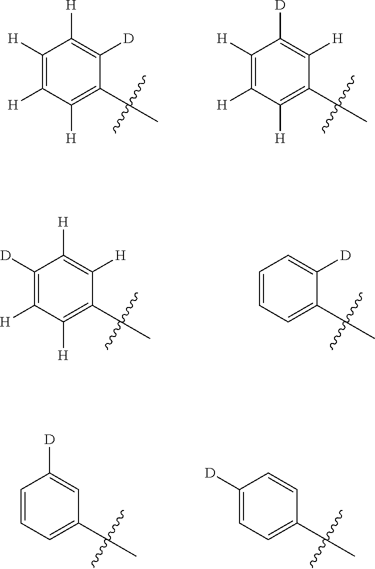 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in an exemplary embodiment of the present application, a “phenyl group having a deuterium content of 0%” may mean a phenyl group that does not contain a deuterium atom, that is, has five hydrogen atoms.

In the present specification, the halogen may be fluorine, chlorine, bromine, or iodine.

In the present specification, the alkyl group includes a straight or branched chain having 1 to 60 carbon atoms, and may be further substituted with another substituent. The number of carbon atoms in the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group includes a straight or branched chain having 2 to 60 carbon atoms, and may be further substituted with another substituent. The number of carbon atoms in the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, the alkynyl group includes a straight or branched chain having 2 to 60 carbon atoms, and may be further substituted with another substituent. The number of carbon atoms in the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, the alkoxy group is represented by —O(R101), and the examples of the aforementioned alkyl group may be applied to R101.

In the present specification, the aryloxy group is represented by —O(R102), and the examples of the aforementioned aryl group may be applied to R102.

In the present specification, the alkylthioxy group is represented by —S(R103), and the examples of the aforementioned alkyl group may be applied to R103.

In the present specification, the arylthioxy group is represented by —S(R104), and the examples of the aforementioned aryl group may be applied to R104.

In the present specification, the alkylsulfoxy group is represented by —S(═O)₂(R105), and the examples of the aforementioned alkyl group may be applied to R105.

In the present specification, the arylsulfoxy group is represented by —S(═O)₂(R106), and the examples of the aforementioned aryl group may be applied to R106.

In the present specification, the cycloalkyl group includes a monocyclic or polycyclic group having 3 to 60 carbon atoms, and may be further substituted with another substituent. Here, the polycyclic means a group in which a cycloalkyl group is directly connected to or condensed with another ring group. Here, the other ring group may be a cycloalkyl group, but may also be another type of ring group, such as a heterocycloalkyl group, an aryl group, a heteroaryl group, and the like. The number of carbon atoms in the cycloalkyl group may be 3 to 60, specifically 3 to 40, and more specifically 5 to 20. Specifically, examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group comprises O, S, Se, N, or Si as a heteroatom, includes a monocyclic or polycyclic group having 2 to 60 carbon atoms, and may be further substituted with another substituent. Here, the polycyclic means a group in which a heterocycloalkyl group is directly connected to or condensed with another ring group. Here, the other ring group may be a heterocycloalkyl group, but may also be another type of ring group, such as a cycloalkyl group, an aryl group, a heteroaryl group, and the like. The number of carbon atoms in the heterocycloalkyl group may be 2 to 60, specifically 2 to 40, and more specifically 3 to 20.

In the present specification, the aryl group includes a monocyclic or polycyclic group having 6 to 60 carbon atoms, and may be further substituted with another substituent. Here, the polycyclic means a group in which an aryl group is directly connected to or condensed with another ring group. Here, the other ring group may be an aryl group, but may also be another type of ring group, such as a cycloalkyl group, a heterocycloalkyl group, a heteroaryl group, and the like. The aryl group includes a spiro group. The number of carbon atoms in the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, condensed ring groups thereof, and the like, but are not limited thereto.

In the present specification, the terphenyl group may be selected from the following structures.

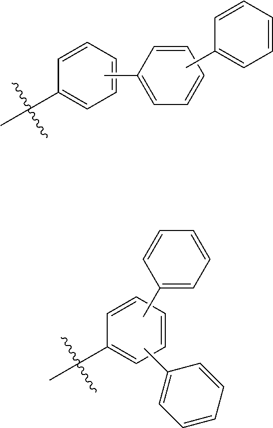

In the present specification, the fluorenyl group may be substituted, and adjacent substituents may bond to each other to form a ring.

When the fluorenyl group is substituted, it may be any one of the following structures, but is not limited thereto.

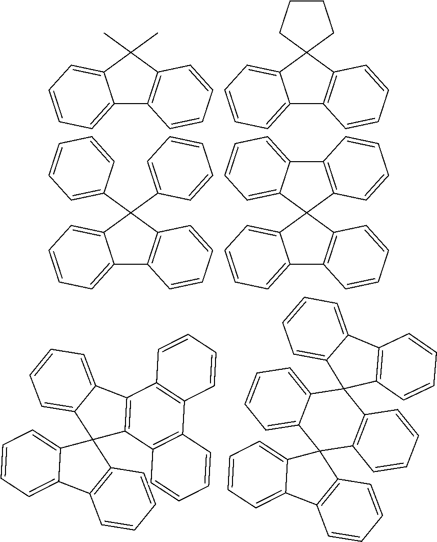

In the present specification, the heteroaryl group comprises S, O, Se, N, or Si as a heteroatom, includes a monocyclic or polycyclic group having 2 to 60 carbon atoms, and may be further substituted with another substituent. Here, the polycyclic means a group in which a heteroaryl group is directly connected to or condensed with another ring group. Here, the other ring group may be a heteroaryl group, but may also be another type of ring group, such as a cycloalkyl group, a heterocycloalkyl group, an aryl group, and the like. The number of carbon atoms in the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, a spirobi (dibenzosilole) group, a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, when a substituent is a carbazole group, it means bonding to nitrogen or carbon of the carbazole.

In the present specification, when the carbazole group is substituted, an additional substituent may be substituted on the nitrogen or carbon of the carbazole.

In the present specification, the benzocarbazole group may be any one of the following structures.

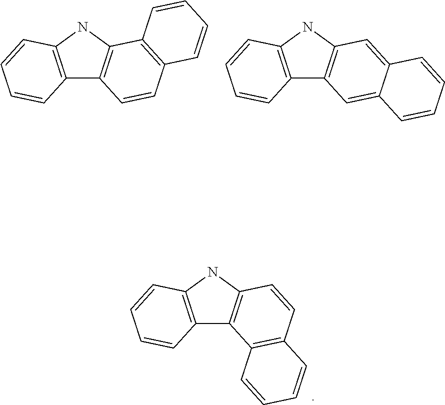

In the present specification, the dibenzocarbazole group may be any one of the following structures.

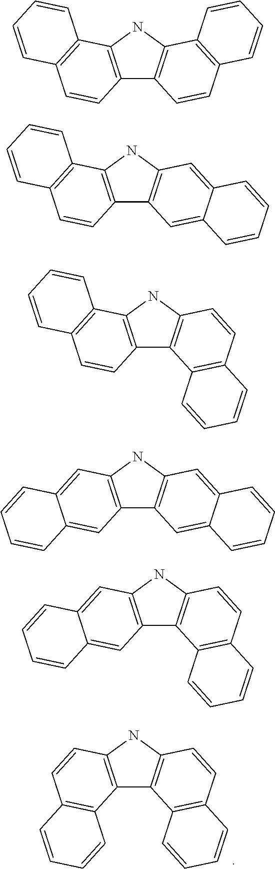

In the present specification, the naphthobenzofuran group may be any one of the following structures.

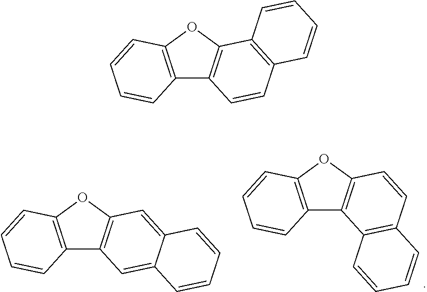

In the present specification, the naphthobenzothiophene group may be any one of the following structures.

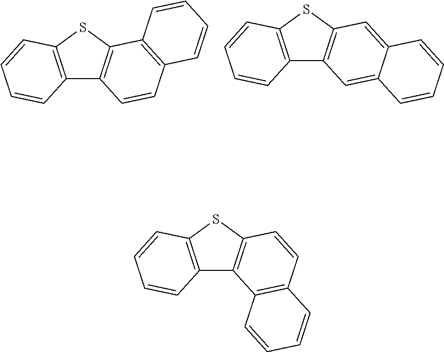

In the present specification, a silyl group is a substituent comprising Si and in which the Si atom is directly connected as a radical, and is represented by —Si(R107)(R108)(R109), and R107 to R109 are the same as or different from each other, and may each independently be a substituent consisting of at least one of hydrogen; deuterium; a halogen group; an alkyl group; an alkenyl group; an alkoxy group; a cycloalkyl group; a heterocycloalkyl group; an aryl group; and a heteroaryl group.

Specific examples of the silyl group include

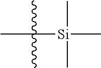 [SMILES: *[Si](C)(C)C]

a trimethylsilyl group,

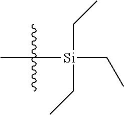 [SMILES: *[Si](CC)(CC)CC]

a triethylsilyl group,

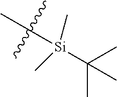 [SMILES: *[Si](C)(C)C(C)(C)C]

a t-butyldimethylsilyl group,

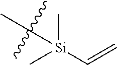 [SMILES: *[Si](C)(C)C=C]

a vinyldimethylsilyl group,

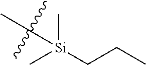 [SMILES: *[Si](C)(C)CCC]

a propyldimethylsilyl group,

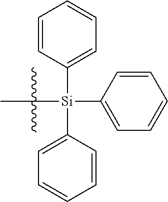 [SMILES: *[Si](c1ccccc1)(c1ccccc1)c1ccccc1]

a triphenylsilyl group,

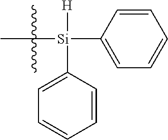 [SMILES: *[SiH](c1ccccc1)c1ccccc1]

a diphenylsilyl group,

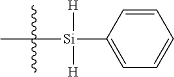 [SMILES: *[SiH2]c1ccccc1]

a phenylsilyl group, and the like, but are not limited thereto.

In the present specification, a phosphine oxide group is represented by —P(═O)(R110)(R111), and R110 and R111 are the same as or different from each other, and may each independently be a substituent consisting of at least one of hydrogen; deuterium; a halogen group; an alkyl group; an alkenyl group; an alkoxy group; a cycloalkyl group; a heterocycloalkyl group; an aryl group; and a heteroaryl group. Specifically, it may be substituted with an alkyl group or an aryl group, and the aforementioned examples may be applied to the alkyl group and the aryl group. For example, the phosphine oxide group includes a dimethylphosphine oxide group, a diphenylphosphine oxide group, a dinaphthylphosphine oxide group, and the like, but is not limited thereto.

In the present specification, an amine group is represented by —N(R112)(R113), and R112 and R113 are the same as or different from each other, and may each independently be a substituent consisting of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the examples of the aforementioned aryl group may be applied to an arylene group, except that the arylene group is a divalent group.

In the present specification, the examples of the aforementioned heteroaryl group may be applied to a heteroarylene group, except that the heteroarylene group is a divalent group.

In the present specification, an “adjacent” group may mean a substituent substituted on an atom directly connected to an atom on which the corresponding substituent is substituted, a substituent positioned sterically closest to the corresponding substituent, or another substituent substituted on the atom on which the corresponding substituent is substituted. For example, two substituents substituted at an ortho position in a benzene ring and two substituents substituted on the same carbon in an aliphatic ring may be interpreted as groups “adjacent” to each other.

A hydrocarbon ring and a heterocycle which may be formed by adjacent groups include an aliphatic hydrocarbon ring, an aromatic hydrocarbon ring, an aliphatic heterocycle, and an aromatic heterocycle, and the structures exemplified for the aforementioned cycloalkyl group, aryl group, heterocycloalkyl group, and heteroaryl group may be applied, respectively, except that the rings are not monovalent groups.

In an exemplary embodiment of the present application, all groups not indicated as substituents; or groups represented by hydrogen may mean that they are substitutable with deuterium. That is, hydrogen; or deuterium may indicate a state in which they are substitutable with each other.

In general, a compound bonded with hydrogen and a compound substituted with deuterium show a difference in thermodynamic behavior. This is because a mass of a deuterium atom is twice as large as that of hydrogen, and due to the difference in mass of the atoms, deuterium has a characteristic of having a lower vibrational energy.

In addition, a single bond dissociation energy between carbon and deuterium is higher than a single bond dissociation energy between carbon and hydrogen. Accordingly, a structure substituted with deuterium has an effect of improving a lifespan of a device using the same because thermal stability of a molecule increases.

When a compound is deposited on a silicon wafer, a material comprising deuterium tends to pack with a narrower intermolecular distance. In addition, when a surface of a thin film is observed with an atomic force microscope (AFM), it can be confirmed that the thin film manufactured with the compound comprising deuterium is deposited as a more uniform surface without an aggregated portion.

An exemplary embodiment of the present application provides a heterocyclic compound represented by the following Chemical Formula 1.

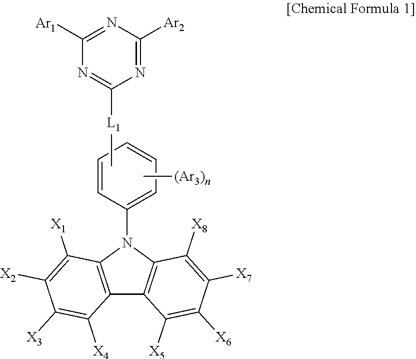

In Chemical Formula 1,
Ar₁ and Ar₂ are the same as or different from each other, and are each independently a substituted or unsubstituted C6 to C60 aryl group; or a substituted or unsubstituted C2 to C60 heteroaryl group;
L₁ is a direct bond; or a substituted or unsubstituted C6 to C60 arylene group;
Ar₃ is hydrogen; deuterium; a substituted or unsubstituted C6 to C60 aryl group; or a substituted or unsubstituted C2 to C60 heteroaryl group,
n is an integer from 1 to 4, and when n is 2 or greater, substituents in parentheses are the same as or different from each other;
when L₁ is a direct bond, Ar₃ is a substituted or unsubstituted C6 to C60 aryl group; or a substituted or unsubstituted C2 to C60 heteroaryl group;
X₁ to X₅ and X₇ are the same as or different from each other, and are each independently hydrogen; deuterium; or a substituted or unsubstituted C6 to C60 aryl group;
any one of X₆ and X₈ is represented by the following Chemical Formula 2, and the remainder is hydrogen; or deuterium;

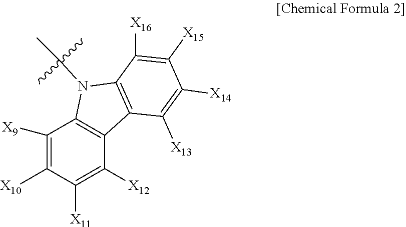

In Chemical Formula 2,
X₉ to X₁₆ are the same as or different from each other, and are each independently hydrogen; deuterium; or a substituted or unsubstituted C6 to C60 aryl group;

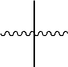

represents a site bonded to X₆ or X₈ of Chemical Formula 1; and
at least six of X₁ to X₁₆ are substituted with deuterium.

The heterocyclic compound represented by Chemical Formula 1 has a structure in which a carbazole of Chemical Formula 2 is bonded to the 1- or 3-position of a carbazole of Chemical Formula 1, at least six deuteriums are substituted in the two carbazoles, and at least one aryl group or heteroaryl group is bonded to a linker (phenyl-L1).

By having the above structure, two N-carbazole groups are bonded at a position (ortho, para) where resonance of unshared electron pairs is not broken, thereby enhancing hole transport capability, and phenyl-L1 introduced as a linker makes the molecular structure more robust to minimize energy canceled by rotation and vibration, thereby improving a lifespan and efficiency characteristics of a device, and a lifespan characteristic of the device may be significantly improved by substituting N-carbazole with deuterium.

In an exemplary embodiment of the present application, Chemical Formula 1 may be represented by any one of the following Chemical Formulas 1-1 to 1-3.

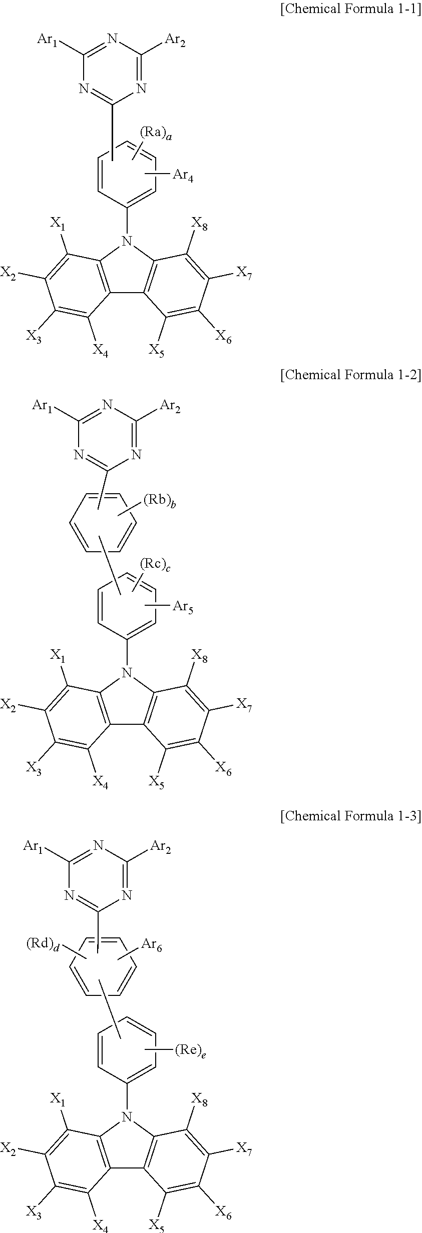

In Chemical Formulas 1-1 to 1-3,
definitions of Ar₁, Ar₂, and X₁ to X₈ are the same as the definitions in Chemical Formula 1;
Ar₄ to Ar₆ are each a substituted or unsubstituted C6 to C60 aryl group; or a substituted or unsubstituted C2 to C60 heteroaryl group; and
Ra, Rb, Rc, Rd, and Re are each independently hydrogen, deuterium, a substituted or unsubstituted C6 to C60 aryl group, or a substituted or unsubstituted C2 to C60 heteroaryl group;
a, c, and d are each an integer from 0 to 3, and when a, c, and d are each 2 or greater, substituents in parentheses are the same as or different from each other; and
b and e are each an integer from 0 to 4, and when b and e are each 2 or greater, substituents in parentheses are the same as or different from each other.

In an exemplary embodiment of the present application, Ar₁ and Ar₂ are the same as or different from each other, and may each independently be a substituted or unsubstituted C6 to C40 aryl group; or a substituted or unsubstituted C2 to C40 heteroaryl group.

In another exemplary embodiment, Ar₁ and Ar₂ are the same as or different from each other, and may each independently be a substituted or unsubstituted C6 to C20 aryl group; or a substituted or unsubstituted C2 to C20 heteroaryl group.

In another exemplary embodiment, Ar₁ and Ar₂ are the same as or different from each other, and may each independently be a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted dibenzofuran group; a substituted or unsubstituted dibenzothiophene group; or a substituted or unsubstituted carbazole group.

In another exemplary embodiment, Ar₁ and Ar₂ are the same as or different from each other, and may each independently be a substituted or unsubstituted phenyl group; a substituted or unsubstituted biphenyl group; a substituted or unsubstituted terphenyl group; a substituted or unsubstituted naphthyl group; or a substituted or unsubstituted fluorenyl group.

In another exemplary embodiment, Ar₁ and Ar₂ are the same as or different from each other, and may each independently be a phenyl group; a biphenyl group; a terphenyl group; or a naphthyl group.

In another exemplary embodiment, Ar₁ and Ar₂ may be phenyl groups.

In an exemplary embodiment of the present application, L₁ may be a direct bond; or a substituted or unsubstituted C6 to C40 arylene group.

In another exemplary embodiment, L₁ may be a direct bond; or a substituted or unsubstituted C6 to C20 arylene group.

In another exemplary embodiment, L₁ may be a direct bond; or a C6 to C20 arylene group substituted or unsubstituted with a C6 to C60 aryl group or a C2 to C60 heteroaryl group.

In another exemplary embodiment, L₁ may be a direct bond; or a phenylene group substituted or unsubstituted with a phenyl group, a dibenzofuran group, a dibenzothiophene group, or a carbazole group.

In an exemplary embodiment of the present application, Ar₃ may be hydrogen; deuterium; a substituted or unsubstituted C6 to C40 aryl group; or a substituted or unsubstituted C2 to C40 heteroaryl group.

In another exemplary embodiment, Ar₃ may be hydrogen; deuterium; a substituted or unsubstituted C6 to C20 aryl group; or a substituted or unsubstituted C2 to C20 heteroaryl group.

In another exemplary embodiment, Ar₃ may be hydrogen; deuterium; a substituted or unsubstituted phenyl group; a substituted or unsubstituted biphenyl group; a substituted or unsubstituted naphthyl group; a substituted or unsubstituted dibenzofuran group; a substituted or unsubstituted dibenzothiophene group; or a substituted or unsubstituted carbazole group.

In another exemplary embodiment, Ar₃ may be hydrogen; deuterium; a phenyl group; a biphenyl group; a naphthyl group; a dibenzofuran group; a dibenzothiophene group; or a carbazole group.

In another exemplary embodiment, Ar₃ may be hydrogen; deuterium; a phenyl group; a dibenzofuran group; or a dibenzothiophene group.

In an exemplary embodiment of the present application, when all of Ar₃ are hydrogen or deuterium, L₁ may be a C6 to C60 arylene group substituted with a C6 to C60 aryl group or a C2 to

C60 heteroaryl group.

In another exemplary embodiment, when all of Ar₃ are hydrogen or deuterium, L₁ may be a C6 to C20 arylene group substituted with a C6 to C60 aryl group or a C2 to C60 heteroaryl group.

In another exemplary embodiment, when all of Ar₃ are hydrogen or deuterium, L₁ may be a phenylene group substituted with a C6 to C60 aryl group or a C2 to C60 heteroaryl group.

In another exemplary embodiment, when all of Ar₃ are hydrogen or deuterium, L₁ may be a phenylene group substituted with a phenyl group, a dibenzofuran group, a dibenzothiophene group, or a carbazole group.

In an exemplary embodiment of the present application, n is an integer from 1 to 3, and when n is 2 or greater, substituents in parentheses may be the same as or different from each other.

In another exemplary embodiment, n is 1 or 2, and when n is 2, substituents in parentheses may be the same as or different from each other.

In another exemplary embodiment, n is 1.

In an exemplary embodiment of the present application, when L₁ is a direct bond, Ar₃ may be a substituted or unsubstituted C6 to C40 aryl group; or a substituted or unsubstituted C2 to C40 heteroaryl group.

In another exemplary embodiment, when L₁ is a direct bond, Ar₃ may be a substituted or unsubstituted C6 to C20 aryl group; or a substituted or unsubstituted C2 to C20 heteroaryl group.

In another exemplary embodiment, when L₁ is a direct bond, Ar₃ may be a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; or a substituted or unsubstituted dibenzothiophene group.

In another exemplary embodiment, when L₁ is a direct bond, Ar₃ may be a substituted or unsubstituted phenyl group; a substituted or unsubstituted biphenyl group; a substituted or unsubstituted dibenzofuran group; or a substituted or unsubstituted dibenzothiophene group.

In another exemplary embodiment, when L₁ is a direct bond, Ar₃ may be a phenyl group; a biphenyl group; a dibenzofuran group; or a dibenzothiophene group.

In an exemplary embodiment of the present application, Ra, Rb, Rc, Rd, and Re may each independently be hydrogen; deuterium; a substituted or unsubstituted C6 to C40 aryl group; or a substituted or unsubstituted C2 to C40 heteroaryl group.

In another exemplary embodiment, Ra, Rb, Rc, Rd, and Re may each independently be hydrogen; deuterium; a substituted or unsubstituted C6 to C20 aryl group; or a substituted or unsubstituted C2 to C20 heteroaryl group.

In another exemplary embodiment, Ra, Rb, Rc, Rd, and Re may each independently be hydrogen; deuterium; a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; or a substituted or unsubstituted dibenzothiophene group.

In another exemplary embodiment, Ra, Rb, Rc, Rd, and Re may each independently be hydrogen; or deuterium.

In another exemplary embodiment, Ra, Rb, Rc, Rd, and Re are each independently hydrogen.

In an exemplary embodiment of the present application, a, c, and d are each an integer from 0 to 2, and when a, c, and d are each 2, substituents in parentheses may be the same as or different from each other.

In another exemplary embodiment, a, c, and d are each 0 or 1.

In another exemplary embodiment, a, c, and d are each 1.

In another exemplary embodiment, a, c, and d are each 0.

In an exemplary embodiment of the present application, b and e are each an integer from 0 to 3, and when b and e are each 2 or greater, substituents in parentheses may be the same as or different from each other.

In another exemplary embodiment, b and e are each an integer from 0 to 2, and when b and e are each 2, substituents in parentheses may be the same as or different from each other.

In another exemplary embodiment, b and e are each 0 or 1.

In another exemplary embodiment, b and e are each 1.

In another exemplary embodiment, b and e are each 0.

In an exemplary embodiment of the present application, X₁ to X₅ and X₇ are the same as or different from each other, and may each independently be hydrogen; deuterium; or a substituted or unsubstituted C6 to C20 aryl group.

In another exemplary embodiment, X₁ to X₅ and X₇ are the same as or different from each other, and may each independently be hydrogen; deuterium; or a phenyl group substituted or unsubstituted with deuterium.

In another exemplary embodiment, X₃ is hydrogen; deuterium; or a phenyl group substituted or unsubstituted with deuterium, and X₁, X₂, X₄, X₅, and X₇ are the same as or different from each other, and may each independently be hydrogen; or deuterium.

In an exemplary embodiment of the present application, X₉ to X₁₆ are the same as or different from each other, and may each independently be hydrogen; deuterium; or a substituted or unsubstituted C6 to C20 aryl group.

In another exemplary embodiment, X₉ to X₁₆ are the same as or different from each other, and may each independently be hydrogen; deuterium; or a phenyl group substituted or unsubstituted with deuterium.

In another exemplary embodiment, X₉, X₁₁, X₁₄, and X₁₆ are the same as or different from each other, and may each independently be hydrogen; deuterium; or a phenyl group substituted or unsubstituted with deuterium, and X₁₀, X₁₂, X₁₃, and X₁₅ are the same as or different from each other, and may each independently be hydrogen; or deuterium.

In an exemplary embodiment of the present application, a deuterium content of the compound of Chemical Formula 1 may be 15% to 100%.

In an exemplary embodiment of the present application, a deuterium content of the compound of Chemical Formula 1 may be 30% to 100%.

In an exemplary embodiment of the present application, a deuterium content of the compound of Chemical Formula 1 may be 50% to 100%.

In an exemplary embodiment of the present application, a deuterium content of the compound of Chemical Formula 1 may be 70% to 100%.

In an exemplary embodiment of the present application, a deuterium content of the compound of Chemical Formula 1 may be 90% to 100%.

In an exemplary embodiment of the present application, at least seven of X₁ to X₁₆ may be substituted with deuterium.

In another exemplary embodiment, at least eight of X₁ to X₁₆ may be substituted with deuterium.

In another exemplary embodiment, at least nine of X₁ to X₁₆ may be substituted with deuterium.

In another exemplary embodiment, at least ten of X₁ to X₁₆ may be substituted with deuterium.

In another exemplary embodiment, at least eleven of X₁ to X₁₆ may be substituted with deuterium.

In another exemplary embodiment, at least twelve of X₁ to X₁₆ may be substituted with deuterium.

In another exemplary embodiment, at least thirteen of X₁ to X₁₆ may be substituted with deuterium.

In another exemplary embodiment, at least fourteen of X₁ to X₁₆ may be substituted with deuterium.

In another exemplary embodiment, X₁ to X₁₆ may all be substituted with deuterium.

In an exemplary embodiment of the present application, Chemical Formula 1 may be represented by any one of the following compounds.

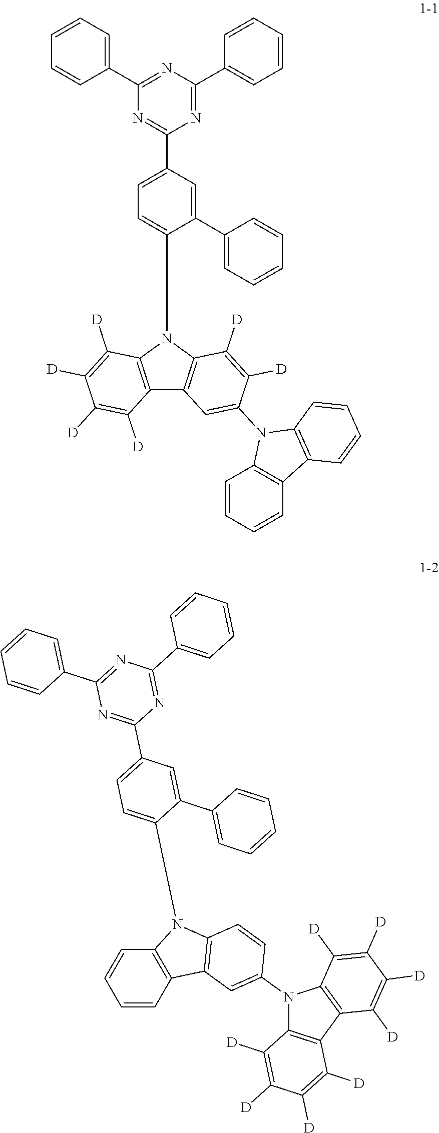 [SMILES: [2H]c1c([2H])c([2H])c2c(c1[2H])c1cc(-n3c4ccccc4c4ccccc43)c([2H])c([2H])c1n2-c1ccc(-c2nc(-c3ccccc3)nc(-c3ccccc3)n2)cc1-c1ccccc1 ; [2H]c1c([2H])c([2H])c2c(c1[2H])c1c([2H])c([2H])c([2H])c([2H])c1n2-c1ccc2c(c1)c1ccccc1n2-c1ccc(-c2nc(-c3ccccc3)nc(-c3ccccc3)n2)cc1-c1ccccc1]

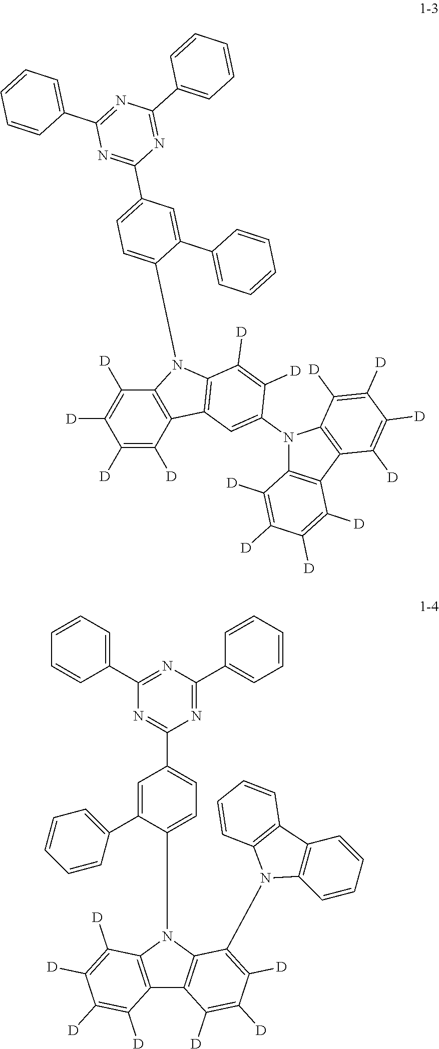 [SMILES: [2H]c1c([2H])c([2H])c2c(c1[2H])c1cc(-n3c4c([2H])c([2H])c([2H])c([2H])c4c4c([2H])c([2H])c([2H])c([2H])c43)c([2H])c([2H])c1n2-c1ccc(-c2nc(-c3ccccc3)nc(-c3ccccc3)n2)cc1-c1ccccc1 ; [2H]c1c([2H])c([2H])c2c(c1[2H])c1c([2H])c([2H])c([2H])c(-n3c4ccccc4c4ccccc43)c1n2-c1ccc(-c2nc(-c3ccccc3)nc(-c3ccccc3)n2)cc1-c1ccccc1]

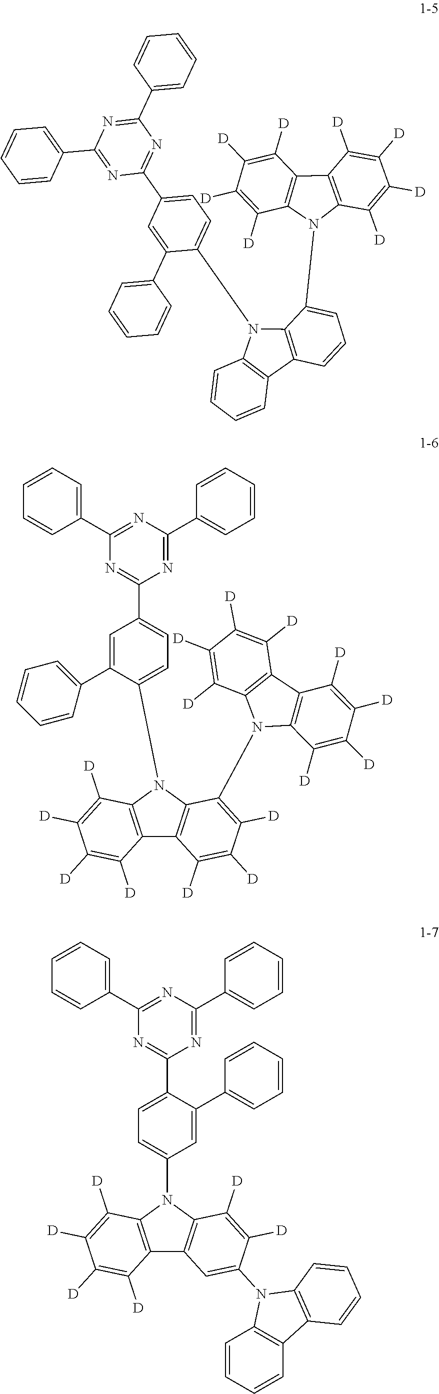 [SMILES: [2H]c1c([2H])c([2H])c2c(c1[2H])c1c([2H])c([2H])c([2H])c([2H])c1n2-c1cccc2c3ccccc3n(-c3ccc(-c4nc(-c5ccccc5)nc(-c5ccccc5)n4)cc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2nc(-c3ccccc3)nc(-c3ccccc3)n2)cc1-c1ccccc1 ; [2H]c1c([2H])c([2H])c2c(c1[2H])c1cc(-n3c4ccccc4c4ccccc43)c([2H])c([2H])c1n2-c1ccc(-c2nc(-c3ccccc3)nc(-c3ccccc3)n2)c(-c2ccccc2)c1]

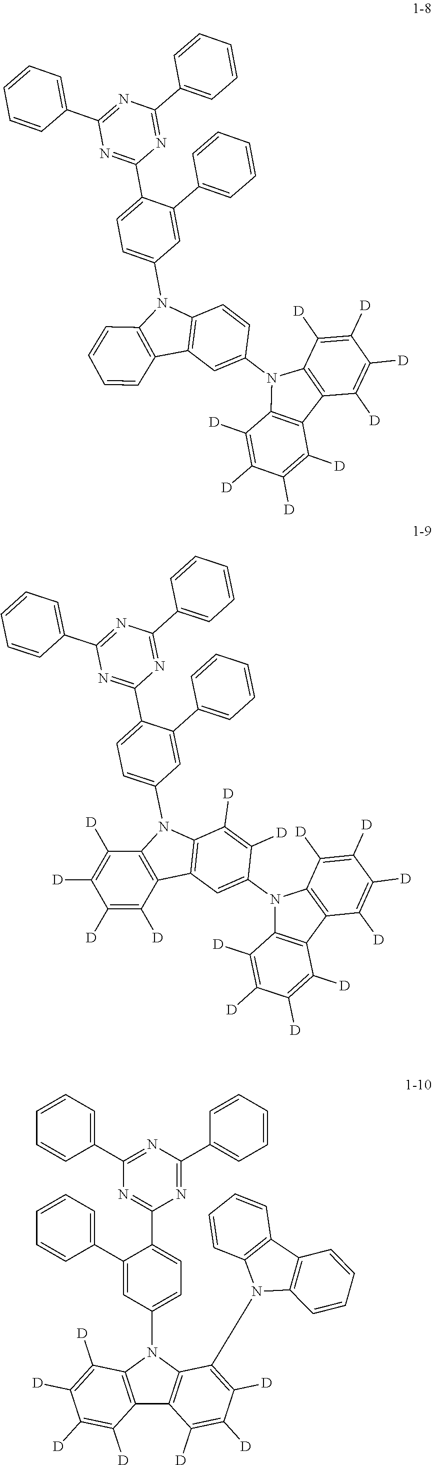 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2nc(-c3ccccc3)nc(-c3ccccc3)n2)c(-c2ccccc2)c1 ; [2H]c1c([2H])c([2H])c2c(c1[2H])c1cc(-n3c4c([2H])c([2H])c([2H])c([2H])c4c4c([2H])c([2H])c([2H])c([2H])c43)c([2H])c([2H])c1n2-c1ccc(-c2nc(-c3ccccc3)nc(-c3ccccc3)n2)c(-c2ccccc2)c1 ; [2H]c1c([2H])c([2H])c2c(c1[2H])c1c([2H])c([2H])c([2H])c(-n3c4ccccc4c4ccccc43)c1n2-c1ccc(-c2nc(-c3ccccc3)nc(-c3ccccc3)n2)c(-c2ccccc2)c1]

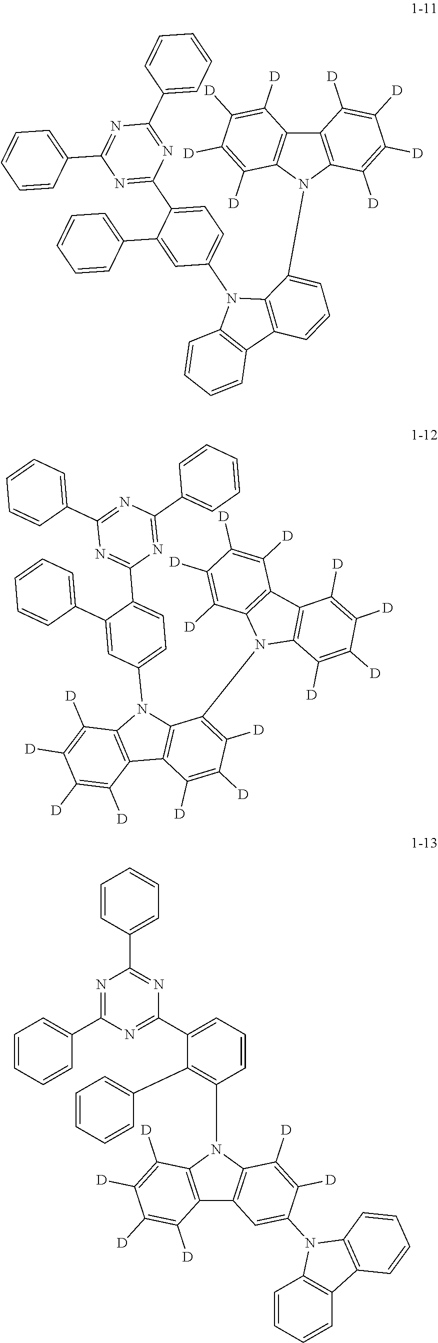 [SMILES: [2H]c1c([2H])c([2H])c2c(c1[2H])c1c([2H])c([2H])c([2H])c([2H])c1n2-c1cccc2c3ccccc3n(-c3ccc(-c4nc(-c5ccccc5)nc(-c5ccccc5)n4)c(-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2nc(-c3ccccc3)nc(-c3ccccc3)n2)c(-c2ccccc2)c1 ; [2H]c1c([2H])c([2H])c2c(c1[2H])c1cc(-n3c4ccccc4c4ccccc43)c([2H])c([2H])c1n2-c1cccc(-c2nc(-c3ccccc3)nc(-c3ccccc3)n2)c1-c1ccccc1]

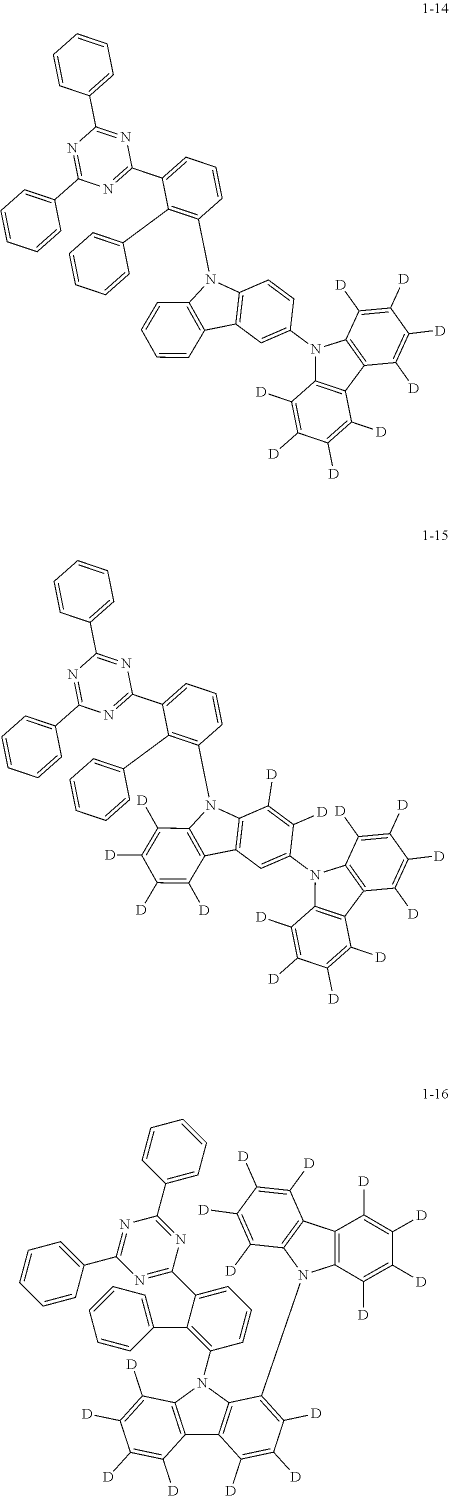 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2nc(-c3ccccc3)nc(-c3ccccc3)n2)c1-c1ccccc1 ; [2H]c1c([2H])c([2H])c2c(c1[2H])c1cc(-n3c4c([2H])c([2H])c([2H])c([2H])c4c4c([2H])c([2H])c([2H])c([2H])c43)c([2H])c([2H])c1n2-c1cccc(-c2nc(-c3ccccc3)nc(-c3ccccc3)n2)c1-c1ccccc1 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2nc(-c3ccccc3)nc(-c3ccccc3)n2)c1-c1ccccc1]

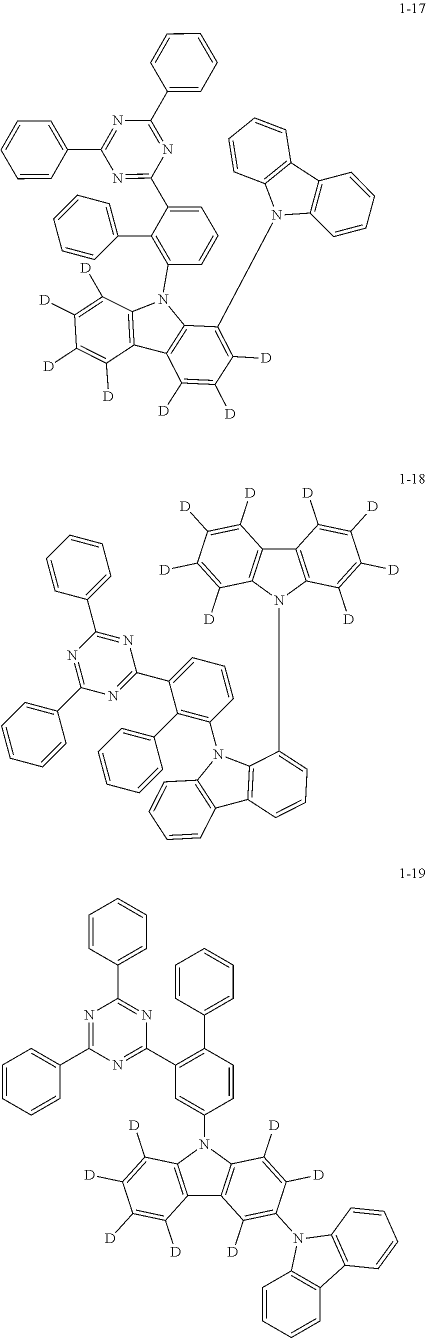 [SMILES: [2H]c1c([2H])c([2H])c2c(c1[2H])c1c([2H])c([2H])c([2H])c(-n3c4ccccc4c4ccccc43)c1n2-c1cccc(-c2nc(-c3ccccc3)nc(-c3ccccc3)n2)c1-c1ccccc1 ; [2H]c1c([2H])c([2H])c2c(c1[2H])c1c([2H])c([2H])c([2H])c([2H])c1n2-c1cccc2c3ccccc3n(-c3cccc(-c4nc(-c5ccccc5)nc(-c5ccccc5)n4)c3-c3ccccc3)c12 ; [2H]c1c([2H])c([2H])c2c(c1[2H])c1c([2H])c(-n3c4ccccc4c4ccccc43)c([2H])c([2H])c1n2-c1ccc(-c2ccccc2)c(-c2nc(-c3ccccc3)nc(-c3ccccc3)n2)c1]

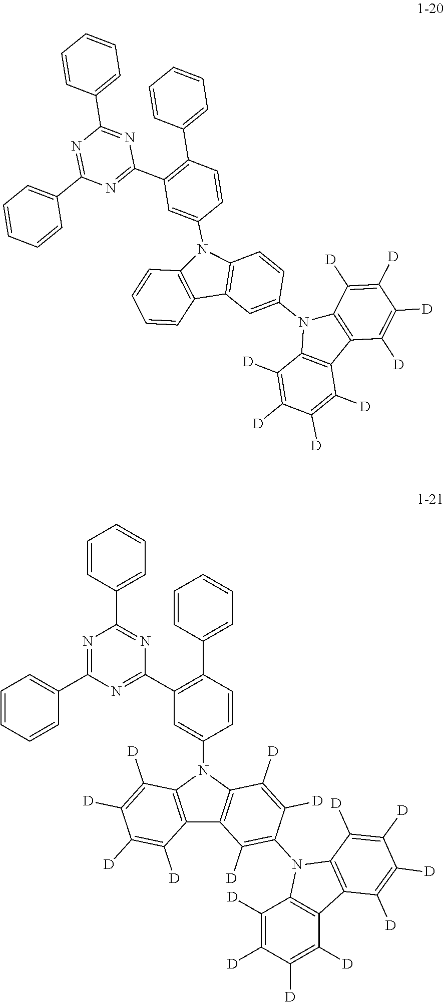 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2ccccc2)c(-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)c(-c2nc(-c3ccccc3)nc(-c3ccccc3)n2)c1]

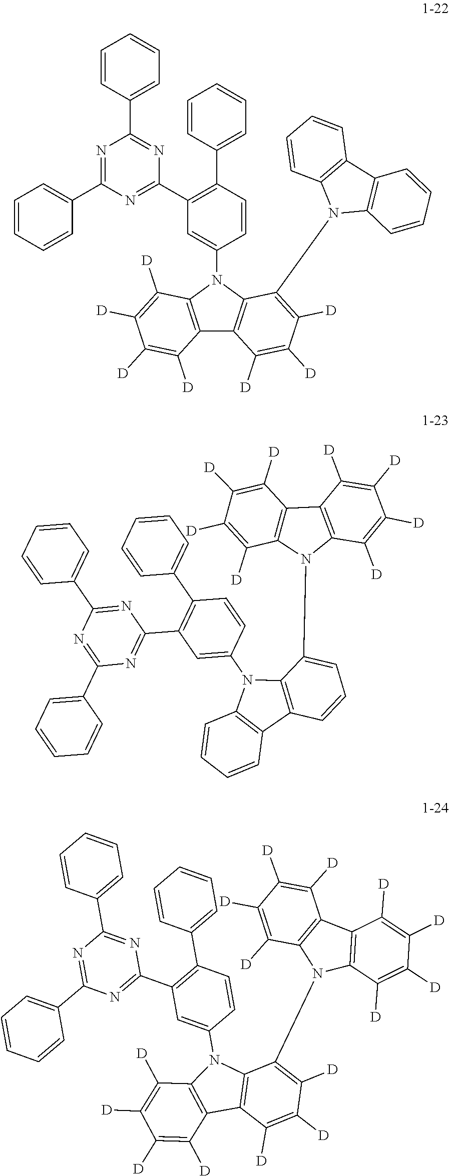 [SMILES: [2H]c1c([2H])c([2H])c2c(c1[2H])c1c([2H])c([2H])c([2H])c(-n3c4ccccc4c4ccccc43)c1n2-c1ccc(-c2ccccc2)c(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3ccc(-c4ccccc4)c(-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)c(-c2nc(-c3ccccc3)nc(-c3ccccc3)n2)c1]

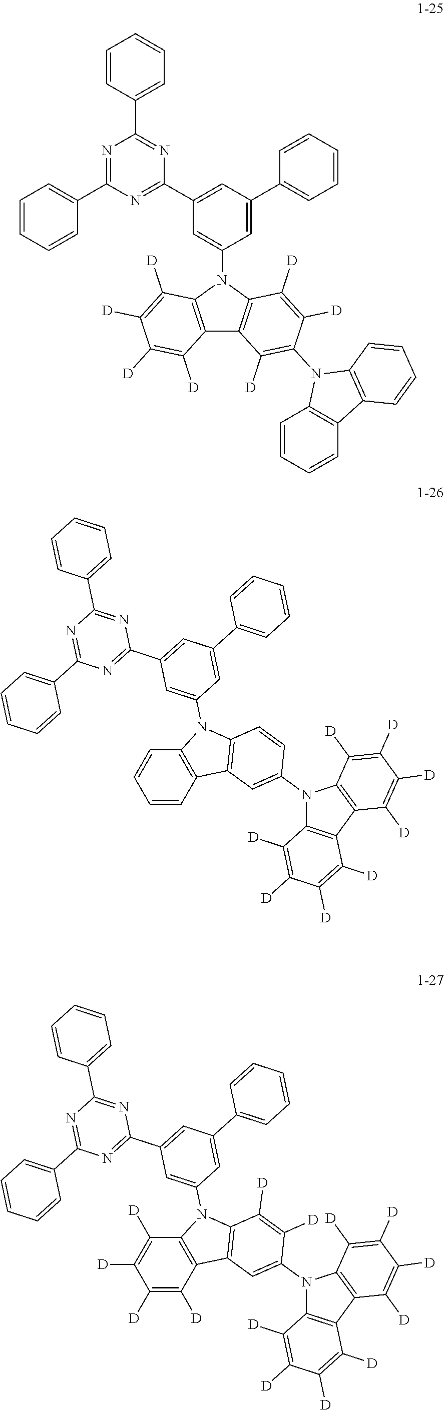 [SMILES: [2H]c1c([2H])c([2H])c2c(c1[2H])c1c([2H])c(-n3c4ccccc4c4ccccc43)c([2H])c([2H])c1n2-c1cc(-c2ccccc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2)cc(-c2nc(-c3ccccc3)nc(-c3ccccc3)n2)c1 ; [2H]c1c([2H])c([2H])c2c(c1[2H])c1cc(-n3c4c([2H])c([2H])c([2H])c([2H])c4c4c([2H])c([2H])c([2H])c([2H])c43)c([2H])c([2H])c1n2-c1cc(-c2ccccc2)cc(-c2nc(-c3ccccc3)nc(-c3ccccc3)n2)c1]

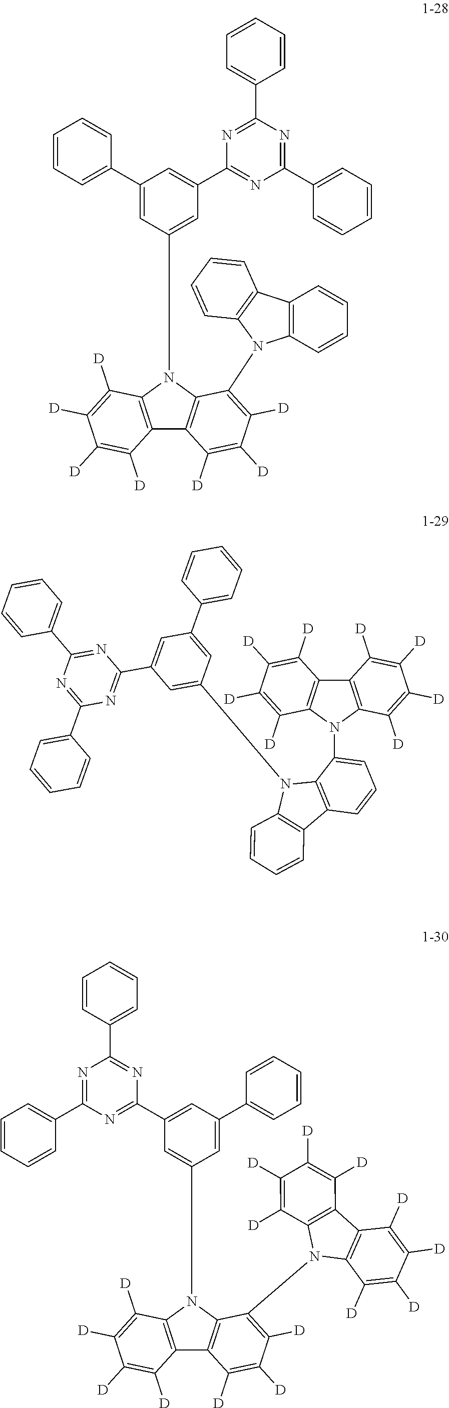 [SMILES: [2H]c1c([2H])c([2H])c2c(c1[2H])c1c([2H])c([2H])c([2H])c(-n3c4ccccc4c4ccccc43)c1n2-c1cc(-c2ccccc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3cc(-c4ccccc4)cc(-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2)cc(-c2nc(-c3ccccc3)nc(-c3ccccc3)n2)c1]

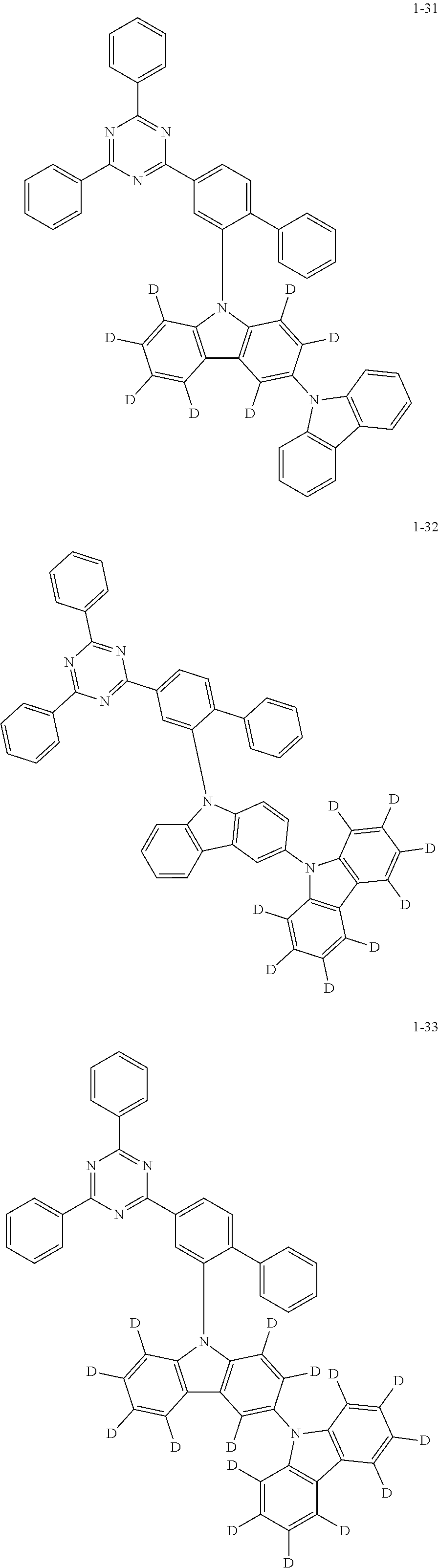 [SMILES: [2H]c1c([2H])c([2H])c2c(c1[2H])c1c([2H])c(-n3c4ccccc4c4ccccc43)c([2H])c([2H])c1n2-c1cc(-c2nc(-c3ccccc3)nc(-c3ccccc3)n2)ccc1-c1ccccc1 ; [2H]c1c([2H])c([2H])c2c(c1[2H])c1c([2H])c([2H])c([2H])c([2H])c1n2-c1ccc2c(c1)c1ccccc1n2-c1cc(-c2nc(-c3ccccc3)nc(-c3ccccc3)n2)ccc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2nc(-c3ccccc3)nc(-c3ccccc3)n2)ccc1-c1ccccc1]

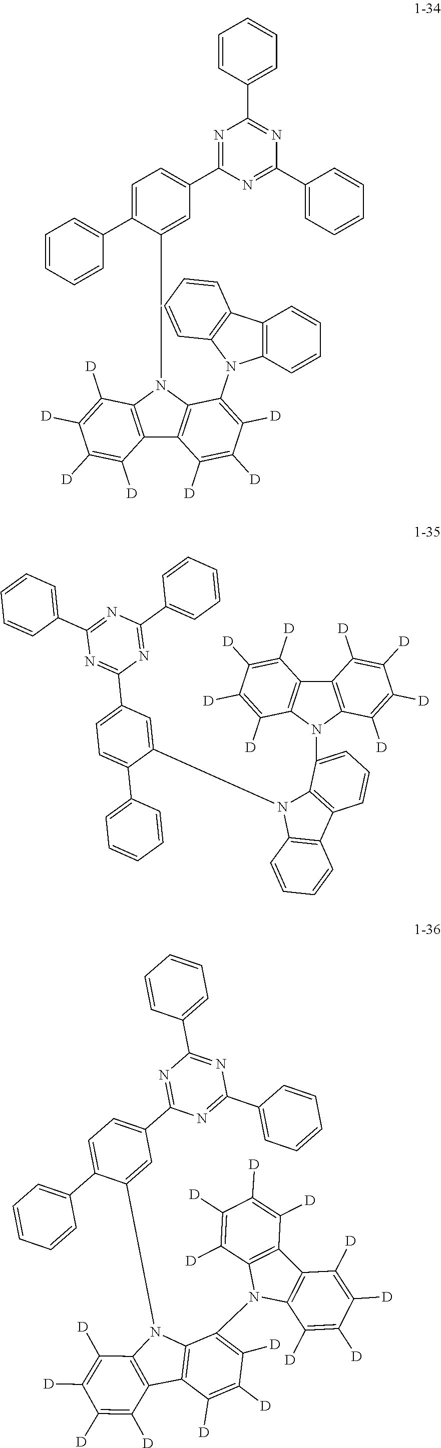 [SMILES: [2H]c1c([2H])c([2H])c2c(c1[2H])c1c([2H])c([2H])c([2H])c(-n3c4ccccc4c4ccccc43)c1n2-c1cc(-c2nc(-c3ccccc3)nc(-c3ccccc3)n2)ccc1-c1ccccc1 ; [2H]c1c([2H])c([2H])c2c(c1[2H])c1c([2H])c([2H])c([2H])c([2H])c1n2-c1cccc2c3ccccc3n(-c3cc(-c4nc(-c5ccccc5)nc(-c5ccccc5)n4)ccc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2nc(-c3ccccc3)nc(-c3ccccc3)n2)ccc1-c1ccccc1]

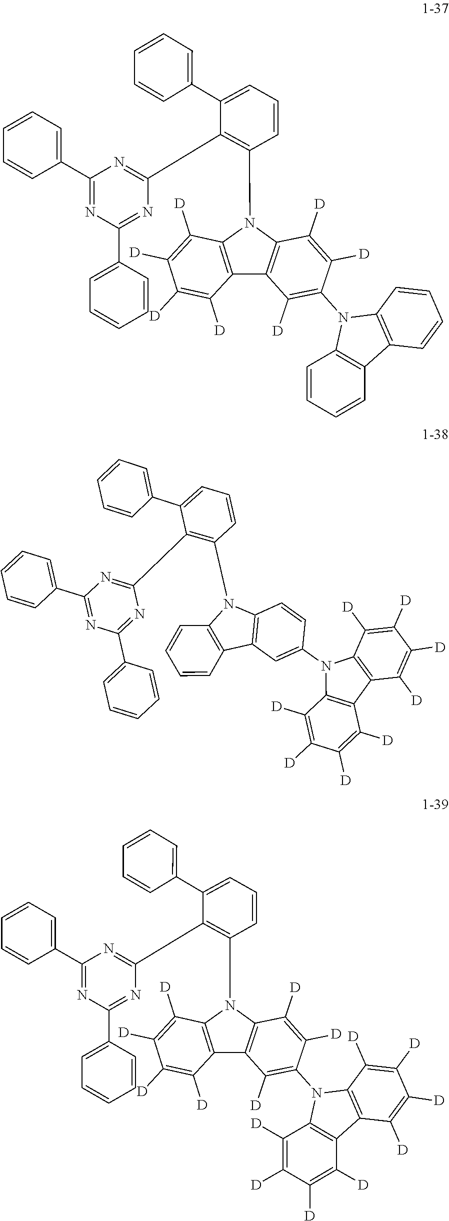 [SMILES: [2H]c1c([2H])c([2H])c2c(c1[2H])c1c([2H])c(-n3c4ccccc4c4ccccc43)c([2H])c([2H])c1n2-c1cccc(-c2ccccc2)c1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1cccc(-c2ccccc2)c1-c1nc(-c2ccccc2)nc(-c2ccccc2)n1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccccc2)c1-c1nc(-c2ccccc2)nc(-c2ccccc2)n1]

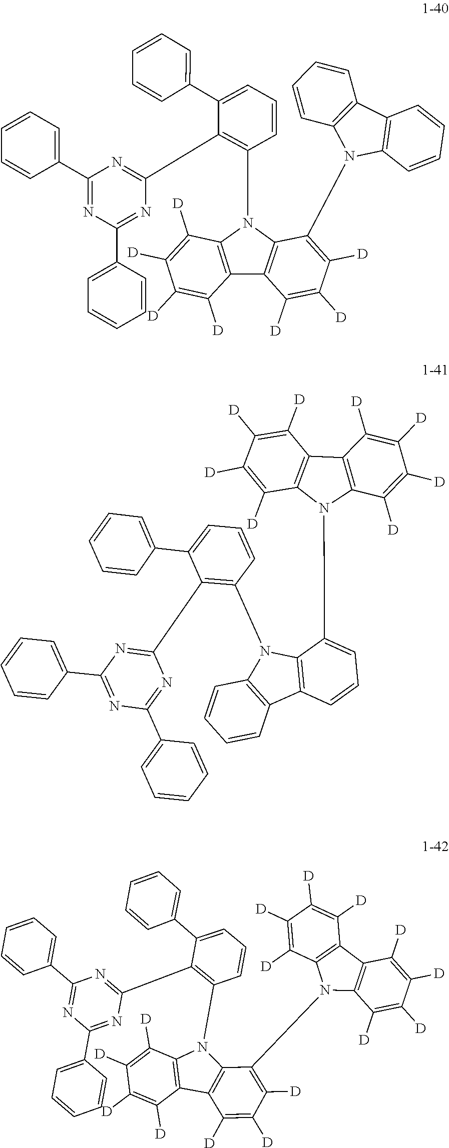 [SMILES: [2H]c1c([2H])c([2H])c2c(c1[2H])c1c([2H])c([2H])c([2H])c(-n3c4ccccc4c4ccccc43)c1n2-c1cccc(-c2ccccc2)c1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3cccc(-c4ccccc4)c3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccccc2)c1-c1nc(-c2ccccc2)nc(-c2ccccc2)n1]

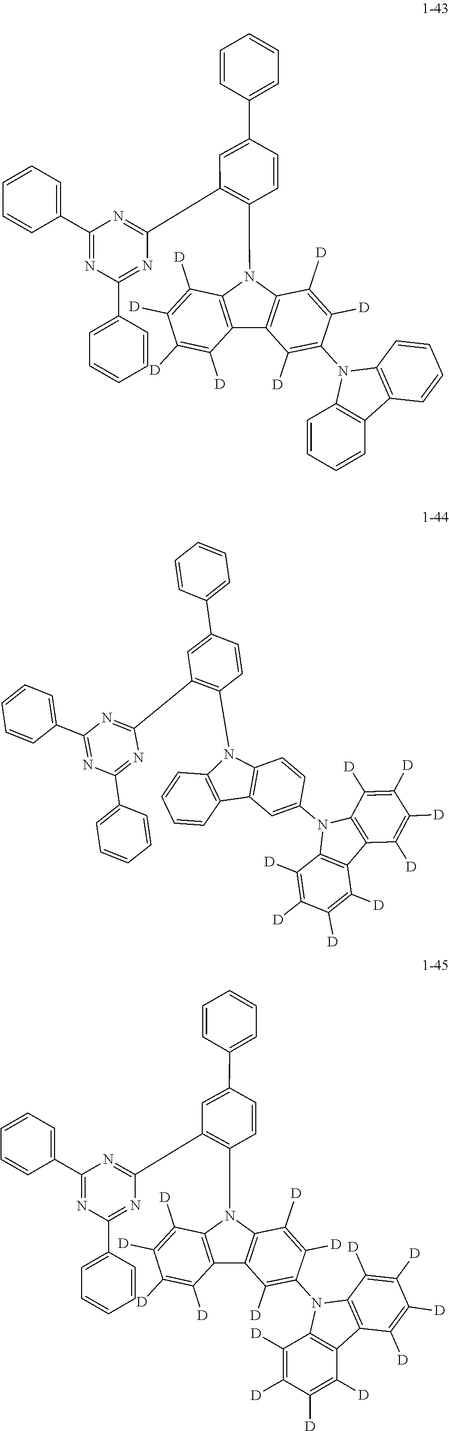 [SMILES: [2H]c1c([2H])c([2H])c2c(c1[2H])c1c([2H])c(-n3c4ccccc4c4ccccc43)c([2H])c([2H])c1n2-c1ccc(-c2ccccc2)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1ccc(-c2ccccc2)cc1-c1nc(-c2ccccc2)nc(-c2ccccc2)n1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)cc1-c1nc(-c2ccccc2)nc(-c2ccccc2)n1]

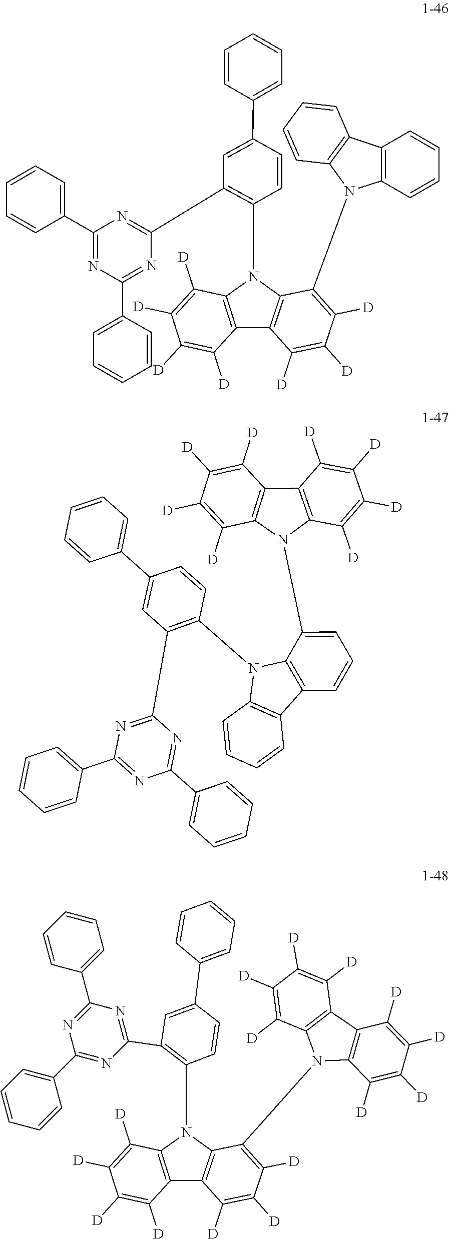 [SMILES: [2H]c1c([2H])c([2H])c2c(c1[2H])c1c([2H])c([2H])c([2H])c(-n3c4ccccc4c4ccccc43)c1n2-c1ccc(-c2ccccc2)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3ccc(-c4ccccc4)cc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)cc1-c1nc(-c2ccccc2)nc(-c2ccccc2)n1]

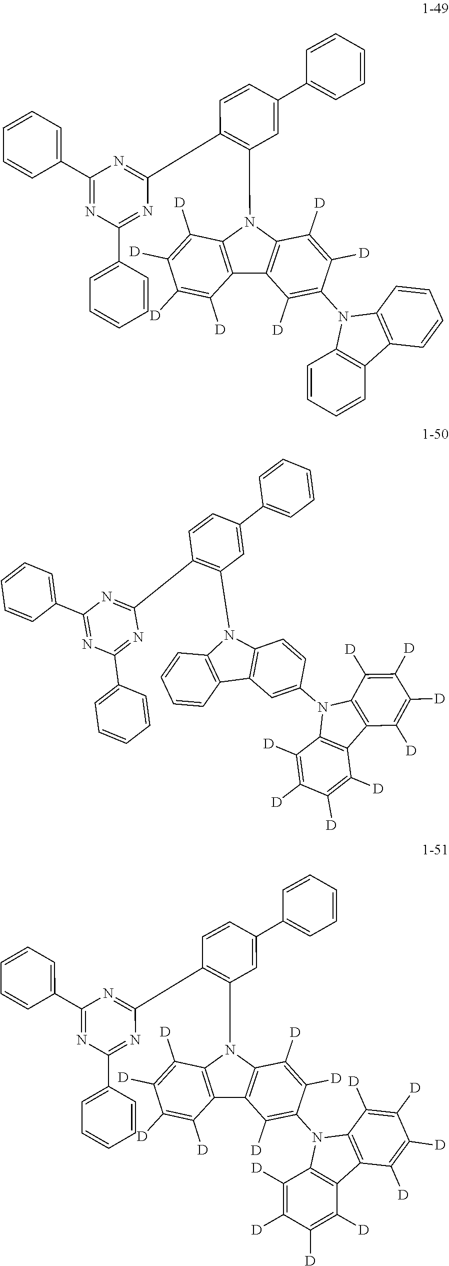 [SMILES: [2H]c1c([2H])c([2H])c2c(c1[2H])c1c([2H])c(-n3c4ccccc4c4ccccc43)c([2H])c([2H])c1n2-c1cc(-c2ccccc2)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2)ccc1-c1nc(-c2ccccc2)nc(-c2ccccc2)n1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)ccc1-c1nc(-c2ccccc2)nc(-c2ccccc2)n1]

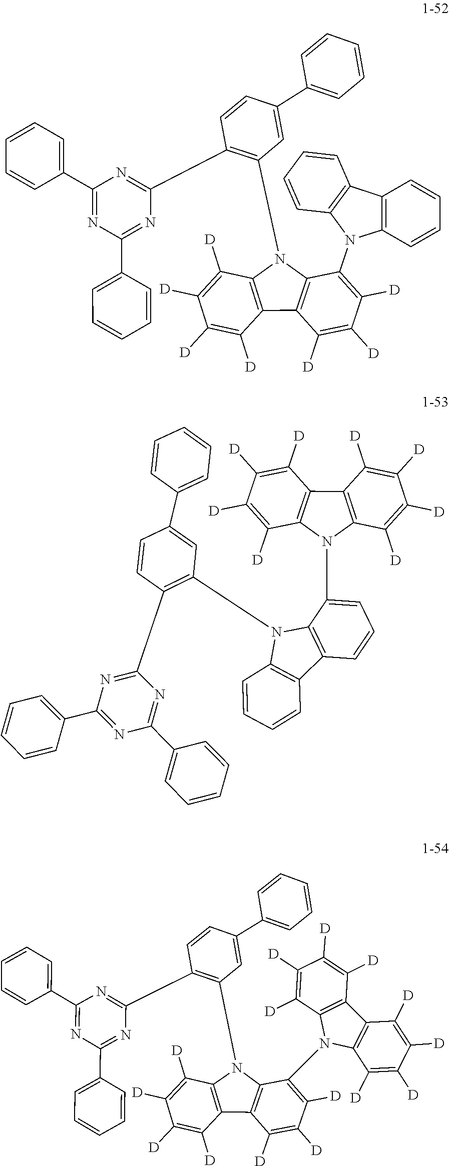 [SMILES: [2H]c1c([2H])c([2H])c2c(c1[2H])c1c([2H])c([2H])c([2H])c(-n3c4ccccc4c4ccccc43)c1n2-c1cc(-c2ccccc2)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3cc(-c4ccccc4)ccc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2)ccc1-c1nc(-c2ccccc2)nc(-c2ccccc2)n1]

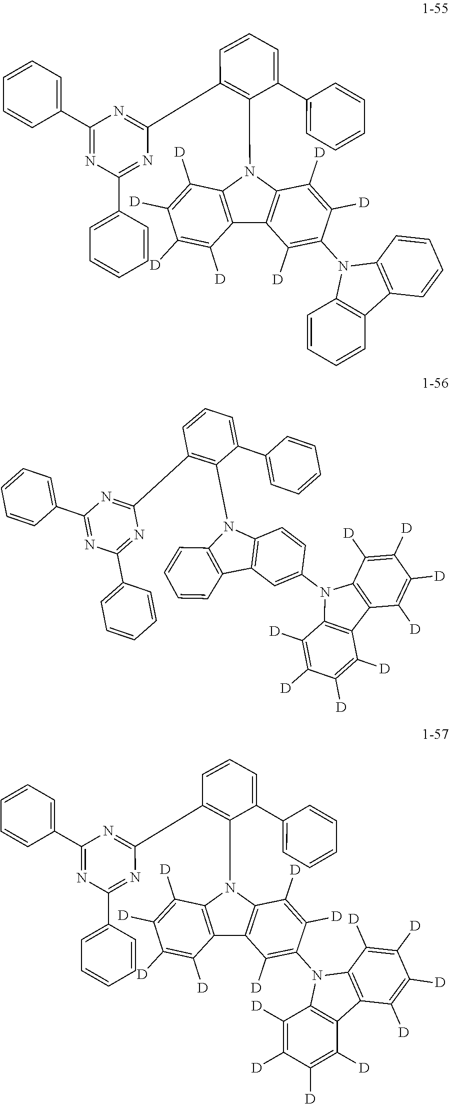 [SMILES: [2H]c1c([2H])c([2H])c2c(c1[2H])c1c([2H])c(-n3c4ccccc4c4ccccc43)c([2H])c([2H])c1n2-c1c(-c2ccccc2)cccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1c(-c2ccccc2)cccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1c(-c2ccccc2)cccc1-c1nc(-c2ccccc2)nc(-c2ccccc2)n1]

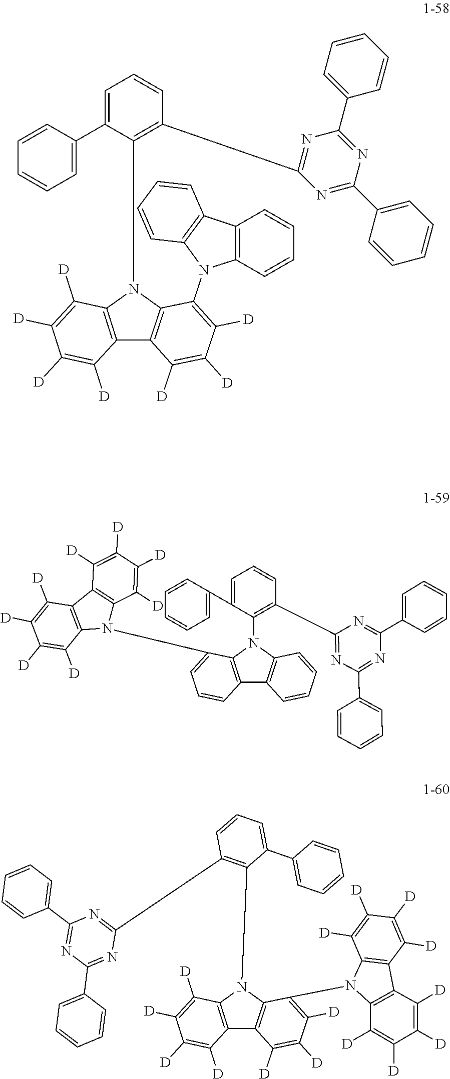 [SMILES: [2H]c1c([2H])c([2H])c2c(c1[2H])c1c([2H])c([2H])c([2H])c(-n3c4ccccc4c4ccccc43)c1n2-c1c(-c2ccccc2)cccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3c(-c4ccccc4)cccc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1c(-c2ccccc2)cccc1-c1nc(-c2ccccc2)nc(-c2ccccc2)n1]

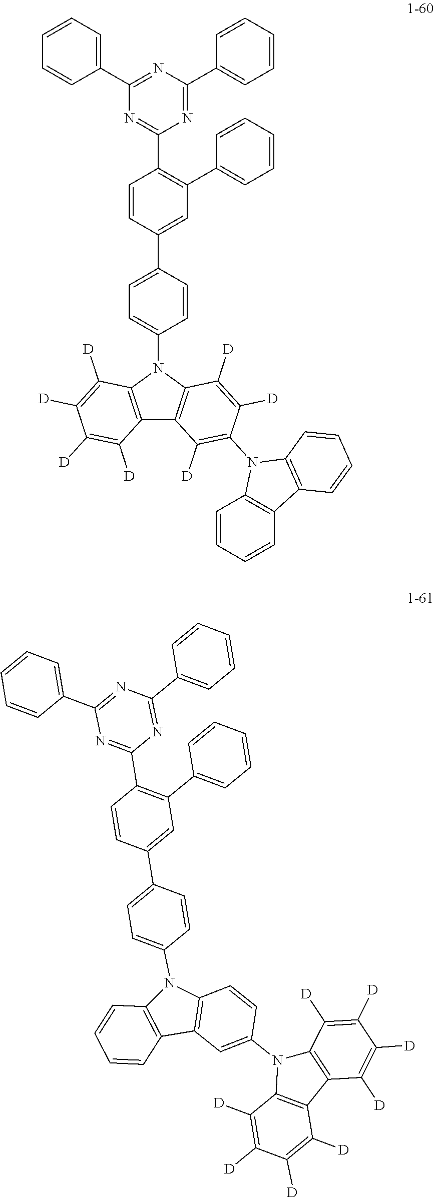 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3nc(-c4ccccc4)nc(-c4ccccc4)n3)c(-c3ccccc3)c2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3nc(-c4ccccc4)nc(-c4ccccc4)n3)c(-c3ccccc3)c2)cc1]

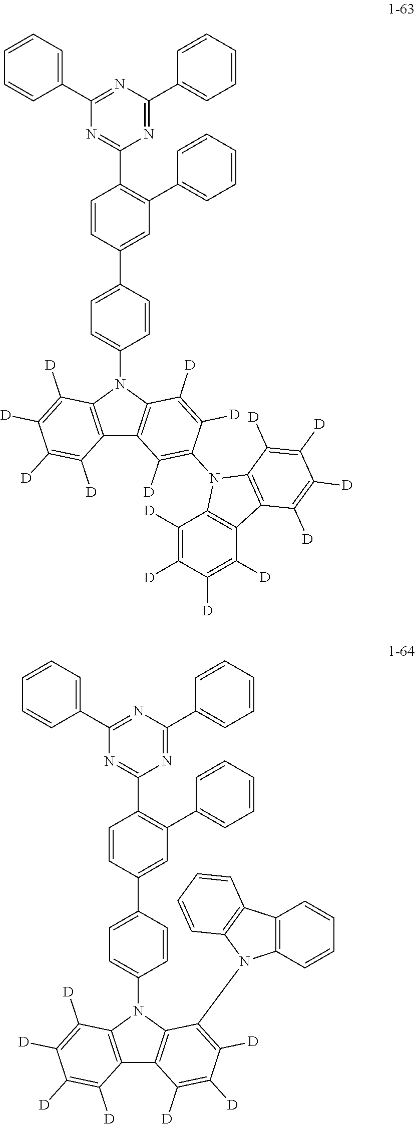 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)c(-c3ccccc3)c2)cc1 ; [2H]c1c([2H])c([2H])c2c(c1[2H])c1c([2H])c([2H])c([2H])c(-n3c4ccccc4c4ccccc43)c1n2-c1ccc(-c2ccc(-c3nc(-c4ccccc4)nc(-c4ccccc4)n3)c(-c3ccccc3)c2)cc1]

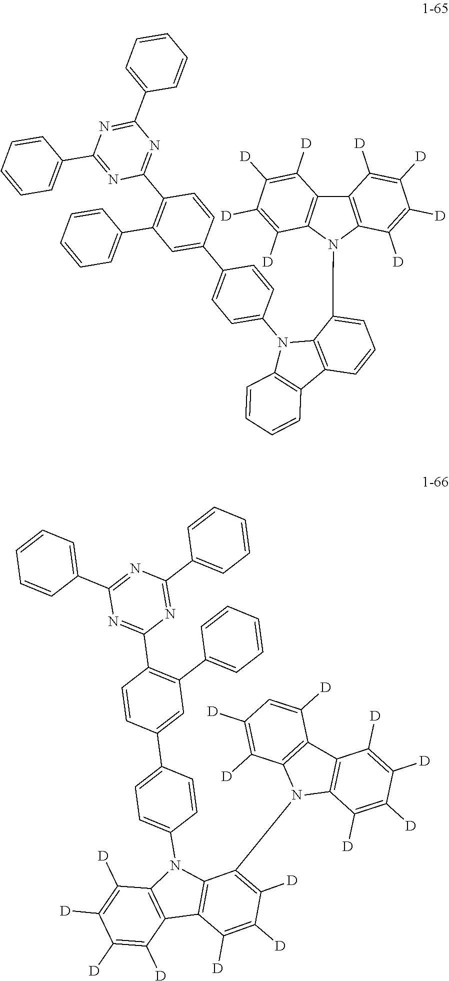 [SMILES: [2H]c1c([2H])c([2H])c2c(c1[2H])c1c([2H])c([2H])c([2H])c([2H])c1n2-c1cccc2c3ccccc3n(-c3ccc(-c4ccc(-c5nc(-c6ccccc6)nc(-c6ccccc6)n5)c(-c5ccccc5)c4)cc3)c12 ; [2H]c1cc([2H])c2c3c([2H])c([2H])c([2H])c([2H])c3n(-c3c([2H])c([2H])c([2H])c4c5c([2H])c([2H])c([2H])c([2H])c5n(-c5ccc(-c6ccc(-c7nc(-c8ccccc8)nc(-c8ccccc8)n7)c(-c7ccccc7)c6)cc5)c34)c2c1[2H]]

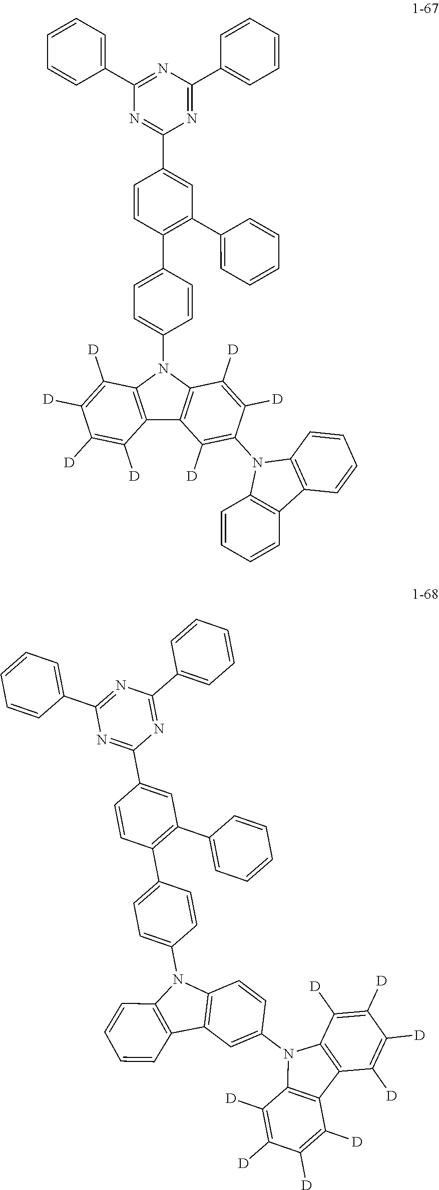 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3nc(-c4ccccc4)nc(-c4ccccc4)n3)cc2-c2ccccc2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3nc(-c4ccccc4)nc(-c4ccccc4)n3)cc2-c2ccccc2)cc1]

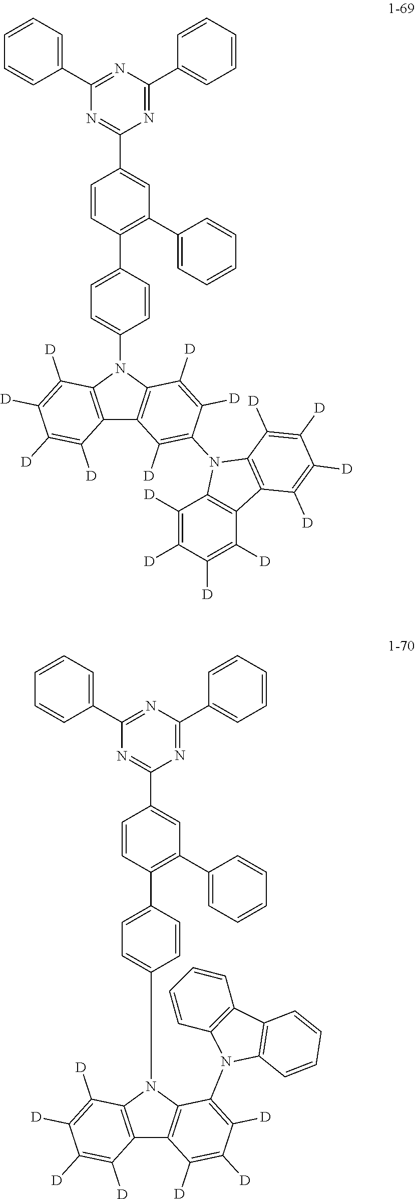 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2-c2ccccc2)cc1 ; [2H]c1c([2H])c([2H])c2c(c1[2H])c1c([2H])c([2H])c([2H])c(-n3c4ccccc4c4ccccc43)c1n2-c1ccc(-c2ccc(-c3nc(-c4ccccc4)nc(-c4ccccc4)n3)cc2-c2ccccc2)cc1]

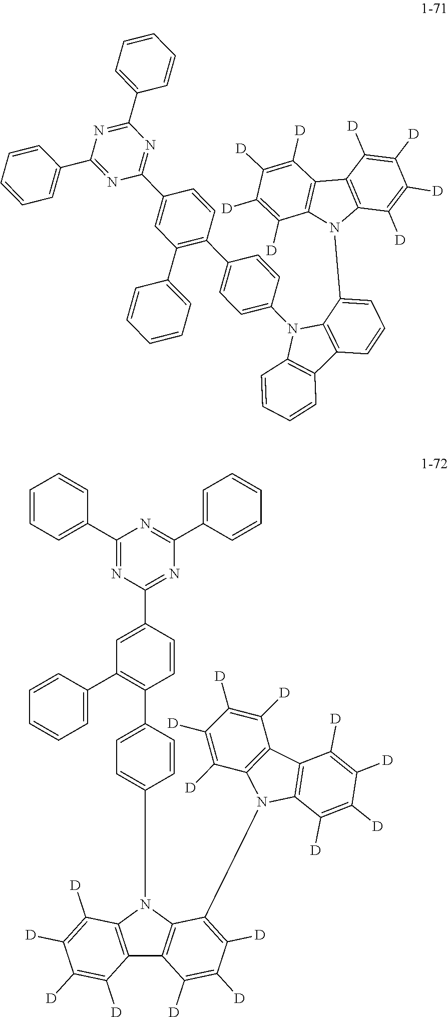 [SMILES: [2H]c1c([2H])c([2H])c2c(c1[2H])c1c([2H])c([2H])c([2H])c([2H])c1n2-c1cccc2c3ccccc3n(-c3ccc(-c4ccc(-c5nc(-c6ccccc6)nc(-c6ccccc6)n5)cc4-c4ccccc4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)cc2-c2ccccc2)cc1]

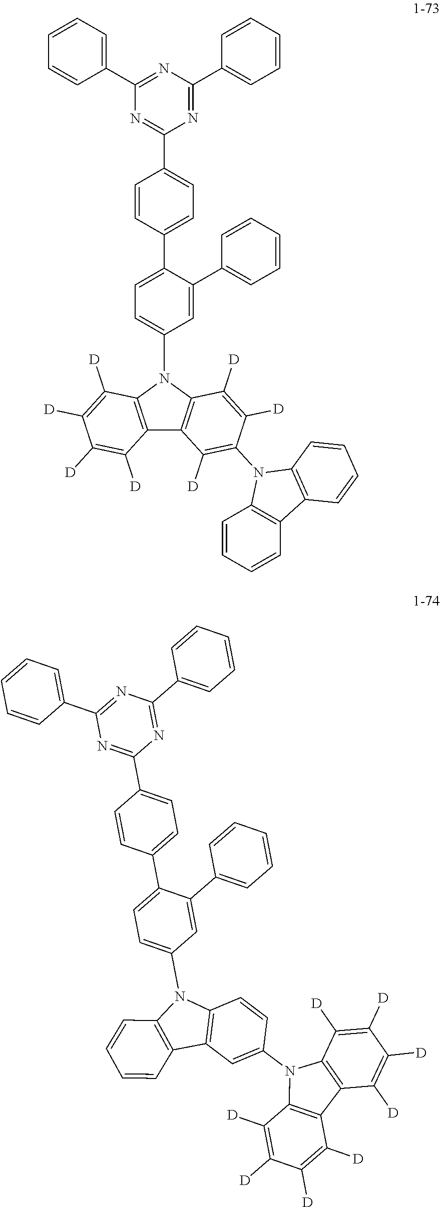 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3nc(-c4ccccc4)nc(-c4ccccc4)n3)cc2)c(-c2ccccc2)c1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3nc(-c4ccccc4)nc(-c4ccccc4)n3)cc2)c(-c2ccccc2)c1]

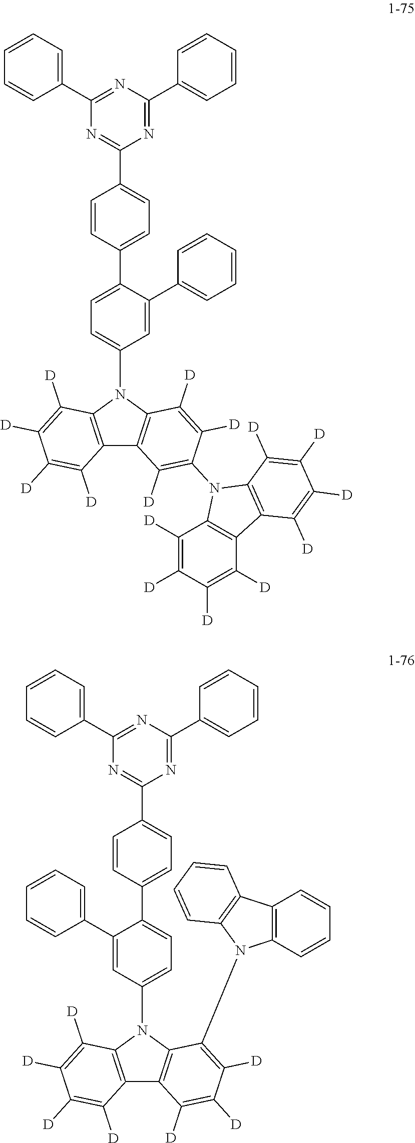 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)c(-c2ccccc2)c1 ; [2H]c1c([2H])c([2H])c2c(c1[2H])c1c([2H])c([2H])c([2H])c(-n3c4ccccc4c4ccccc43)c1n2-c1ccc(-c2ccc(-c3nc(-c4ccccc4)nc(-c4ccccc4)n3)cc2)c(-c2ccccc2)c1]

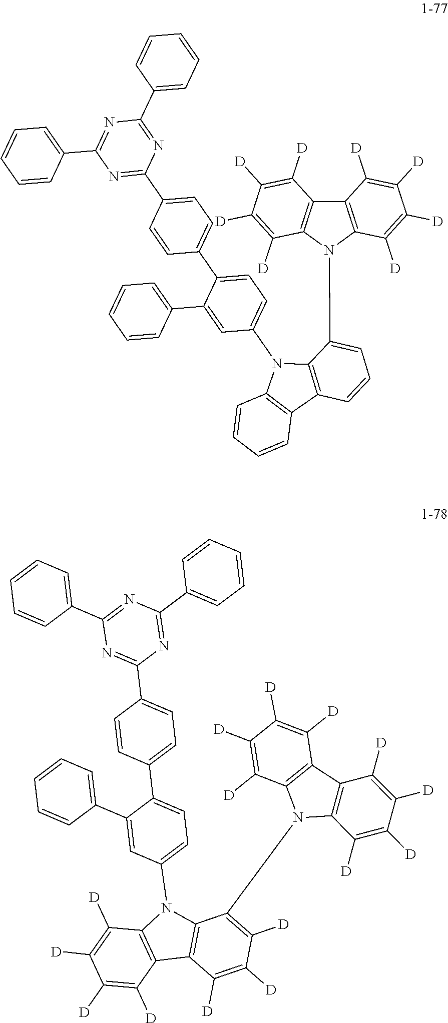 [SMILES: [2H]c1c([2H])c([2H])c2c(c1[2H])c1c([2H])c([2H])c([2H])c([2H])c1n2-c1cccc2c3ccccc3n(-c3ccc(-c4ccc(-c5nc(-c6ccccc6)nc(-c6ccccc6)n5)cc4)c(-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)cc2)c(-c2ccccc2)c1]

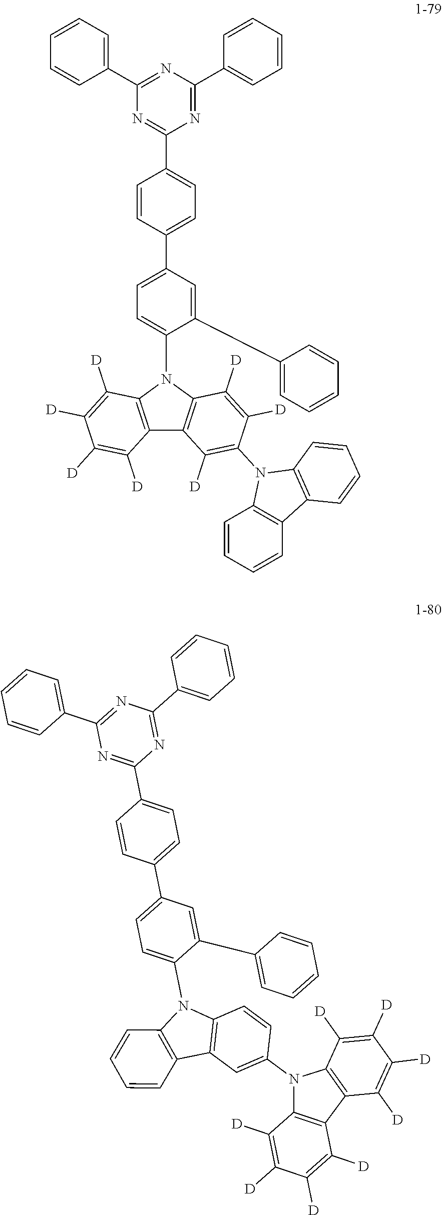 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3nc(-c4ccccc4)nc(-c4ccccc4)n3)cc2)cc1-c1ccccc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3nc(-c4ccccc4)nc(-c4ccccc4)n3)cc2)cc1-c1ccccc1]

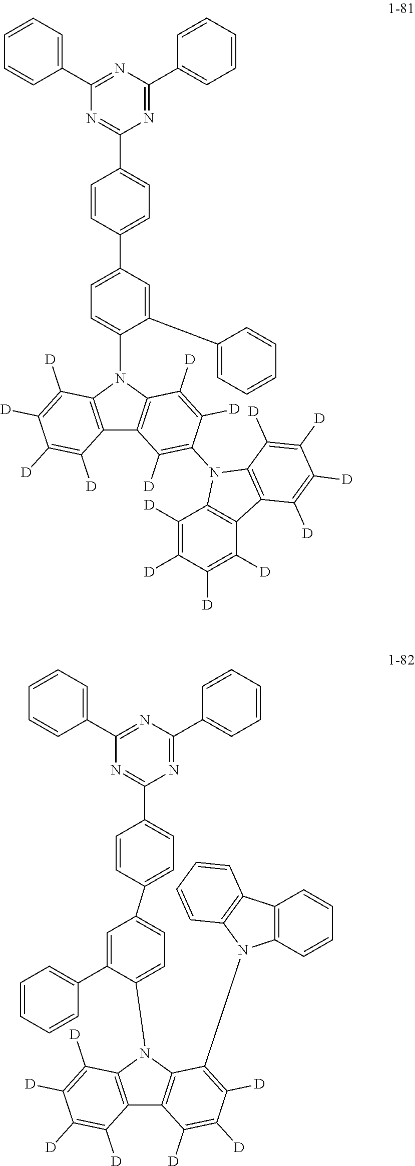 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)cc1-c1ccccc1 ; [2H]c1c([2H])c([2H])c2c(c1[2H])c1c([2H])c([2H])c([2H])c(-n3c4ccccc4c4ccccc43)c1n2-c1ccc(-c2ccc(-c3nc(-c4ccccc4)nc(-c4ccccc4)n3)cc2)cc1-c1ccccc1]

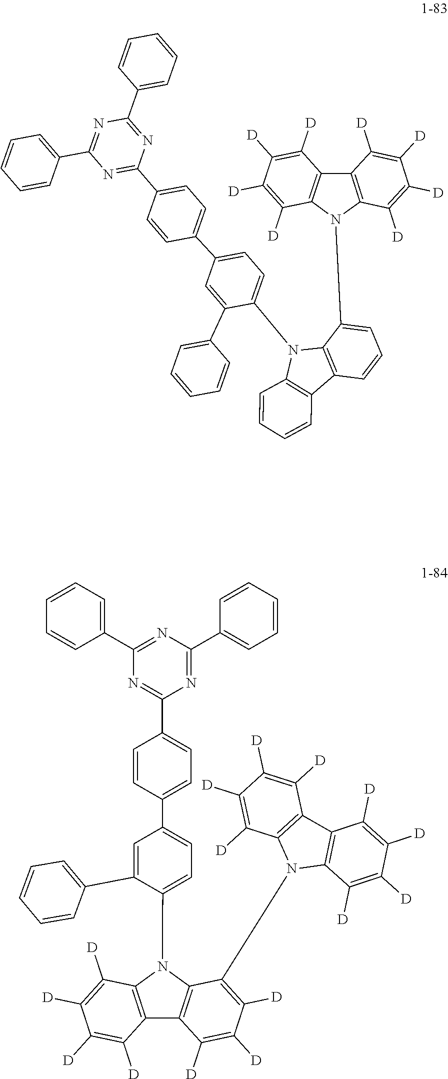 [SMILES: [2H]c1c([2H])c([2H])c2c(c1[2H])c1c([2H])c([2H])c([2H])c([2H])c1n2-c1cccc2c3ccccc3n(-c3ccc(-c4ccc(-c5nc(-c6ccccc6)nc(-c6ccccc6)n5)cc4)cc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)cc2)cc1-c1ccccc1]

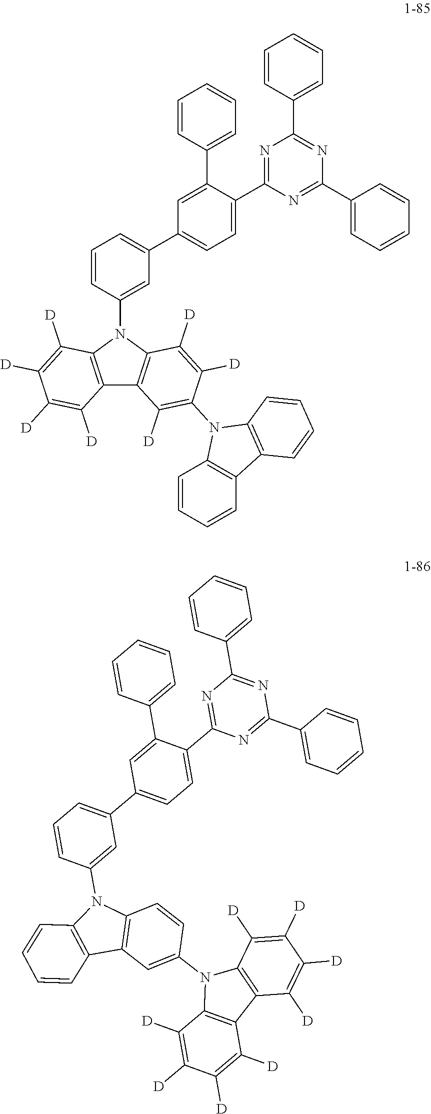 [SMILES: [2H]c1c([2H])c([2H])c2c(c1[2H])c1c([2H])c(-n3c4ccccc4c4ccccc43)c([2H])c([2H])c1n2-c1cccc(-c2ccc(-c3nc(-c4ccccc4)nc(-c4ccccc4)n3)c(-c3ccccc3)c2)c1 ; [2H]c1c([2H])c([2H])c2c(c1[2H])c1c([2H])c([2H])c([2H])c([2H])c1n2-c1ccc2c(c1)c1ccccc1n2-c1cccc(-c2ccc(-c3nc(-c4ccccc4)nc(-c4ccccc4)n3)c(-c3ccccc3)c2)c1]

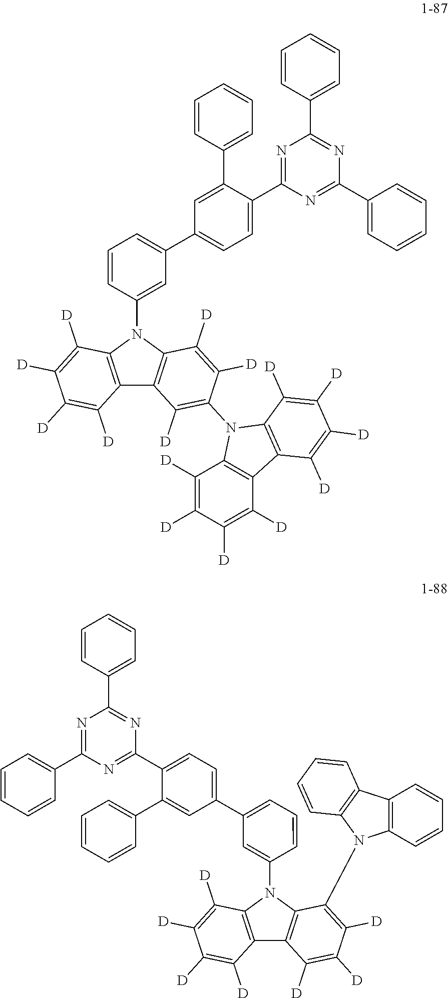 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccc(-c3nc(-c4ccccc4)nc(-c4ccccc4)n3)c(-c3ccccc3)c2)c1 ; [2H]c1c([2H])c([2H])c2c(c1[2H])c1c([2H])c([2H])c([2H])c(-n3c4ccccc4c4ccccc43)c1n2-c1cccc(-c2ccc(-c3nc(-c4ccccc4)nc(-c4ccccc4)n3)c(-c3ccccc3)c2)c1]

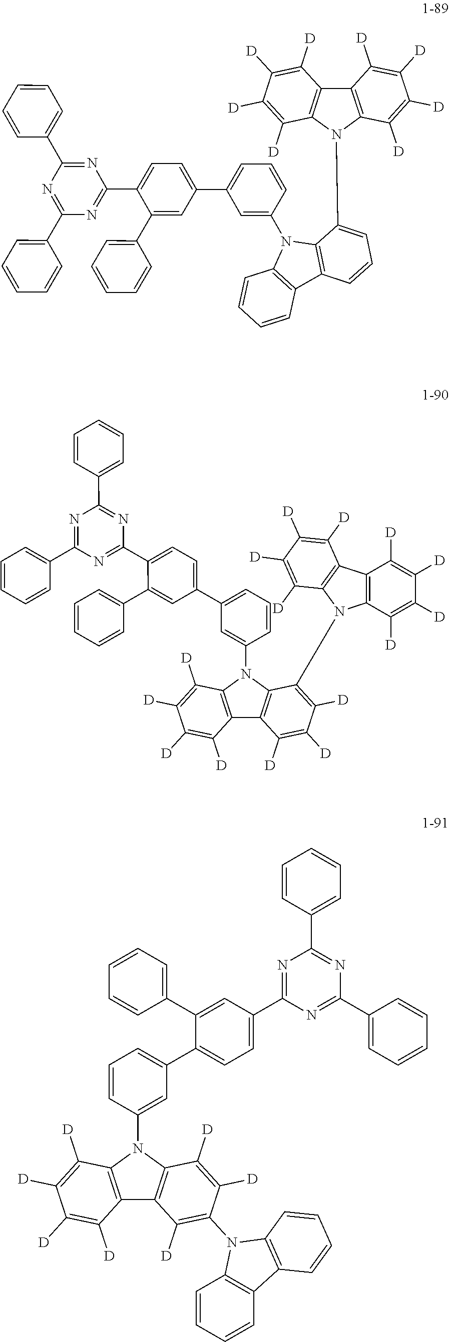 [SMILES: [2H]c1c([2H])c([2H])c2c(c1[2H])c1c([2H])c([2H])c([2H])c([2H])c1n2-c1cccc2c3ccccc3n(-c3cccc(-c4ccc(-c5nc(-c6ccccc6)nc(-c6ccccc6)n5)c(-c5ccccc5)c4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccc(-c3nc(-c4ccccc4)nc(-c4ccccc4)n3)c(-c3ccccc3)c2)c1 ; [2H]c1c([2H])c([2H])c2c(c1[2H])c1c([2H])c(-n3c4ccccc4c4ccccc43)c([2H])c([2H])c1n2-c1cccc(-c2ccc(-c3nc(-c4ccccc4)nc(-c4ccccc4)n3)cc2-c2ccccc2)c1]

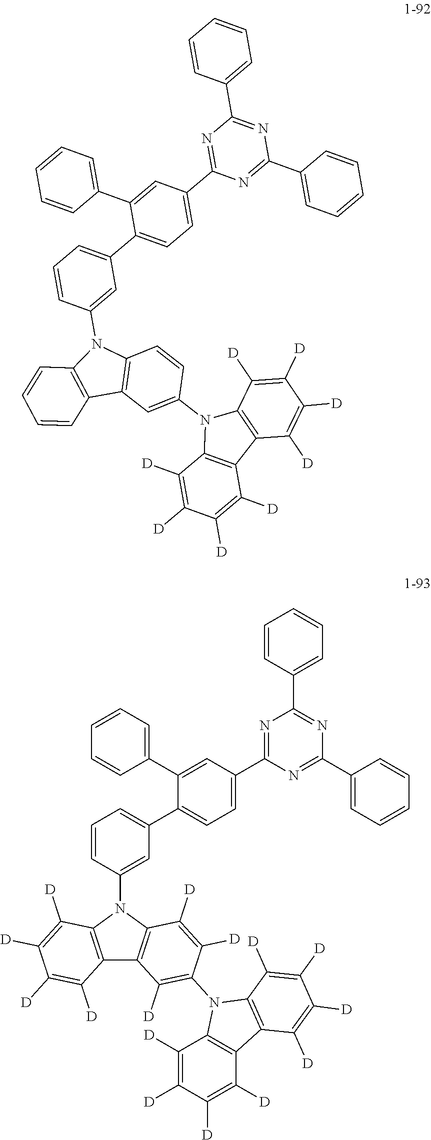 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2ccc(-c3nc(-c4ccccc4)nc(-c4ccccc4)n3)cc2-c2ccccc2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccc(-c3nc(-c4ccccc4)nc(-c4ccccc4)n3)cc2-c2ccccc2)c1]

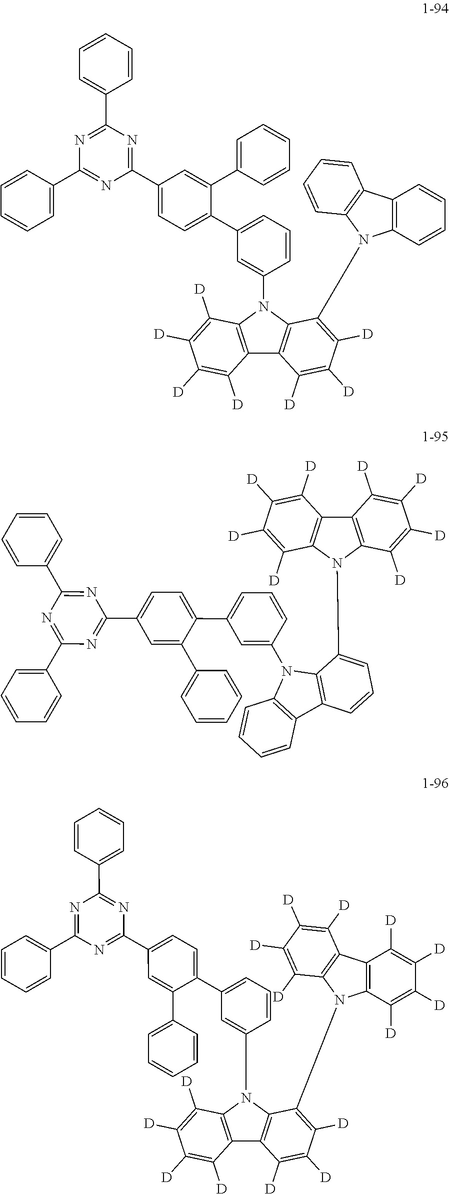 [SMILES: [2H]c1c([2H])c([2H])c2c(c1[2H])c1c([2H])c([2H])c([2H])c(-n3c4ccccc4c4ccccc43)c1n2-c1cccc(-c2ccc(-c3nc(-c4ccccc4)nc(-c4ccccc4)n3)cc2-c2ccccc2)c1 ; [2H]c1c([2H])c([2H])c2c(c1[2H])c1c([2H])c([2H])c([2H])c([2H])c1n2-c1cccc2c3ccccc3n(-c3cccc(-c4ccc(-c5nc(-c6ccccc6)nc(-c6ccccc6)n5)cc4-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccc(-c3nc(-c4ccccc4)nc(-c4ccccc4)n3)cc2-c2ccccc2)c1]

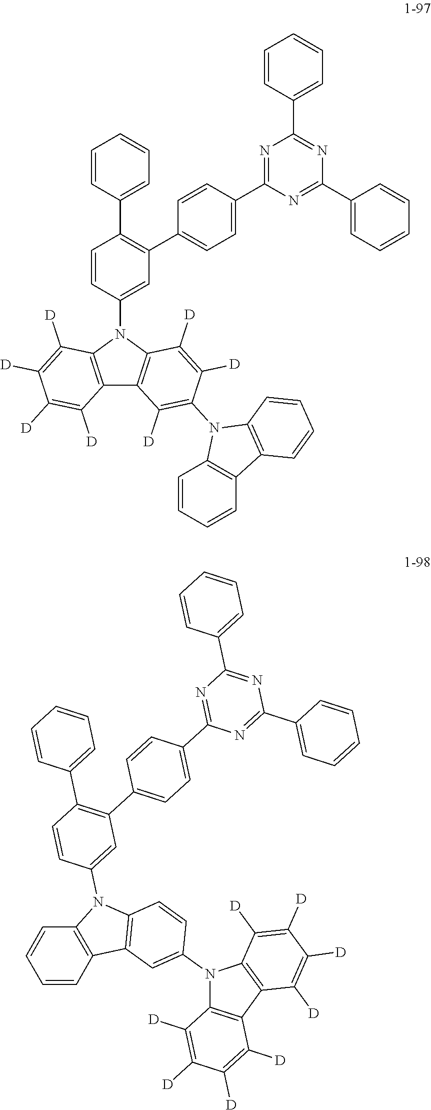

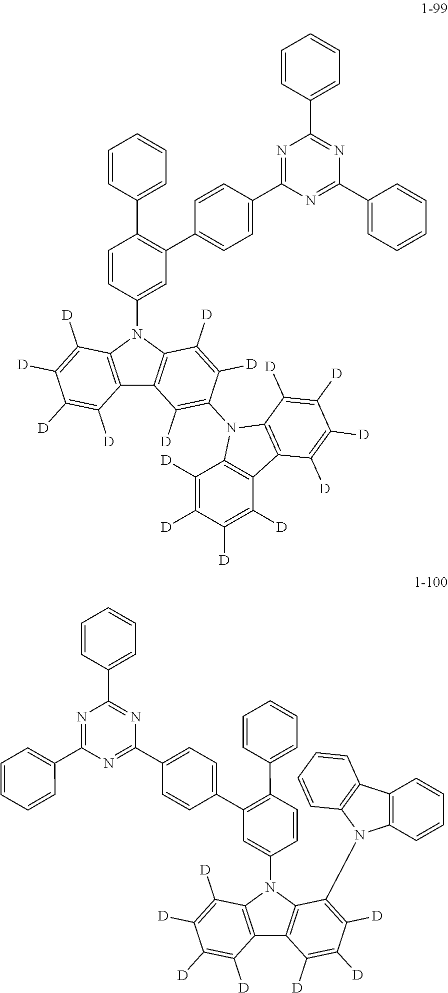 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)c(-c2ccc(-c3nc(-c4ccccc4)nc(-c4ccccc4)n3)cc2)c1 ; [2H]c1c([2H])c([2H])c2c(c1[2H])c1c([2H])c([2H])c([2H])c(-n3c4ccccc4c4ccccc43)c1n2-c1ccc(-c2ccccc2)c(-c2ccc(-c3nc(-c4ccccc4)nc(-c4ccccc4)n3)cc2)c1]

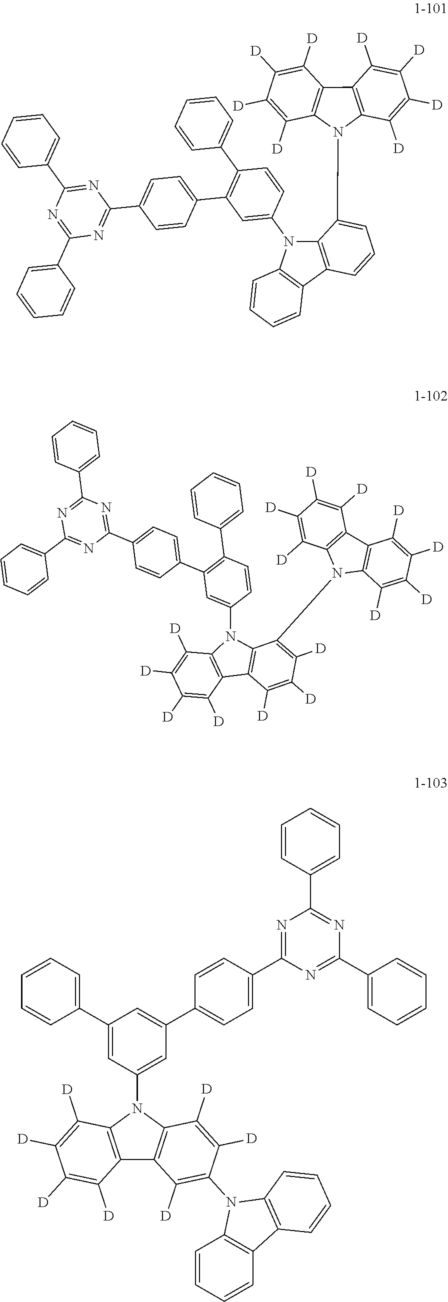 [SMILES: [2H]c1c([2H])c([2H])c2c(c1[2H])c1c([2H])c([2H])c([2H])c([2H])c1n2-c1cccc2c3ccccc3n(-c3ccc(-c4ccccc4)c(-c4ccc(-c5nc(-c6ccccc6)nc(-c6ccccc6)n5)cc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)c(-c2ccc(-c3nc(-c4ccccc4)nc(-c4ccccc4)n3)cc2)c1 ; [2H]c1c([2H])c([2H])c2c(c1[2H])c1c([2H])c(-n3c4ccccc4c4ccccc43)c([2H])c([2H])c1n2-c1cc(-c2ccccc2)cc(-c2ccc(-c3nc(-c4ccccc4)nc(-c4ccccc4)n3)cc2)c1]

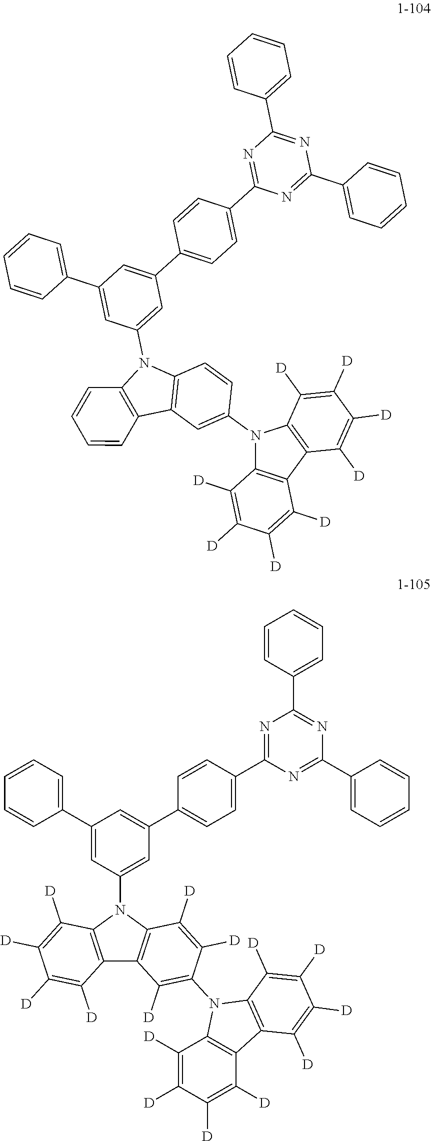 [SMILES: [2H]c1c([2H])c([2H])c2c(c1[2H])c1c([2H])c([2H])c([2H])c([2H])c1n2-c1ccc2c(c1)c1ccccc1n2-c1cc(-c2ccccc2)cc(-c2ccc(-c3nc(-c4ccccc4)nc(-c4ccccc4)n3)cc2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)cc(-c2ccc(-c3nc(-c4ccccc4)nc(-c4ccccc4)n3)cc2)c1]

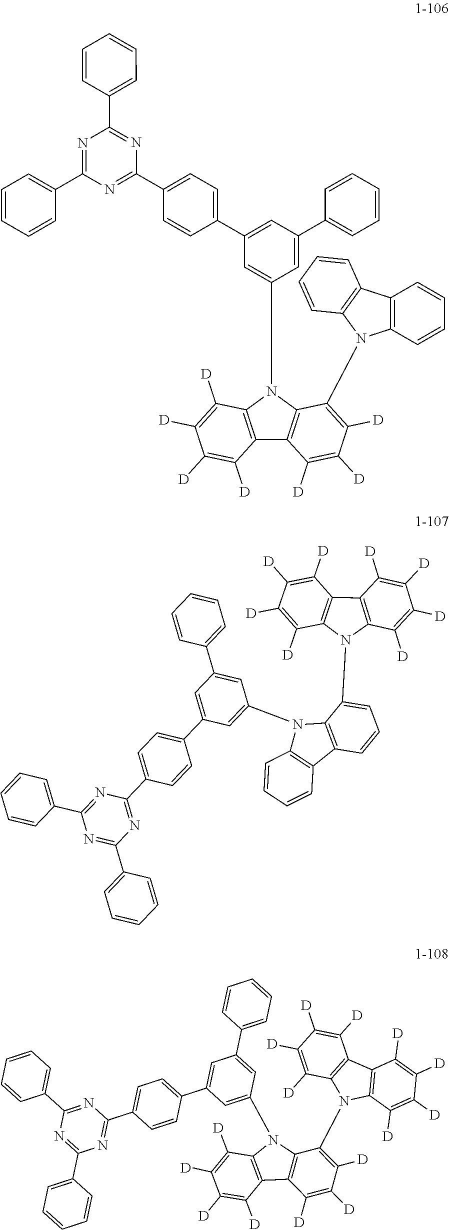 [SMILES: [2H]c1c([2H])c([2H])c2c(c1[2H])c1c([2H])c([2H])c([2H])c(-n3c4ccccc4c4ccccc43)c1n2-c1cc(-c2ccccc2)cc(-c2ccc(-c3nc(-c4ccccc4)nc(-c4ccccc4)n3)cc2)c1 ; [2H]c1c([2H])c([2H])c2c(c1[2H])c1c([2H])c([2H])c([2H])c([2H])c1n2-c1cccc2c3ccccc3n(-c3cc(-c4ccccc4)cc(-c4ccc(-c5nc(-c6ccccc6)nc(-c6ccccc6)n5)cc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2)cc(-c2ccc(-c3nc(-c4ccccc4)nc(-c4ccccc4)n3)cc2)c1]

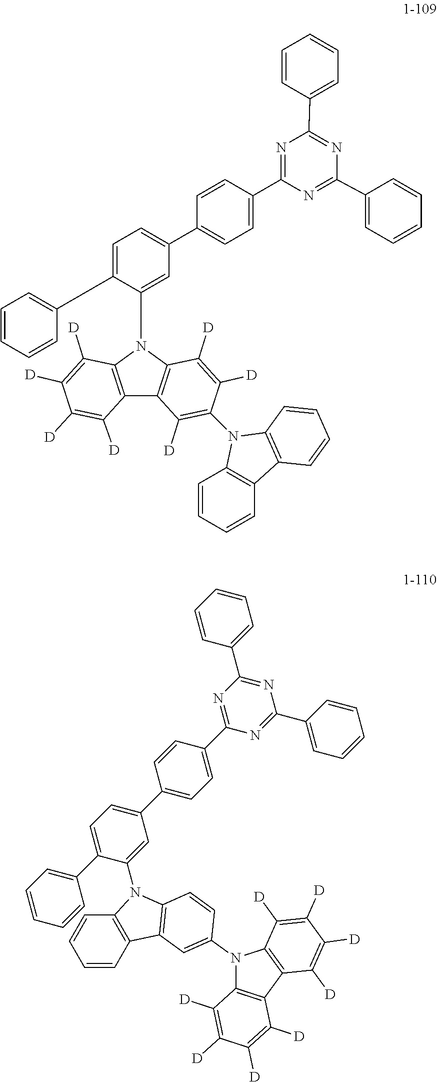 [SMILES: [2H]c1c([2H])c([2H])c2c(c1[2H])c1c([2H])c(-n3c4ccccc4c4ccccc43)c([2H])c([2H])c1n2-c1cc(-c2ccc(-c3nc(-c4ccccc4)nc(-c4ccccc4)n3)cc2)ccc1-c1ccccc1 ; [2H]c1c([2H])c([2H])c2c(c1[2H])c1c([2H])c([2H])c([2H])c([2H])c1n2-c1ccc2c(c1)c1ccccc1n2-c1cc(-c2ccc(-c3nc(-c4ccccc4)nc(-c4ccccc4)n3)cc2)ccc1-c1ccccc1]

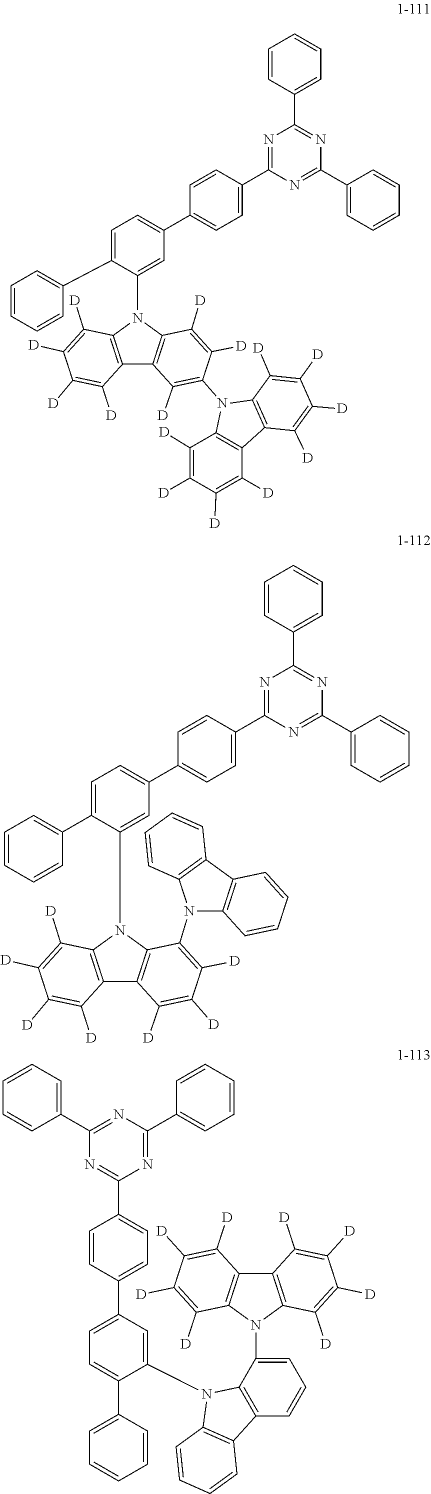

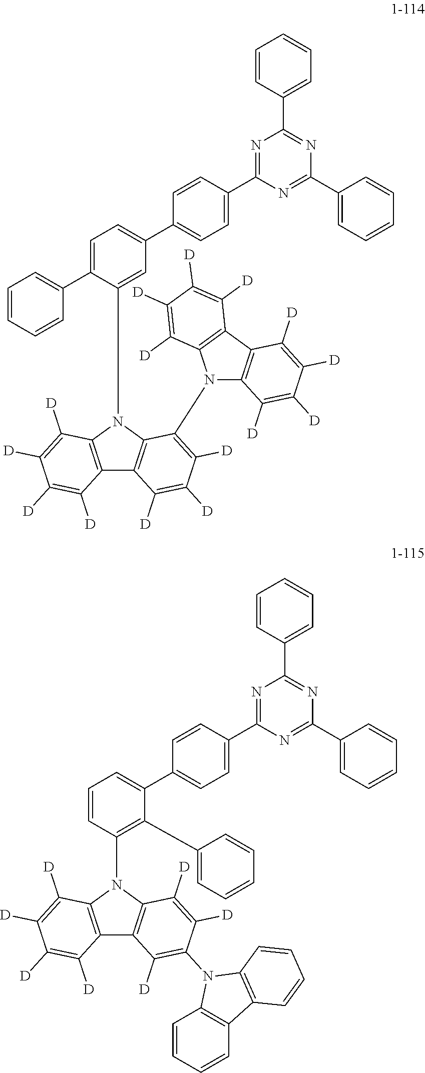 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccc(-c3nc(-c4ccccc4)nc(-c4ccccc4)n3)cc2)ccc1-c1ccccc1 ; [2H]c1c([2H])c([2H])c2c(c1[2H])c1c([2H])c(-n3c4ccccc4c4ccccc43)c([2H])c([2H])c1n2-c1cccc(-c2ccc(-c3nc(-c4ccccc4)nc(-c4ccccc4)n3)cc2)c1-c1ccccc1]

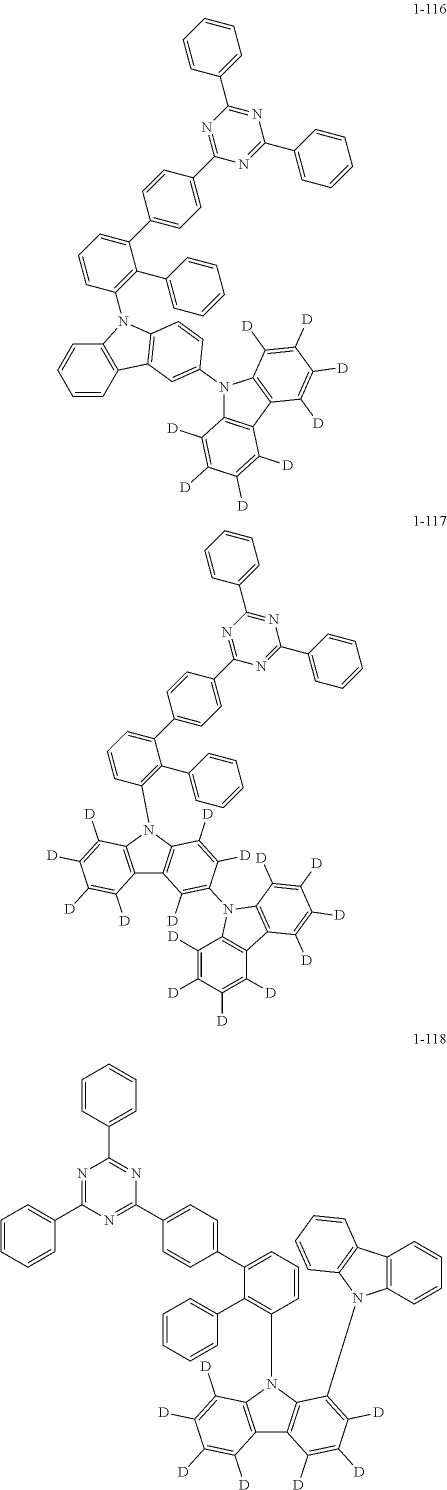

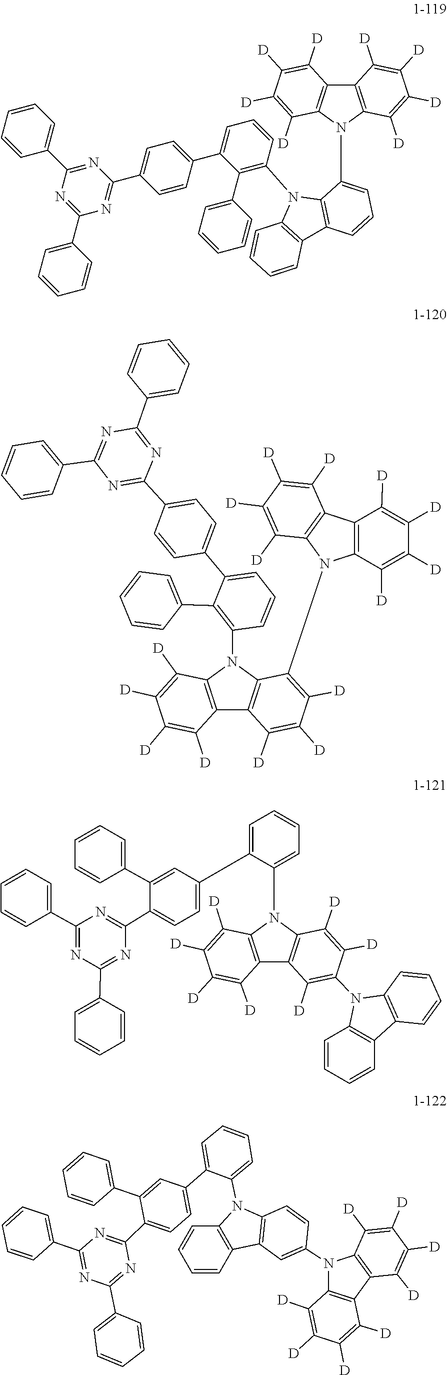

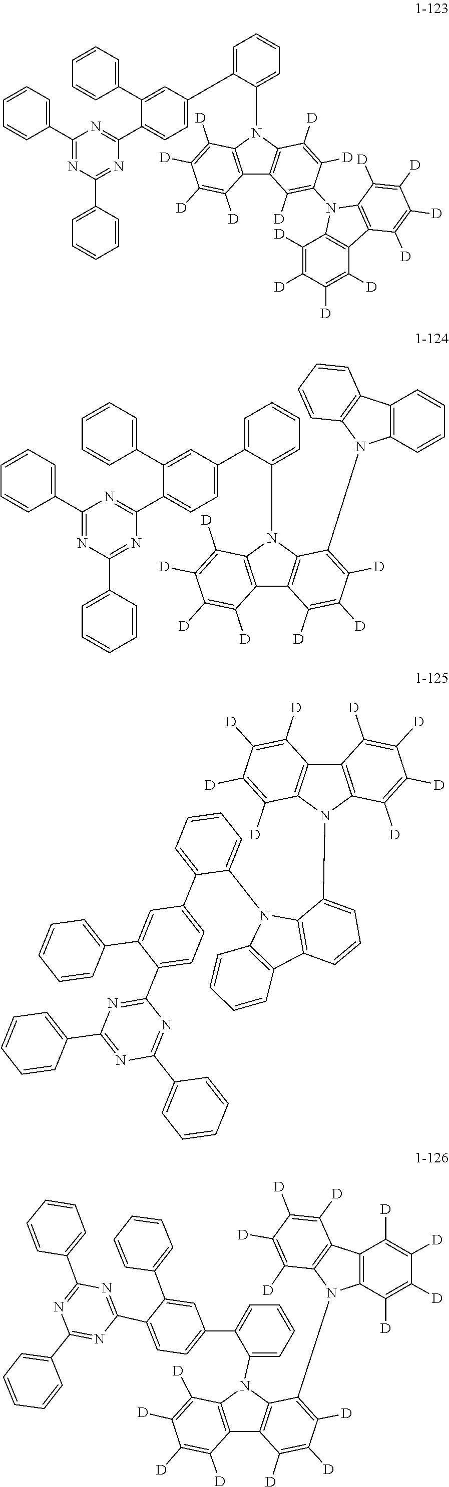

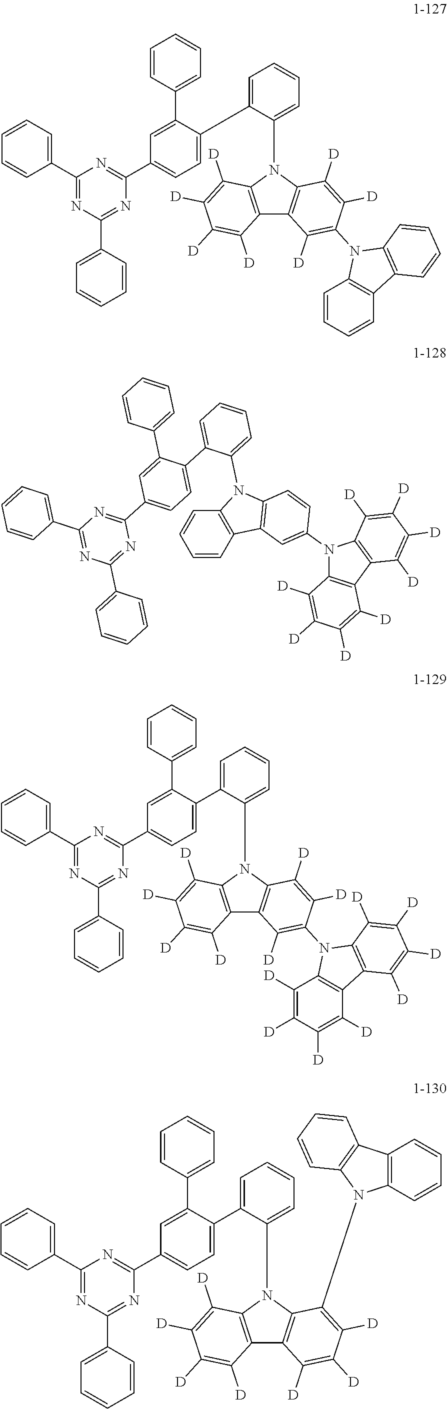

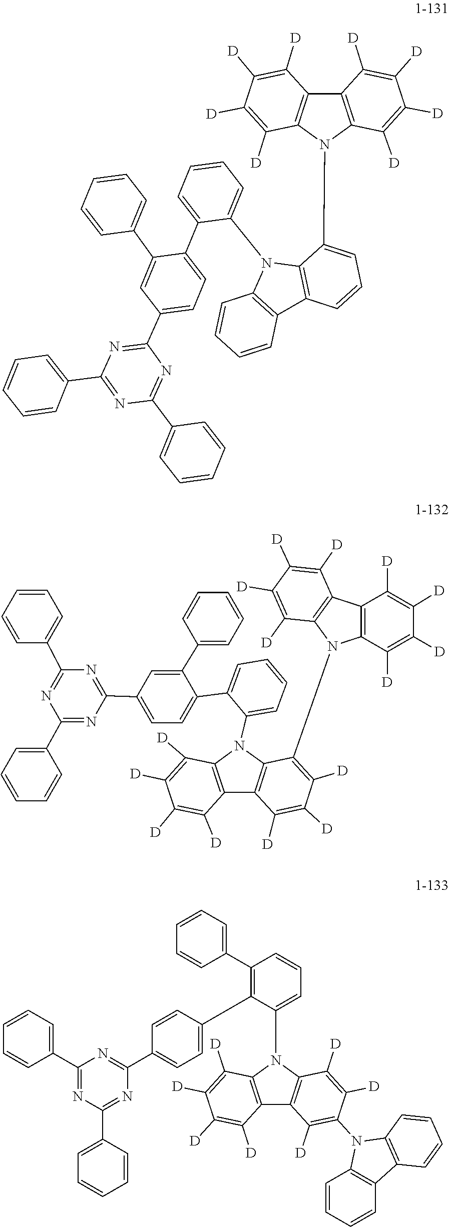 [SMILES: [2H]c1c([2H])c([2H])c2c(c1[2H])c1c([2H])c([2H])c([2H])c([2H])c1n2-c1cccc2c3ccccc3n(-c3ccccc3-c3ccc(-c4nc(-c5ccccc5)nc(-c5ccccc5)n4)cc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1ccc(-c2nc(-c3ccccc3)nc(-c3ccccc3)n2)cc1-c1ccccc1 ; [2H]c1c([2H])c([2H])c2c(c1[2H])c1c([2H])c(-n3c4ccccc4c4ccccc43)c([2H])c([2H])c1n2-c1cccc(-c2ccccc2)c1-c1ccc(-c2nc(-c3ccccc3)nc(-c3ccccc3)n2)cc1]

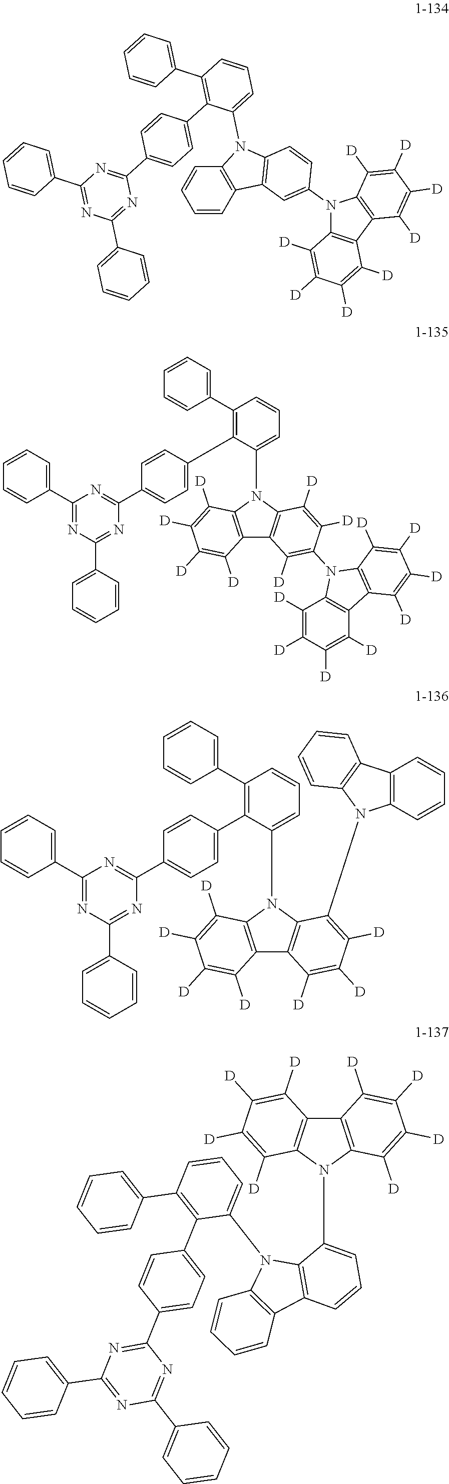

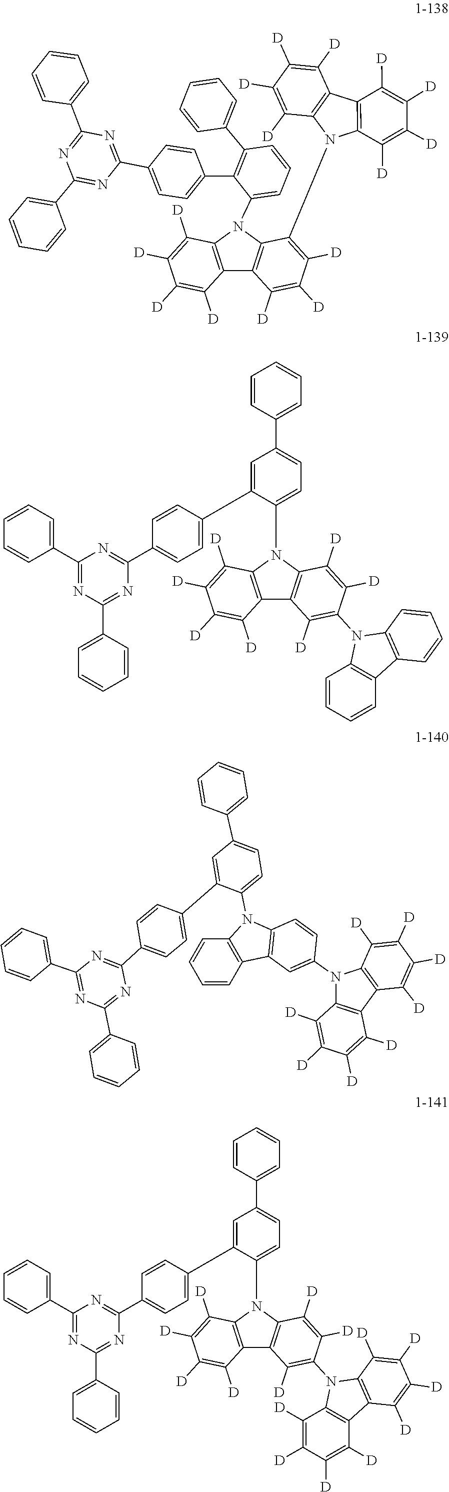

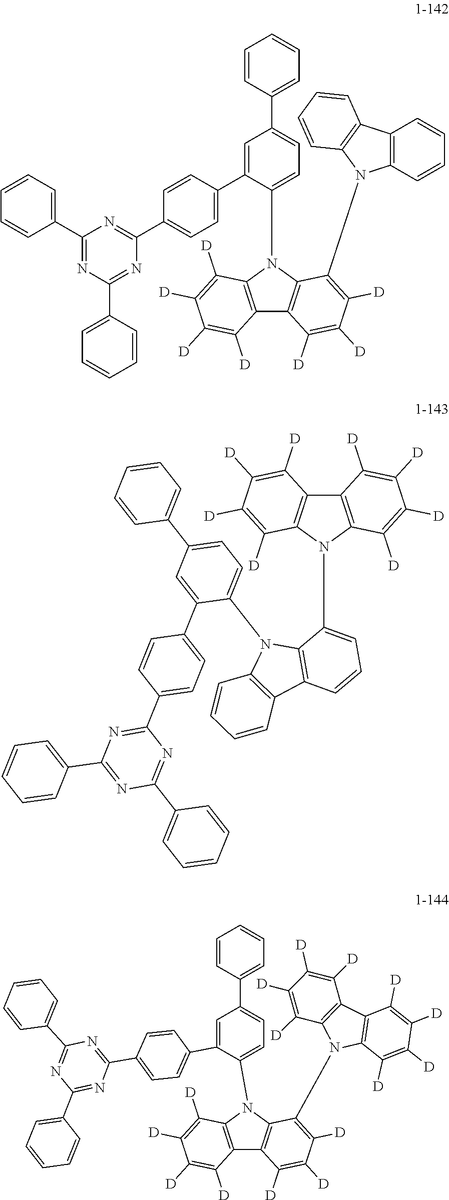 [SMILES: [2H]c1c([2H])c([2H])c2c(c1[2H])c1c([2H])c([2H])c([2H])c(-n3c4ccccc4c4ccccc43)c1n2-c1ccc(-c2ccccc2)cc1-c1ccc(-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1cccc2c3ccccc3n(-c3ccc(-c4ccccc4)cc3-c3ccc(-c4nc(-c5ccccc5)nc(-c5ccccc5)n4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)cc1-c1ccc(-c2nc(-c3ccccc3)nc(-c3ccccc3)n2)cc1]

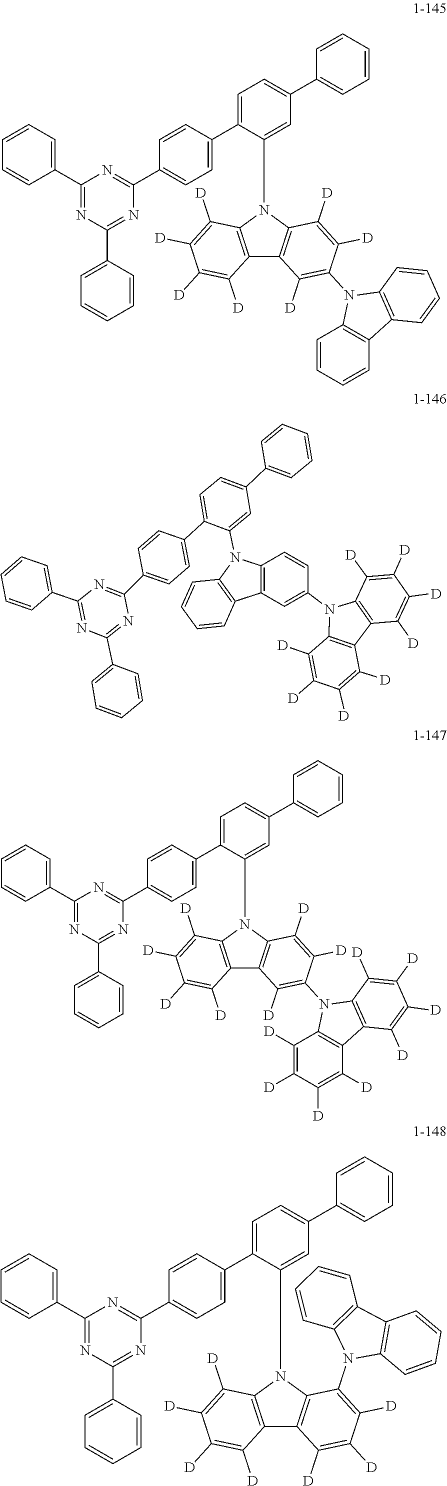

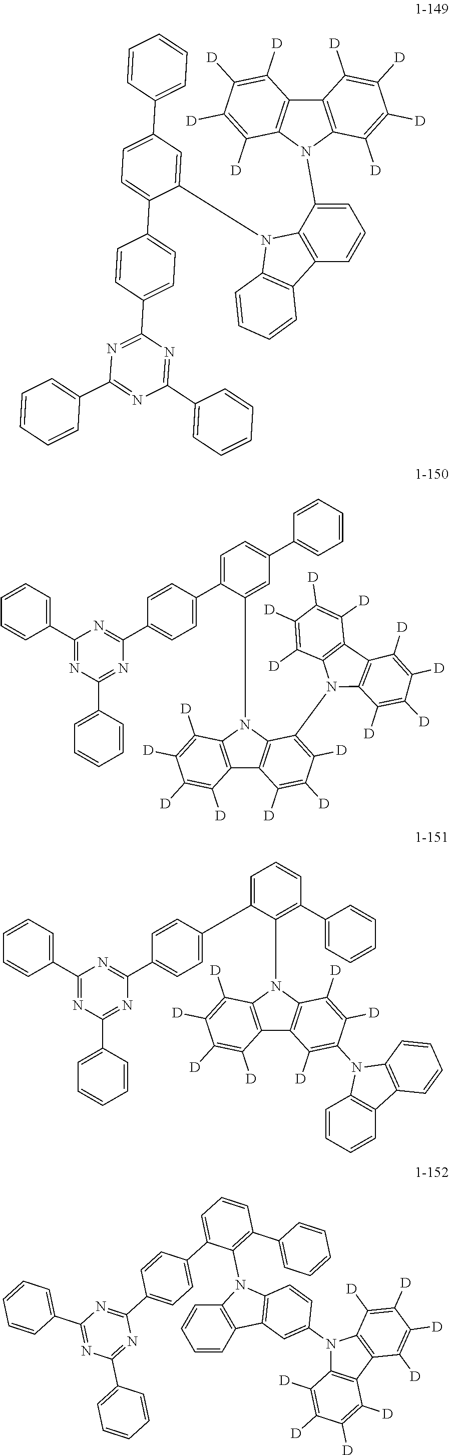

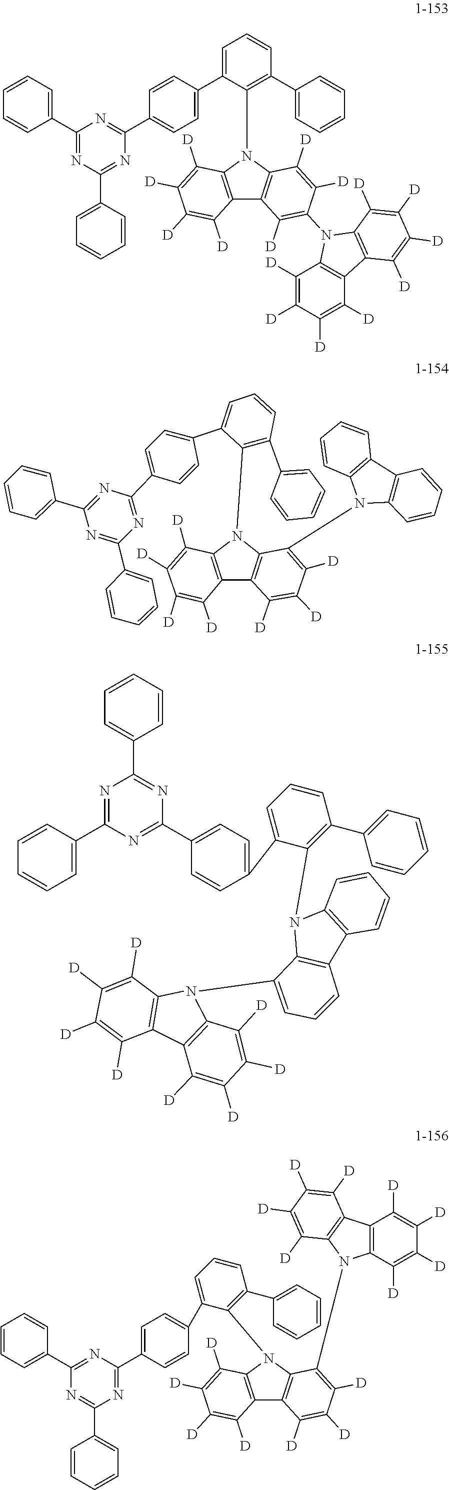

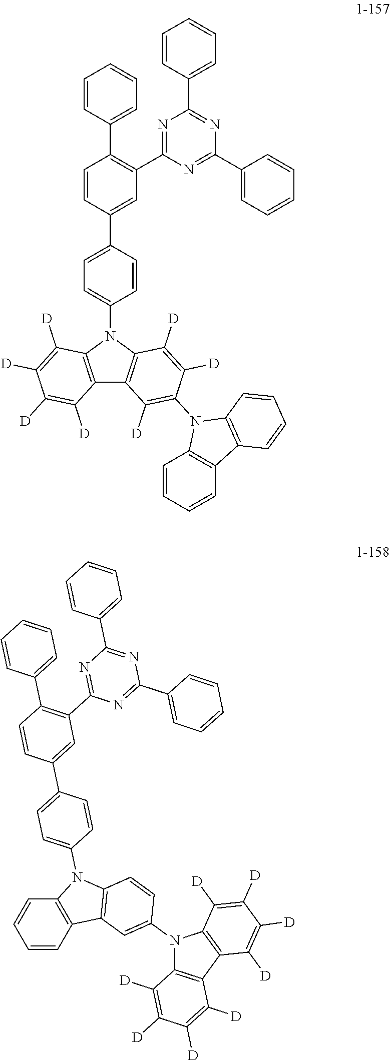 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3ccccc3)c(-c3nc(-c4ccccc4)nc(-c4ccccc4)n3)c2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3ccccc3)c(-c3nc(-c4ccccc4)nc(-c4ccccc4)n3)c2)cc1]

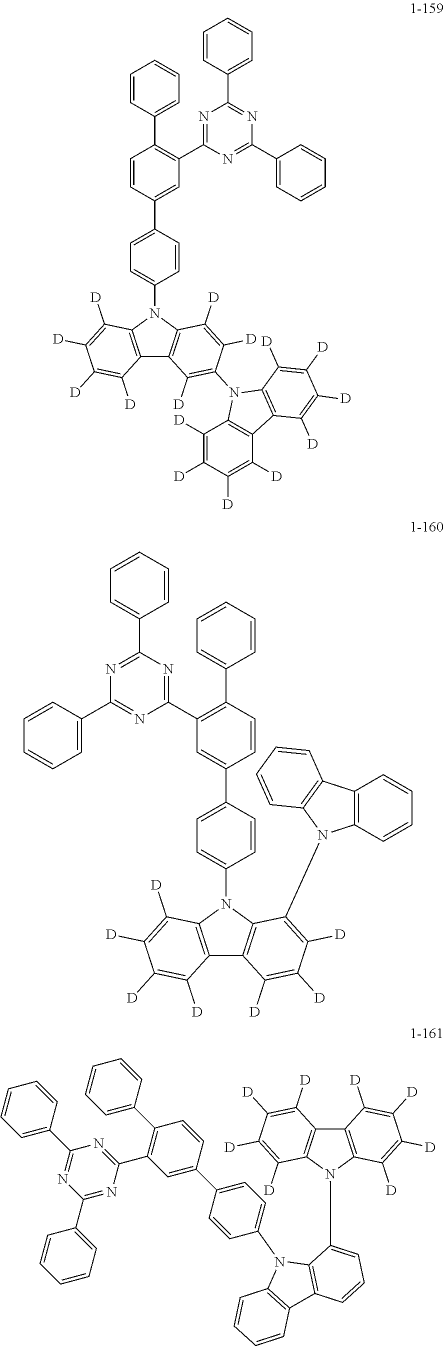

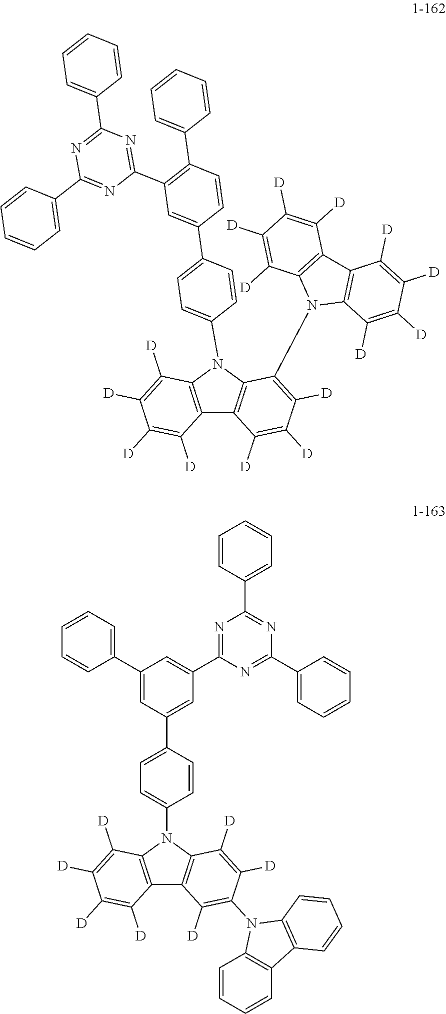 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3ccccc3)c(-c3nc(-c4ccccc4)nc(-c4ccccc4)n3)c2)cc1 ; [2H]c1c([2H])c([2H])c2c(c1[2H])c1c([2H])c(-n3c4ccccc4c4ccccc43)c([2H])c([2H])c1n2-c1ccc(-c2cc(-c3ccccc3)cc(-c3nc(-c4ccccc4)nc(-c4ccccc4)n3)c2)cc1]

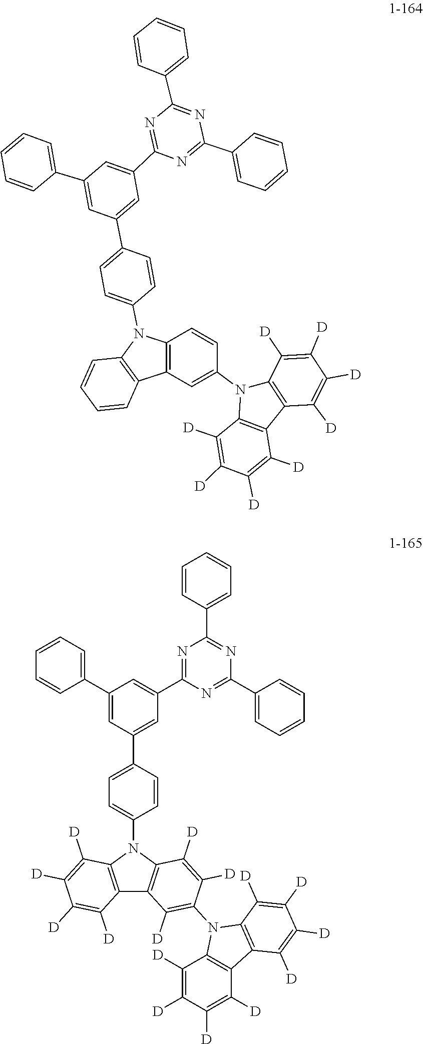 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2cc(-c3ccccc3)cc(-c3nc(-c4ccccc4)nc(-c4ccccc4)n3)c2)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cc(-c3ccccc3)cc(-c3nc(-c4ccccc4)nc(-c4ccccc4)n3)c2)cc1]

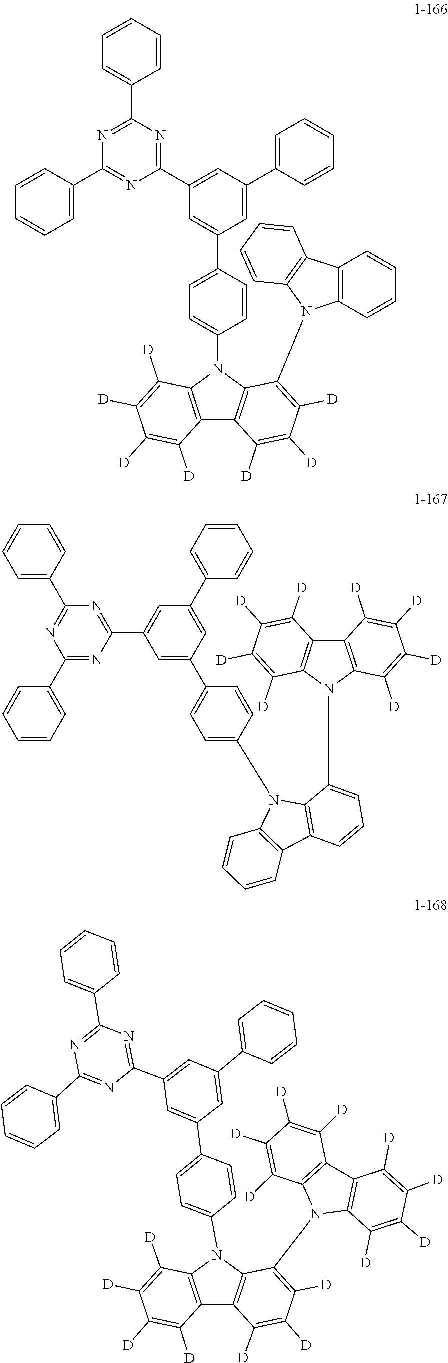 [SMILES: [2H]c1c([2H])c([2H])c2c(c1[2H])c1c([2H])c([2H])c([2H])c(-n3c4ccccc4c4ccccc43)c1n2-c1ccc(-c2cc(-c3ccccc3)cc(-c3nc(-c4ccccc4)nc(-c4ccccc4)n3)c2)cc1 ; [2H]c1c([2H])c([2H])c2c(c1[2H])c1c([2H])c([2H])c([2H])c([2H])c1n2-c1cccc2c3ccccc3n(-c3ccc(-c4cc(-c5ccccc5)cc(-c5nc(-c6ccccc6)nc(-c6ccccc6)n5)c4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2cc(-c3ccccc3)cc(-c3nc(-c4ccccc4)nc(-c4ccccc4)n3)c2)cc1]

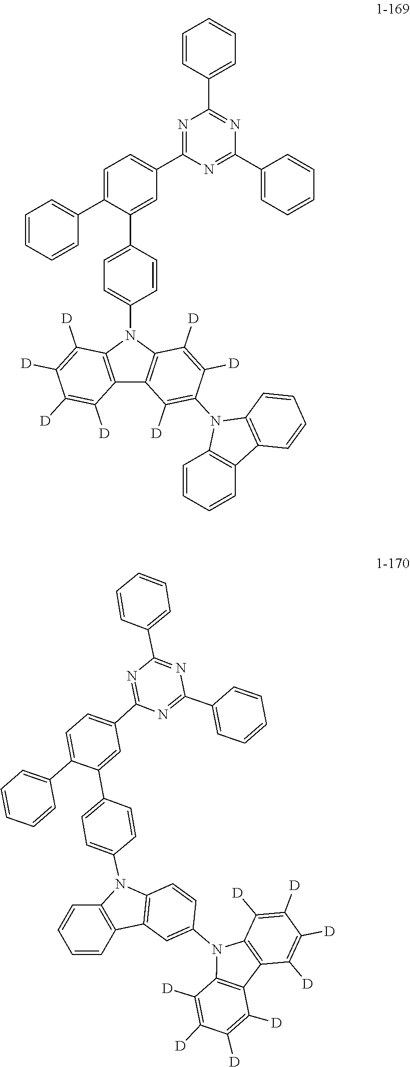 [SMILES: [2H]c1c([2H])c([2H])c2c(c1[2H])c1c([2H])c(-n3c4ccccc4c4ccccc43)c([2H])c([2H])c1n2-c1ccc(-c2cc(-c3nc(-c4ccccc4)nc(-c4ccccc4)n3)ccc2-c2ccccc2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2cc(-c3nc(-c4ccccc4)nc(-c4ccccc4)n3)ccc2-c2ccccc2)cc1]

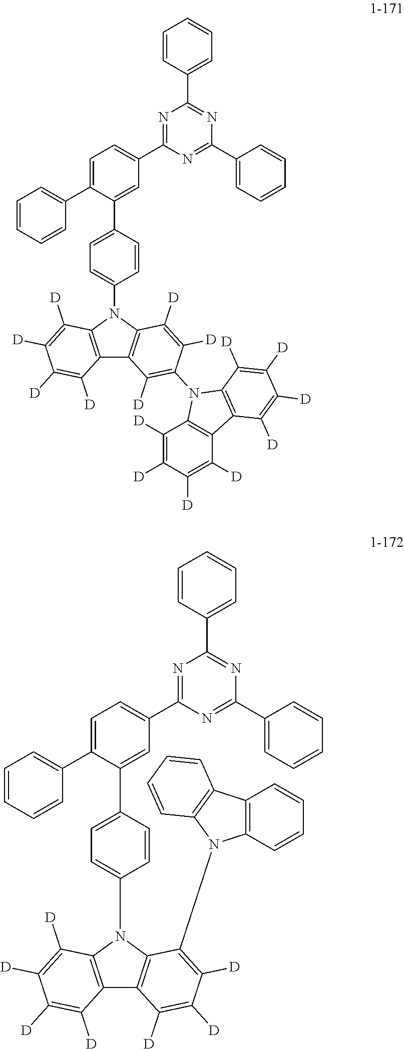 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cc(-c3nc(-c4ccccc4)nc(-c4ccccc4)n3)ccc2-c2ccccc2)cc1 ; [2H]c1c([2H])c([2H])c2c(c1[2H])c1c([2H])c([2H])c([2H])c(-n3c4ccccc4c4ccccc43)c1n2-c1ccc(-c2cc(-c3nc(-c4ccccc4)nc(-c4ccccc4)n3)ccc2-c2ccccc2)cc1]

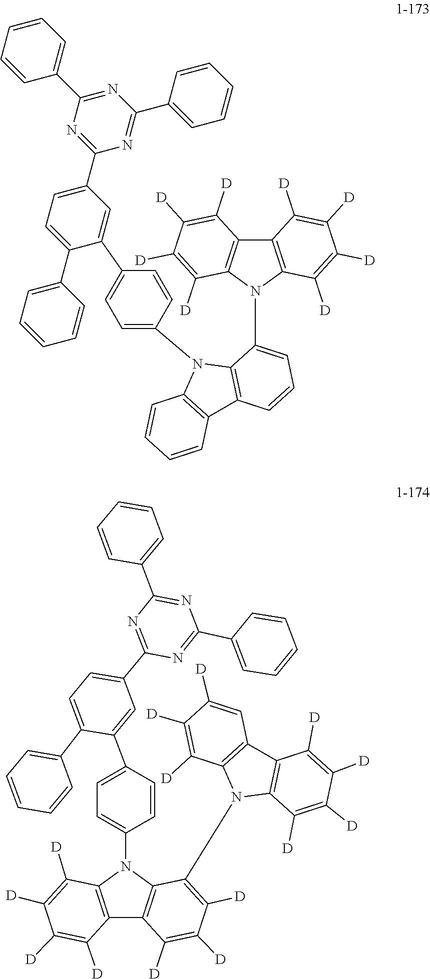 [SMILES: [2H]c1c([2H])c([2H])c2c(c1[2H])c1c([2H])c([2H])c([2H])c([2H])c1n2-c1cccc2c3ccccc3n(-c3ccc(-c4cc(-c5nc(-c6ccccc6)nc(-c6ccccc6)n5)ccc4-c4ccccc4)cc3)c12 ; [2H]c1cc2c3c([2H])c([2H])c([2H])c([2H])c3n(-c3c([2H])c([2H])c([2H])c4c5c([2H])c([2H])c([2H])c([2H])c5n(-c5ccc(-c6cc(-c7nc(-c8ccccc8)nc(-c8ccccc8)n7)ccc6-c6ccccc6)cc5)c34)c2c([2H])c1[2H]]

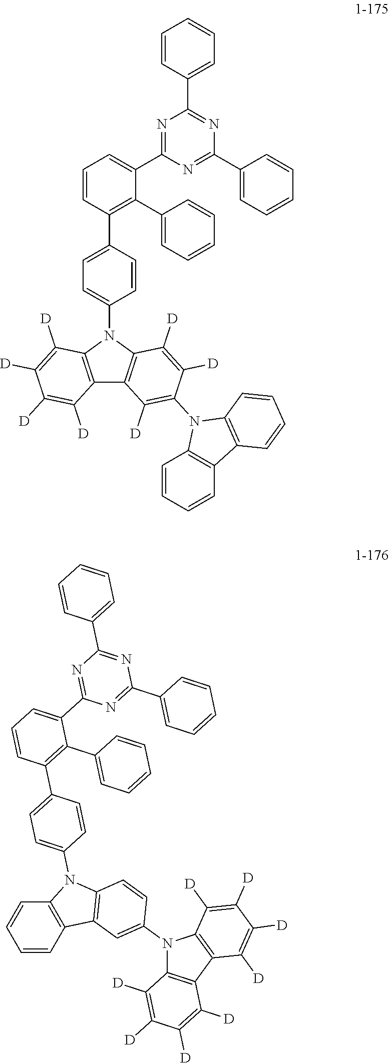 [SMILES: [2H]c1c([2H])c([2H])c2c(c1[2H])c1c([2H])c(-n3c4ccccc4c4ccccc43)c([2H])c([2H])c1n2-c1ccc(-c2cccc(-c3nc(-c4ccccc4)nc(-c4ccccc4)n3)c2-c2ccccc2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2cccc(-c3nc(-c4ccccc4)nc(-c4ccccc4)n3)c2-c2ccccc2)cc1]

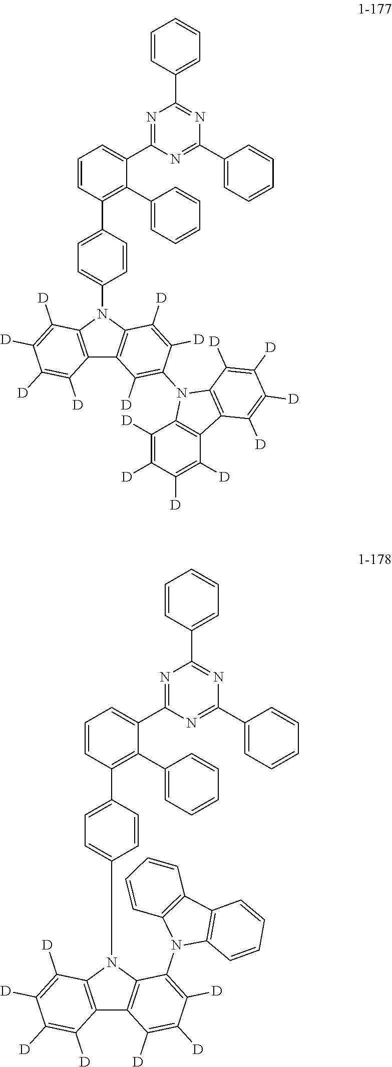 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cccc(-c3nc(-c4ccccc4)nc(-c4ccccc4)n3)c2-c2ccccc2)cc1 ; [2H]c1c([2H])c([2H])c2c(c1[2H])c1c([2H])c([2H])c([2H])c(-n3c4ccccc4c4ccccc43)c1n2-c1ccc(-c2cccc(-c3nc(-c4ccccc4)nc(-c4ccccc4)n3)c2-c2ccccc2)cc1]

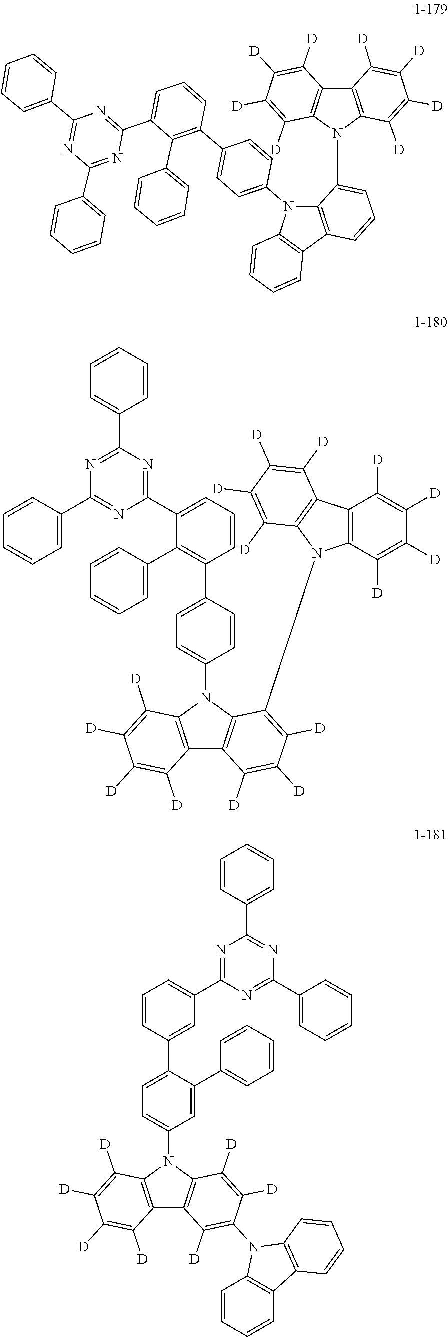

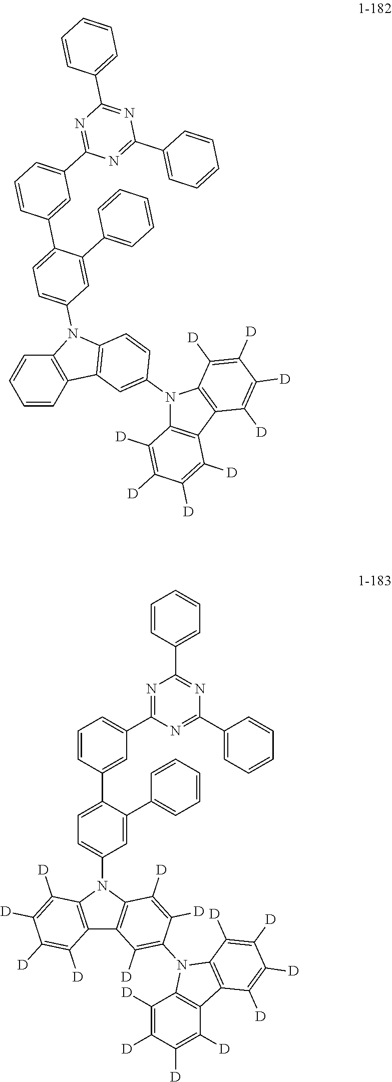 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2cccc(-c3nc(-c4ccccc4)nc(-c4ccccc4)n3)c2)c(-c2ccccc2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cccc(-c3nc(-c4ccccc4)nc(-c4ccccc4)n3)c2)c(-c2ccccc2)c1]

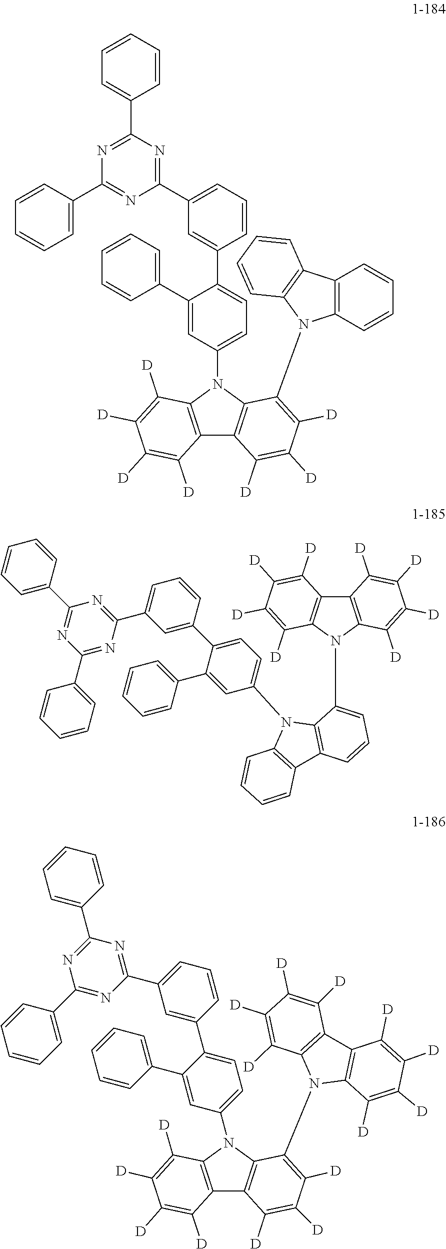 [SMILES: [2H]c1c([2H])c([2H])c2c(c1[2H])c1c([2H])c([2H])c([2H])c(-n3c4ccccc4c4ccccc43)c1n2-c1ccc(-c2cccc(-c3nc(-c4ccccc4)nc(-c4ccccc4)n3)c2)c(-c2ccccc2)c1 ; [2H]c1c([2H])c([2H])c2c(c1[2H])c1c([2H])c([2H])c([2H])c([2H])c1n2-c1cccc2c3ccccc3n(-c3ccc(-c4cccc(-c5nc(-c6ccccc6)nc(-c6ccccc6)n5)c4)c(-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2cccc(-c3nc(-c4ccccc4)nc(-c4ccccc4)n3)c2)c(-c2ccccc2)c1]

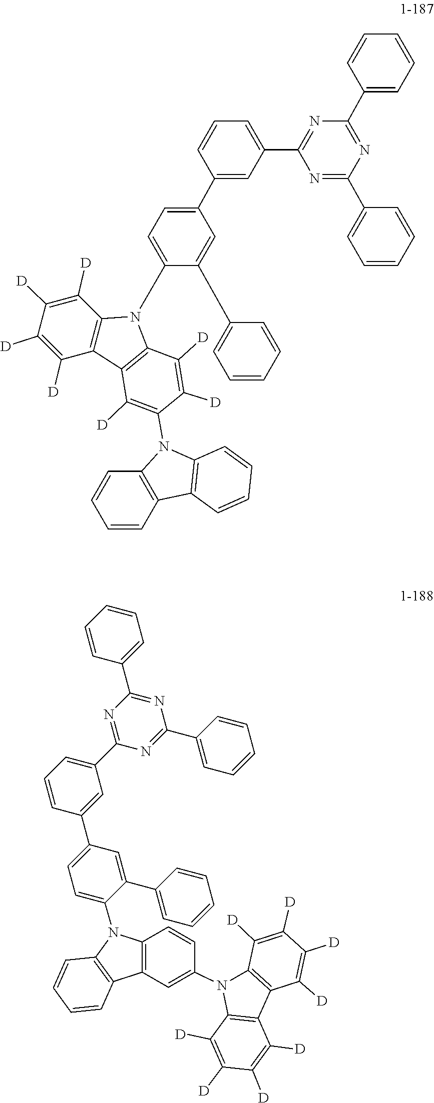 [SMILES: [2H]c1c([2H])c([2H])c2c(c1[2H])c1c([2H])c(-n3c4ccccc4c4ccccc43)c([2H])c([2H])c1n2-c1ccc(-c2cccc(-c3nc(-c4ccccc4)nc(-c4ccccc4)n3)c2)cc1-c1ccccc1 ; [2H]c1c([2H])c([2H])c2c(c1[2H])c1c([2H])c([2H])c([2H])c([2H])c1n2-c1ccc2c(c1)c1ccccc1n2-c1ccc(-c2cccc(-c3nc(-c4ccccc4)nc(-c4ccccc4)n3)c2)cc1-c1ccccc1]

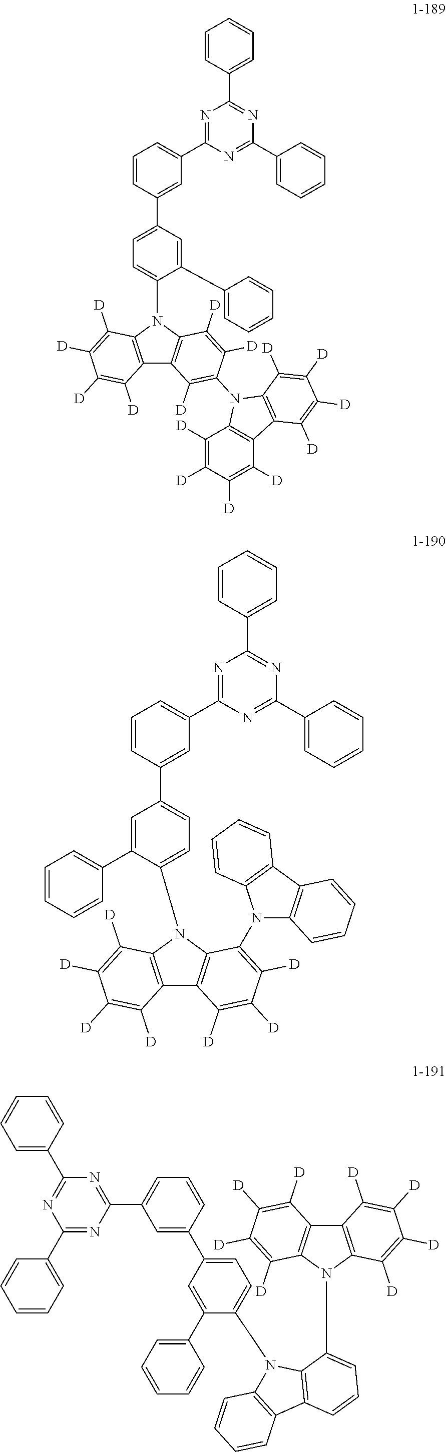 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cccc(-c3nc(-c4ccccc4)nc(-c4ccccc4)n3)c2)cc1-c1ccccc1 ; [2H]c1c([2H])c([2H])c2c(c1[2H])c1c([2H])c([2H])c([2H])c(-n3c4ccccc4c4ccccc43)c1n2-c1ccc(-c2cccc(-c3nc(-c4ccccc4)nc(-c4ccccc4)n3)c2)cc1-c1ccccc1 ; [2H]c1c([2H])c([2H])c2c(c1[2H])c1c([2H])c([2H])c([2H])c([2H])c1n2-c1cccc2c3ccccc3n(-c3ccc(-c4cccc(-c5nc(-c6ccccc6)nc(-c6ccccc6)n5)c4)cc3-c3ccccc3)c12]

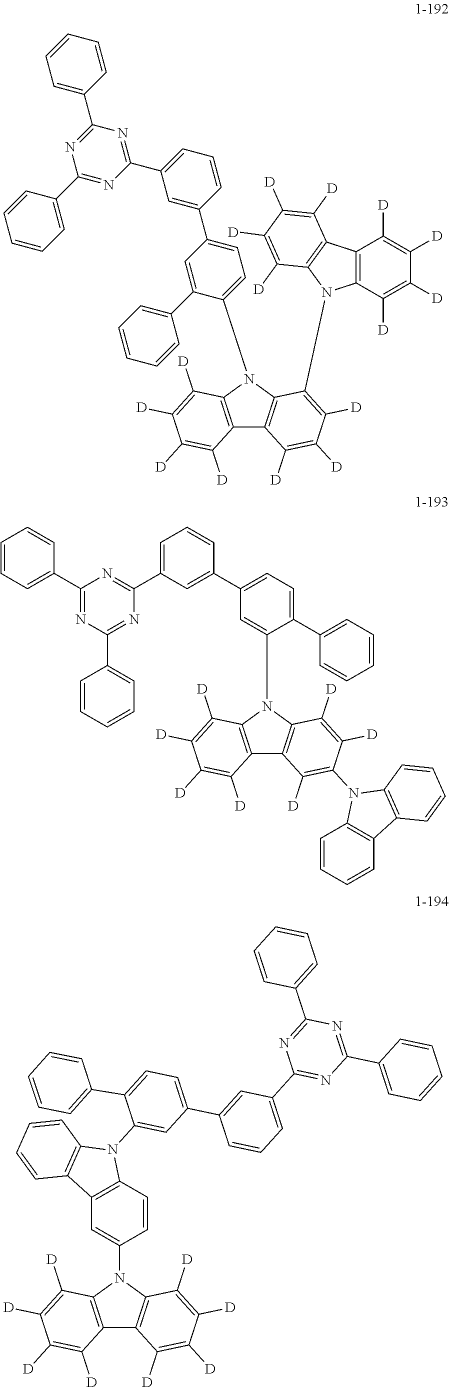 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2cccc(-c3nc(-c4ccccc4)nc(-c4ccccc4)n3)c2)cc1-c1ccccc1 ; [2H]c1c([2H])c([2H])c2c(c1[2H])c1c([2H])c(-n3c4ccccc4c4ccccc43)c([2H])c([2H])c1n2-c1cc(-c2cccc(-c3nc(-c4ccccc4)nc(-c4ccccc4)n3)c2)ccc1-c1ccccc1 ; [2H]c1c([2H])c([2H])c2c(c1[2H])c1c([2H])c([2H])c([2H])c([2H])c1n2-c1ccc2c(c1)c1ccccc1n2-c1cc(-c2cccc(-c3nc(-c4ccccc4)nc(-c4ccccc4)n3)c2)ccc1-c1ccccc1]

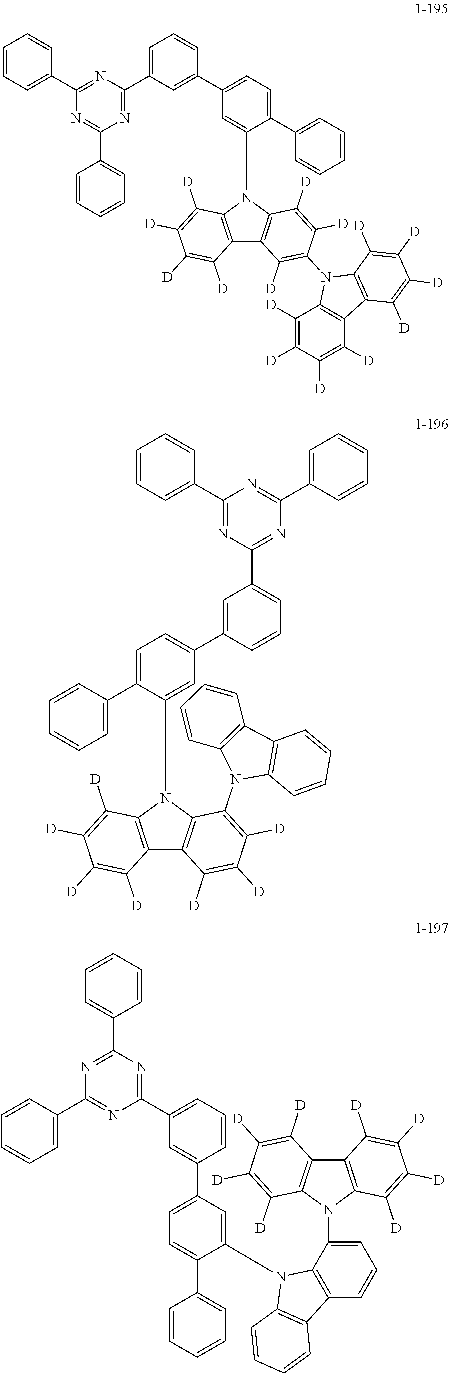 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2cccc(-c3nc(-c4ccccc4)nc(-c4ccccc4)n3)c2)ccc1-c1ccccc1 ; [2H]c1c([2H])c([2H])c2c(c1[2H])c1c([2H])c([2H])c([2H])c(-n3c4ccccc4c4ccccc43)c1n2-c1cc(-c2cccc(-c3nc(-c4ccccc4)nc(-c4ccccc4)n3)c2)ccc1-c1ccccc1 ; [2H]c1c([2H])c([2H])c2c(c1[2H])c1c([2H])c([2H])c([2H])c([2H])c1n2-c1cccc2c3ccccc3n(-c3cc(-c4cccc(-c5nc(-c6ccccc6)nc(-c6ccccc6)n5)c4)ccc3-c3ccccc3)c12]

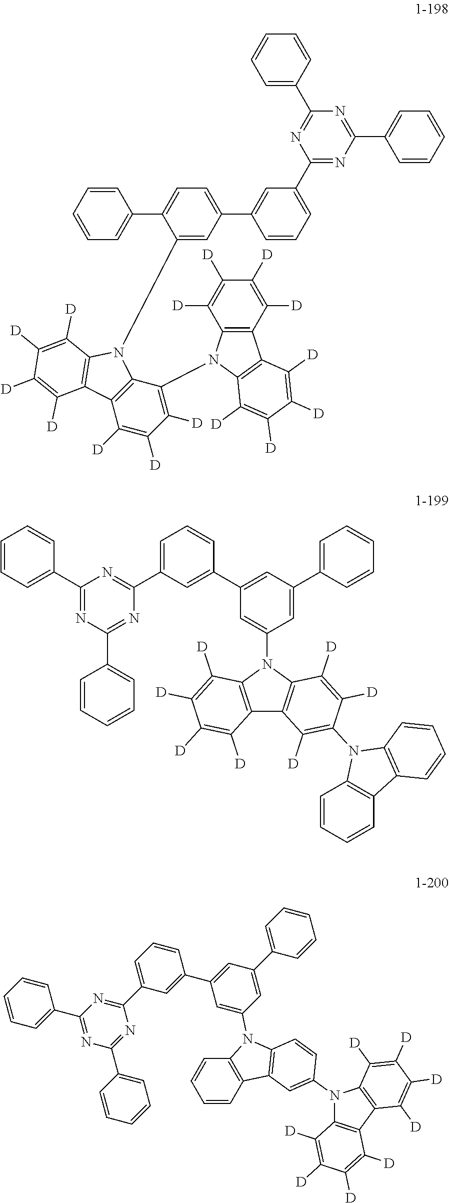 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2cccc(-c3nc(-c4ccccc4)nc(-c4ccccc4)n3)c2)ccc1-c1ccccc1 ; [2H]c1c([2H])c([2H])c2c(c1[2H])c1c([2H])c(-n3c4ccccc4c4ccccc43)c([2H])c([2H])c1n2-c1cc(-c2ccccc2)cc(-c2cccc(-c3nc(-c4ccccc4)nc(-c4ccccc4)n3)c2)c1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2)cc(-c2cccc(-c3nc(-c4ccccc4)nc(-c4ccccc4)n3)c2)c1]

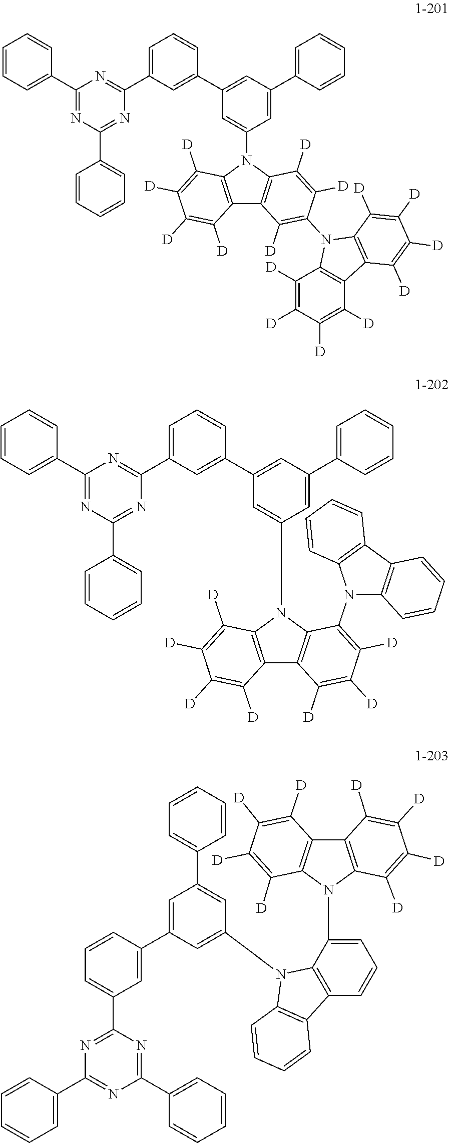 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)cc(-c2cccc(-c3nc(-c4ccccc4)nc(-c4ccccc4)n3)c2)c1 ; [2H]c1c([2H])c([2H])c2c(c1[2H])c1c([2H])c([2H])c([2H])c(-n3c4ccccc4c4ccccc43)c1n2-c1cc(-c2ccccc2)cc(-c2cccc(-c3nc(-c4ccccc4)nc(-c4ccccc4)n3)c2)c1 ; [2H]c1c([2H])c([2H])c2c(c1[2H])c1c([2H])c([2H])c([2H])c([2H])c1n2-c1cccc2c3ccccc3n(-c3cc(-c4ccccc4)cc(-c4cccc(-c5nc(-c6ccccc6)nc(-c6ccccc6)n5)c4)c3)c12]

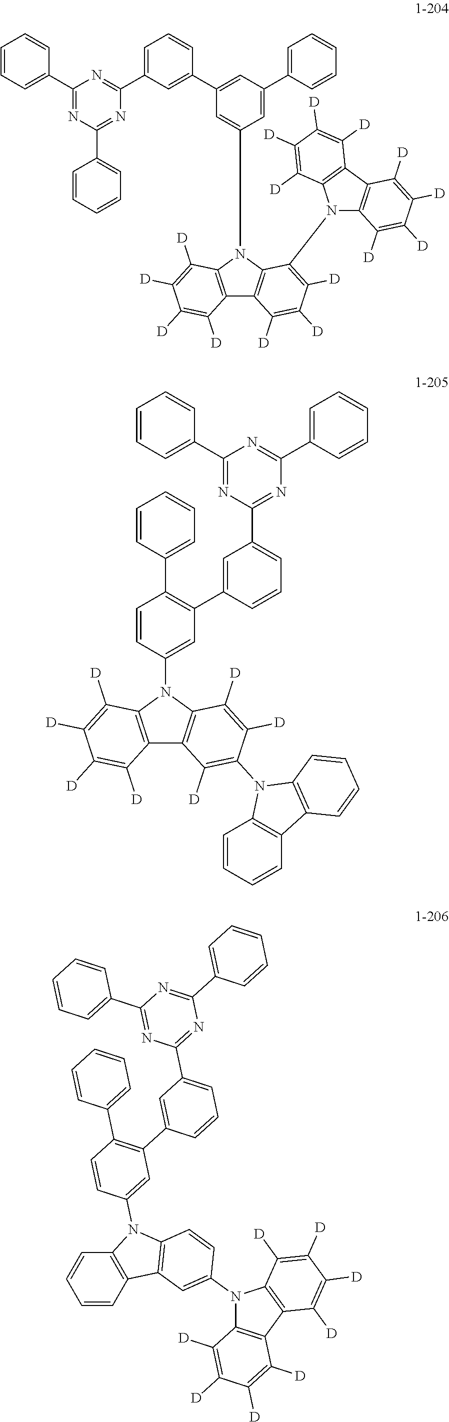

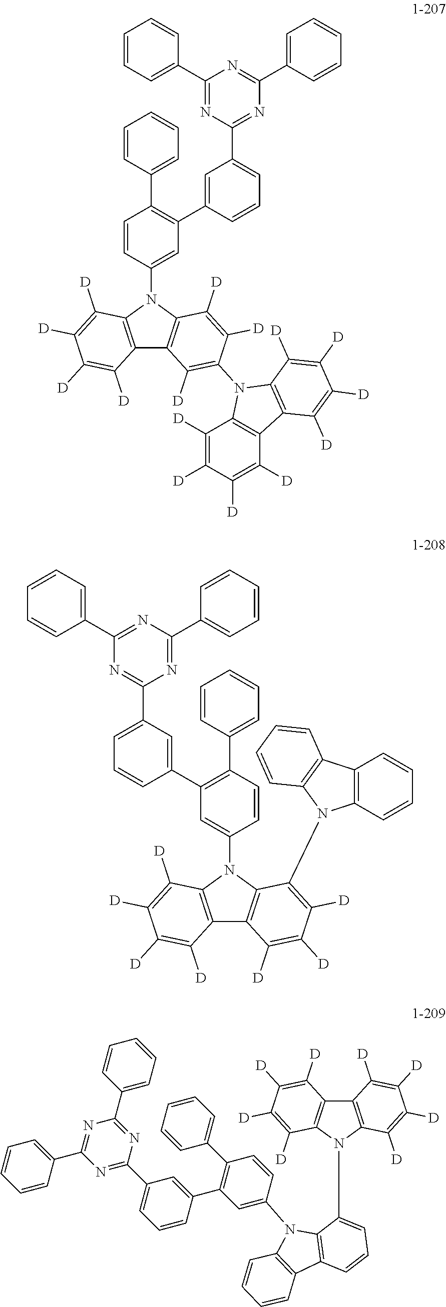 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)c(-c2cccc(-c3nc(-c4ccccc4)nc(-c4ccccc4)n3)c2)c1 ; [2H]c1c([2H])c([2H])c2c(c1[2H])c1c([2H])c([2H])c([2H])c(-n3c4ccccc4c4ccccc43)c1n2-c1ccc(-c2ccccc2)c(-c2cccc(-c3nc(-c4ccccc4)nc(-c4ccccc4)n3)c2)c1 ; [2H]c1c([2H])c([2H])c2c(c1[2H])c1c([2H])c([2H])c([2H])c([2H])c1n2-c1cccc2c3ccccc3n(-c3ccc(-c4ccccc4)c(-c4cccc(-c5nc(-c6ccccc6)nc(-c6ccccc6)n5)c4)c3)c12]

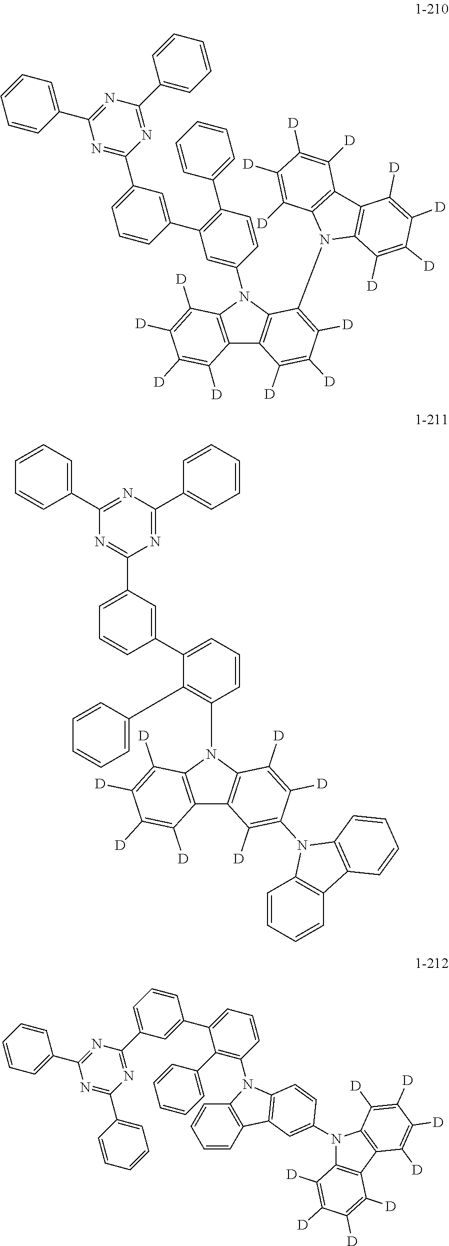 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)c(-c2cccc(-c3nc(-c4ccccc4)nc(-c4ccccc4)n3)c2)c1 ; [2H]c1c([2H])c([2H])c2c(c1[2H])c1c([2H])c(-n3c4ccccc4c4ccccc43)c([2H])c([2H])c1n2-c1cccc(-c2cccc(-c3nc(-c4ccccc4)nc(-c4ccccc4)n3)c2)c1-c1ccccc1 ; [2H]c1c([2H])c([2H])c2c(c1[2H])c1c([2H])c([2H])c([2H])c([2H])c1n2-c1ccc2c(c1)c1ccccc1n2-c1cccc(-c2cccc(-c3nc(-c4ccccc4)nc(-c4ccccc4)n3)c2)c1-c1ccccc1]

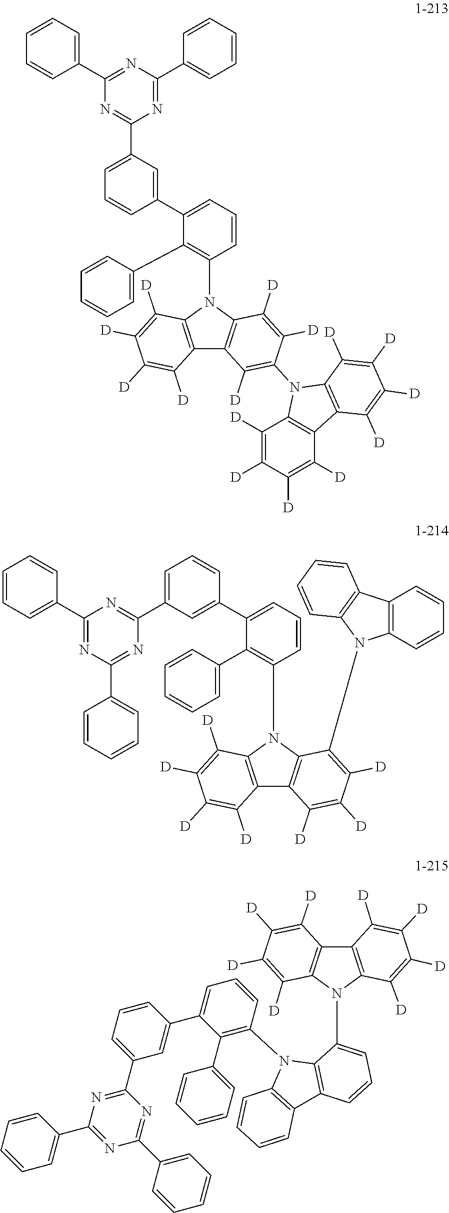

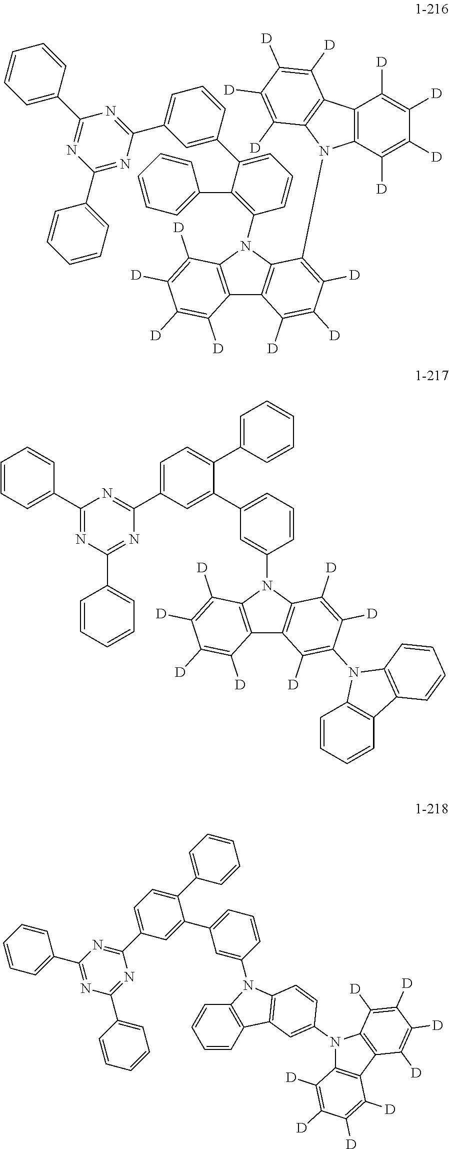

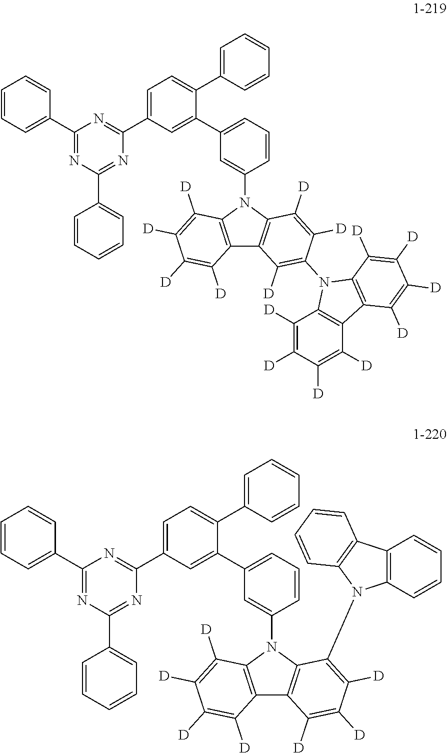 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cc(-c3nc(-c4ccccc4)nc(-c4ccccc4)n3)ccc2-c2ccccc2)c1 ; [2H]c1c([2H])c([2H])c2c(c1[2H])c1c([2H])c([2H])c([2H])c(-n3c4ccccc4c4ccccc43)c1n2-c1cccc(-c2cc(-c3nc(-c4ccccc4)nc(-c4ccccc4)n3)ccc2-c2ccccc2)c1]

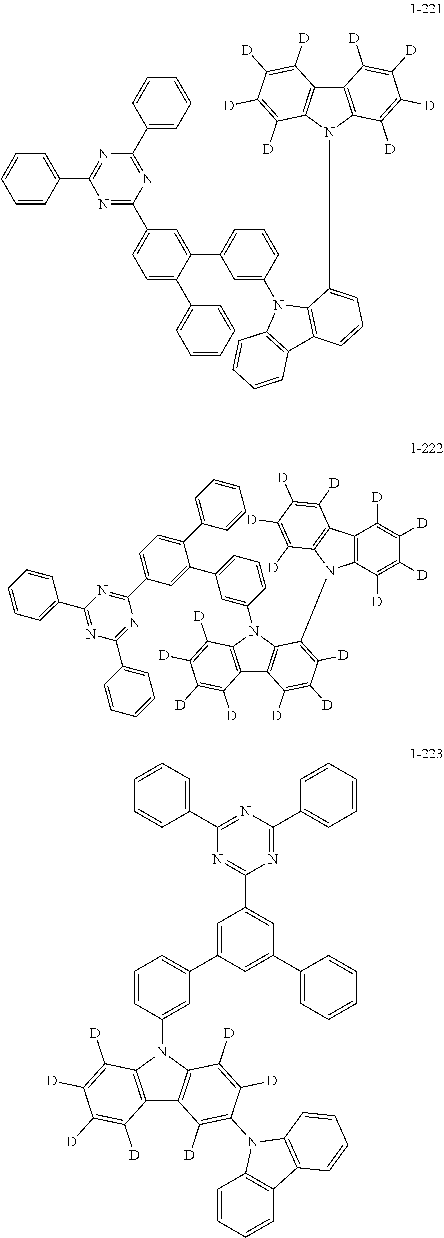 [SMILES: [2H]c1c([2H])c([2H])c2c(c1[2H])c1c([2H])c([2H])c([2H])c([2H])c1n2-c1cccc2c3ccccc3n(-c3cccc(-c4cc(-c5nc(-c6ccccc6)nc(-c6ccccc6)n5)ccc4-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cc(-c3nc(-c4ccccc4)nc(-c4ccccc4)n3)ccc2-c2ccccc2)c1 ; [2H]c1c([2H])c([2H])c2c(c1[2H])c1c([2H])c(-n3c4ccccc4c4ccccc43)c([2H])c([2H])c1n2-c1cccc(-c2cc(-c3ccccc3)cc(-c3nc(-c4ccccc4)nc(-c4ccccc4)n3)c2)c1]

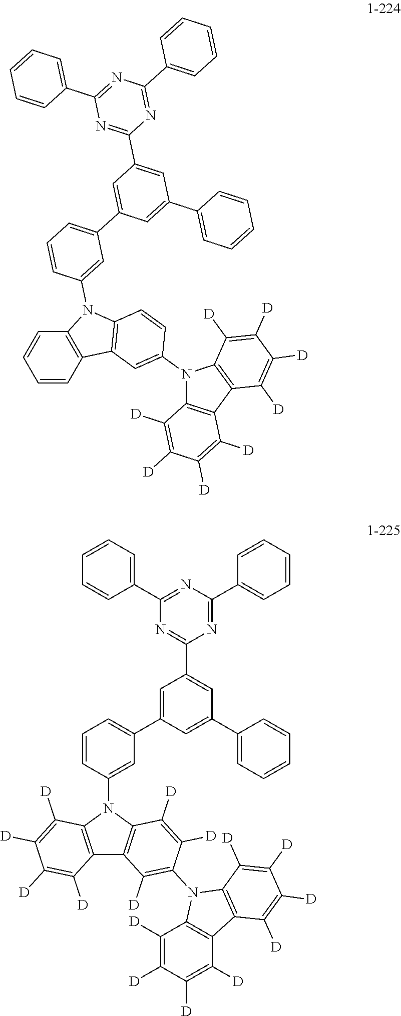 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2cc(-c3ccccc3)cc(-c3nc(-c4ccccc4)nc(-c4ccccc4)n3)c2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cc(-c3ccccc3)cc(-c3nc(-c4ccccc4)nc(-c4ccccc4)n3)c2)c1]

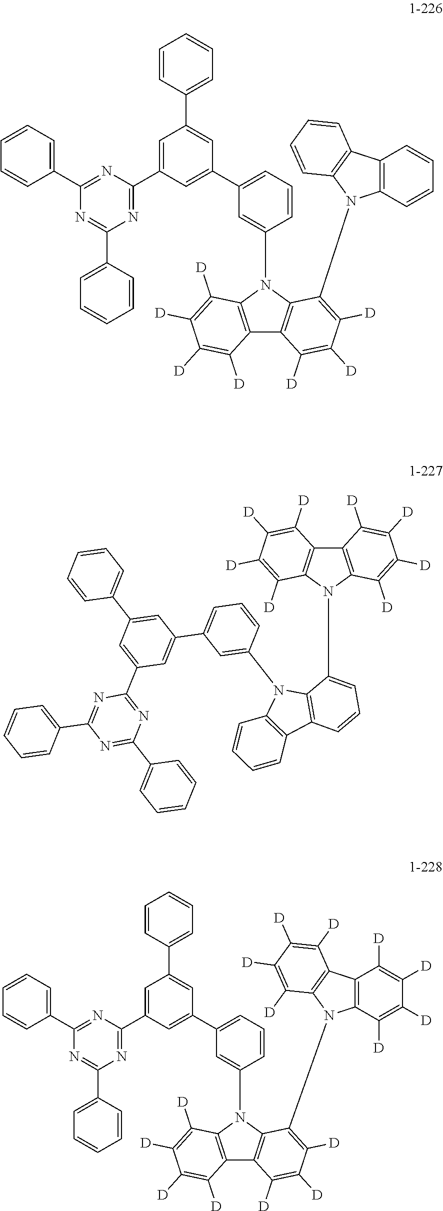 [SMILES: [2H]c1c([2H])c([2H])c2c(c1[2H])c1c([2H])c([2H])c([2H])c(-n3c4ccccc4c4ccccc43)c1n2-c1cccc(-c2cc(-c3ccccc3)cc(-c3nc(-c4ccccc4)nc(-c4ccccc4)n3)c2)c1 ; [2H]c1c([2H])c([2H])c2c(c1[2H])c1c([2H])c([2H])c([2H])c([2H])c1n2-c1cccc2c3ccccc3n(-c3cccc(-c4cc(-c5ccccc5)cc(-c5nc(-c6ccccc6)nc(-c6ccccc6)n5)c4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cc(-c3ccccc3)cc(-c3nc(-c4ccccc4)nc(-c4ccccc4)n3)c2)c1]

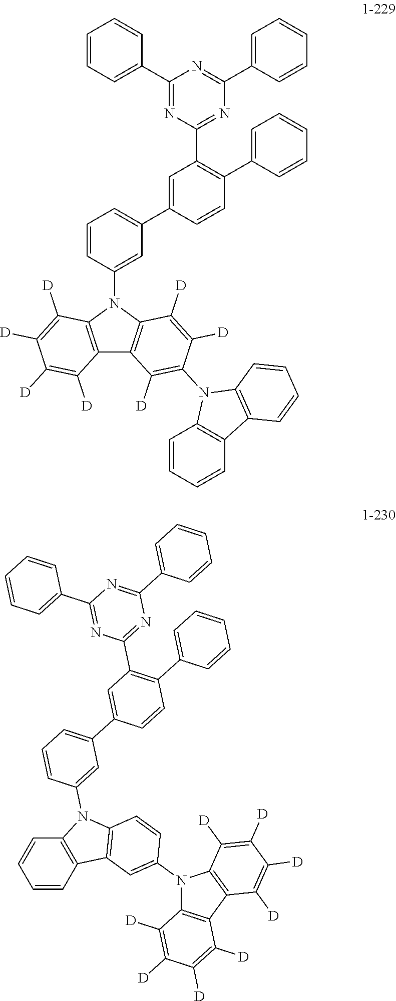 [SMILES: [2H]c1c([2H])c([2H])c2c(c1[2H])c1c([2H])c(-n3c4ccccc4c4ccccc43)c([2H])c([2H])c1n2-c1cccc(-c2ccc(-c3ccccc3)c(-c3nc(-c4ccccc4)nc(-c4ccccc4)n3)c2)c1 ; [2H]c1c([2H])c([2H])c2c(c1[2H])c1c([2H])c([2H])c([2H])c([2H])c1n2-c1ccc2c(c1)c1ccccc1n2-c1cccc(-c2ccc(-c3ccccc3)c(-c3nc(-c4ccccc4)nc(-c4ccccc4)n3)c2)c1]

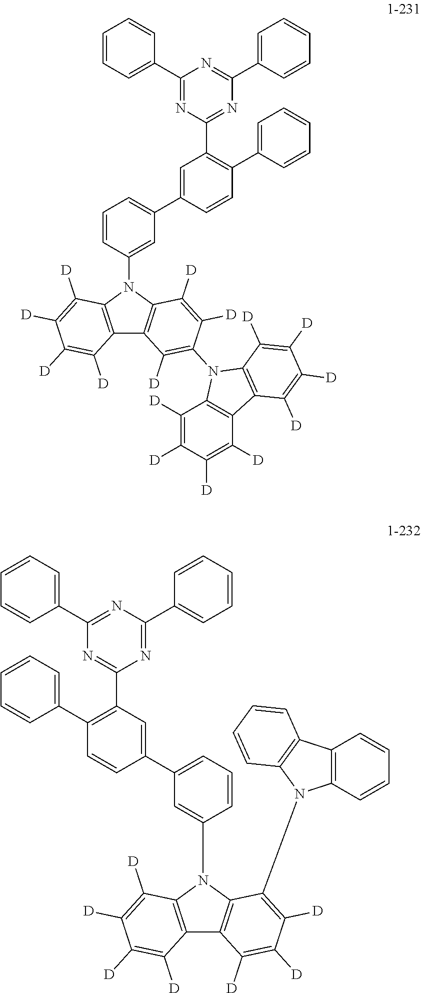 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccc(-c3ccccc3)c(-c3nc(-c4ccccc4)nc(-c4ccccc4)n3)c2)c1 ; [2H]c1c([2H])c([2H])c2c(c1[2H])c1c([2H])c([2H])c([2H])c(-n3c4ccccc4c4ccccc43)c1n2-c1cccc(-c2ccc(-c3ccccc3)c(-c3nc(-c4ccccc4)nc(-c4ccccc4)n3)c2)c1]

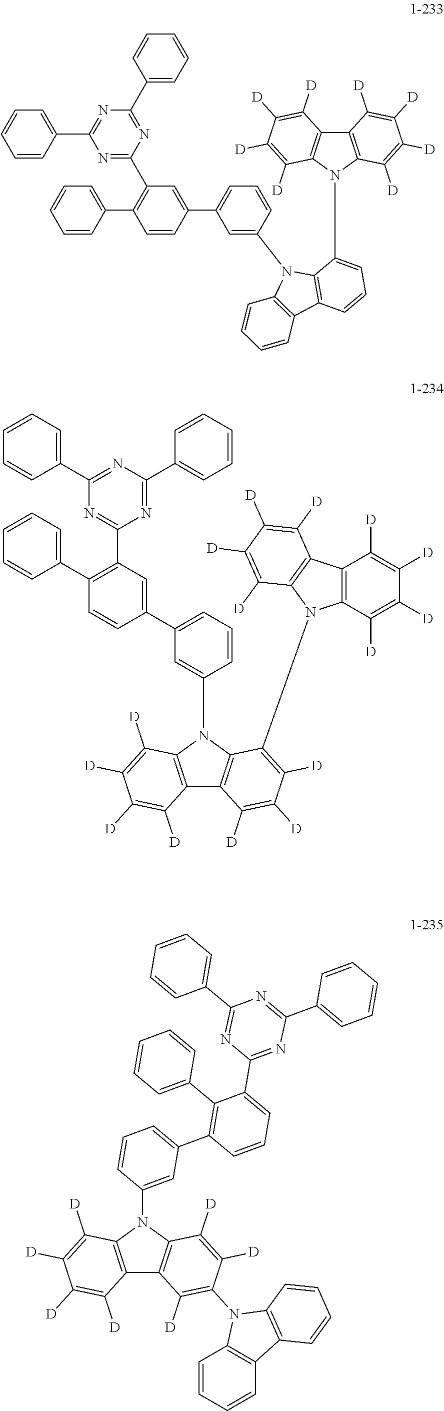 [SMILES: [2H]c1c([2H])c([2H])c2c(c1[2H])c1c([2H])c([2H])c([2H])c([2H])c1n2-c1cccc2c3ccccc3n(-c3cccc(-c4ccc(-c5ccccc5)c(-c5nc(-c6ccccc6)nc(-c6ccccc6)n5)c4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccc(-c3ccccc3)c(-c3nc(-c4ccccc4)nc(-c4ccccc4)n3)c2)c1 ; [2H]c1c([2H])c([2H])c2c(c1[2H])c1c([2H])c(-n3c4ccccc4c4ccccc43)c([2H])c([2H])c1n2-c1cccc(-c2cccc(-c3nc(-c4ccccc4)nc(-c4ccccc4)n3)c2-c2ccccc2)c1]

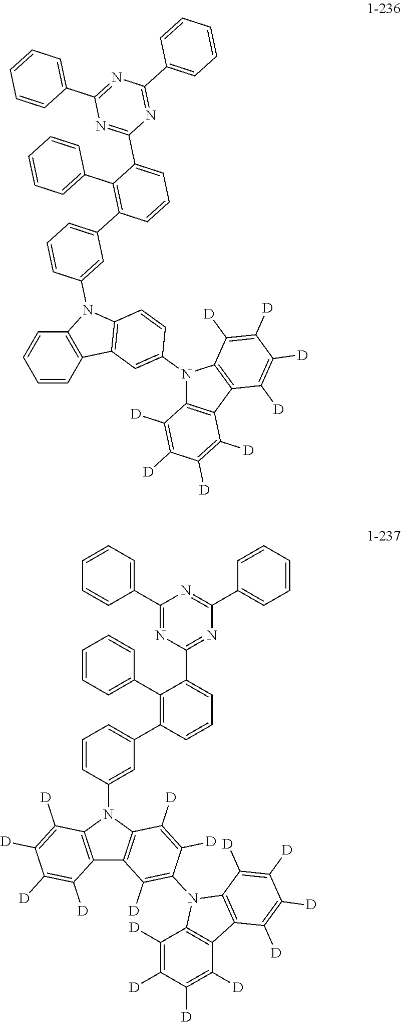 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2cccc(-c3nc(-c4ccccc4)nc(-c4ccccc4)n3)c2-c2ccccc2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cccc(-c3nc(-c4ccccc4)nc(-c4ccccc4)n3)c2-c2ccccc2)c1]

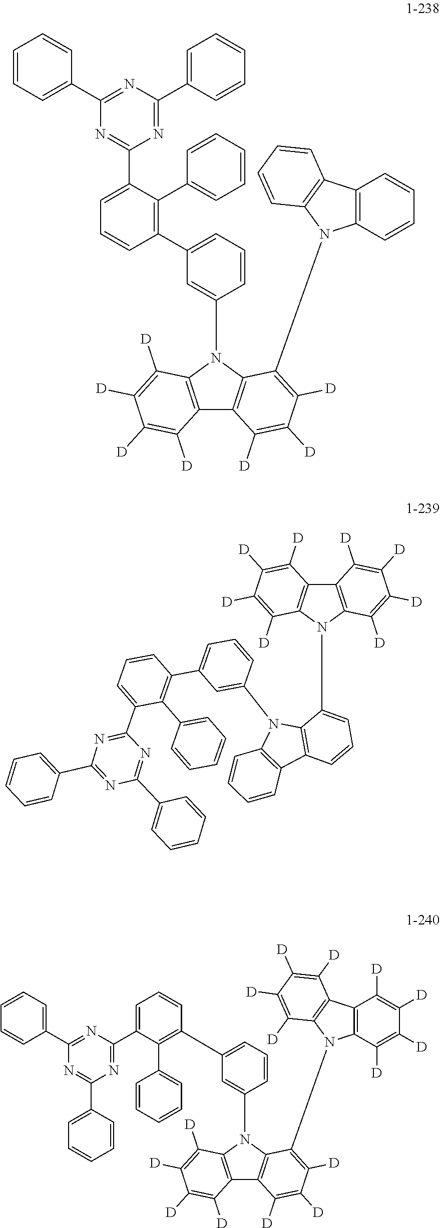 [SMILES: [2H]c1c([2H])c([2H])c2c(c1[2H])c1c([2H])c([2H])c([2H])c(-n3c4ccccc4c4ccccc43)c1n2-c1cccc(-c2cccc(-c3nc(-c4ccccc4)nc(-c4ccccc4)n3)c2-c2ccccc2)c1 ; [2H]c1c([2H])c([2H])c2c(c1[2H])c1c([2H])c([2H])c([2H])c([2H])c1n2-c1cccc2c3ccccc3n(-c3cccc(-c4cccc(-c5nc(-c6ccccc6)nc(-c6ccccc6)n5)c4-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cccc(-c3nc(-c4ccccc4)nc(-c4ccccc4)n3)c2-c2ccccc2)c1]

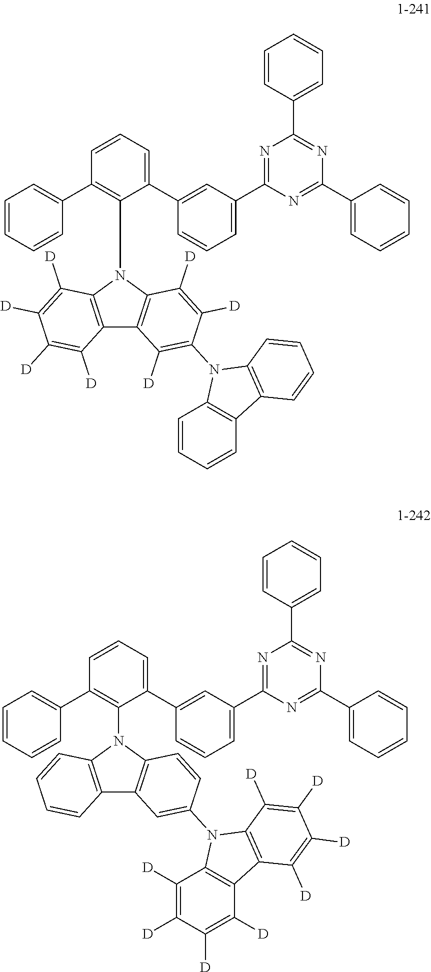 [SMILES: [2H]c1c([2H])c([2H])c2c(c1[2H])c1c([2H])c(-n3c4ccccc4c4ccccc43)c([2H])c([2H])c1n2-c1c(-c2ccccc2)cccc1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1c(-c2ccccc2)cccc1-c1cccc(-c2nc(-c3ccccc3)nc(-c3ccccc3)n2)c1]

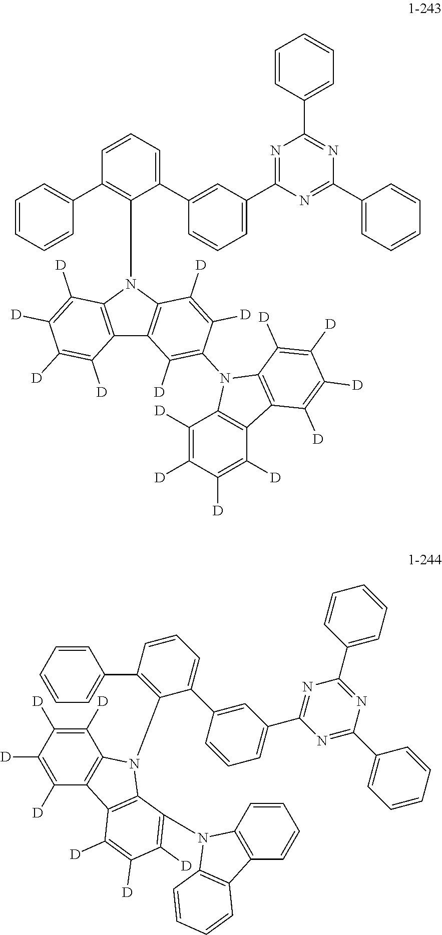 [SMILES: [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1c(-c2ccccc2)cccc1-c1cccc(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1c(-c2ccccc2)cccc1-c1cccc(-c2nc(-c3ccccc3)nc(-c3ccccc3)n2)c1]

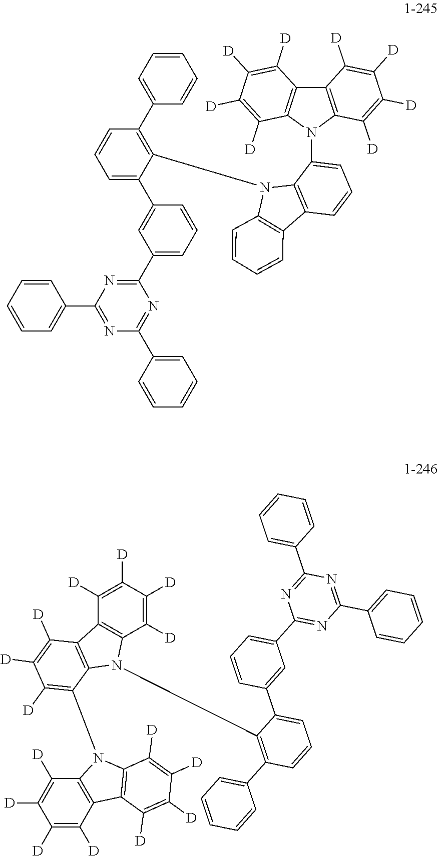 [SMILES: [2H]c1c([2H])c([2H])c2c(c1[2H])c1c([2H])c([2H])c([2H])c([2H])c1n2-c1cccc2c3ccccc3n(-c3c(-c4ccccc4)cccc3-c3cccc(-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1c(-c2ccccc2)cccc1-c1cccc(-c2nc(-c3ccccc3)nc(-c3ccccc3)n2)c1]

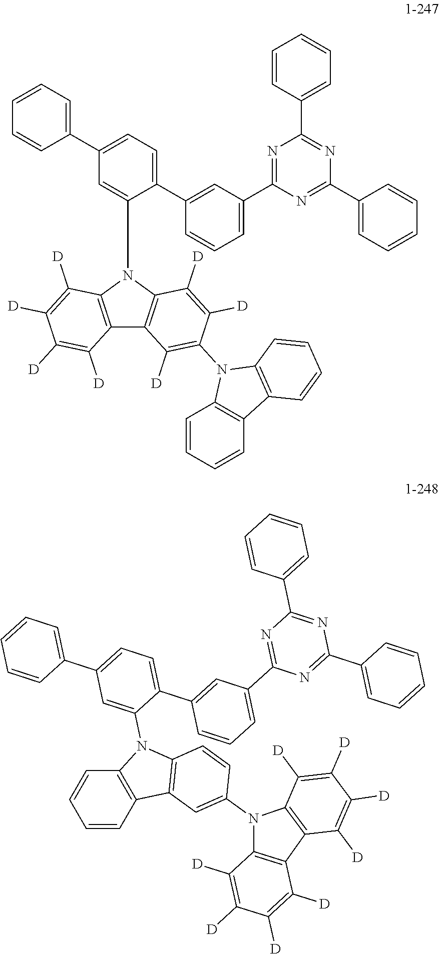 [SMILES: [2H]c1c([2H])c([2H])c2c(c1[2H])c1c([2H])c(-n3c4ccccc4c4ccccc43)c([2H])c([2H])c1n2-c1cc(-c2ccccc2)ccc1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2)ccc1-c1cccc(-c2nc(-c3ccccc3)nc(-c3ccccc3)n2)c1]

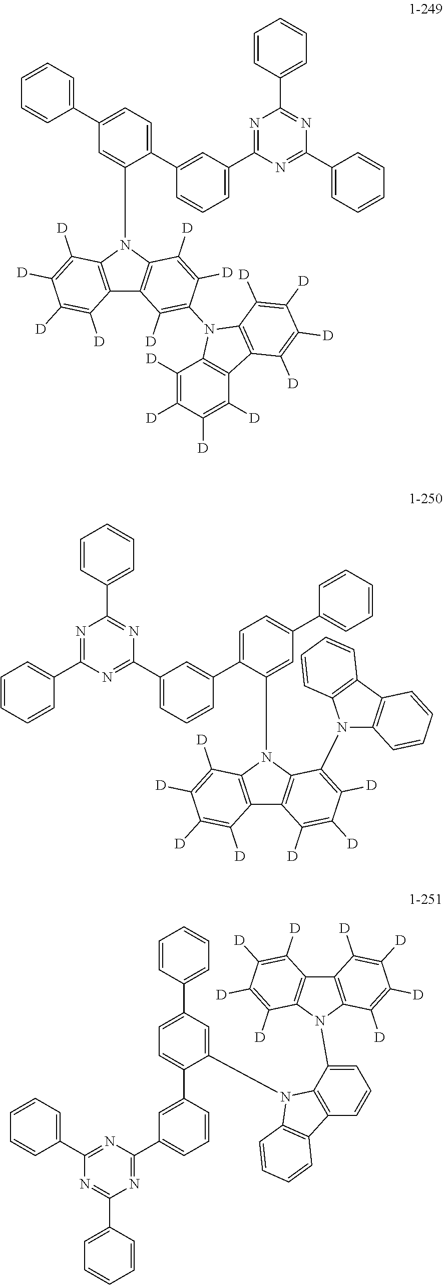

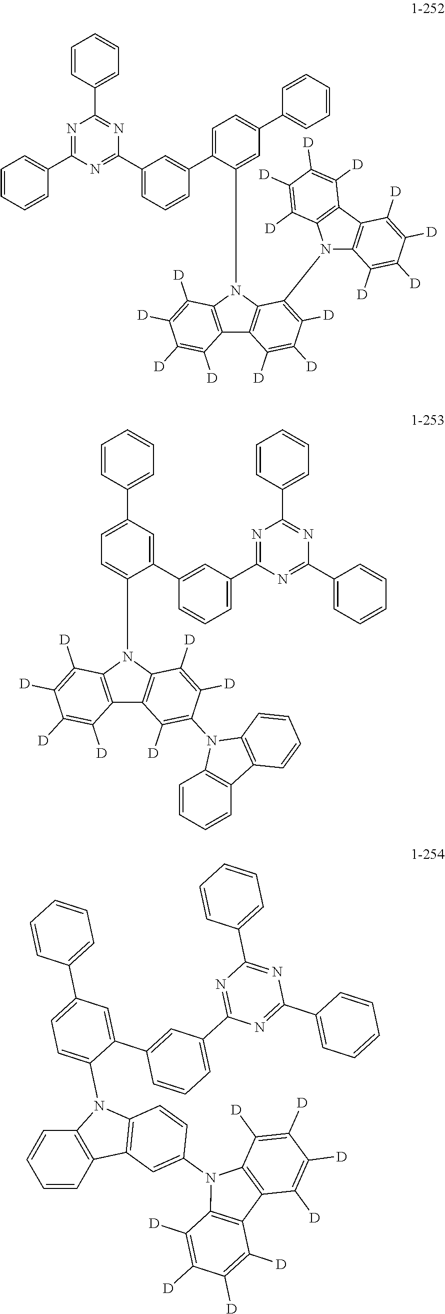

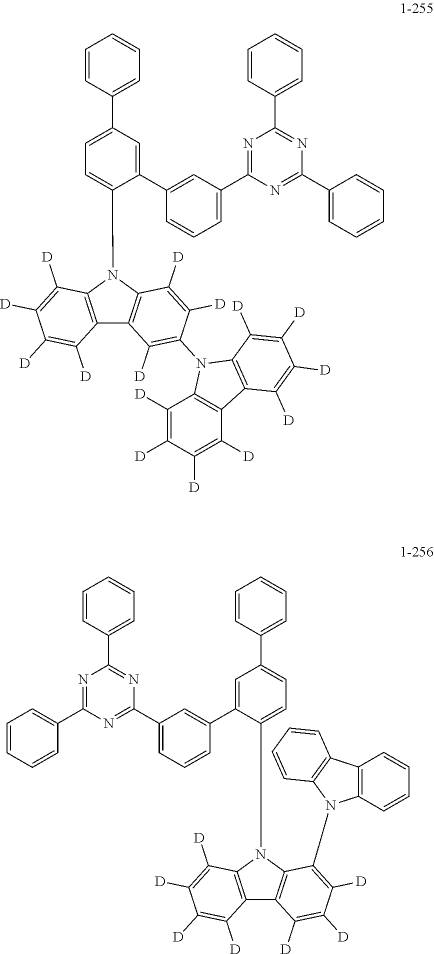 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)cc1-c1cccc(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1ccc(-c2ccccc2)cc1-c1cccc(-c2nc(-c3ccccc3)nc(-c3ccccc3)n2)c1]

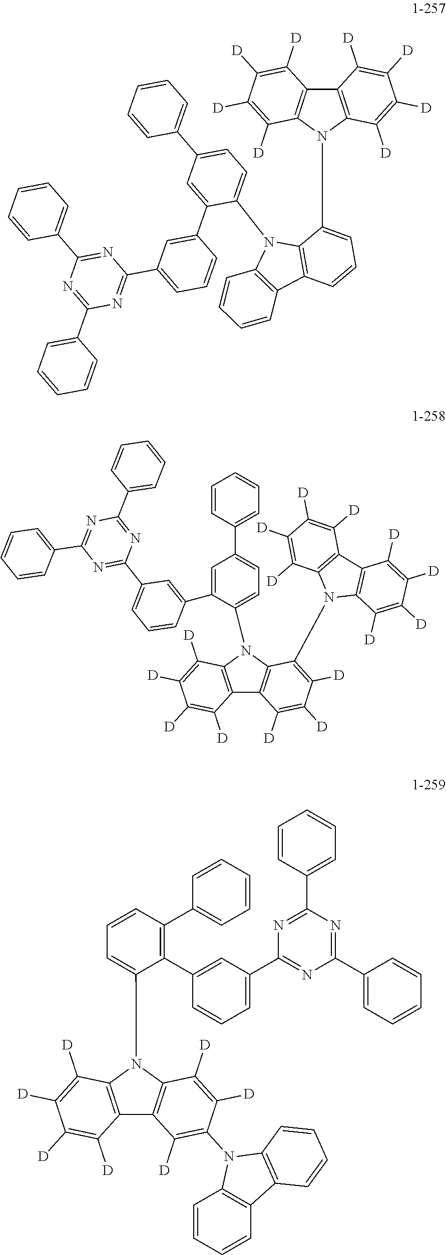 [SMILES: [2H]c1c([2H])c([2H])c2c(c1[2H])c1c([2H])c([2H])c([2H])c([2H])c1n2-c1cccc2c3ccccc3n(-c3ccc(-c4ccccc4)cc3-c3cccc(-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)cc1-c1cccc(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cccc(-c2ccccc2)c1-c1cccc(-c2nc(-c3ccccc3)nc(-c3ccccc3)n2)c1]

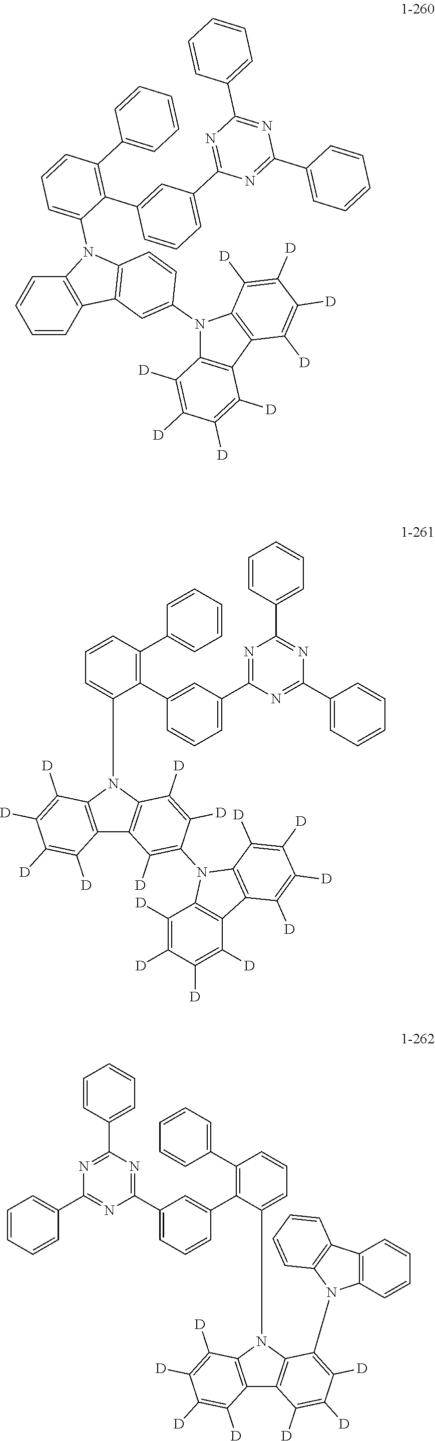 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2ccccc2)c1-c1cccc(-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccccc2)c1-c1cccc(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cccc(-c2ccccc2)c1-c1cccc(-c2nc(-c3ccccc3)nc(-c3ccccc3)n2)c1]

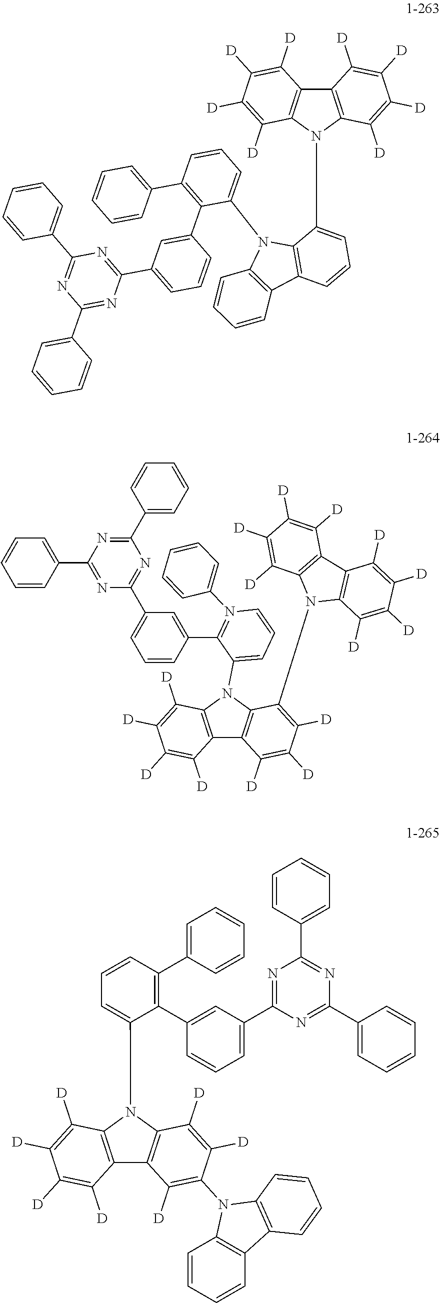

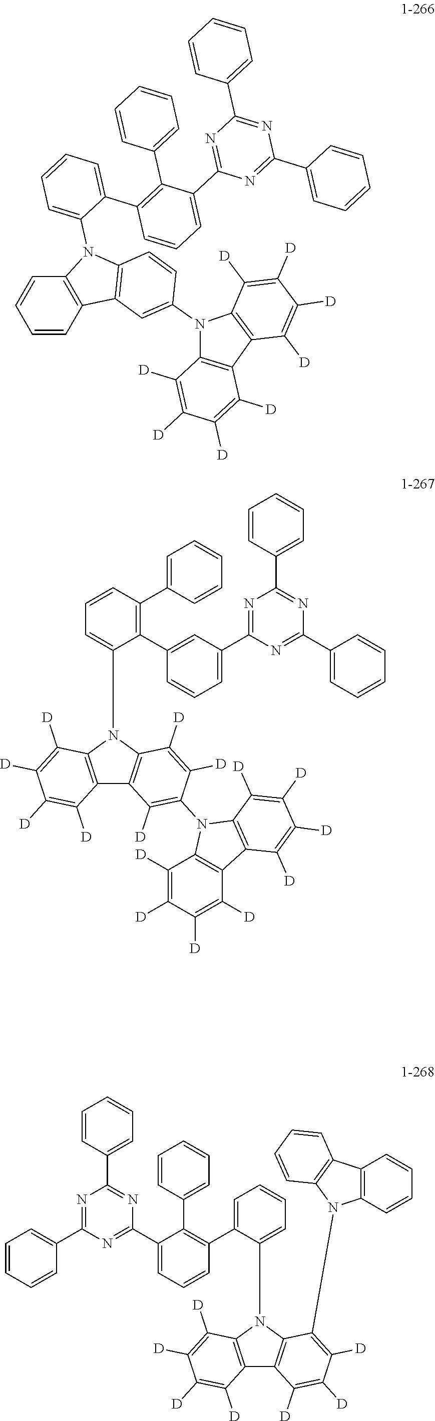 [SMILES: [2H]c1c([2H])c([2H])c2c(c1[2H])c1c([2H])c([2H])c([2H])c([2H])c1n2-c1ccc2c(c1)c1ccccc1n2-c1ccccc1-c1cccc(-c2nc(-c3ccccc3)nc(-c3ccccc3)n2)c1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccccc2)c1-c1cccc(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1ccccc1-c1cccc(-c2nc(-c3ccccc3)nc(-c3ccccc3)n2)c1-c1ccccc1]

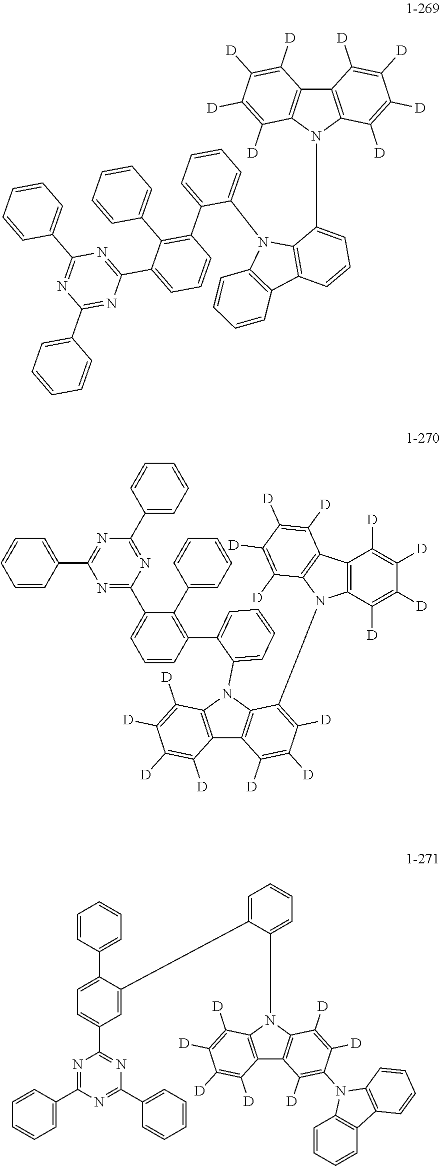 [SMILES: [2H]c1c([2H])c([2H])c2c(c1[2H])c1c([2H])c([2H])c([2H])c([2H])c1n2-c1cccc2c3ccccc3n(-c3ccccc3-c3cccc(-c4nc(-c5ccccc5)nc(-c5ccccc5)n4)c3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1cccc(-c2nc(-c3ccccc3)nc(-c3ccccc3)n2)c1-c1ccccc1 ; [2H]c1c([2H])c([2H])c2c(c1[2H])c1c([2H])c(-n3c4ccccc4c4ccccc43)c([2H])c([2H])c1n2-c1ccccc1-c1cc(-c2nc(-c3ccccc3)nc(-c3ccccc3)n2)ccc1-c1ccccc1]

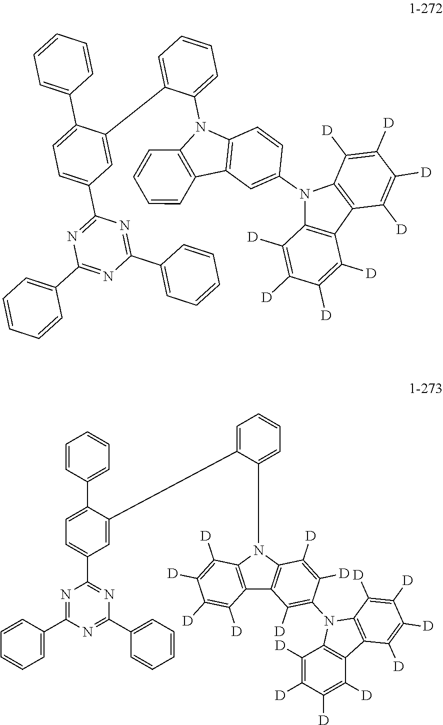 [SMILES: [2H]c1c([2H])c([2H])c2c(c1[2H])c1c([2H])c([2H])c([2H])c([2H])c1n2-c1ccc2c(c1)c1ccccc1n2-c1ccccc1-c1cc(-c2nc(-c3ccccc3)nc(-c3ccccc3)n2)ccc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1cc(-c2nc(-c3ccccc3)nc(-c3ccccc3)n2)ccc1-c1ccccc1]

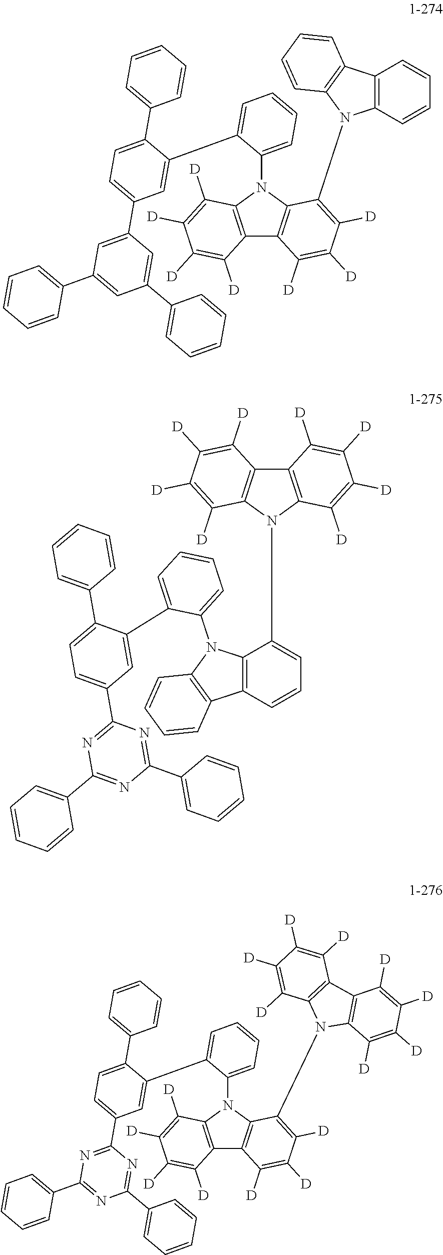 [SMILES: [2H]c1c([2H])c([2H])c2c(c1[2H])c1c([2H])c([2H])c([2H])c(-n3c4ccccc4c4ccccc43)c1n2-c1ccccc1-c1cc(-c2cc(-c3ccccc3)cc(-c3ccccc3)c2)ccc1-c1ccccc1 ; [2H]c1c([2H])c([2H])c2c(c1[2H])c1c([2H])c([2H])c([2H])c([2H])c1n2-c1cccc2c3ccccc3n(-c3ccccc3-c3cc(-c4nc(-c5ccccc5)nc(-c5ccccc5)n4)ccc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1cc(-c2nc(-c3ccccc3)nc(-c3ccccc3)n2)ccc1-c1ccccc1]

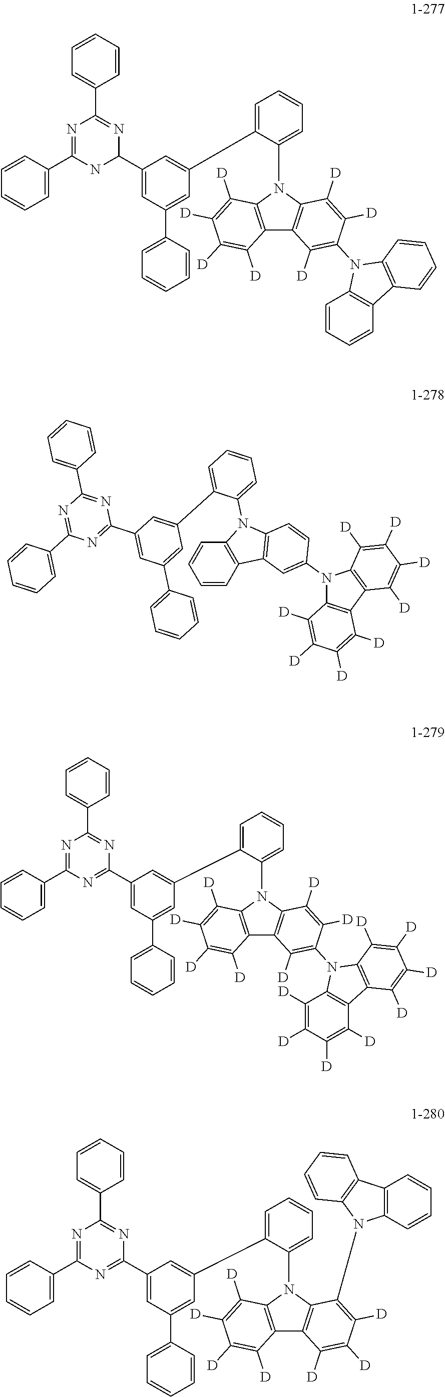

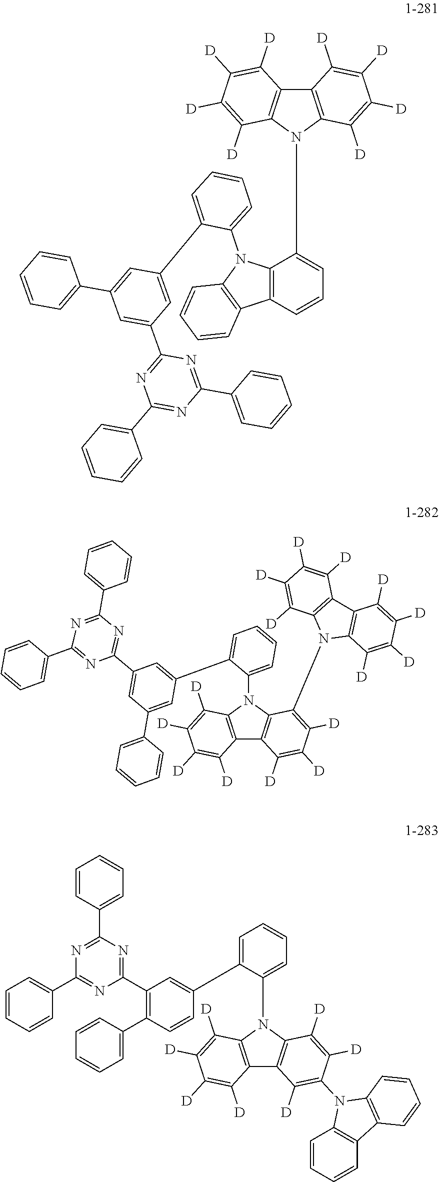 [SMILES: [2H]c1c([2H])c([2H])c2c(c1[2H])c1c([2H])c([2H])c([2H])c([2H])c1n2-c1cccc2c3ccccc3n(-c3ccccc3-c3cc(-c4ccccc4)cc(-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1cc(-c2ccccc2)cc(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1ccccc1-c1ccc(-c2ccccc2)c(-c2nc(-c3ccccc3)nc(-c3ccccc3)n2)c1]

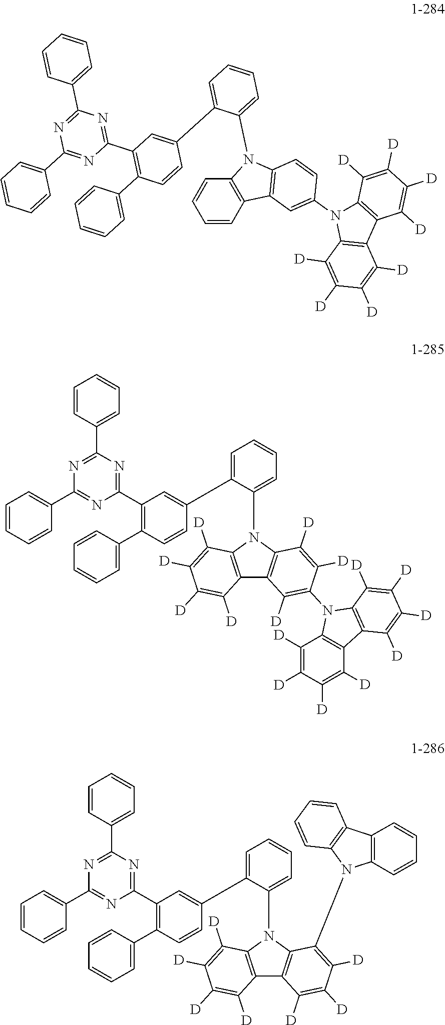 [SMILES: [2H]c1c([2H])c([2H])c2c(c1[2H])c1c([2H])c([2H])c([2H])c([2H])c1n2-c1ccc2c(c1)c1ccccc1n2-c1ccccc1-c1ccc(-c2ccccc2)c(-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1ccc(-c2ccccc2)c(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1ccccc1-c1ccc(-c2ccccc2)c(-c2nc(-c3ccccc3)nc(-c3ccccc3)n2)c1]

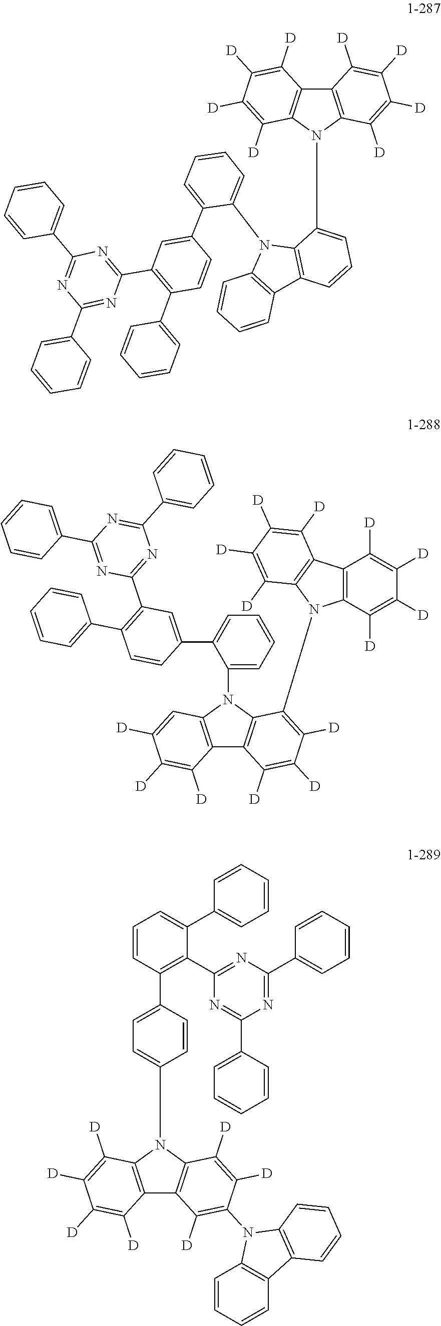 [SMILES: [2H]c1c([2H])c([2H])c2c(c1[2H])c1c([2H])c([2H])c([2H])c([2H])c1n2-c1cccc2c3ccccc3n(-c3ccccc3-c3ccc(-c4ccccc4)c(-c4nc(-c5ccccc5)nc(-c5ccccc5)n4)c3)c12 ; [2H]c1cc2c(c([2H])c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1ccc(-c2ccccc2)c(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1ccc(-c2cccc(-c3ccccc3)c2-c2nc(-c3ccccc3)nc(-c3ccccc3)n2)cc1]

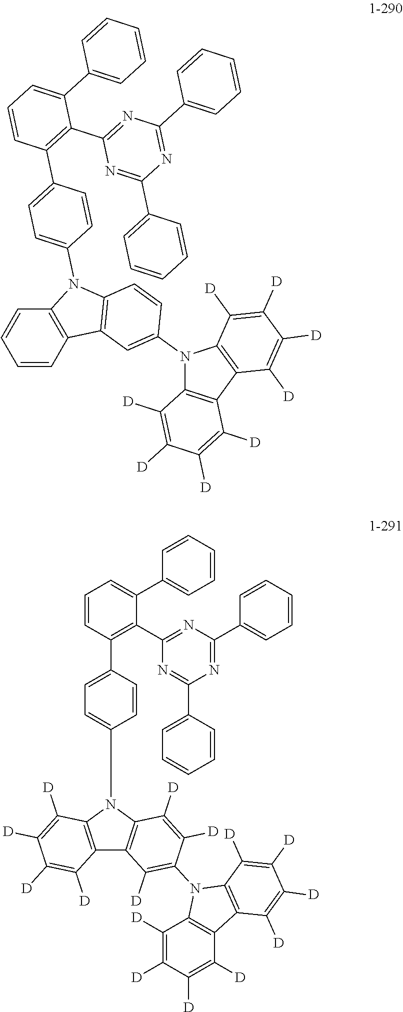 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2cccc(-c3ccccc3)c2-c2nc(-c3ccccc3)nc(-c3ccccc3)n2)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cccc(-c3ccccc3)c2-c2nc(-c3ccccc3)nc(-c3ccccc3)n2)cc1]

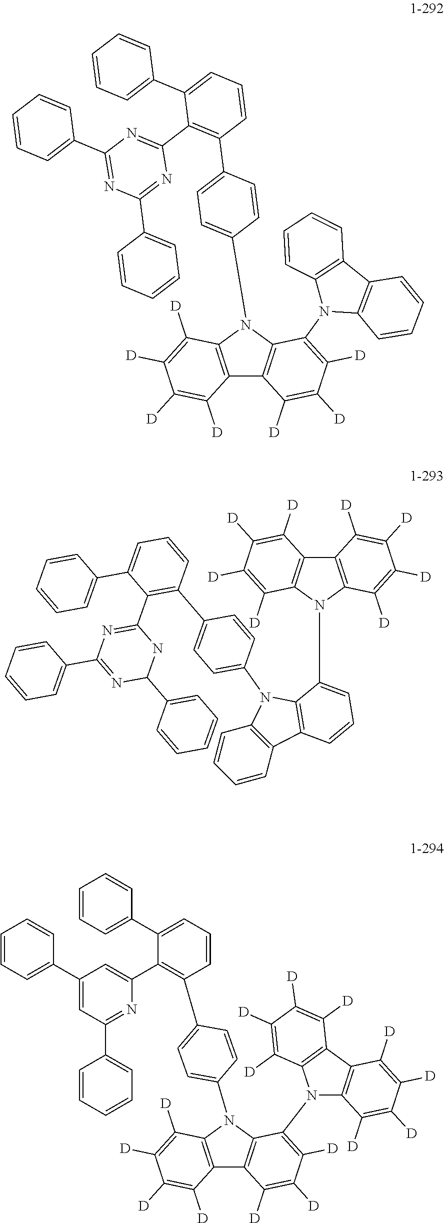

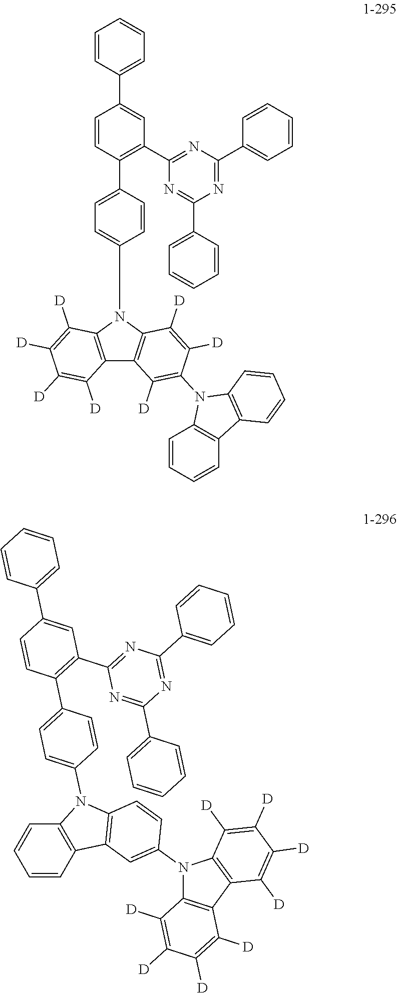 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3ccccc3)cc2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3ccccc3)cc2-c2nc(-c3ccccc3)nc(-c3ccccc3)n2)cc1]

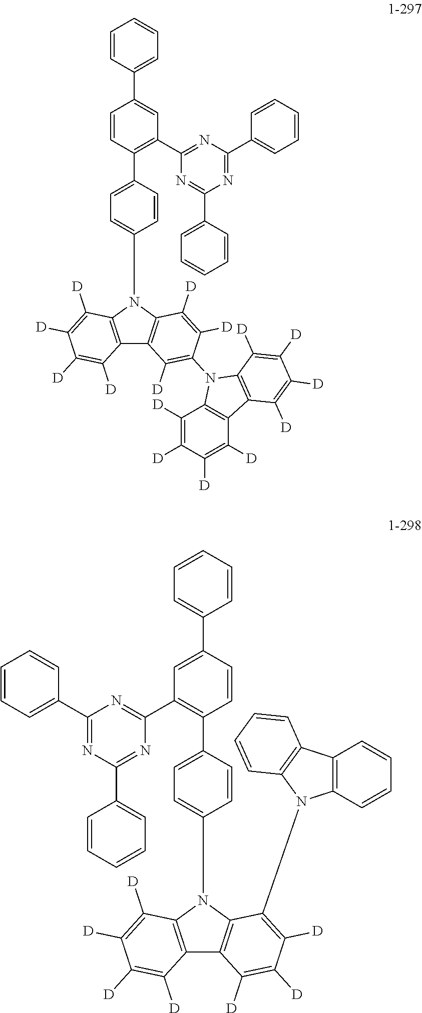 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3ccccc3)cc2-c2nc(-c3ccccc3)nc(-c3ccccc3)n2)cc1 ; [2H]c1c([2H])c([2H])c2c(c1[2H])c1c([2H])c([2H])c([2H])c(-n3c4ccccc4c4ccccc43)c1n2-c1ccc(-c2ccc(-c3ccccc3)cc2-c2nc(-c3ccccc3)nc(-c3ccccc3)n2)cc1]

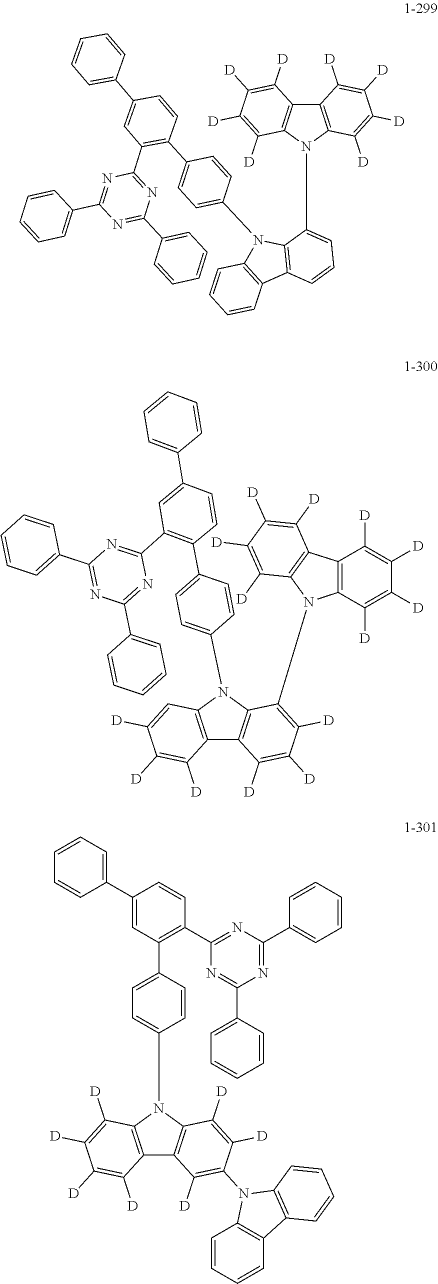 [SMILES: [2H]c1c([2H])c([2H])c2c(c1[2H])c1c([2H])c([2H])c([2H])c([2H])c1n2-c1cccc2c3ccccc3n(-c3ccc(-c4ccc(-c5ccccc5)cc4-c4nc(-c5ccccc5)nc(-c5ccccc5)n4)cc3)c12 ; [2H]c1cc2c(c([2H])c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3ccccc3)cc2-c2nc(-c3ccccc3)nc(-c3ccccc3)n2)cc1 ; [2H]c1c([2H])c([2H])c2c(c1[2H])c1c([2H])c(-n3c4ccccc4c4ccccc43)c([2H])c([2H])c1n2-c1ccc(-c2cc(-c3ccccc3)ccc2-c2nc(-c3ccccc3)nc(-c3ccccc3)n2)cc1]

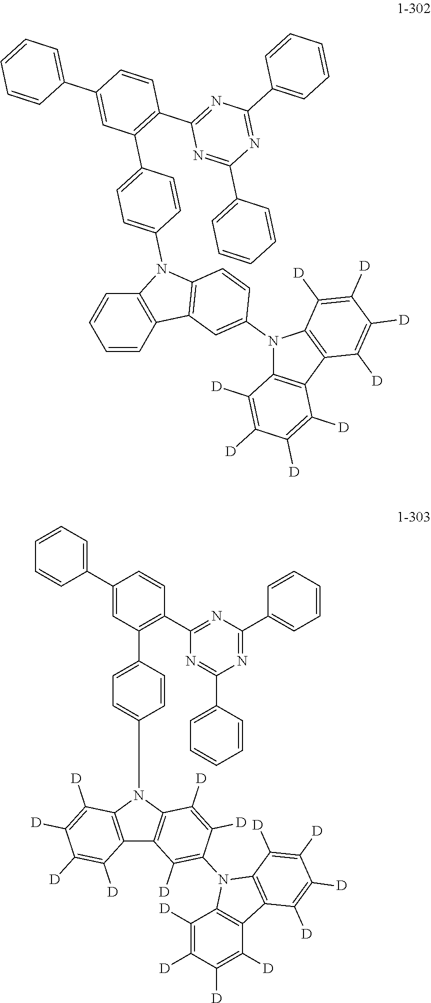 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2cc(-c3ccccc3)ccc2-c2nc(-c3ccccc3)nc(-c3ccccc3)n2)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cc(-c3ccccc3)ccc2-c2nc(-c3ccccc3)nc(-c3ccccc3)n2)cc1]

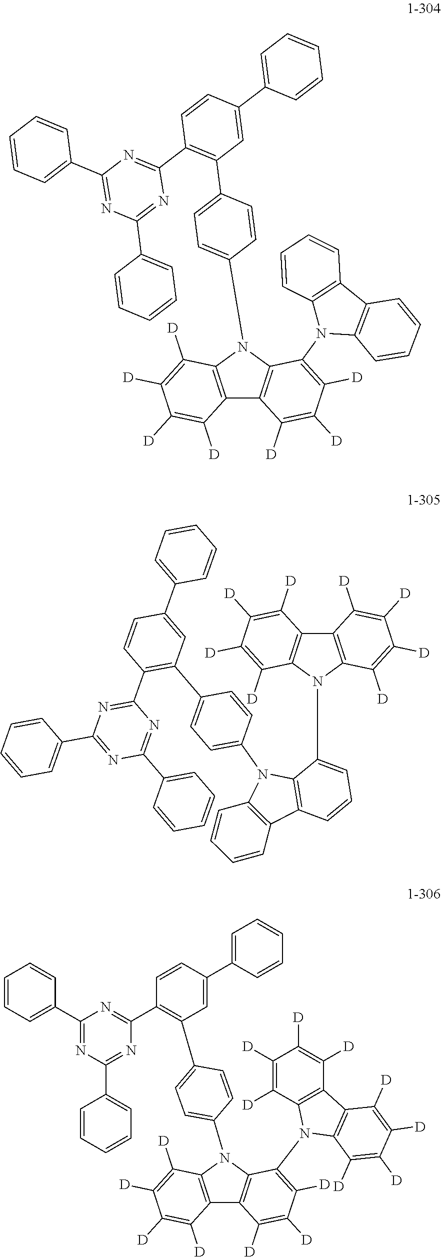 [SMILES: [2H]c1c([2H])c([2H])c2c(c1[2H])c1c([2H])c([2H])c([2H])c(-n3c4ccccc4c4ccccc43)c1n2-c1ccc(-c2cc(-c3ccccc3)ccc2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1cccc2c3ccccc3n(-c3ccc(-c4cc(-c5ccccc5)ccc4-c4nc(-c5ccccc5)nc(-c5ccccc5)n4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2cc(-c3ccccc3)ccc2-c2nc(-c3ccccc3)nc(-c3ccccc3)n2)cc1]

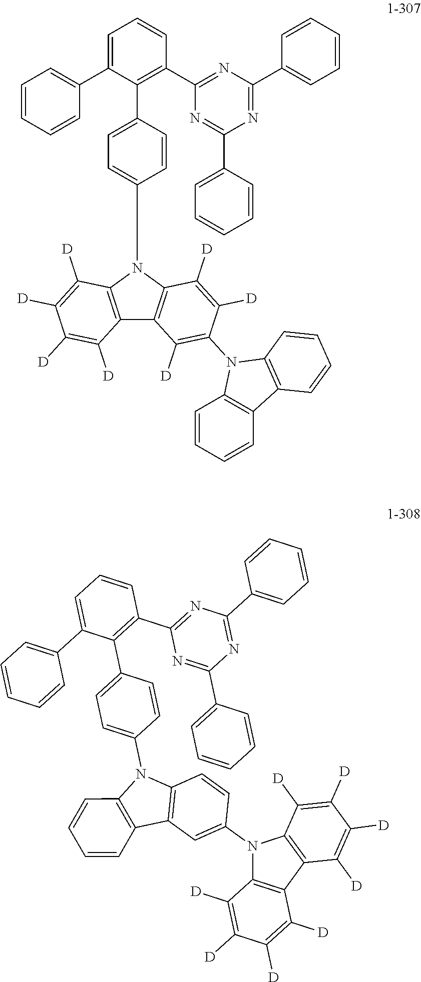 [SMILES: [2H]c1c([2H])c([2H])c2c(c1[2H])c1c([2H])c(-n3c4ccccc4c4ccccc43)c([2H])c([2H])c1n2-c1ccc(-c2c(-c3ccccc3)cccc2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2c(-c3ccccc3)cccc2-c2nc(-c3ccccc3)nc(-c3ccccc3)n2)cc1]

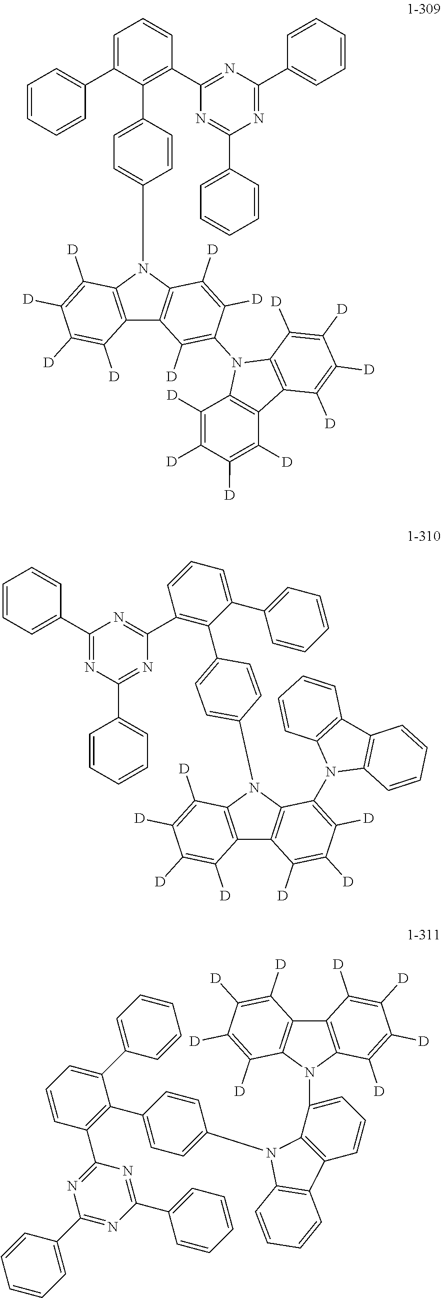 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2c(-c3ccccc3)cccc2-c2nc(-c3ccccc3)nc(-c3ccccc3)n2)cc1 ; [2H]c1c([2H])c([2H])c2c(c1[2H])c1c([2H])c([2H])c([2H])c(-n3c4ccccc4c4ccccc43)c1n2-c1ccc(-c2c(-c3ccccc3)cccc2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1cccc2c3ccccc3n(-c3ccc(-c4c(-c5ccccc5)cccc4-c4nc(-c5ccccc5)nc(-c5ccccc5)n4)cc3)c12]

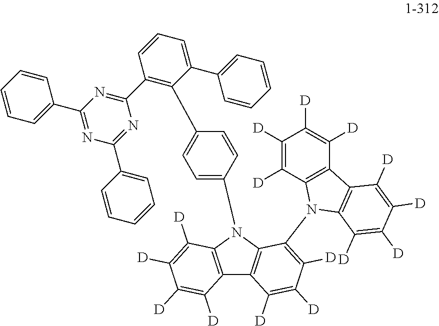 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2c(-c3ccccc3)cccc2-c2nc(-c3ccccc3)nc(-c3ccccc3)n2)cc1]

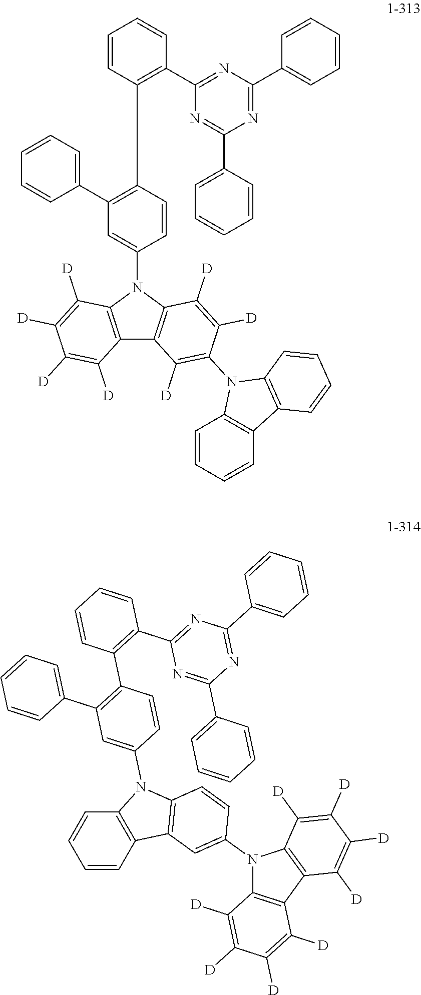 [SMILES: [2H]c1c([2H])c([2H])c2c(c1[2H])c1c([2H])c(-n3c4ccccc4c4ccccc43)c([2H])c([2H])c1n2-c1ccc(-c2ccccc2-c2nc(-c3ccccc3)nc(-c3ccccc3)n2)c(-c2ccccc2)c1 ; [2H]c1c([2H])c([2H])c2c(c1[2H])c1c([2H])c([2H])c([2H])c([2H])c1n2-c1ccc2c(c1)c1ccccc1n2-c1ccc(-c2ccccc2-c2nc(-c3ccccc3)nc(-c3ccccc3)n2)c(-c2ccccc2)c1]

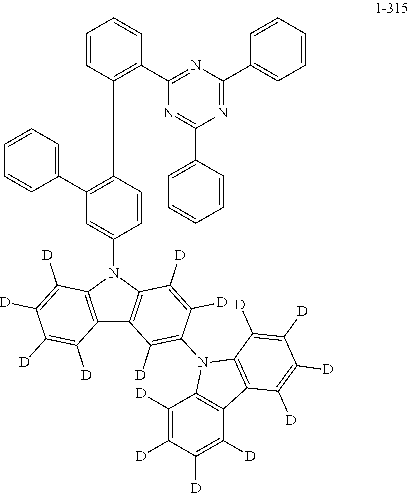 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2-c2nc(-c3ccccc3)nc(-c3ccccc3)n2)c(-c2ccccc2)c1]

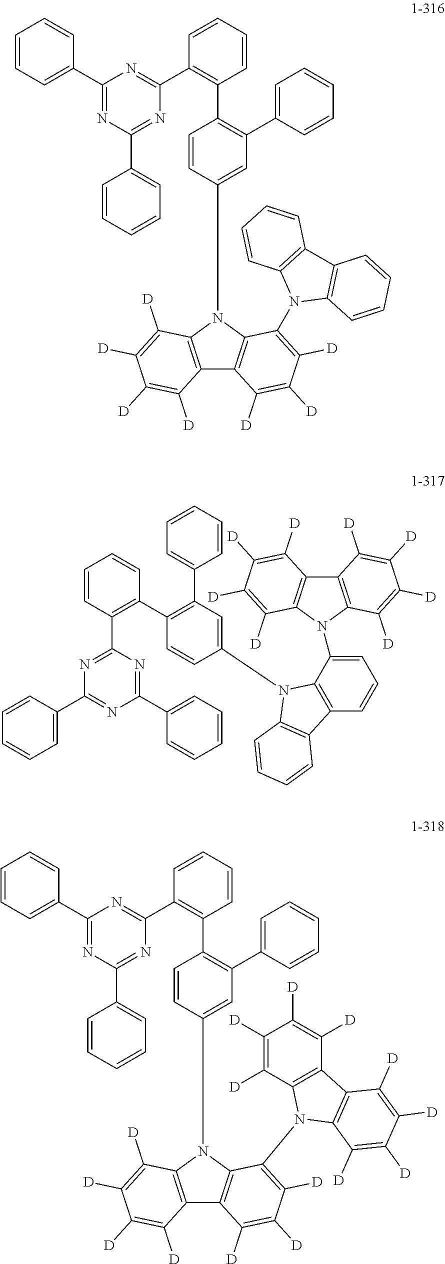 [SMILES: [2H]c1c([2H])c([2H])c2c(c1[2H])c1c([2H])c([2H])c([2H])c(-n3c4ccccc4c4ccccc43)c1n2-c1ccc(-c2ccccc2-c2nc(-c3ccccc3)nc(-c3ccccc3)n2)c(-c2ccccc2)c1 ; [2H]c1c([2H])c([2H])c2c(c1[2H])c1c([2H])c([2H])c([2H])c([2H])c1n2-c1cccc2c3ccccc3n(-c3ccc(-c4ccccc4-c4nc(-c5ccccc5)nc(-c5ccccc5)n4)c(-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2-c2nc(-c3ccccc3)nc(-c3ccccc3)n2)c(-c2ccccc2)c1]

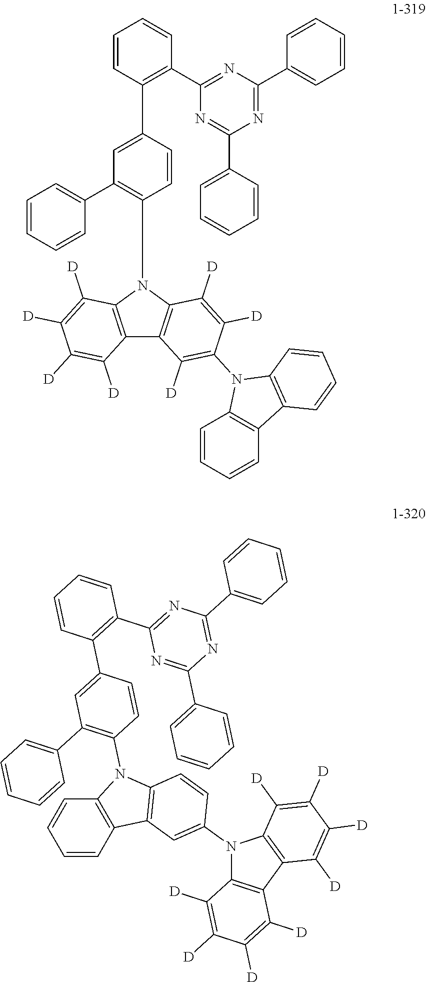 [SMILES: [2H]c1c([2H])c([2H])c2c(c1[2H])c1c([2H])c(-n3c4ccccc4c4ccccc43)c([2H])c([2H])c1n2-c1ccc(-c2ccccc2-c2nc(-c3ccccc3)nc(-c3ccccc3)n2)cc1-c1ccccc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccccc2-c2nc(-c3ccccc3)nc(-c3ccccc3)n2)cc1-c1ccccc1]

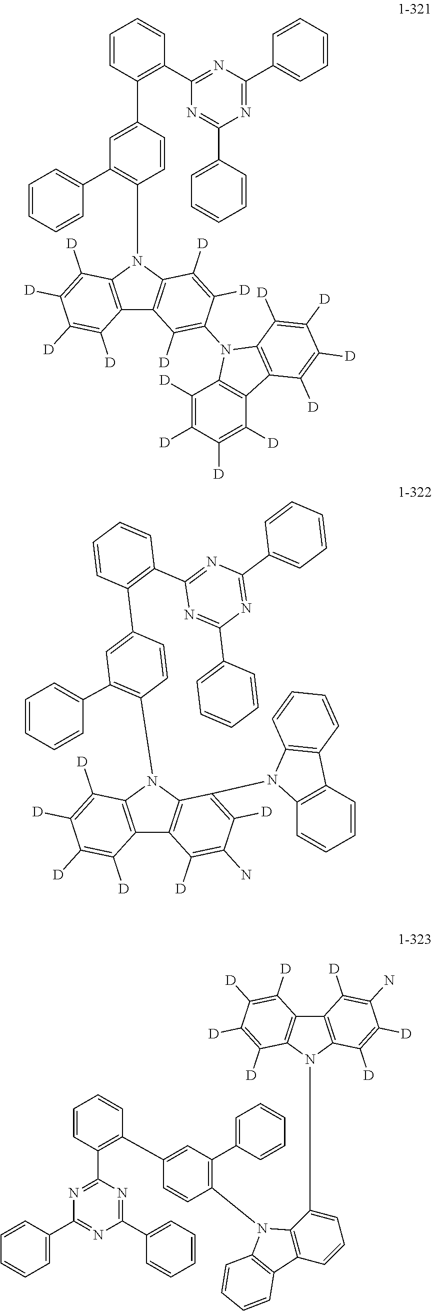 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2-c2nc(-c3ccccc3)nc(-c3ccccc3)n2)cc1-c1ccccc1 ; [2H]c1c([2H])c([2H])c2c(c1[2H])c1c([2H])c(N)c([2H])c(-n3c4ccccc4c4ccccc43)c1n2-c1ccc(-c2ccccc2-c2nc(-c3ccccc3)nc(-c3ccccc3)n2)cc1-c1ccccc1 ; [2H]c1c([2H])c([2H])c2c(c1[2H])c1c([2H])c(N)c([2H])c([2H])c1n2-c1cccc2c3ccccc3n(-c3ccc(-c4ccccc4-c4nc(-c5ccccc5)nc(-c5ccccc5)n4)cc3-c3ccccc3)c12]

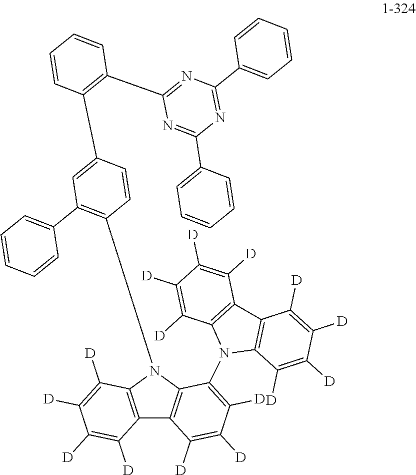 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2-c2nc(-c3ccccc3)nc(-c3ccccc3)n2)cc1-c1ccccc1]

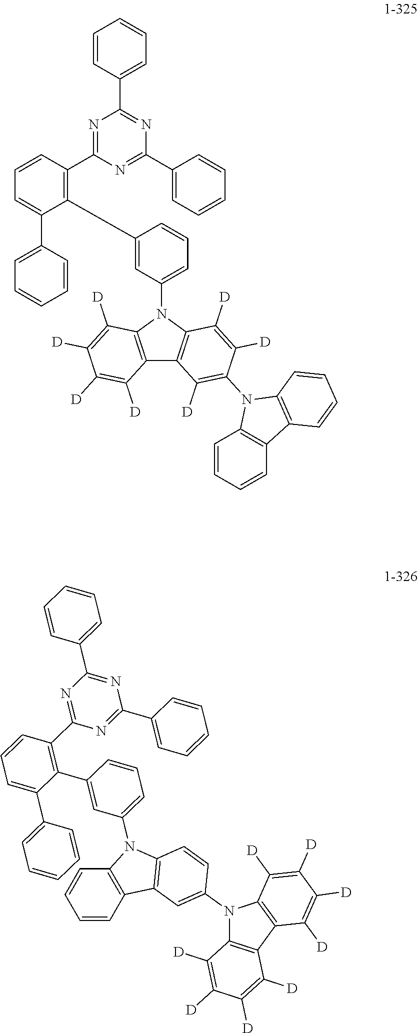 [SMILES: [2H]c1c([2H])c([2H])c2c(c1[2H])c1c([2H])c(-n3c4ccccc4c4ccccc43)c([2H])c([2H])c1n2-c1cccc(-c2c(-c3ccccc3)cccc2-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1cccc(-c2c(-c3ccccc3)cccc2-c2nc(-c3ccccc3)nc(-c3ccccc3)n2)c1]

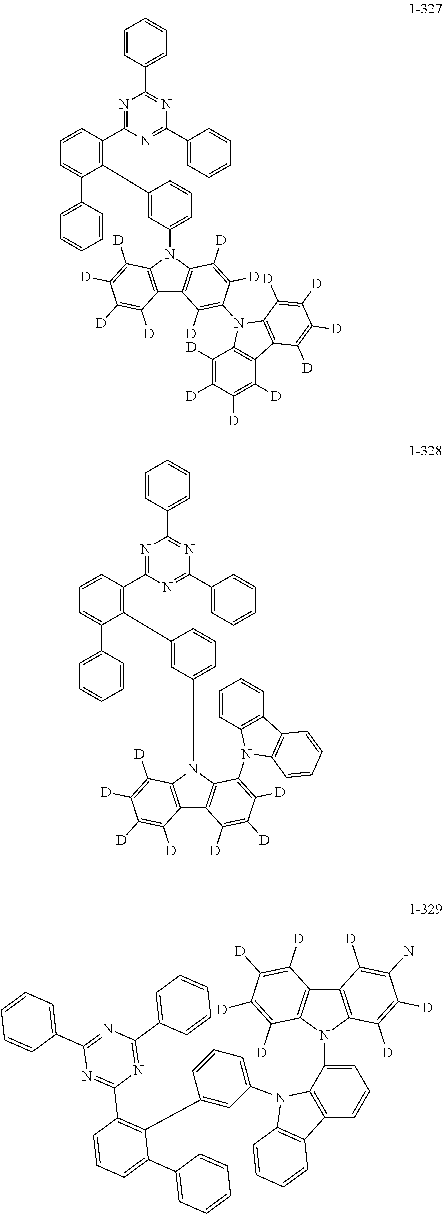 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2c(-c3ccccc3)cccc2-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cccc(-c2c(-c3ccccc3)cccc2-c2nc(-c3ccccc3)nc(-c3ccccc3)n2)c1 ; [2H]c1c([2H])c([2H])c2c(c1[2H])c1c([2H])c(N)c([2H])c([2H])c1n2-c1cccc2c3ccccc3n(-c3cccc(-c4c(-c5ccccc5)cccc4-c4nc(-c5ccccc5)nc(-c5ccccc5)n4)c3)c12]

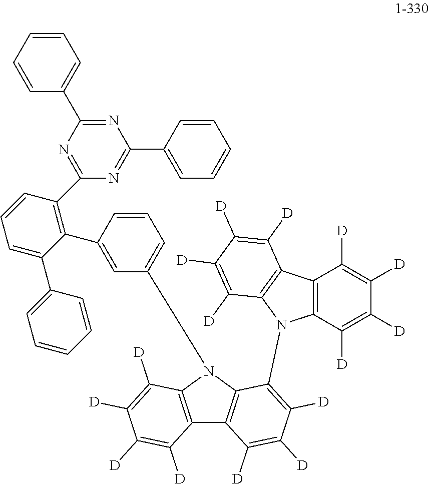 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2c(-c3ccccc3)cccc2-c2nc(-c3ccccc3)nc(-c3ccccc3)n2)c1]

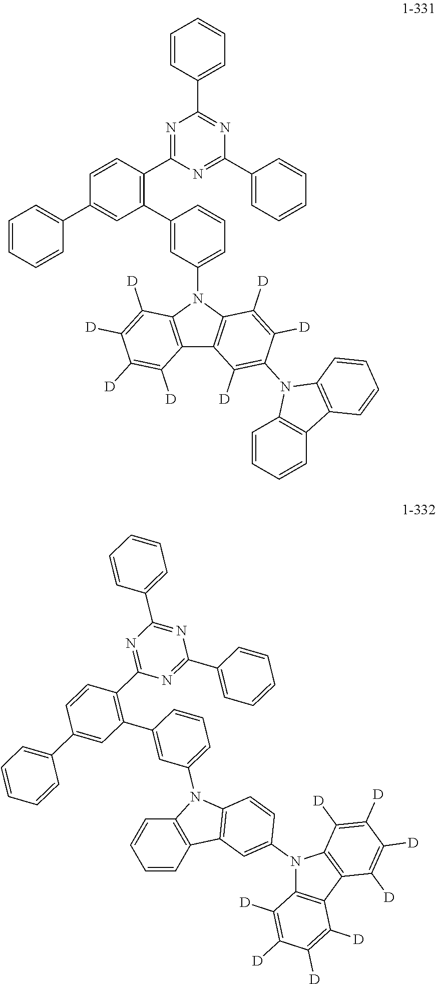 [SMILES: [2H]c1c([2H])c([2H])c2c(c1[2H])c1c([2H])c(-n3c4ccccc4c4ccccc43)c([2H])c([2H])c1n2-c1cccc(-c2cc(-c3ccccc3)ccc2-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1cccc(-c2cc(-c3ccccc3)ccc2-c2nc(-c3ccccc3)nc(-c3ccccc3)n2)c1]

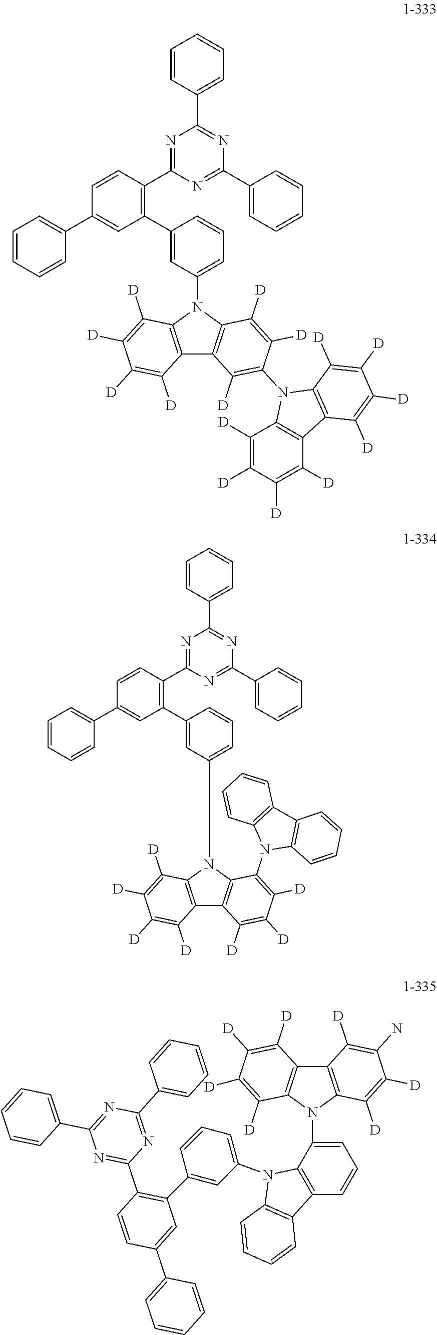 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cc(-c3ccccc3)ccc2-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cccc(-c2cc(-c3ccccc3)ccc2-c2nc(-c3ccccc3)nc(-c3ccccc3)n2)c1 ; [2H]c1c([2H])c([2H])c2c(c1[2H])c1c([2H])c(N)c([2H])c([2H])c1n2-c1cccc2c3ccccc3n(-c3cccc(-c4cc(-c5ccccc5)ccc4-c4nc(-c5ccccc5)nc(-c5ccccc5)n4)c3)c12]

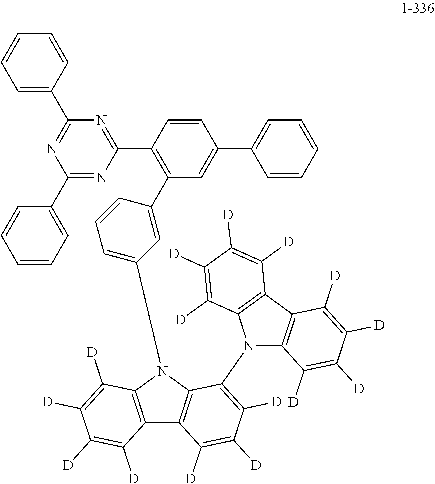 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cc(-c3ccccc3)ccc2-c2nc(-c3ccccc3)nc(-c3ccccc3)n2)c1]

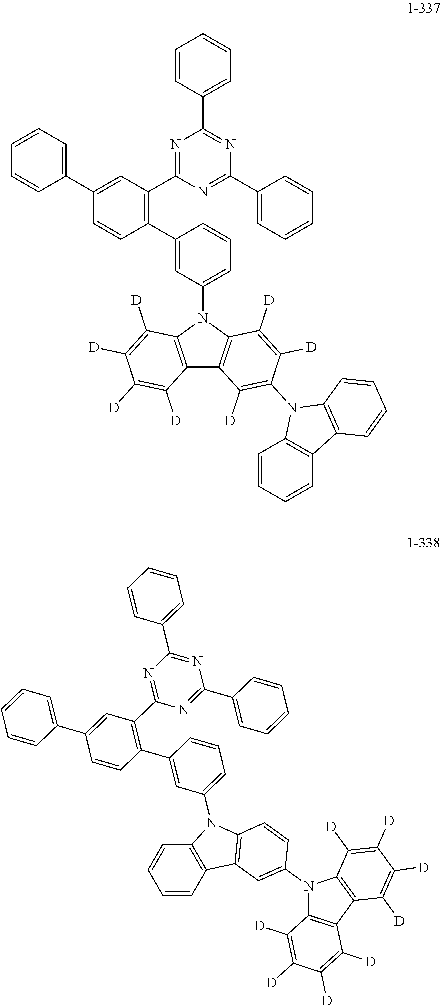 [SMILES: [2H]c1c([2H])c([2H])c2c(c1[2H])c1c([2H])c(-n3c4ccccc4c4ccccc43)c([2H])c([2H])c1n2-c1cccc(-c2ccc(-c3ccccc3)cc2-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1cccc(-c2ccc(-c3ccccc3)cc2-c2nc(-c3ccccc3)nc(-c3ccccc3)n2)c1]

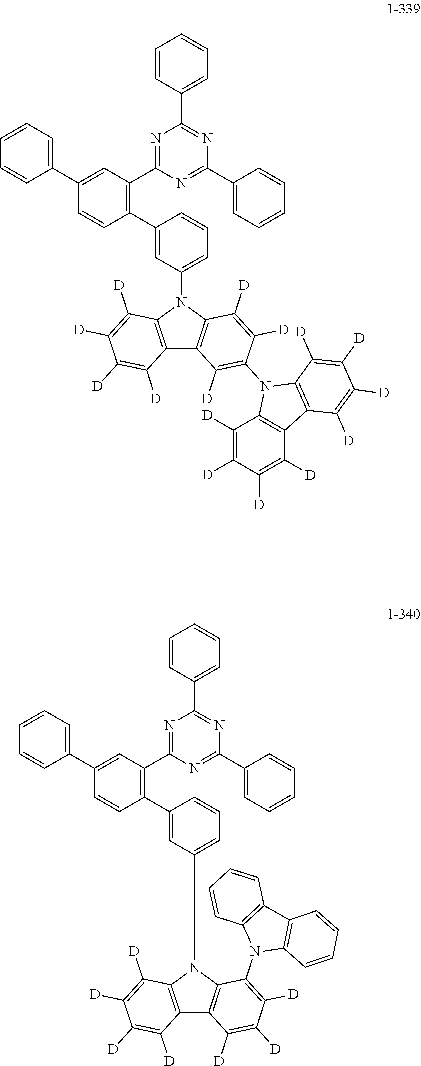 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccc(-c3ccccc3)cc2-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cccc(-c2ccc(-c3ccccc3)cc2-c2nc(-c3ccccc3)nc(-c3ccccc3)n2)c1]

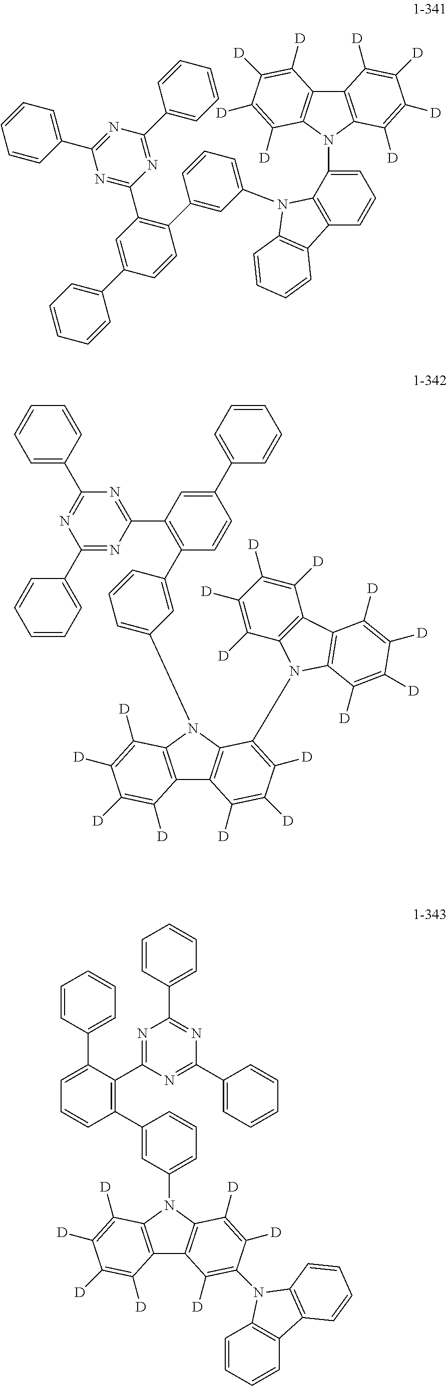 [SMILES: [2H]c1c([2H])c([2H])c2c(c1[2H])c1c([2H])c([2H])c([2H])c([2H])c1n2-c1cccc2c3ccccc3n(-c3cccc(-c4ccc(-c5ccccc5)cc4-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccc(-c3ccccc3)cc2-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cccc(-c2cccc(-c3ccccc3)c2-c2nc(-c3ccccc3)nc(-c3ccccc3)n2)c1]

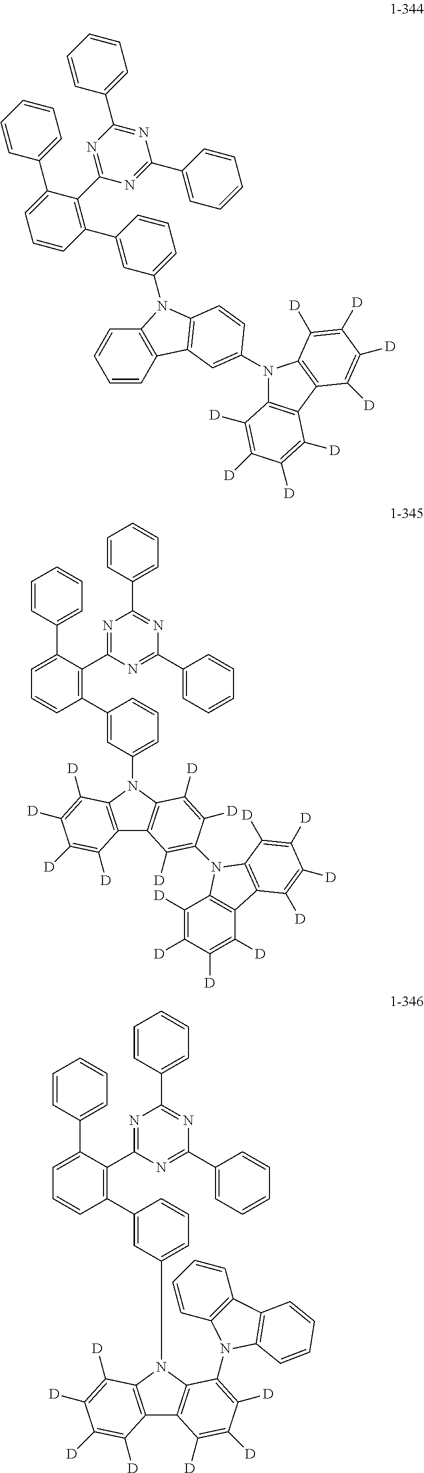 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2cccc(-c3ccccc3)c2-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cccc(-c3ccccc3)c2-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cccc(-c2cccc(-c3ccccc3)c2-c2nc(-c3ccccc3)nc(-c3ccccc3)n2)c1]

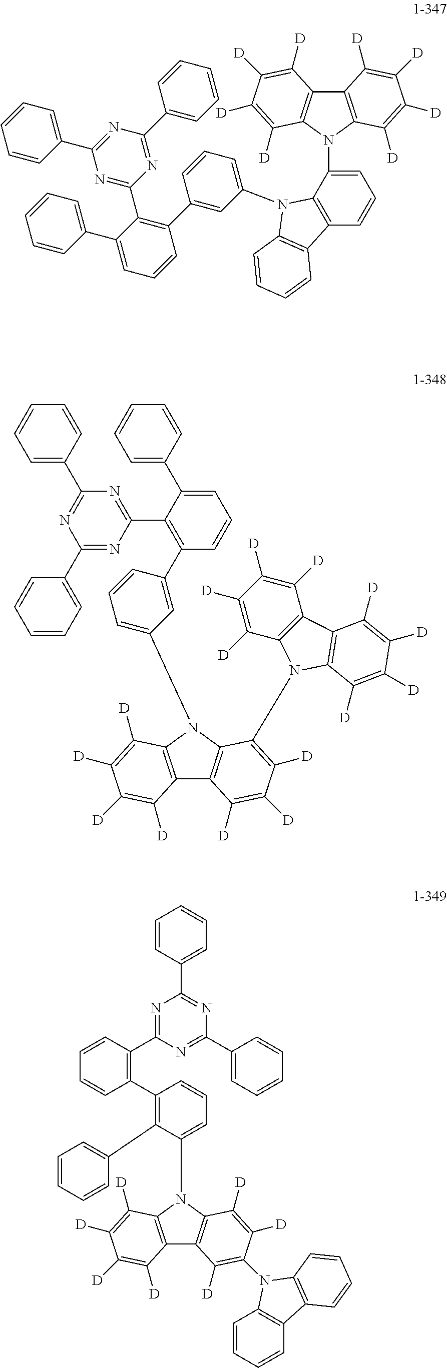 [SMILES: [2H]c1c([2H])c([2H])c2c(c1[2H])c1c([2H])c([2H])c([2H])c([2H])c1n2-c1cccc2c3ccccc3n(-c3cccc(-c4cccc(-c5ccccc5)c4-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cccc(-c3ccccc3)c2-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cccc(-c2ccccc2-c2nc(-c3ccccc3)nc(-c3ccccc3)n2)c1-c1ccccc1]

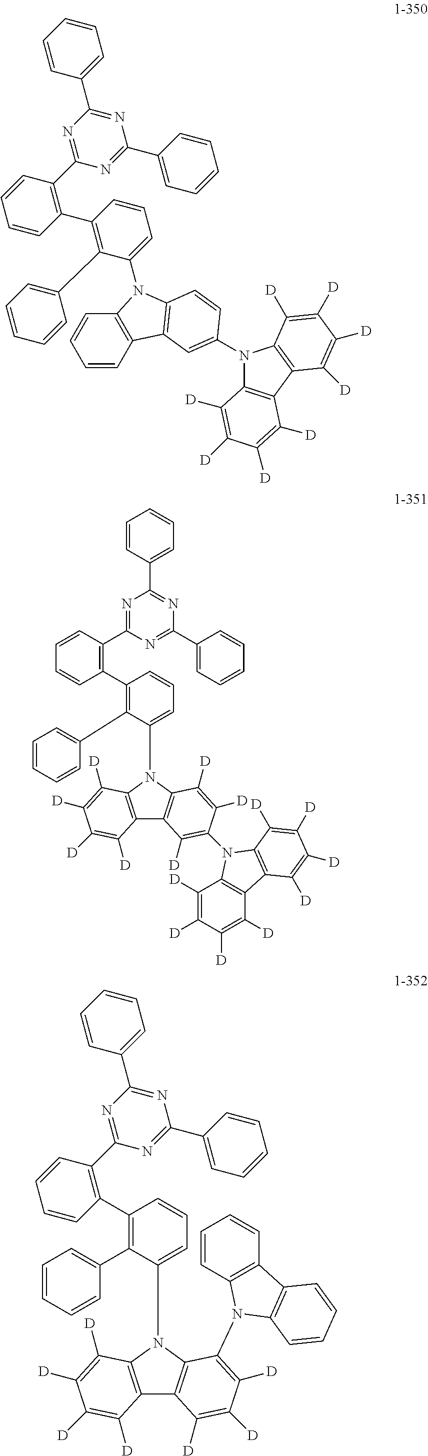 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2ccccc2-c2nc(-c3ccccc3)nc(-c3ccccc3)n2)c1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccccc2-c2nc(-c3ccccc3)nc(-c3ccccc3)n2)c1-c1ccccc1 ; [2H]c1c([2H])c([2H])c2c(c1[2H])c1c([2H])c([2H])c([2H])c(-n3c4ccccc4c4ccccc43)c1n2-c1cccc(-c2ccccc2-c2nc(-c3ccccc3)nc(-c3ccccc3)n2)c1-c1ccccc1]

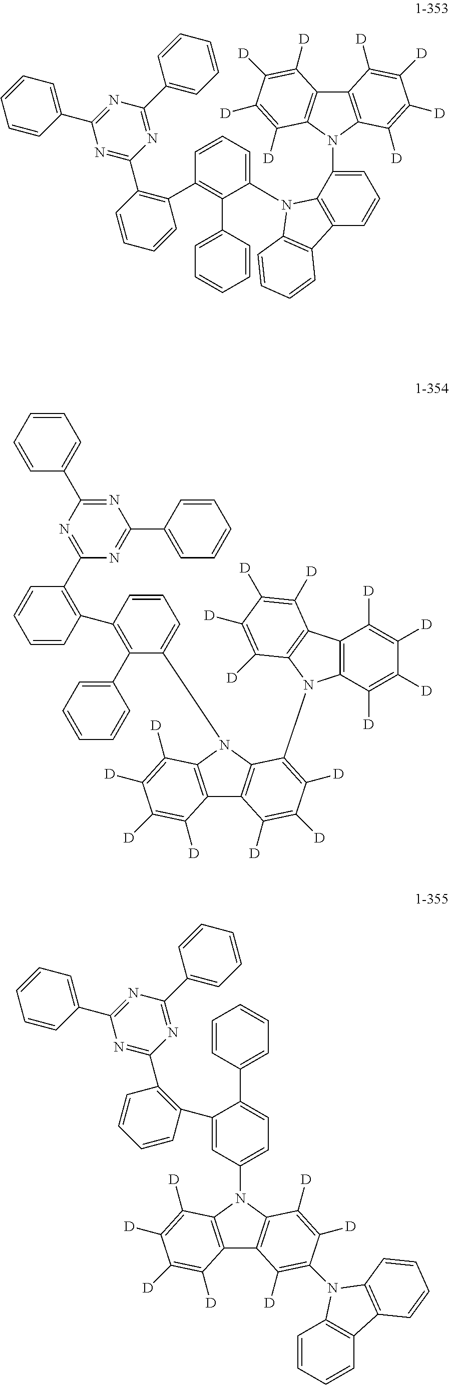 [SMILES: [2H]c1c([2H])c([2H])c2c(c1[2H])c1c([2H])c([2H])c([2H])c([2H])c1n2-c1cccc2c3ccccc3n(-c3cccc(-c4ccccc4-c4nc(-c5ccccc5)nc(-c5ccccc5)n4)c3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccccc2-c2nc(-c3ccccc3)nc(-c3ccccc3)n2)c1-c1ccccc1 ; [2H]c1c([2H])c([2H])c2c(c1[2H])c1c([2H])c(-n3c4ccccc4c4ccccc43)c([2H])c([2H])c1n2-c1ccc(-c2ccccc2)c(-c2ccccc2-c2nc(-c3ccccc3)nc(-c3ccccc3)n2)c1]

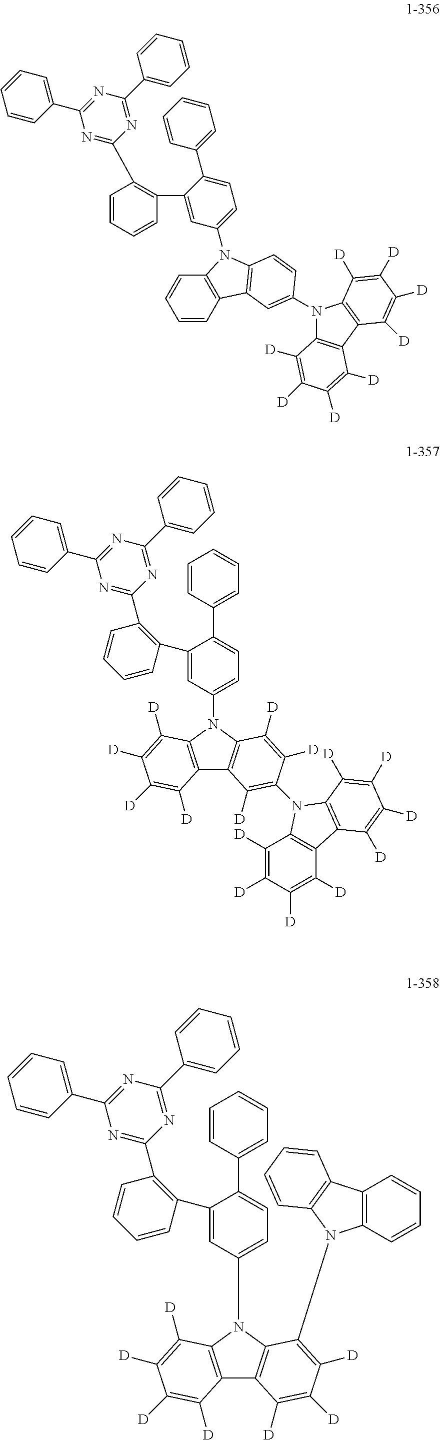 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2ccccc2)c(-c2ccccc2-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)c(-c2ccccc2-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1ccc(-c2ccccc2)c(-c2ccccc2-c2nc(-c3ccccc3)nc(-c3ccccc3)n2)c1]

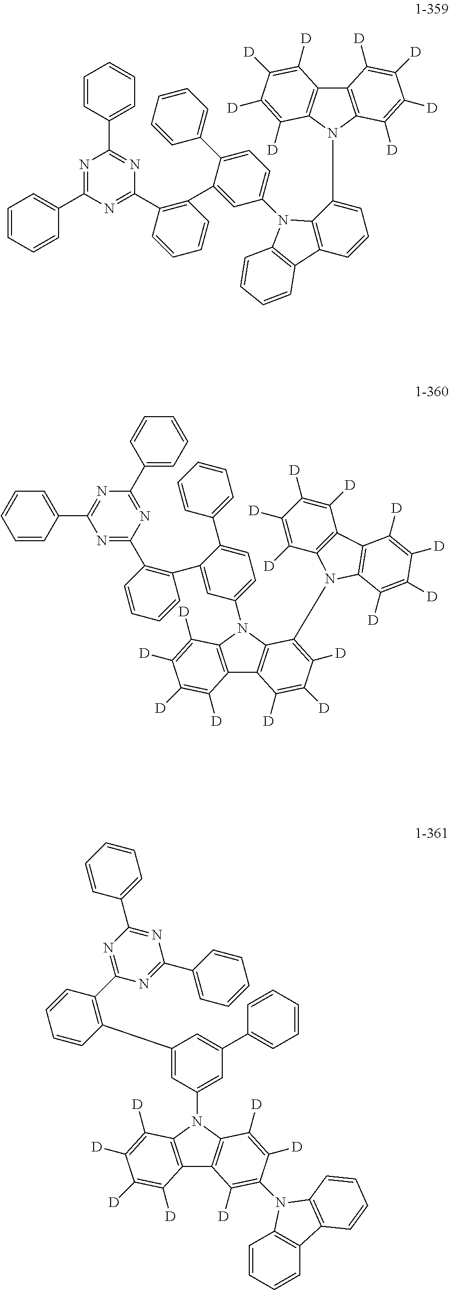 [SMILES: [2H]c1c([2H])c([2H])c2c(c1[2H])c1c([2H])c([2H])c([2H])c([2H])c1n2-c1cccc2c3ccccc3n(-c3ccc(-c4ccccc4)c(-c4ccccc4-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)c(-c2ccccc2-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cc(-c2ccccc2)cc(-c2ccccc2-c2nc(-c3ccccc3)nc(-c3ccccc3)n2)c1]

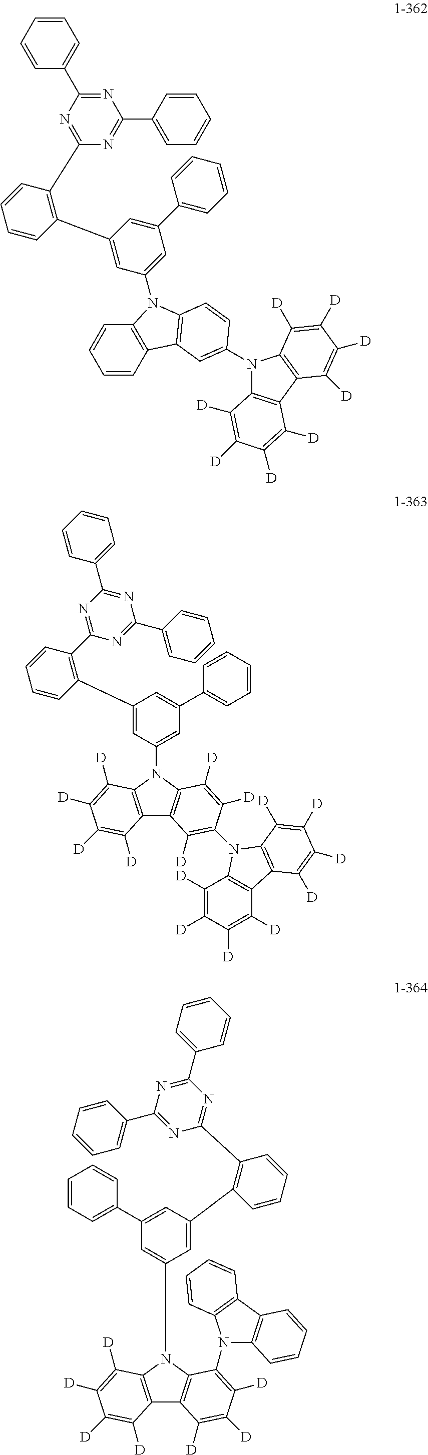 [SMILES: [2H]c1c([2H])c([2H])c2c(c1[2H])c1c([2H])c([2H])c([2H])c([2H])c1n2-c1ccc2c(c1)c1ccccc1n2-c1cc(-c2ccccc2)cc(-c2ccccc2-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)cc(-c2ccccc2-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cc(-c2ccccc2)cc(-c2ccccc2-c2nc(-c3ccccc3)nc(-c3ccccc3)n2)c1]

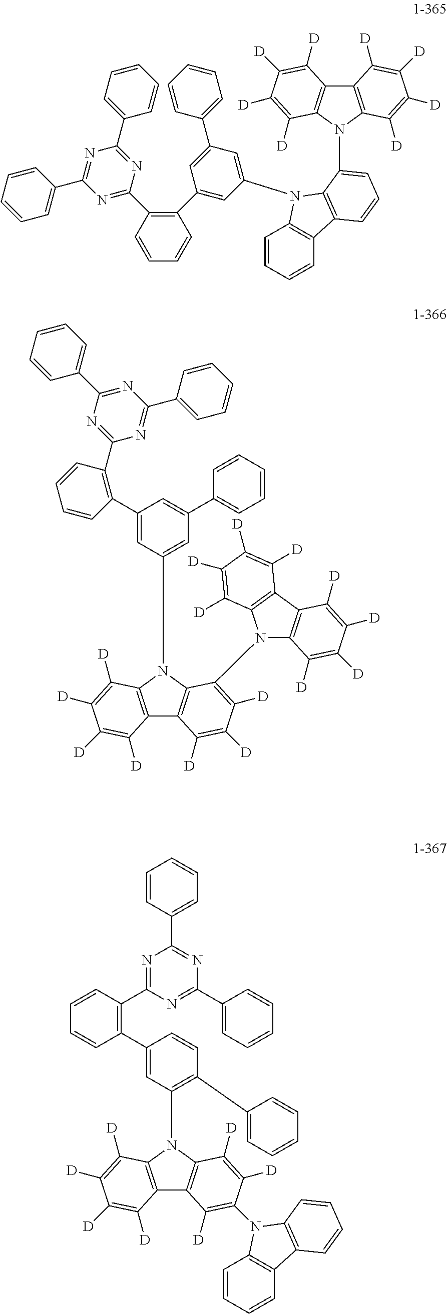

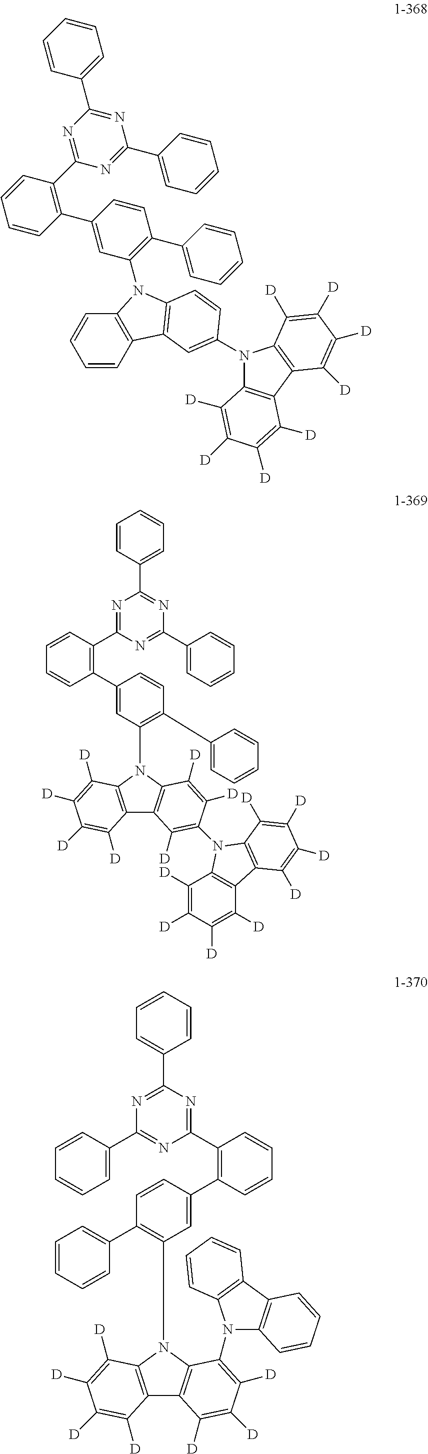 [SMILES: [2H]c1c([2H])c([2H])c2c(c1[2H])c1c([2H])c([2H])c([2H])c([2H])c1n2-c1ccc2c(c1)c1ccccc1n2-c1cc(-c2ccccc2-c2nc(-c3ccccc3)nc(-c3ccccc3)n2)ccc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2-c2nc(-c3ccccc3)nc(-c3ccccc3)n2)ccc1-c1ccccc1 ; [2H]c1c([2H])c([2H])c2c(c1[2H])c1c([2H])c([2H])c([2H])c(-n3c4ccccc4c4ccccc43)c1n2-c1cc(-c2ccccc2-c2nc(-c3ccccc3)nc(-c3ccccc3)n2)ccc1-c1ccccc1]

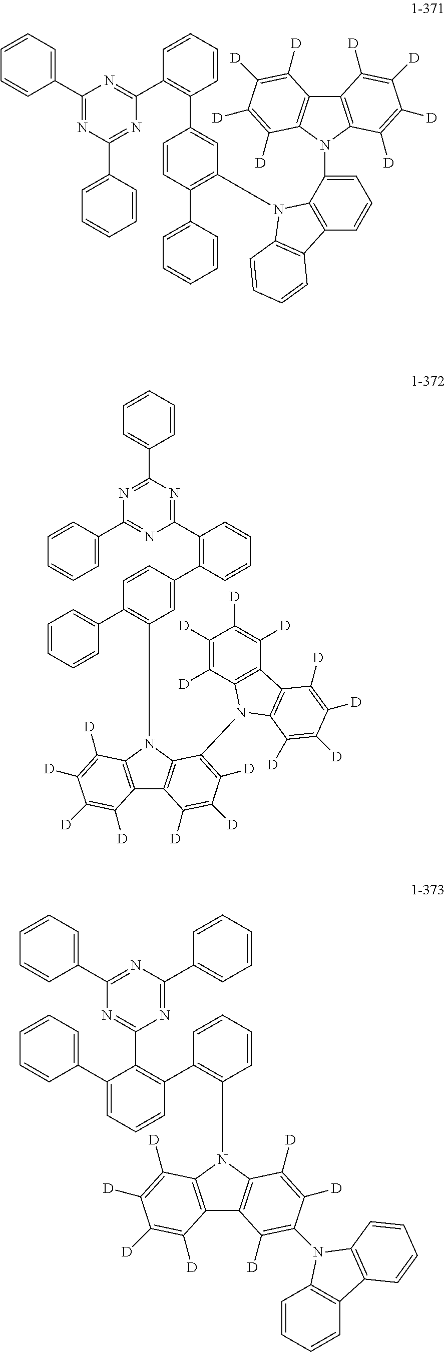 [SMILES: [2H]c1c([2H])c([2H])c2c(c1[2H])c1c([2H])c([2H])c([2H])c([2H])c1n2-c1cccc2c3ccccc3n(-c3cc(-c4ccccc4-c4nc(-c5ccccc5)nc(-c5ccccc5)n4)ccc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2-c2nc(-c3ccccc3)nc(-c3ccccc3)n2)ccc1-c1ccccc1 ; [2H]c1c([2H])c([2H])c2c(c1[2H])c1c([2H])c(-n3c4ccccc4c4ccccc43)c([2H])c([2H])c1n2-c1ccccc1-c1cccc(-c2ccccc2)c1-c1nc(-c2ccccc2)nc(-c2ccccc2)n1]

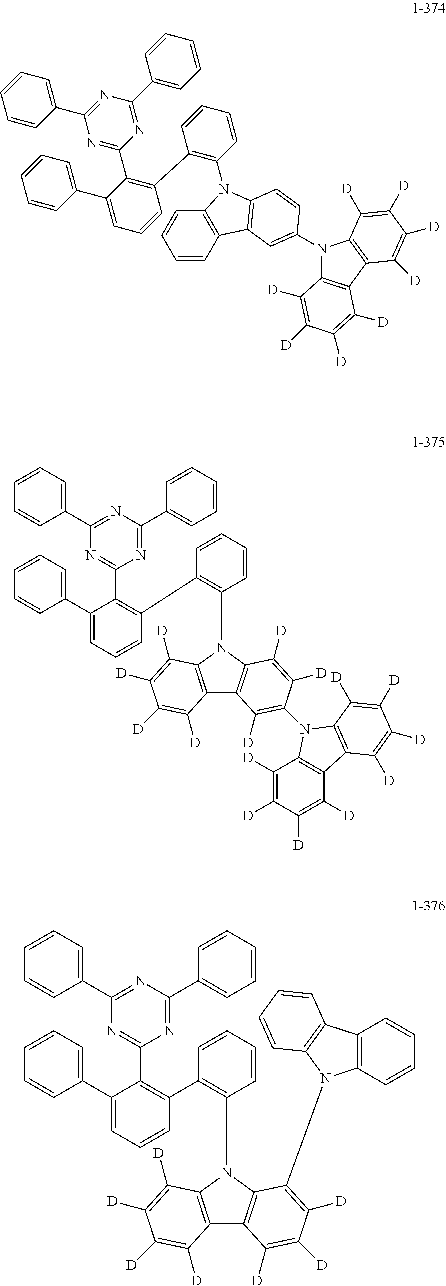 [SMILES: [2H]c1c([2H])c([2H])c2c(c1[2H])c1c([2H])c([2H])c([2H])c([2H])c1n2-c1ccc2c(c1)c1ccccc1n2-c1ccccc1-c1cccc(-c2ccccc2)c1-c1nc(-c2ccccc2)nc(-c2ccccc2)n1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1cccc(-c2ccccc2)c1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1ccccc1-c1cccc(-c2ccccc2)c1-c1nc(-c2ccccc2)nc(-c2ccccc2)n1]

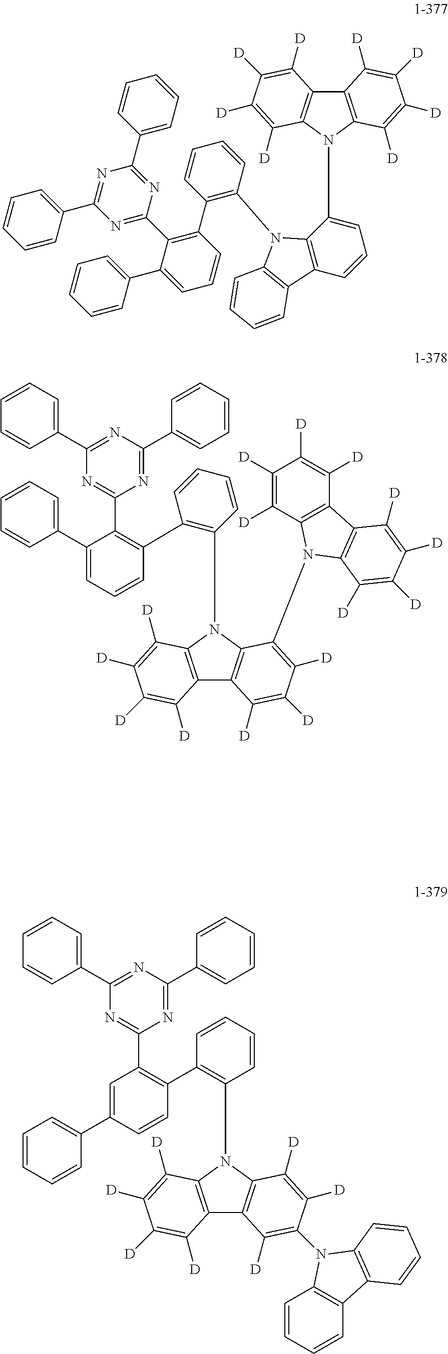 [SMILES: [2H]c1c([2H])c([2H])c2c(c1[2H])c1c([2H])c([2H])c([2H])c([2H])c1n2-c1cccc2c3ccccc3n(-c3ccccc3-c3cccc(-c4ccccc4)c3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1cccc(-c2ccccc2)c1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1ccccc1-c1ccc(-c2ccccc2)cc1-c1nc(-c2ccccc2)nc(-c2ccccc2)n1]

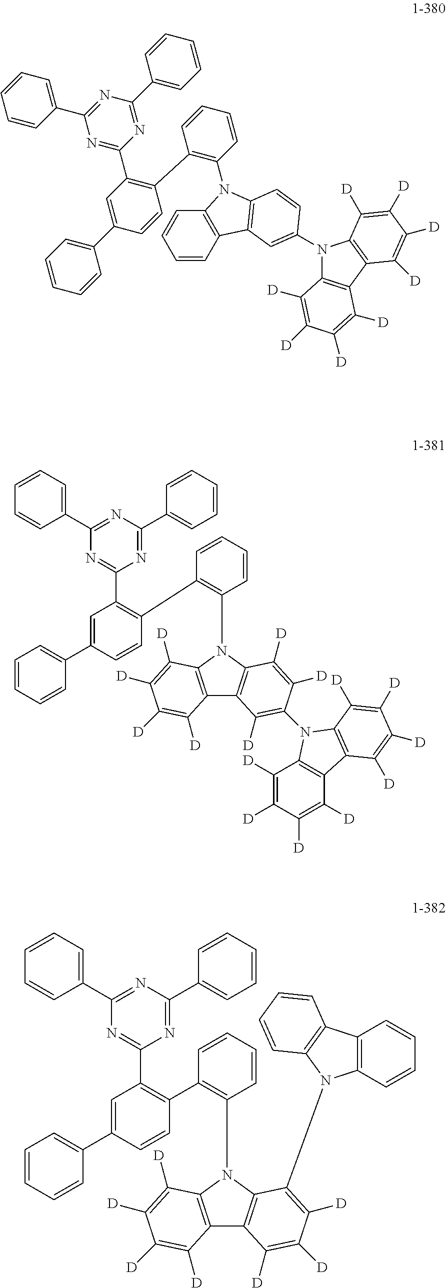 [SMILES: [2H]c1c([2H])c([2H])c2c(c1[2H])c1c([2H])c([2H])c([2H])c([2H])c1n2-c1ccc2c(c1)c1ccccc1n2-c1ccccc1-c1ccc(-c2ccccc2)cc1-c1nc(-c2ccccc2)nc(-c2ccccc2)n1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1ccc(-c2ccccc2)cc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1ccccc1-c1ccc(-c2ccccc2)cc1-c1nc(-c2ccccc2)nc(-c2ccccc2)n1]

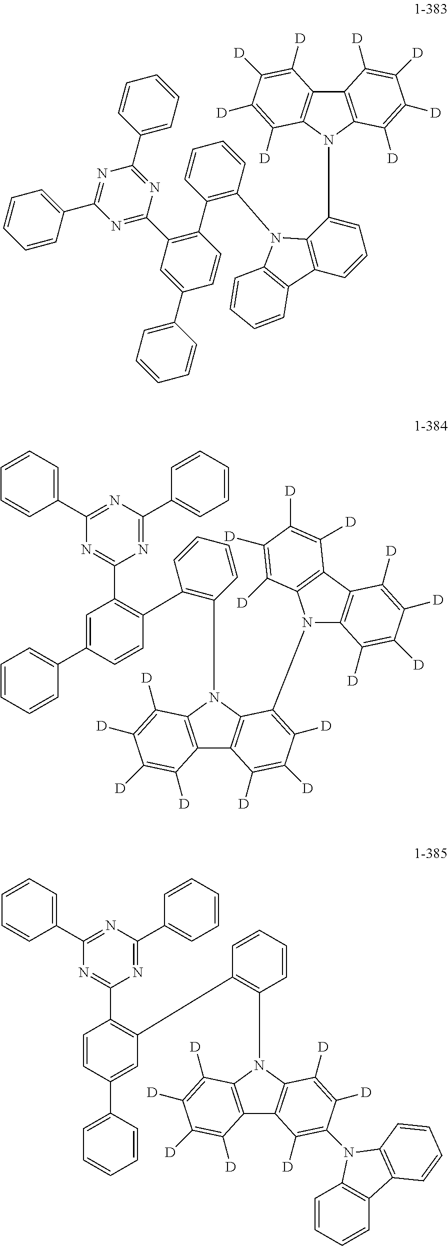 [SMILES: [2H]c1c([2H])c([2H])c2c(c1[2H])c1c([2H])c([2H])c([2H])c([2H])c1n2-c1cccc2c3ccccc3n(-c3ccccc3-c3ccc(-c4ccccc4)cc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1ccc(-c2ccccc2)cc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1ccccc1-c1cc(-c2ccccc2)ccc1-c1nc(-c2ccccc2)nc(-c2ccccc2)n1]

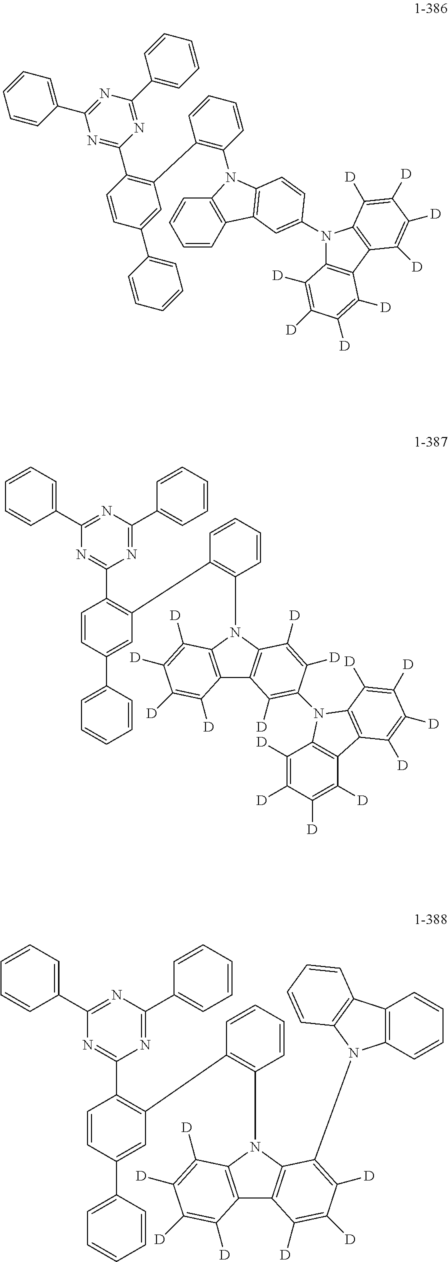 [SMILES: [2H]c1c([2H])c([2H])c2c(c1[2H])c1c([2H])c([2H])c([2H])c([2H])c1n2-c1ccc2c(c1)c1ccccc1n2-c1ccccc1-c1cc(-c2ccccc2)ccc1-c1nc(-c2ccccc2)nc(-c2ccccc2)n1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1cc(-c2ccccc2)ccc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1ccccc1-c1cc(-c2ccccc2)ccc1-c1nc(-c2ccccc2)nc(-c2ccccc2)n1]

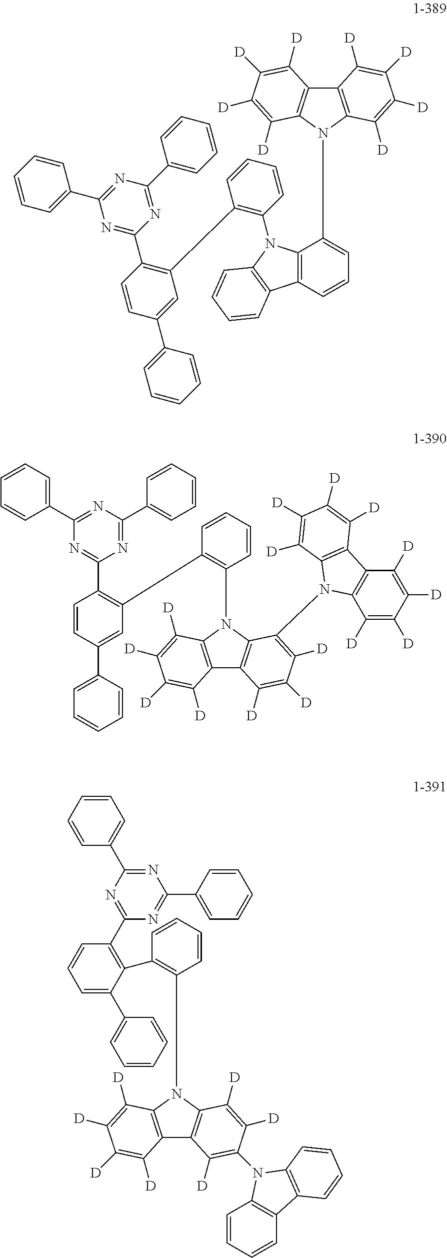 [SMILES: [2H]c1c([2H])c([2H])c2c(c1[2H])c1c([2H])c([2H])c([2H])c([2H])c1n2-c1cccc2c3ccccc3n(-c3ccccc3-c3cc(-c4ccccc4)ccc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1cc(-c2ccccc2)ccc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1ccccc1-c1c(-c2ccccc2)cccc1-c1nc(-c2ccccc2)nc(-c2ccccc2)n1]

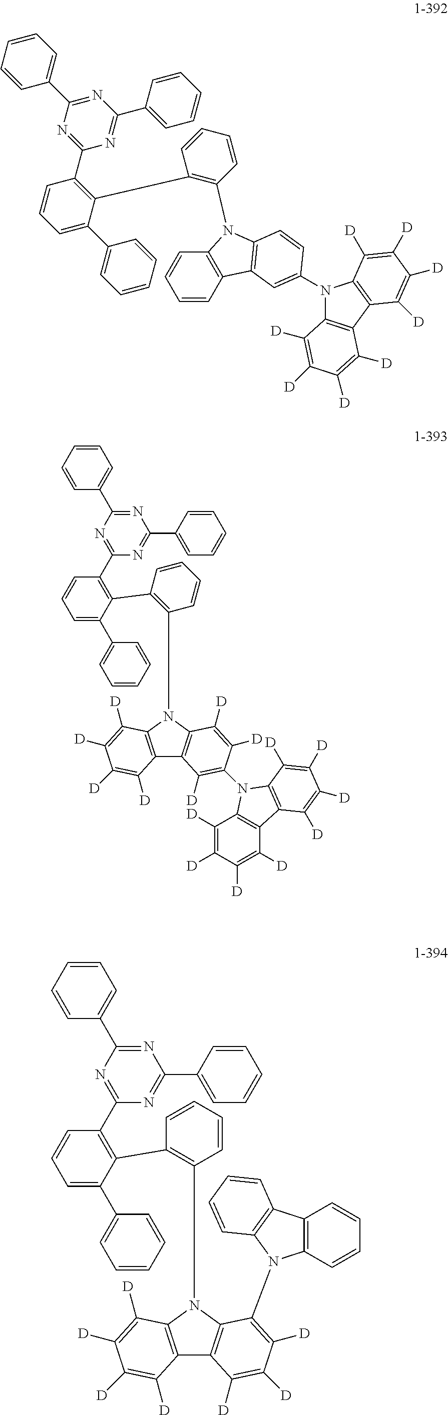 [SMILES: [2H]c1c([2H])c([2H])c2c(c1[2H])c1c([2H])c([2H])c([2H])c([2H])c1n2-c1ccc2c(c1)c1ccccc1n2-c1ccccc1-c1c(-c2ccccc2)cccc1-c1nc(-c2ccccc2)nc(-c2ccccc2)n1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1c(-c2ccccc2)cccc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1ccccc1-c1c(-c2ccccc2)cccc1-c1nc(-c2ccccc2)nc(-c2ccccc2)n1]

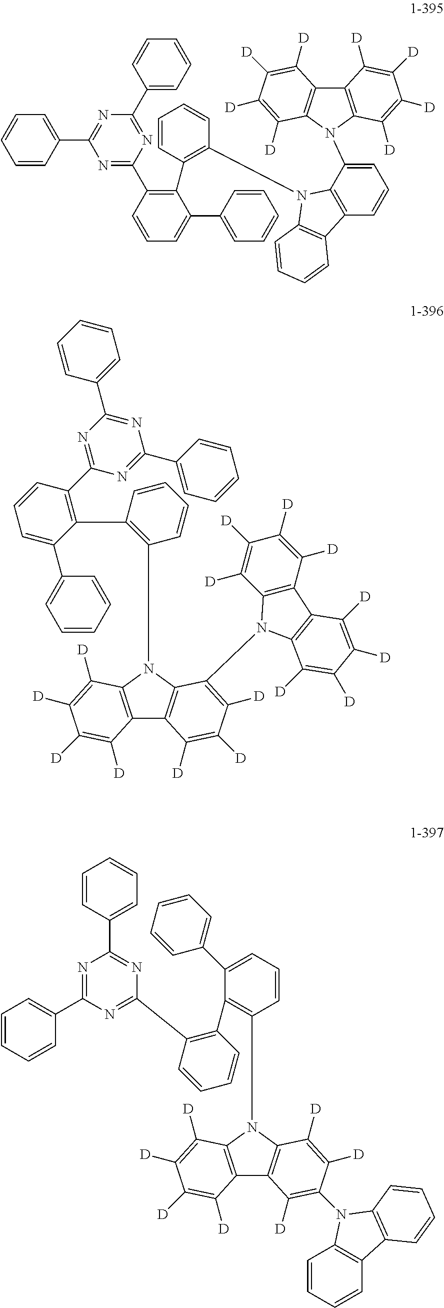 [SMILES: [2H]c1c([2H])c([2H])c2c(c1[2H])c1c([2H])c([2H])c([2H])c([2H])c1n2-c1cccc2c3ccccc3n(-c3ccccc3-c3c(-c4ccccc4)cccc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1c(-c2ccccc2)cccc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1cccc(-c2ccccc2)c1-c1ccccc1-c1nc(-c2ccccc2)nc(-c2ccccc2)n1]

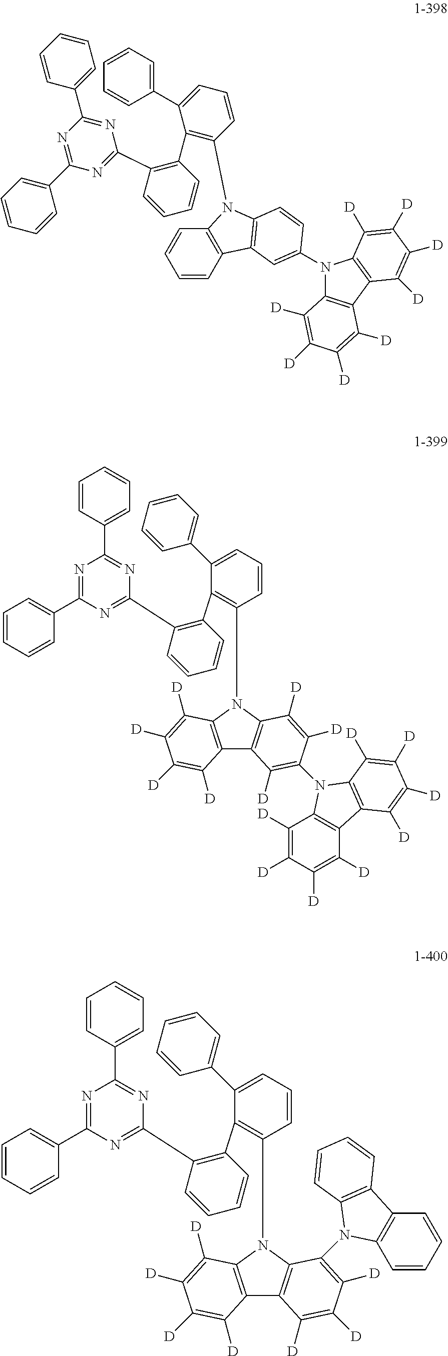

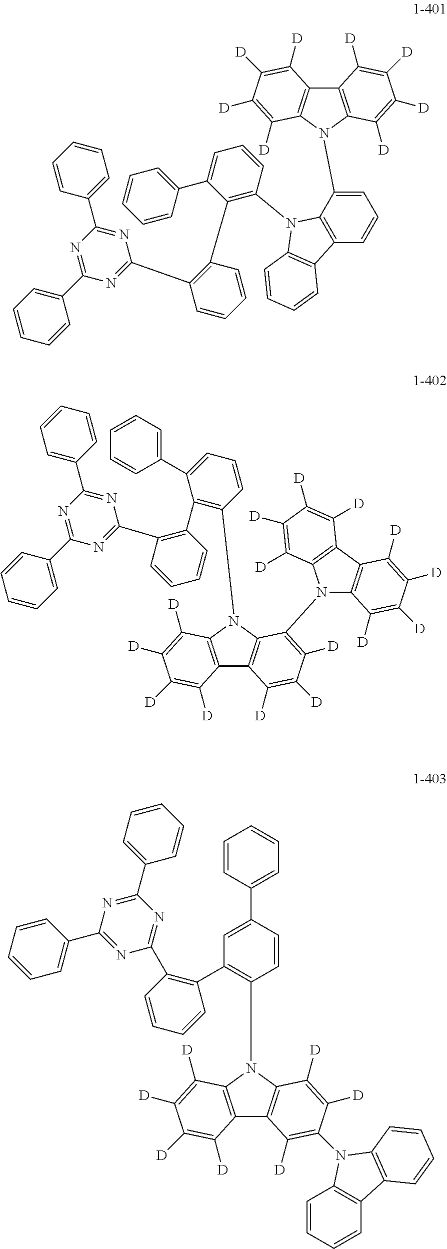 [SMILES: [2H]c1c([2H])c([2H])c2c(c1[2H])c1c([2H])c([2H])c([2H])c([2H])c1n2-c1cccc2c3ccccc3n(-c3cccc(-c4ccccc4)c3-c3ccccc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccccc2)c1-c1ccccc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1ccc(-c2ccccc2)cc1-c1ccccc1-c1nc(-c2ccccc2)nc(-c2ccccc2)n1]

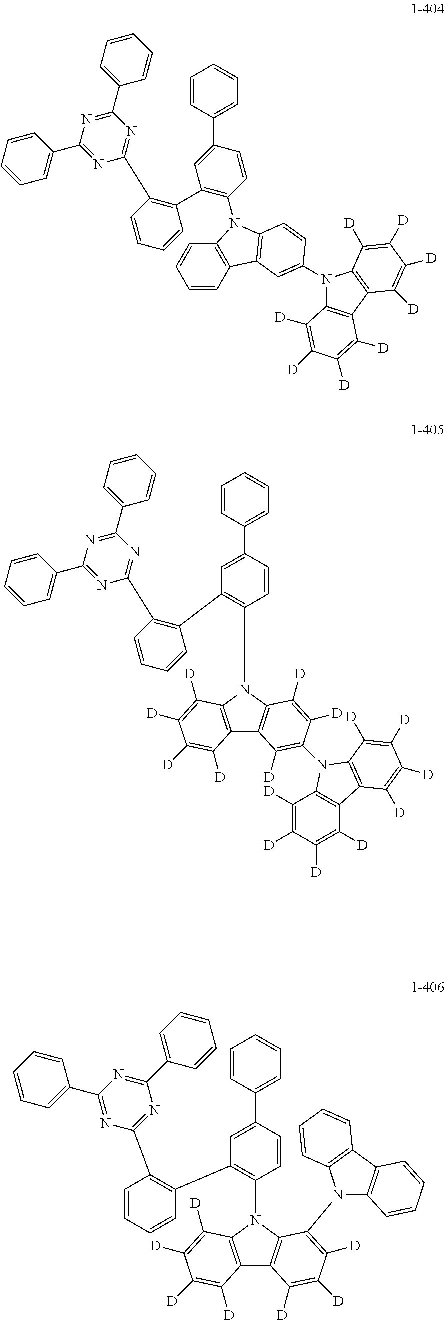

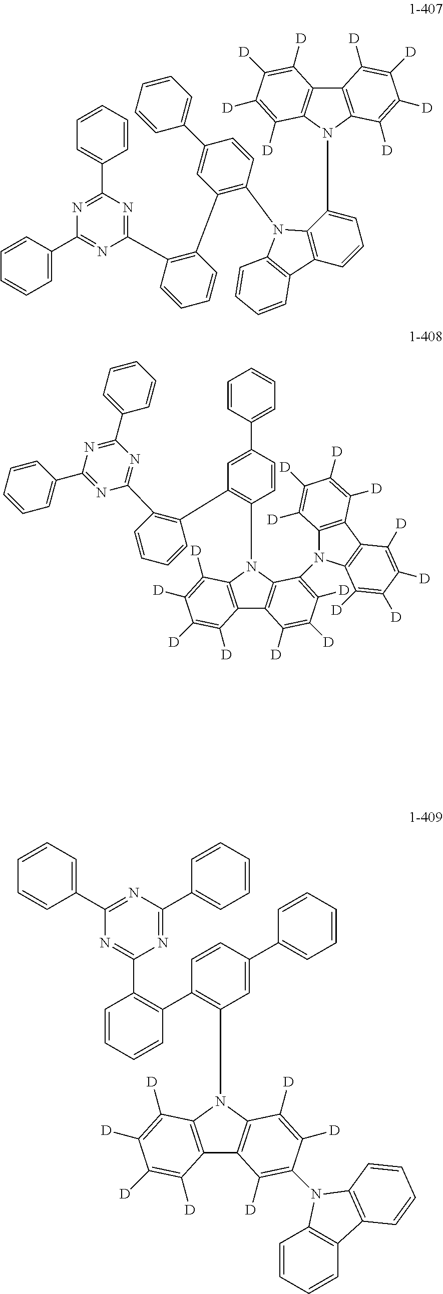 [SMILES: [2H]c1c([2H])c([2H])c2c(c1[2H])c1c([2H])c([2H])c([2H])c([2H])c1n2-c1cccc2c3ccccc3n(-c3ccc(-c4ccccc4)cc3-c3ccccc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)cc1-c1ccccc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1cc(-c2ccccc2)ccc1-c1ccccc1-c1nc(-c2ccccc2)nc(-c2ccccc2)n1]

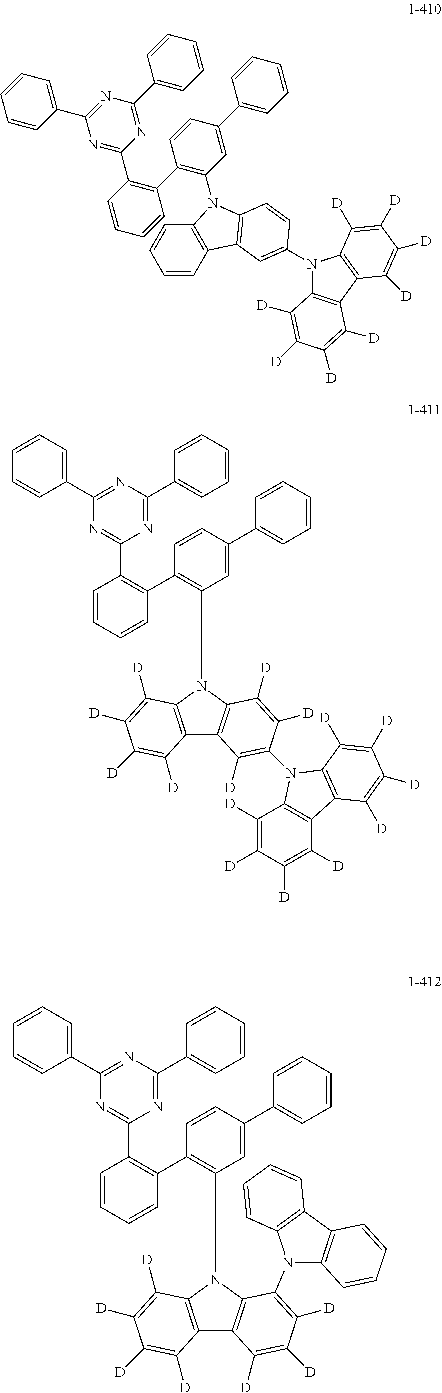

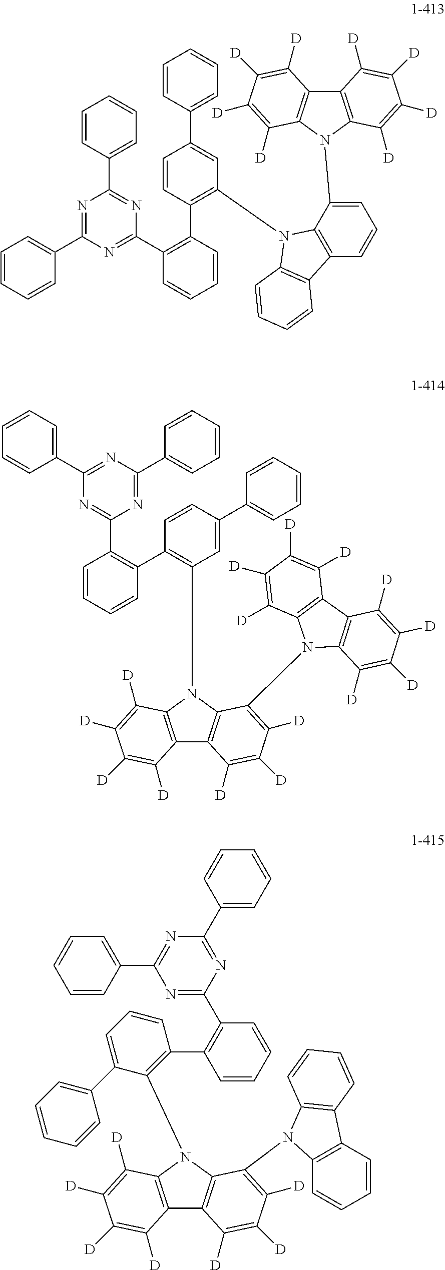 [SMILES: [2H]c1c([2H])c([2H])c2c(c1[2H])c1c([2H])c([2H])c([2H])c([2H])c1n2-c1cccc2c3ccccc3n(-c3cc(-c4ccccc4)ccc3-c3ccccc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2)ccc1-c1ccccc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1c(-c2ccccc2)cccc1-c1ccccc1-c1nc(-c2ccccc2)nc(-c2ccccc2)n1]

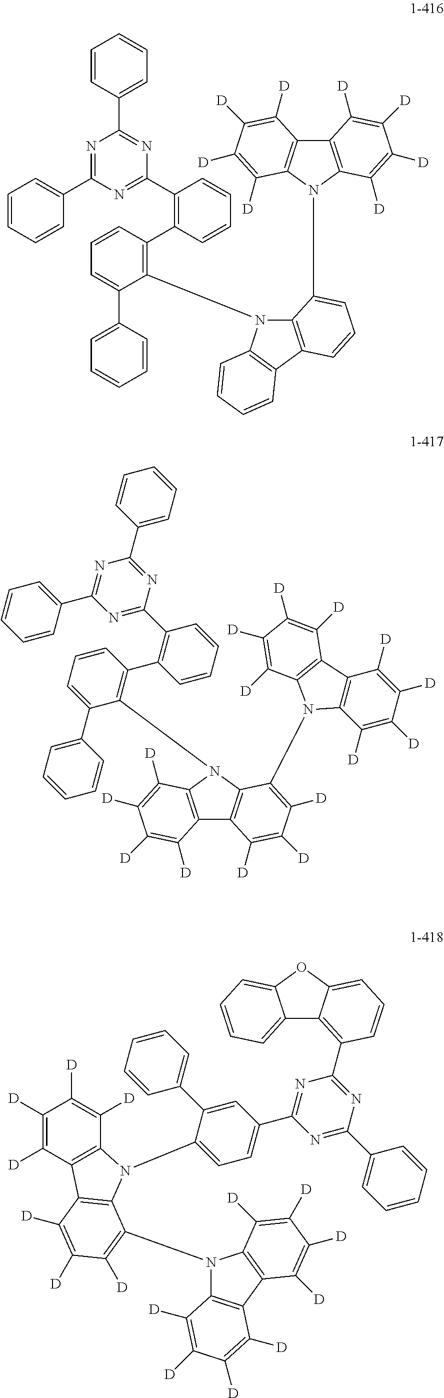

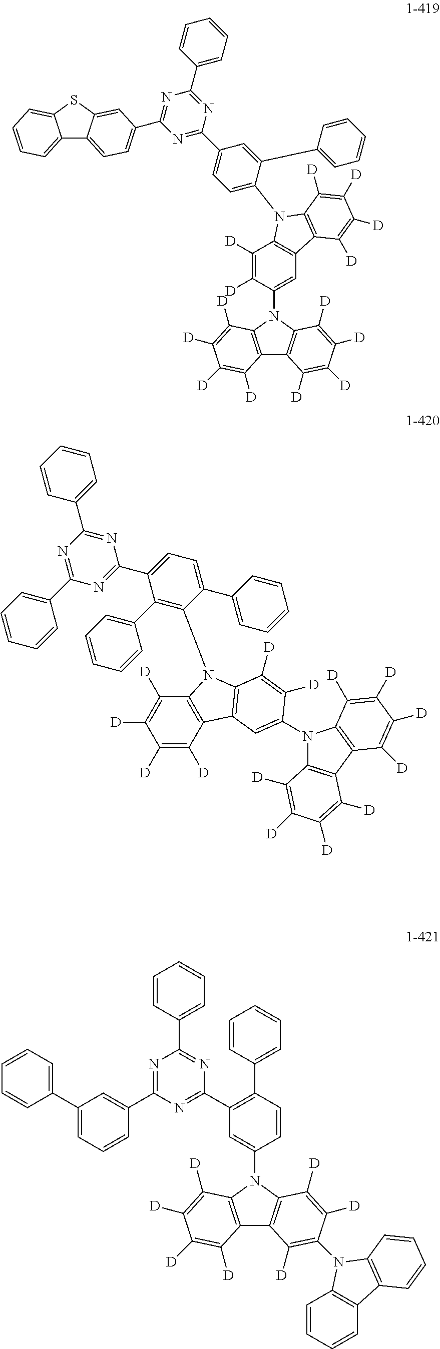

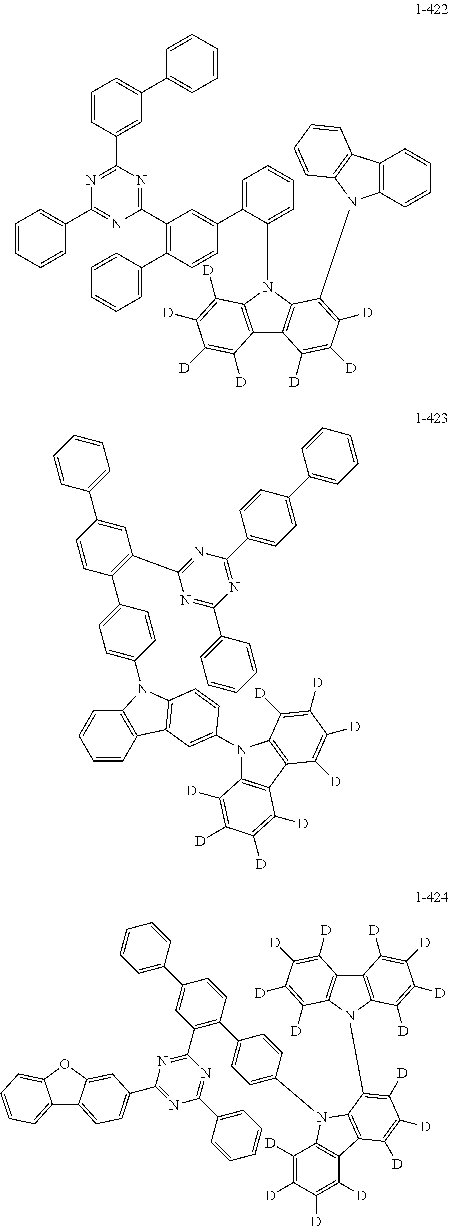

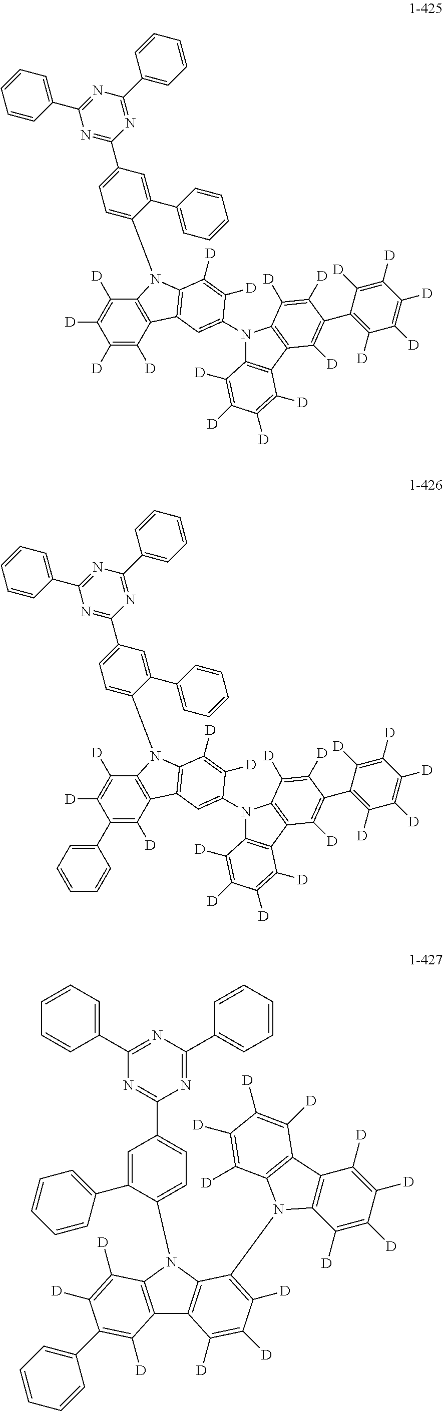

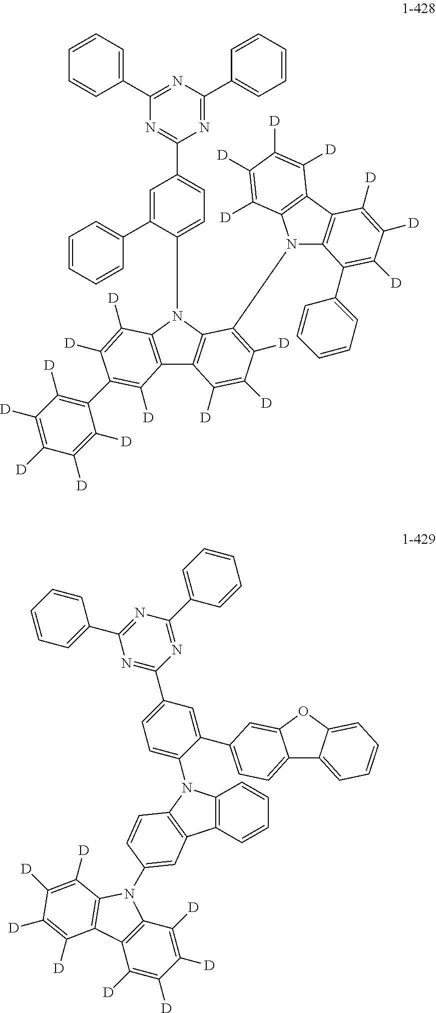 [SMILES: [2H]c1c([2H])c([2H])c(-c2c([2H])c([2H])c3c(c2[2H])c2c([2H])c([2H])c([2H])c(-n4c5c([2H])c([2H])c([2H])c([2H])c5c5c([2H])c([2H])c([2H])c(-c6ccccc6)c54)c2n3-c2ccc(-c3nc(-c4ccccc4)nc(-c4ccccc4)n3)cc2-c2ccccc2)c([2H])c1[2H] ; [2H]c1c([2H])c([2H])c2c(c1[2H])c1c([2H])c([2H])c([2H])c([2H])c1n2-c1ccc2c(c1)c1ccccc1n2-c1ccc(-c2nc(-c3ccccc3)nc(-c3ccccc3)n2)cc1-c1ccc2c(c1)oc1ccccc12]

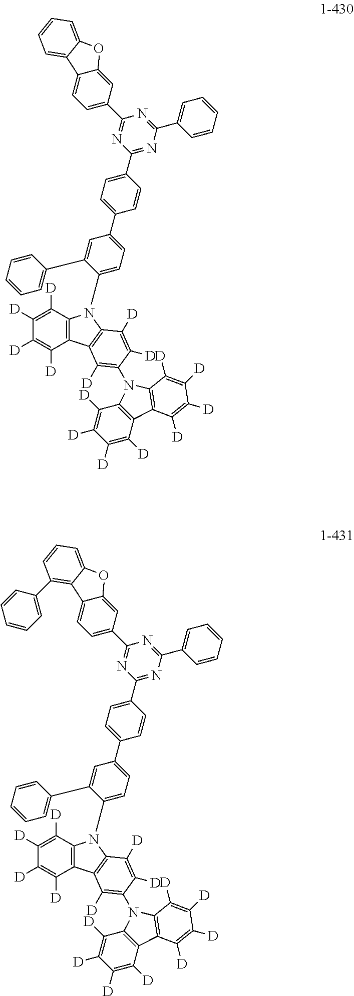 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccc5c(c4)oc4ccccc45)n3)cc2)cc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccc5c(c4)oc4cccc(-c6ccccc6)c45)n3)cc2)cc1-c1ccccc1]

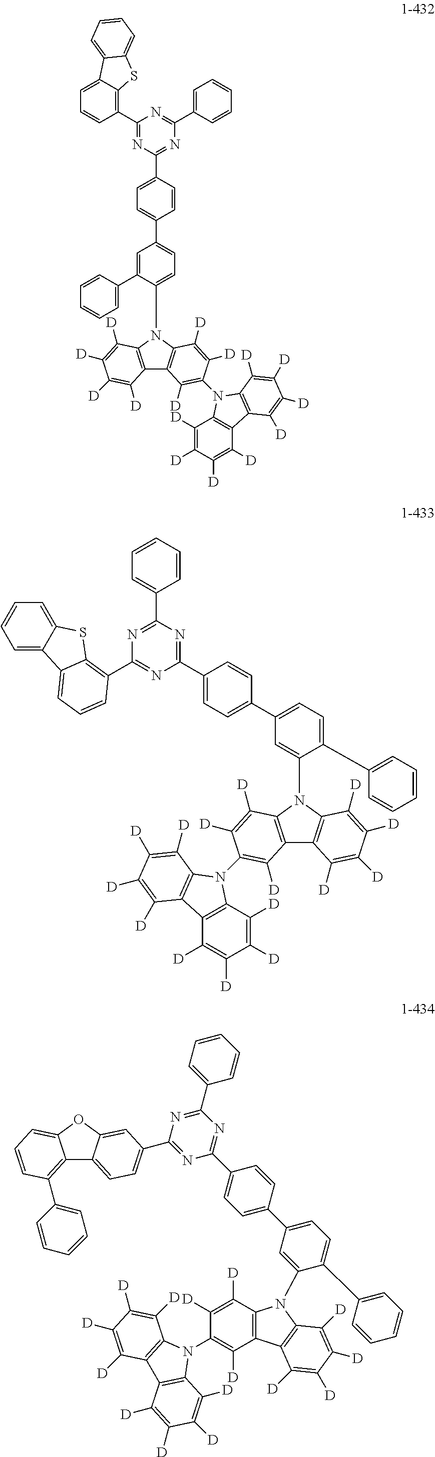

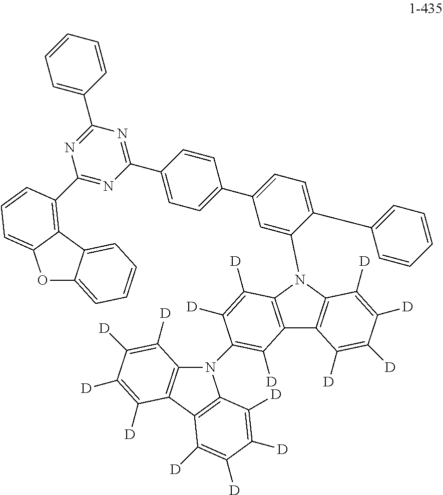 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4cccc5oc6ccccc6c45)n3)cc2)ccc1-c1ccccc1]

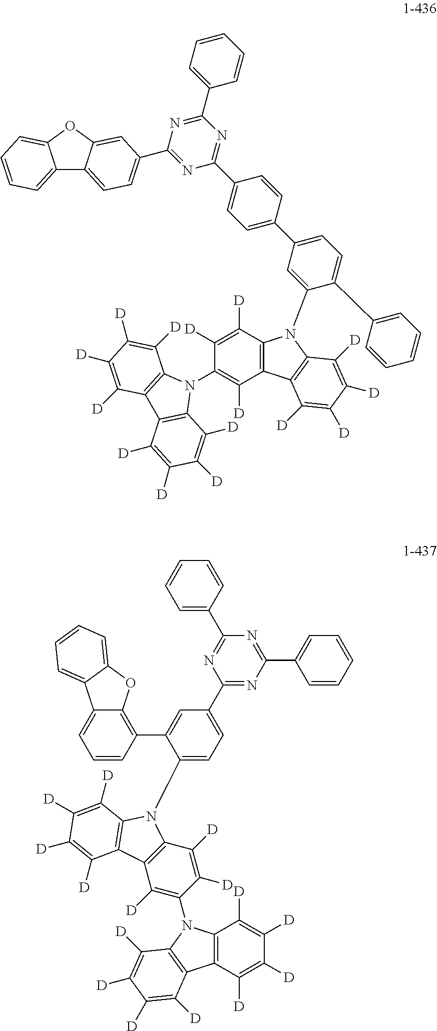 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4ccc5c(c4)oc4ccccc45)n3)cc2)ccc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2nc(-c3ccccc3)nc(-c3ccccc3)n2)cc1-c1cccc2c1oc1ccccc12]

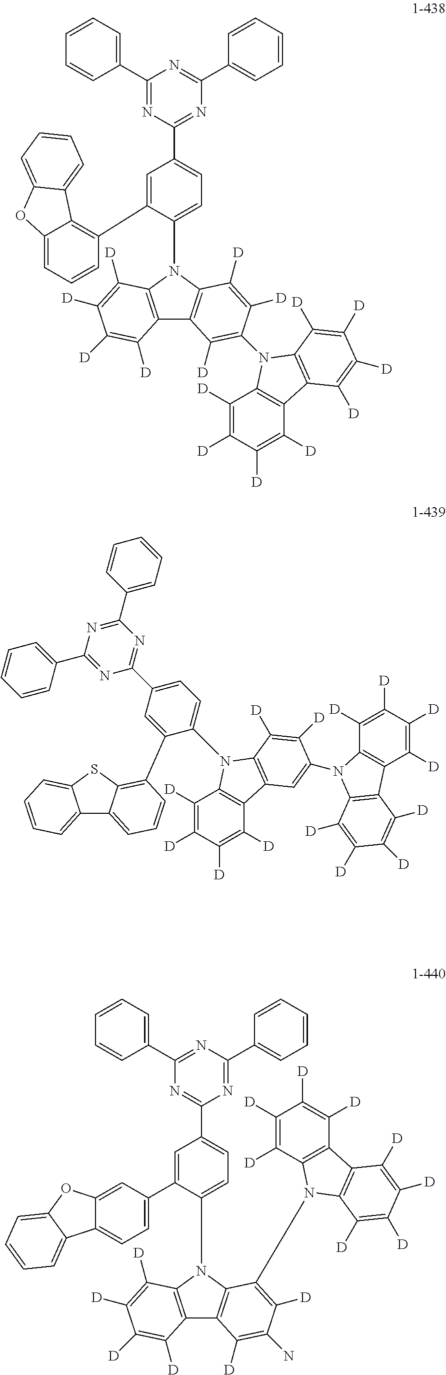

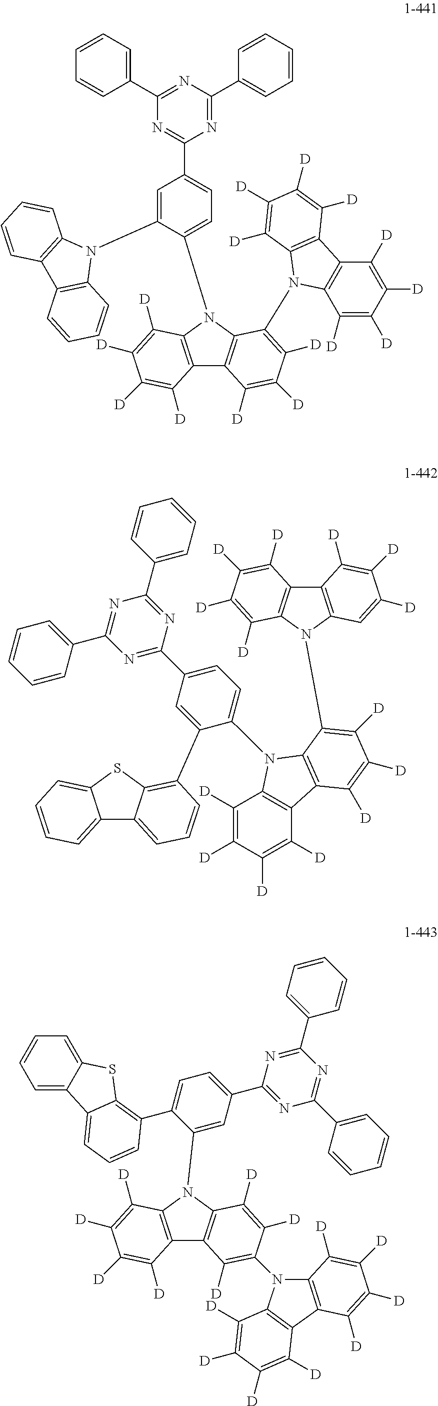

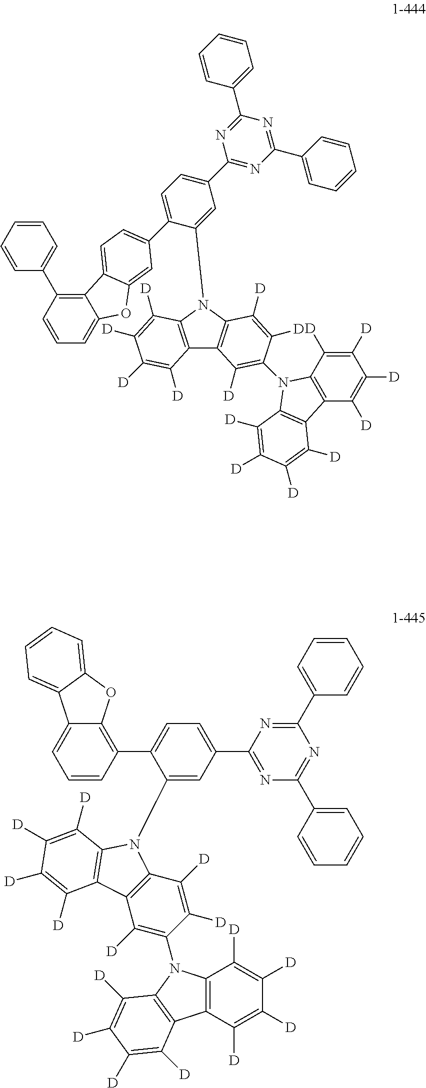 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2nc(-c3ccccc3)nc(-c3ccccc3)n2)ccc1-c1ccc2c(c1)oc1cccc(-c3ccccc3)c12 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2nc(-c3ccccc3)nc(-c3ccccc3)n2)ccc1-c1cccc2c1oc1ccccc12]

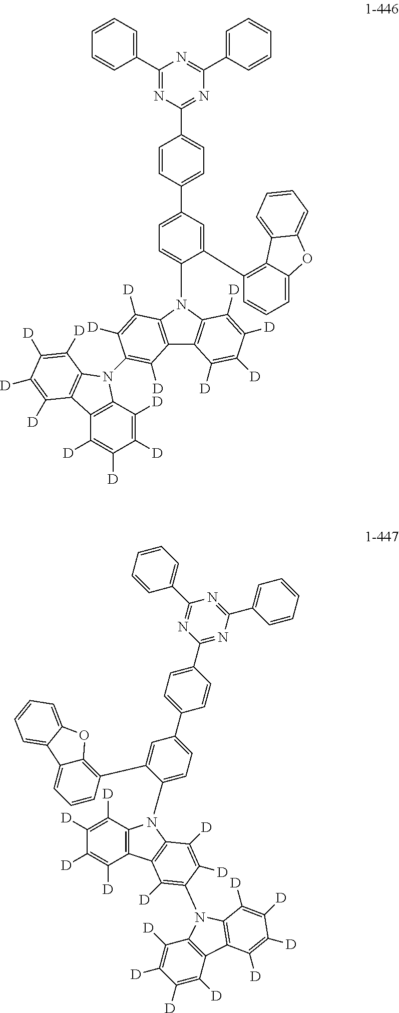 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)cc1-c1cccc2oc3ccccc3c12 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)cc1-c1cccc2c1oc1ccccc12]

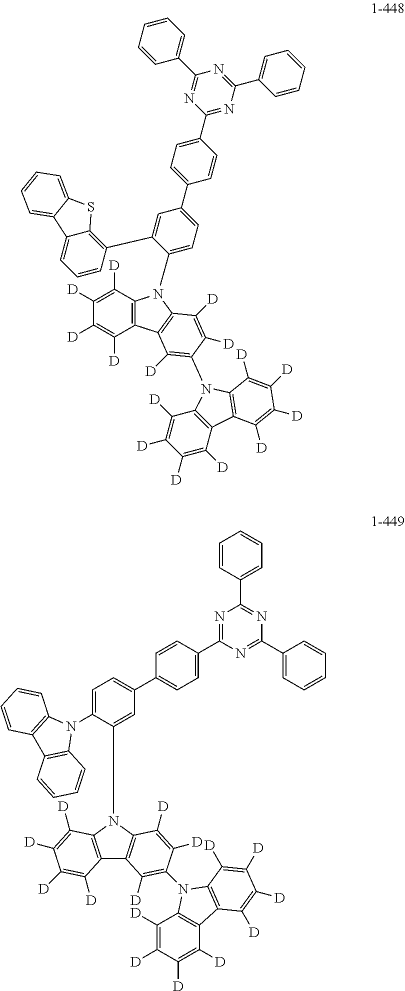 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)cc1-c1cccc2c1sc1ccccc12 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4ccccc4)n3)cc2)ccc1-n1c2ccccc2c2ccccc21]

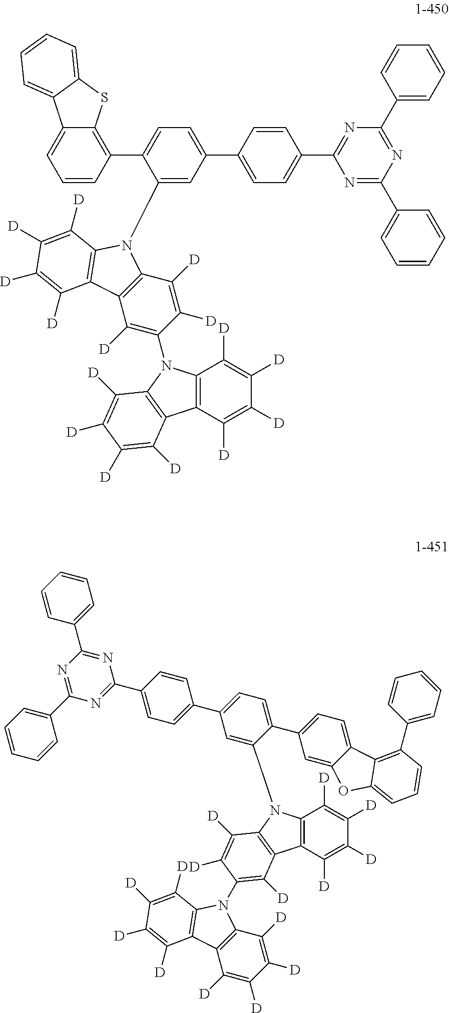 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4ccccc4)n3)cc2)ccc1-c1cccc2c1sc1ccccc12 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4ccccc4)n3)cc2)ccc1-c1ccc2c(c1)oc1cccc(-c3ccccc3)c12]

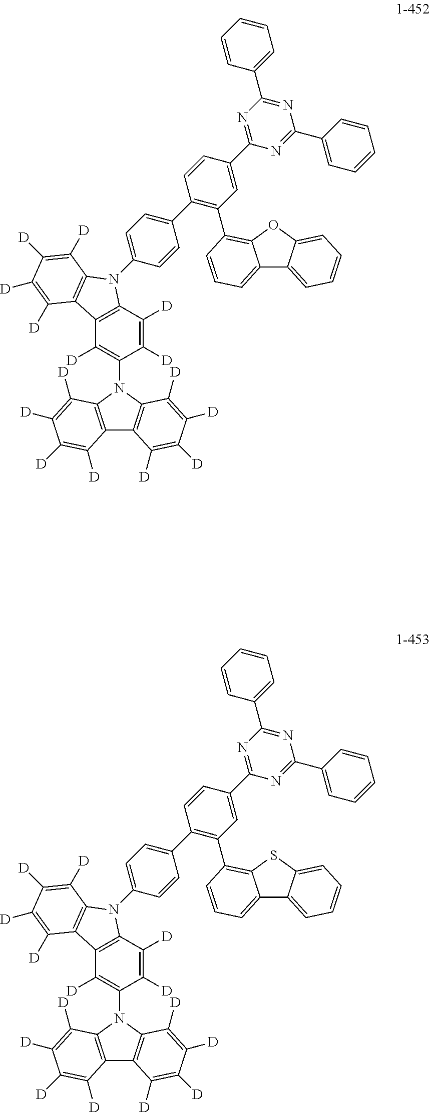 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2-c2cccc3c2oc2ccccc23)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2-c2cccc3c2sc2ccccc23)cc1]

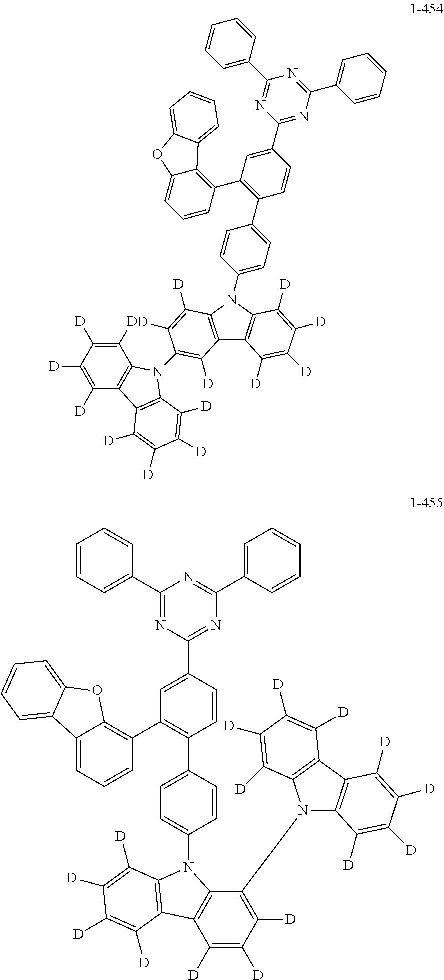 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2-c2cccc3oc4ccccc4c23)cc1 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)cc2-c2cccc3c2oc2ccccc23)cc1]

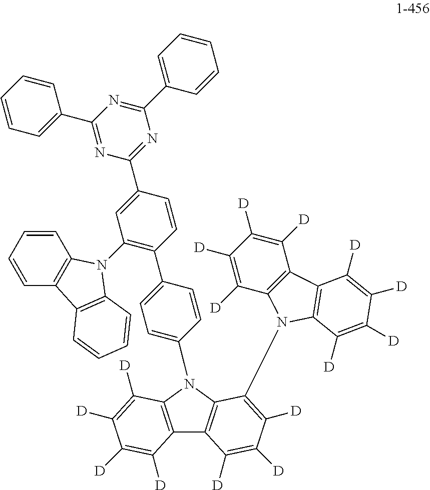 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)cc2-n2c3ccccc3c3ccccc32)cc1]

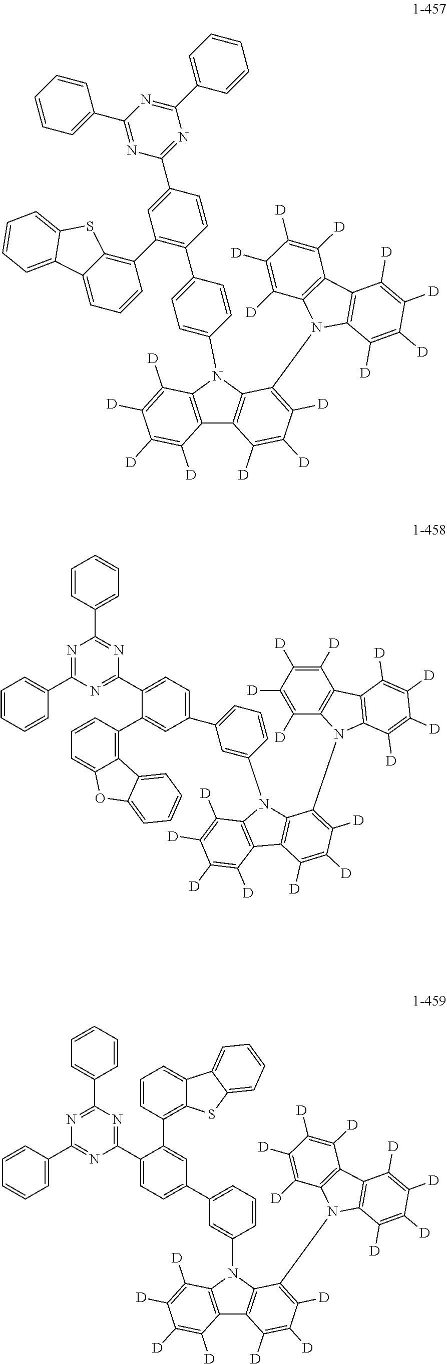

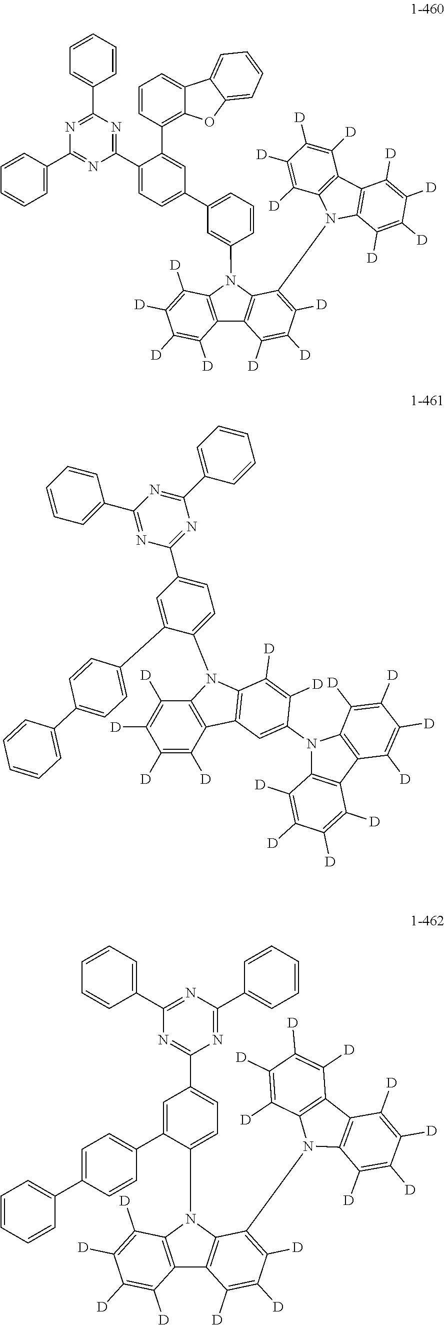

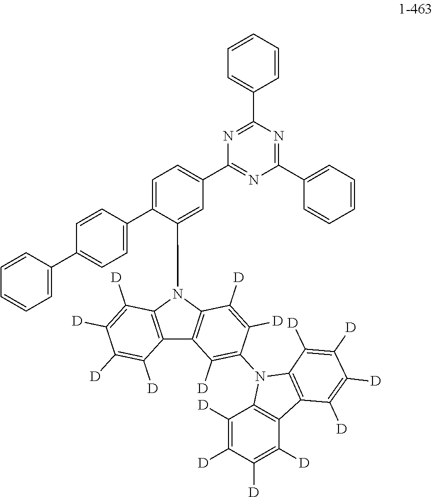 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2nc(-c3ccccc3)nc(-c3ccccc3)n2)ccc1-c1ccc(-c2ccccc2)cc1]

In an exemplary embodiment of the present application, the compound is an example, and is not limited thereto, and may include other compounds included in Chemical Formula 1 comprising an additional substituent. In addition, regarding a substitution position of deuterium of the compound, a specific position may be excluded during a deuterium substitution and synthesis process, and hydrogen and deuterium may exist in a mixed state.

In addition, by introducing various substituents into the structure of Chemical Formula 1, compounds having unique characteristics of the introduced substituents may be synthesized. For example, by introducing a substituent mainly used in a hole injection material, a hole transport material, a light-emitting material, an electron transport material, and an electron injection material used in manufacturing an organic light-emitting device into the core structure, a material satisfying conditions required for each organic material layer may be synthesized.

In addition, by introducing various substituents into the structure of Chemical Formula 1 or changing a bonding position, a band gap may be finely adjusted, and on the other hand, characteristics at an interface between organic material layers may be improved.

In addition, the compound of Chemical Formula 1 has excellent thermal stability, and such thermal stability provides driving stability to the organic light-emitting device and improves lifespan characteristics.

In an exemplary embodiment of the present application, an organic light-emitting device comprising: a first electrode; a second electrode provided to face the first electrode; and one or more organic material layers provided between the first electrode and the second electrode, wherein one or more layers of the organic material layers comprise the heterocyclic compound represented by Chemical Formula 1, is provided.

In another exemplary embodiment, an organic light-emitting device comprising: a first electrode; a second electrode provided to face the first electrode; and one or more organic material layers provided between the first electrode and the second electrode, wherein one or more layers of the organic material layers comprise one kind of the heterocyclic compound represented by Chemical Formula 1, is provided.

In another exemplary embodiment, an organic light-emitting device comprising: a first electrode; a second electrode provided to face the first electrode; and one or more organic material layers provided between the first electrode and the second electrode, wherein one or more layers of the organic material layers comprise two or more kinds of the heterocyclic compounds represented by Chemical Formula 1, is provided.

In another exemplary embodiment, the heterocyclic compound represented by Chemical Formula 1 may be used as a light-emitting material of a light-emitting layer of an organic light-emitting device.

In another exemplary embodiment, the heterocyclic compound represented by Chemical Formula 1 may be used as a host material of a light-emitting layer of an organic light-emitting device.

In an exemplary embodiment of the present application, the first electrode may be an anode, and the second electrode may be a cathode.

In another exemplary embodiment, the first electrode may be a cathode, and the second electrode may be an anode.

In an exemplary embodiment of the present application, the organic light-emitting device may be a blue organic light-emitting device, and the heterocyclic compound according to Chemical Formula 1 may be used as a material for the blue organic light-emitting device.

In an exemplary embodiment of the present application, the organic light-emitting device may be a green organic light-emitting device, and the heterocyclic compound represented by Chemical Formula 1 may be used as a material for the green organic light-emitting device.

In an exemplary embodiment of the present application, the organic light-emitting device may be a red organic light-emitting device, and the heterocyclic compound represented by Chemical Formula 1 may be used as a material for the red organic light-emitting device.

In an exemplary embodiment of the present application, the organic light-emitting device may be a blue organic light-emitting device, and the heterocyclic compound according to Chemical Formula 1 may be used as a material for a light-emitting layer of the blue organic light-emitting device.

In an exemplary embodiment of the present application, the organic light-emitting device may be a green organic light-emitting device, and the heterocyclic compound represented by Chemical Formula 1 may be used as a material for a light-emitting layer of the green organic light-emitting device.

In an exemplary embodiment of the present application, the organic light-emitting device may be a red organic light-emitting device, and the heterocyclic compound represented by Chemical Formula 1 may be used as a material for a light-emitting layer of the red organic light-emitting device.

In an exemplary embodiment of the present application, specific details regarding the heterocyclic compound represented by Chemical Formula 1 are the same as described above.

The organic light-emitting device of the present invention may be manufactured using a conventional manufacturing method and material for an organic light-emitting device, except that one or more organic material layers are formed using the aforementioned heterocyclic compound.

The heterocyclic compound may be formed into an organic material layer by a solution coating method as well as a vacuum deposition method when manufacturing the organic light-emitting device. Here, the solution coating method means spin coating, dip coating, inkjet printing, screen printing, a spray method, roll coating, and the like, but is not limited thereto.

The organic material layer of the organic light-emitting device of the present invention may have a single-layer structure, or may have a multi-layer structure in which two or more organic material layers are stacked. For example, the organic light-emitting device of the present invention may have a structure comprising a hole injection layer, a hole transport layer, a light-emitting layer, an electron transport layer, an electron injection layer, and the like as the organic material layer. However, the structure of the organic light-emitting device is not limited thereto, and may comprise a smaller number of organic material layers.

In an exemplary embodiment of the present application, Ir(ppy)₃, which is a green phosphorescent dopant, may be used as an iridium-based dopant.

In an exemplary embodiment of the present application, (piq)₂(Ir)(acac), which is a red phosphorescent dopant, may be used as the iridium-based dopant.

An exemplary embodiment of the present application provides an organic light-emitting device in which the organic material layer of the organic light-emitting device comprises a light-emitting layer, and the light-emitting layer comprises the heterocyclic compound.

An exemplary embodiment of the present application provides an organic light-emitting device in which the organic material layer of the organic light-emitting device comprises a light-emitting layer, the light-emitting layer comprises a host material, and the host material comprises the heterocyclic compound.

In the organic light-emitting device of the present invention, the organic material layer may comprise an electron injection layer or an electron transport layer, and the electron injection layer or the electron transport layer may comprise the heterocyclic compound.

In another organic light-emitting device, the organic material layer may comprise a hole blocking layer, and the hole blocking layer may comprise the heterocyclic compound.

In another organic light-emitting device, the organic material layer may comprise an electron blocking layer, and the electron blocking layer may comprise the heterocyclic compound.

In another organic light-emitting device, the organic material layer may comprise a hole transport layer, a light-emitting layer, or an electron blocking layer, and the hole transport layer, the light-emitting layer, or the electron blocking layer may comprise the heterocyclic compound.

In another organic light-emitting device, the organic material layer may comprise a hole transport layer or a hole transport auxiliary layer, and the hole transport layer or the hole transport auxiliary layer may comprise the heterocyclic compound.

In the organic light-emitting device of the present application, materials having a relatively large work function may be used as an anode material, and a transparent conductive oxide, a metal, a conductive polymer, and the like may be used. Specific examples of the anode material include metals such as vanadium, chromium, copper, zinc, and gold, or alloys thereof; metal oxides such as zinc oxide, indium oxide, indium tin oxide (ITO), and indium zinc oxide (IZO); a combination of a metal and an oxide, such as ZnO:Al or SnO₂:Sb; conductive polymers such as poly(3-methylthiophene), poly[3,4-(ethylene-1,2-dioxy)thiophene] (PEDOT), polypyrrole, polyaniline, and the like, but are not limited thereto.

Materials having a relatively low work function may be used as a cathode material, and a metal, a metal oxide, a conductive polymer, and the like may be used. Specific examples of the cathode material include metals such as magnesium, calcium, sodium, potassium, titanium, indium, yttrium, lithium, gadolinium, aluminum, silver, tin, and lead, or alloys thereof; a multilayer structure material such as LiF/Al or LiO₂/Al, and the like, but are not limited thereto.

A known hole injection material may be used as the hole injection material, for example, a phthalocyanine compound such as copper phthalocyanine disclosed in U.S. Pat. No. 4,356,429, or starburst-type amine derivatives described in the document [Advanced Material, 6, p. 677 (1994)] such as tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl] benzene (m-MTDAPB), a soluble conductive polymer such as polyaniline/dodecylbenzenesulfonic acid, poly(3,4-ethylenedioxythiophene)/poly(4-styrenesulfonate), polyaniline/camphor sulfonic acid, or polyaniline/poly(4-styrene-sulfonate), and the like may be used.

A pyrazoline derivative, an arylamine-based derivative, a stilbene derivative, a triphenyldiamine derivative, and the like may be used as a hole transport material, and a low-molecular weight or high-molecular weight material may also be used.

As an electron transport material, an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like may be used, and not only a low-molecular weight material but also a high-molecular weight material may be used.

For example, LiF is typically used in the art as an electron injection material, but the present application is not limited thereto.

A red, green, or blue light-emitting material may be used as a light-emitting material, and if necessary, two or more light-emitting materials may be mixed and used. In this case, two or more light-emitting materials may be deposited and used as individual sources, or may be premixed and deposited and used as a single source. In addition, a fluorescent material may be used as the light-emitting material, but a phosphorescent material may also be used. As the light-emitting material, a material emitting light by combining holes and electrons respectively injected from an anode and a cathode by itself may be used alone, but materials in which a host material and a dopant material participate in light emission together may also be used.

When a host of the light-emitting material is mixed and used, hosts of the same series may be mixed and used, or hosts of different series may be mixed and used. For example, two or more kinds of materials among an n-type host material and a p-type host material may be selected and used as the host material of the light-emitting layer.

The organic light-emitting device according to an exemplary embodiment of the present application may be a top-emission type, a bottom-emission type, or a dual-emission type depending on the material to be used.

The heterocyclic compound according to an exemplary embodiment of the present application may operate in an organic electronic device including an organic solar cell, an organic photoreceptor, an organic transistor, and the like on a principle similar to that applied to the organic light-emitting device.

The organic light-emitting device of the present invention may further comprise one or more layers selected from the group consisting of a light-emitting layer, a hole injection layer, a hole transport layer, a hole transport auxiliary layer, an electron blocking layer, a hole blocking layer, an electron transport layer, and an electron injection layer.

The organic light-emitting device of the present invention may further comprise one or more layers selected from the group consisting of a light-emitting layer, a hole injection layer, a hole transport layer, an electron blocking layer, a hole blocking layer, an electron transport layer, and an electron injection layer.

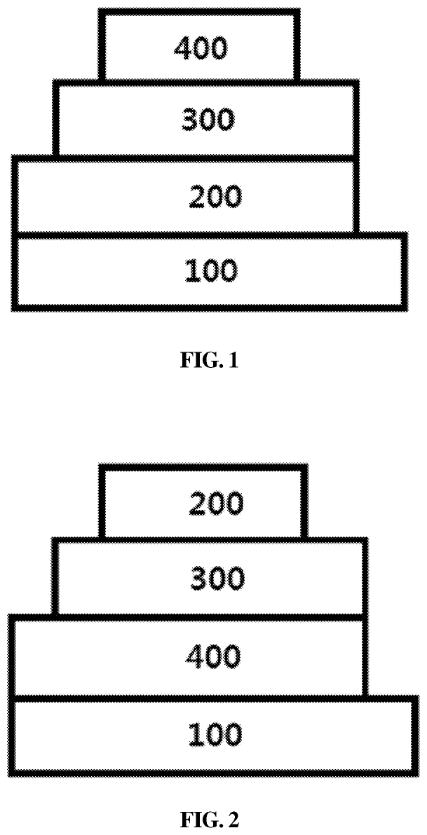

FIGS. 1 to 3 illustrate a stacking order of an electrode and an organic material layer of the organic light-emitting device according to an exemplary embodiment of the present application. However, the scope of the present application is not intended to be limited by these drawings, and a structure of an organic light-emitting device known in the art may also be applied to the present application.

According to FIG. 1, an organic light-emitting device in which an anode 200, an organic material layer 300, and a cathode 400 are sequentially stacked on a substrate 100 is illustrated. However, it is not limited only to such a structure, and as shown in FIG. 2, an organic light-emitting device in which a cathode, an organic material layer, and an anode are sequentially stacked on a substrate may also be implemented.

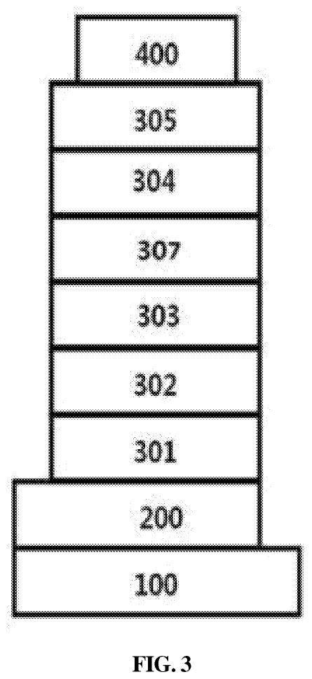

FIG. 3 illustrates a case where the organic material layer is a multilayer.

The organic light-emitting device according to FIG. 3 comprises a hole injection layer 301, a hole transport layer 302, a light-emitting layer 303, a hole blocking layer 307, an electron transport layer 304, and an electron injection layer 305.

However, the scope of the present application is not limited by such a stacked structure, and if necessary, remaining layers except for the light-emitting layer may be omitted, and another necessary functional layer may be further added.

The organic material layer comprising Chemical Formula 1 may additionally comprise another material as necessary.

In an exemplary embodiment of the present application, there is provided an organic light-emitting device wherein the host material further comprises a heterocyclic compound represented by the following Chemical Formula 3 or Chemical Formula 4.

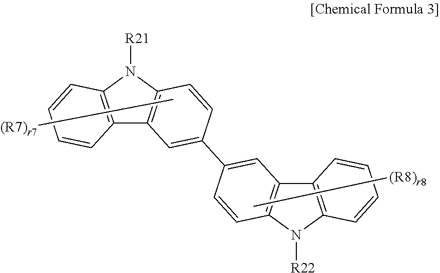

In Chemical Formula 3,
R21 and R22 are the same as or different from each other, and are each independently a substituted or unsubstituted C6 to C60 aryl group; or a substituted or unsubstituted C2 to C60 heteroaryl group;
R7 and R8 are the same as or different from each other, and are each independently hydrogen, deuterium, a halogen group, a cyano group, a substituted or unsubstituted C1 to C60 alkyl group; a substituted or unsubstituted C6 to C60 aryl group; or a substituted or unsubstituted C2 to C60 heteroaryl group; and
r7 and r8 are each an integer from 0 to 7, and when r7 is 2 or greater, R7 are the same as or different from each other, and when r8 is 2 or greater, R8 are the same as or different from each other; and

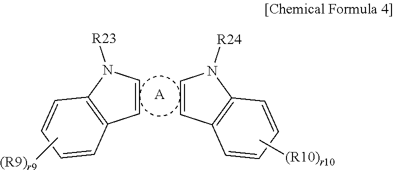

In Chemical Formula 4,
A is a substituted or unsubstituted C6 to C60 aromatic hydrocarbon ring;
R23 and R24 are the same as or different from each other, and are each independently a substituted or unsubstituted C6 to C60 aryl group; or a substituted or unsubstituted C2 to C60 heteroaryl group;
R9 and R10 are the same as or different from each other, and are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group; and
r9 and r10 are each an integer from 0 to 4, and when r9 is 2 or greater, R9 are the same as or different from each other, and when r10 is 2 or greater, R10 are the same as or different from each other.

In an exemplary embodiment of the present application, R21 and R22 are the same as or different from each other, and may each independently be a substituted or unsubstituted C6 to C40 aryl group; or a substituted or unsubstituted C2 to C40 heteroaryl group.

In another exemplary embodiment, R21 and R22 are the same as or different from each other, and may each independently be a substituted or unsubstituted C6 to C20 aryl group; or a substituted or unsubstituted C2 to C20 heteroaryl group.

In another exemplary embodiment, R21 and R22 are the same as or different from each other, and may each independently be a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted phenanthrene group; a substituted or unsubstituted triphenylene group; a substituted or unsubstituted dibenzofuran group; or a substituted or unsubstituted dibenzothiophene group.

In an exemplary embodiment of the present application, R7 and R8 are the same as or different from each other, and may each independently be hydrogen; deuterium; a substituted or unsubstituted C1 to C20 alkyl group; a substituted or unsubstituted C6 to C20 aryl group; or a substituted or unsubstituted C2 to C20 heteroaryl group.

In another exemplary embodiment, R7 and R8 are the same as or different from each other, and may each independently be hydrogen; deuterium; a substituted or unsubstituted methyl group; a substituted or unsubstituted phenyl group; a substituted or unsubstituted biphenyl group; a substituted or unsubstituted naphthyl group; a substituted or unsubstituted dibenzofuran group; or a substituted or unsubstituted dibenzothiophene group.

In another exemplary embodiment, R7 and R8 are the same as or different from each other, and may each independently be hydrogen; deuterium; a substituted or unsubstituted methyl group; or a substituted or unsubstituted phenyl group.

In another exemplary embodiment, R7 and R8 are the same as or different from each other, and may each independently be hydrogen; or deuterium.

In an exemplary embodiment of the present application, A may be a substituted or unsubstituted C6 to C40 aromatic hydrocarbon ring.

In another exemplary embodiment, A may be a substituted or unsubstituted C6 to C20 aromatic hydrocarbon ring.

In another exemplary embodiment, A may be a substituted or unsubstituted benzene ring; or a substituted or unsubstituted naphthalene ring.

In another exemplary embodiment, A may be a substituted or unsubstituted benzene ring.

In another exemplary embodiment, A may be a benzene ring substituted or unsubstituted with deuterium.

In an exemplary embodiment of the present application, R23 and R24 are the same as or different from each other, and may each independently be a substituted or unsubstituted C6 to C40 aryl group; or a substituted or unsubstituted C2 to C40 heteroaryl group.

In another exemplary embodiment, R23 and R24 are the same as or different from each other, and may each independently be a substituted or unsubstituted C6 to C20 aryl group; or a substituted or unsubstituted C2 to C20 heteroaryl group.

In another exemplary embodiment, R23 and R24 are the same as or different from each other, and may each independently be a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted phenanthrene group; a substituted or unsubstituted triphenylene group; a substituted or unsubstituted dibenzofuran group; or a substituted or unsubstituted dibenzothiophene group.

In an exemplary embodiment of the present application, R9 and R10 are the same as or different from each other, and may each independently be hydrogen; deuterium; a substituted or unsubstituted C1 to C20 alkyl group; a substituted or unsubstituted C6 to C20 aryl group; or a substituted or unsubstituted C2 to C20 heteroaryl group.

In another exemplary embodiment, R9 and R10 are the same as or different from each other, and may each independently be hydrogen; deuterium; a substituted or unsubstituted methyl group; a substituted or unsubstituted phenyl group; a substituted or unsubstituted biphenyl group; a substituted or unsubstituted naphthyl group; a substituted or unsubstituted dibenzofuran group; or a substituted or unsubstituted dibenzothiophene group.

In another exemplary embodiment, R9 and R10 are the same as or different from each other, and may each independently be hydrogen; deuterium; a substituted or unsubstituted methyl group; or a substituted or unsubstituted phenyl group.

In another exemplary embodiment, R9 and R10 are the same as or different from each other, and may each independently be hydrogen; or deuterium.

In an exemplary embodiment of the present application, a deuterium content of Chemical Formula 3 may be 0% to 100%.

In another exemplary embodiment, a deuterium content of Chemical Formula 3 may be 0%, or 20% to 100%.

In another exemplary embodiment, a deuterium content of Chemical Formula 3 may be 0%, or 40% to 100%.

In another exemplary embodiment, a deuterium content of Chemical Formula 3 may be 0%, or 60% to 100%.

In another exemplary embodiment, a deuterium content of Chemical Formula 3 may be 0%, or 80% to 100%.

In another exemplary embodiment, a deuterium content of Chemical Formula 3 may be 0%, or 100%.

In an exemplary embodiment of the present application, a deuterium content of Chemical Formula 4 may be 0% to 100%.

In another exemplary embodiment, a deuterium content of Chemical Formula 4 may be 0%, or 20% to 100%.

In another exemplary embodiment, a deuterium content of Chemical Formula 4 may be 0%, or 40% to 100%.

In another exemplary embodiment, a deuterium content of Chemical Formula 4 may be 0%, or 60% to 100%.

In another exemplary embodiment, a deuterium content of Chemical Formula 4 may be 0%, or 80% to 100%.

In another exemplary embodiment, a deuterium content of Chemical Formula 4 may be 0%, or 100%.

An exemplary embodiment of the present application provides an organic light-emitting device in which Chemical Formula 3 is represented by any one of the following compounds.

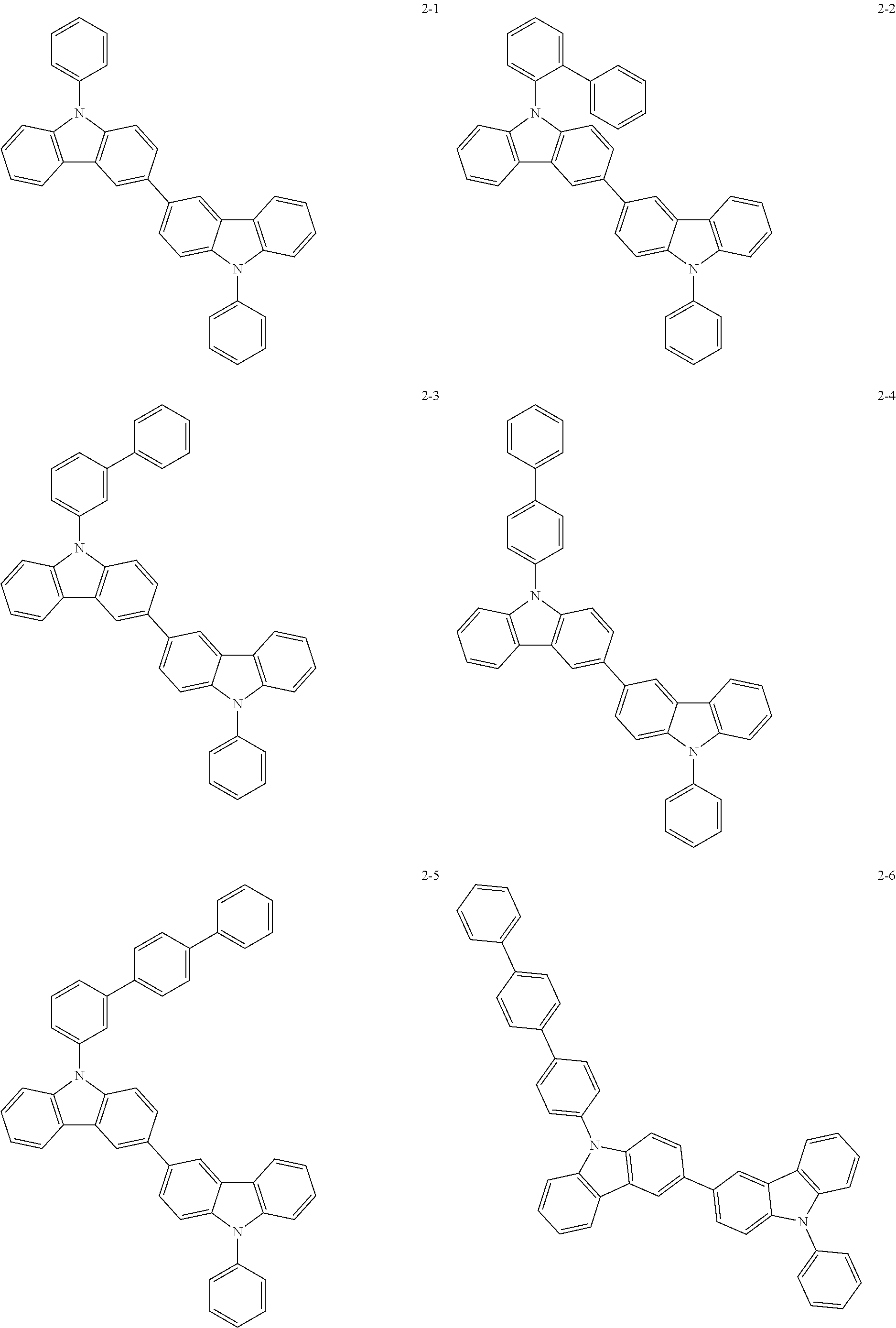 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

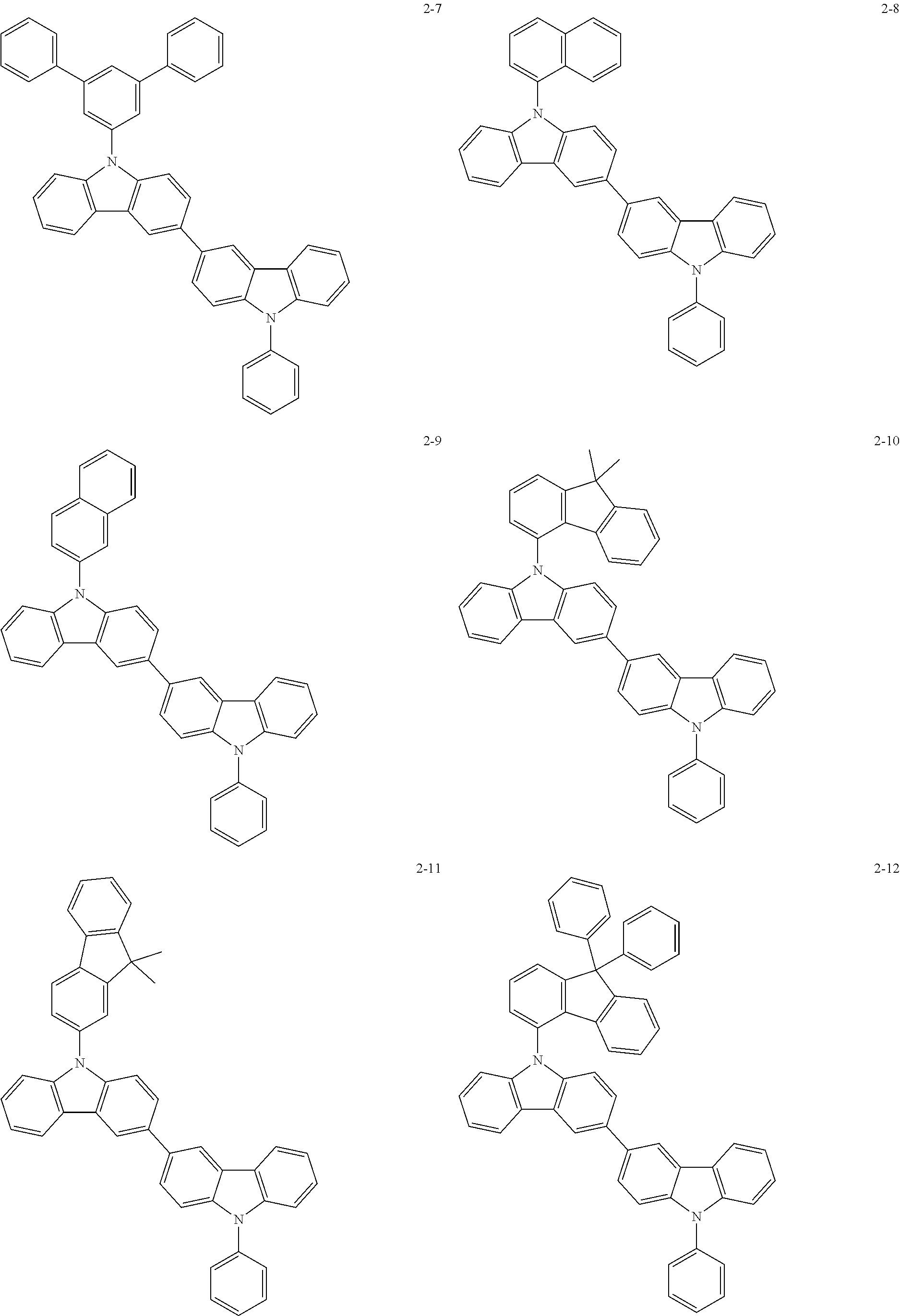 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

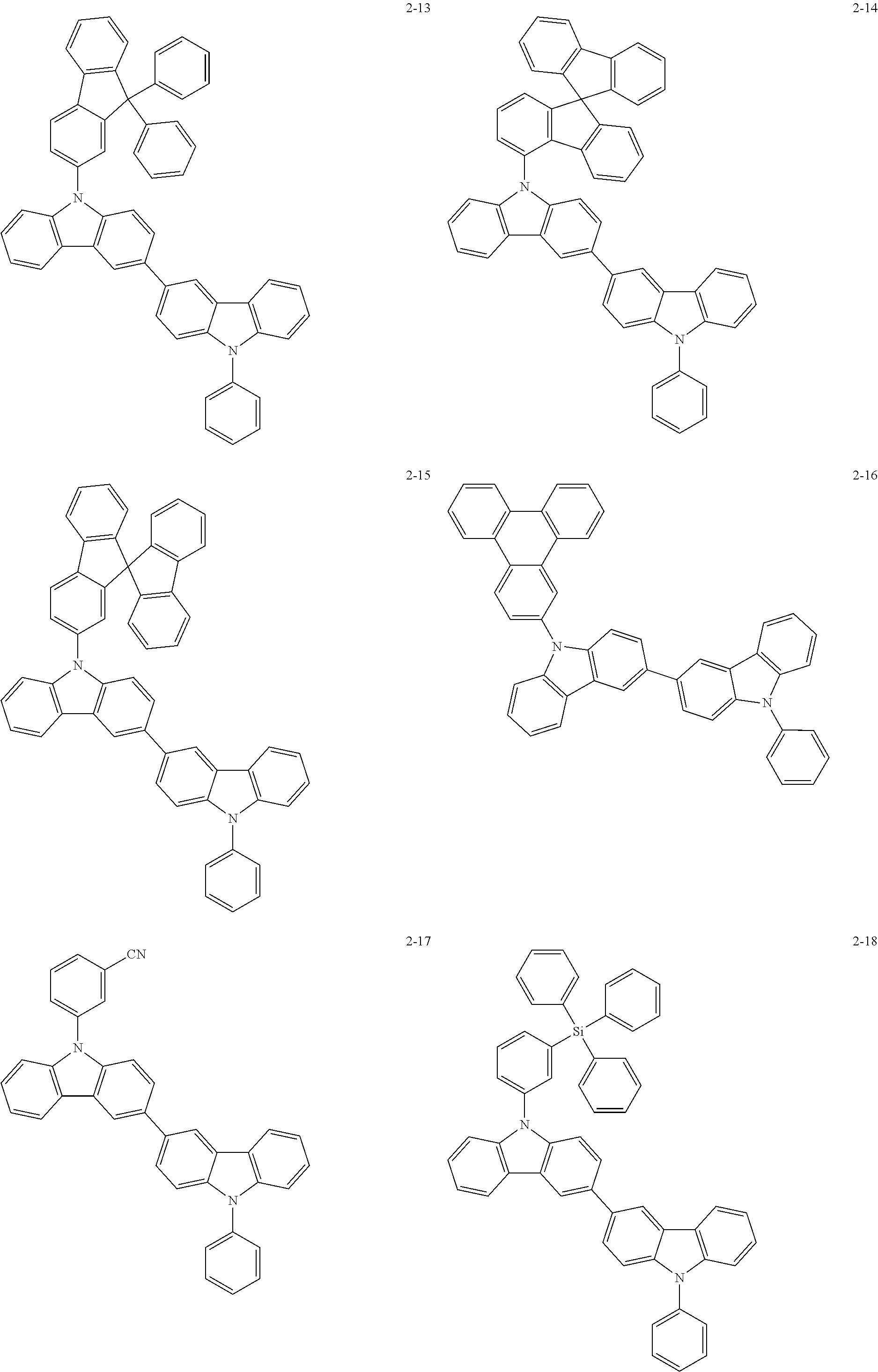

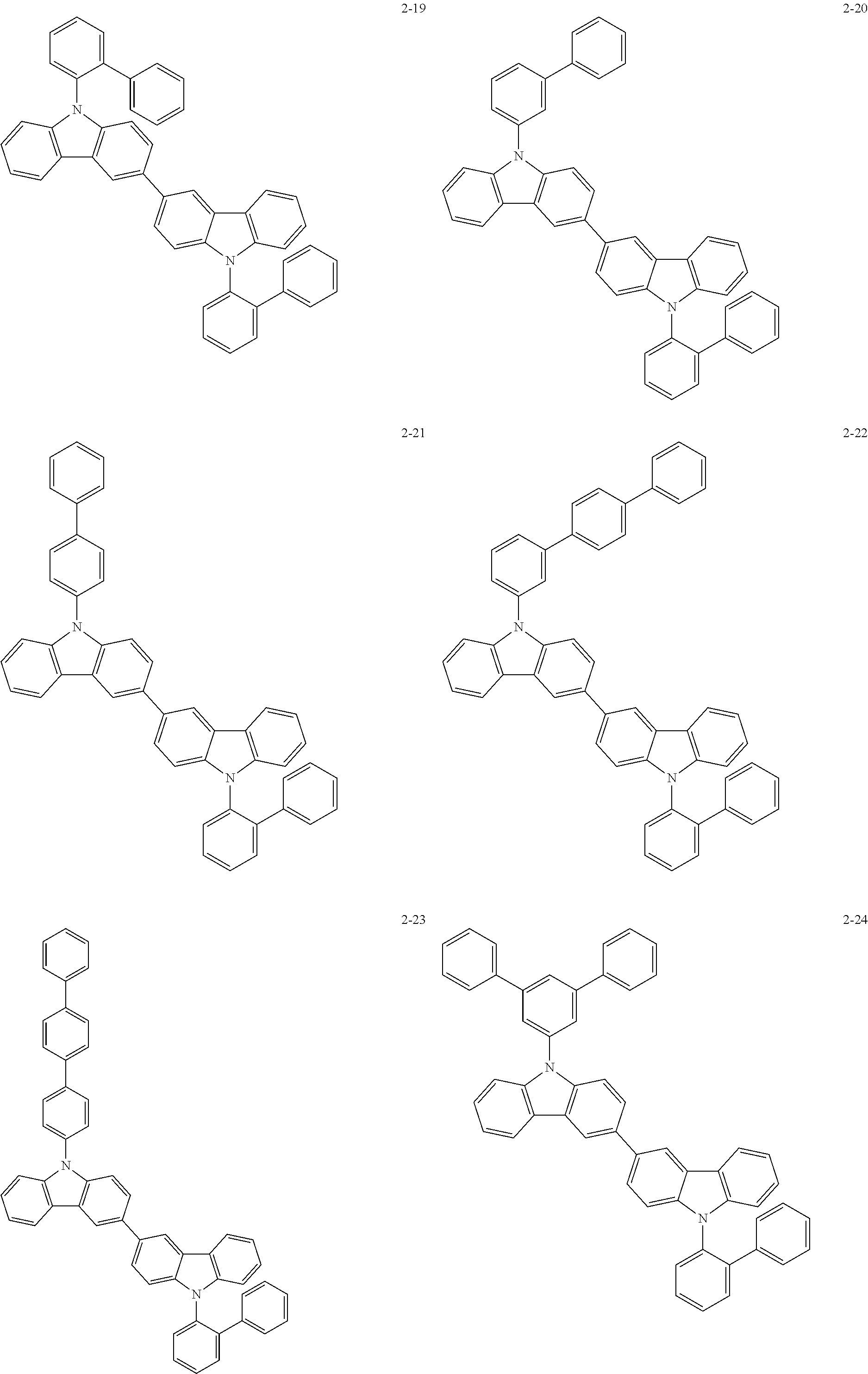

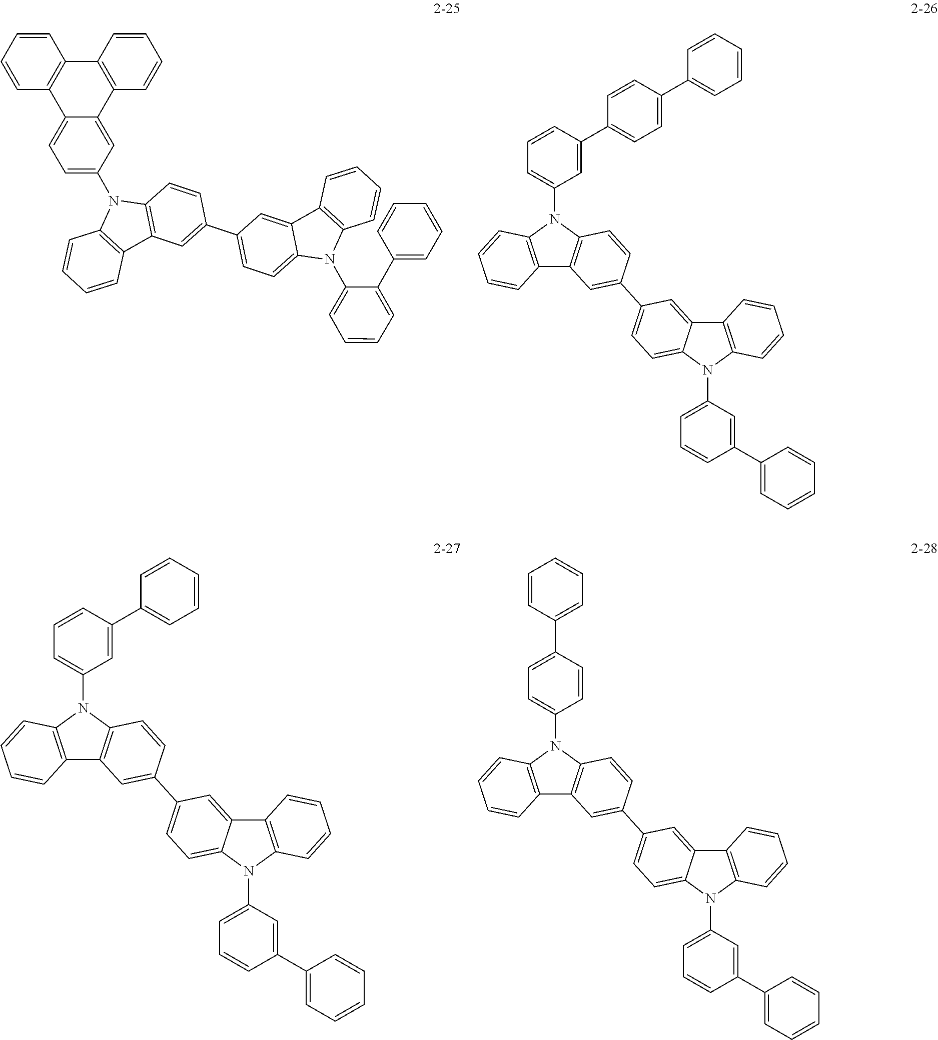

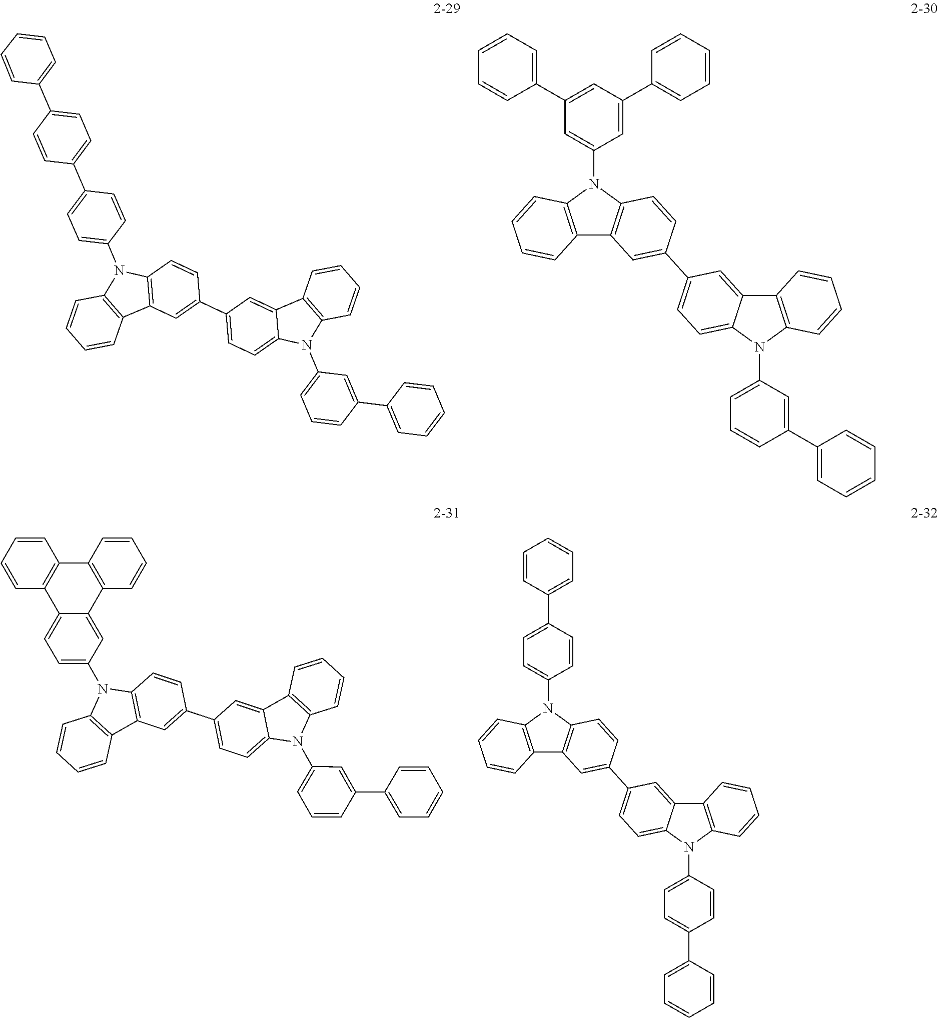 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

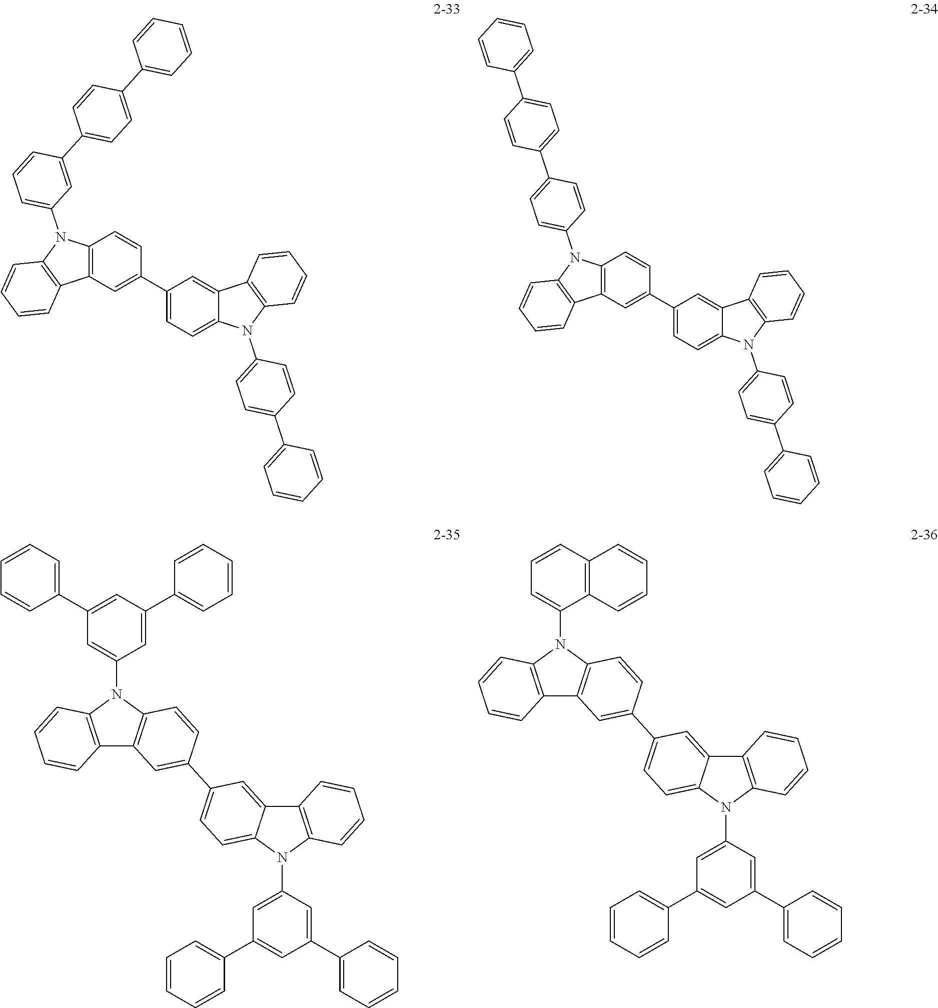 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

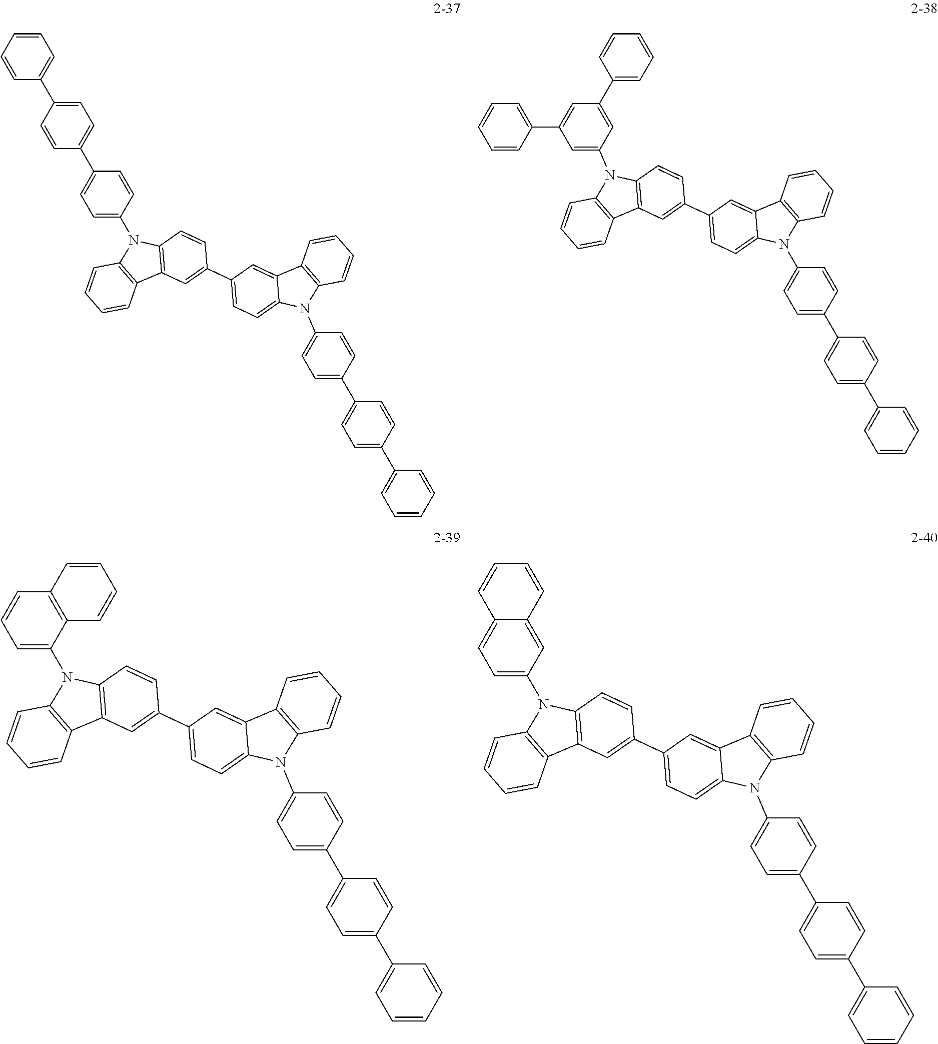 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

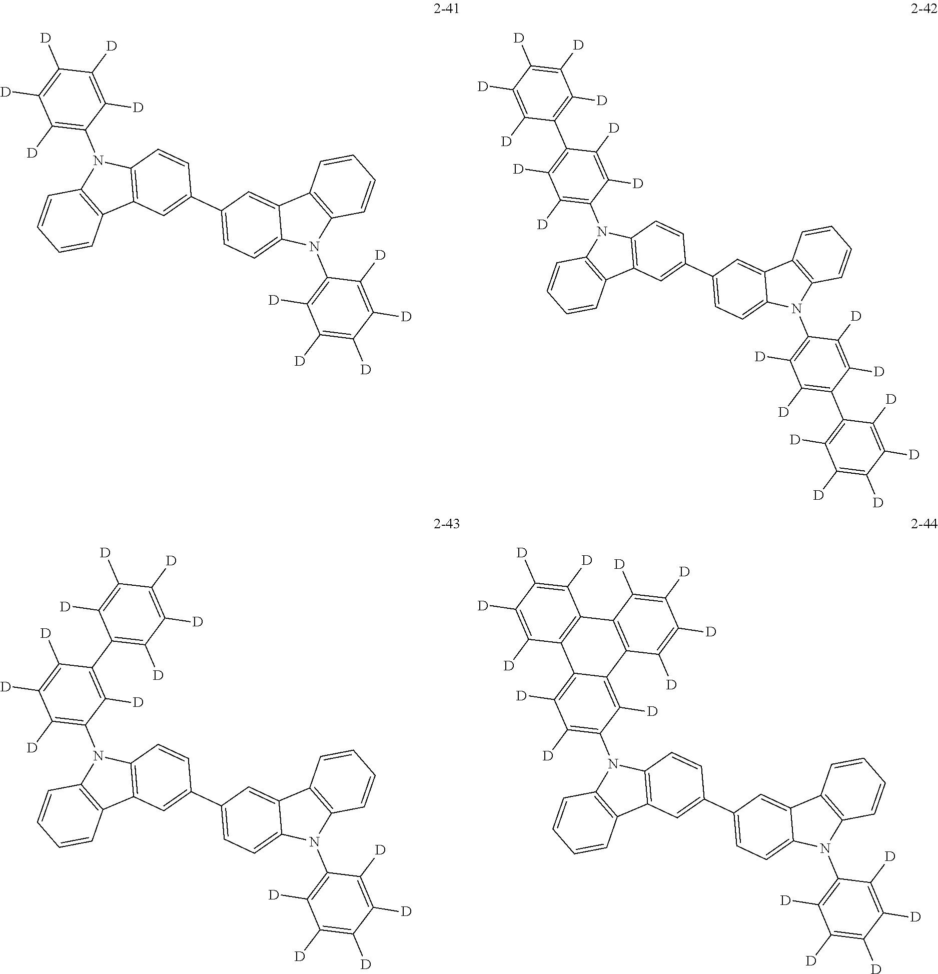

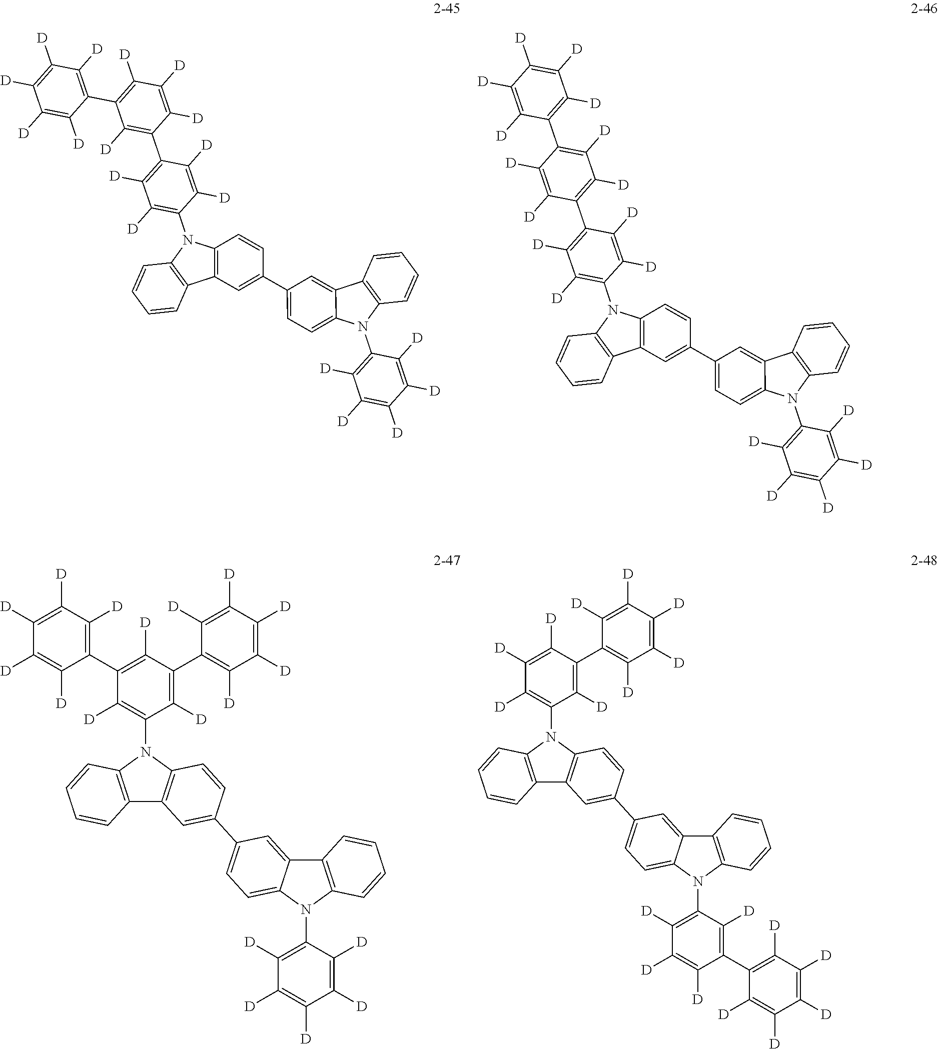

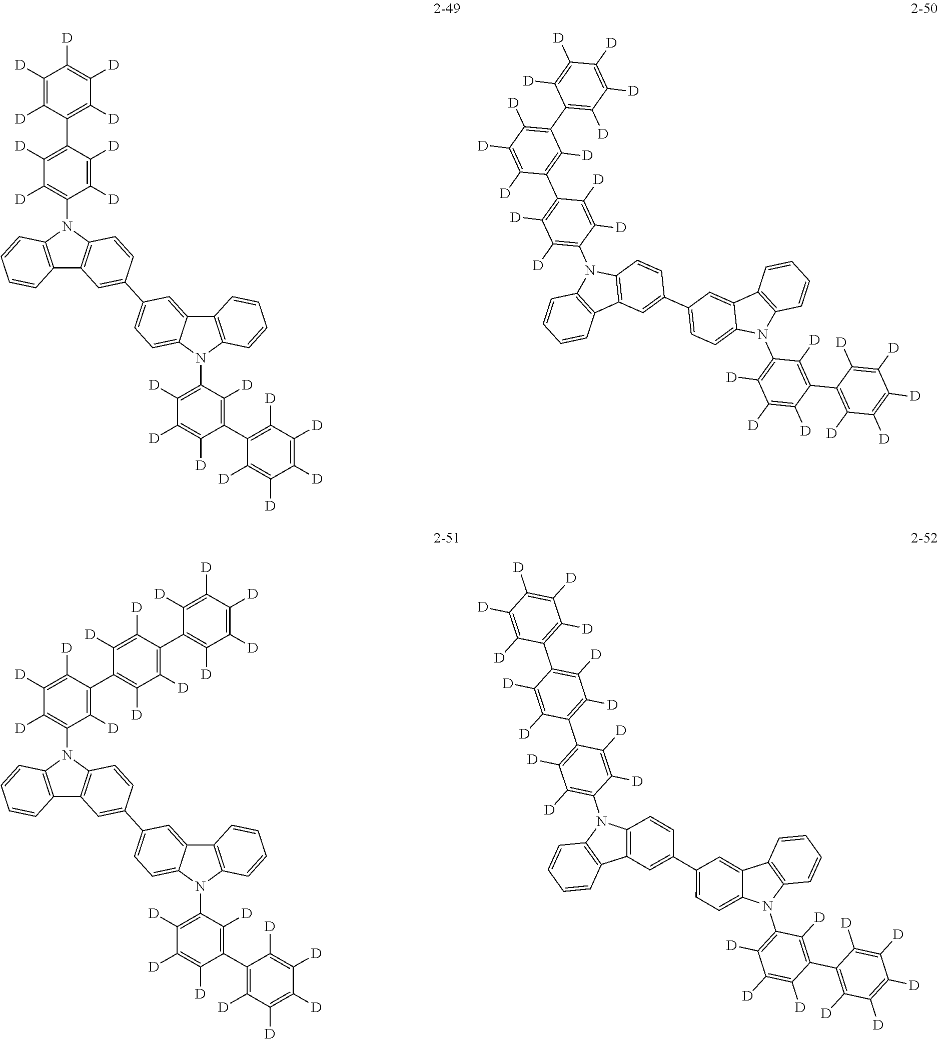

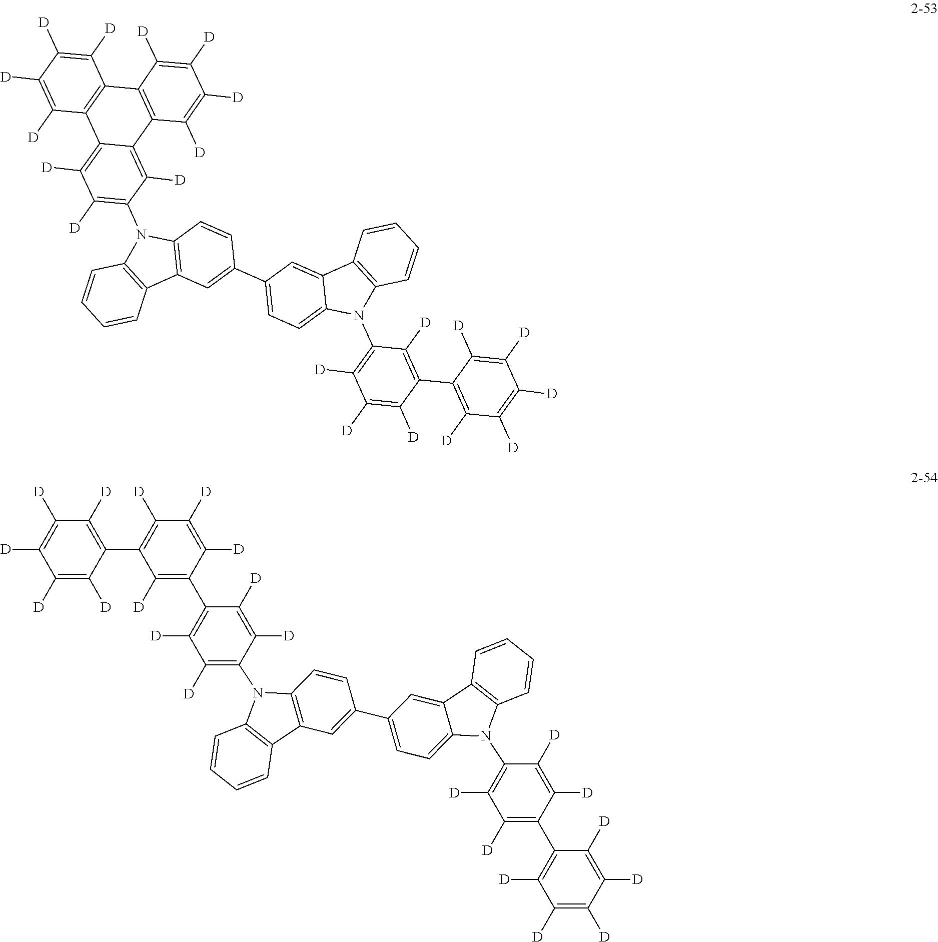 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c(-c7c([2H])c([2H])c([2H])c([2H])c7[2H])c6[2H])c([2H])c5[2H])ccc43)c([2H])c2[2H])c([2H])c1[2H]]

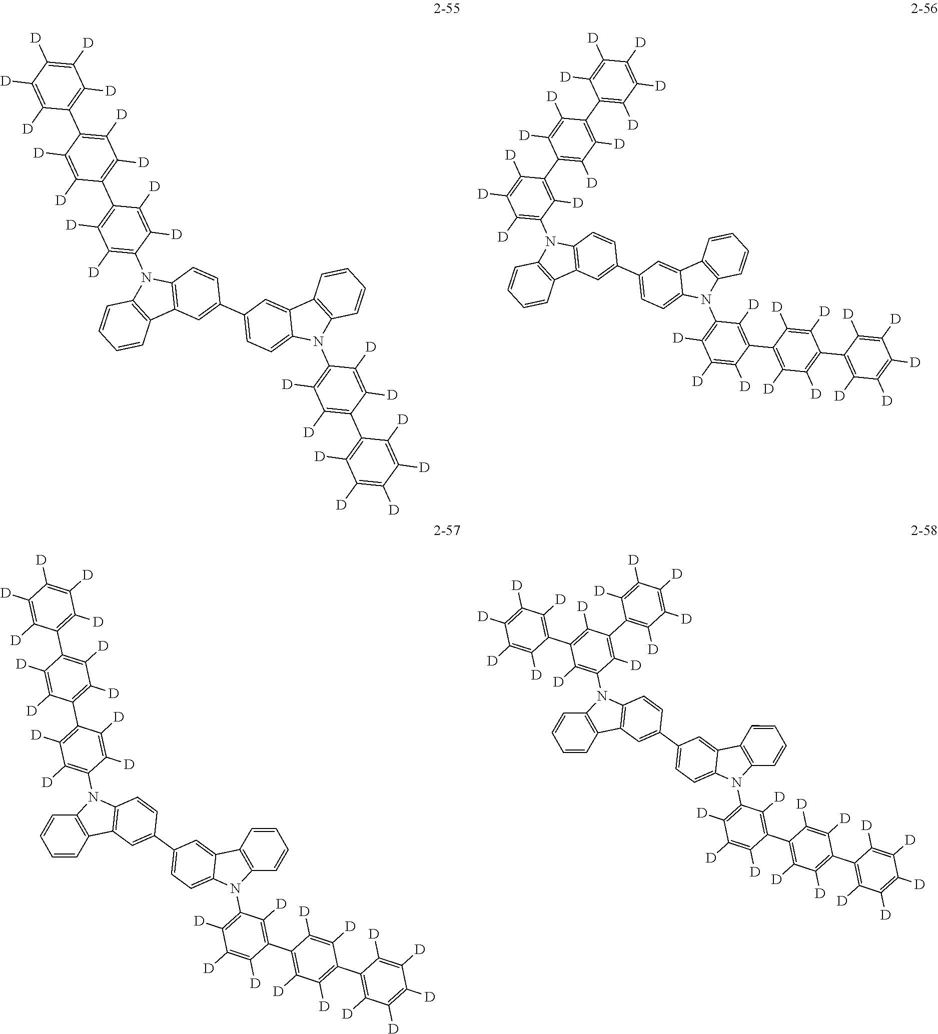

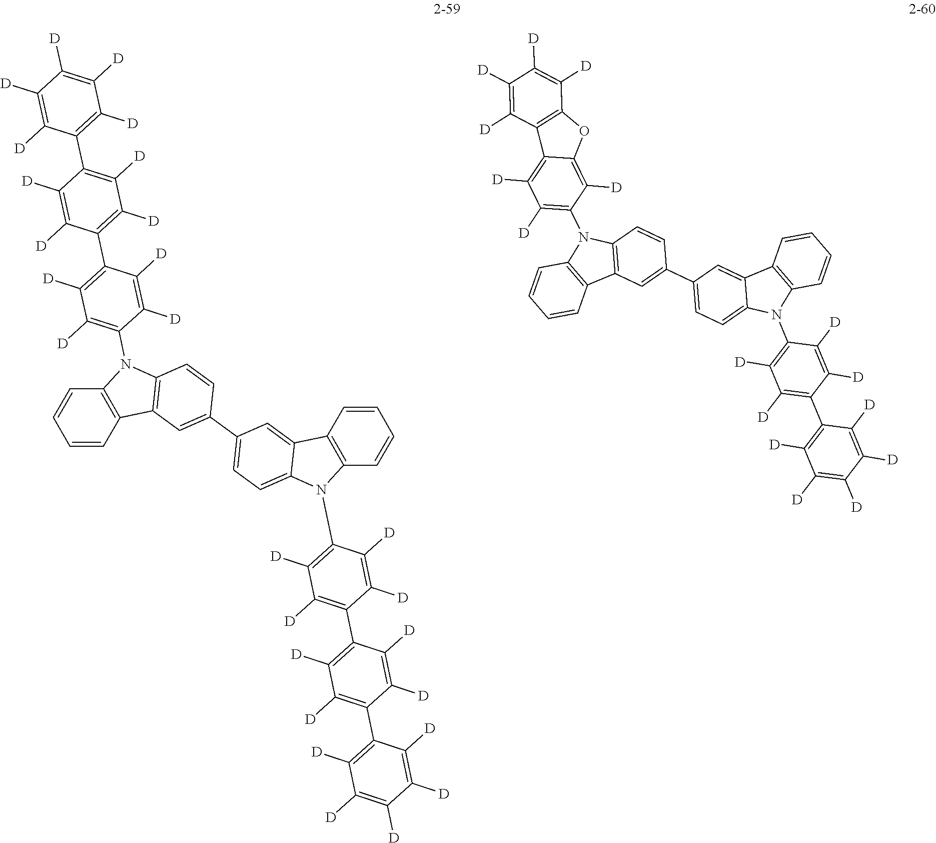 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c(-c8c([2H])c([2H])c([2H])c([2H])c8[2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H]]

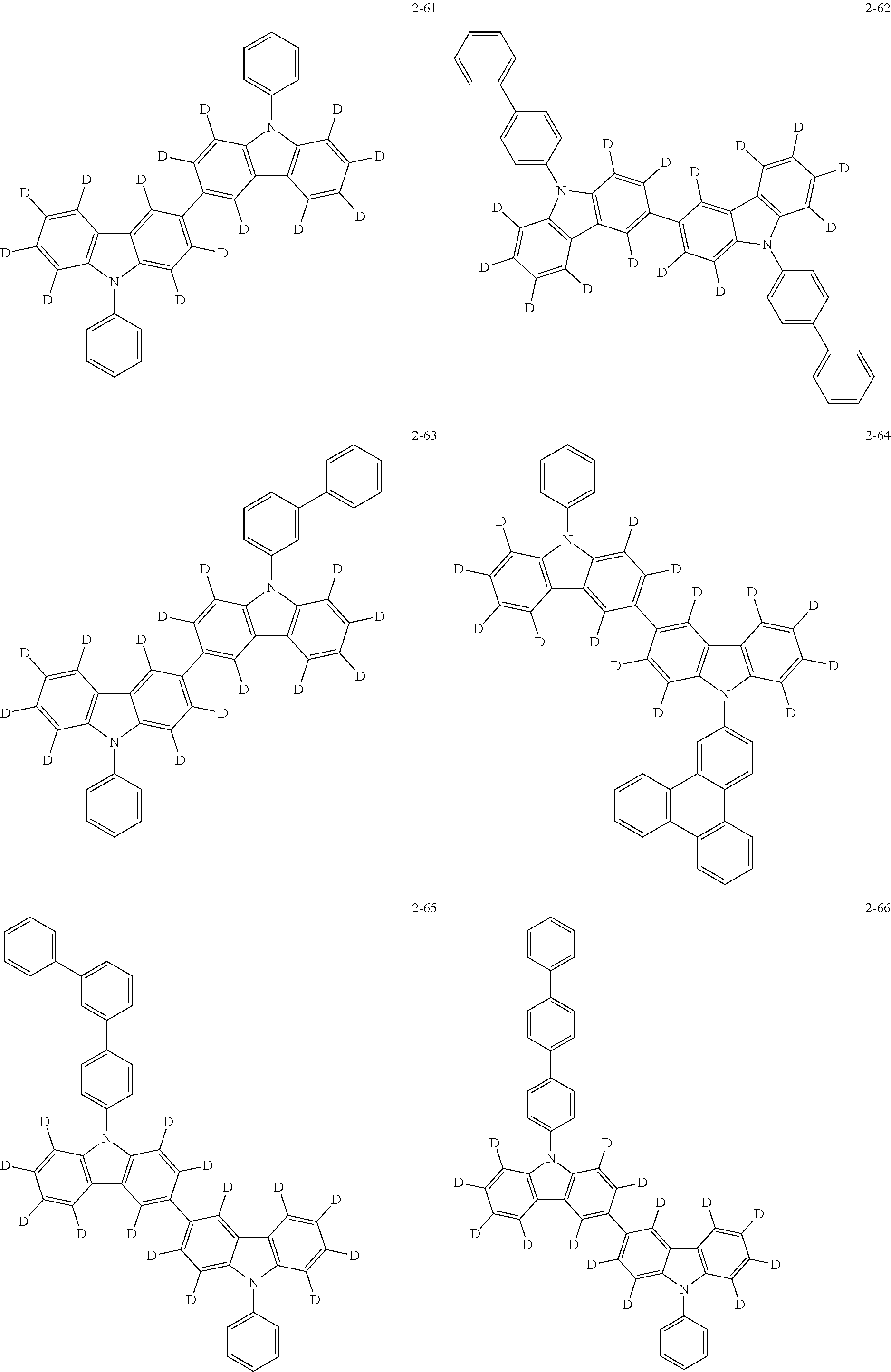

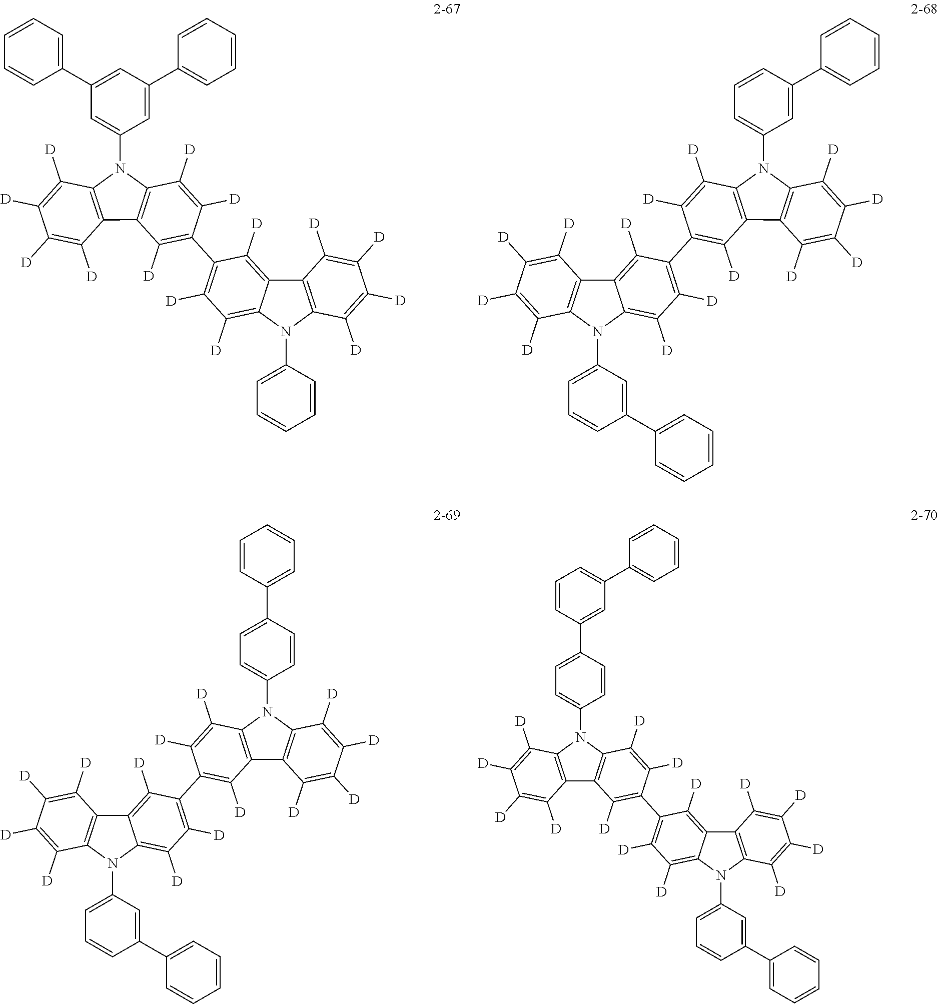

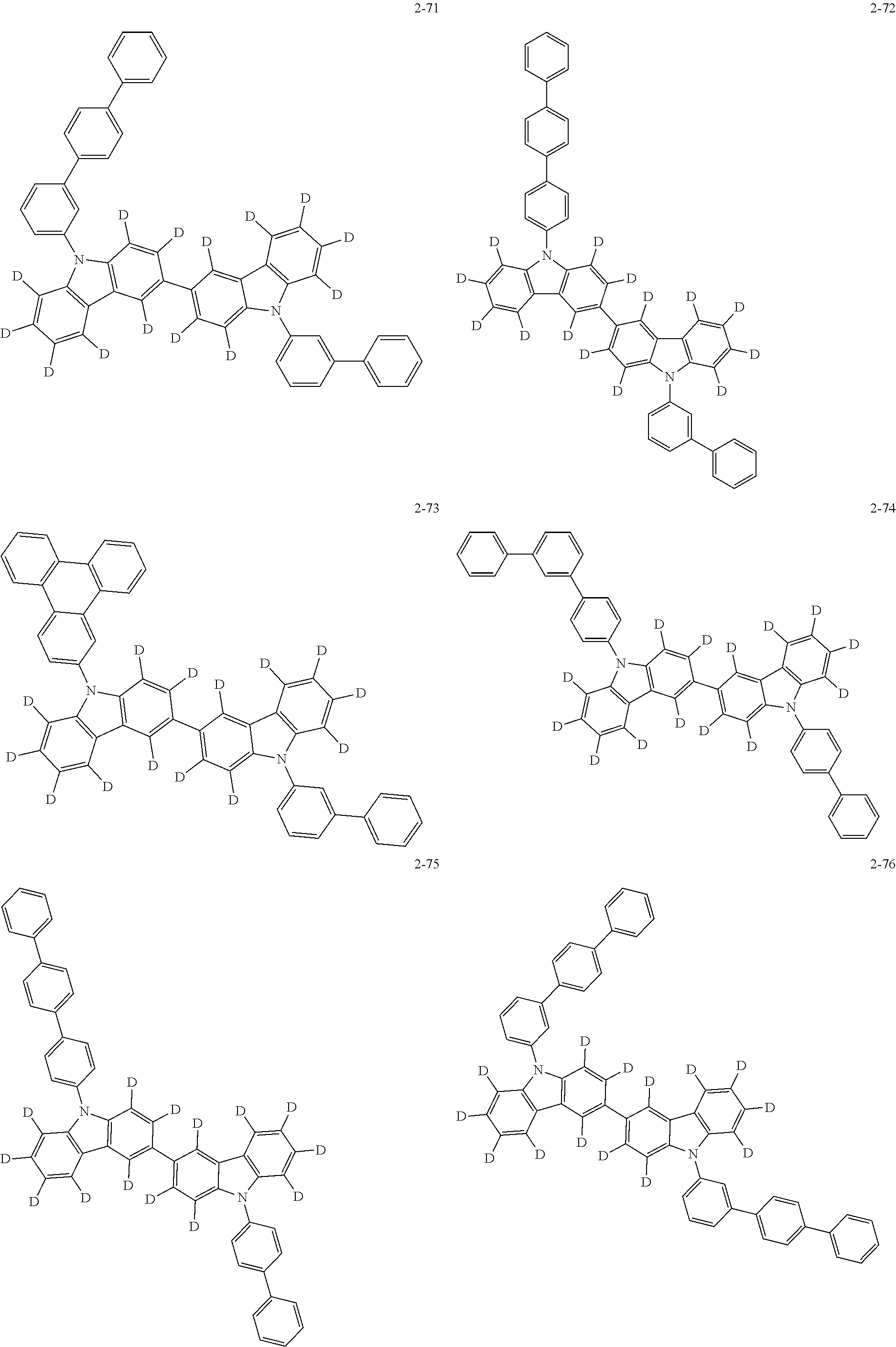

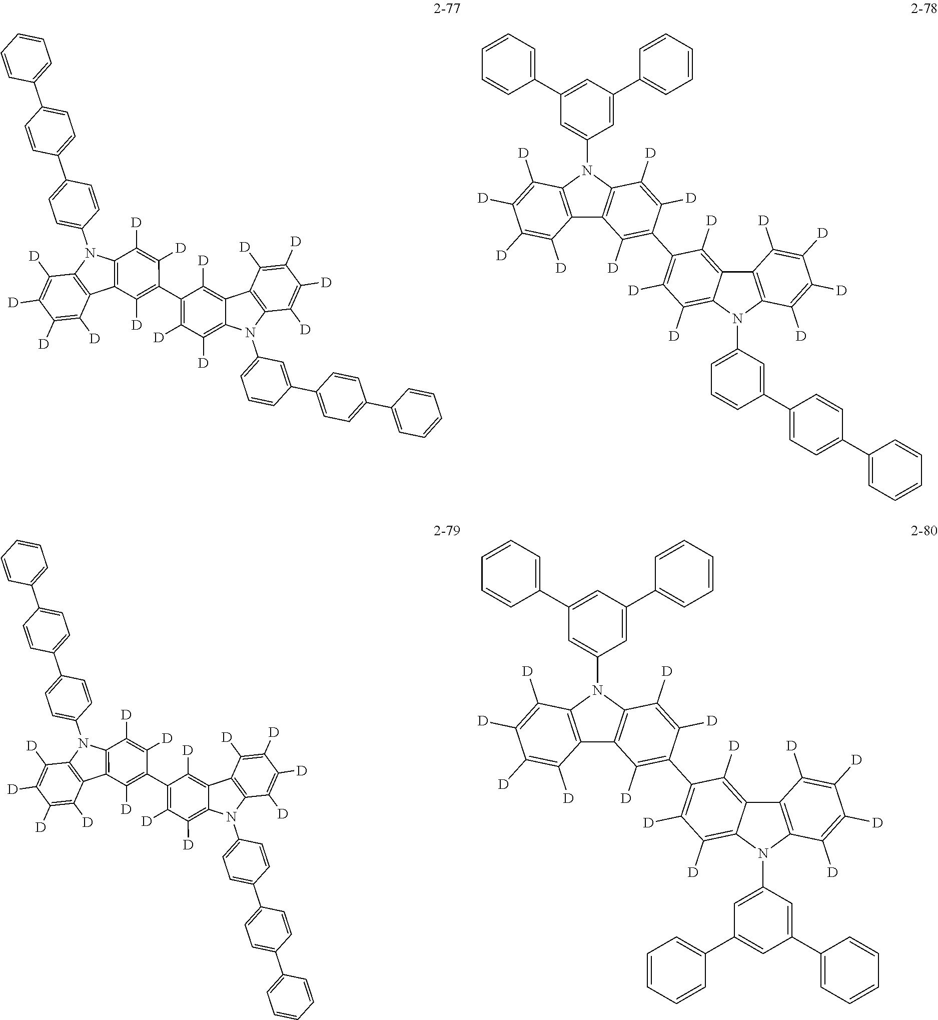

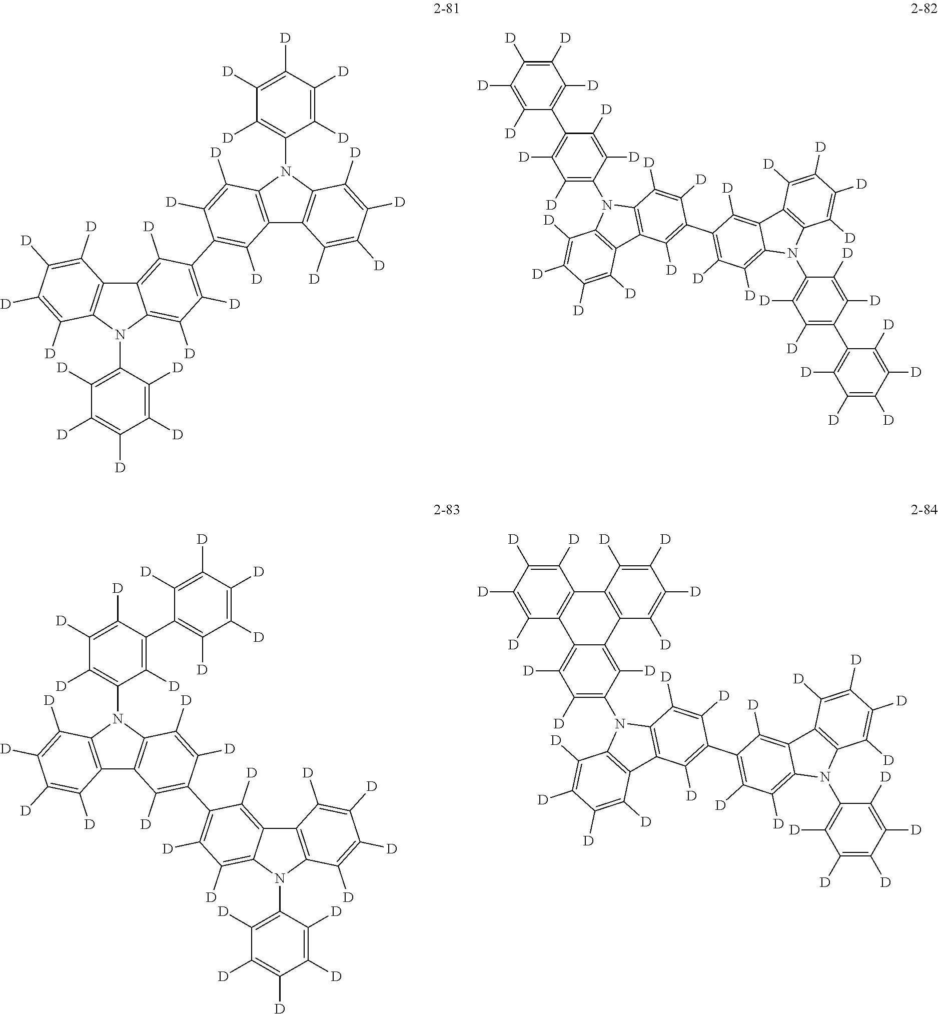

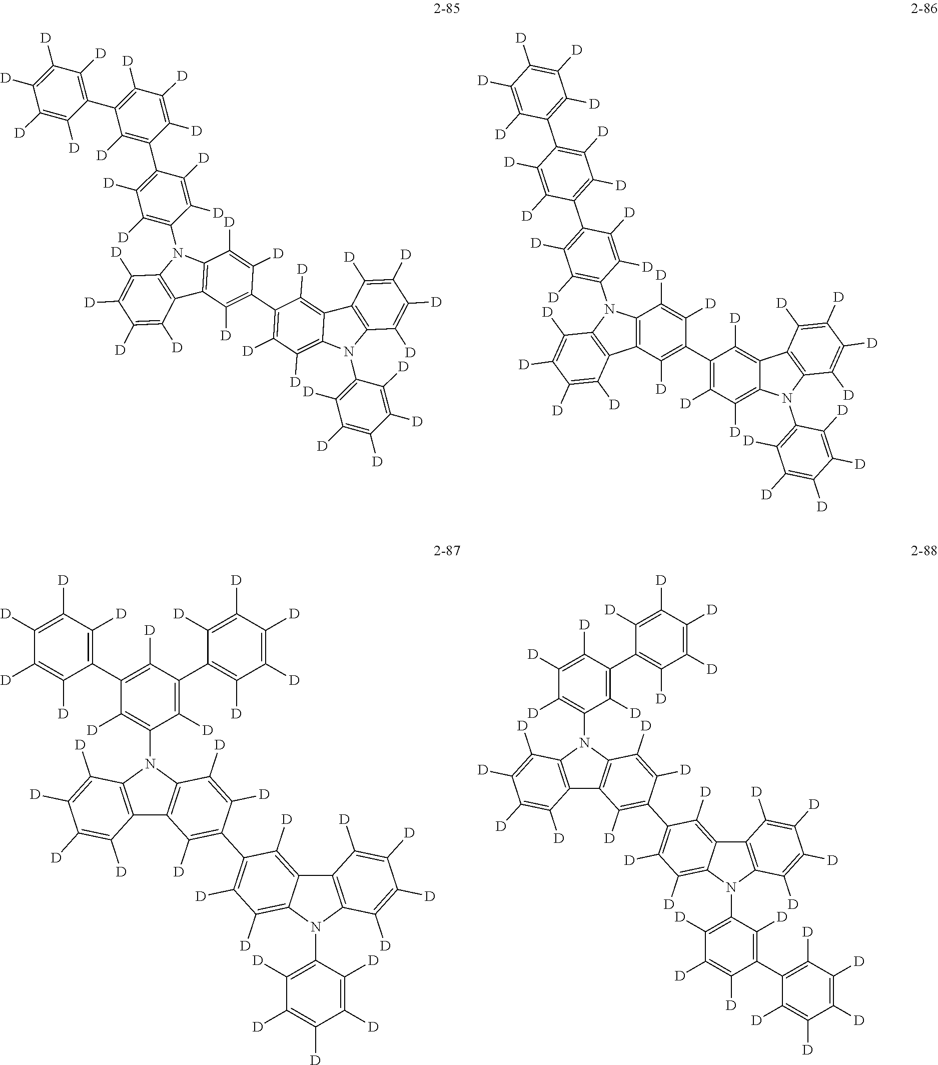

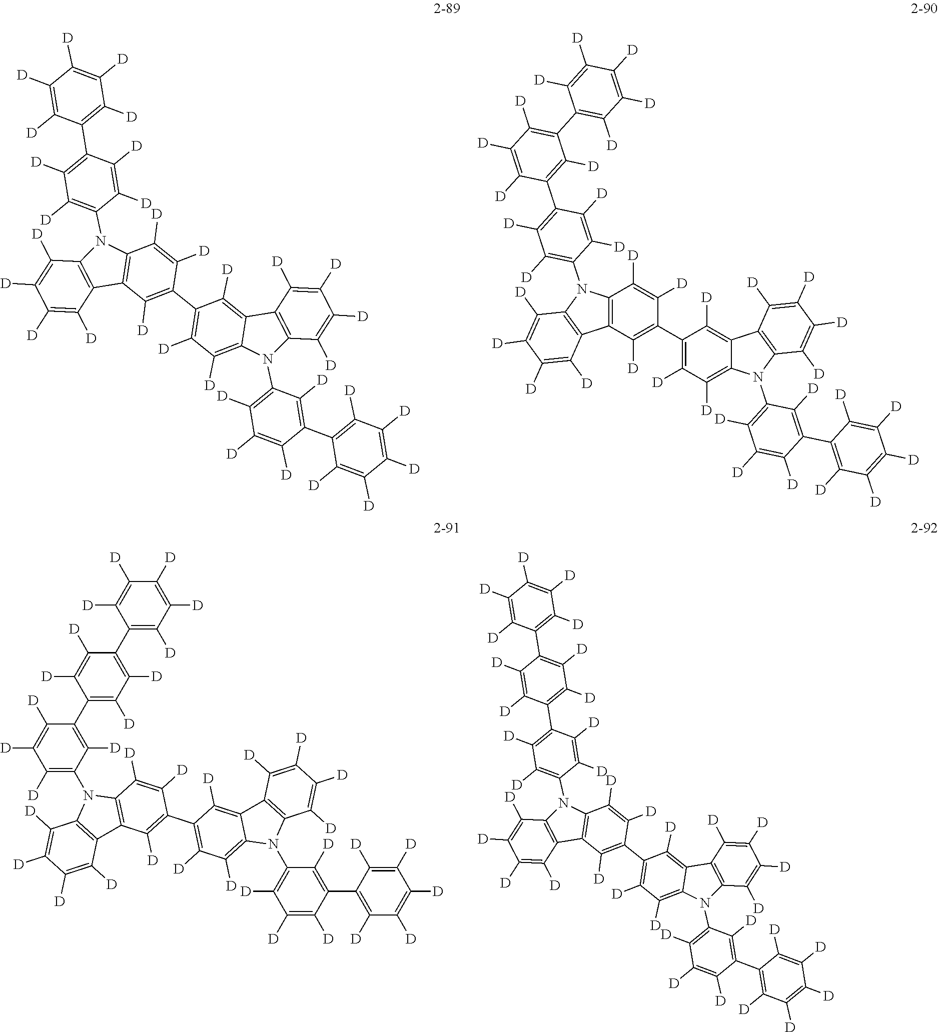

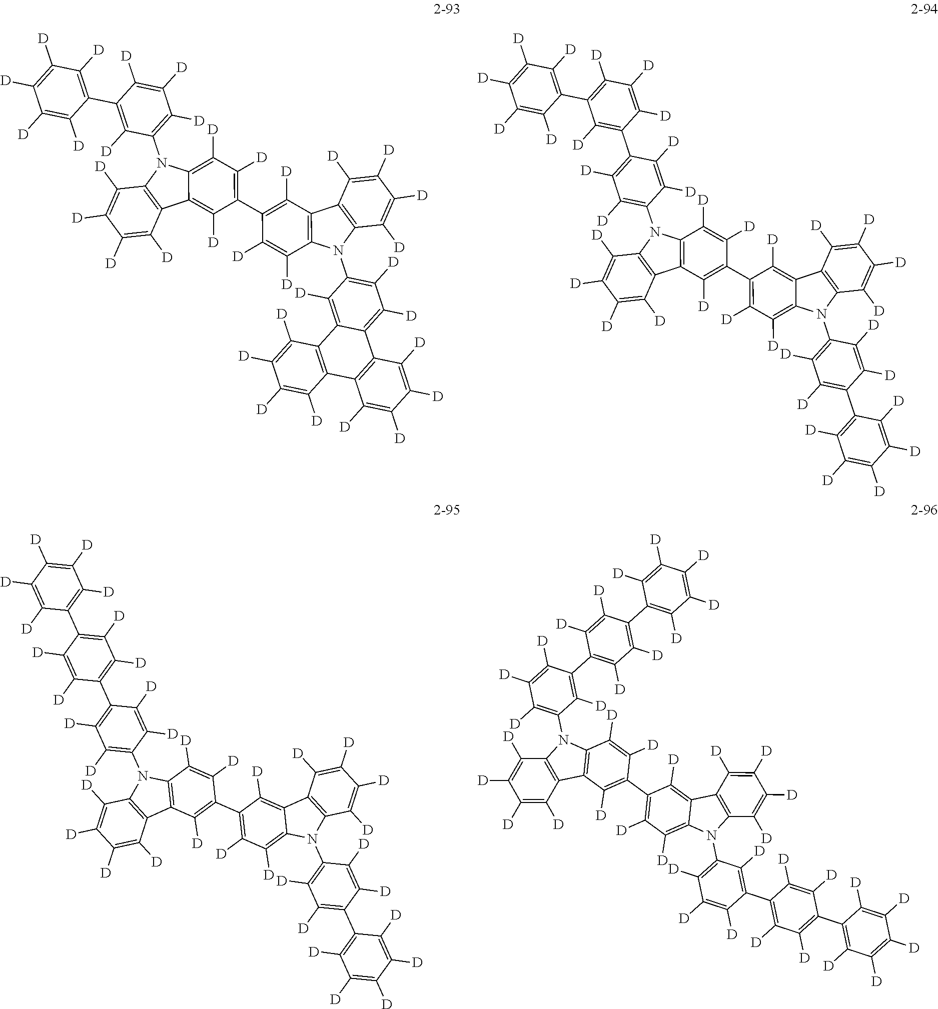

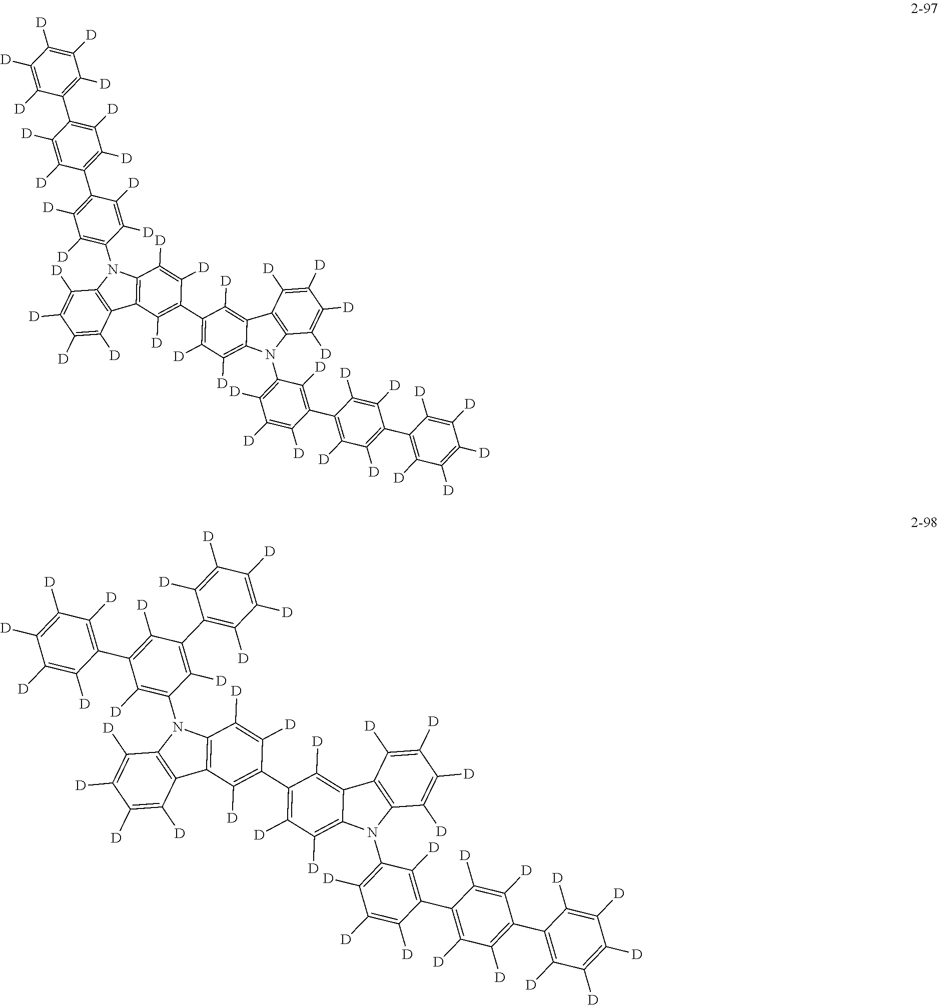

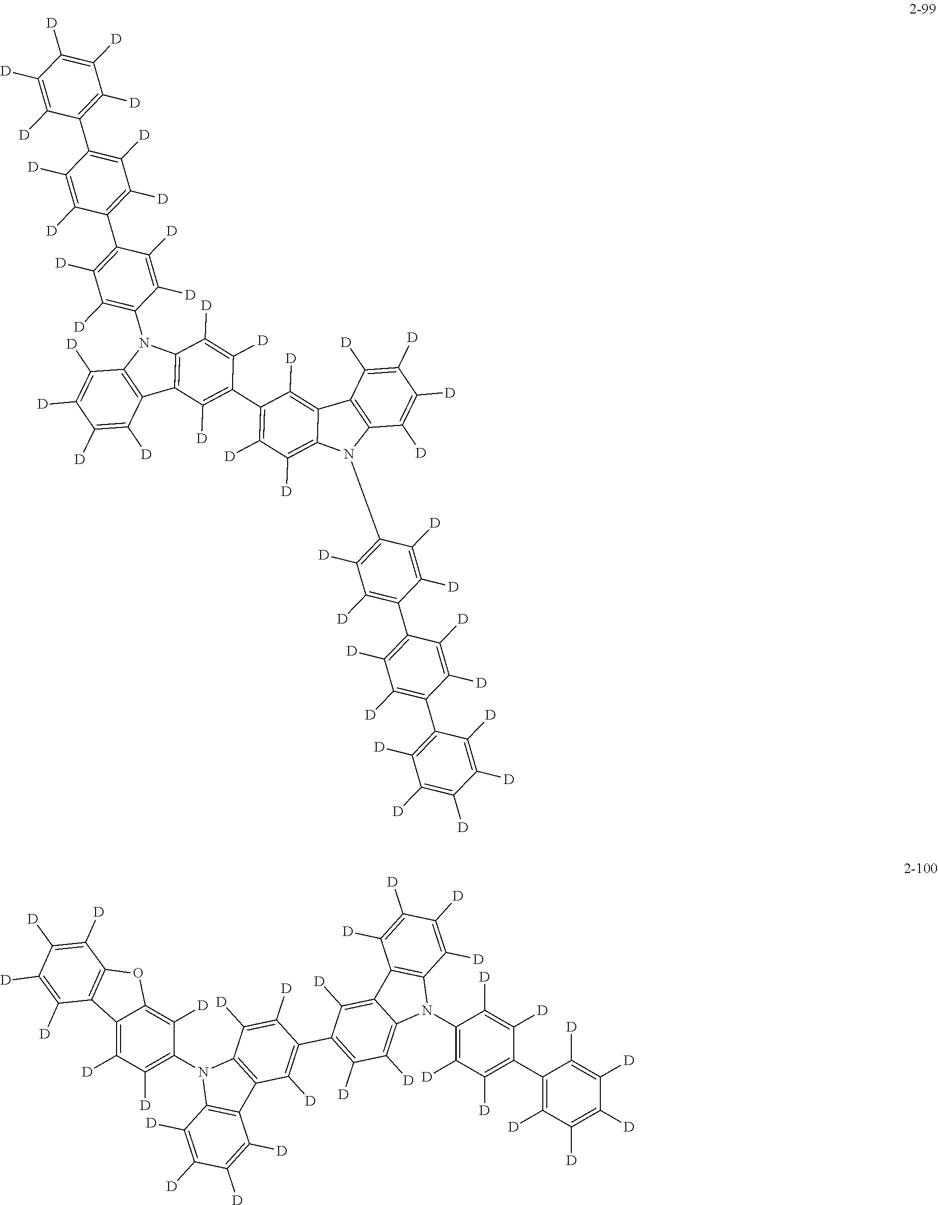

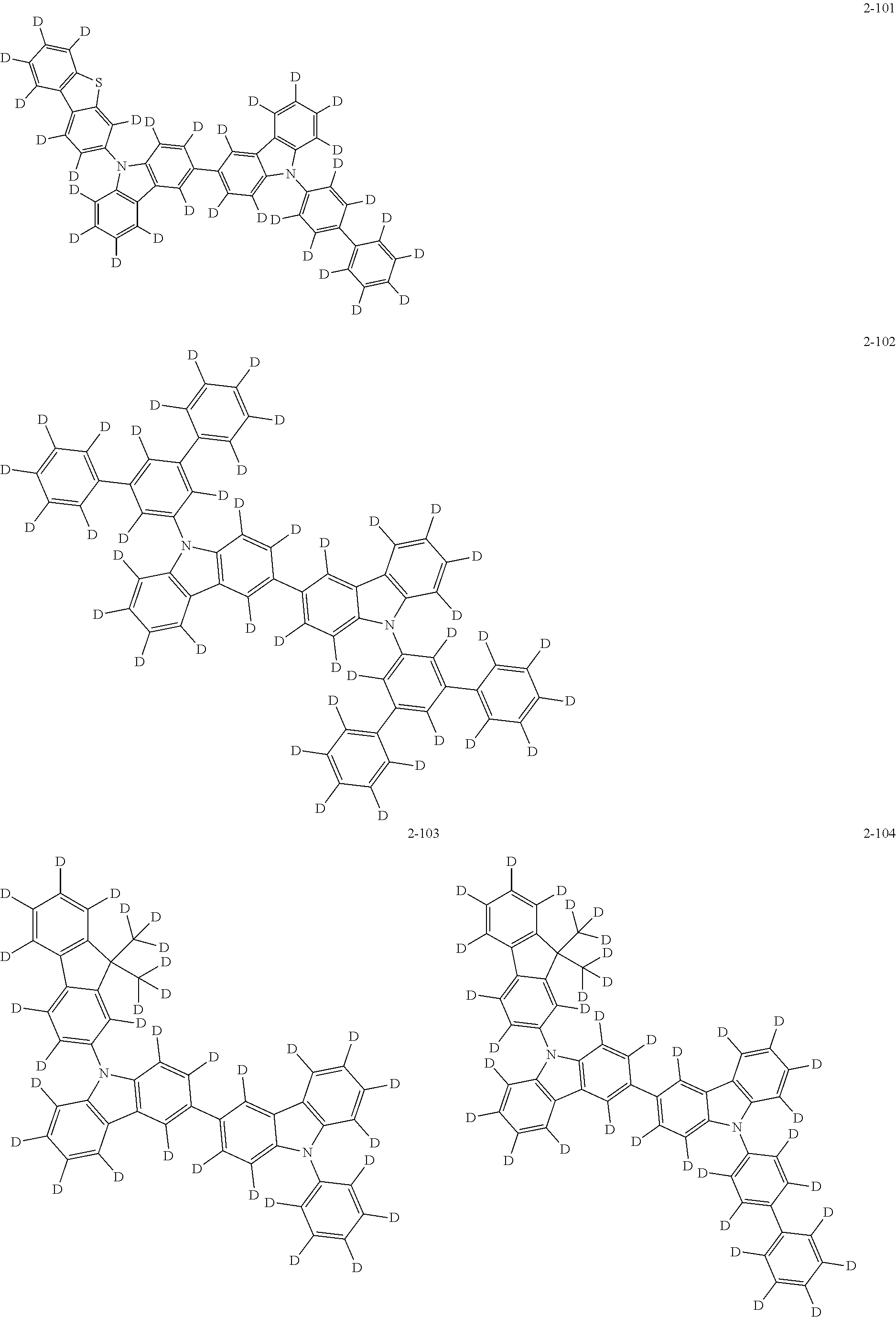

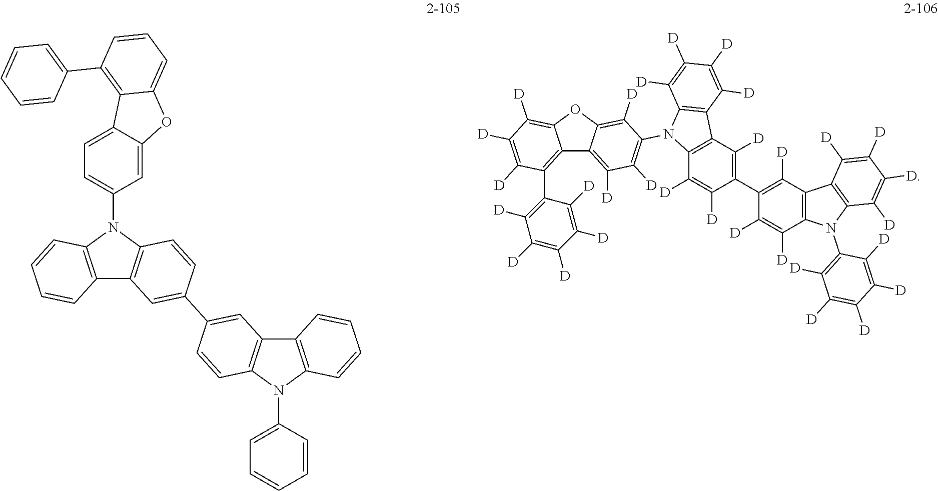

An exemplary embodiment of the present application provides an organic light-emitting device in which Chemical Formula 4 is represented by any one of the following compounds.

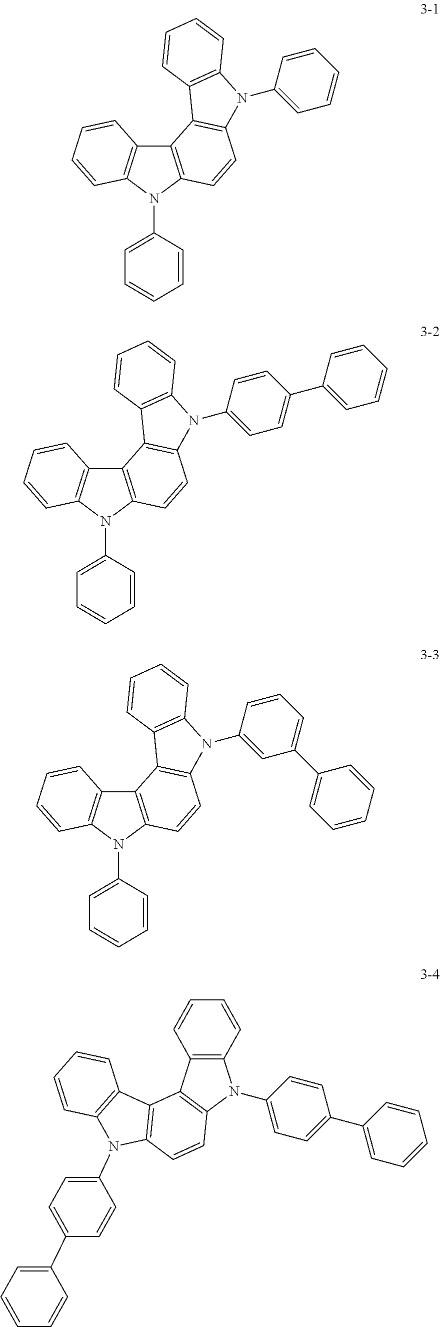

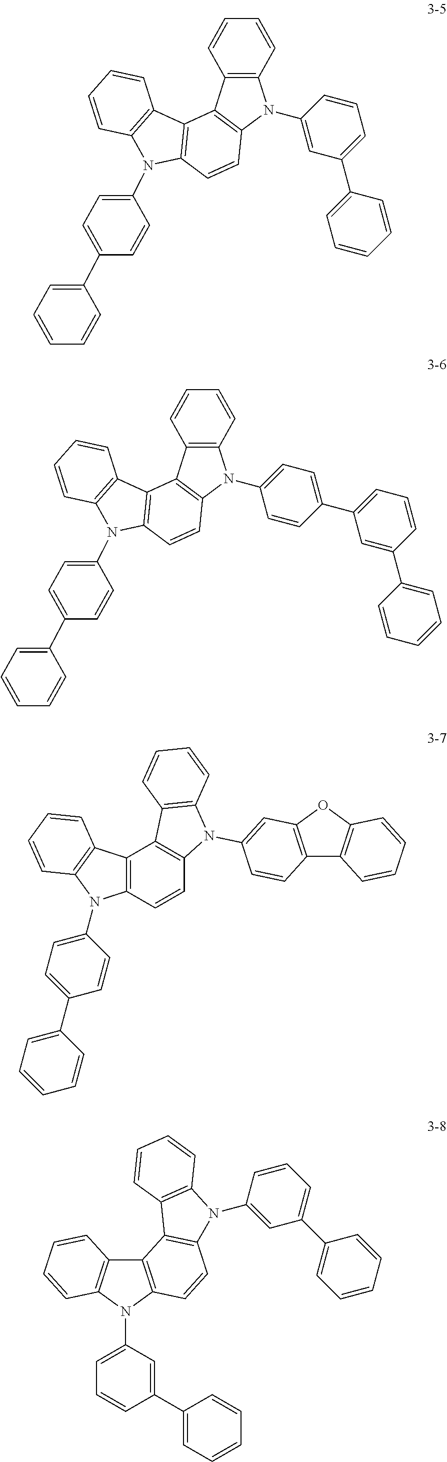 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2cccc(-n3c4ccccc4c4c5c6ccccc6n(-c6cccc(-c7ccccc7)c6)c5ccc43)c2)cc1]

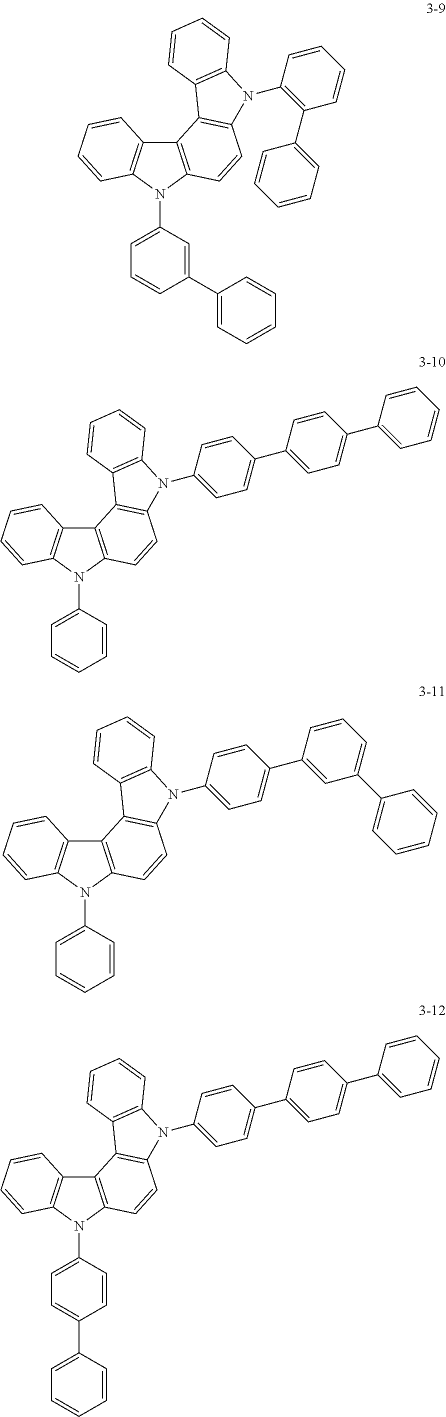 [SMILES: c1ccc(-c2cccc(-n3c4ccccc4c4c5c6ccccc6n(-c6ccccc6-c6ccccc6)c5ccc43)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1 ; c1ccc(-c2ccc(-c3ccc(-n4c5ccccc5c5c6c7ccccc7n(-c7ccc(-c8ccccc8)cc7)c6ccc54)cc3)cc2)cc1]

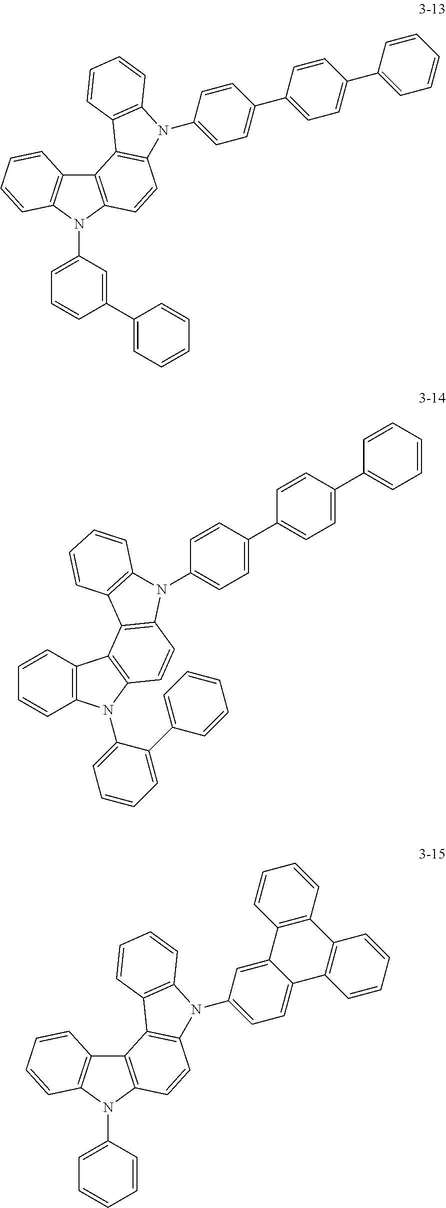 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2ccc(-c3ccc(-n4c5ccccc5c5c6c7ccccc7n(-c7ccccc7-c7ccccc7)c6ccc54)cc3)cc2)cc1 ; c1ccc(-n2c3ccccc3c3c4c5ccccc5n(-c5ccc6c7ccccc7c7ccccc7c6c5)c4ccc32)cc1]

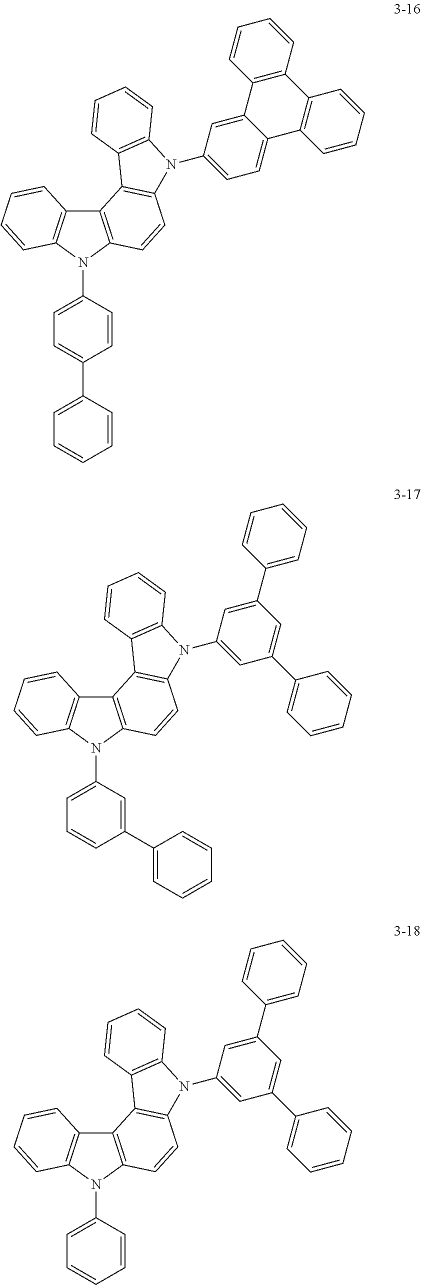 [SMILES: c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6ccccc6)c5ccc43)c2)cc1]

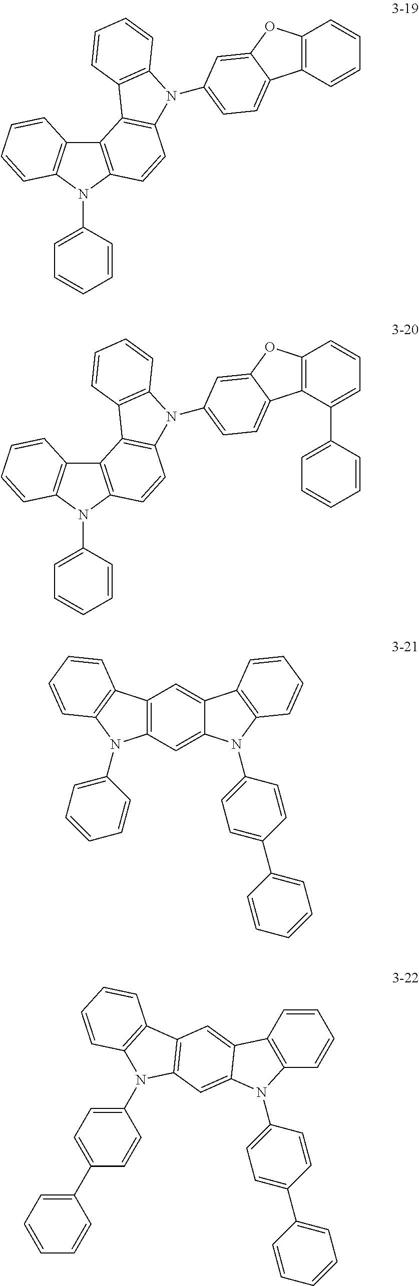

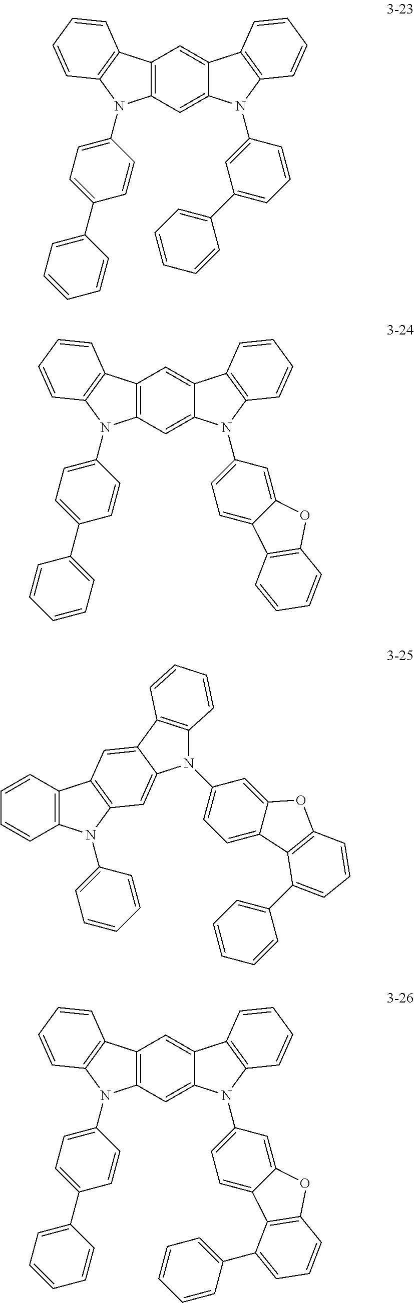

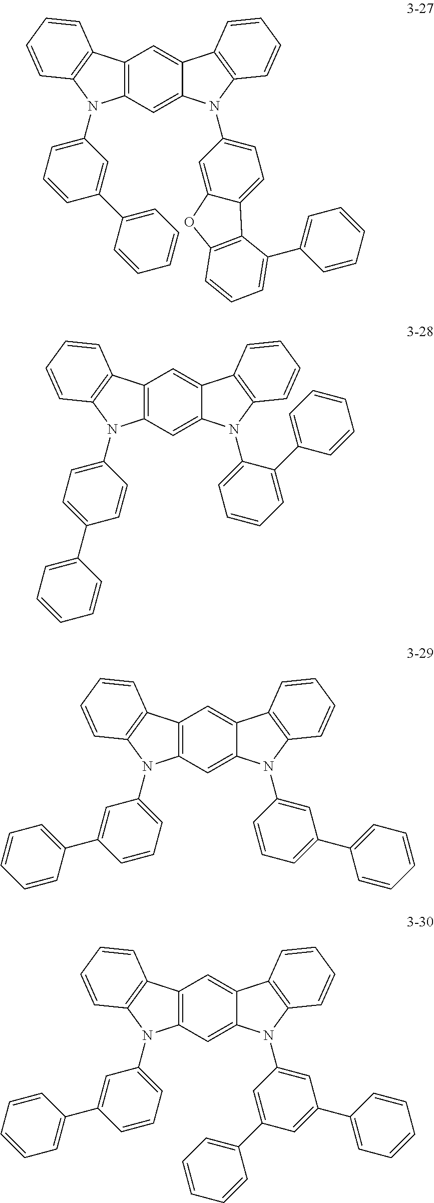

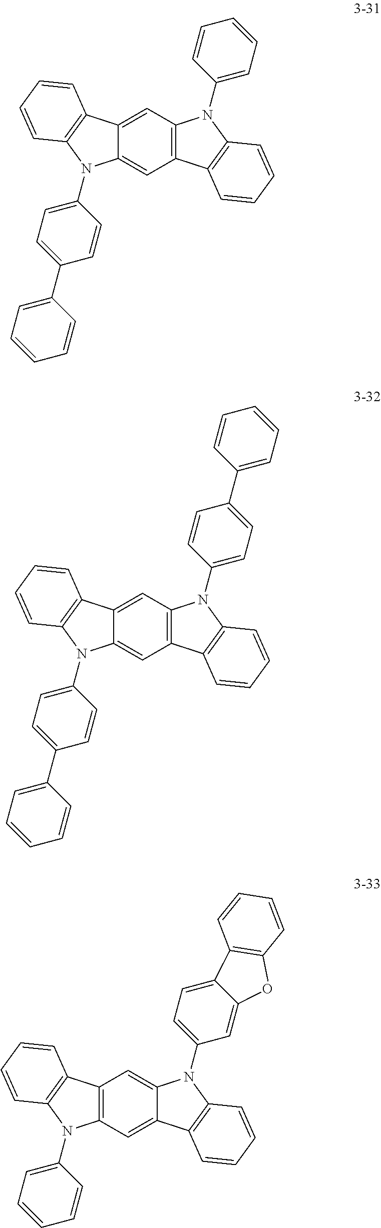 [SMILES: c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1 ; c1ccc(-n2c3ccccc3c3cc4c(cc32)c2ccccc2n4-c2ccc3c(c2)oc2ccccc23)cc1]

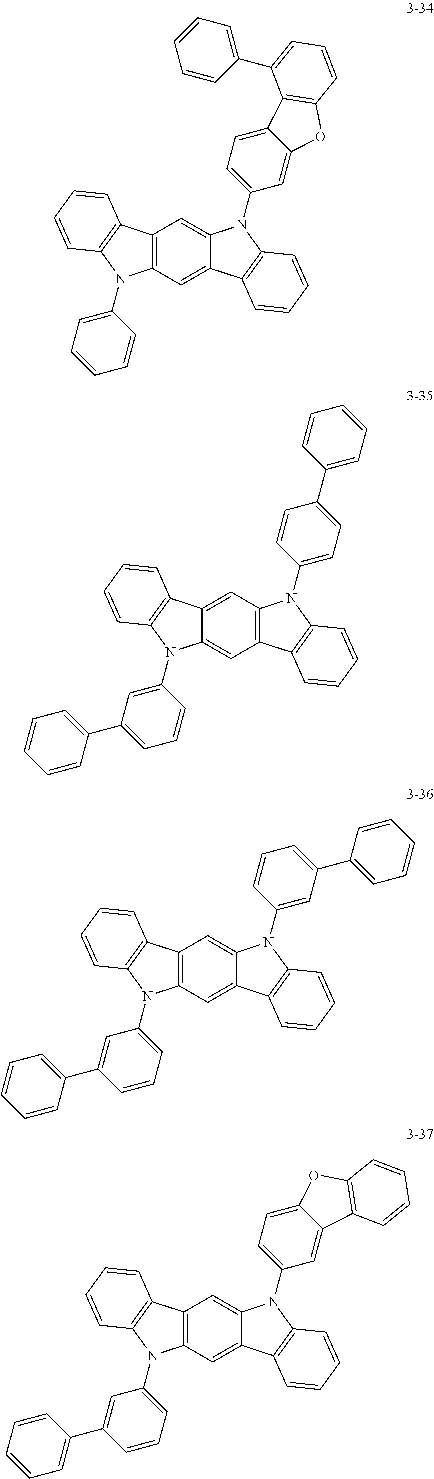 [SMILES: c1ccc(-c2cccc3oc4cc(-n5c6ccccc6c6cc7c(cc65)c5ccccc5n7-c5ccccc5)ccc4c23)cc1 ; c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1 ; c1ccc(-c2cccc(-n3c4ccccc4c4cc5c(cc43)c3ccccc3n5-c3ccc4oc5ccccc5c4c3)c2)cc1]

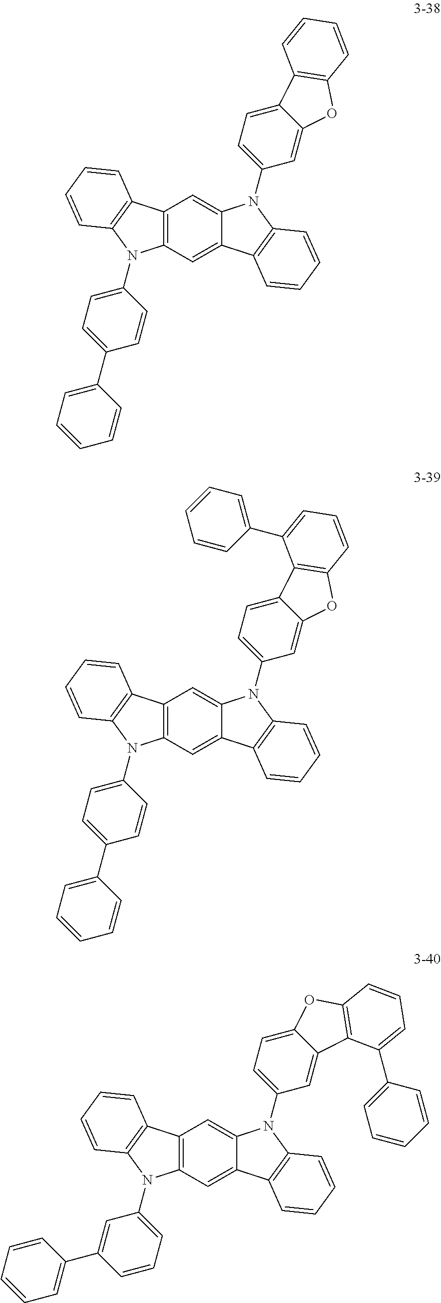 [SMILES: c1ccc(-c2ccc(-n3c4ccccc4c4cc5c(cc43)c3ccccc3n5-c3ccc4c(c3)oc3ccccc34)cc2)cc1 ; c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

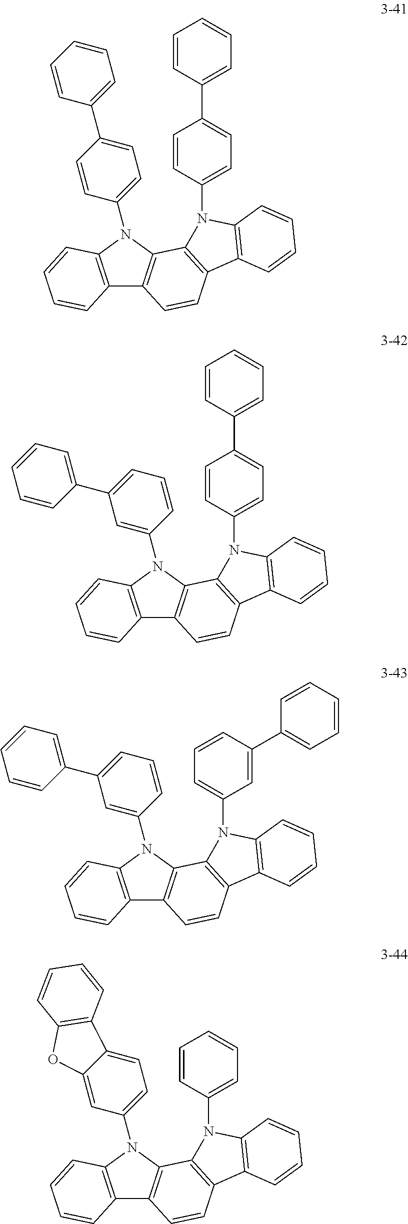 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-n2c3ccccc3c3ccc4c5ccccc5n(-c5ccc6c(c5)oc5ccccc56)c4c32)cc1]

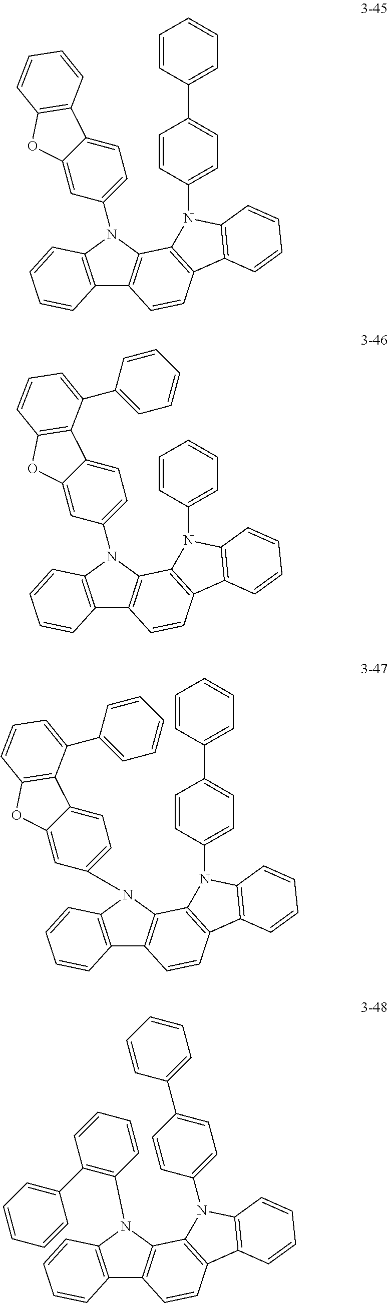

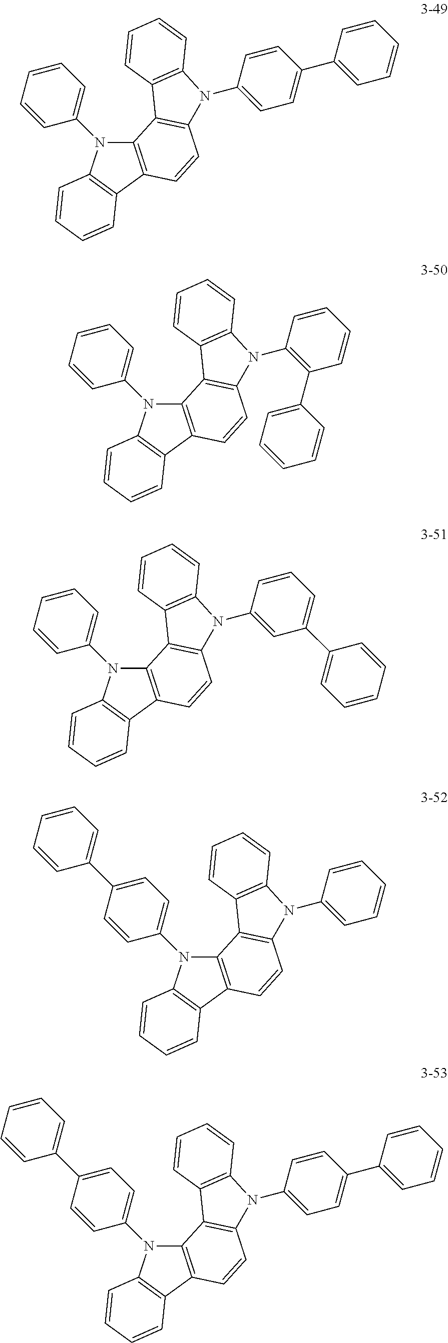

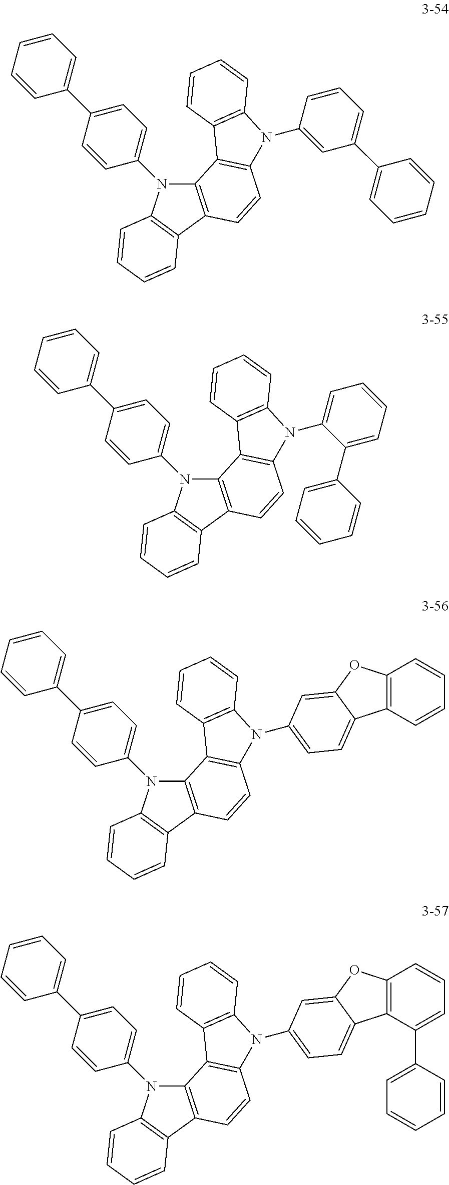 [SMILES: c1ccc(-c2ccc(-n3c4ccccc4c4ccc5c(c6ccccc6n5-c5cccc(-c6ccccc6)c5)c43)cc2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccc6c(c5)oc5ccccc56)c43)cc2)cc1 ; c1ccc(-c2ccc(-n3c4ccccc4c4ccc5c(c6ccccc6n5-c5ccc6c(c5)oc5cccc(-c7ccccc7)c56)c43)cc2)cc1]

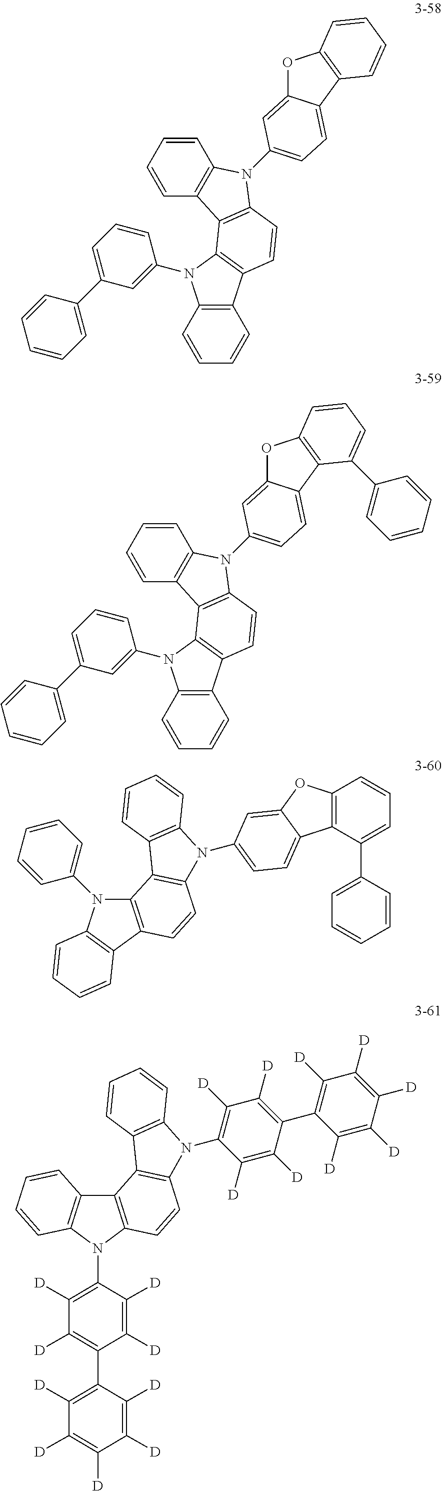

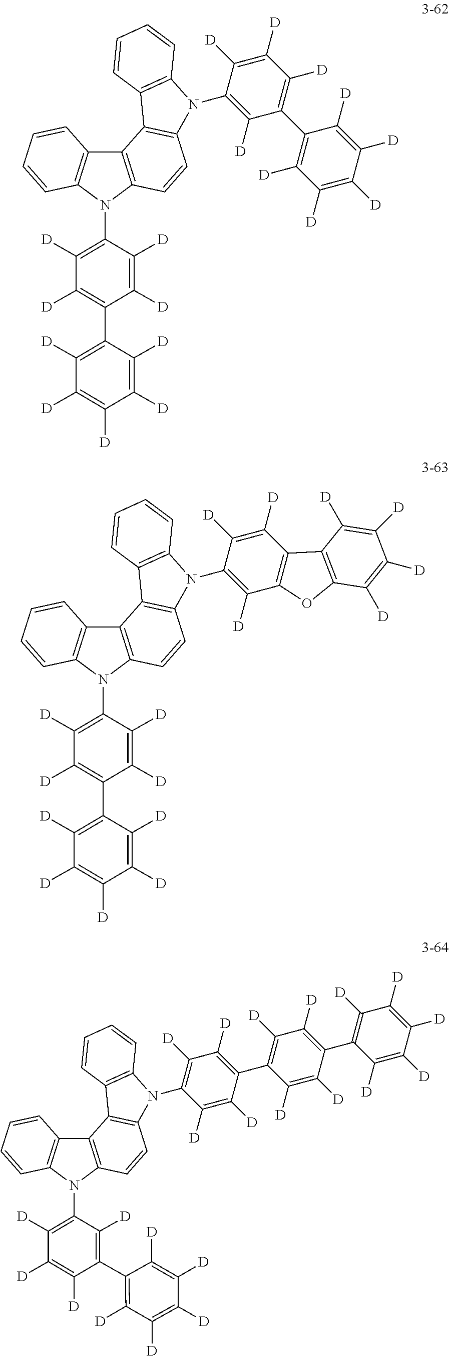

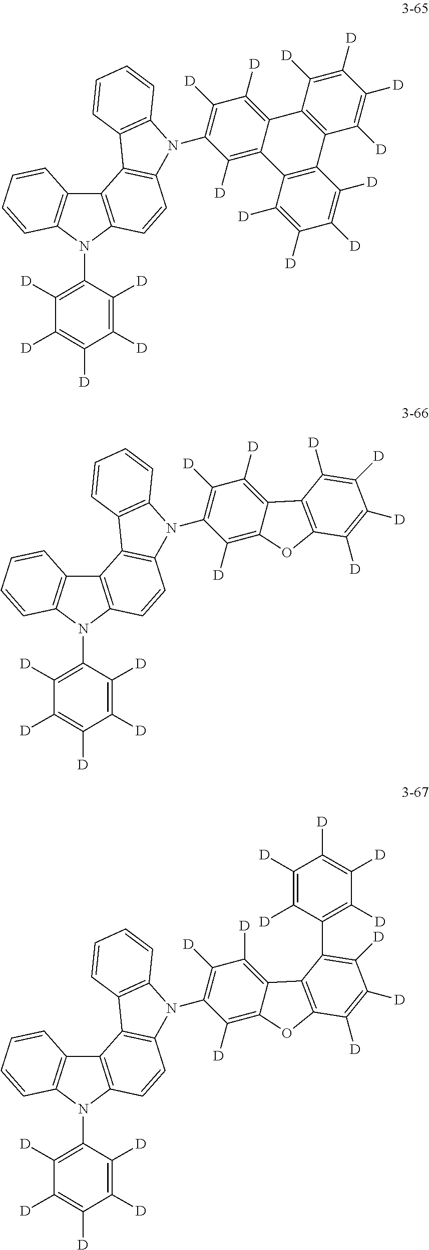 [SMILES: [2H]c1c([2H])c([2H])c(-n2c3ccccc3c3c4c5ccccc5n(-c5c([2H])c([2H])c6c7c([2H])c([2H])c([2H])c([2H])c7c7c([2H])c([2H])c([2H])c([2H])c7c6c5[2H])c4ccc32)c([2H])c1[2H] ; [2H]c1c([2H])c([2H])c(-n2c3ccccc3c3c4c5ccccc5n(-c5c([2H])c([2H])c6c(oc7c([2H])c([2H])c([2H])c([2H])c76)c5[2H])c4ccc32)c([2H])c1[2H] ; [2H]c1c([2H])c([2H])c(-c2c([2H])c([2H])c([2H])c3oc4c([2H])c(-n5c6ccccc6c6c7c8ccccc8n(-c8c([2H])c([2H])c([2H])c([2H])c8[2H])c7ccc65)c([2H])c([2H])c4c23)c([2H])c1[2H]]

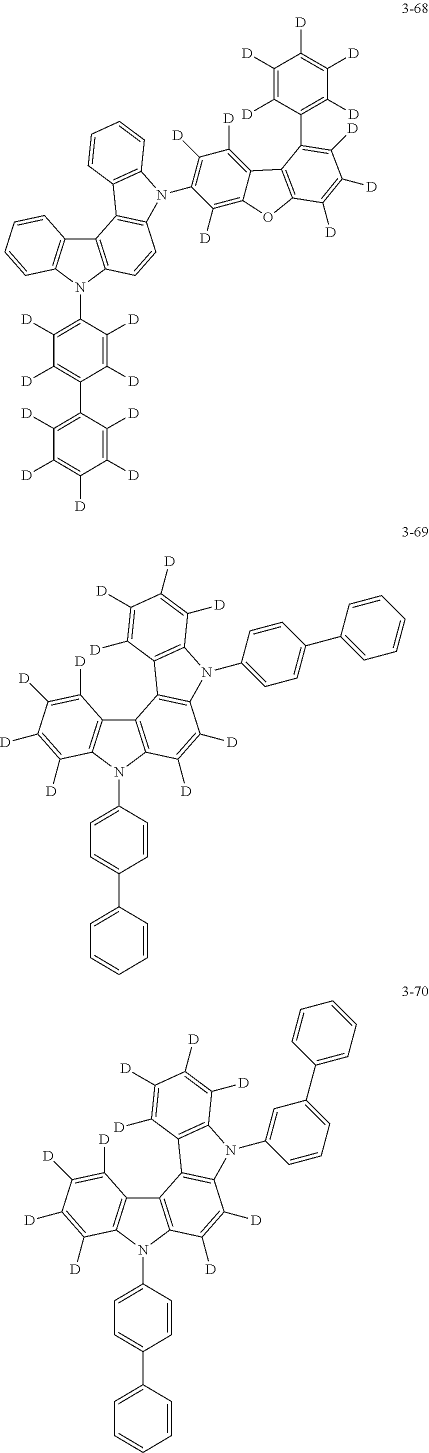 [SMILES: [2H]c1c([2H])c([2H])c(-c2c([2H])c([2H])c(-n3c4ccccc4c4c5c6ccccc6n(-c6c([2H])c([2H])c7c(oc8c([2H])c([2H])c([2H])c(-c9c([2H])c([2H])c([2H])c([2H])c9[2H])c87)c6[2H])c5ccc43)c([2H])c2[2H])c([2H])c1[2H] ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1 ; [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1ccc(-c2ccccc2)cc1]

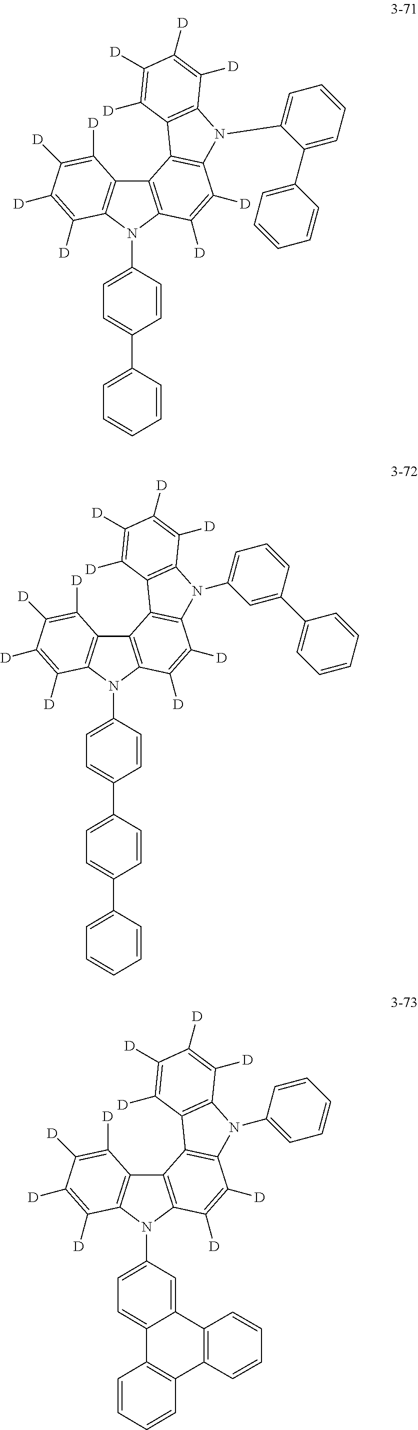 [SMILES: [2H]c1c([2H])c([2H])c2c(c1[2H])c1c3c4c([2H])c([2H])c([2H])c([2H])c4n(-c4ccccc4-c4ccccc4)c3c([2H])c([2H])c1n2-c1ccc(-c2ccccc2)cc1 ; [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1ccc(-c2ccc(-c3ccccc3)cc2)cc1 ; [2H]c1c([2H])c([2H])c2c(c1[2H])c1c3c4c([2H])c([2H])c([2H])c([2H])c4n(-c4ccc5c6ccccc6c6ccccc6c5c4)c3c([2H])c([2H])c1n2-c1ccccc1]

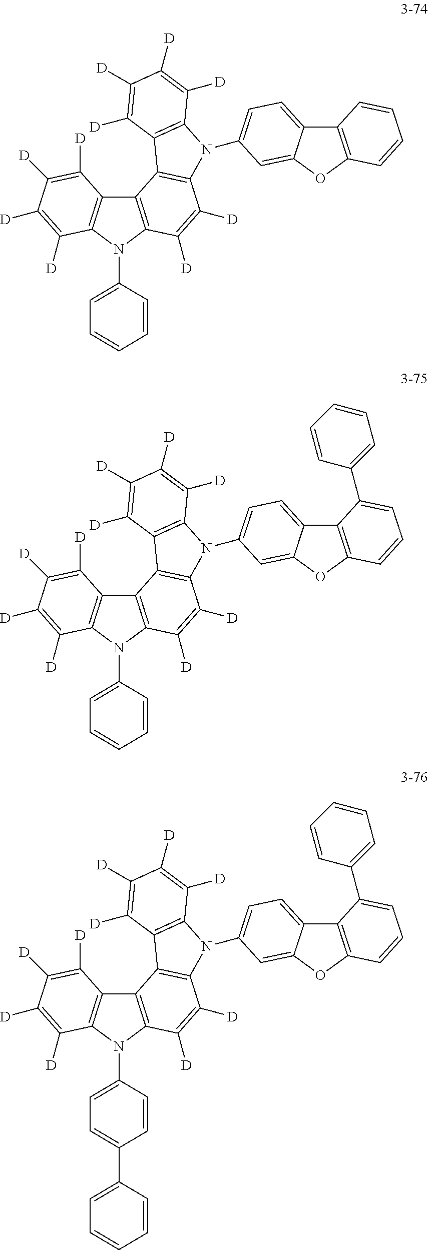 [SMILES: [2H]c1c([2H])c([2H])c2c(c1[2H])c1c3c4c([2H])c([2H])c([2H])c([2H])c4n(-c4ccc5c(c4)oc4ccccc45)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4cccc(-c6ccccc6)c45)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4cccc(-c6ccccc6)c45)c3c([2H])c([2H])c1n2-c1ccc(-c2ccccc2)cc1]

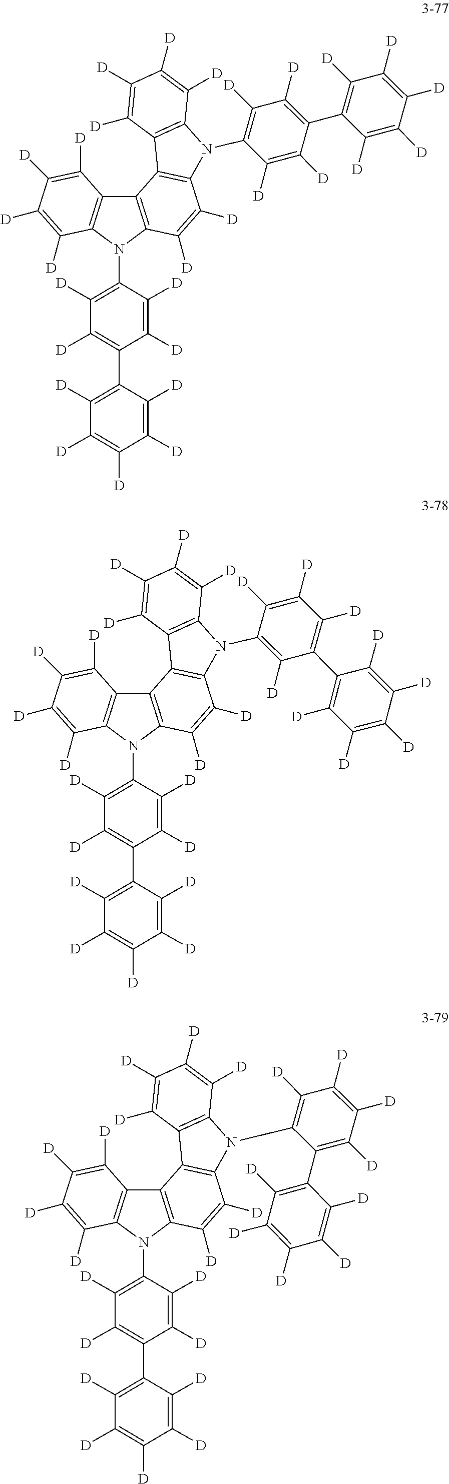

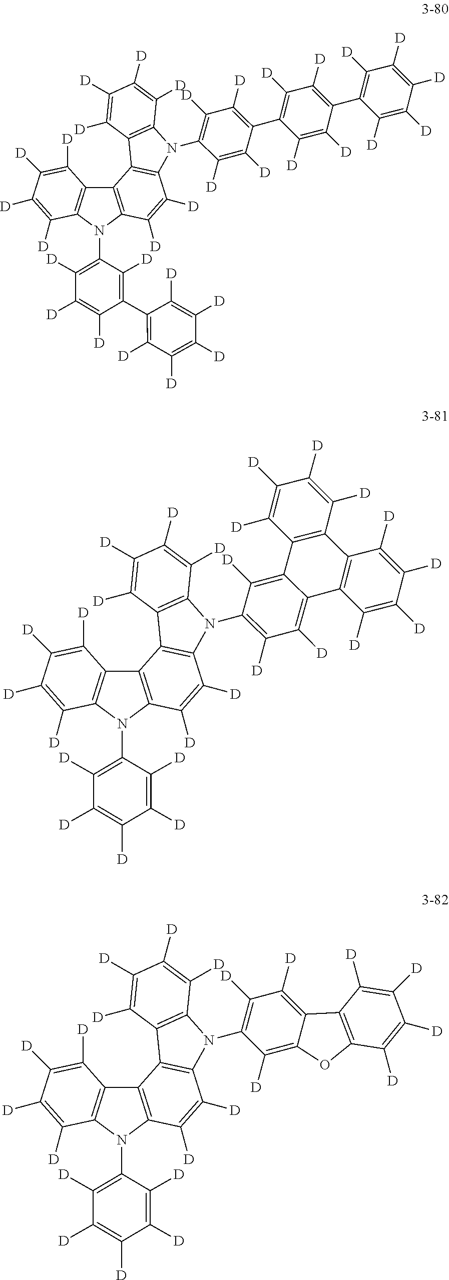

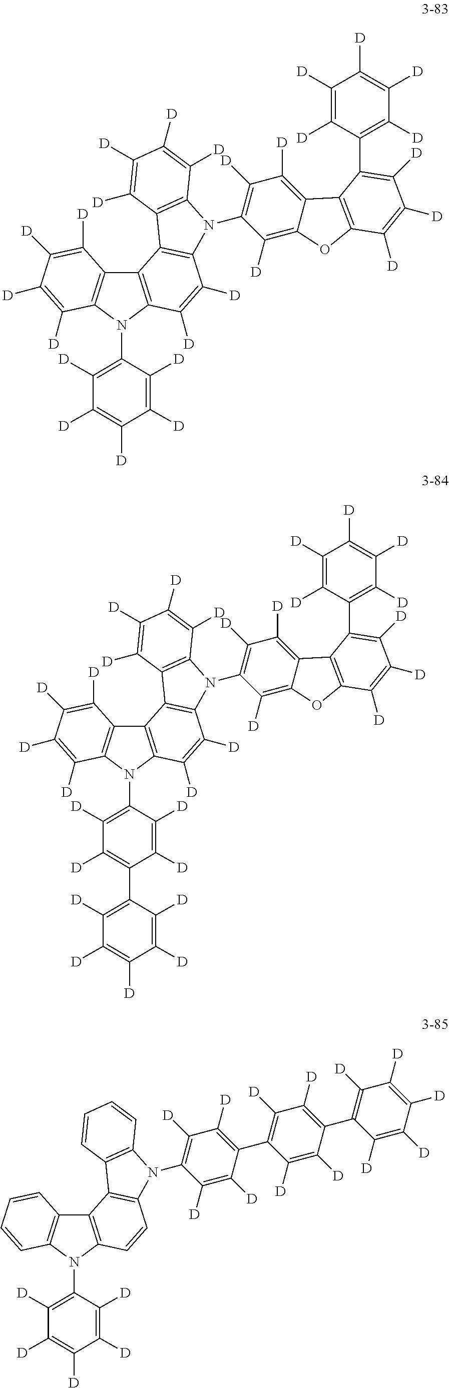

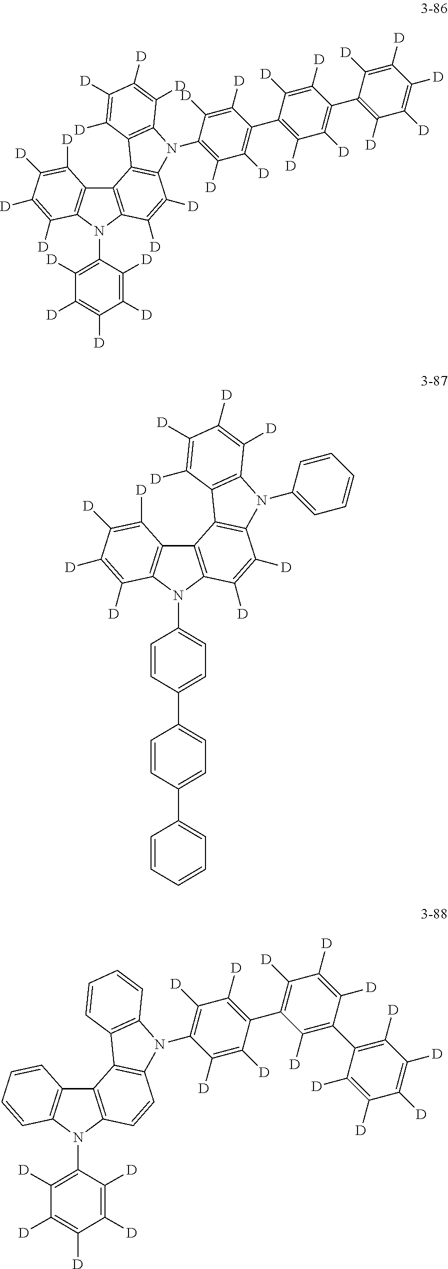

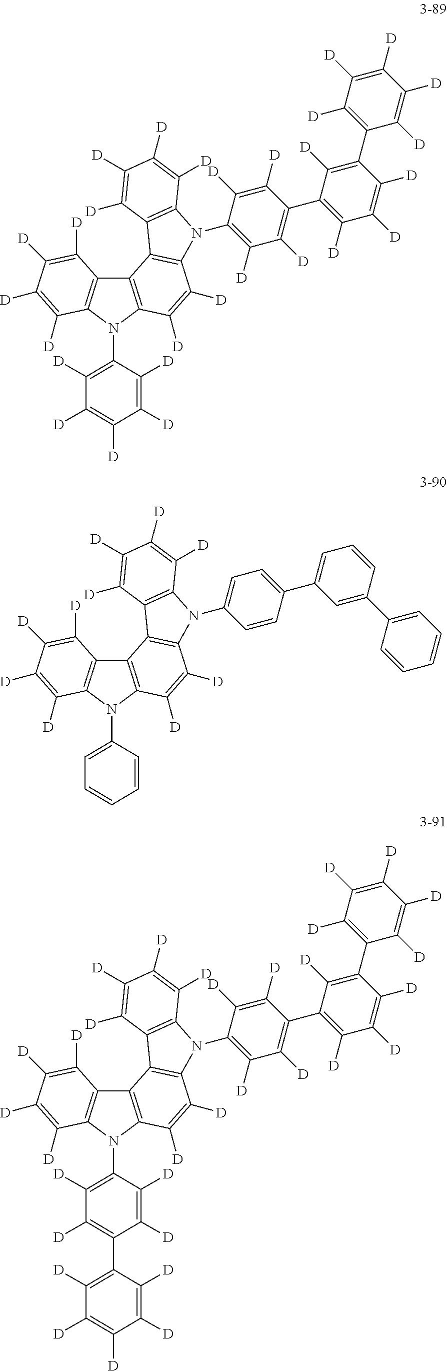

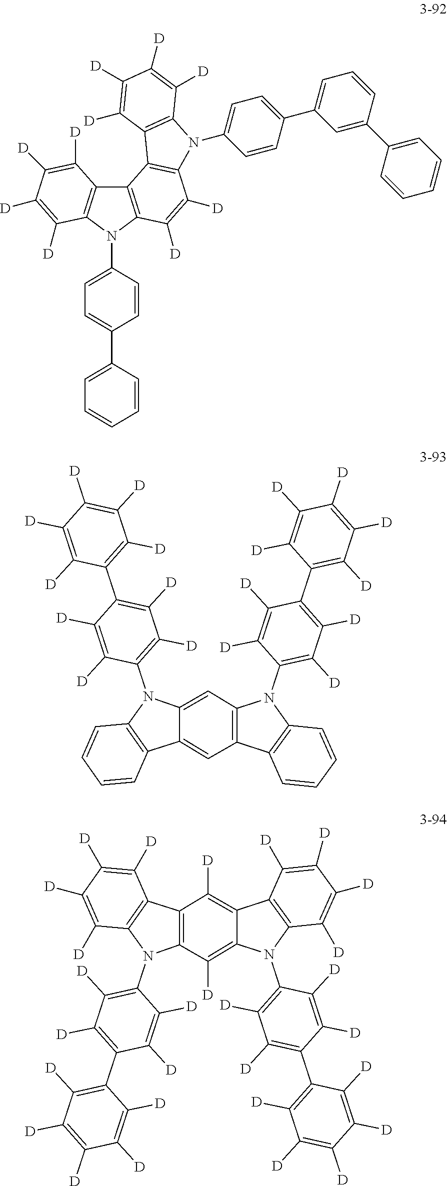

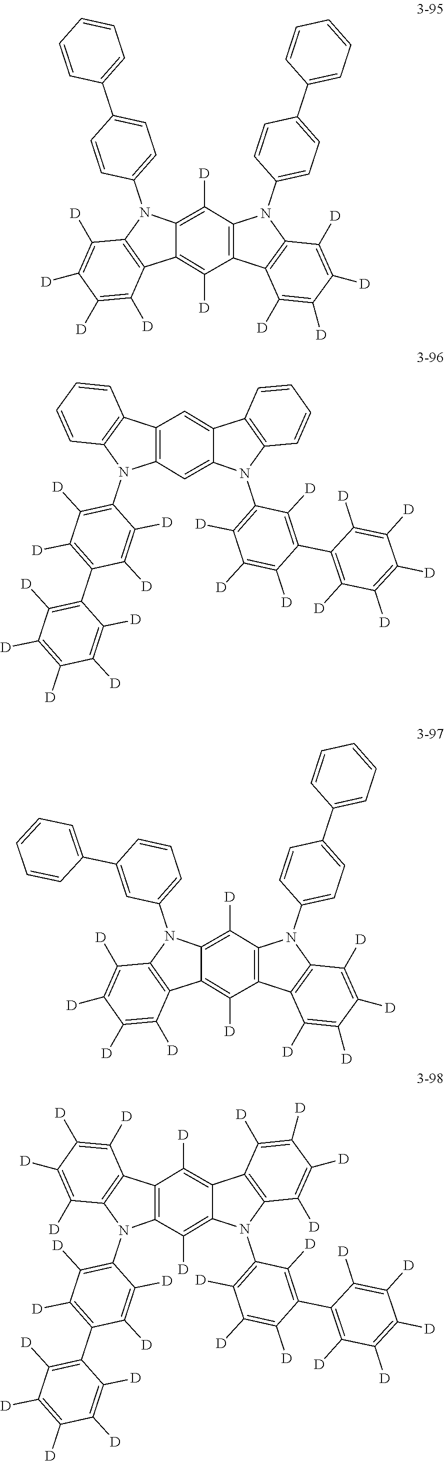

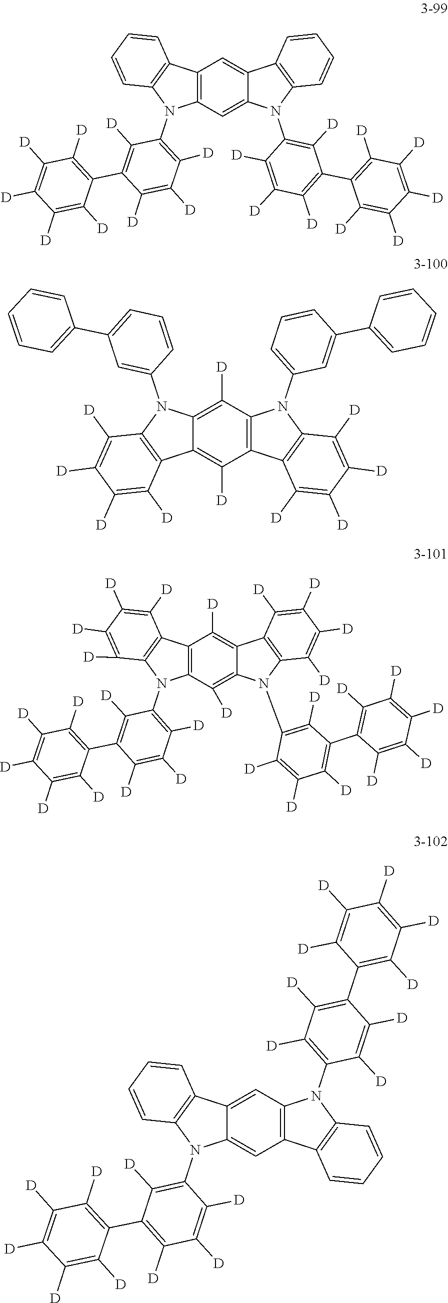

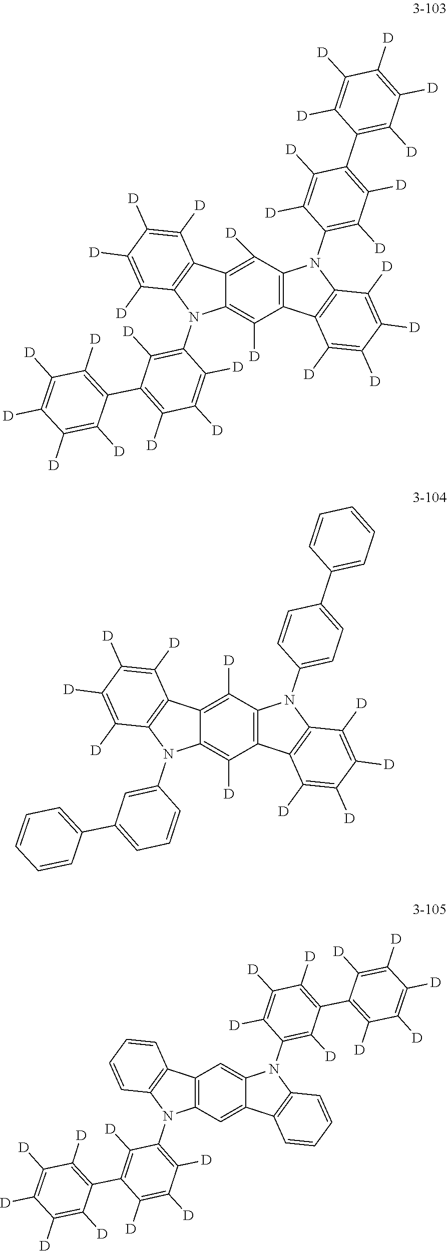

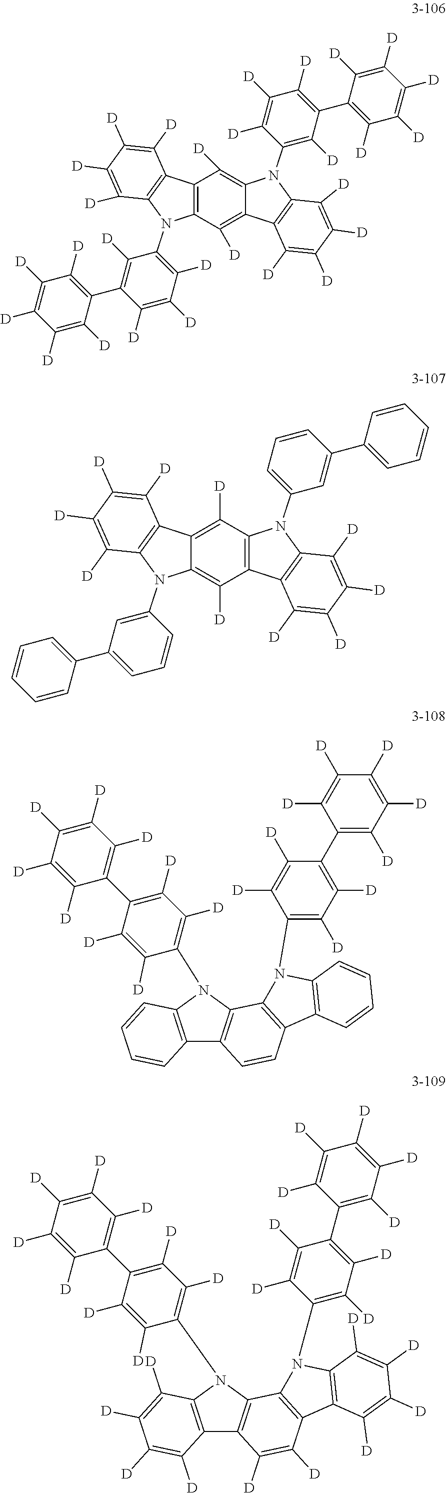

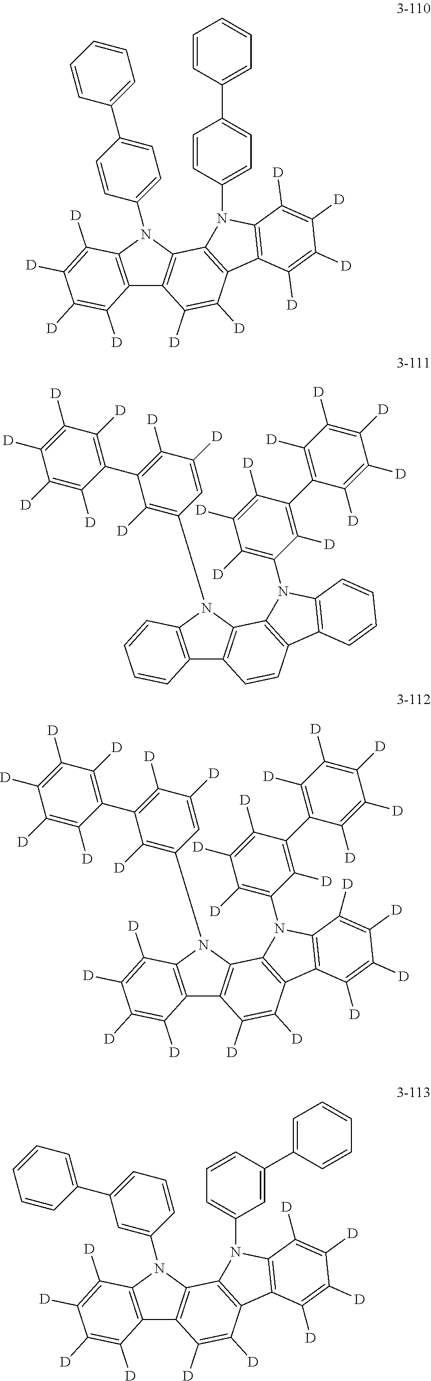

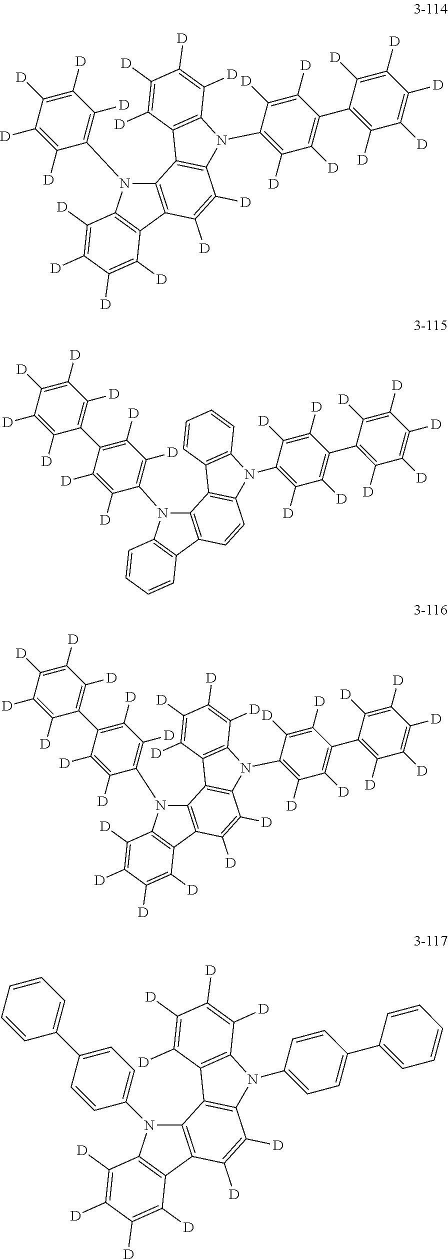

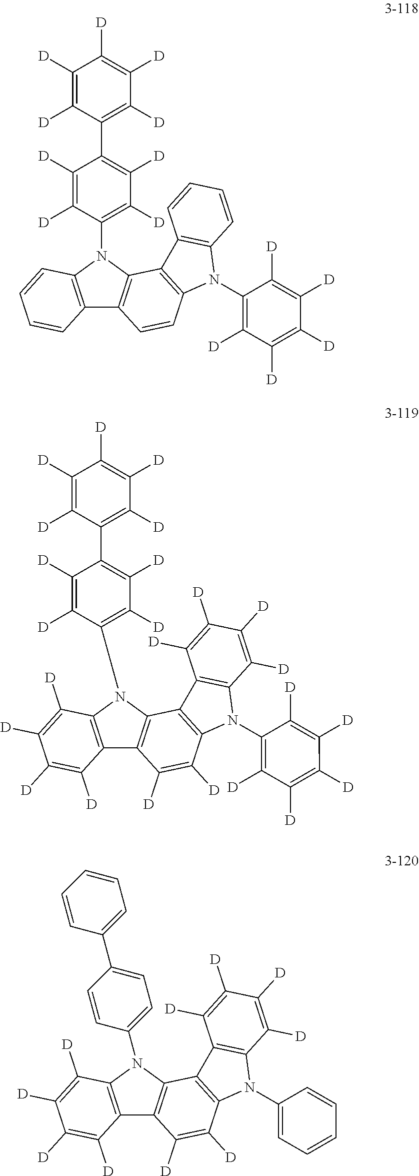 [SMILES: [2H]c1c([2H])c([2H])c(-c2c([2H])c([2H])c(-n3c4ccccc4c4ccc5c(c6ccccc6n5-c5c([2H])c([2H])c([2H])c([2H])c5[2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c([2H])c5c(c6c([2H])c([2H])c([2H])c([2H])c6n5-c5c([2H])c([2H])c([2H])c([2H])c5[2H])c43)c([2H])c2[2H])c([2H])c1[2H] ; [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccccc1]

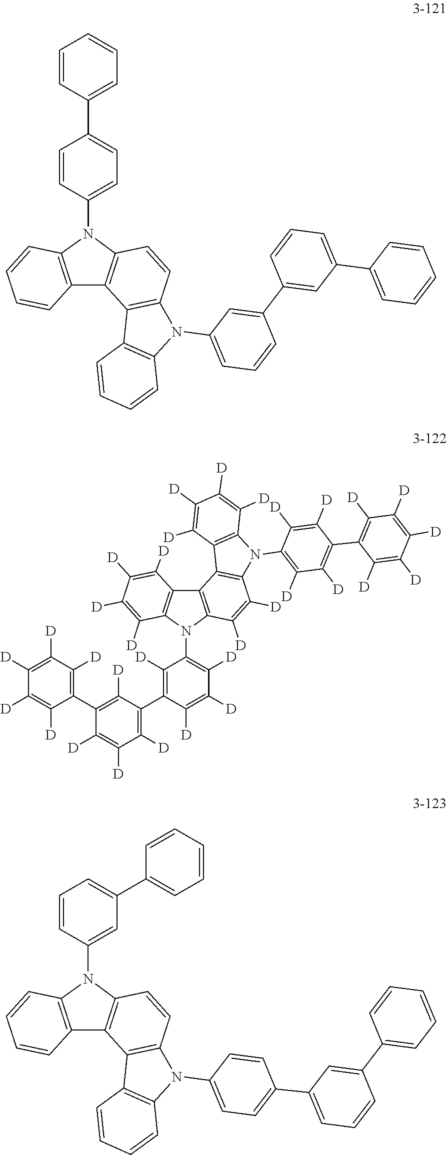 [SMILES: c1ccc(-c2ccc(-n3c4ccccc4c4c5c6ccccc6n(-c6cccc(-c7cccc(-c8ccccc8)c7)c6)c5ccc43)cc2)cc1 ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c(-c8c([2H])c([2H])c([2H])c([2H])c8[2H])c7[2H])c6[2H])c5c([2H])c([2H])c43)c([2H])c2[2H])c([2H])c1[2H] ; c1ccc(-c2cccc(-c3ccc(-n4c5ccccc5c5c6c7ccccc7n(-c7cccc(-c8ccccc8)c7)c6ccc54)cc3)c2)cc1]

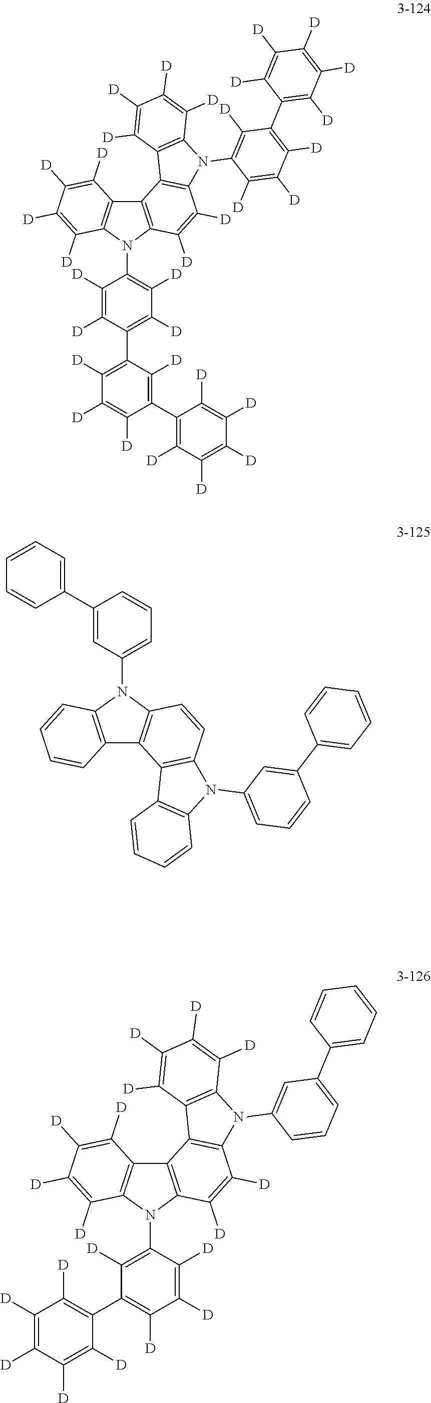

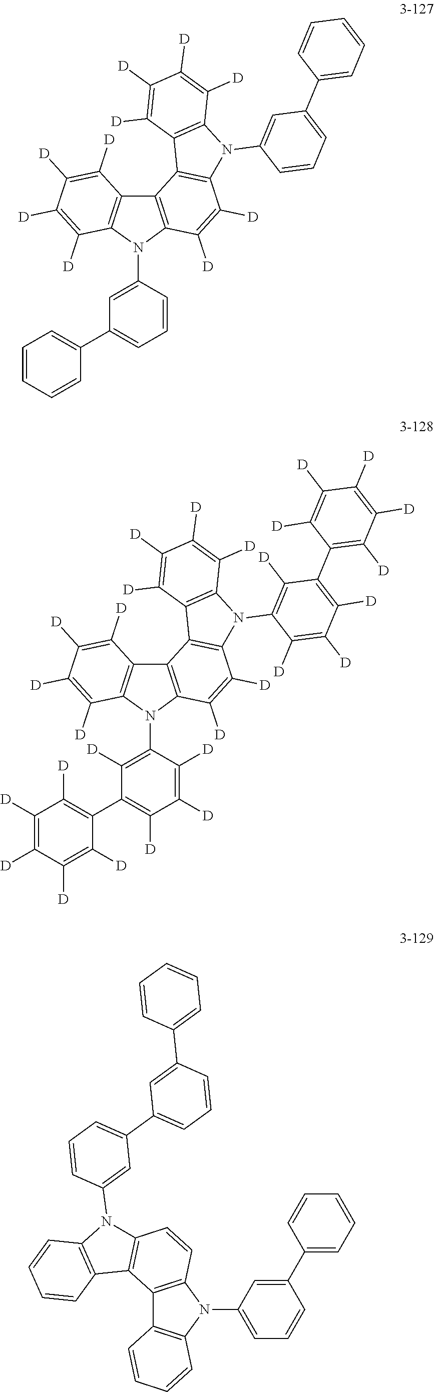 [SMILES: [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1cccc(-c2ccccc2)c1 ; [2H]c1c([2H])c([2H])c(-c2c([2H])c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c2[2H])c([2H])c1[2H] ; c1ccc(-c2cccc(-c3cccc(-n4c5ccccc5c5c6c7ccccc7n(-c7cccc(-c8ccccc8)c7)c6ccc54)c3)c2)cc1]

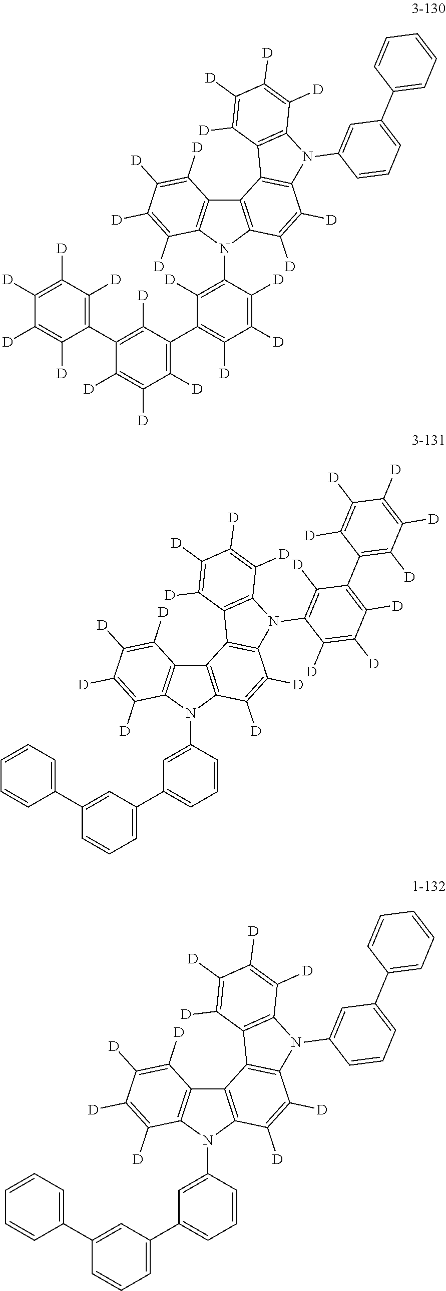

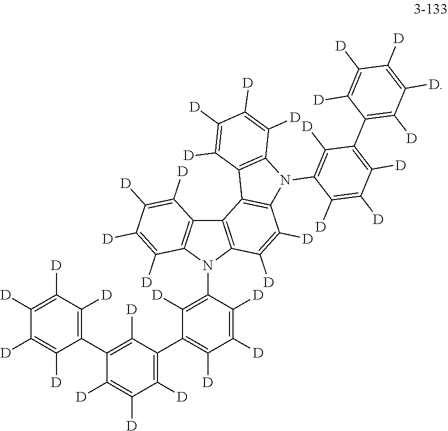

The descriptions regarding the organic light-emitting device comprising the heterocyclic compound represented by Chemical Formula 1 described above may be applied to the organic light-emitting device further comprising the heterocyclic compound represented by Chemical Formula 3 or 4.

In another exemplary embodiment, the heterocyclic compound represented by Chemical Formula 3 or 4 may be used as a light-emitting material of a light-emitting layer of an organic light-emitting device.

In another exemplary embodiment, the heterocyclic compound represented by Chemical Formula 3 or 4 may be used as a light-emitting material of a light-emitting layer of an organic light-emitting device, and may be used as a p-type host material.

In the organic light-emitting device of the present invention, the organic material layer may comprise the heterocyclic compound represented by Chemical Formula 1 and the heterocyclic compound represented by Chemical Formula 3 or Chemical Formula 4. The organic material layer may be formed using a thermal vacuum deposition method by pre-mixing the heterocyclic compound represented by Chemical Formula 1 and the heterocyclic compound represented by Chemical Formula 3 or 4.

In another organic light-emitting device, the organic material layer may comprise a light-emitting layer, the light-emitting layer may comprise a host material, and the host material may comprise the heterocyclic compound represented by Chemical Formula 1 and the heterocyclic compound represented by Chemical Formula 3 or 4.

In another organic light-emitting device, the organic material layer may comprise a light-emitting layer, and may comprise the heterocyclic compound represented by Chemical Formula 1 as an n-type host material of the light-emitting layer, and the heterocyclic compound represented by Chemical Formula 3 or Chemical Formula 4 as a p-type host material.

An exemplary embodiment of the present application provides a method for manufacturing an organic light-emitting device, the method comprising: preparing a substrate; forming a first electrode on the substrate; forming one or more organic material layers on the first electrode; and forming a second electrode on the organic material layers, wherein the forming of the organic material layers comprises forming one or more organic material layers using a composition for an organic material layer according to an exemplary embodiment of the present application.

An exemplary embodiment of the present application provides a method for manufacturing an organic light-emitting device, wherein the forming of the organic material layer is forming using a thermal vacuum deposition method after supplying the heterocyclic compound represented by Chemical Formula 1 and the heterocyclic compound represented by Chemical Formula 3 or Chemical Formula 4 to respective individual sources.

An exemplary embodiment of the present application provides a method for manufacturing an organic light-emitting device, wherein the forming of the organic material layer is forming using a thermal vacuum deposition method by pre-mixing the heterocyclic compound represented by Chemical Formula 1 and the heterocyclic compound represented by Chemical Formula 3 or 4.

The organic light-emitting device according to an exemplary embodiment of the present application may be manufactured by a conventional manufacturing method and material for an organic light-emitting device, except that an organic material layer is formed using the aforementioned heterocyclic compound.

The organic light-emitting device of the present invention may further comprise one or more layers selected from the group consisting of a light-emitting layer, a hole injection layer, a hole transport layer, an electron blocking layer, an electron injection layer, an electron transport layer, a hole auxiliary layer, and a hole blocking layer.

An exemplary embodiment of the present application provides a composition for an organic material layer of an organic light-emitting device, the composition comprising the heterocyclic compound represented by Chemical Formula 1 and the heterocyclic compound represented by Chemical Formula 3 or 4.

A weight ratio of the compound represented by Chemical Formula 1 and the compound represented by Chemical Formula 3 may be 1:1 to 1:10, or a weight ratio of the compound represented by Chemical Formula 1 and the compound represented by Chemical Formula 4 may be 1:1 to 1:10.

A weight ratio of the heterocyclic compound represented by Chemical Formula 1: the heterocyclic compound represented by Chemical Formula 3 in the composition may be 1:1 to 1:8, may be 1:1 to 1:5, may be 1:1 to 1:3, and may be 1:1 to 1:2, but is not limited thereto.

A weight ratio of the heterocyclic compound represented by Chemical Formula 1: the heterocyclic compound represented by Chemical Formula 4 in the composition may be 1:1 to 1:8, may be 1:1 to 1:5, may be 1:1 to 1:3, and may be 1:1 to 1:2, but is not limited thereto.

The composition may be used when forming an organic material layer of an organic light-emitting device, and in particular, may be more preferably used as a host material of a light-emitting layer.

The composition is in a form in which two or more compounds are simply mixed, and materials in a powder state may be mixed before forming the organic material layer of the organic light-emitting device, or compounds in a liquid state at an appropriate temperature or higher may be mixed. The composition is in a solid state below a melting point of each material, and may be maintained in a liquid phase by adjusting a temperature.

The composition may additionally comprise materials known in the art, such as a solvent, an additive, and the like.

Hereinafter, the present specification will be described in more detail through examples, but these are only for illustrating the present application and are not intended to limit the scope of the present application.

### SYNTHESIS EXAMPLE

### [Preparation Example 1] Preparation of Compound 1-1 [C]

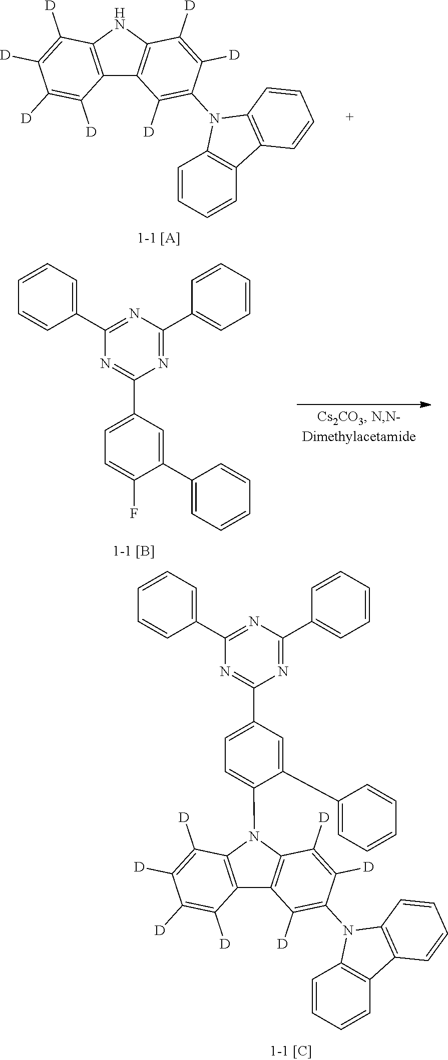 [SMILES: [2H]c1c([2H])c([2H])c2c([nH]c3c([2H])c([2H])c(-n4c5ccccc5c5ccccc54)c([2H])c32)c1[2H] ; Fc1ccc(-c2nc(-c3ccccc3)nc(-c3ccccc3)n2)cc1-c1ccccc1 ; [2H]c1c([2H])c([2H])c2c(c1[2H])c1c([2H])c(-n3c4ccccc4c4ccccc43)c([2H])c([2H])c1n2-c1ccc(-c2nc(-c3ccccc3)nc(-c3ccccc3)n2)cc1-c1ccccc1]

15 g (44.32 mmol) of 6-carbazol-9-yl-1,2,3,4,7,8-hexadeuterio-9H-carbazole [1-1-A], 18.78 g (46.54 mmol) of 2-(4-fluoro-3-phenyl-phenyl)-4,6-diphenyl-1,3,5-triazine [1-1-B], 28.88 g (88.64 mmol) of Cs₂CO₃, and 200 mL of dimethylacetamide were added to a one-neck round bottom flask and reacted for 4 hours while stirring at a reflux temperature. After completion of the reaction, the mixture was worked up using methylene chloride and water, and then an organic layer was concentrated.

The concentrated product was dissolved in methylene chloride, passed through a silica filter, and then a filtrate was concentrated. After a slurry treatment using acetone and methanol and filtering, 24 g (33.25 mmol) of the target compound 6-carbazol-9-yl-1,2,3,4,7,8-hexadeuterio-9-[4-(4,6-diphenyl-1,3,5-triazin-2-yl)-2-phenyl-phenyl]carbazole (Yield: 75%) 1-1[C] was obtained.

In Preparation Example 1, a target compound C was prepared in the same manner except that [A] and [B] were respectively replaced with Intermediates A and B of Table 1 below.

TABLE 1

| com- | Reactant | Reactant |
|---|---|---|
| pound | A | B |
| 1-1 | 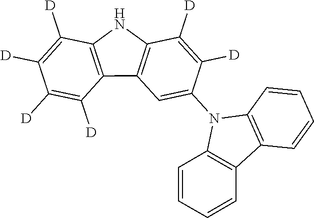 | 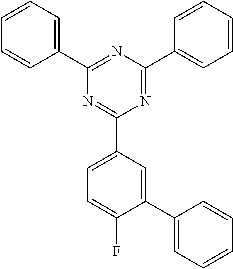 |
| 1-2 | 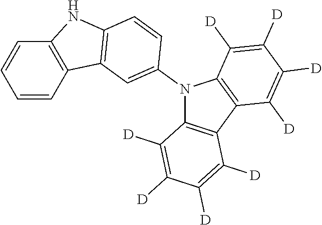 | 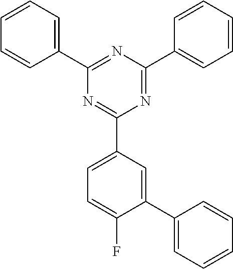 |
| 1-3 | 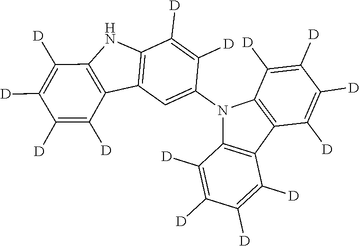 | 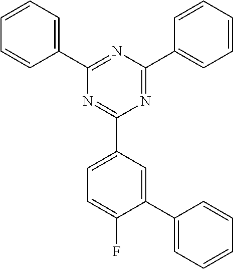 |
| 1-4 | 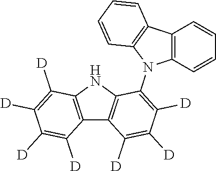 | 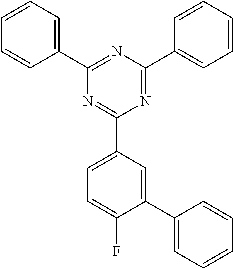 |
| 1-5 | 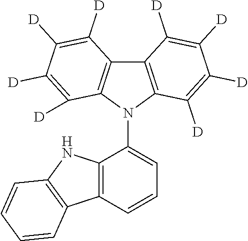 | 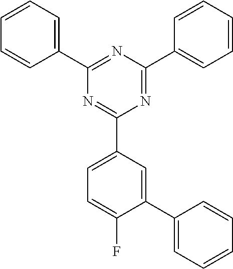 |
| 1-6 | 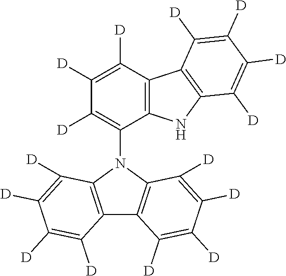 | 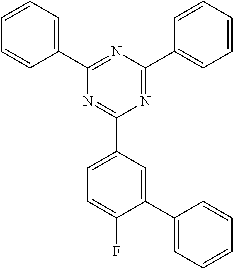 |
| 1-7 | 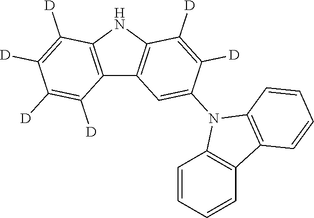 | 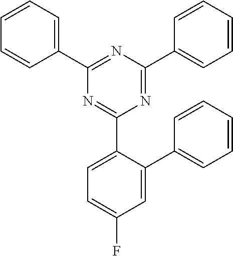 |
| 1-8 | 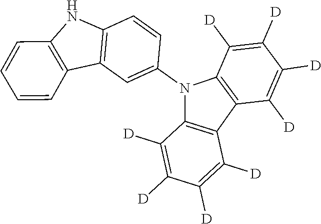 | 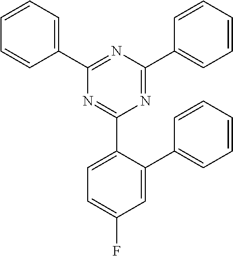 |
| 1-9 | 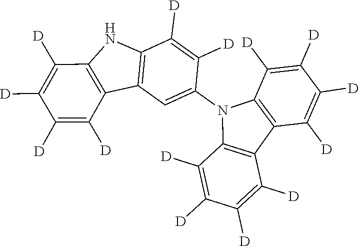 | 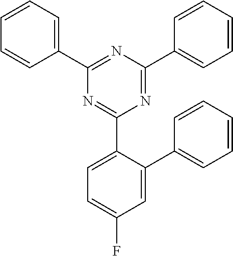 |
| 1-10 | 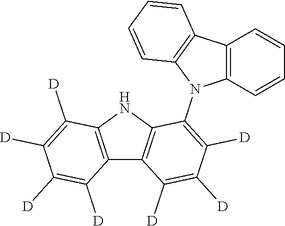 | 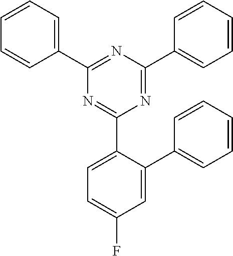 |
| 1-11 | 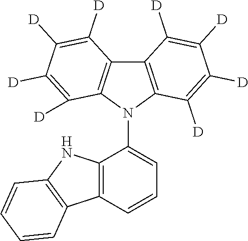 | 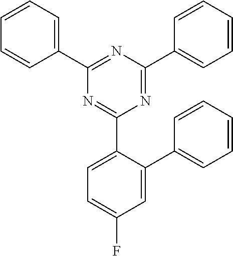 |
| 1-12 | 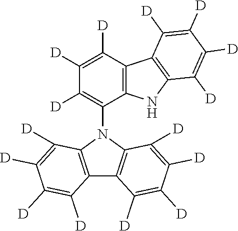 | 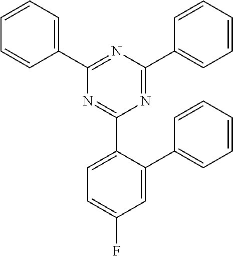 |
| 1-25 | 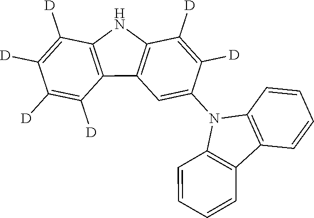 | 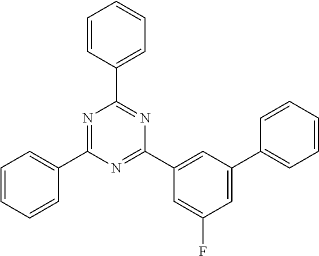 |
| 1-26 | 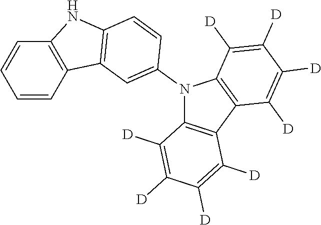 | 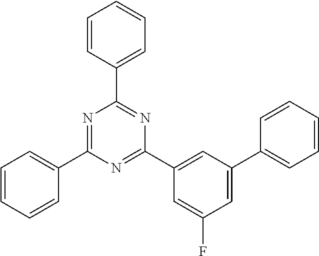 |
| 1-27 | 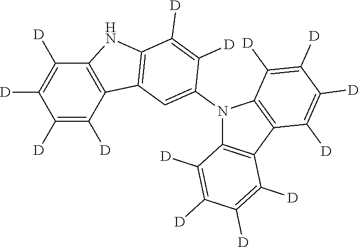 | 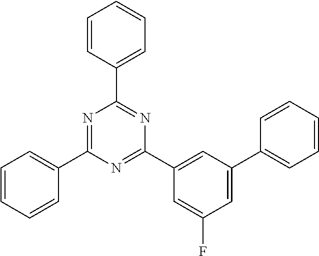 |
| 1-28 | 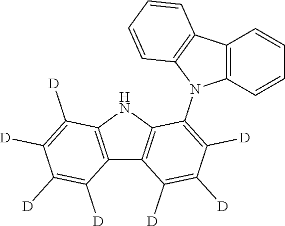 | 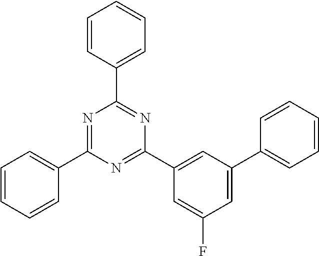 |
| 1-29 | 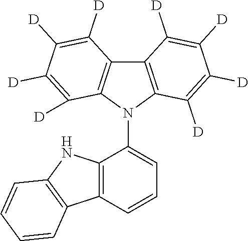 | 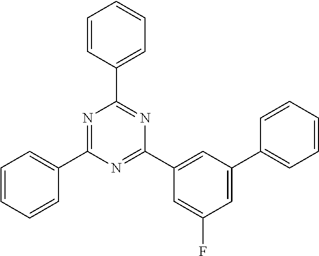 |
| 1-30 | 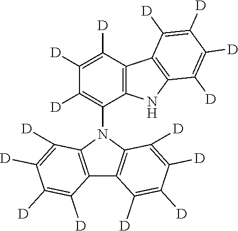 | 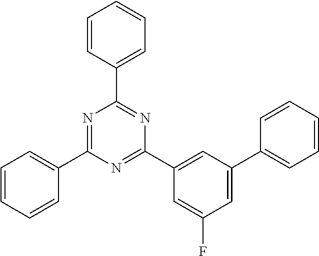 |
| 1-49 | 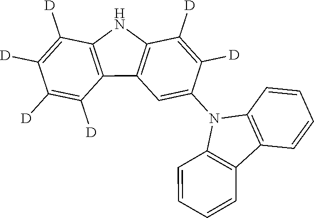 | 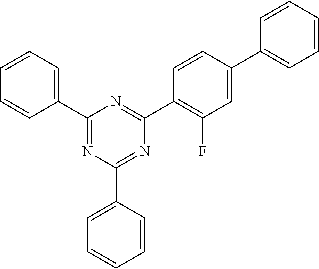 |
| 1-50 | 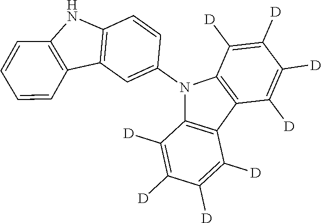 | 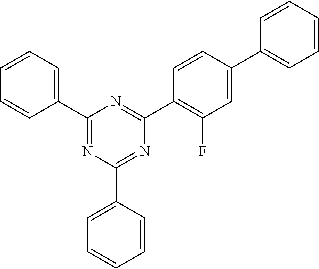 |
| 1-51 | 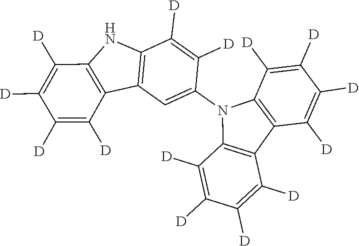 | 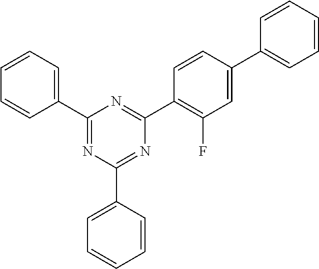 |
| 1-52 | 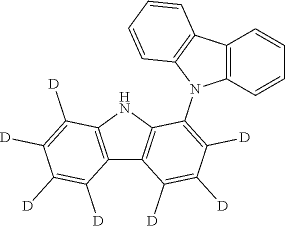 | 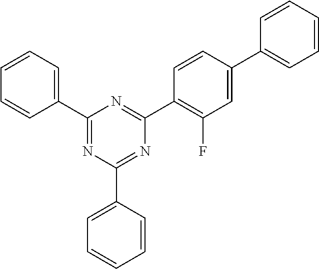 |
| 1-53 | 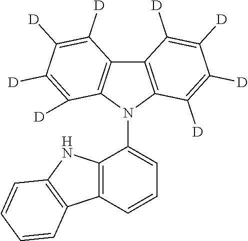 | 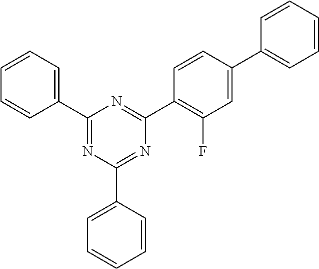 |
| 1-54 | 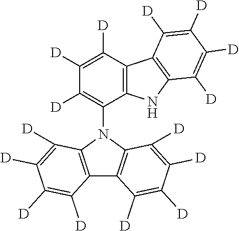 | 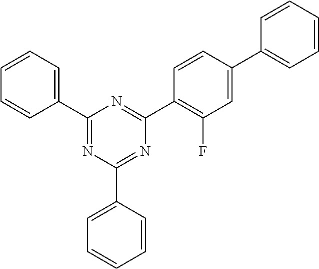 |
| 1-55 | 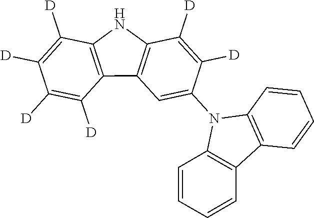 | 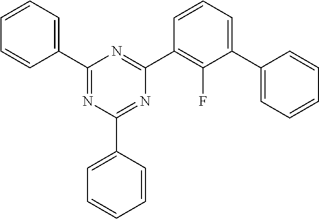 |
| 1-56 | 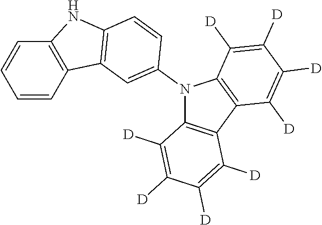 | 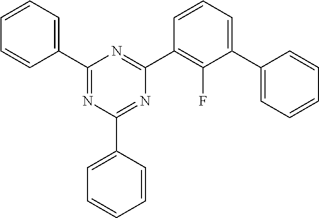 |
| 1-57 | 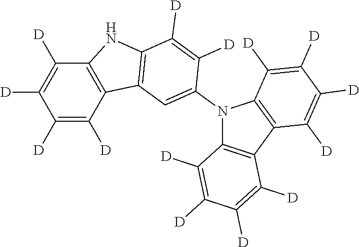 | 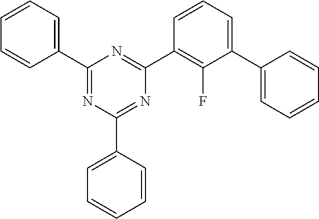 |
| 1-58 | 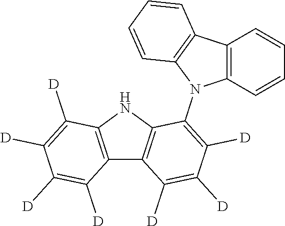 | 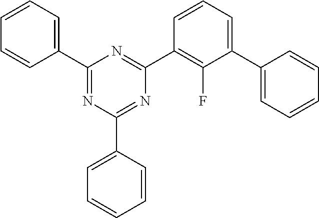 |
| 1-59 | 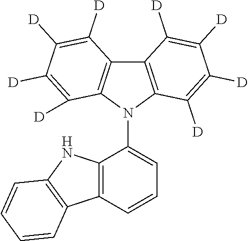 | 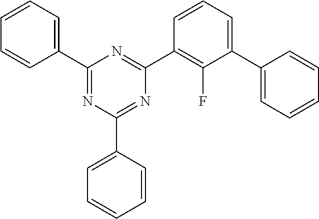 |
| 1-60 | 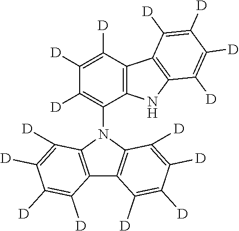 | 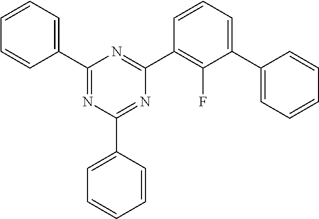 |
| 1-79 | 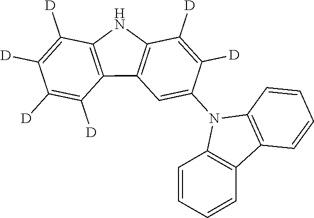 | 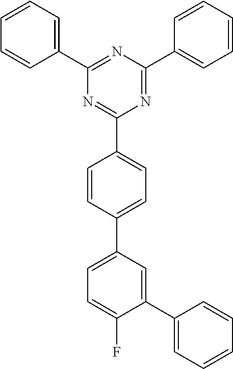 |
| 1-80 | 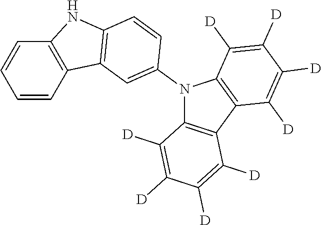 | 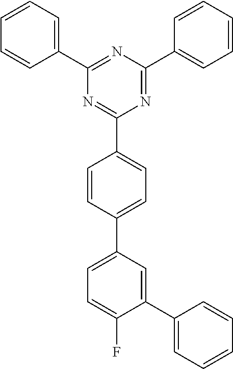 |
| 1-81 | 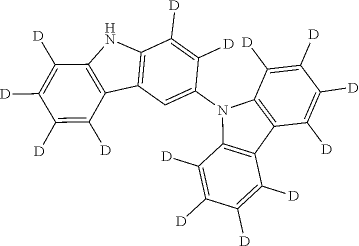 | 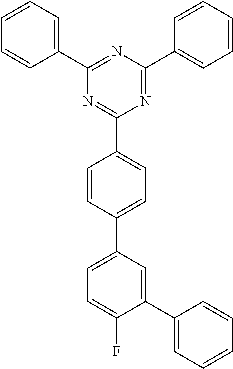 |
| 1-82 | 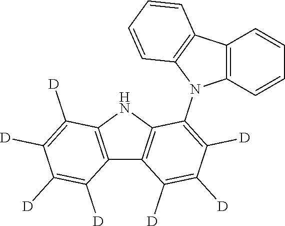 | 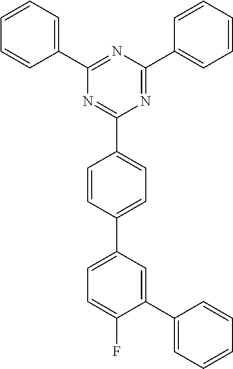 |
| 1-83 | 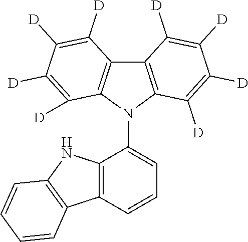 | 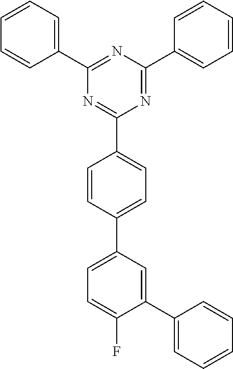 |
| 1-84 | 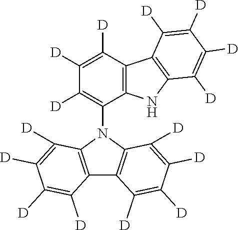 | 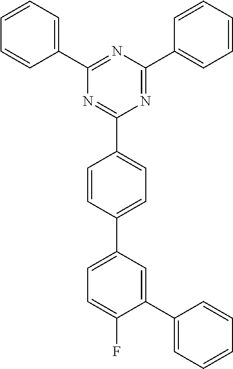 |
| 1-110 | 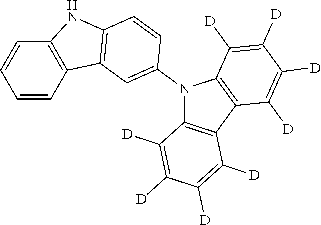 | 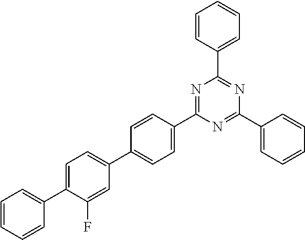 |
| 1-111 | 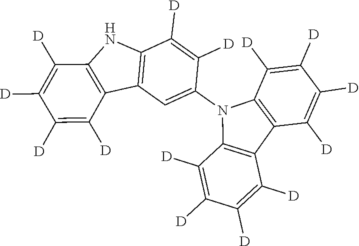 | 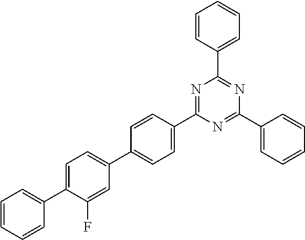 |
| 1-112 | 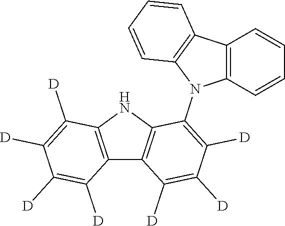 | 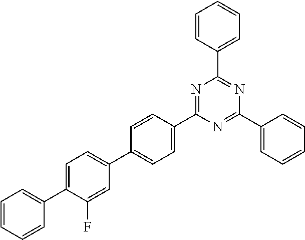 |
| 1-113 | 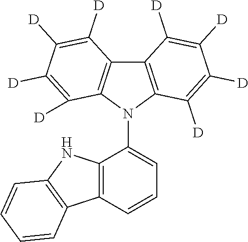 | 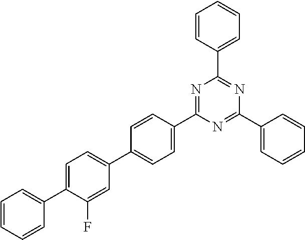 |
| 1-114 | 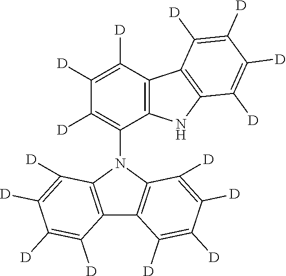 | 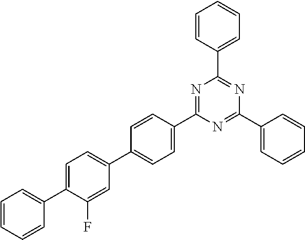 |
| 1-115 | 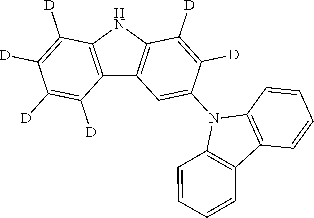 | 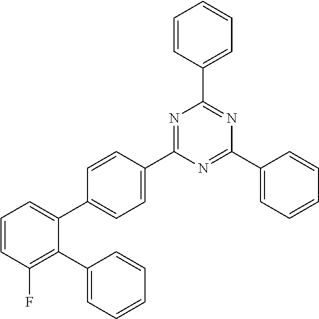 |
| 1-135 | 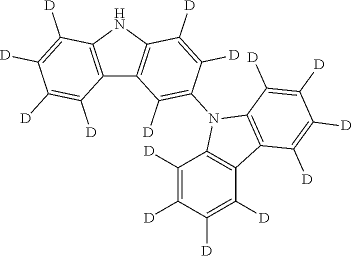 | 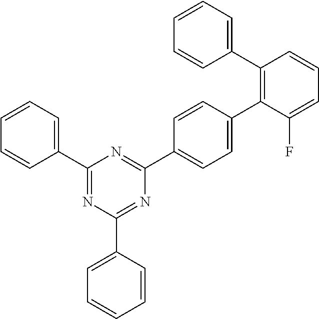 |
| 1-138 | 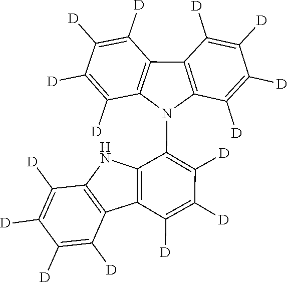 | 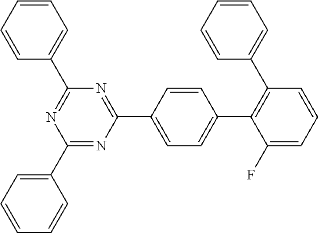 |
| 1-147 | 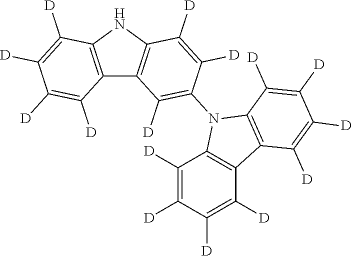 | 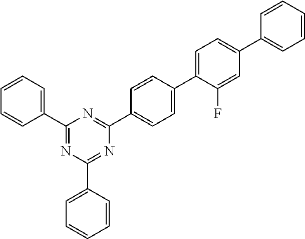 |
| 1-150 | 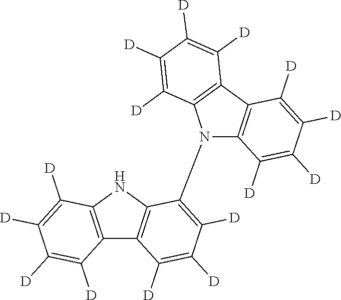 | 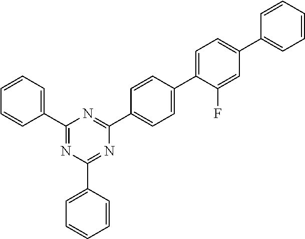 |
| 1-213 | 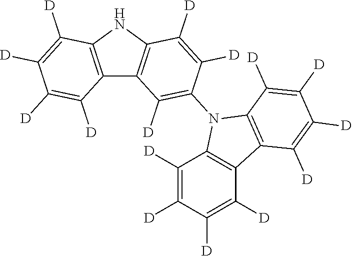 | 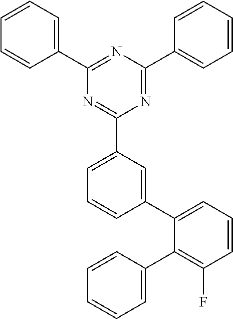 |
| 1-216 | 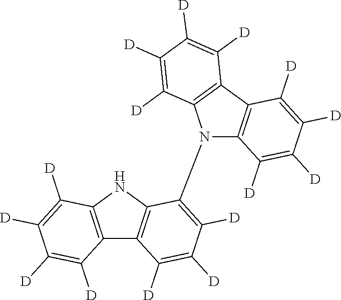 | 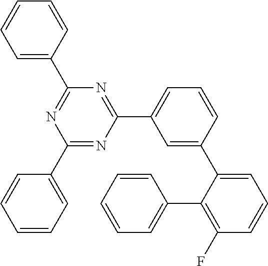 |
| 1-243 | 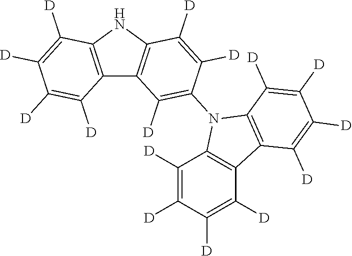 | 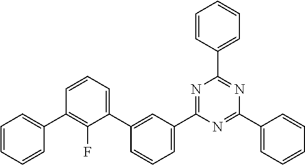 |
| 1-339 | 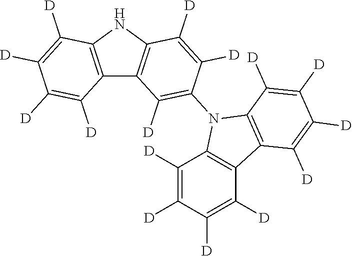 | 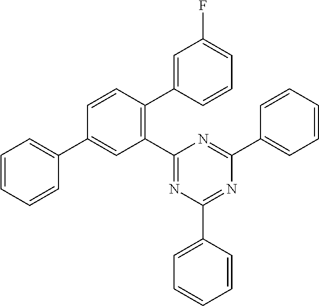 |
| 1-342 | 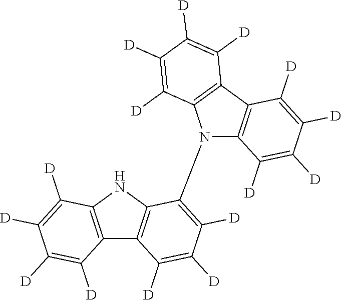 | 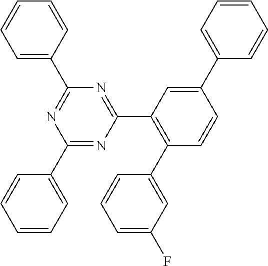 |
| 1-387 | 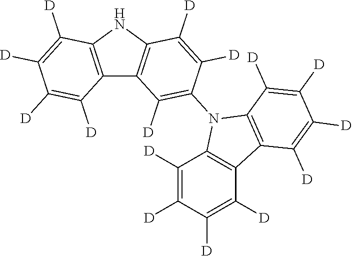 | 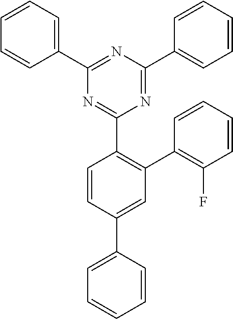 |
| 1-414 | 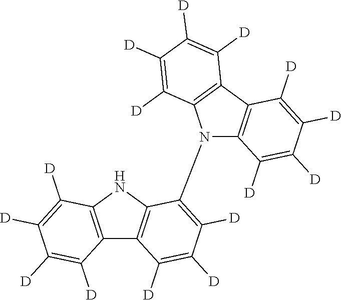 | 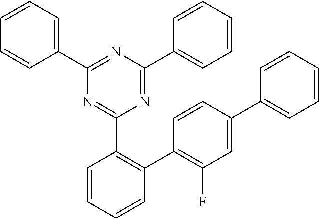 |
| 1-421 | 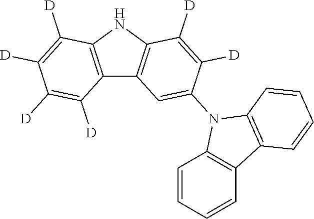 | 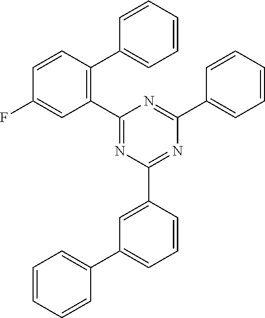 |
| 1-424 | 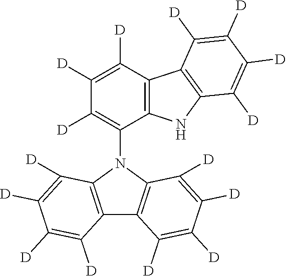 | 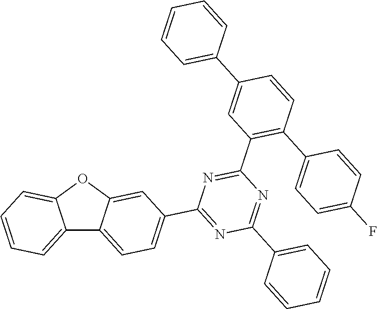 |
| 1-425 | 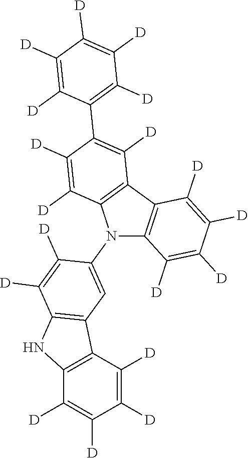 | 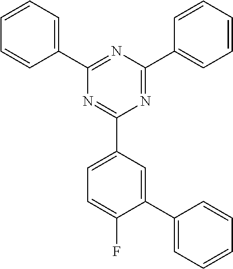 |
| 1-429 | 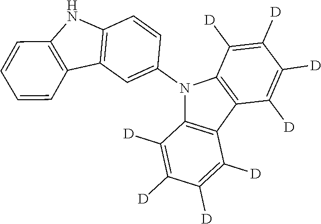 | 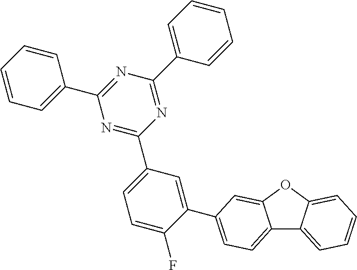 |
| 1-430 | 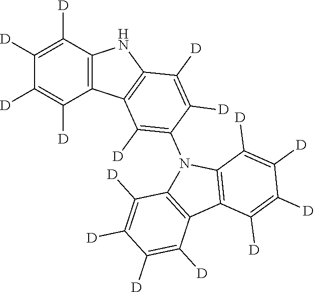 | 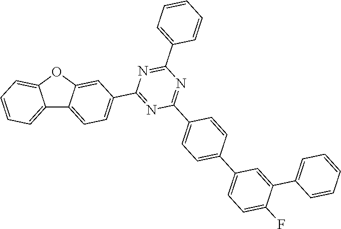 |
| 1-437 | 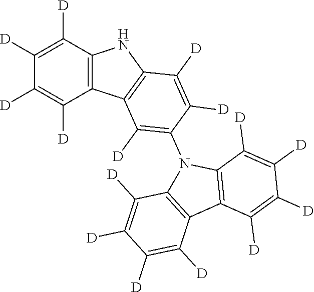 | 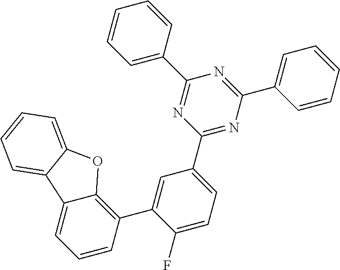 |
| 1-439 | 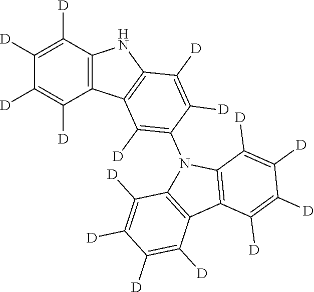 | 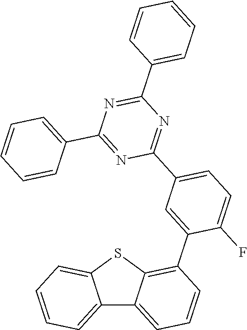 |
| 1-441 | 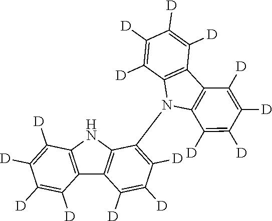 | 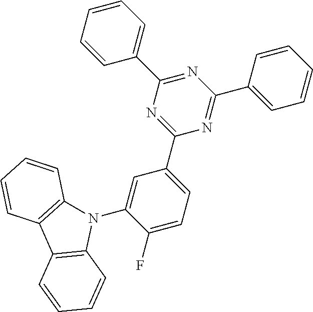 |
| 1-448 | 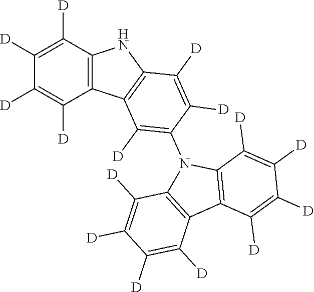 | 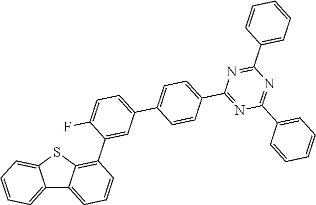 |
| 1-453 | 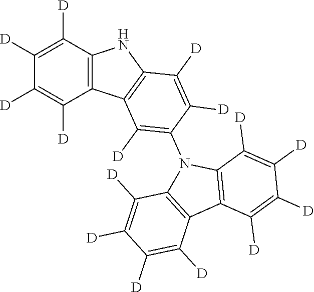 | 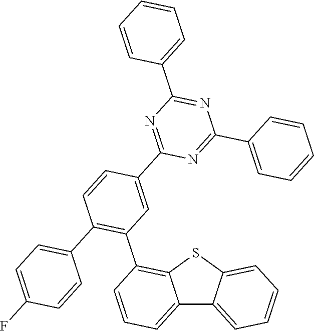 |
| 1-463 | 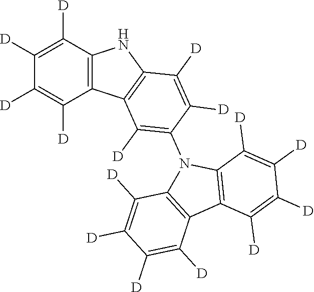 | 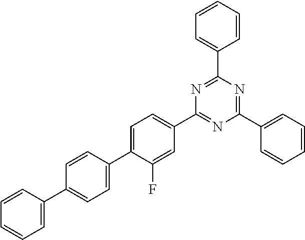 |

| com- | Product | Yield |
|---|---|---|
| pound | C | (%) |
| 1-1 | 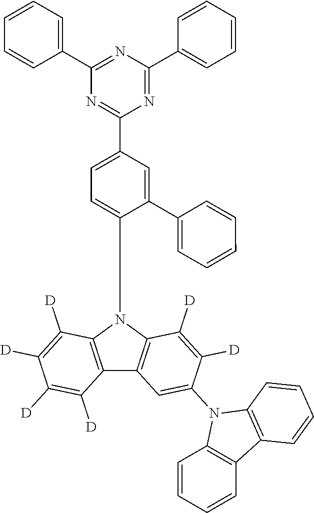 | 75 |
| 1-2 | 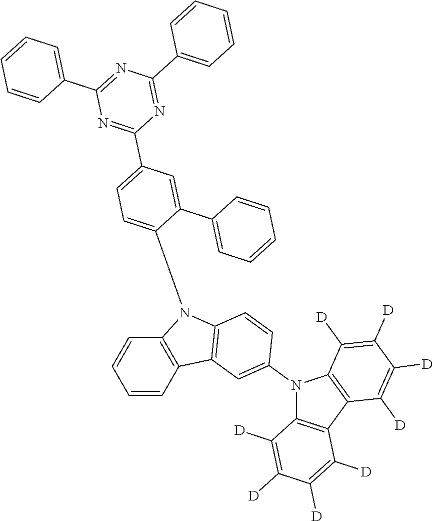 | 76 |
| 1-3 | 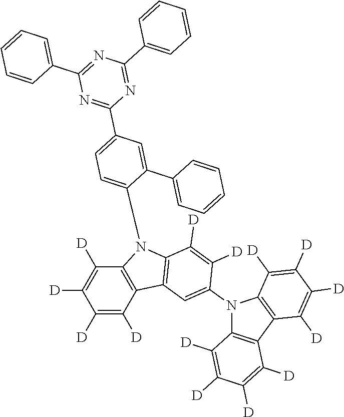 | 72 |
| 1-4 | 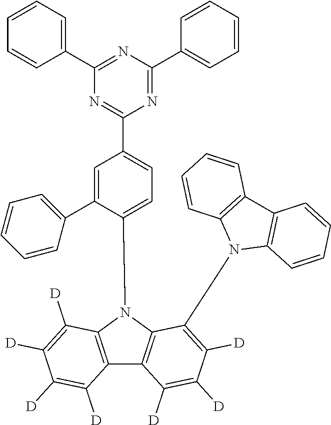 | 72 |
| 1-5 | 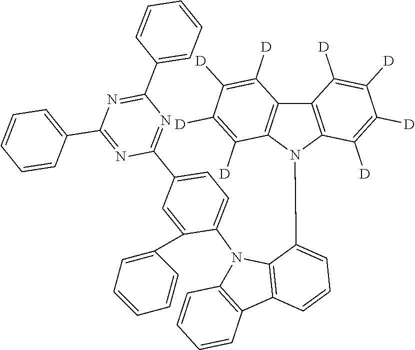 | 76 |
| 1-6 | 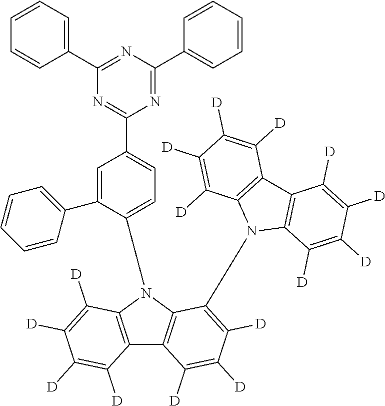 | 72 |
| 1-7 | 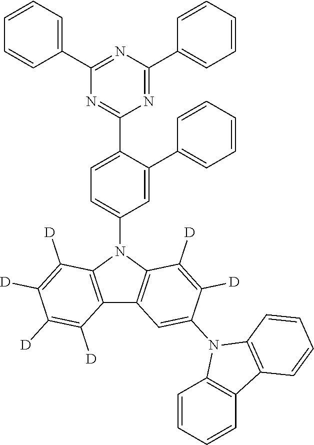 | 71 |
| 1-8 | 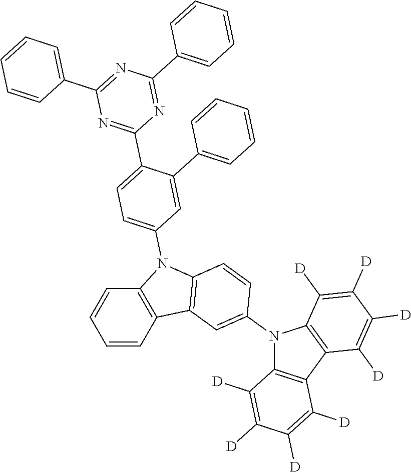 | 78 |
| 1-9 | 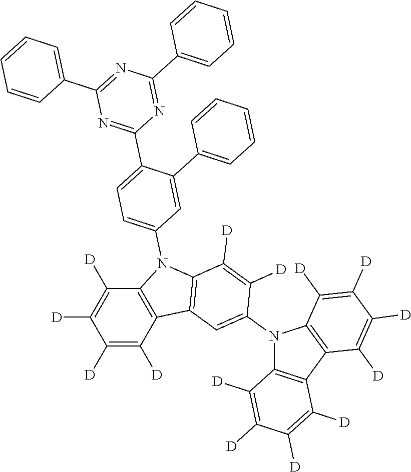 | 70 |
| 1-10 | 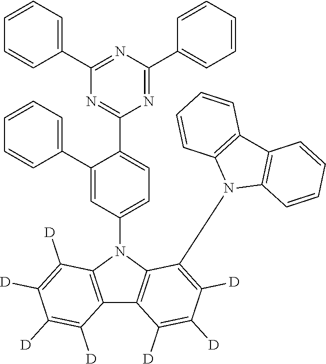 | 74 |
| 1-11 | 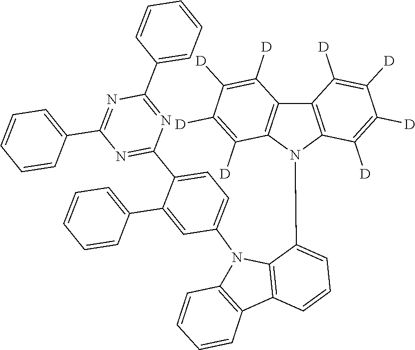 | 78 |
| 1-12 | 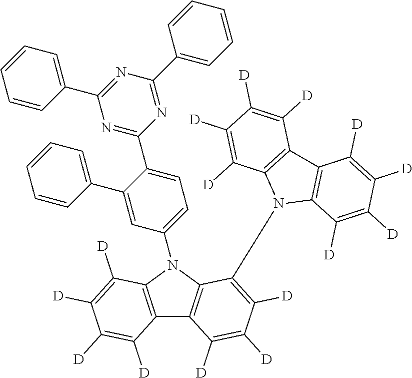 | 70 |
| 1-25 | 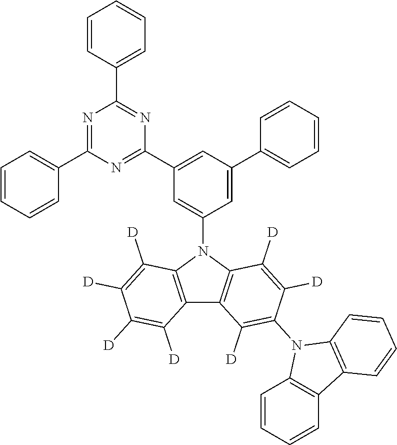 | 80 |
| 1-26 | 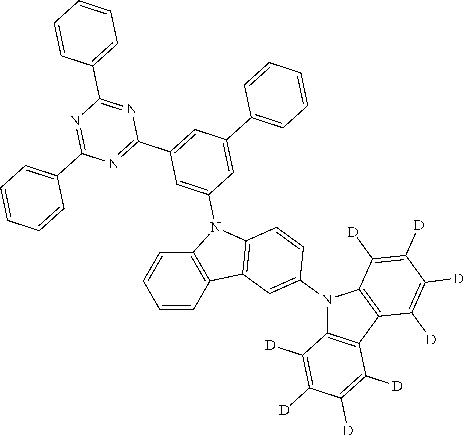 | 73 |
| 1-27 | 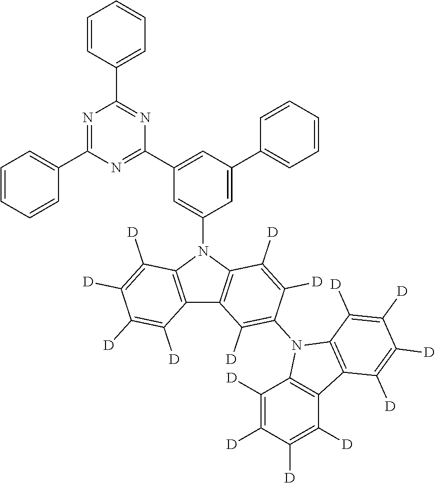 | 76 |
| 1-28 | 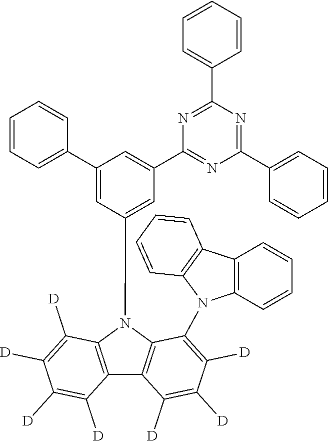 | 71 |
| 1-29 | 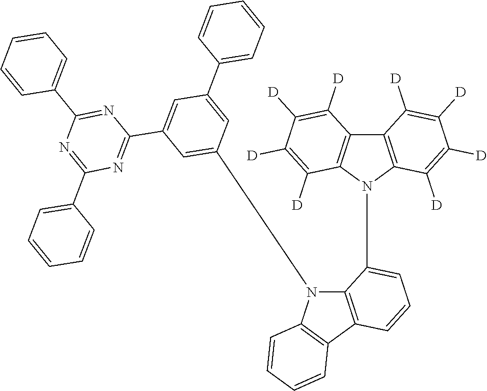 | 78 |
| 1-30 | 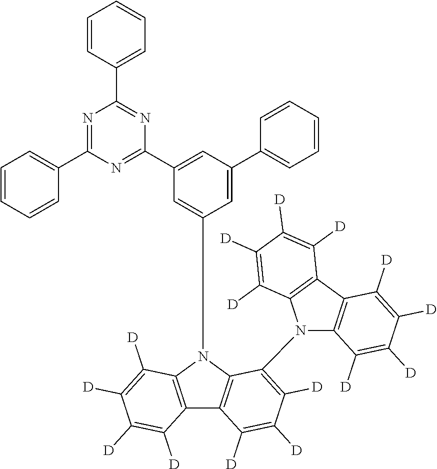 | 70 |
| 1-49 | 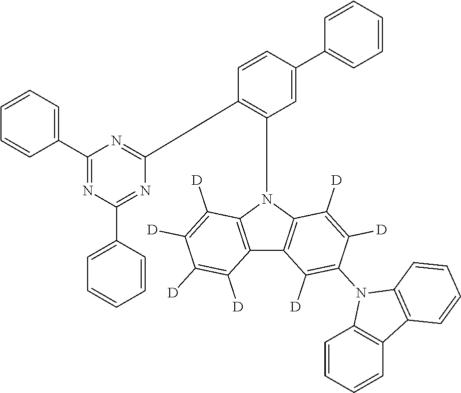 | 72 |
| 1-50 | 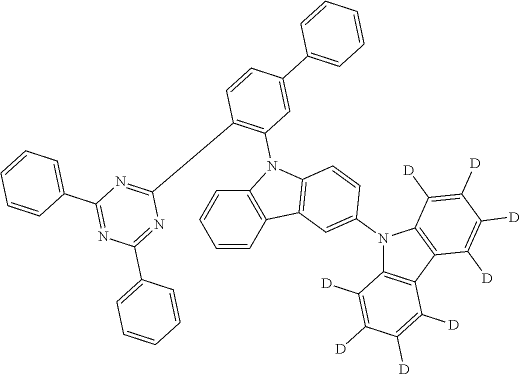 | 79 |
| 1-51 | 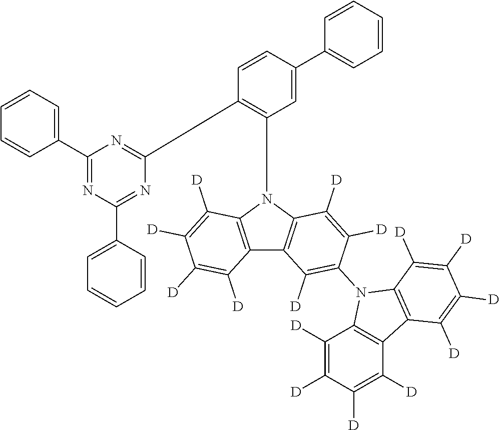 | 79 |
| 1-52 | 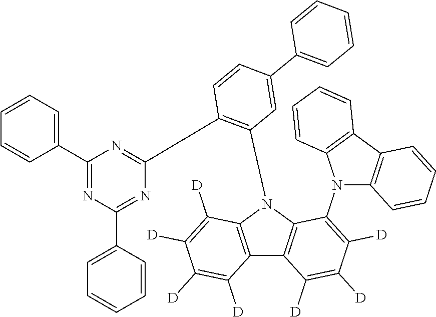 | 77 |
| 1-53 | 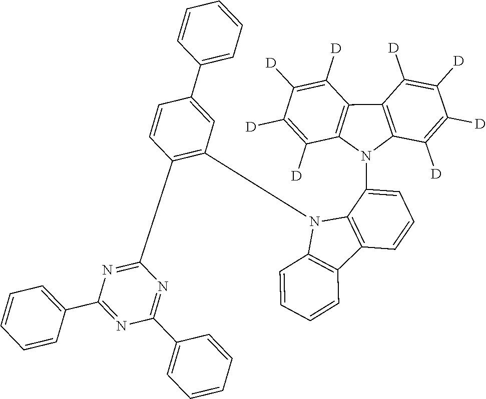 | 71 |
| 1-54 | 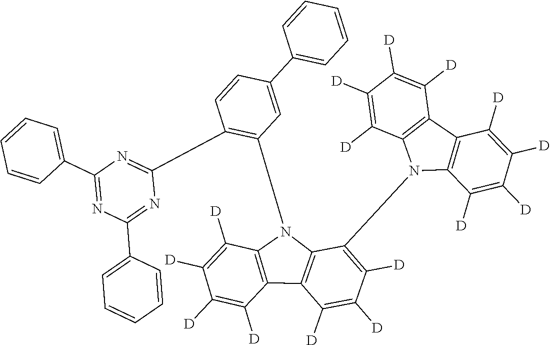 | 80 |
| 1-55 | 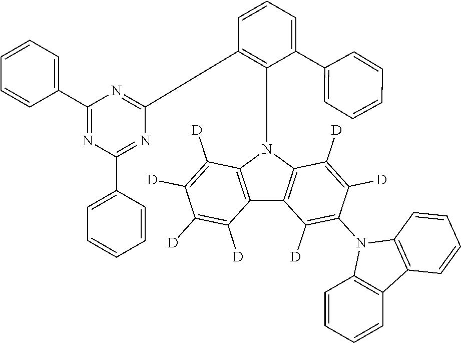 | 77 |
| 1-56 | 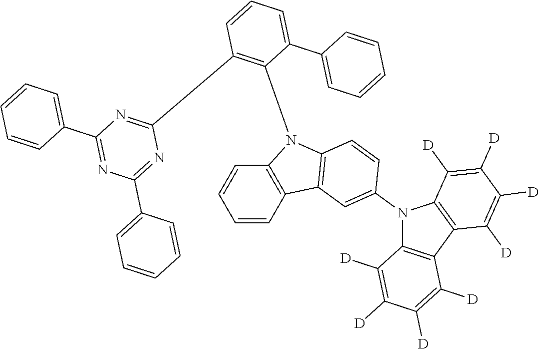 | 76 |
| 1-57 | 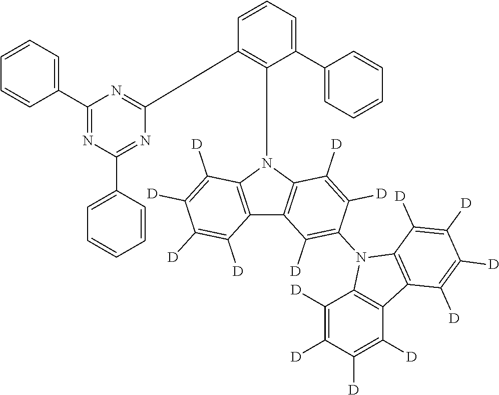 | 71 |
| 1-58 | 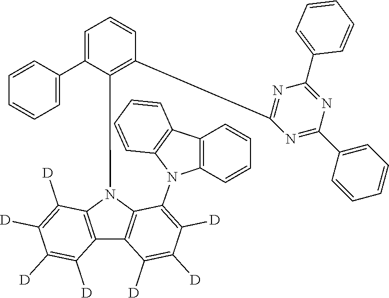 | 75 |
| 1-59 | 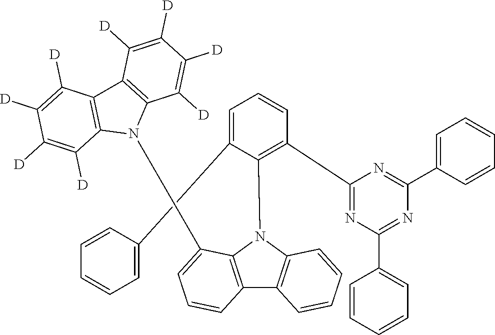 | 79 |
| 1-60 | 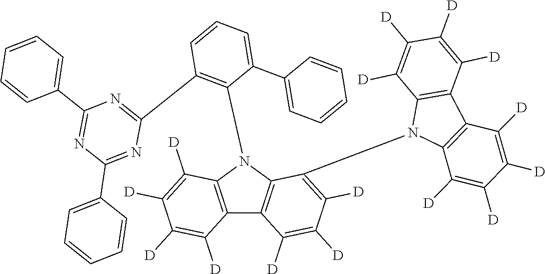 | 73 |
| 1-79 | 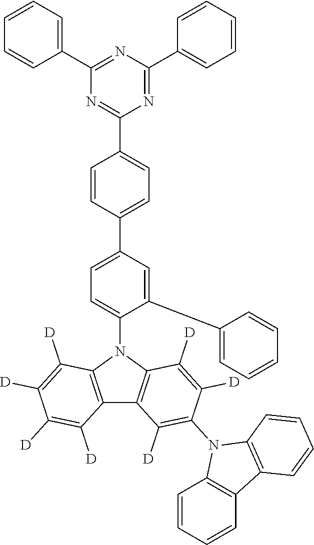 | 70 |
| 1-80 | 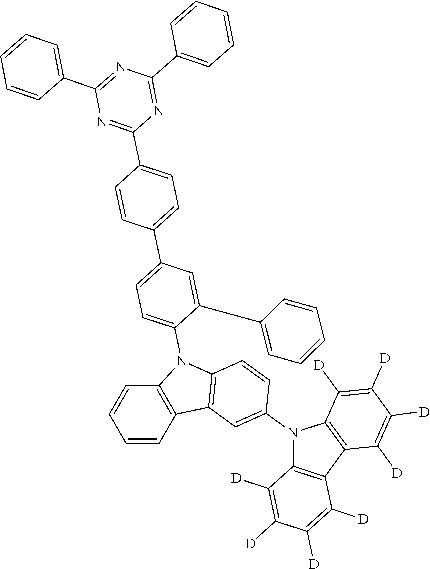 | 80 |
| 1-81 | 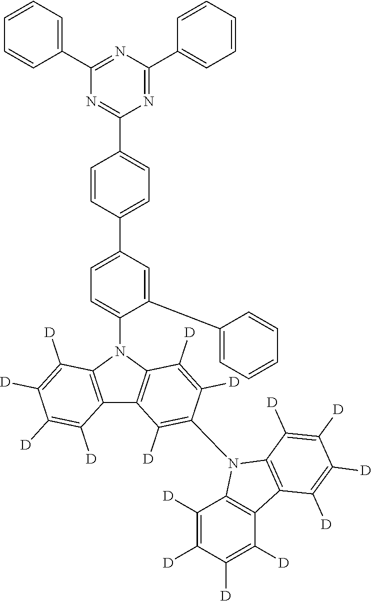 | 78 |
| 1-82 | 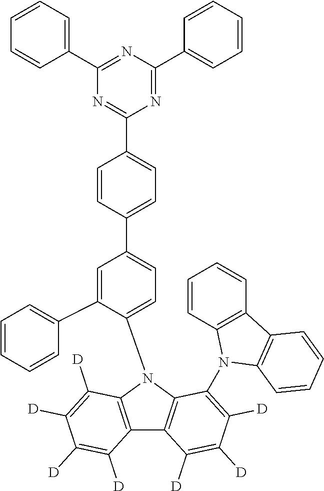 | 73 |
| 1-83 | 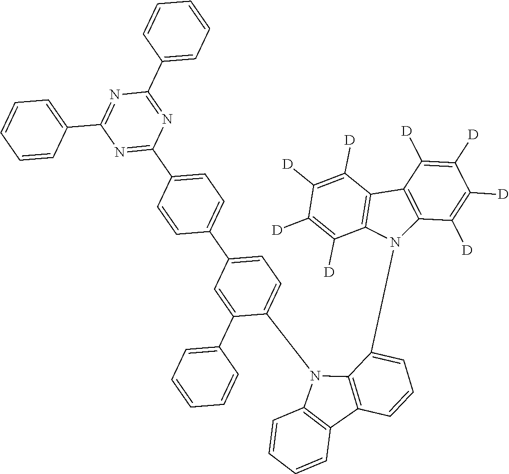 | 80 |
| 1-84 | 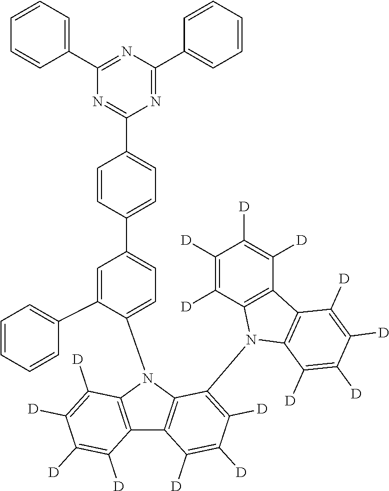 | 72 |
| 1-110 | 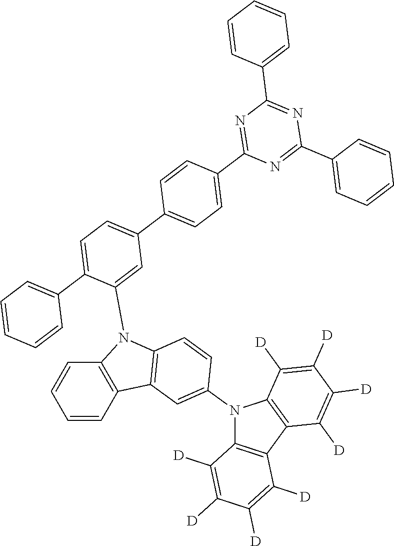 | 80 |
| 1-111 | 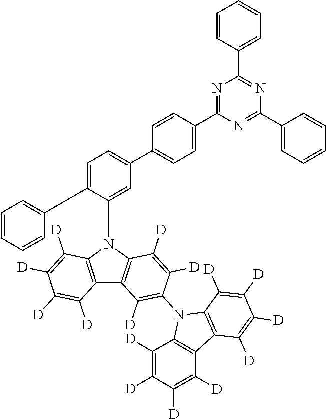 | 80 |
| 1-112 | 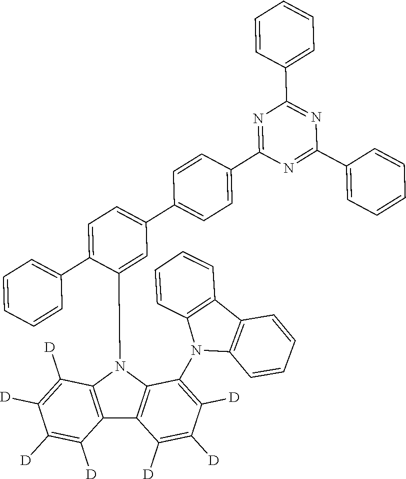 | 74 |
| 1-113 | 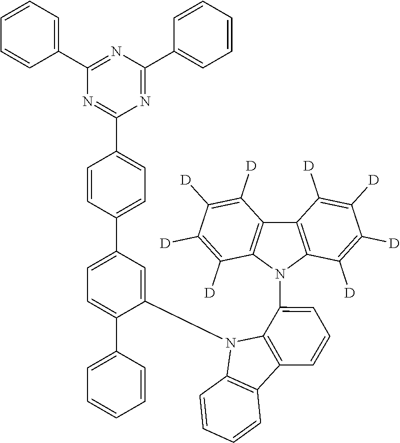 | 71 |
| 1-114 | 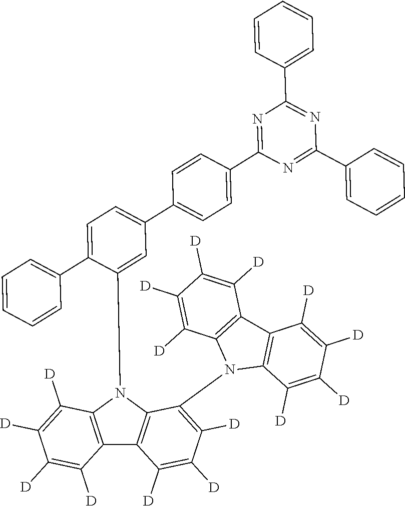 | 72 |
| 1-115 | 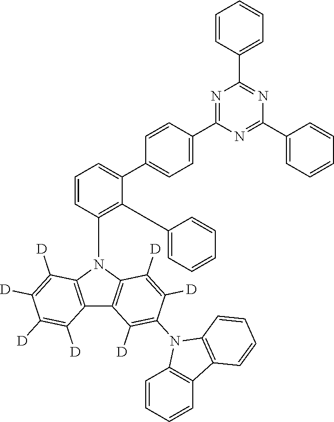 | 79 |
| 1-135 | 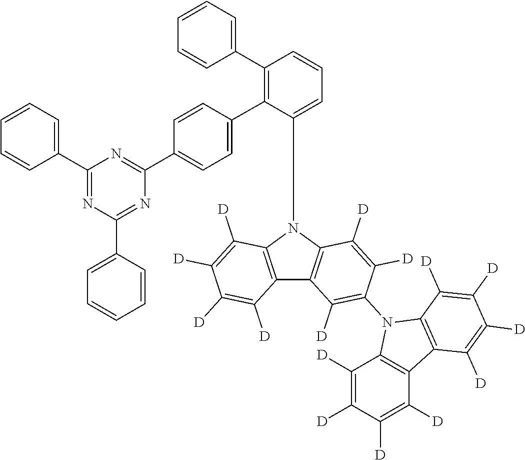 | 71 |
| 1-138 | 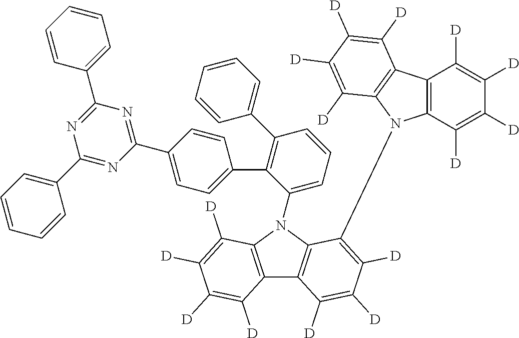 | 69 |
| 1-147 | 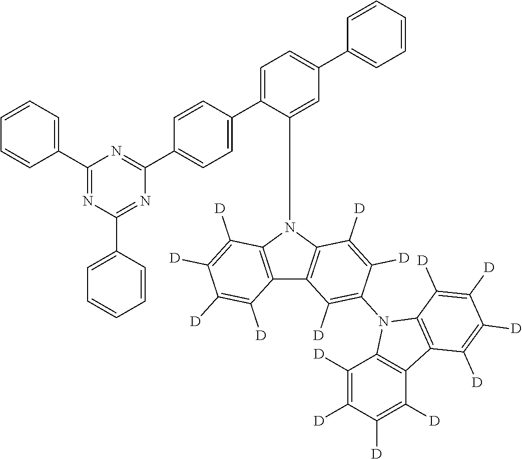 | 71 |
| 1-150 | 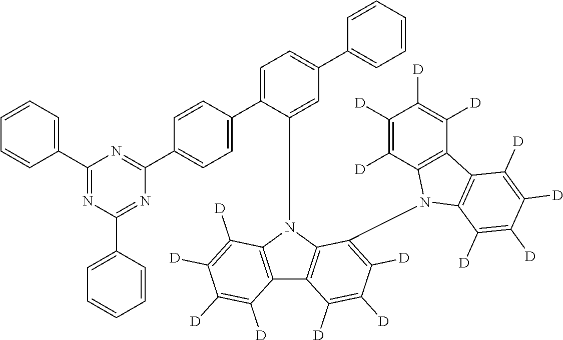 | 70 |
| 1-213 | 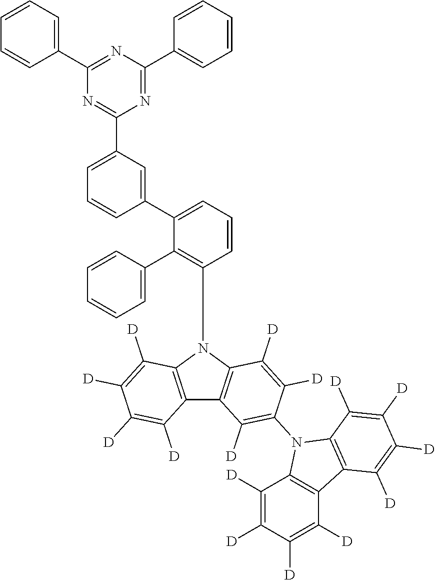 | 68 |
| 1-216 | 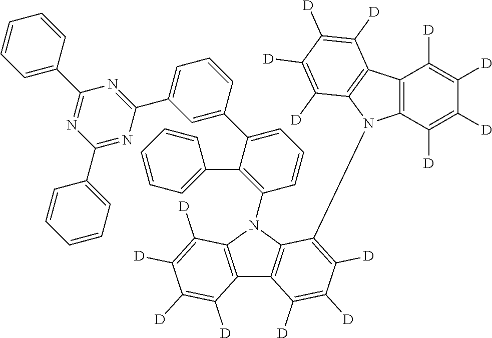 | 70 |
| 1-243 | 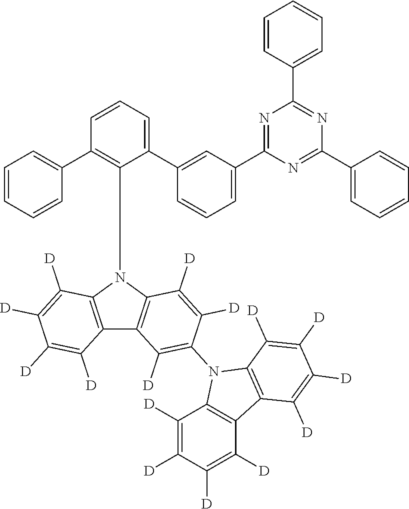 | 71 |
| 1-339 | 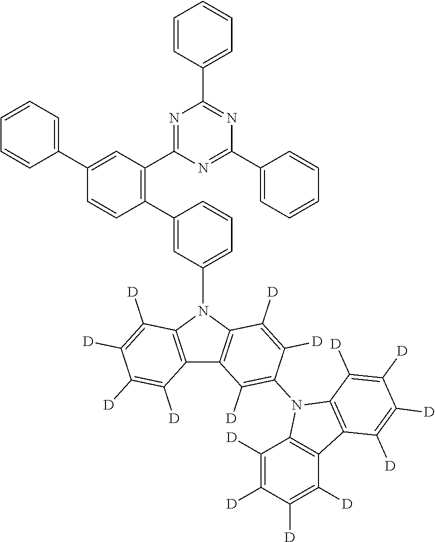 | 72 |
| 1-342 | 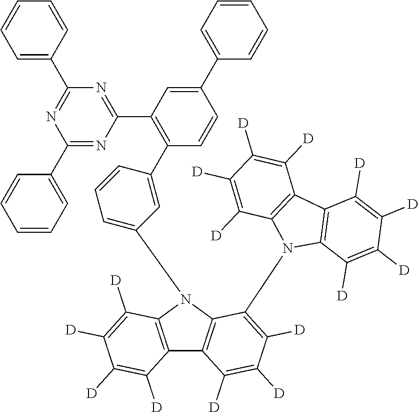 | 71 |
| 1-387 | 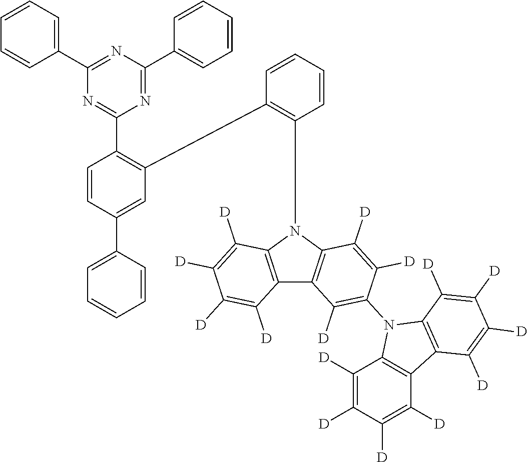 | 70 |
| 1-414 | 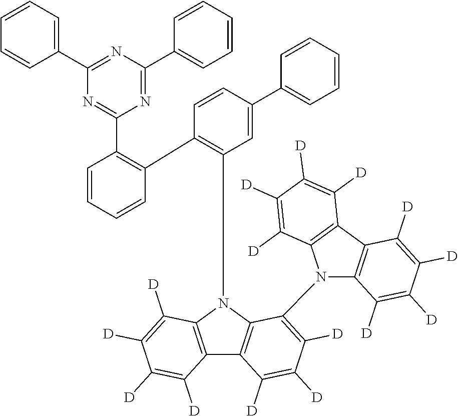 | 73 |
| 1-421 | 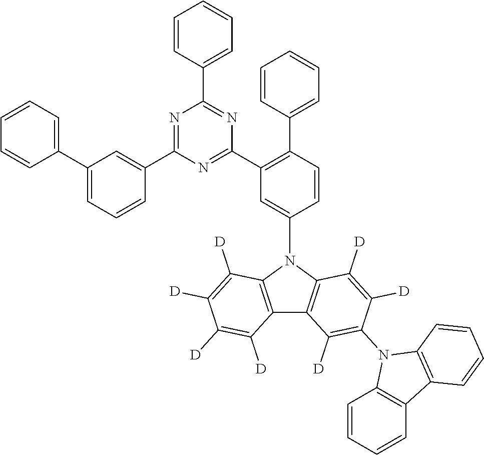 | 73 |
| 1-424 | 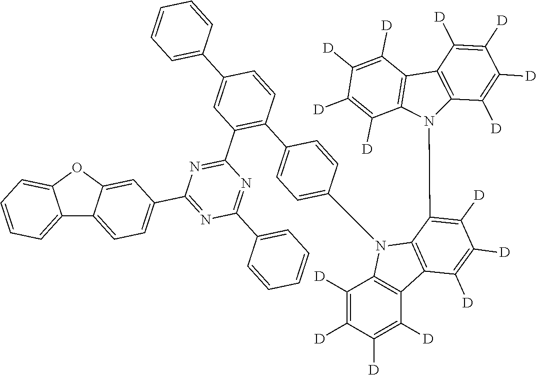 | 78 |
| 1-425 | 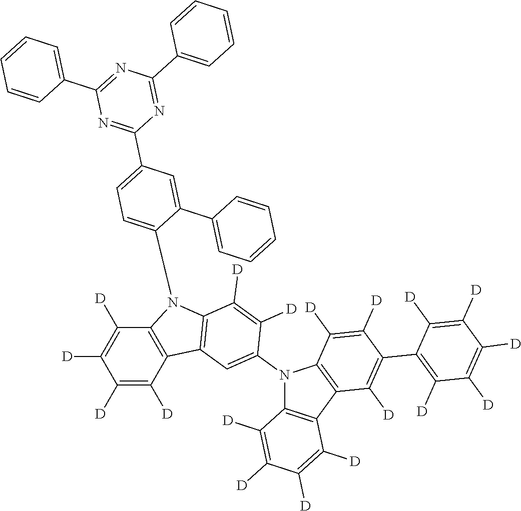 | 75 |
| 1-429 | 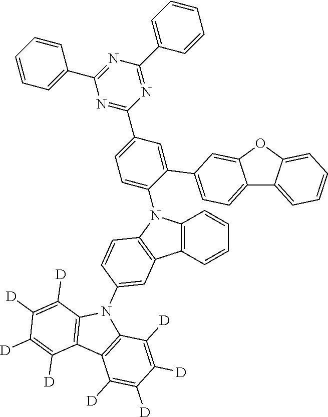 | 71 |
| 1-430 | 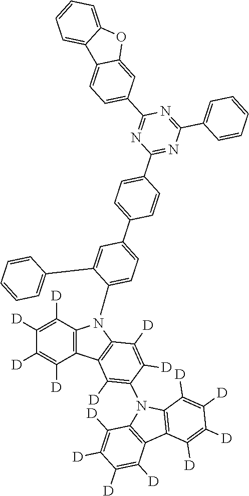 | 71 |
| 1-437 | 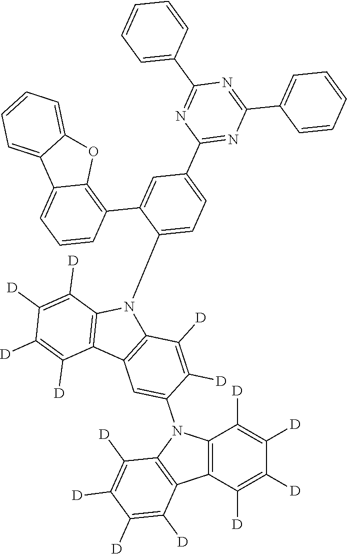 | 72 |
| 1-439 | 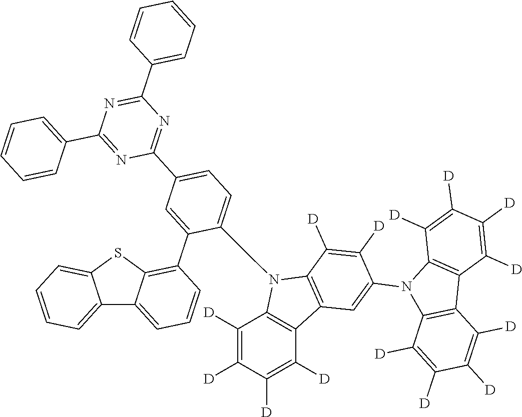 | 79 |
| 1-441 | 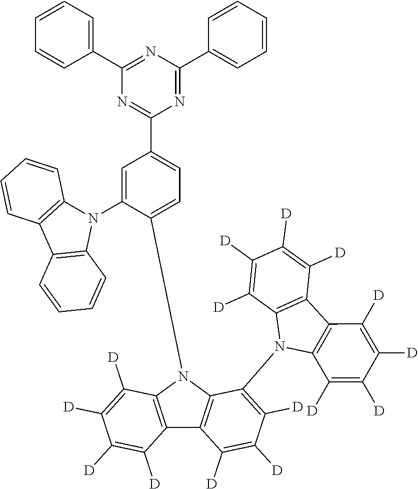 | 69 |
| 1-448 | 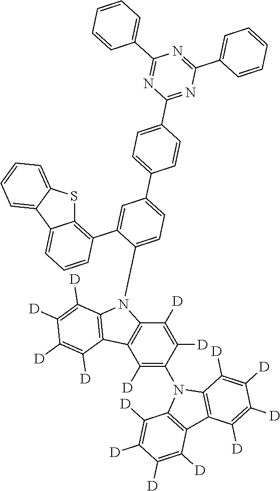 | 71 |
| 1-453 | 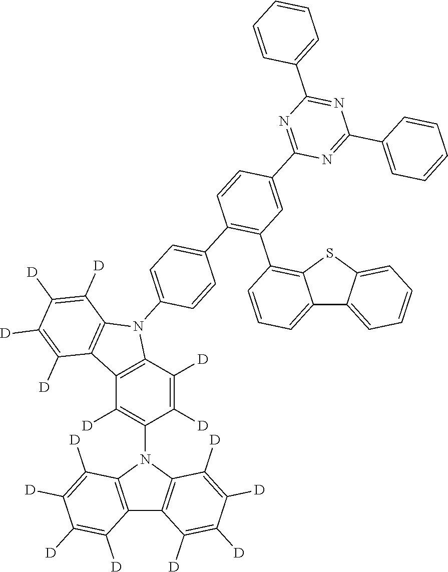 | 70 |
| 1-463 | 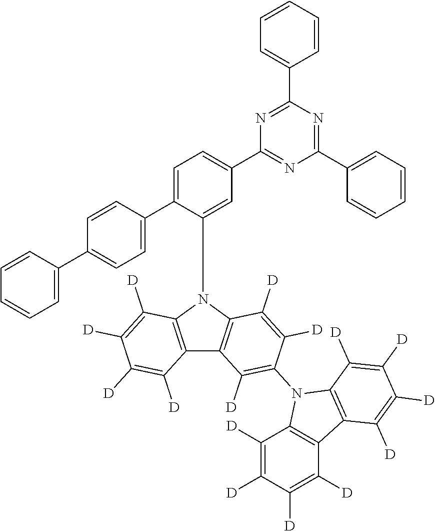 | 71 |

### [Preparation Example 2] Preparation of Compound 2-1 [C]

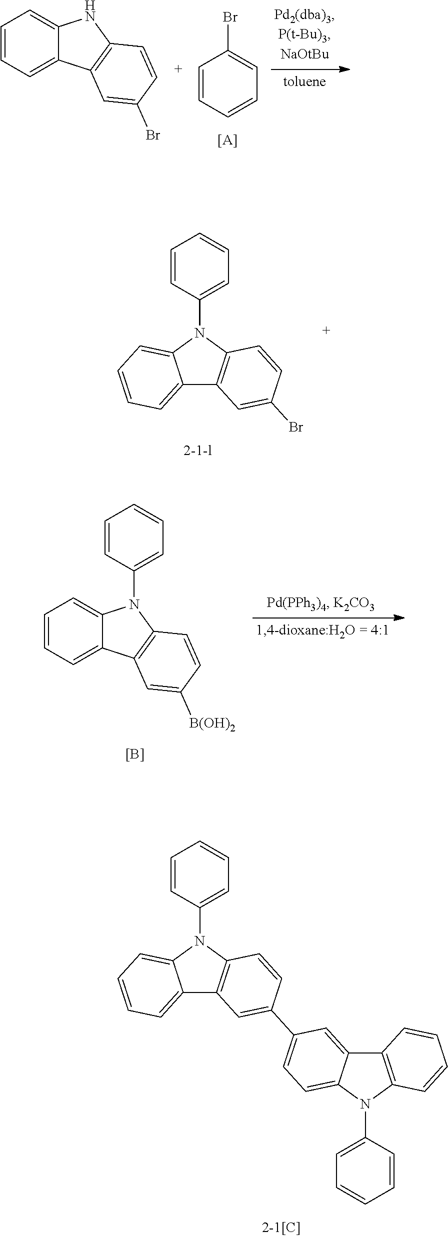

### Preparation of Compound 2-1-i

3-bromo-9H-carbazole (10 g, 49.59 mmol), 2-bromobenzene-1-ylium (24.2 g, 148.77 mmol) [A], Pd₂(dba)₃ (2.27 g, 2.48 mmol), P(t-Bu)₃ (2.42 mL, 9.92 mmol), and NaOtBu (9.53 g, 99.18 mmol) were added to a one-neck round bottom flask, and then toluene (100 mL) was added thereto, followed by heating at 135° C. for 15 hours.

When the reaction was completed, extraction was performed with MC and H₂O, followed by column purification to obtain Compound 2-1-i (14 g, 98%).

### Preparation of Compound 2-1[C]

2-[C]-1-i (14 g, 43.4 mmol), (9-phenyl-9H-carbazol-3-yl)boronic acid (14.9 g, 52 mmol) [B], Pd(PPh₃)₄ (2.5 g, 2.17 mmol), and K₂CO₃ (17.9 g, 130 mmol) were added to a one-neck round bottom flask, and then a mixture of 1,4-dioxane/water (140 ml/35 ml) was heated at 120° C. for 4 hours.

After the reaction was terminated, the temperature was lowered to room temperature, and the generated solid was sequentially washed with distilled water and MeOH to obtain the target Compound 2-1[C] (17 g, 80%).

In Preparation Example 2, a target Compound C was prepared in the same manner except that [A] and [B] were respectively replaced with Intermediates A and B of Table 2 below.

TABLE 2

| | Intermediate | Intermediate | Target compound | Yield |
|---|---|---|---|---|
| Compound | A | B | C | (%) |
| 2-2 | 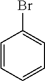 | 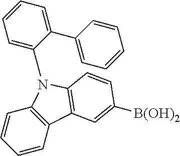 | 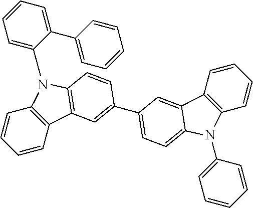 | 80% |
| 2-3 | 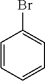 | 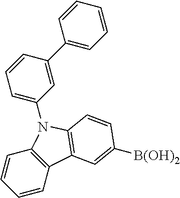 | 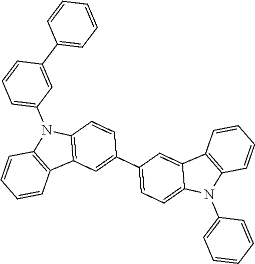 | 76% |
| 2-4 | 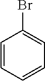 | 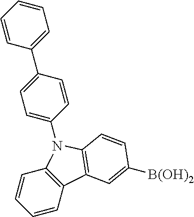 | 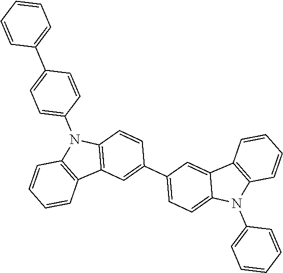 | 69% |
| 2-5 | 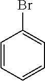 | 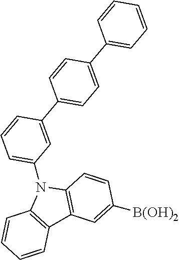 | 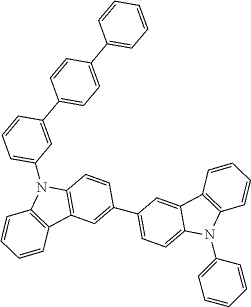 | 68% |
| 2-6 | 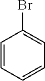 | 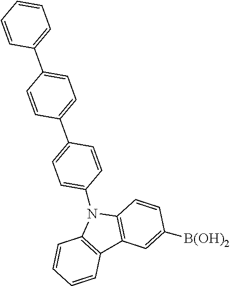 | 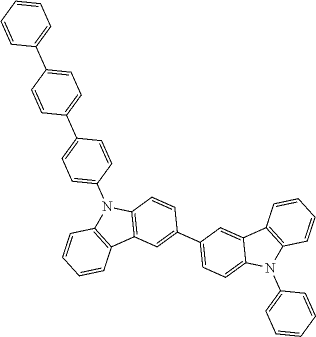 | 70% |
| 2-7 | 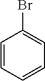 | 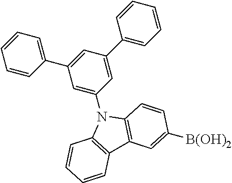 | 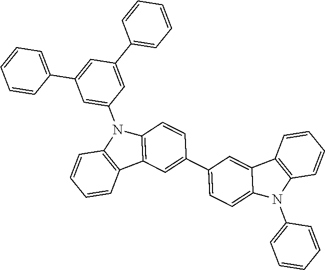 | 73% |
| 2-11 | 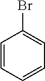 | 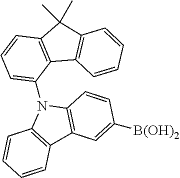 | 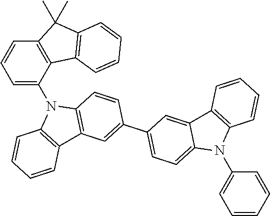 | 69% |
| 2-16 | 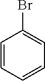 | 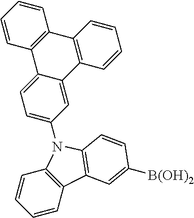 | 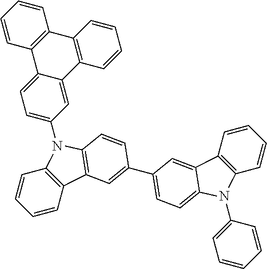 | 70% |
| 2-19 | 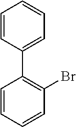 | 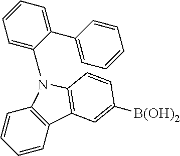 | 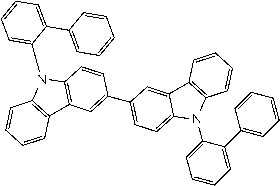 | 76% |
| 2-20 | 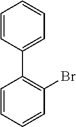 | 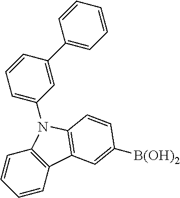 | 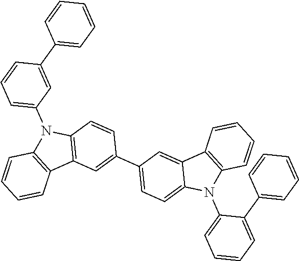 | 84% |
| 2-21 | 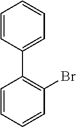 | 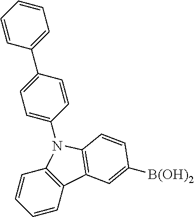 | 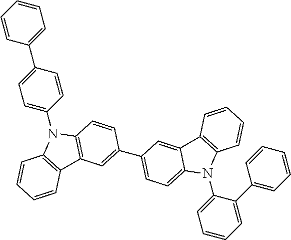 | 80% |
| 2-22 | 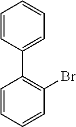 | 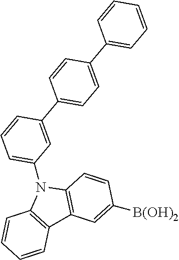 | 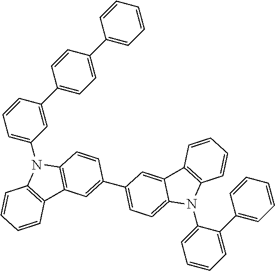 | 83% |
| 2-23 | 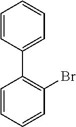 | 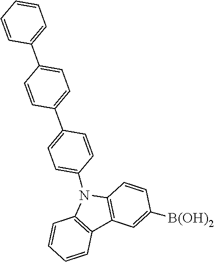 | 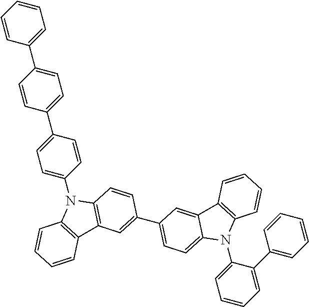 | 70% |
| 2-26 | 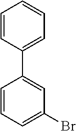 | 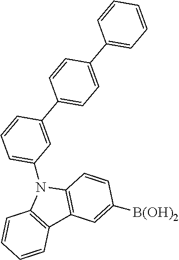 | 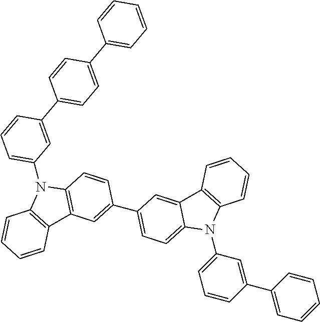 | 73% |
| 2-27 | 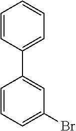 | 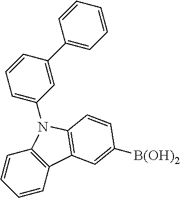 | 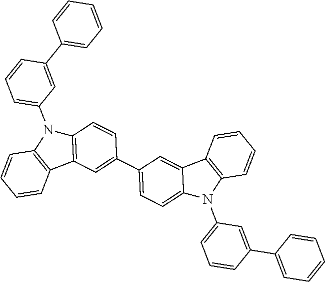 | 84% |
| 2-28 | 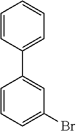 | 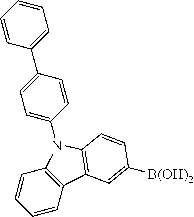 | 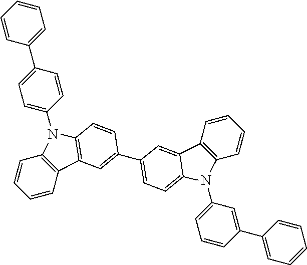 | 74% |
| 2-29 | 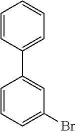 | 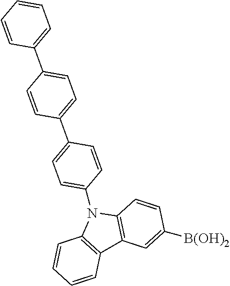 | 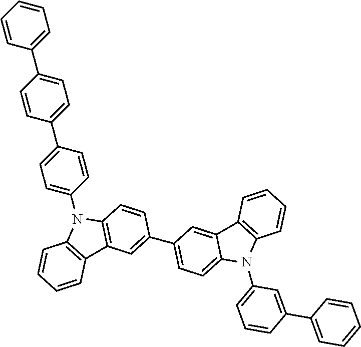 | 69% |
| 2-30 | 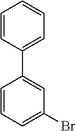 | 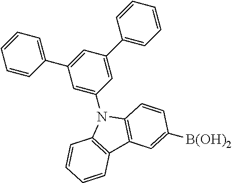 | 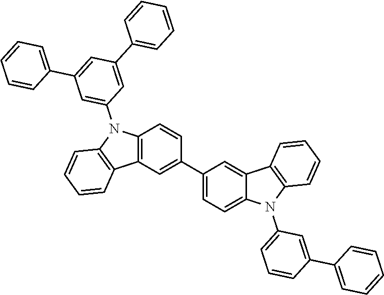 | 76% |
| 2-32 | 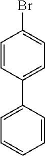 | 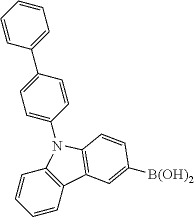 | 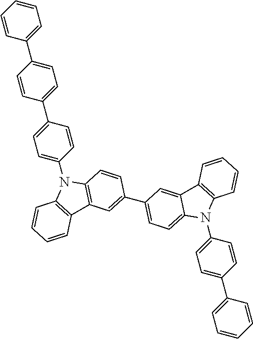 | 75% |
| 2-33 | 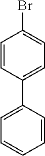 | 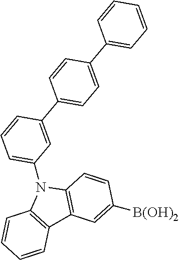 | 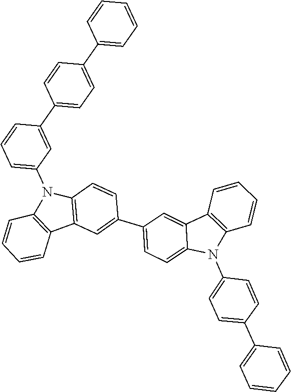 | 72% |
| 2-34 | 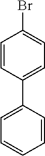 | 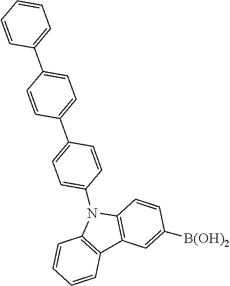 | 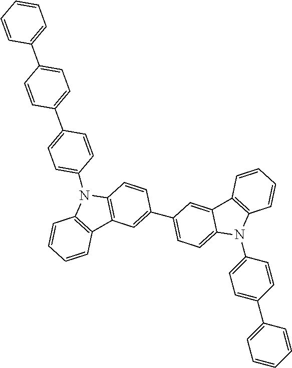 | 84% |
| 2-38 | 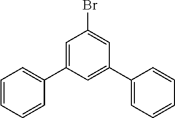 | 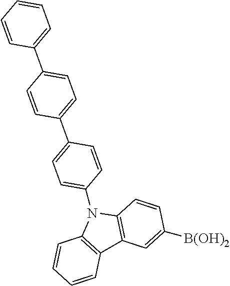 | 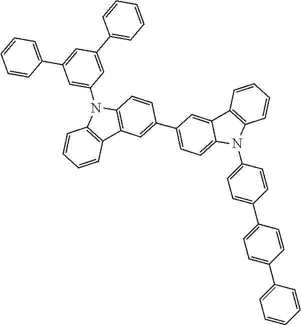 | 74% |
| 2-40 | 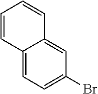 | 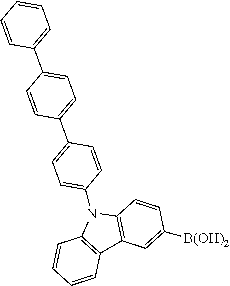 | 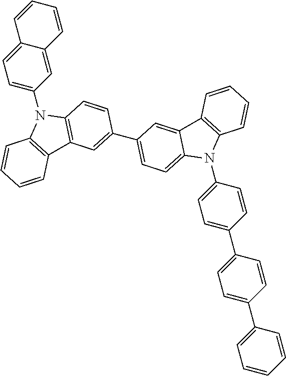 | 84% |
| 2-41 | 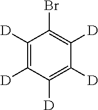 | 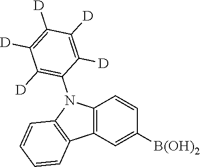 | 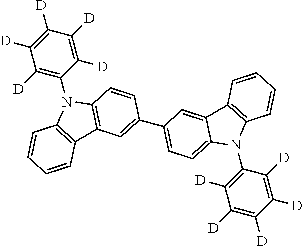 | 70% |
| 2-42 | 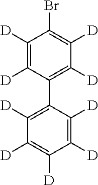 | 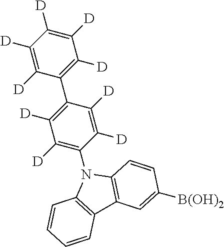 | 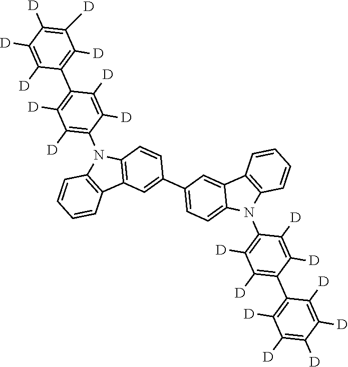 | 84% |
| 2-43 | 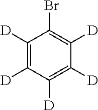 | 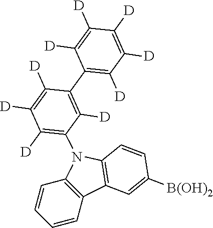 | 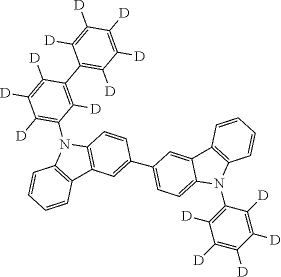 | 81% |
| 2-45 | 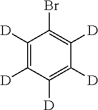 | 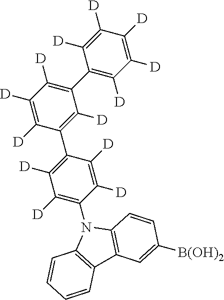 | 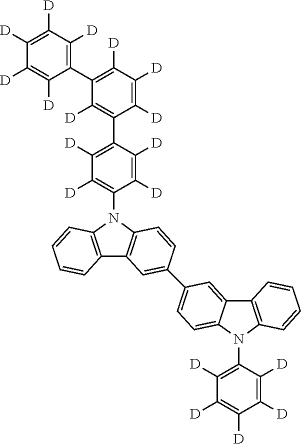 | 68% |
| 2-46 | 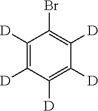 | 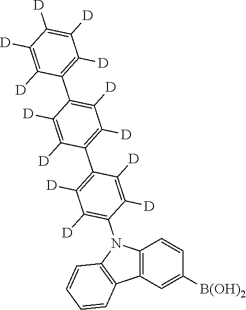 | 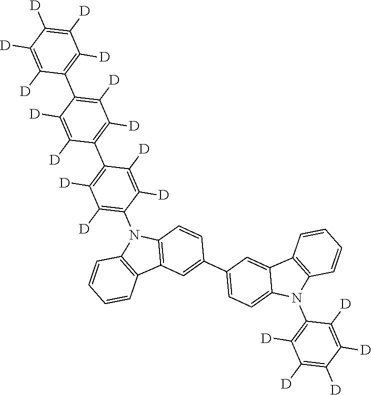 | 81% |
| 2-48 | 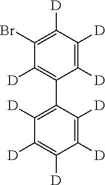 | 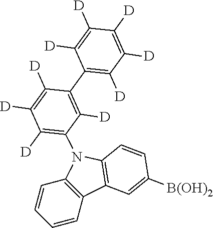 | 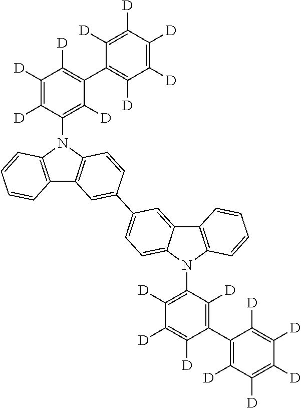 | 70% |
| 2-49 | 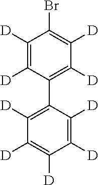 | 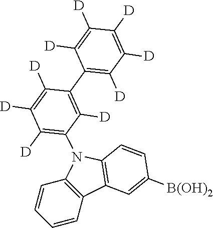 | 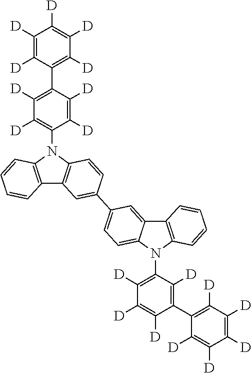 | 71% |
| 2-50 | 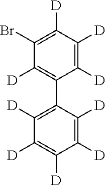 | 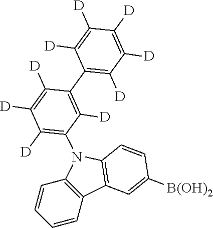 | 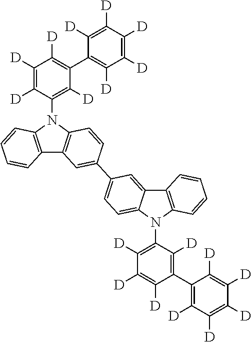 | 74% |
| 2-51 | 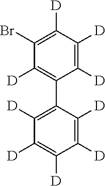 | 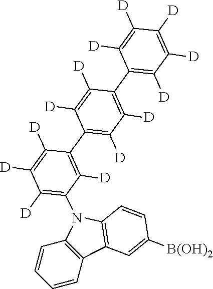 | 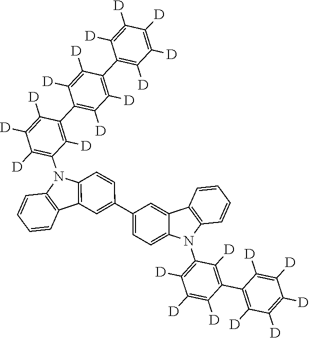 | 80% |
| 2-52 | 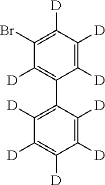 | 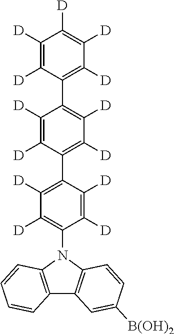 | 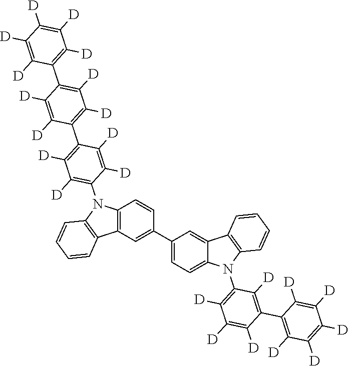 | 84% |
| 2-55 | 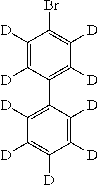 | 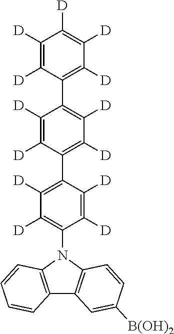 | 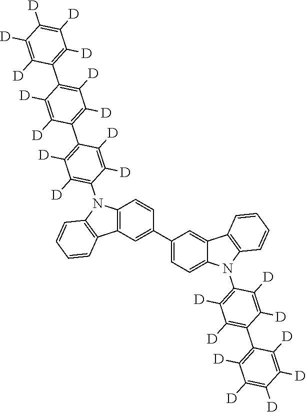 | 76% |
| 2-57 | 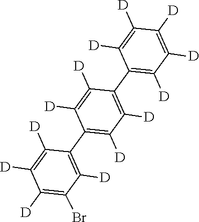 | 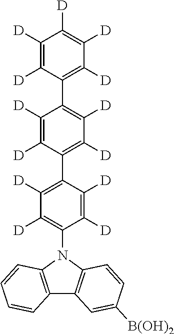 | 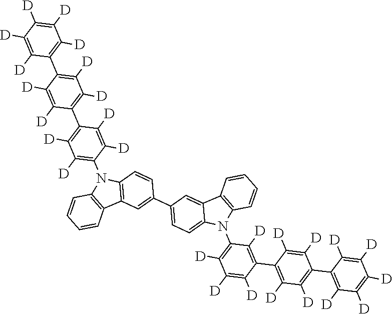 | 68% |
| 2-60 | 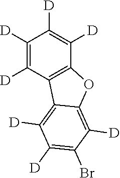 | 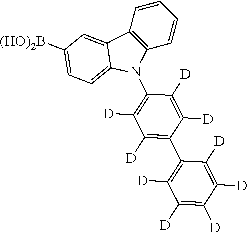 | 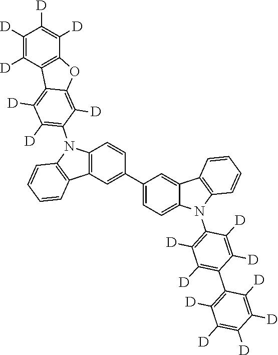 | 80% |

### [Preparation Example 3] Preparation of Compound 2-61[F]

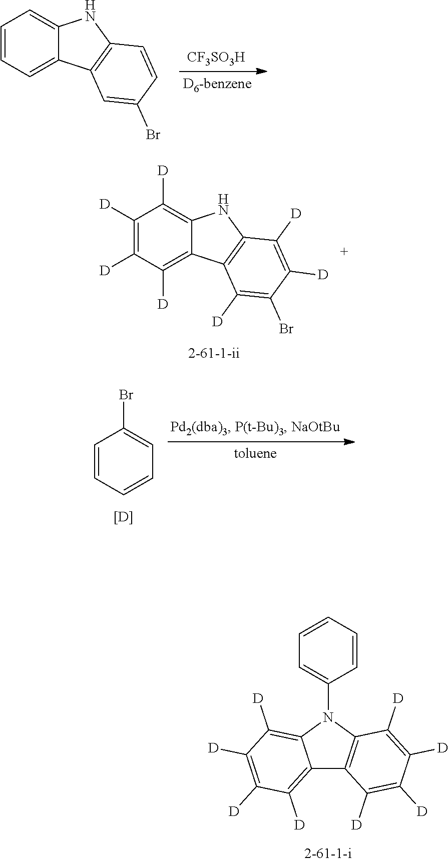

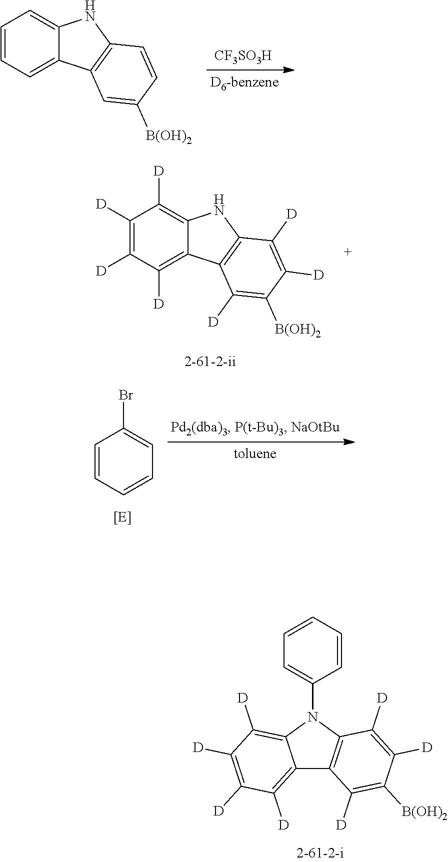

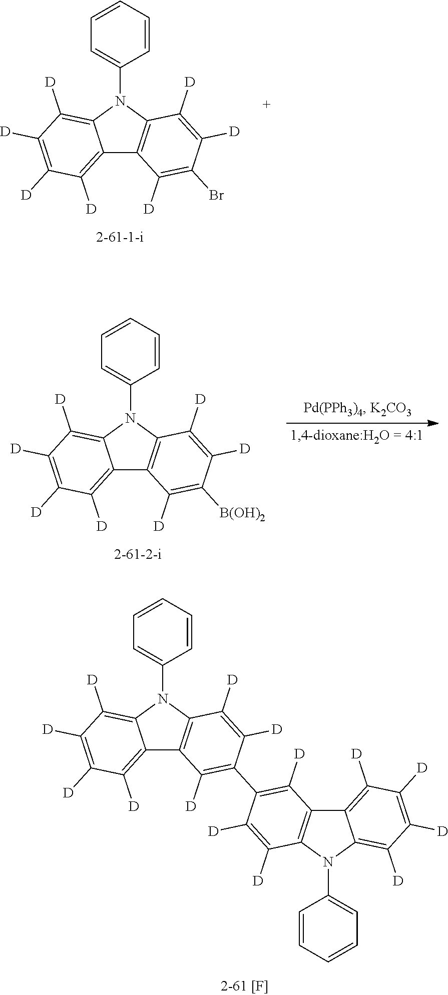 [SMILES: [2H]c1c([2H])c([2H])c2c(c1[2H])c1c([2H])c(Br)c([2H])c([2H])c1n2-c1ccccc1 ; [2H]c1c([2H])c([2H])c2c(c1[2H])c1c([2H])c(B(O)O)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

### Preparation of Compound 2-61-1-ii

3-bromo-9H-carbazole (10 g, 40.23 mmol) and 1000 ml of D₆-benzene were added to a one-neck round bottom flask, and then CF₃SO₃H (170 g, 1075 mmol) was added thereto, followed by stirring at 50° C.

When the reaction was completed, neutralization was performed with D₂O, followed by extraction with MC and an aqueous Na₂CO₃ solution, and then column purification to obtain Compound 2-61-1-ii (10 g, 98%).

### Preparation of Compound 2-61-1-i

2-61-1-ii (10 g, 39.5 mmol), bromobenzene (12.4 g, 79 mmol) [D], Pd₂(dba)₃ (1.81 g, 1.98 mmol), P(t-Bu)₃ (1.93 mL, 7.9 mmol), and NaOtBu (11.4 g, 118.51 mmol) were added to a one-neck round bottom flask, and then toluene (100 mL) was added thereto, followed by heating at 135° C. for 10 hours.

When the reaction was completed, extraction was performed with MC and H₂O, followed by column purification to obtain Compound 2-61-1-i (11 g, 84%).

### Preparation of Compound 2-61-2-ii

9H-carbazol-3-ylboronic acid (10 g, 47.3 mmol) and 1000 ml of D₆-benzene were added to a one-neck round bottom flask, and then CF₃SO₃H (170 g, 1075 mmol) was added thereto, followed by stirring at 50° C.

When the reaction was completed, neutralization was performed with D₂O, followed by extraction with MC and an aqueous Na₂CO₃ solution, and then column purification to obtain 2-61-2-ii (9 g, 87%).

### Preparation of Compound 2-61-2-i

2-61-2-ii (9 g, 41.3 mmol), bromobenzene (12.9 g, 82.5 mmol) [E], Pd₂(dba)₃ (1.89 g, 2.06 mmol), P(t-Bu)₃ (2 mL, 8.25 mmol), and NaOtBu (7.93 g, 82.54 mmol) were added to a one-neck round bottom flask, and then toluene (100 mL) was added thereto, followed by heating at 135° C. for 10 hours.

When the reaction was completed, extraction was performed with MC and _{H2O}, followed by column purification to obtain 2-61-2-i (10 g, 82%).

### Preparation of Compound 2-61[F]

2-[F]-61-i (10 g, 30.37 mmol), 2-61-2-ii (17.87 g, 60.75 mmol), Pd(PPh₃)₄ (1.39 g, 1.52 mmol), and K₂CO₃ (12.59 g, 91.13 mmol) were added to a one-neck round bottom flask, and then a mixture of 1,4-dioxane/water (140 ml/35 ml) was heated at 120° C. for 4 hours.

After the reaction was terminated, the temperature was lowered to room temperature, and the generated solid was washed with distilled water and MeOH to obtain the target Compound 2-61[F] (13 g, 85%).

In Preparation Example 3, a target Compound F was prepared in the same manner except that [D] and [E] were respectively replaced with Intermediates D and E of Table 3 below.

TABLE 3

| | Intermediate | Intermediate | Target Compound | Yeild |
|---|---|---|---|---|
| No. | D | E | F | (%) |
| 2-61 | 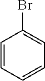 | 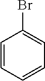 | 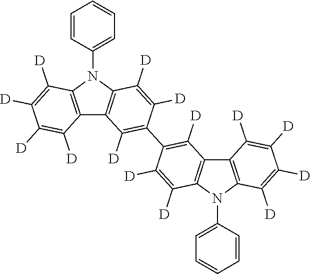 | 85 |
| 2-62 | 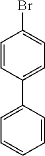 | 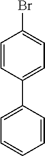 | 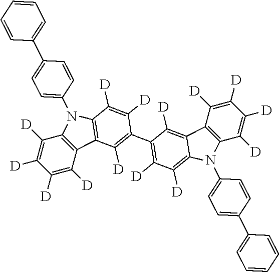 | 75% |
| 2-63 | 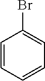 | 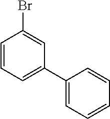 | 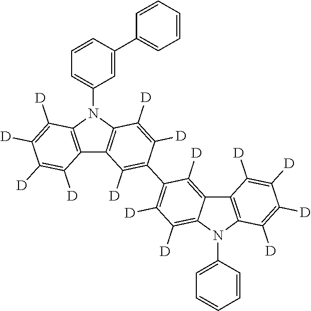 | 73% |
| 2-64 | 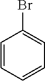 | 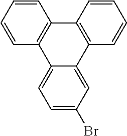 | 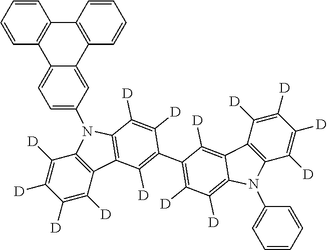 | 66% |
| 2-65 | 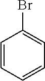 | 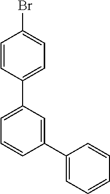 | 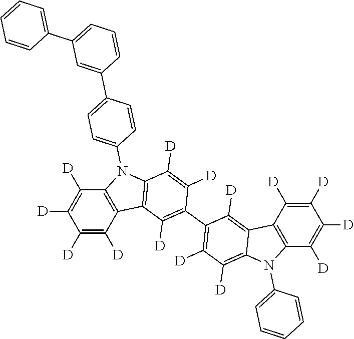 | 69% |
| 2-66 | 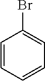 | 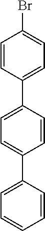 | 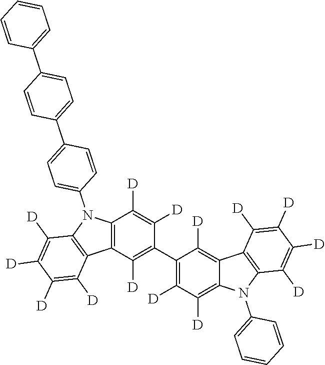 | 75% |
| 2-68 | 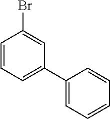 | 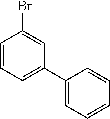 | 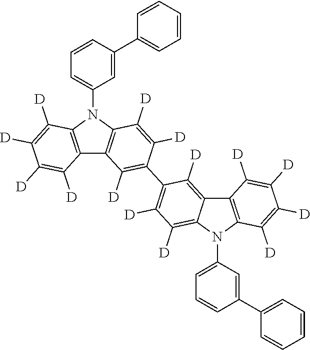 | 78% |
| 2-69 | 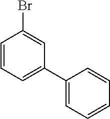 | 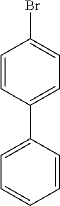 | 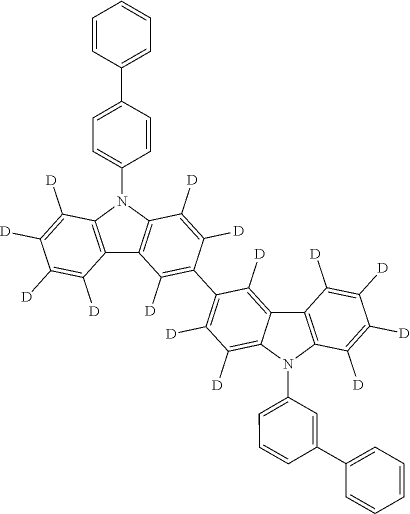 | 82% |
| 2-70 | 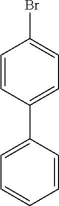 | 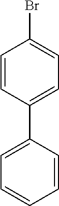 | 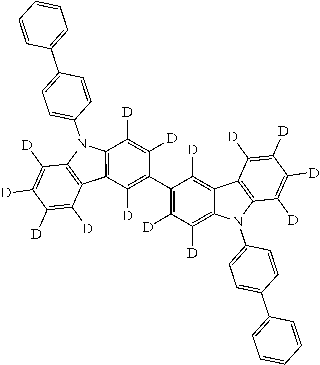 | 67% |
| 2-74 | 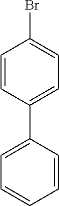 | 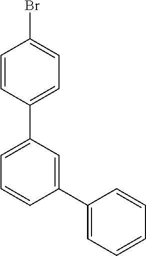 | 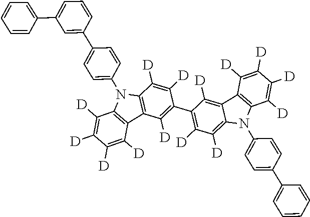 | 68% |
| 2-75 | 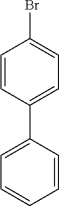 | 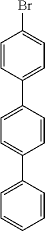 | 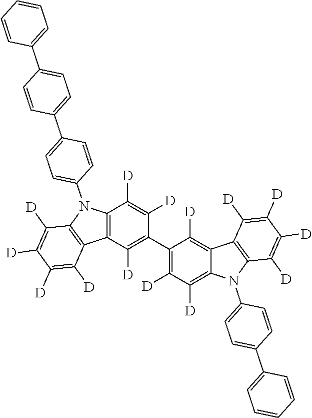 | 75% |
| 2-81 | 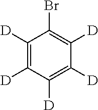 | 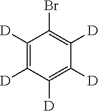 | 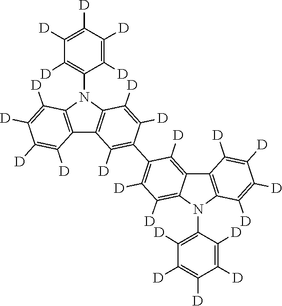 | 64% |
| 2-82 | 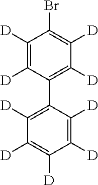 | 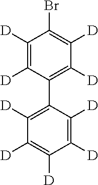 | 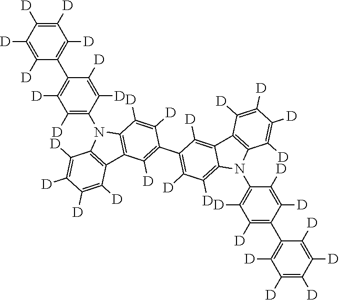 | 78% |
| 2-83 | 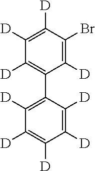 | 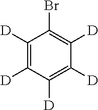 | 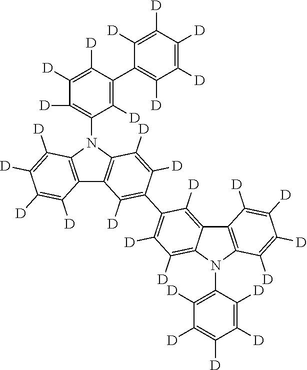 | 70% |
| 2-85 | 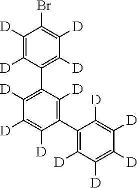 | 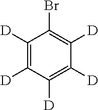 | 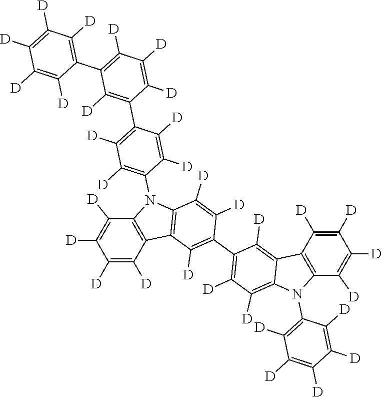 | 70% |
| 2-86 | 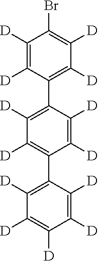 | 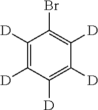 | 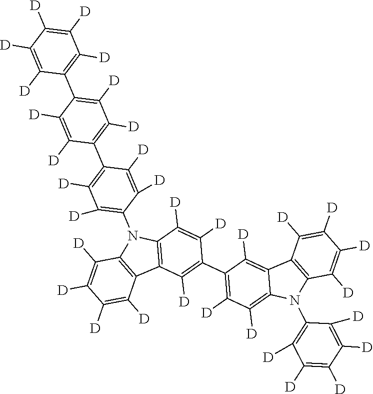 | 66% |
| 2-87 | 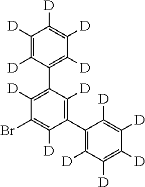 | 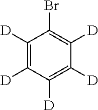 | 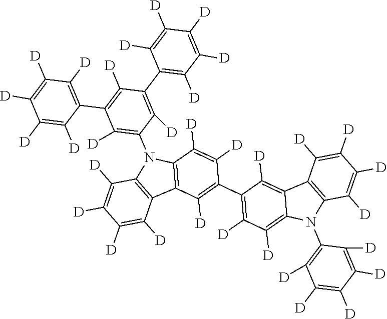 | 77% |
| 2-88 | 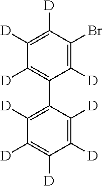 | 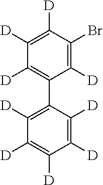 | 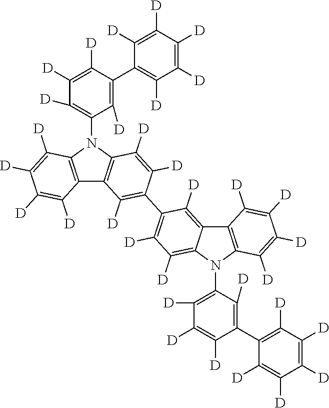 | 75% |
| 2-89 | 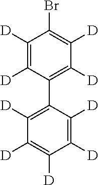 | 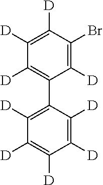 | 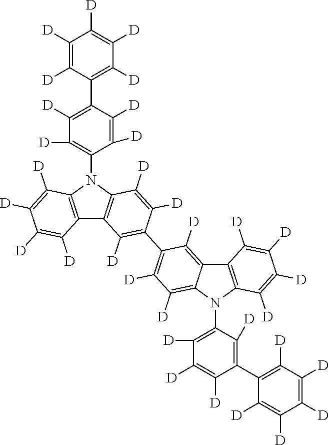 | 67% |
| 2-90 | 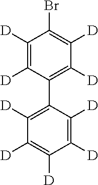 | 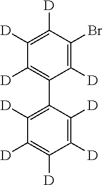 | 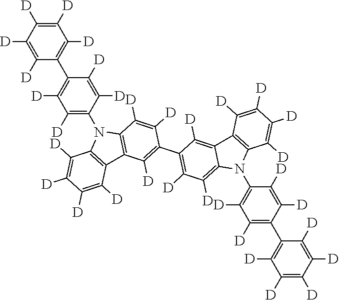 | 73% |
| 2-92 | 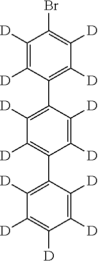 | 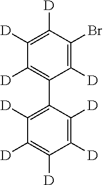 | 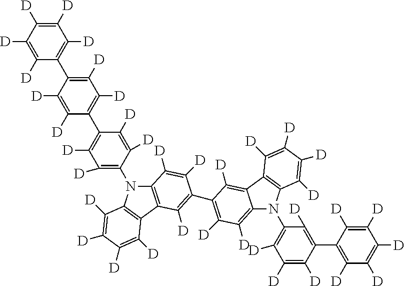 | 68% |
| 2-102 | 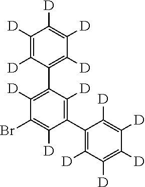 | 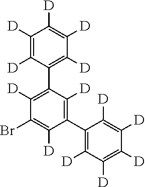 | 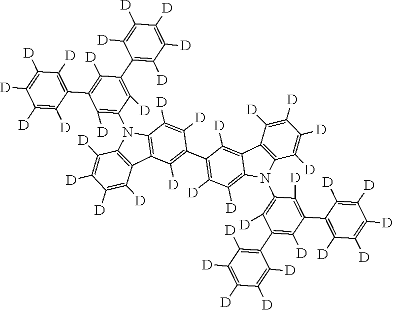 | 70% |

### [Preparation Example 4] Preparation of Compound 3-78 [D]

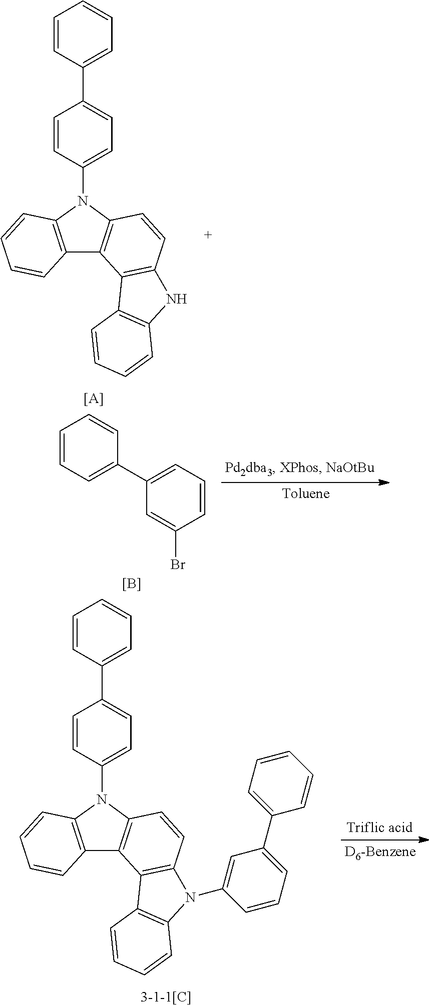

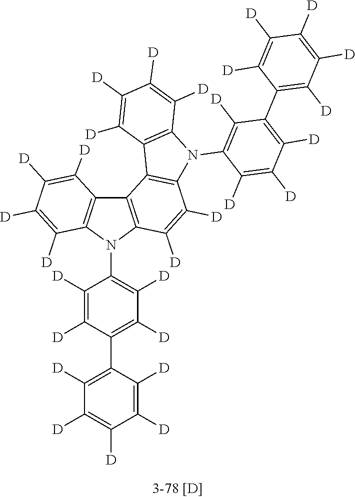 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H]]

### Preparation of Compound 3-1-1[C]

5-([1,1′-biphenyl]-4-yl)-5,8-dihydroindolo[2,3-c]carbazole (10 g, 24.48 mmol) [A], 3-bromobiphenyl(5.82 g, 24.48 mmol) [B], Pd₂(dba)₃ (1.12 g, 1.22 mmol), XPhos (1.17 g, 2.45 mmol), and NaOtBu (7.06 g, 73.44 mmol) were added to a one-neck round bottom flask, and then toluene (100 mL) was added thereto, followed by heating at 135° C. for 15 hours.

When the reaction was completed, extraction was performed with MC and H₂O, followed by column purification to obtain the target Compound 3-1-1[C] (12 g, 87%).

### Preparation of Compound 3-78 [D]

5-([1,1′-biphenyl]-3-yl)-8-([1,l′-biphenyl]-4-yl)-5,8-dihydroindolo[2,3-c]carbazole (12 g, 21.4 mmol) 3-1-1 [C] and 100 mL of D₆-benzene were added to a one-neck round bottom flask, and stirred in an ice bath.

Triflic acid (18.89 mL, 214.02 mmol) was added dropwise thereto, followed by stirring at 50° C. for 1 hour. After the reaction was terminated, the reactor was transferred to an ice bath, and then an aqueous solution prepared in a supersaturated state by dissolving K₃PO₄ (45.43 g, 214.02 mmol) in 300 mL of D₂O was added dropwise to the reactor to neutralize. Extraction was performed with MC and H₂O, followed by column purification to obtain the target Compound 3-78 [D] (10 g, 79%).

In Preparation Example 4, a target Compound C was prepared in the same manner except that [A] and [B] were respectively replaced with Intermediates A and B of Table 4 below.

TABLE 4

| | Intermediate | Intermediate | Target compound | Yeild |
|---|---|---|---|---|
| No. | A | B | C | (%) |
| 2-78 | 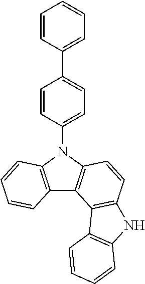 | 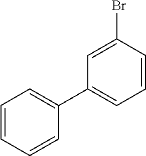 | 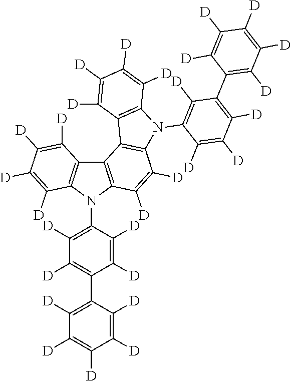 | 79 |
| 2-89 | 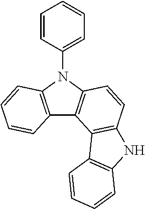 | 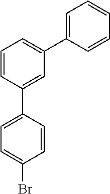 | 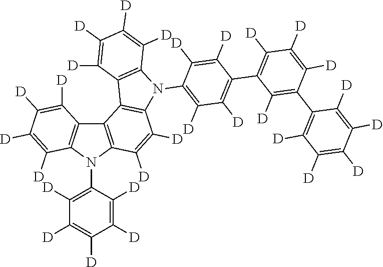 | 76 |
| 2-91 | 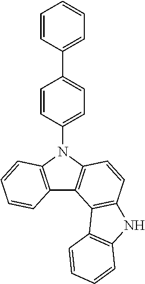 | 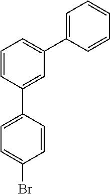 | 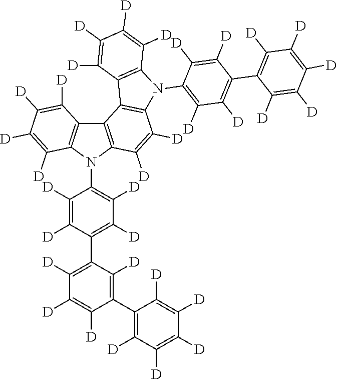 | 81 |
| 2-122 | 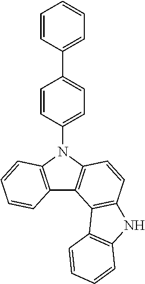 | 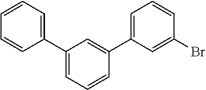 | 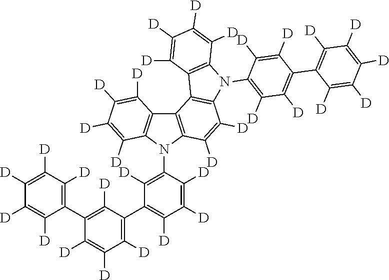 | 78 |
| 2-124 | 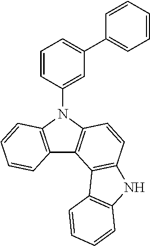 | 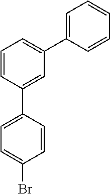 | 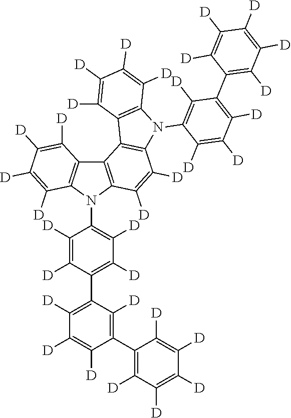 | 80 |

Compounds were prepared in the same manner as the above preparation examples, and the synthesis confirmation results thereof are shown in Tables 5 and 6 below. Table 5 below shows measured values of a field desorption mass spectrometer (FD-MS), and Table 6 below shows NMR values.

TABLE 5

| Compound | FD-Mass |
|---|---|
| 1-1 | m/z = 721.88 (C51H27D6N5 = 721.31) |
| 1-2 | m/z = 723.89 (C51H25D8N5 = 723.32) |
| 1-3 | m/z = 729.93 (C51H19D14N5 = 729.36) |
| 1-4 | m/z = 722.88 (C51H26D7N5 = 722.32) |
| 1-5 | m/z = 723.89 (C51H25D8N5 = 723.32) |
| 1-6 | m/z = 730.93 (C51H18D15N5 = 730.37) |
| 1-7 | m/z = 721.88 (C51H27D6N5 = 721.31) |
| 1-8 | m/z = 723.89 (C51H25D8N5 = 723.32) |
| 1-9 | m/z = 729.93 (C51H19D14N5 = 729.36) |
| 1-10 | m/z = 722.88 (C51H26D7N5 = 722.32) |
| 1-11 | m/z = 723.89 (C51H25D8N5 = 723.32) |
| 1-12 | m/z = 730.93 (C51H18D15N5 = 730.37) |
| 1-25 | m/z = 722.88 (C51H26D7N5 = 722.32) |
| 1-26 | m/z = 723.89 (C51H25D8N5 = 723.32) |
| 1-27 | m/z = 730.93 (C51H18D15N5 = 730.37) |
| 1-28 | m/z = 722.88 (C51H26D7N5 = 722.32) |
| 1-29 | m/z = 723.89 (C51H25D8N5 = 723.32) |
| 1-30 | m/z = 730.93 (C51H18D15N5 = 730.37) |
| 1-49 | m/z = 722.88 C51H26D7N5 = 722.32 |
| 1-50 | m/z = 723.89 (C51H25D8N5 = 723.32) |
| 1-51 | m/z = 730.93 (C51H18D15N5 = 730.37) |
| 1-52 | m/z = 722.88 (C51H26D7N5 = 722.32) |
| 1-53 | m/z = 723.89 (C51H25D8N5 = 723.32) |
| 1-54 | m/z = 730.93 (C51H18D15N5 = 730.37) |
| 1-55 | m/z = 722.88 (C51H26D7N5 = 722.32) |
| 1-55 | m/z = 723.89 (C51H25D8N5 = 723.32) |
| 1-56 | m/z = 730.93 (C51H18D15N5 = 730.37) |
| 1-57 | m/z = 722.88 (C51H26D7N5 = 722.32) |
| 1-58 | m/z = 723.89 (C51H25D8N5 = 723.32) |
| 1-59 | m/z = 730.93 (C51H18D15N5 = 730.37) |
| 1-79 | m/z = 798.98 (C57H30D7N5 = 798.35) |
| 1-80 | m/z = 799.99 (C57H29D8N5 = 799.36) |
| 1-81 | m/z = 807.03 (C57H22D15N5 = 806.4) |
| 1-82 | m/z = 798.98 (C57H30D7N5 = 798.35) |
| 1-83 | m/z = 799.99 (C57H29D8N5 = 799.36) |
| 1-84 | m/z = 807.03 (C57H22D15N5 = 806.4) |
| 1-110 | m/z = 799.99 (C57H29D8N5 = 799.36) |
| 1-111 | m/z = 807.03 (C57H22D15N5 = 806.4) |
| 1-112 | m/z = 798.98 (C57H30D7N5 = 798.35) |
| 1-113 | m/z = 799.99 (C57H29D8N5 = 799.36) |
| 1-114 | m/z = 807.03 (C57H22D15N5 = 806.4) |
| 1-115 | m/z = 798.98 (C57H30D7N5 = 798.35) |
| 1-135 | m/z = 807.03 (C57H22D15N5 = 806.4) |
| 1-138 | m/z = 807.03 (C57H22D15N5 = 806.4) |
| 1-147 | m/z = 807.03 (C57H22D15N5 = 806.4) |
| 1-150 | m/z = 807.03 (C57H22D15N5 = 806.4) |
| 1-213 | m/z = 807.03 (C57H22D15N5 = 806.4) |
| 1-216 | m/z = 807.03 (C57H22D15N5 = 806.4) |
| 1-243 | m/z = 807.03 (C57H22D15N5 = 806.4) |
| 1-339 | m/z = 807.03 (C57H22D15N5 = 806.4) |
| 1-342 | m/z = 807.03 (C57H22D15N5 = 806.4) |
| 1-387 | m/z = 807.03 (C57H22D15N5 = 806.4) |
| 1-414 | m/z = 807.03 (C57H22D15N5 = 806.4) |
| 1-421 | m/z = 798.98 (C57H30D7N5 = 798.35) |
| 1-424 | m/z = 897.11 (C63H24D15N50 = 896.41) |
| 1-425 | m/z = 810.05 (C57H19D18N5 = 809.42) |
| 1-429 | m/z = 813.97 (C57H27D8N50 = 813.33) |
| 1-430 | m/z = 897.11 (C63H24D15N50 = 896.41) |
| 1-437 | m/z = 820.01 (C57H21D14N50 = 819.37) |
| 1-439 | m/z = 836.07 (C57H21D14N5S = 835.35) |
| 1-441 | m/z = 820.03 (C57H21D15N6 = 819.39) |
| 1-448 | m/z = 913.17 (C63H24D15N5S = 912.39) |
| 1-453 | m/z = 913.17 (C63H24D15N5S = 912.39) |
| 1-463 | m/z = 807.03 (C57H22D15N5 = 806.4) |

TABLE 6

| | 1H NMR(DMSO, 300 MHz) |
|---|---|
| 1-1 | δ = 8.55 (1H, d), 8.28 (4H, d), 8.12 (1H, d), 7.98 (1H, s), 7.94 (1H, d), 7.88 (1H, s), 7.75-7.74 (2H, |
| | s), 7.63 (1H, d), 7.51-7.19 (15H, m) |
| 1-2 | δ = 8.55 (1H, d), 8.28 (4H, d), 7.98 (1H, s), 7.94 (1H, d), 7.88 (1H, s), 7.75-7.74 (2H, s), 7.63 (1H, |
| | d), 7.51-7.19 (14H, m) |
| 1-3 | δ = 8.28 (4H, d), 7.98 (1H, s), 7.88 (1H, s), 7.75-7.74 (2H, s), 7.51-7.41 (9H, m), 7.19 (2H, d) |
| 1-4 | δ = 8.55 (1H, d), 8.28 (4H, d), 8.12 (1H, d), 7.94 (1H, d), 7.88 (1H, s), 7.75-7.74 (2H, s), 7.63 (1H, |
| | d), 7.51-7.19 (15H, m) |
| 1-5 | δ = 8.55 (1H, d), 8.28 (4H, d), 8.12 (1H, d), 7.94 (1H, d), 7.88 (1H, s), 7.75-7.74 (2H, s), 7.51-7.19 |
| | (15H, m) |
| 1-6 | δ = 8.28 (4H, d), 7.88 (1H, s), 7.75-7.74 (2H, s), 7.51-7.41 (9H, m), 7.19 (2H, d) |
| 1-7 | δ = 8.55 (1H, d), 8.28 (4H, d), 8.15 (1H, s), 8.12 (1H, d), 7.98 (1H, s), 7.94 (1H, d), 7.85 (1H, d), |
| | 7.79 (2H, d), 7.64-7.63 (2H, d), 7.51-7.25 (13H, m) |
| 1-8 | δ = 8.55 (1H, d), 8.28 (4H, d), 8.15 (1H, s), 7.98 (1H, s), 7.94 (1H, d), 7.85 (1H, d), 7.79 (2H, d), |
| | 7.64-7.63 (2H, d), 7.51-7.25 (12H, m) |
| 1-9 | δ = 8.28 (4H, d), 8.15 (1H, d), 7.98 (1H, s), 7.85 (1H, d), 7.79 (2H, d), 7.64 (1H, d), 7.51-7.41 (9H, |
| | m) |
| 1-10 | δ = 8.55 (1H, d), 8.28 (4H, d), 8.15 (1H, s), 8.12 (1H, d), 7.94 (1H, d), 7.85 (1H, d), 7.79 (2H, d), |
| | 7.64-7.63 (2H, d), 7.51-7.25 (13H, m) |
| 1-11 | δ = 8.55 (1H, d), 8.28 (4H, d), 8.15-8.12 (2H, m), 7.94 (1H, d), 7.85 (1H, d), 7.79 (2H, d), 7.64 |
| | (1H, d), 7.51-7.25 (13H, m) |
| 1-12 | δ = 8.28 (4H, d), 8.15 (1H, s), 7.85 (1H, d), 7.79 (2H, d), 7.64 (1H, d), 7.51-7.41 (9H, m) |
| 1-25 | δ = 8.55 (1H, d), 8.28 (4H, d), 8.12 (1H, d), 8.05 (2H, d), 7.94 (1H, d), 7.88 (1H, s), 7.63 (1H, d), |
| | 7.52-7.25 (15H, m) |
| 1-26 | δ = 8.55 (1H, d), 8.28 (4H, d), 8.05 (2H, s), 7.98 (1H, s), 7.94 (1H, d), 7.88 (1H, s), 7.63 (1H, d), |
| | 7.52-7.25 (14H, m) |
| 1-27 | δ = 8.28 (4H, d), 8.05 (2H, s), 7.88 (1H, s), 7.52-7.41 (11H, m) |
| 1-28 | δ = 8.55 (1H, d), 8.28 (4H, d), 8.12 (1H, d), 8.05 (2H, s), 7.94 (1H, d), 7.88 (1H, s), 7.63 (1H, d), |
| | 7.52-7.25 (15H, m) |
| 1-29 | δ = 8.55 (1H, d), 8.28 (4H, d), 8.12 (1H, d), 8.05 (2H, s), 7.94 (1H, d), 7.88 (1H, s), 7.52-7.25 |
| | (15H, m) |
| 1-30 | δ = 8.28 (4H, d), 8.05 (2H, s), 7.88 (1H, s), 7.52-7.41 (11H, m) |
| 1-49 | δ = 8.55 (1H, d), 8.28 (4H, d), 8.15-8.12 (2H, m), 7.94 (1H, d), 7.85 (1H, d), 7.63 (1H, d), 7.52- |
| | 7.25 (16H, m) |
| 1-50 | δ = 8.55 (1H, d), 8.28 (4H, d), 8.15 (1H, s), 7.98 (1H, s), 7.94 (1H, d), 7.85 (1H, d), 7.63 (1H, d), |
| | 7.52-7.25 (15H, m) |
| 1-51 | δ = 8.28 (4H, d), 8.15 (1H, s), 7.85 (1H, d), 7.52-7.41 (11H, m), 7.25 (1H, d) |
| 1-52 | δ = 8.55 (1H, d), 8.28 (4H, d), 8.15 (1H, s), 8.12 (1H, d), 7.94 (1H, d), 7.85 (1H, d), 7.63 (1H, d), |
| | 7.52-7.25 (16H, m) |
| 1-53 | δ = 8.55 (1H, d), 8.28 (4H, d), 8.15-8.12 (2H, d), 7.94 (1H, d), 7.85 (1H, d), 7.52-7.25 (16H, m) |
| 1-54 | δ = 8.28 (4H, d), 8.15 (1H, s), 7.85 (1H, d), 7.52-7.41 (11H, m), 7.25 (1H, d) |
| 1-55 | δ = 8.55 (1H, d), 8.28 (4H, d), 8.12 (1H, d), 7.94 (1H, d), 7.75 (2H, d), 7.63-7.19 (17H, m) |
| 1-56 | δ = 8.55 (1H, d), 8.28 (4H, d), 7.98 (1H, s), 7.94 (2H, d), 7.75 (2H, d), 7.63-7.19 (16H, m) |
| 1-57 | δ = 8.28 (4H, d), 7.75 (2H, d), 7.57-7.41 (10H, m), 7.19 (2H, d) |
| 1-58 | δ = 8.55 (1H, d), 8.28 (4H, d), 8.12 (1H, d), 7.94 (1H, d), 7.75 (2H, d), 7.63-7.19 (17H, m) |
| 1-59 | δ = 8.55 (1H, d), 8.28 (4H, d), 8.12 (1H, d), 7.94 (1H, d), 7.75 (2H, d), 7.57 (1H, d), 7.51-7.19 |
| | (15H, m) |
| 1-60 | δ = 8.28 (2H, d), 7.75 (2H, d), 7.57-7.41 (10H, m), 7.19 (2H, d) |
| 1-79 | δ = 8.55 (1H, d), 8.28 (4H, d), 8.12 (1H, d), 7.94 (1H, d), 7.88-7.85 (3H, m), 7.75-7.74 (2H, s), |
| | 7.63 (1H, d), 7.51-7.25 (17H, m) |
| 1-80 | δ = 8.55 (1H, d), 8.28 (4H, d), 7.98 (1H, s), 7.94 (1H, d), 7.88-7.85 (3H, m), 7.75-7.74 (2H, s), 7.63 |
| | (1H, d), 7.51-7.19 (16H, m) |
| 1-81 | δ = 8.28 (4H, d), 7.88-7.85 (3H, m), 7.75-7.74 (2H, s), 7.51-7.41 (9H, m), 7.25 (2H, d), 7.19 (2H, |
| | d) |
| 1-82 | δ = 8.55 (1H, d), 8.28 (4H, d), 8.12 (1H, d), 7.94 (1H, d), 7.88-7.85 (3H, m), 7.75-7.74 (2H, s), |
| | 7.63 (1H, d), 7.51-7.19 (17H, m) |
| 1-83 | δ = 8.55 (1H, d), 8.28 (4H, d), 8.12 (1H, d), 7.94 (1H, d), 7.88-7.85 (3H, m), 7.75-7.74 (2H, s), |
| | 7.51-7.19 (17H, m) |
| 1-84 | δ = 8.28 (4H, d), 7.88-7.85 (3H, m), 7.75-7.74 (2H, s), 7.51-7.35 (9H, m), 7.25 (2H, d), 7.19 (2H, |
| | d) |
| 1-110 | δ = 8.55 (1H, d), 8.28 (4H, d), 8.15 (1H, s), 7.98-7.94 (2H, m), 7.85 (3H, d), 7.63 (1H, d), 7.51- |
| | 7.19 (17H, m) |
| 1-111 | δ = 8.28 (4H, d), 8.15 (1H, s), 7.85 (3H, d), 7.51-7.41 (9H, m), 7.25 (3H, d), 7.19 (2H, d) |
| 1-112 | δ = 8.55 (1H, d), 8.28 (4H, d), 8.15-8.12 (2H, m), 7.94 (1H, d), 7.85 (3H, d), 7.63 (1H, d), 7.51- |
| | 7.19 (18H, m) |
| 1-113 | δ = 8.55 (1H, d), 8.28 (4H, d), 8.15-8.12 (2H, m), 7.94 (1H, d), 7.85 (3H, d), 7.51-7.19 (18H, m) |
| 1-114 | δ = 8.28 (4H, d), 8.15 (1H, s), 7.85 (3H, d), 7.51-7.41 (9H, m), 7.25 (3H, d), 7.19 (2H, d) |
| 1-115 | δ = 8.55 (1H, d), 8.28 (4H, d), 8.12 (1H, d), 7.94 (1H, d), 7.85 (3H, d), 7.64-7.63 (2H, m), 7.52- |
| | 7.25 (18H, m) |
| 1-135 | δ = 8.28 (4H, d), 7.85 (3H, d), 7.79 (2H, d), 7.64 (1H, d), 7.51-7.41 (10H, m), 7.25 (2H, d) |
| 1-138 | δ = 8.28 (4H, d), 7.85 (3H, d), 7.79 (2H, d), 7.64 (1H, d), 7.51-7.41 (10, m), 7.25 (2H, d) |
| 1-147 | δ = 8.28 (4H, d), 8.15 (1H, s), 7.85 (3H, d), 7.52-7.41 (11H, m), 7.25 (3H, d) |
| 1-150 | δ = 8.28 (4H, d), 8.15 (1H, s), 7.85 (3H, d), 7.52 (8H, m), 7.41 (3H, m), 7.25 (3H, m) |
| 1-213 | δ = 8.28-8.24 (5H, m), 7.85 (1H, d), 7.7 (1H, s), 7.64-7.41 (15H, m) |
| 1-216 | δ = 8.28-8.24 (5H, m), 7.85 (1H, d), 7.7 (1H, s), 7.64-7.41 (1, 15H) |
| 1-243 | δ = 8.28-8.24 (5H, m), 7.75 (2H, d), 7.7 (1H, s), 7.57-7.41 (12H, m), 7.19 (2H, d) |
| 1-339 | δ = 8.28 (4H, d), 8.09 (1H, s), 7.91 (1H, d), 7.76 (1H, s), 7.52-7.41 (14H, m), 7.21 (1H, d) |
| 1-342 | δ = 8.28 (4H, d), 8.09 (1H, s), 7.91 (1H, d), 7.76 (1H, s), 7.52-7.41 (14H, m), 7.21 (1H, d) |
| 1-387 | δ = 8.28 (4H, d), 7.91 (1H, d), 7.79-7.76 (2H, m), 7.68 (1H, d), 7.54-7.41 (13H, m), 7.21 (1H, d), |
| | (,), (,), (,), |
| 1-414 | δ = 8.28 (4H, d), 8.15 (1H, s), 7.85 (3H, m), 7.52-7.41 (3H, m), 7.25 (1H, d) |
| 1-421 | δ = 8.55 (1H, d), 8.28-8.24 (3H, m), 8.15-8.12 (2H, m), 7.94 (1H, d), 7.85 (1H, d), 7.79 (2H, d), |
| | 7.70 (1H, s), 7.64-7.25 (19H, m) |
| 1-424 | δ = 8.28 (2H, d), 7.95-7.89 (3H, m), 7.79-7.75 (4H, m), 7.68-7.61 (4H, m), 7.52-7.32 (10H, m), |
| | 7.21 (1H, d) |
| 1-425 | δ = 8.28 (4H, d), 7.98 (1H, s), 7.88 (1H, s), 7.75-7.74 (2H, s), 7.51-7.41 (9H, m), 7.19 (2H, d) |
| 1-429 | δ = 8.55 (1H, d), 8.28 (4H, d), 7.98-7.88 (5H, m), 7.75-7.74 (3H, m), 7.66-7.63 (3H, m), 7.51-7.25 |
| | (11H, m) |
| 1-430 | δ = 8.28 (2H, d), 7.95-7.85 (5H, m), 7.75-7.74 (3H, m), 7.66-7.64 (2H, m), 7.51 (4H, m), 7.41-7.19 |
| | (8H, m) |
| 1-437 | δ = 8.28 (4H, d), 7.98 (1H, s), 7.89-7.81 (4H, m), 7.75-7.74 (2H, s), 7.66 (1H, d), 7.51 (4H, m), |
| | 7.41-7.32 (5H, m) |
| 1-439 | δ = 8.45-8.35 (2H, m), 8.28 (4H, d), 8.2 (1H, d), 7.98 (2H, m), 7.88 (1H, s), 7.75-7.74 (2H, s), |
| | 7.58-7.41 (9H, m) |
| 1-441 | δ = 8.55 (1H, d), 8.28 (4H, d), 8.12-8.09 (2H, m), 7.94 (1H, d), 7.79 (1H, d), 7.68-7.63 (2H, m), |
| | 7.51-7.25 (10H, m) |
| 1-448 | δ = 8.45-8.41 (2H, m), 8.28 (4H, d), 8.2 (1H, d), 7.98 (1H, m), 7.88-7.85 (3H, m), 7.75-7.74 (2H, |
| | s), 7.58-7.41 (9H, m), 7.25 (2H, d) |
| 1-453 | δ = 8.45-8.41 (2H, m), 8.28 (4H, d), 8.2 (1H, d), 7.98 (1H, m), 7.88 (1H, m), 7.85 (2H, m), 7.75- |
| | 7.74 (2H, d), 7.58-7.25 (11H, m) |
| 1-463 | δ = 8.28 (4H, d), 8.15 (1H, s), 7.85 (2H, s), 7.52-7.51 (8H, m), 7.41 (3H, m), 7.25 (4H, s) |

### Experimental Example 1

### 1) Fabrication of Organic Light-Emitting Device

A glass substrate on which ITO was thin-film coated to a thickness of 1,500 Å was ultrasonically washed with distilled water. When the washing with distilled water was finished, ultrasonic washing was performed with a solvent such as acetone, methanol, and isopropyl alcohol, and after drying, a UVO treatment was performed for 5 minutes using UV in a UV cleaner. Thereafter, the substrate was transferred to a plasma cleaner (PT), subjected to a plasma treatment in a vacuum state for removing an ITO work function and a residual film, and transferred to a thermal deposition equipment for organic deposition.

A hole injection layer 2-TNATA (4,4′,4″-Tris[2-naphthyl(phenyl)amino]triphenylamine) and a hole transport layer NPB (N,N′-Di(1-naphthyl)-N,N′-diphenyl-(1,1′-biphenyl)-4,4′-diamine), which are common layers, were formed on the ITO transparent electrode (anode).

A light-emitting layer was thermally vacuum-deposited thereon as follows. For the light-emitting layer, a compound described in Table 11 below was used as a host and Ir(ppy)₃ (tris(2-phenylpyridine) iridium) was used as a green phosphorescent dopant, and 400 Å was deposited by doping the host with 7% of Ir(ppy)₃. Subsequently, BCP (bathocuproine) was deposited to a thickness of 60 Å as a hole blocking layer, and Alq₃ was deposited to a thickness of 200 Å thereon as an electron transport layer. Finally, an organic light-emitting device was manufactured by forming an electron injection layer by depositing lithium fluoride (LiF) to a thickness of 10 Å on the electron transport layer, and then forming a cathode by depositing an aluminum (Al) cathode to a thickness of 1200 Å on the electron injection layer.

Meanwhile, all organic compounds necessary for manufacturing the OLED device were vacuum-sublimation-purified under 10⁻⁸ to 10⁻⁶ torr for each material and used for manufacturing the OLED.

### 2) Driving Voltage and Luminous Efficiency of Organic Light-Emitting Device

For the organic light-emitting device manufactured as described above, electroluminescence (EL) characteristics were measured using M7000 manufactured by McScience, and based on the measurement results, T₉₀ was measured when a reference luminance was 6,000 cd/A through a lifespan measurement device (M6000) manufactured by McScience.

Results of measuring a driving voltage, luminous efficiency, a color coordinate (CIE), and a lifespan of the organic light-emitting device manufactured according to the present invention are shown in Table 7 below.

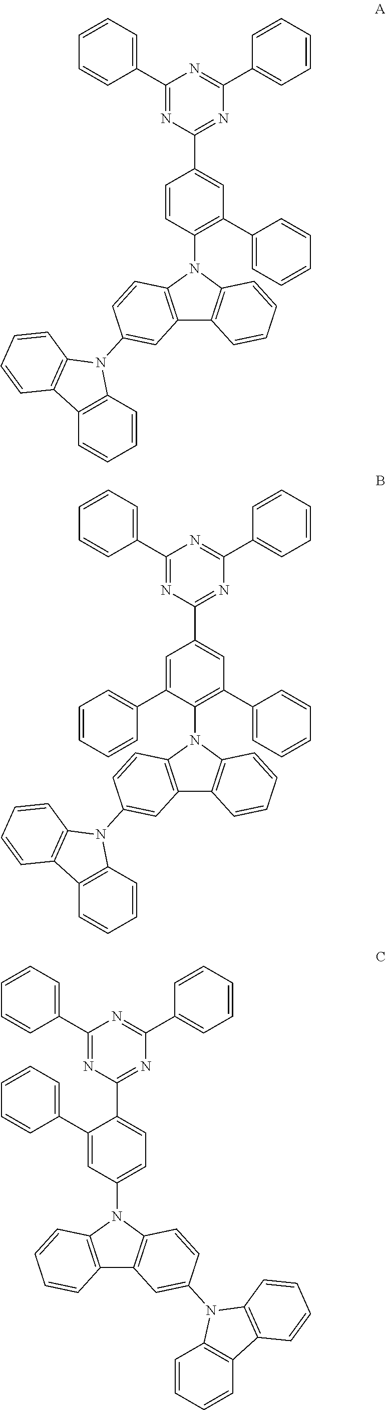

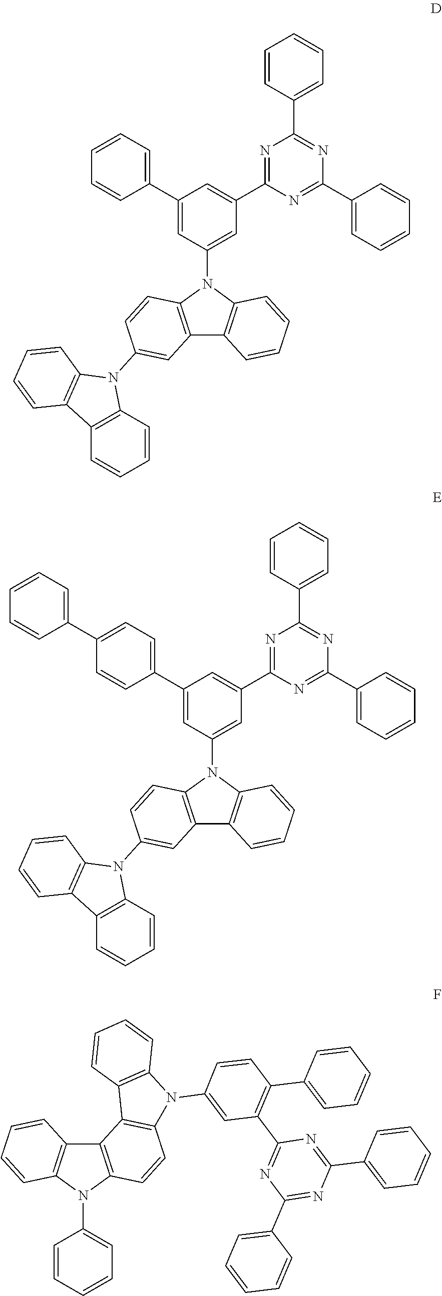 [SMILES: c1ccc(-c2cc(-c3nc(-c4ccccc4)nc(-c4ccccc4)n3)cc(-n3c4ccccc4c4cc(-n5c6ccccc6c6ccccc65)ccc43)c2)cc1 ; c1ccc(-c2ccc(-c3cc(-c4nc(-c5ccccc5)nc(-c5ccccc5)n4)cc(-n4c5ccccc5c5cc(-n6c7ccccc7c7ccccc76)ccc54)c3)cc2)cc1 ; c1ccc(-c2nc(-c3ccccc3)nc(-c3cc(-n4c5ccccc5c5c6c7ccccc7n(-c7ccccc7)c6ccc54)ccc3-c3ccccc3)n2)cc1]

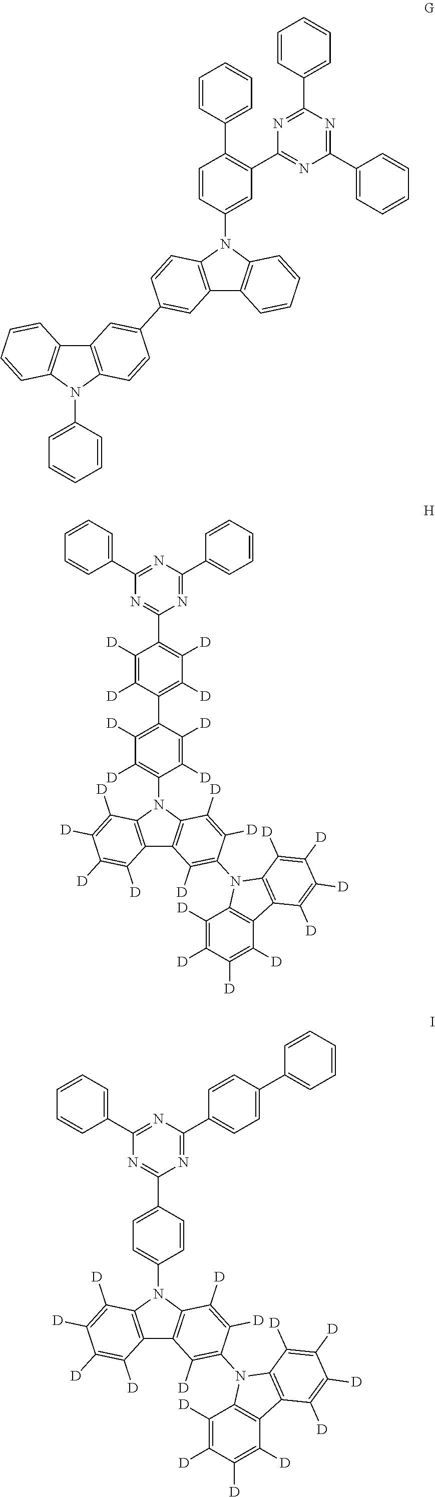

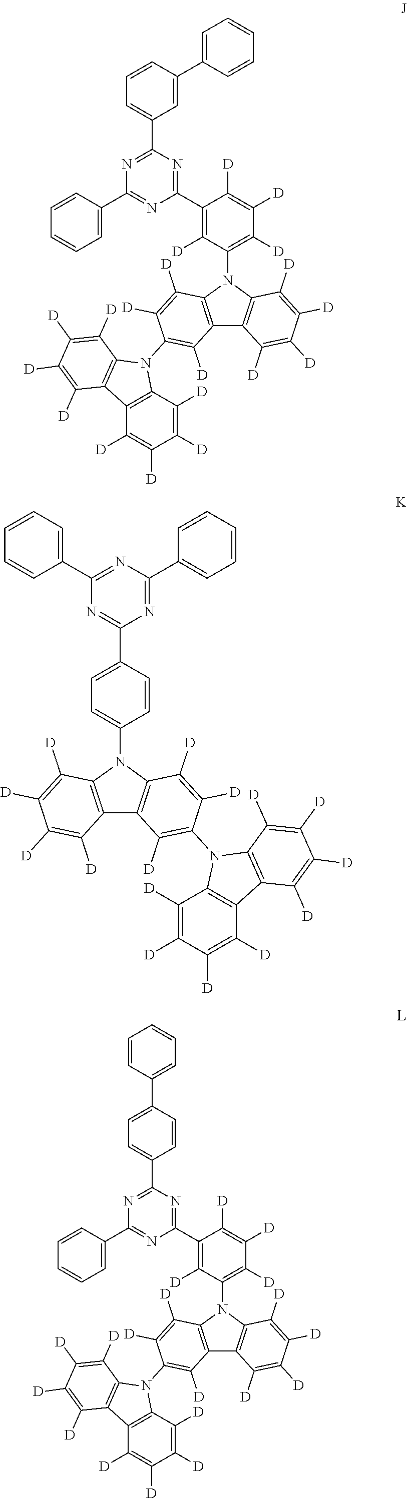

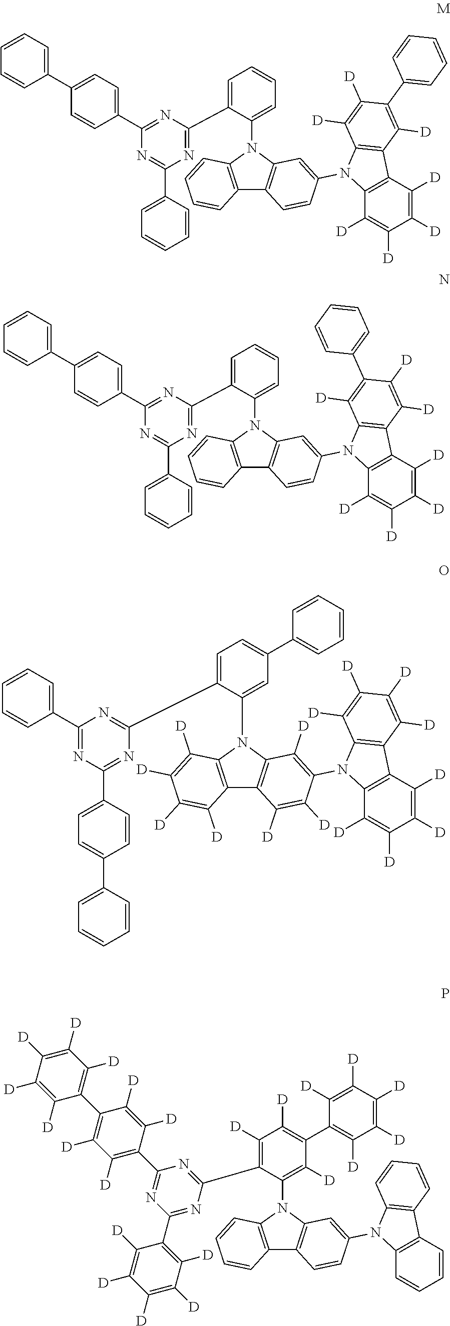

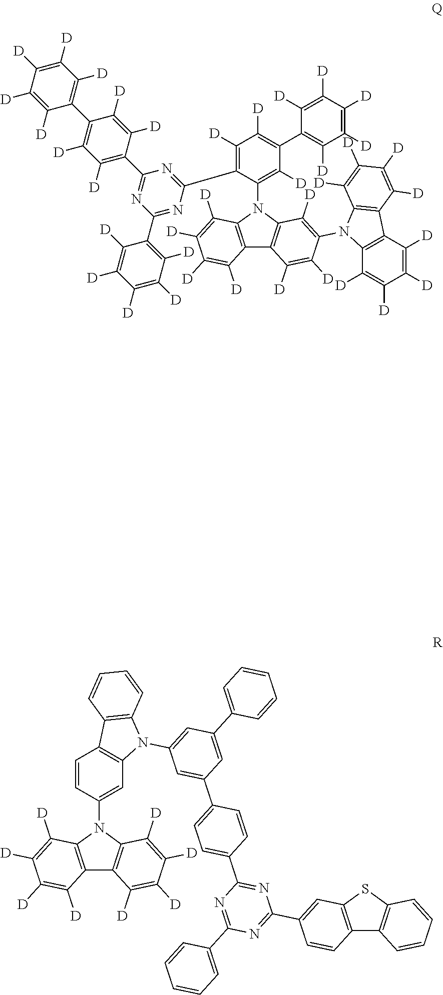 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c(-c4c([2H])c([2H])c([2H])c([2H])c4[2H])c([2H])c3-n3c4c([2H])c([2H])c([2H])c([2H])c4c4c([2H])c([2H])c(-n5c6c([2H])c([2H])c([2H])c([2H])c6c6c([2H])c([2H])c([2H])c([2H])c65)c([2H])c43)n2)c([2H])c1[2H] ; [2H]c1c([2H])c([2H])c2c(c1[2H])c1c([2H])c([2H])c([2H])c([2H])c1n2-c1ccc2c3ccccc3n(-c3cc(-c4ccccc4)cc(-c4ccc(-c5nc(-c6ccccc6)nc(-c6ccc7c(c6)sc6ccccc67)n5)cc4)c3)c2c1]

TABLE 7

| | [symbol] | [symbol] | [symbol] | [symbol] | [symbol] |
|---|---|---|---|---|---|
| | [symbol] | (V) | (cd/A) | (x, y) | (T₉₀) |
| Comparative | A | 3.71 | 101 | (0.272, 0.671) | 81 |
| Example 1 | | | | | |
| Comparative | B | 3.91 | 99 | (0.264, 0.651) | 76 |
| Example 2 | | | | | |
| Comparative | C | 3.72 | 102 | (0.282, 0.652) | 73 |
| Example 3 | | | | | |
| Comparative | D | 3.73 | 100 | (0.265, 0.651) | 68 |
| Example 4 | | | | | |
| Comparative | E | 3.94 | 109 | (0.281, 0.654) | 69 |
| Example 5 | | | | | |
| Comparative | F | 4.01 | 85 | (0.264, 0.652) | 67 |
| Example 6 | | | | | |
| Comparative | G | 3.92 | 81 | (0.281, 0.654) | 76 |
| Example 7 | | | | | |
| Comparative | H | 3.72 | 66 | (0.262, 0.661) | 106 |
| Example 8 | | | | | |
| Comparative | I | 3.75 | 63 | (0.281, 0.653) | 103 |
| Example 9 | | | | | |
| Comparative | J | 3.74 | 67 | (0.269, 0.671) | 101 |
| Example 10 | | | | | |
| Comparative | K | 3.77 | 77 | (0.261, 0.682) | 108 |
| Example 11 | | | | | |
| Comparative | L | 3.71 | 75 | (0.281, 0.654) | 103 |
| Example 12 | | | | | |
| Comparative | M | 3.81 | 75 | (0.264, 0.652) | 83 |
| Example 13 | | | | | |
| Comparative | N | 3.81 | 78 | (0.271, 0.671) | 86 |
| Example 14 | | | | | |
| Comparative | O | 3.79 | 73 | (0.282, 0.652) | 71 |
| Example 15 | | | | | |
| Comparative | P | 3.81 | 79 | (0.271, 0.651) | 65 |
| Example 16 | | | | | |
| Comparative | Q | 3.76 | 81 | (0.264, 0.683) | 91 |
| Example 17 | | | | | |
| Comparative | R | 4.21 | 85 | (0.281, 0.654) | 65 |
| Example 18 | | | | | |
| Example 1 | 1-1 | 3.71 | 124 | (0.271, 0.653) | 205 |
| Example 2 | 1-2 | 3.72 | 125 | (0.262, 0.661) | 207 |
| Example 3 | 1-3 | 3.72 | 126 | (0.261, 0.672) | 268 |
| Example 4 | 1-4 | 3.51 | 138 | (0.272, 0.671) | 186 |
| Example 5 | 1-5 | 3.52 | 139 | (0.276, 0.673) | 187 |
| Example 6 | 1-6 | 3.51 | 140 | (0.271, 0.653) | 242 |
| Example 7 | 1-7 | 3.71 | 122 | (0.281, 0.669) | 186 |
| Example 8 | 1-8 | 3.68 | 124 | (0.273, 0.671) | 187 |
| Example 9 | 1-9 | 3.69 | 125 | (0.276, 0.673) | 242 |
| Example 10 | 1-10 | 3.53 | 139 | (0.271, 0.653) | 161 |
| Example 11 | 1-11 | 3.55 | 140 | (0.266, 0.659) | 163 |
| Example 12 | 1-12 | 3.51 | 141 | (0.271, 0.653) | 210 |
| Example 13 | 1-25 | 3.69 | 114 | (0.276, 0.656) | 181 |
| Example 14 | 1-26 | 3.70 | 117 | (0.266, 0.659) | 188 |
| Example 15 | 1-27 | 3.71 | 114 | (0.262, 0.651) | 257 |
| Example 16 | 1-28 | 3.51 | 141 | (0.264, 0.652) | 145 |
| Example 17 | 1-29 | 3.52 | 142 | (0.264, 0.652) | 148 |
| Example 18 | 1-30 | 3.52 | 142 | (0.271, 0.671) | 201 |
| Example 19 | 1-31 | 3.64 | 113 | (0.282, 0.652) | 189 |
| Example 20 | 1-32 | 3.66 | 116 | (0.271, 0.651) | 191 |
| Example 21 | 1-33 | 3.66 | 117 | (0.272, 0.663) | 252 |
| Example 22 | 1-34 | 3.50 | 164 | (0.268, 0.663) | 164 |
| Example 23 | 1-35 | 3.52 | 166 | (0.264, 0.651) | 166 |
| Example 24 | 1-36 | 3.51 | 167 | (0.267, 0.662) | 215 |
| Example 25 | 1-50 | 3.68 | 118 | (0.262, 0.661) | 191 |
| Example 26 | 1-51 | 3.70 | 120 | (0.271, 0.671) | 268 |
| Example 27 | 1-52 | 3.51 | 171 | (0.276, 0.656) | 156 |
| Example 28 | 1-53 | 3.52 | 170 | (0.266, 0.659) | 155 |
| Example 29 | 1-54 | 3.52 | 172 | (0.262, 0.651) | 207 |
| Example 30 | 1-55 | 3.70 | 120 | (0.264, 0.652) | 191 |
| Example 31 | 1-56 | 3.71 | 121 | (0.265, 0.683) | 193 |
| Example 32 | 1-57 | 3.72 | 119 | (0.269, 0.671) | 271 |
| Example 33 | 1-58 | 3.51 | 146 | (0.261, 0.682) | 155 |
| Example 34 | 1-59 | 3.50 | 147 | (0.281, 0.654) | 152 |
| Example 35 | 1-60 | 3.53 | 143 | (0.281, 0.654) | 210 |
| Example 36 | 1-79 | 3.72 | 141 | (0.281, 0.654) | 171 |
| Example 37 | 1-80 | 3.71 | 140 | (0.281, 0.654) | 172 |
| Example 38 | 1-81 | 3.70 | 142 | (0.268, 0.653) | 241 |
| Example 39 | 1-82 | 3.61 | 171 | (0.267, 0.661) | 151 |
| Example 40 | 1-83 | 3.62 | 172 | (0.265, 0.683) | 155 |
| Example 41 | 1-84 | 3.63 | 170 | (0.265, 0.651) | 201 |
| Example 42 | 1-110 | 3.71 | 144 | (0.274, 0.672) | 181 |
| Example 43 | 1-111 | 3.72 | 145 | (0.264, 0.683) | 180 |
| Example 44 | 1-112 | 3.72 | 145 | (0.281, 0.654) | 269 |
| Example 45 | 1-113 | 3.61 | 170 | (0.275, 0.648) | 160 |
| Example 46 | 1-114 | 3.60 | 169 | (0.282, 0.652) | 159 |
| Example 47 | 1-115 | 3.62 | 171 | (0.281, 0.654) | 209 |
| Example 48 | 1-135 | 3.79 | 142 | (0.268, 0.653) | 251 |
| Example 49 | 1-138 | 3.61 | 169 | (0.265, 0.651) | 200 |
| Example 50 | 1-147 | 3.80 | 139 | (0.268, 0.653) | 252 |
| Example 51 | 1-150 | 3.62 | 173 | (0.265, 0.651) | 203 |
| Example 52 | 1-213 | 3.71 | 140 | (0.268, 0.653) | 255 |
| Example 53 | 1-216 | 3.60 | 176 | (0.265, 0.651) | 200 |
| Example 54 | 1-243 | 3.73 | 147 | (0.268, 0.653) | 249 |
| Example 55 | 1-339 | 3.81 | 142 | (0.268, 0.653) | 241 |
| Example 56 | 1-342 | 3.61 | 181 | (0.265, 0.651) | 199 |
| Example 57 | 1-387 | 3.70 | 139 | (0.268, 0.653) | 242 |
| Example 58 | 1-414 | 3.61 | 184 | (0.265, 0.651) | 198 |
| Example 59 | 1-421 | 3.78 | 119 | (0.281, 0.654) | 181 |
| Example 60 | 1-424 | 3.81 | 139 | (0.281, 0.654) | 201 |
| Example 61 | 1-425 | 3.99 | 151 | (0.275, 0.648) | 231 |
| Example 62 | 1-429 | 4.00 | 131 | (0.269, 0.671) | 205 |
| Example 63 | 1-430 | 3.98 | 123 | (0.265, 0.683) | 265 |
| Example 64 | 1-437 | 3.96 | 137 | (0.269, 0.671) | 309 |
| Example 65 | 1-439 | 3.99 | 139 | (0.261, 0.682) | 321 |
| Example 66 | 1-441 | 3.80 | 168 | (0.281, 0.654) | 247 |
| Example 67 | 1-448 | 3.95 | 141 | (0.281, 0.654) | 316 |
| Example 68 | 1-453 | 3.99 | 146 | (0.261, 0.672) | 320 |
| Example 69 | 1-463 | 3.71 | 141 | (0.261, 0.672) | 311 |

As described in Table 7, the heterocyclic compounds used in Examples 1 to 69 of the present application are characterized by comprising a chemical species in which N-carbazole is bonded to the 1- or 3-position of N-carbazole, and at least six or more deuteriums are substituted in two carbazoles. In addition, it is characterized in that at least one aryl group or heteroaryl group is bonded to a linker (phenyl-L1). Since two N-carbazole groups are bonded at a position (ortho, para) where resonance of unshared electron pairs is not broken, hole transport capability is enhanced, and bonding of at least one aryl group or heteroaryl group to phenyl-L1 introduced as the linker makes a molecular structure more robust, thereby minimizing energy canceled by rotation and vibration, so that it can be confirmed that lifespan and efficiency characteristics of the device are improved. In addition, it was confirmed that the lifespan characteristics of the device were significantly improved by substituting N-carbazole with deuterium.

Comparative Examples 1 to 7 have a structure not comprising deuterium, Comparative Examples 8 to 14 do not comprise a linker having a form in which at least one aryl group or heteroaryl group is bonded to phenyl-L1, and in Comparative Examples 15 to 18, N-carbazole and N-carbazole are bonded at a position 2 (meta), so that expansion of resonance of a HOMO region may be limited. Accordingly, in the case of the organic light-emitting devices of Examples 1 to 69 comprising the heterocyclic compound of the present invention, it can be confirmed that all of the driving voltage, the luminous efficiency, and the lifespan are superior to those of Comparative Examples 1 to 18. This may be determined to be an effect caused by expansion of HOMO, maintenance of charge balance, and improvement in stability of the molecular structure due to a bonding position between N-carbazoles, a phenyl-L1 linker, and introduction of deuterium.

### Experimental Example 2

### 1) Fabrication of Organic Light-Emitting Device

A glass substrate on which ITO was thin-film coated to a thickness of 1,500 Å was ultrasonically washed with distilled water. When the washing with distilled water was finished, ultrasonic washing was performed with a solvent such as acetone, methanol, and isopropyl alcohol, and after drying, a UVO treatment was performed for 5 minutes using UV in a UV cleaner. Thereafter, the substrate was transferred to a plasma cleaner (PT), subjected to a plasma treatment in a vacuum state for removing an ITO work function and a residual film, and transferred to a thermal deposition equipment for organic deposition.

A hole injection layer 2-TNATA (4,4′,4″-Tris[2-naphthyl(phenyl)amino]triphenylamine) and a hole transport layer NPB (N,N′-Di(1-naphthyl)-N,N′-diphenyl-(1,1′-biphenyl)-4,4′-diamine), which are common layers, were formed on the ITO transparent electrode (anode).

A light-emitting layer was thermally vacuum-deposited thereon as follows. The light-emitting layer comprised one heterocyclic compound having the structure of Chemical Formula 1 and one compound having the structure of Chemical Formula 3 or 4 as a host in a ratio (weight ratio) described in Table 11 below, Ir(ppy)₃ (tris(2-phenylpyridine) iridium) was used as a green phosphorescent dopant, and 400 Å was deposited by doping the host with 7% of Ir(ppy)₃. Subsequently, BCP (bathocuproine) was deposited to a thickness of 60 Å as a hole blocking layer, and _{Alq3} was deposited to a thickness of 200 Å thereon as an electron transport layer. Finally, an organic light-emitting device was manufactured by forming an electron injection layer by depositing lithium fluoride (LiF) to a thickness of 10 Å on the electron transport layer, and then forming a cathode by depositing an aluminum (Al) cathode to a thickness of 1200 Å on the electron injection layer.

Meanwhile, all organic compounds necessary for manufacturing the OLED device were vacuum-sublimation-purified under 10⁻⁸ to 10⁻⁶ torr for each material and used for manufacturing the OLED.

### 2) Driving Voltage and Luminous Efficiency of Organic Light-Emitting Device

For the organic light-emitting device manufactured as described above, electroluminescence (EL) characteristics were measured using M7000 manufactured by McScience, and based on the measurement results, T₉₀ was measured when a reference luminance was 6,000 cd/A through a lifespan measurement device (M6000) manufactured by McScience.

Results of measuring a driving voltage, luminous efficiency, a color coordinate (CIE), and a lifespan of the organic light-emitting device manufactured according to the present invention are shown in Table 8 below.

TABLE 8

| | Light- | Light- | | | | | |
|---|---|---|---|---|---|---|---|
| | Emitting | Emitting | | Driving | | color | |
| | layer | layer | | Voltage | Efficiency | coordinates | lifetime |
| | compound | compound | Ratio | (V) | (cd/A) | (x, y) | (T₉₀) |
| Comparative | A | 2-88 | 1:1 | 3.90 | 152 | (0.272, 0.674) | 97 |
| Example 19 | A | 2-88 | 1:2 | 4.08 | 141 | (0.265, 0.683) | 101 |
| | A | 2-88 | 1:3 | 4.16 | 131 | (0.269, 0.671) | 89 |
| Comparative | I | 2-89 | 1:1 | 3.94 | 95 | (0.261, 0.682) | 124 |
| Example 20 | I | 2-89 | 1:2 | 4.13 | 88 | (0.261, 0.672) | 129 |
| | I | 2-89 | 1:3 | 4.20 | 82 | (0.272, 0.671) | 113 |
| Comparative | K | 3-121 | 1:1 | 4.15 | 100 | (0.276, 0.673) | 130 |
| Example 21 | K | 3-121 | 1:2 | 4.52 | 92 | (0.279, 0.651) | 140 |
| | K | 3-121 | 1:3 | 4.71 | 85 | (0.268, 0.653) | 119 |
| Comparative | O | 3-122 | 1:1 | 4.17 | 95 | (0.267, 0.661) | 85 |
| Example 22 | O | 3-122 | 1:2 | 4.55 | 88 | (0.265, 0.683) | 92 |
| | O | 3-122 | 1:3 | 4.74 | 80 | (0.265, 0.651) | 78 |
| Example 70 | 1-3 | 2-82 | 1:1 | 3.91 | 189 | (0.274, 0.672) | 402 |
| | 1-3 | 2-82 | 1:2 | 4.09 | 176 | (0.264, 0.683) | 482 |
| | 1-3 | 2-82 | 1:3 | 4.17 | 164 | (0.281, 0.654) | 348 |
| Example 71 | 1-6 | 2-83 | 1:1 | 3.69 | 210 | (0.275, 0.648) | 363 |
| | 1-6 | 2-83 | 1:2 | 3.86 | 196 | (0.282, 0.652) | 436 |
| | 1-6 | 2-83 | 1:3 | 3.93 | 182 | (0.271, 0.651) | 315 |
| Example 72 | 1-9 | 2-84 | 1:1 | 3.87 | 188 | (0.272, 0.674) | 363 |
| | 1-9 | 2-84 | 1:2 | 4.06 | 175 | (0.265, 0.683) | 436 |
| | 1-9 | 2-84 | 1:3 | 4.13 | 163 | (0.269, 0.671) | 315 |
| Example 73 | 1-12 | 2-88 | 1:1 | 3.69 | 212 | (0.261, 0.682) | 315 |
| | 1-12 | 2-88 | 1:2 | 3.86 | 197 | (0.261, 0.672) | 378 |
| | 1-12 | 2-88 | 1:3 | 3.93 | 183 | (0.272, 0.671) | 273 |
| Example 74 | 1-27 | 2-106 | 1:1 | 3.90 | 171 | (0.276, 0.673) | 386 |
| | 1-27 | 2-106 | 1:2 | 4.08 | 160 | (0.279, 0.651) | 463 |
| | 1-27 | 2-106 | 1:3 | 4.16 | 148 | (0.268, 0.653) | 334 |
| Example 75 | 1-30 | 2-82 | 1:1 | 3.70 | 213 | (0.267, 0.661) | 302 |
| | 1-30 | 2-82 | 1:2 | 3.87 | 199 | (0.265, 0.683) | 362 |
| | 1-30 | 2-82 | 1:3 | 3.94 | 185 | (0.265, 0.651) | 261 |
| Example 76 | 1-33 | 2-83 | 1:1 | 3.84 | 176 | (0.274, 0.672) | 378 |
| | 1-33 | 2-83 | 1:2 | 4.03 | 164 | (0.264, 0.683) | 454 |
| | 1-33 | 2-83 | 1:3 | 4.10 | 152 | (0.281, 0.654) | 328 |
| Example 77 | 1-36 | 2-84 | 1:1 | 3.69 | 251 | (0.275, 0.648) | 323 |
| | 1-36 | 2-83 | 1:2 | 3.86 | 234 | (0.282, 0.652) | 387 |
| | 1-36 | 2-83 | 1:3 | 3.93 | 217 | (0.271, 0.651) | 280 |
| Example 78 | 1-52 | 2-88 | 1:1 | 3.69 | 257 | (0.272, 0.663) | 234 |
| | 1-52 | 2-88 | 1:2 | 3.86 | 239 | (0.272, 0.674) | 281 |
| | 1-52 | 2-88 | 1:3 | 3.93 | 222 | (0.265, 0.683) | 203 |
| Example 79 | 1-55 | 2-106 | 1:1 | 3.89 | 180 | (0.269, 0.671) | 287 |
| | 1-55 | 2-106 | 1:2 | 4.07 | 168 | (0.261, 0.682) | 344 |
| | 1-55 | 2-106 | 1:3 | 4.14 | 156 | (0.261, 0.672) | 248 |
| Example 80 | 1-58 | 2-82 | 1:1 | 3.69 | 219 | (0.272, 0.671) | 233 |
| | 1-58 | 2-82 | 1:2 | 3.86 | 204 | (0.276, 0.673) | 279 |
| | 1-58 | 2-82 | 1:3 | 3.93 | 190 | (0.279, 0.651) | 202 |
| Example 81 | 1-60 | 2-83 | 1:1 | 3.71 | 215 | (0.268, 0.653) | 315 |
| | 1-60 | 2-83 | 1:2 | 3.88 | 200 | (0.267, 0.661) | 378 |
| | 1-60 | 2-83 | 1:3 | 3.95 | 186 | (0.265, 0.683) | 273 |
| Example 82 | 1-81 | 2-84 | 1:1 | 3.89 | 213 | (0.274, 0.672) | 362 |
| | 1-81 | 2-84 | 1:2 | 4.07 | 199 | (0.264, 0.683) | 434 |
| | 1-81 | 2-84 | 1:3 | 4.14 | 185 | (0.281, 0.654) | 313 |
| Example 83 | 1-425 | 2-88 | 1:1 | 4.19 | 227 | (0.275, 0.648) | 347 |
| | 1-425 | 2-88 | 1:2 | 4.39 | 211 | (0.282, 0.652) | 416 |
| | 1-425 | 2-88 | 1:3 | 4.47 | 196 | (0.271, 0.651) | 300 |
| Example 84 | 1-429 | 2-106 | 1:1 | 4.20 | 197 | (0.272, 0.674) | 308 |
| | 1-429 | 2-106 | 1:2 | 4.40 | 183 | (0.265, 0.683) | 369 |
| | 1-429 | 2-106 | 1:3 | 4.48 | 170 | (0.269, 0.671) | 267 |
| Example 85 | 1-3 | 3-78 | 1:1 | 4.09 | 164 | (0.261, 0.682) | 456 |
| | 1-3 | 3-78 | 1:2 | 4.46 | 151 | (0.261, 0.672) | 509 |
| | 1-3 | 3-78 | 1:3 | 4.65 | 139 | (0.272, 0.671) | 402 |
| Example 86 | 1-6 | 3-89 | 1:1 | 3.86 | 182 | (0.276, 0.673) | 411 |
| | 1-6 | 3-89 | 1:2 | 4.21 | 168 | (0.279, 0.651) | 460 |
| | 1-6 | 3-89 | 1:3 | 4.39 | 154 | (0.268, 0.653) | 363 |
| Example 87 | 1-9 | 3-91 | 1:1 | 4.06 | 163 | (0.267, 0.661) | 411 |
| | 1-9 | 3-91 | 1:2 | 4.43 | 150 | (0.265, 0.683) | 460 |
| | 1-9 | 3-91 | 1:3 | 4.61 | 138 | (0.265, 0.651) | 363 |
| Example 88 | 1-12 | 3-122 | 1:1 | 3.86 | 183 | (0.274, 0.672) | 357 |
| | 1-12 | 3-122 | 1:2 | 4.21 | 169 | (0.264, 0.683) | 399 |
| | 1-12 | 3-122 | 1:3 | 4.39 | 155 | (0.281, 0.654) | 315 |
| Example 89 | 1-27 | 3-124 | 1:1 | 4.08 | 148 | (0.275, 0.648) | 437 |
| | 1-27 | 3-124 | 1:2 | 4.45 | 137 | (0.282, 0.652) | 488 |
| | 1-27 | 3-124 | 1:3 | 4.64 | 125 | (0.271, 0.651) | 386 |
| Example 90 | 1-30 | 3-78 | 1:1 | 3.87 | 185 | (0.272, 0.663) | 342 |
| | 1-30 | 3-78 | 1:2 | 4.22 | 170 | (0.272, 0.674) | 382 |
| | 1-30 | 3-78 | 1:3 | 4.40 | 156 | (0.265, 0.683) | 302 |
| Example 91 | 1-33 | 3-89 | 1:1 | 4.03 | 152 | (0.269, 0.671) | 428 |
| | 1-33 | 3-89 | 1:2 | 4.39 | 140 | (0.261, 0.682) | 479 |
| | 1-33 | 3-89 | 1:3 | 4.58 | 129 | (0.261, 0.672) | 378 |
| Example 92 | 1-36 | 3-91 | 1:1 | 3.86 | 217 | (0.272, 0.671) | 366 |
| | 1-36 | 3-91 | 1:2 | 4.21 | 200 | (0.276, 0.673) | 409 |
| | 1-36 | 3-91 | 1:3 | 4.39 | 184 | (0.279, 0.651) | 323 |
| Example 93 | 1-52 | 3-122 | 1:1 | 3.86 | 222 | (0.268, 0.653) | 265 |
| | 1-52 | 3-122 | 1:2 | 4.21 | 205 | (0.267, 0.661) | 296 |
| | 1-52 | 3-122 | 1:3 | 4.39 | 188 | (0.265, 0.683) | 234 |
| Example 94 | 1-55 | 3-124 | 1:1 | 4.07 | 156 | (0.261, 0.672) | 325 |
| | 1-55 | 3-124 | 1:2 | 4.44 | 144 | (0.272, 0.671) | 363 |
| | 1-55 | 3-124 | 1:3 | 4.63 | 132 | (0.276, 0.673) | 287 |
| Example 95 | 1-58 | 3-78 | 1:1 | 3.86 | 190 | (0.279, 0.651) | 264 |
| | 1-58 | 3-78 | 1:2 | 4.21 | 175 | (0.268, 0.653) | 295 |
| | 1-58 | 3-78 | 1:3 | 4.39 | 161 | (0.267, 0.661) | 233 |
| Example 96 | 1-60 | 3-89 | 1:1 | 3.88 | 186 | (0.265, 0.683) | 357 |
| | 1-60 | 3-89 | 1:2 | 4.24 | 172 | (0.274, 0.672) | 399 |
| | 1-60 | 3-89 | 1:3 | 4.41 | 157 | (0.264, 0.683) | 315 |
| Example 97 | 1-81 | 3-91 | 1:1 | 4.07 | 185 | (0.281, 0.654) | 410 |
| | 1-81 | 3-91 | 1:2 | 4.44 | 170 | (0.275, 0.648) | 458 |
| | 1-81 | 3-91 | 1:3 | 4.63 | 156 | (0.282, 0.652) | 362 |
| Example 98 | 1-425 | 3-122 | 1:1 | 4.39 | 196 | (0.271, 0.651) | 393 |
| | 1-425 | 3-122 | 1:2 | 4.79 | 181 | (0.272, 0.674) | 439 |
| | 1-425 | 3-122 | 1:3 | 4.99 | 166 | (0.265, 0.683) | 347 |
| Example 99 | 1-429 | 3-124 | 1:1 | 4.40 | 170 | (0.269, 0.671) | 349 |
| | 1-429 | 3-124 | 1:2 | 4.80 | 157 | (0.261, 0.682) | 390 |
| | 1-429 | 3-124 | 1:3 | 5.00 | 144 | (0.261, 0.672) | 308 |
| Example | 1-439 | 2-84 | 1:1 | 4.19 | 209 | (0.271, 0.651) | 482 |
| 100 | 1-439 | 2-84 | 1:2 | 4.39 | 195 | (0.272, 0.674) | 578 |
| | 1-439 | 2-84 | 1:3 | 4.47 | 181 | (0.265, 0.683) | 417 |
| Example | 1-441 | 2-88 | 1:1 | 3.99 | 252 | (0.271, 0.651) | 37 |
| 101 | 1-441 | 2-88 | 1:2 | 4.18 | 235 | (0.272, 0.674) | 445 |
| | 1-441 | 2-88 | 1:3 | 4.26 | 218 | (0.265, 0.683) | 321 |
| Example | 1-463 | 2-106 | 1:1 | 3.90 | 212 | (0.271, 0.651) | 467 |
| 102 | 1-463 | 2-106 | 1:2 | 4.08 | 197 | (0.272, 0.674) | 560 |
| | 1-463 | 2-106 | 1:3 | 4.16 | 183 | (0.265, 0.683) | 404 |
| Example | 1-439 | 3-89 | 1:1 | 4.39 | 181 | (0.271, 0.651) | 546 |
| 103 | 1-439 | 3-89 | 1:2 | 4.79 | 167 | (0.272, 0.674) | 610 |
| | 1-439 | 3-89 | 1:3 | 4.99 | 153 | (0.265, 0.683) | 482 |
| Example | 1-441 | 3-91 | 1:1 | 4.18 | 218 | (0.271, 0.651) | 420 |
| 104 | 1-441 | 3-91 | 1:2 | 4.56 | 202 | (0.272, 0.674) | 469 |
| | 1-441 | 3-91 | 1:3 | 4.75 | 185 | (0.265, 0.683) | 371 |
| Example | 1-463 | 3-122 | 1:1 | 4.08 | 183 | (0.271, 0.651) | 529 |
| 105 | 1-463 | 3-122 | 1:2 | 4.45 | 169 | (0.272, 0.674) | 591 |
| | 1-463 | 3-122 | 1:3 | 4.64 | 155 | (0.265, 0.683) | 467 |
| Example | 1-135 | 2-83 | 1:1 | 3.98 | 213 | (0.279, 0.651) | 377 |
| 106 | 1-135 | 2-83 | 1:2 | 4.17 | 199 | (0.268, 0.653) | 452 |
| | 1-135 | 2-83 | 1:3 | 4.24 | 185 | (0.267, 0.661) | 326 |
| Example | 1-135 | 3-89 | 1:1 | 4.17 | 185 | (0.265, 0.683) | 427 |
| 107 | 1-135 | 3-89 | 1:2 | 4.55 | 170 | (0.261, 0.672) | 477 |
| | 1-135 | 3-89 | 1:3 | 4.74 | 156 | (0.272, 0.671) | 377 |
| Example | 1-138 | 2-84 | 1:1 | 3.79 | 254 | (0.276, 0.673) | 300 |
| 108 | 1-138 | 2-84 | 1:2 | 3.97 | 237 | (0.279, 0.651) | 360 |
| | 1-138 | 2-84 | 1:3 | 4.04 | 220 | (0.268, 0.653) | 260 |
| Example | 1-138 | 3-91 | 1:1 | 3.97 | 220 | (0.267, 0.661) | 340 |
| 109 | 1-138 | 3-91 | 1:2 | 4.33 | 203 | (0.265, 0.683) | 380 |
| | 1-138 | 3-91 | 1:3 | 4.51 | 186 | (0.274, 0.672) | 300 |
| Example | 1-213 | 2-88 | 1:1 | 3.90 | 210 | (0.276, 0.673) | 383 |
| 110 | 1-213 | 2-88 | 1:2 | 4.08 | 196 | (0.279, 0.651) | 459 |
| | 1-213 | 2-88 | 1:3 | 4.16 | 182 | (0.268, 0.653) | 332 |
| Example | 1-213 | 3-122 | 1:1 | 4.08 | 182 | (0.267, 0.661) | 434 |
| 111 | 1-213 | 3-122 | 1:2 | 4.45 | 168 | (0.265, 0.683) | 485 |
| | 1-213 | 3-122 | 1:3 | 4.64 | 154 | (0.274, 0.672) | 383 |
| Example | 1-216 | 2-106 | 1:1 | 3.78 | 264 | (0.264, 0.683) | 300 |
| 112 | 1-216 | 2-106 | 1:2 | 3.96 | 246 | (0.281, 0.654) | 360 |
| | 1-216 | 2-106 | 1:3 | 4.03 | 229 | (0.275, 0.648) | 260 |
| Example | 1-216 | 3-89 | 1:1 | 3.96 | 229 | (0.282, 0.652) | 340 |
| 113 | 1-216 | 3-89 | 1:2 | 4.32 | 211 | (0.271, 0.651) | 380 |
| | 1-216 | 3-89 | 1:3 | 4.50 | 194 | (0.267, 0.661) | 300 |
| Example | 1-342 | 2-83 | 1:1 | 3.79 | 272 | (0.265, 0.683) | 299 |
| 114 | 1-342 | 2-83 | 1:2 | 3.97 | 253 | (0.261, 0.672) | 358 |
| | 1-342 | 2-83 | 1:3 | 4.04 | 235 | (0.272, 0.671) | 259 |
| Example | 1-342 | 3-91 | 1:1 | 3.97 | 235 | (0.276, 0.673) | 338 |
| 115 | 1-342 | 3-91 | 1:2 | 4.33 | 217 | (0.279, 0.651) | 378 |
| | 1-342 | 3-91 | 1:3 | 4.51 | 199 | (0.268, 0.653) | 299 |
| Example | 1-387 | 2-84 | 1:1 | 3.89 | 209 | (0.265, 0.683) | 363 |
| 116 | 1-387 | 2-84 | 1:2 | 4.07 | 195 | (0.271, 0.651) | 436 |
| | 1-387 | 2-84 | 1:3 | 4.14 | 181 | (0.272, 0.674) | 315 |
| Example | 1-387 | 3-122 | 1:1 | 4.07 | 181 | (0.281, 0.654) | 411 |
| 117 | 1-387 | 3-122 | 1:2 | 4.44 | 167 | (0.275, 0.648) | 460 |
| | 1-387 | 3-122 | 1:3 | 4.63 | 153 | (0.282, 0.652) | 363 |

As can be seen from the results of Table 8, Examples 52 to 117, which are organic light-emitting devices using the compound represented by Chemical Formula 1 of the present invention and the compound of Chemical Formula 3 or 4 as a light-emitting layer material, have a lower driving voltage, and significantly improved luminous efficiency and lifespan compared to Comparative Examples 19 to 22, which are organic light-emitting devices using A, I, and K compounds and the compound of Chemical Formula 3 or 4 of the present invention as a light-emitting layer material.

This result is due to an exciplex generated when two kinds of compounds are comprised simultaneously.

The exciplex phenomenon is a phenomenon of emitting energy having a size of a HOMO level of a donor (p-host) and a LUMO level of an acceptor (n-host) due to electron exchange between two molecules. When a donor (p-host) having good hole transport capability and an acceptor (n-host) having good electron transport capability are used as hosts of the light-emitting layer, holes are injected into the p-host and electrons are injected into the n-host, so that a driving voltage may be lowered, thereby helping to improve a lifespan.

From the results of Table 8, when the compound represented by Chemical Formula 1 of the present invention and the compound of Chemical Formula 3 or 4 are comprised in a specific ratio in the light-emitting layer of the organic light-emitting device, it can be confirmed that optimal efficiency or lifespan characteristics are exhibited as charge balance between holes and electrons in the light-emitting layer is achieved when the device is driven. Conversely, when a ratio between the compound represented by Chemical Formula 1 and the compound represented by Chemical Formula 3 or 4 deviates enough to cause charge imbalance between holes and electrons in the light-emitting layer when the device is driven, it may be inappropriate to achieve desired efficiency or lifespan characteristics of the device.

Specifically, the donor role is performed by the compound of Chemical Formula 3 or 4, and the acceptor role is performed by the heterocyclic compound of Chemical Formula 1, whereas in Comparative Examples 19 to 22, even if the donor is the same, since a compound exhibiting poor device characteristics as a light-emitting layer host is used as an acceptor as examined in Table 8, it can be confirmed that a remarkable difference in device characteristics between the Example group and the Comparative Example group appears.

### REFERENCE NUMERALS

100: Substrate
200: Anode
300: Organic material layer
301: Hole injection layer
302: Hole transport layer
303: Light-emitting layer
304: Electron transport layer
305: Electron injection layer
306: Electron blocking layer
307: Hole blocking layer
400: Cathode

What is claimed is:

1. A heterocyclic compound represented by the following Chemical Formula 1: 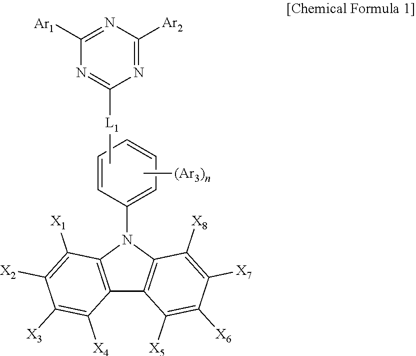
wherein, in Chemical Formula 1,
Ar₁ and Ar₂ are the same as or different from each other, and are each independently a substituted or unsubstituted C6 to C60 aryl group; or a substituted or unsubstituted C2 to C60 heteroaryl group;
L₁ is a direct bond; or a substituted or unsubstituted C6 to C60 arylene group;
Ar₃ is hydrogen; deuterium; a substituted or unsubstituted C6 to C60 aryl group; or a substituted or unsubstituted C2 to C60 heteroaryl group,
n is an integer from 1 to 4, and when n is 2 or greater, substituents in parentheses are the same as or different from each other;
when L₁ is a direct bond, Ar₃ is a substituted or unsubstituted C6 to C60 aryl group; or a substituted or unsubstituted C2 to C60 heteroaryl group;
X₁ to X₅ and X₇ are the same as or different from each other, and are each independently hydrogen; deuterium; or a substituted or unsubstituted C6 to C60 aryl group;
any one of X₆ and X₈ is represented by the following Chemical Formula 2, and the remainder is hydrogen; or deuterium; 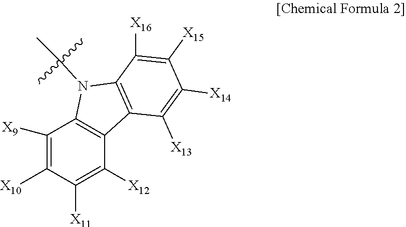
wherein, in Chemical Formula 2,
X₉ to X₁₆ are the same as or different from each other, and are each independently hydrogen; deuterium; or a substituted or unsubstituted C6 to C60 aryl group; 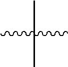
represents a site bonded to X₆ or X₈ of Chemical Formula 1; and
at least six of X₁ to X₁₆ are substituted with deuterium.

2. The heterocyclic compound according to claim 1, wherein Chemical Formula 1 is represented by any one of the following Chemical Formulas 1-1 to 1-3: 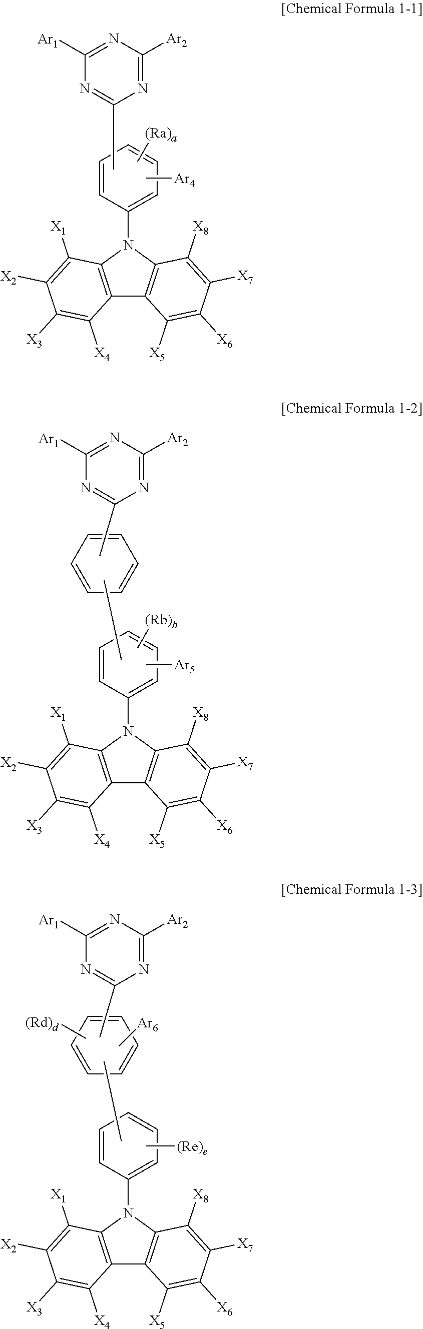
wherein, in Chemical Formulas 1-1 to 1-3,
definitions of Ar₁, Ar₂, and X₁ to X₈ are the same as the definitions in Chemical Formula 1;
Ar₄ to Ar₆ are each a substituted or unsubstituted C6 to C60 aryl group; or a substituted or unsubstituted C2 to C60 heteroaryl group; and
Ra, Rb, Rc, Rd, and Re are each independently hydrogen, deuterium, a substituted or unsubstituted C6 to C60 aryl group, or a substituted or unsubstituted C2 to C60 heteroaryl group;
a, c, and d are each an integer from 0 to 3, and when a, c, and d are each 2 or greater, substituents in parentheses are the same as or different from each other; and
b and e are each an integer from 0 to 4, and when b and e are each 2 or greater, substituents in parentheses are the same as or different from each other.

3. The heterocyclic compound according to claim 1, wherein Ar₁ and Ar₂ are the same as or different from each other, and are each independently a substituted or unsubstituted C6 to C20 aryl group, or a substituted or unsubstituted C2 to C20 heteroaryl group.

4. The heterocyclic compound according to claim 1, wherein L₁ is a direct bond, or a substituted or unsubstituted C6 to C20 arylene group.

5. The heterocyclic compound according to claim 1, wherein Ar₃ is hydrogen, deuterium, a substituted or unsubstituted C6 to C20 aryl group, or a substituted or unsubstituted C2 to C20 heteroaryl group.

6. The heterocyclic compound according to claim 1, wherein Chemical Formula 1 is represented by any one of the following compounds:

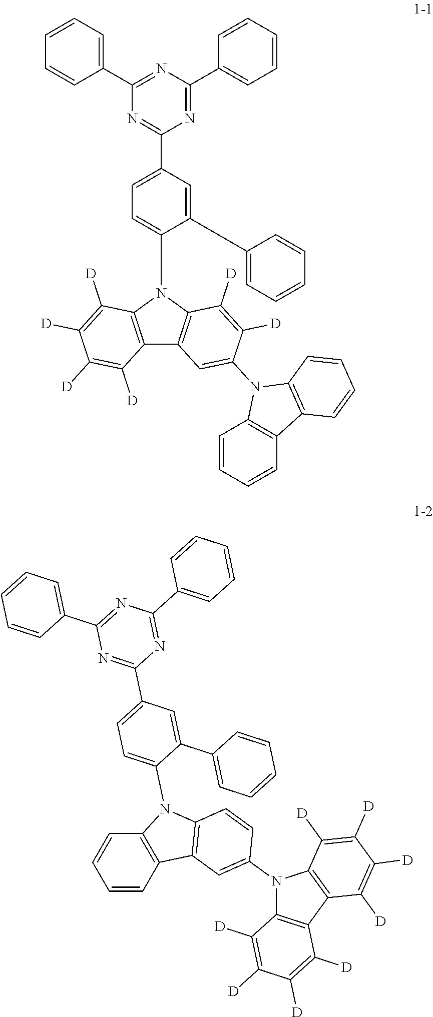 [SMILES: [2H]c1c([2H])c([2H])c2c(c1[2H])c1cc(-n3c4ccccc4c4ccccc43)c([2H])c([2H])c1n2-c1ccc(-c2nc(-c3ccccc3)nc(-c3ccccc3)n2)cc1-c1ccccc1 ; [2H]c1c([2H])c([2H])c2c(c1[2H])c1c([2H])c([2H])c([2H])c([2H])c1n2-c1ccc2c(c1)c1ccccc1n2-c1ccc(-c2nc(-c3ccccc3)nc(-c3ccccc3)n2)cc1-c1ccccc1]

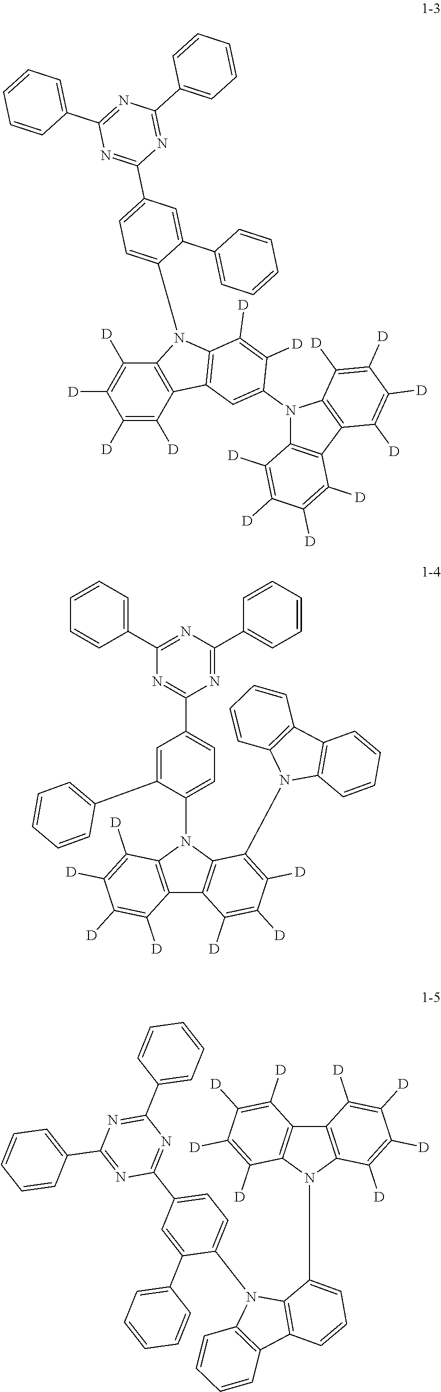 [SMILES: [2H]c1c([2H])c([2H])c2c(c1[2H])c1cc(-n3c4c([2H])c([2H])c([2H])c([2H])c4c4c([2H])c([2H])c([2H])c([2H])c43)c([2H])c([2H])c1n2-c1ccc(-c2nc(-c3ccccc3)nc(-c3ccccc3)n2)cc1-c1ccccc1 ; [2H]c1c([2H])c([2H])c2c(c1[2H])c1c([2H])c([2H])c([2H])c(-n3c4ccccc4c4ccccc43)c1n2-c1ccc(-c2nc(-c3ccccc3)nc(-c3ccccc3)n2)cc1-c1ccccc1 ; [2H]c1c([2H])c([2H])c2c(c1[2H])c1c([2H])c([2H])c([2H])c([2H])c1n2-c1cccc2c3ccccc3n(-c3ccc(-c4nc(-c5ccccc5)nc(-c5ccccc5)n4)cc3-c3ccccc3)c12]

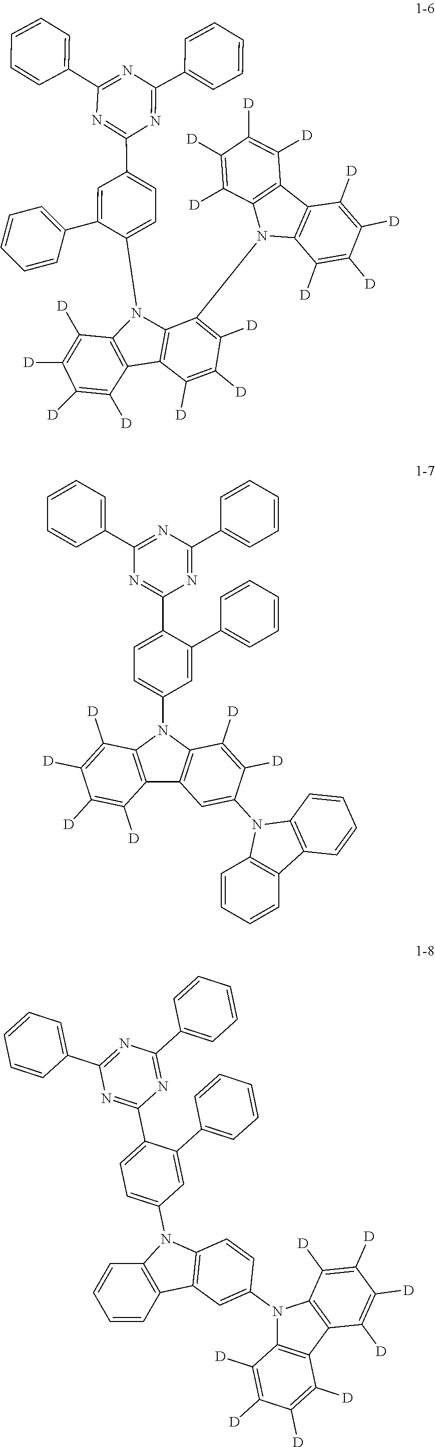 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2nc(-c3ccccc3)nc(-c3ccccc3)n2)cc1-c1ccccc1 ; [2H]c1c([2H])c([2H])c2c(c1[2H])c1cc(-n3c4ccccc4c4ccccc43)c([2H])c([2H])c1n2-c1ccc(-c2nc(-c3ccccc3)nc(-c3ccccc3)n2)c(-c2ccccc2)c1 ; [2H]c1c([2H])c([2H])c2c(c1[2H])c1c([2H])c([2H])c([2H])c([2H])c1n2-c1ccc2c(c1)c1ccccc1n2-c1ccc(-c2nc(-c3ccccc3)nc(-c3ccccc3)n2)c(-c2ccccc2)c1]

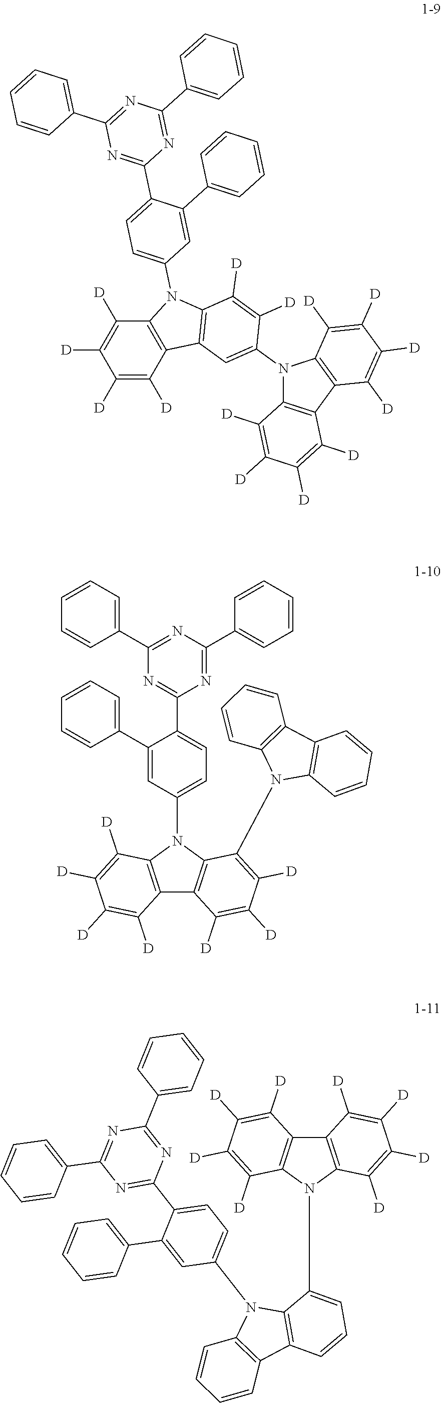 [SMILES: [2H]c1c([2H])c([2H])c2c(c1[2H])c1cc(-n3c4c([2H])c([2H])c([2H])c([2H])c4c4c([2H])c([2H])c([2H])c([2H])c43)c([2H])c([2H])c1n2-c1ccc(-c2nc(-c3ccccc3)nc(-c3ccccc3)n2)c(-c2ccccc2)c1 ; [2H]c1c([2H])c([2H])c2c(c1[2H])c1c([2H])c([2H])c([2H])c(-n3c4ccccc4c4ccccc43)c1n2-c1ccc(-c2nc(-c3ccccc3)nc(-c3ccccc3)n2)c(-c2ccccc2)c1 ; [2H]c1c([2H])c([2H])c2c(c1[2H])c1c([2H])c([2H])c([2H])c([2H])c1n2-c1cccc2c3ccccc3n(-c3ccc(-c4nc(-c5ccccc5)nc(-c5ccccc5)n4)c(-c4ccccc4)c3)c12]

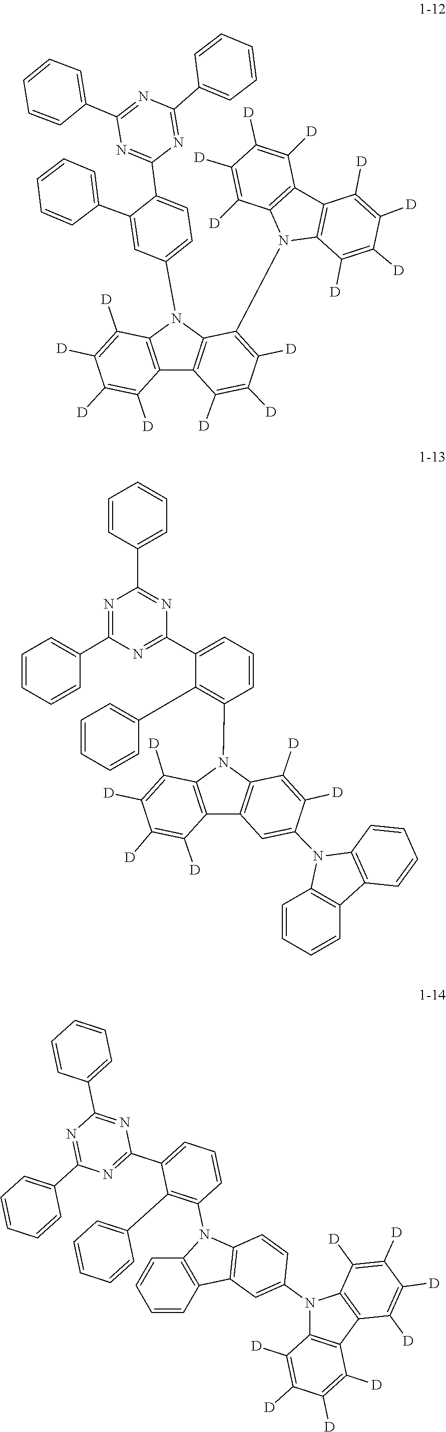 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2nc(-c3ccccc3)nc(-c3ccccc3)n2)c(-c2ccccc2)c1 ; [2H]c1c([2H])c([2H])c2c(c1[2H])c1cc(-n3c4ccccc4c4ccccc43)c([2H])c([2H])c1n2-c1cccc(-c2nc(-c3ccccc3)nc(-c3ccccc3)n2)c1-c1ccccc1 ; [2H]c1c([2H])c([2H])c2c(c1[2H])c1c([2H])c([2H])c([2H])c([2H])c1n2-c1ccc2c(c1)c1ccccc1n2-c1cccc(-c2nc(-c3ccccc3)nc(-c3ccccc3)n2)c1-c1ccccc1]

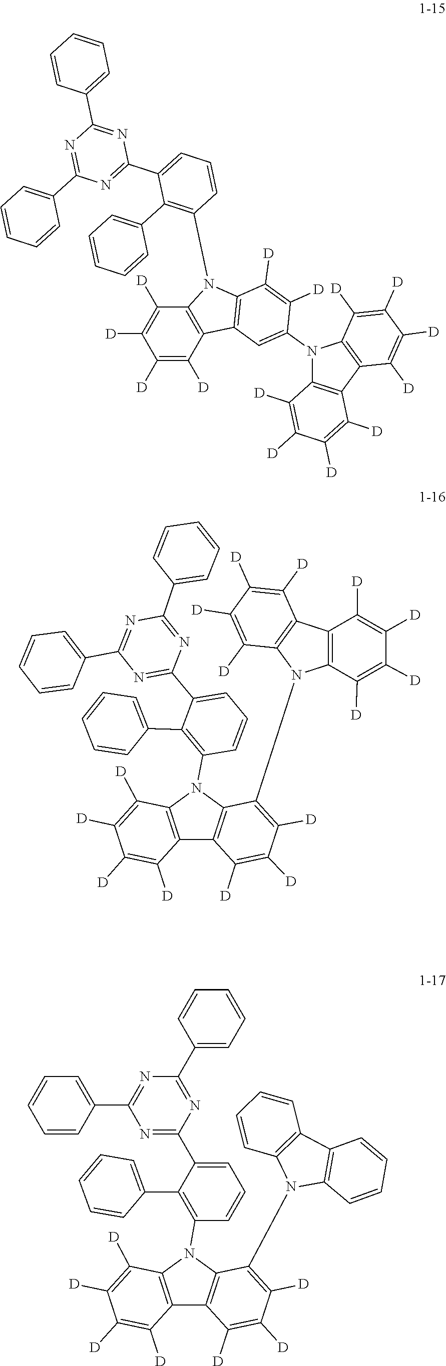 [SMILES: [2H]c1c([2H])c([2H])c2c(c1[2H])c1cc(-n3c4c([2H])c([2H])c([2H])c([2H])c4c4c([2H])c([2H])c([2H])c([2H])c43)c([2H])c([2H])c1n2-c1cccc(-c2nc(-c3ccccc3)nc(-c3ccccc3)n2)c1-c1ccccc1 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2nc(-c3ccccc3)nc(-c3ccccc3)n2)c1-c1ccccc1 ; [2H]c1c([2H])c([2H])c2c(c1[2H])c1c([2H])c([2H])c([2H])c(-n3c4ccccc4c4ccccc43)c1n2-c1cccc(-c2nc(-c3ccccc3)nc(-c3ccccc3)n2)c1-c1ccccc1]

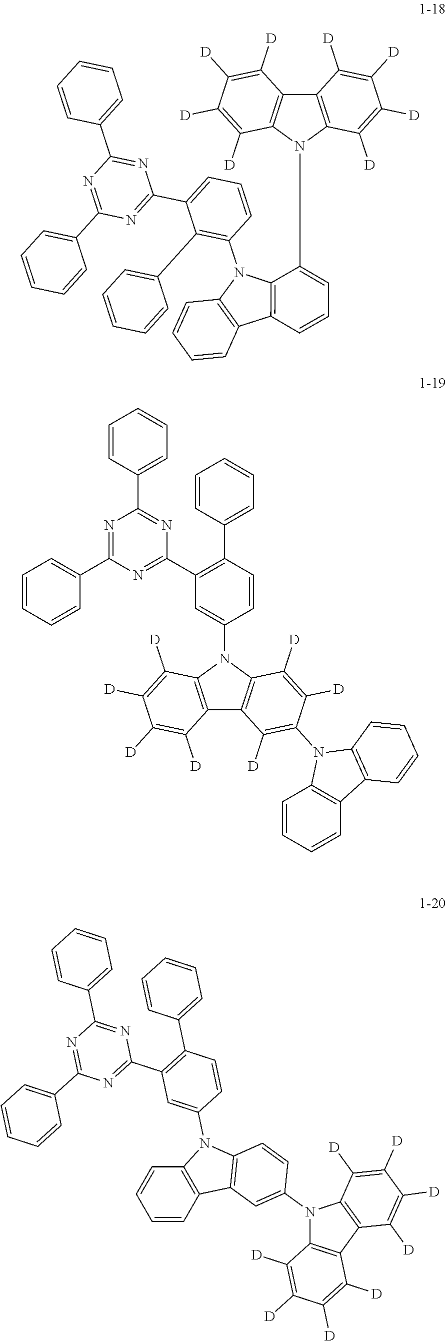 [SMILES: [2H]c1c([2H])c([2H])c2c(c1[2H])c1c([2H])c([2H])c([2H])c([2H])c1n2-c1cccc2c3ccccc3n(-c3cccc(-c4nc(-c5ccccc5)nc(-c5ccccc5)n4)c3-c3ccccc3)c12 ; [2H]c1c([2H])c([2H])c2c(c1[2H])c1c([2H])c(-n3c4ccccc4c4ccccc43)c([2H])c([2H])c1n2-c1ccc(-c2ccccc2)c(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1ccc(-c2ccccc2)c(-c2nc(-c3ccccc3)nc(-c3ccccc3)n2)c1]

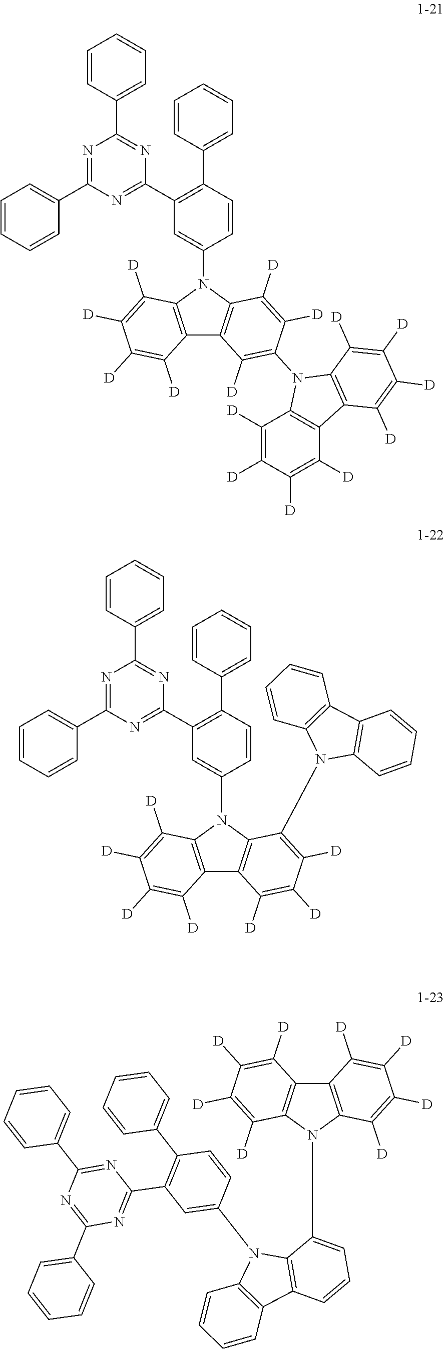 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)c(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1ccc(-c2ccccc2)c(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3ccc(-c4ccccc4)c(-c4nc(-c5ccccc5)nc(-c5ccccc5)n4)c3)c12]

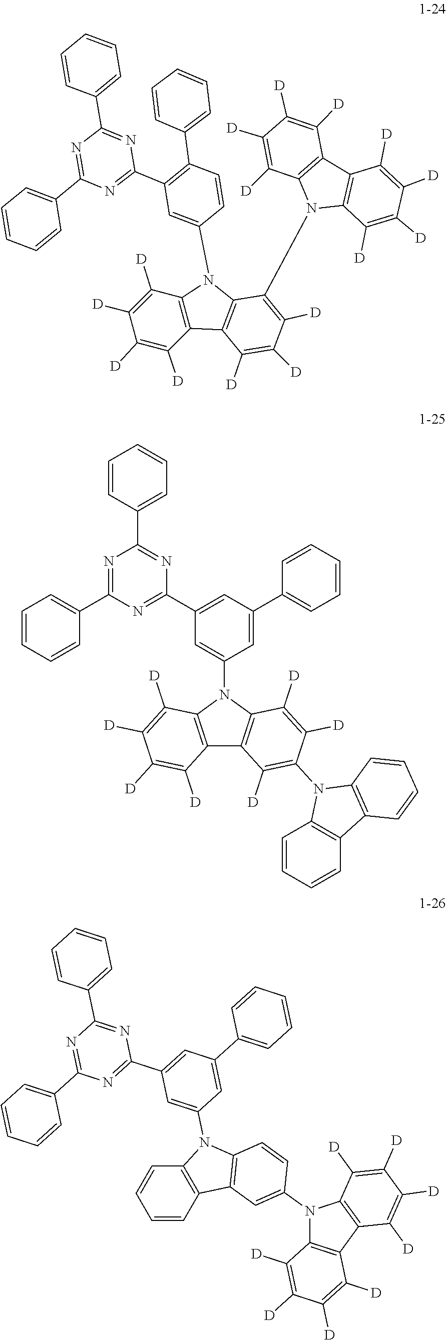 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)c(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cc(-c2ccccc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2)cc(-c2nc(-c3ccccc3)nc(-c3ccccc3)n2)c1]

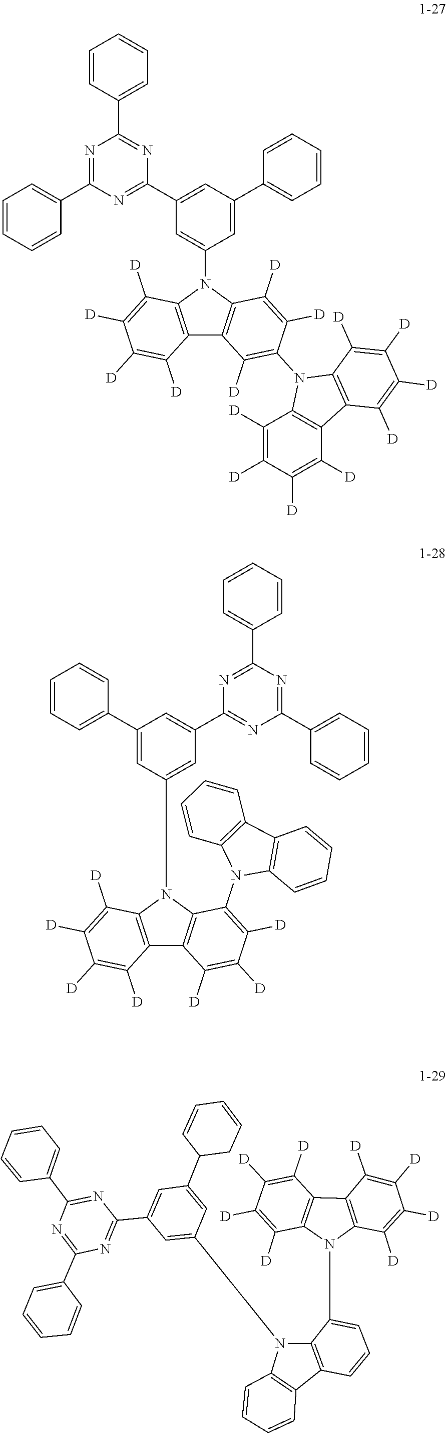 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)cc(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cc(-c2ccccc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3cc(-c4nc(-c5ccccc5)nc(-c5ccccc5)n4)cc(C4C=CC=CC4)c3)c12]

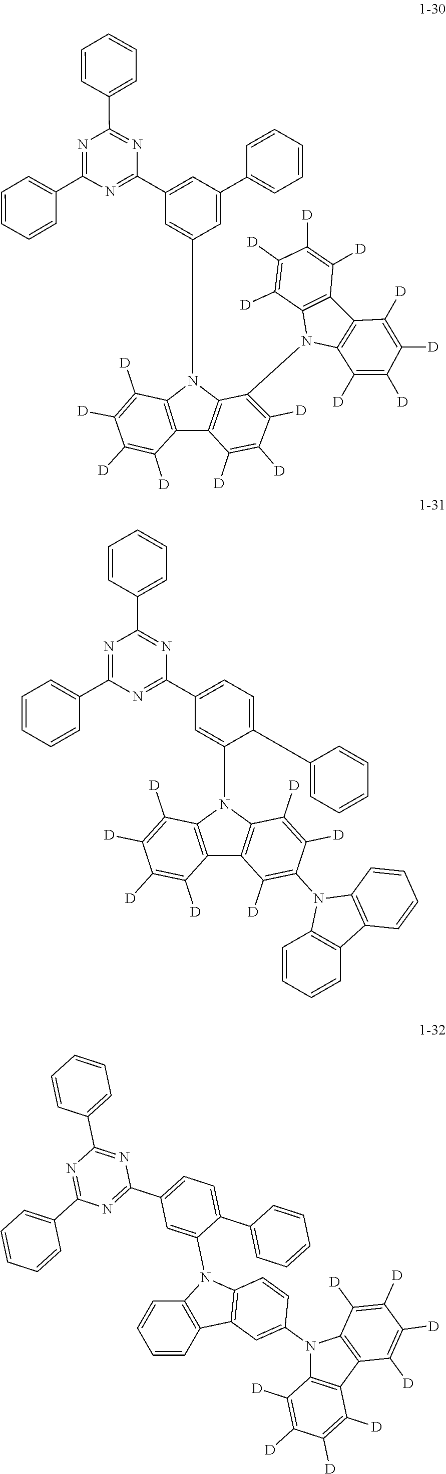 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2)cc(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cc(-c2nc(-c3ccccc3)nc(-c3ccccc3)n2)ccc1-c1ccccc1 ; [2H]c1c([2H])c([2H])c2c(c1[2H])c1c([2H])c([2H])c([2H])c([2H])c1n2-c1ccc2c(c1)c1ccccc1n2-c1cc(-c2nc(-c3ccccc3)nc(-c3ccccc3)n2)ccc1-c1ccccc1]

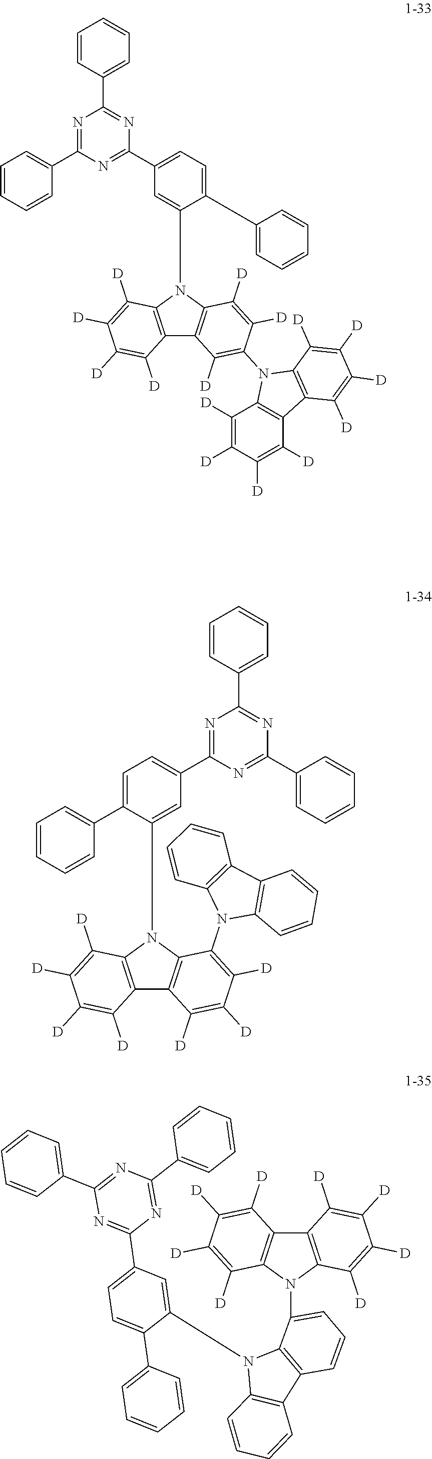 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2nc(-c3ccccc3)nc(-c3ccccc3)n2)ccc1-c1ccccc1 ; [2H]c1c([2H])c([2H])c2c(c1[2H])c1c([2H])c([2H])c([2H])c(-n3c4ccccc4c4ccccc43)c1n2-c1cc(-c2nc(-c3ccccc3)nc(-c3ccccc3)n2)ccc1-c1ccccc1 ; [2H]c1c([2H])c([2H])c2c(c1[2H])c1c([2H])c([2H])c([2H])c([2H])c1n2-c1cccc2c3ccccc3n(-c3cc(-c4nc(-c5ccccc5)nc(-c5ccccc5)n4)ccc3-c3ccccc3)c12]

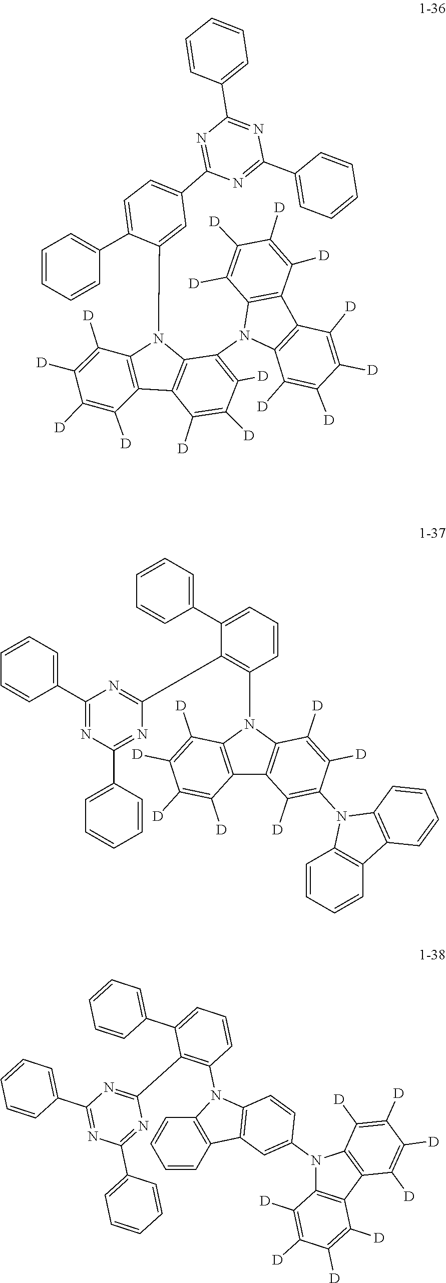 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2nc(-c3ccccc3)nc(-c3ccccc3)n2)ccc1-c1ccccc1 ; [2H]c1c([2H])c([2H])c2c(c1[2H])c1c([2H])c(-n3c4ccccc4c4ccccc43)c([2H])c([2H])c1n2-c1cccc(-c2ccccc2)c1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1cccc(-c2ccccc2)c1-c1nc(-c2ccccc2)nc(-c2ccccc2)n1]

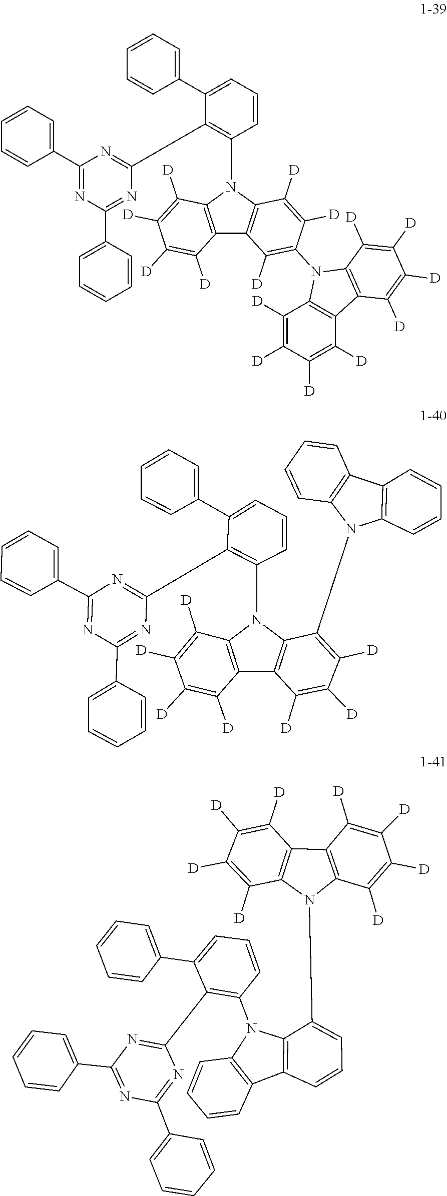 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccccc2)c1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1cccc(-c2ccccc2)c1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3cccc(-c4ccccc4)c3-c3nc(-c4ccccc4)nc(-c4ccccc4)n3)c12]

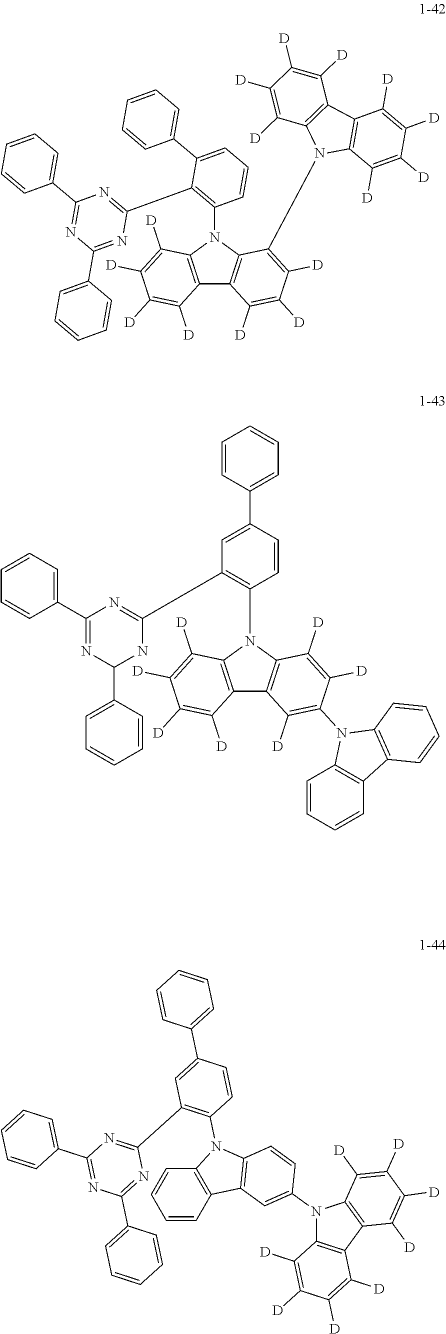

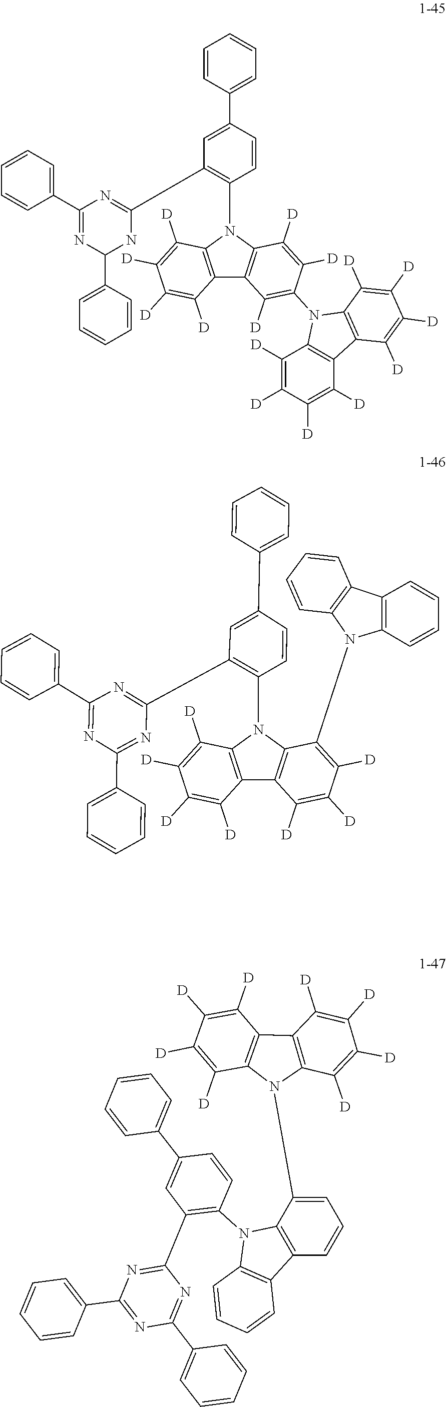

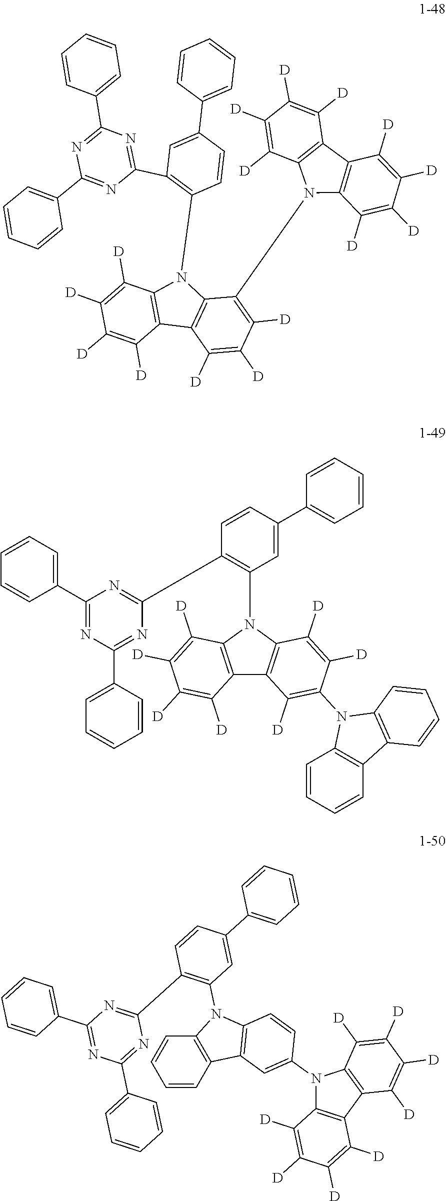 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)cc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1cc(-c2ccccc2)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2)ccc1-c1nc(-c2ccccc2)nc(-c2ccccc2)n1]

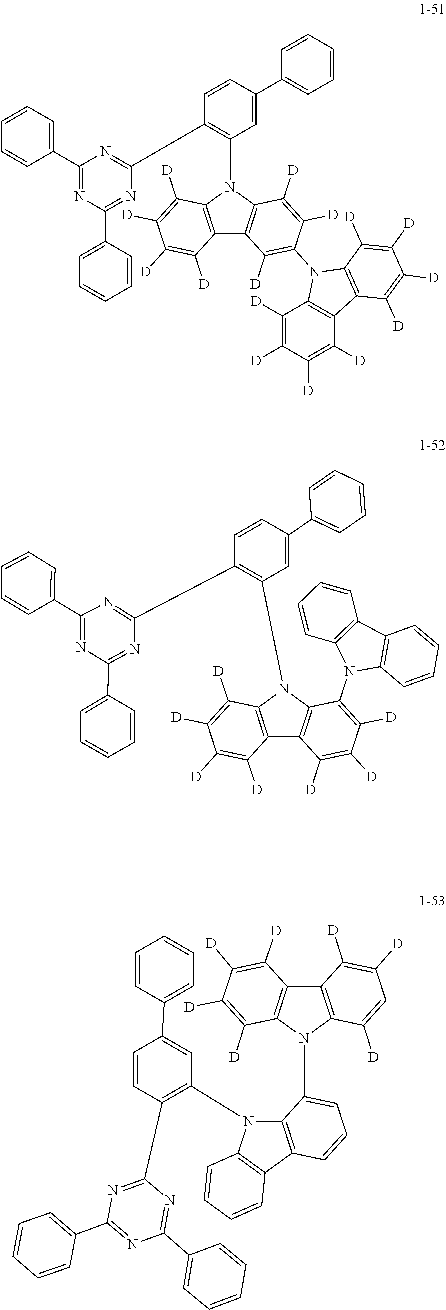 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)ccc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1cc(-c2ccccc2)ccc1-c1nc(-c2ccccc2)nc(-c2ccccc2)n1 ; [2H]c1cc([2H])c2c(c1[2H])c1c([2H])c([2H])c([2H])c([2H])c1n2-c1cccc2c3ccccc3n(-c3cc(-c4ccccc4)ccc3-c3nc(-c4ccccc4)nc(-c4ccccc4)n3)c12]

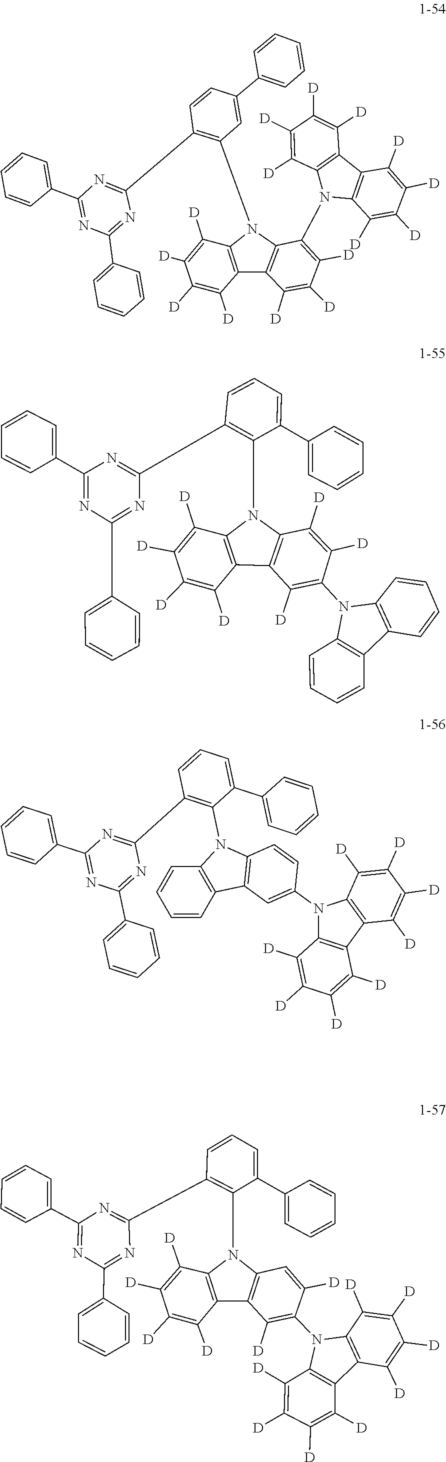

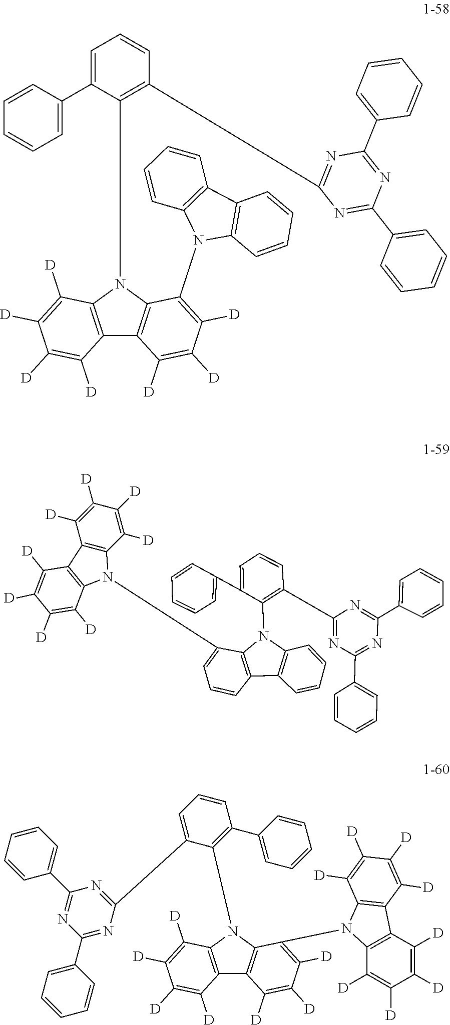 [SMILES: [2H]c1c([2H])c([2H])c2c(c1[2H])c1c([2H])c([2H])c([2H])c(-n3c4ccccc4c4ccccc43)c1n2-c1c(-c2ccccc2)cccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3c(-c4ccccc4)cccc3-c3nc(-c4ccccc4)nc(-c4ccccc4)n3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1c(-c2ccccc2)cccc1-c1nc(-c2ccccc2)nc(-c2ccccc2)n1]

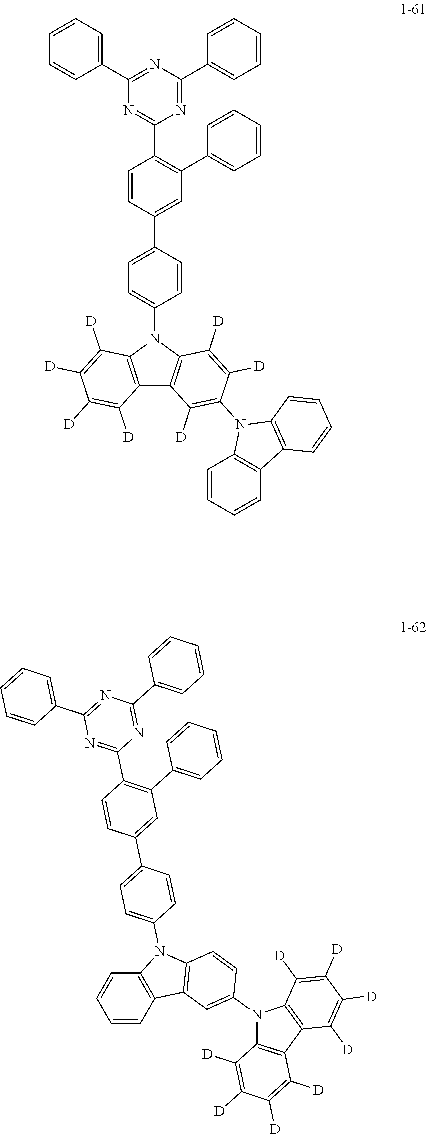 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3nc(-c4ccccc4)nc(-c4ccccc4)n3)c(-c3ccccc3)c2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3nc(-c4ccccc4)nc(-c4ccccc4)n3)c(-c3ccccc3)c2)cc1]

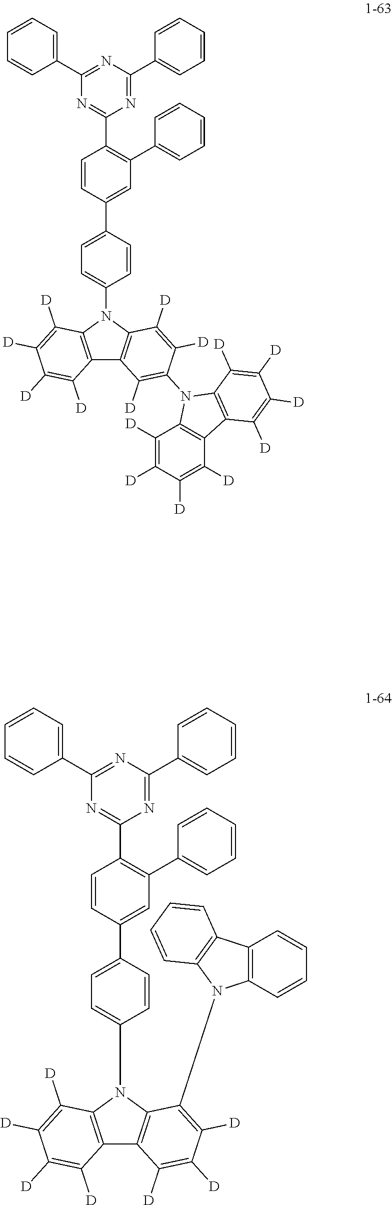 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)c(-c3ccccc3)c2)cc1 ; [2H]c1c([2H])c([2H])c2c(c1[2H])c1c([2H])c([2H])c([2H])c(-n3c4ccccc4c4ccccc43)c1n2-c1ccc(-c2ccc(-c3nc(-c4ccccc4)nc(-c4ccccc4)n3)c(-c3ccccc3)c2)cc1]

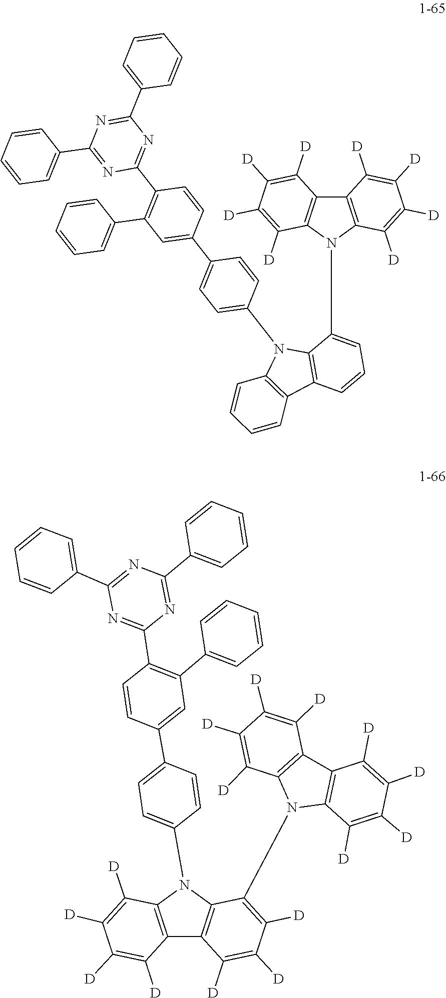 [SMILES: [2H]c1c([2H])c([2H])c2c(c1[2H])c1c([2H])c([2H])c([2H])c([2H])c1n2-c1cccc2c3ccccc3n(-c3ccc(-c4ccc(-c5nc(-c6ccccc6)nc(-c6ccccc6)n5)c(-c5ccccc5)c4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)c(-c3ccccc3)c2)cc1]

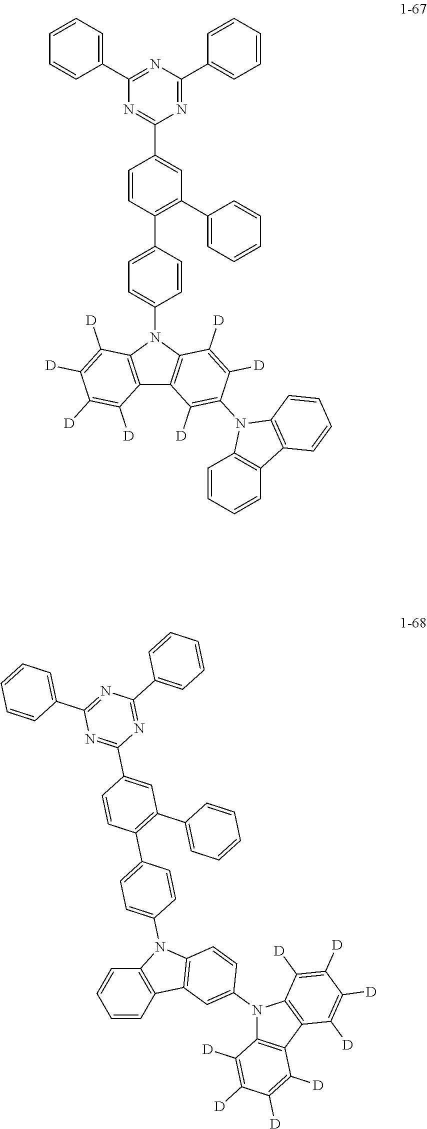 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3nc(-c4ccccc4)nc(-c4ccccc4)n3)cc2-c2ccccc2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3nc(-c4ccccc4)nc(-c4ccccc4)n3)cc2-c2ccccc2)cc1]

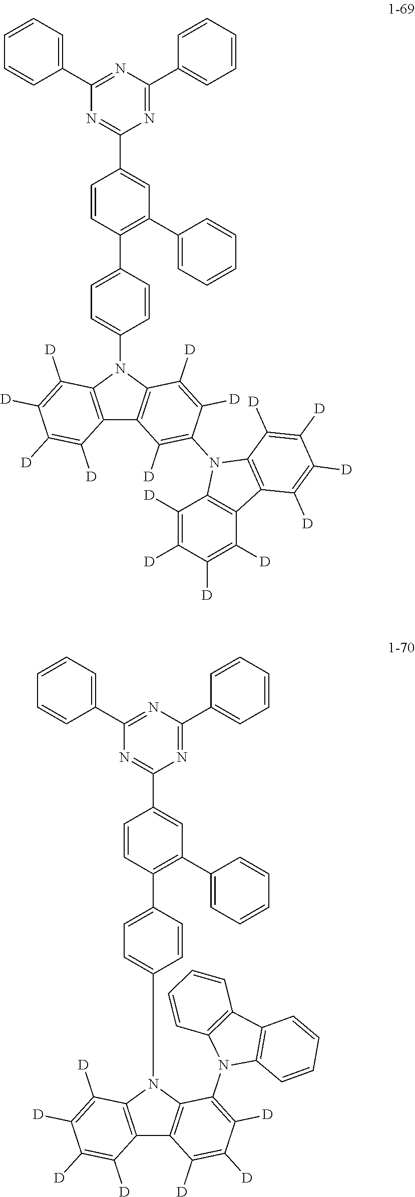 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2-c2ccccc2)cc1 ; [2H]c1c([2H])c([2H])c2c(c1[2H])c1c([2H])c([2H])c([2H])c(-n3c4ccccc4c4ccccc43)c1n2-c1ccc(-c2ccc(-c3nc(-c4ccccc4)nc(-c4ccccc4)n3)cc2-c2ccccc2)cc1]

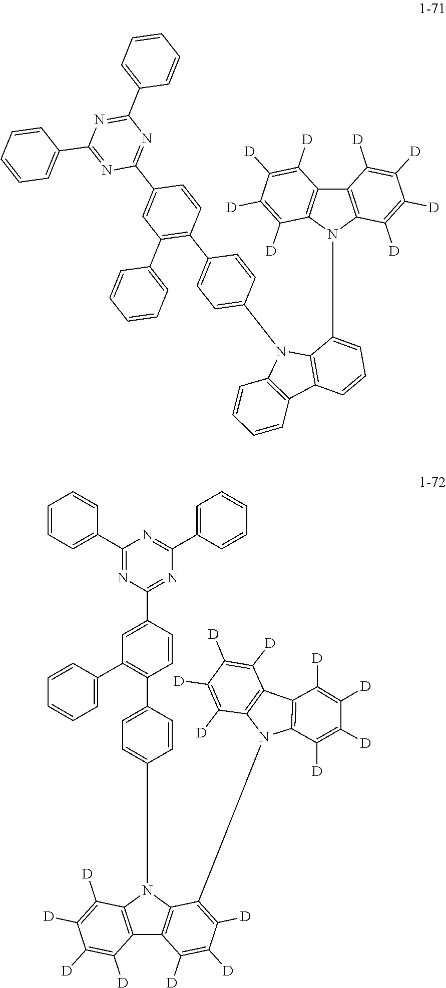 [SMILES: [2H]c1c([2H])c([2H])c2c(c1[2H])c1c([2H])c([2H])c([2H])c([2H])c1n2-c1cccc2c3ccccc3n(-c3ccc(-c4ccc(-c5nc(-c6ccccc6)nc(-c6ccccc6)n5)cc4-c4ccccc4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)cc2-c2ccccc2)cc1]

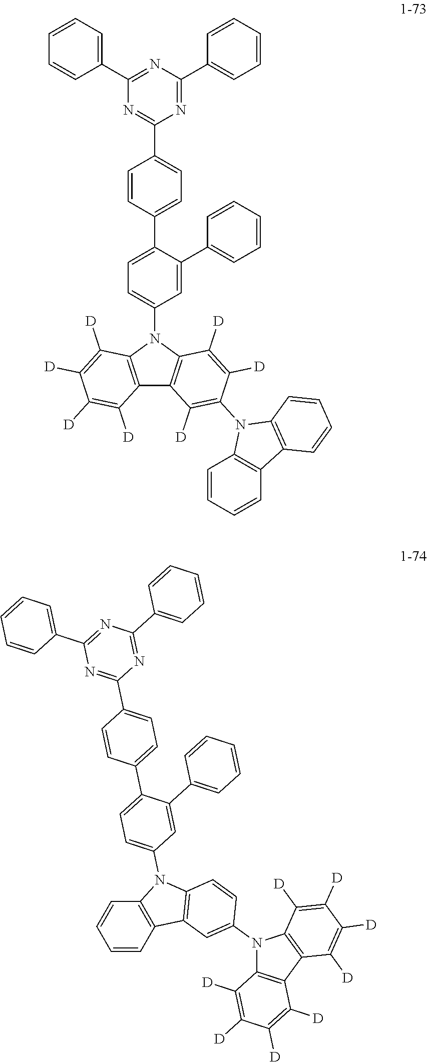 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3nc(-c4ccccc4)nc(-c4ccccc4)n3)cc2)c(-c2ccccc2)c1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3nc(-c4ccccc4)nc(-c4ccccc4)n3)cc2)c(-c2ccccc2)c1]

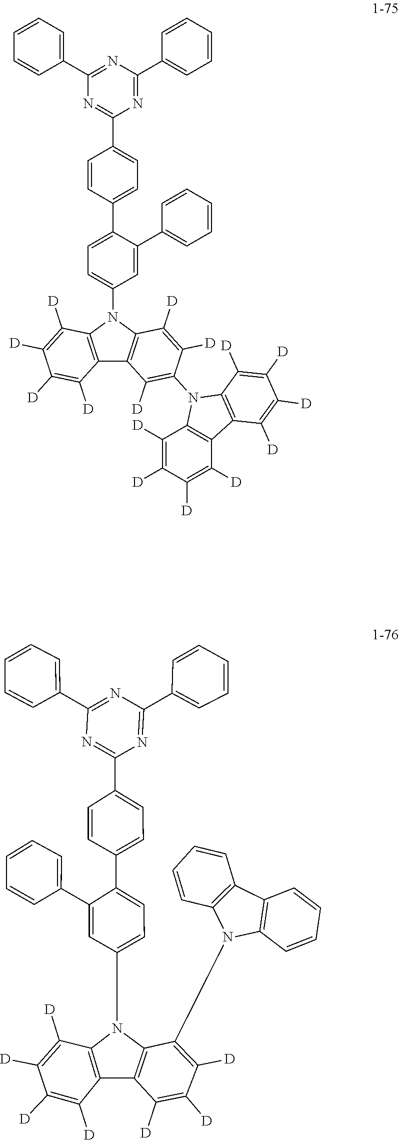 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)c(-c2ccccc2)c1 ; [2H]c1c([2H])c([2H])c2c(c1[2H])c1c([2H])c([2H])c([2H])c(-n3c4ccccc4c4ccccc43)c1n2-c1ccc(-c2ccc(-c3nc(-c4ccccc4)nc(-c4ccccc4)n3)cc2)c(-c2ccccc2)c1]

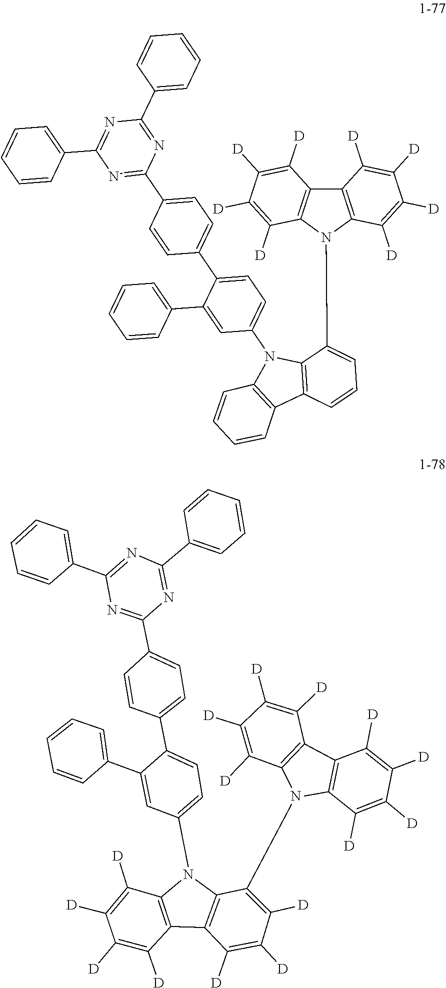

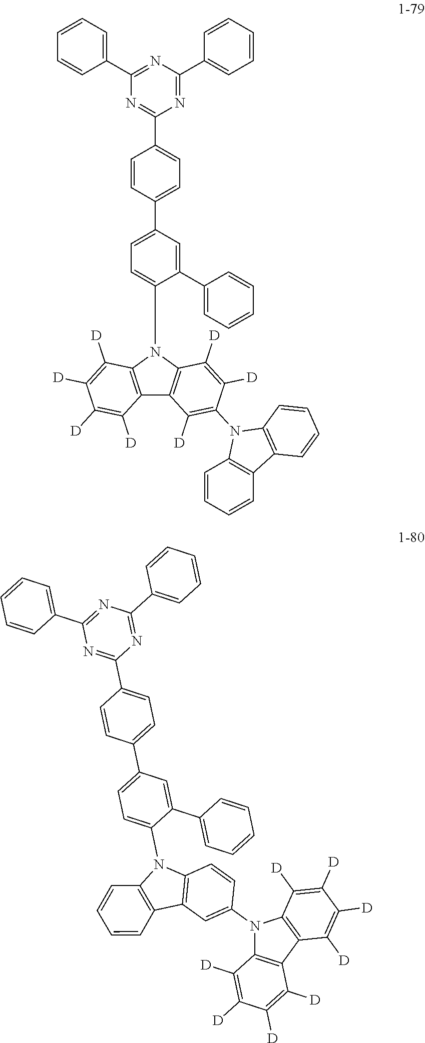 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3nc(-c4ccccc4)nc(-c4ccccc4)n3)cc2)cc1-c1ccccc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3nc(-c4ccccc4)nc(-c4ccccc4)n3)cc2)cc1-c1ccccc1]

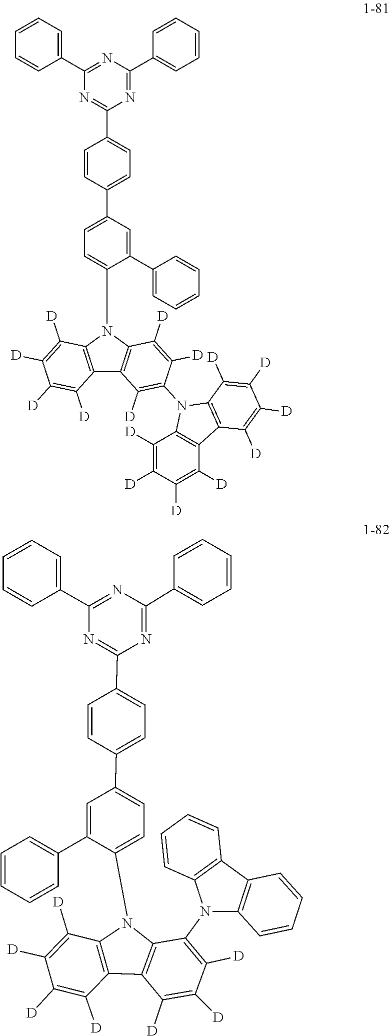 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)cc1-c1ccccc1 ; [2H]c1c([2H])c([2H])c2c(c1[2H])c1c([2H])c([2H])c([2H])c(-n3c4ccccc4c4ccccc43)c1n2-c1ccc(-c2ccc(-c3nc(-c4ccccc4)nc(-c4ccccc4)n3)cc2)cc1-c1ccccc1]

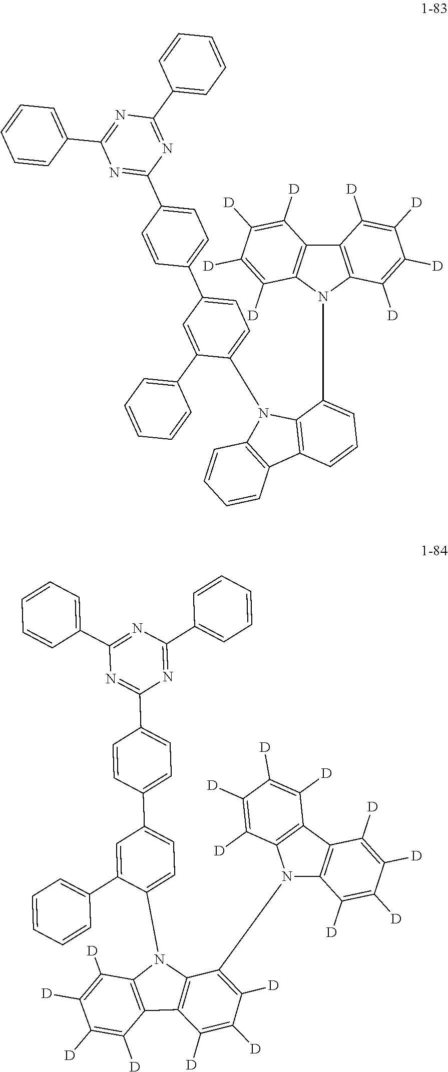 [SMILES: [2H]c1c([2H])c([2H])c2c(c1[2H])c1c([2H])c([2H])c([2H])c([2H])c1n2-c1cccc2c3ccccc3n(-c3ccc(-c4ccc(-c5nc(-c6ccccc6)nc(-c6ccccc6)n5)cc4)cc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)cc2)cc1-c1ccccc1]

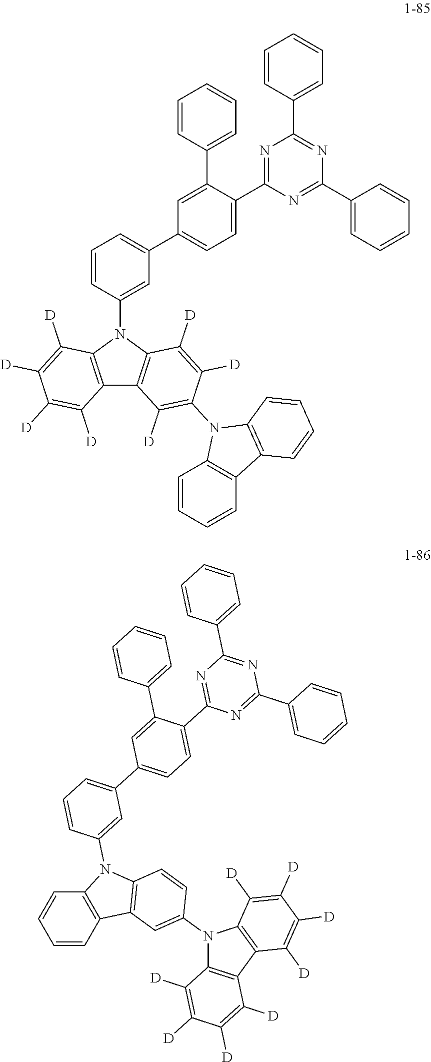

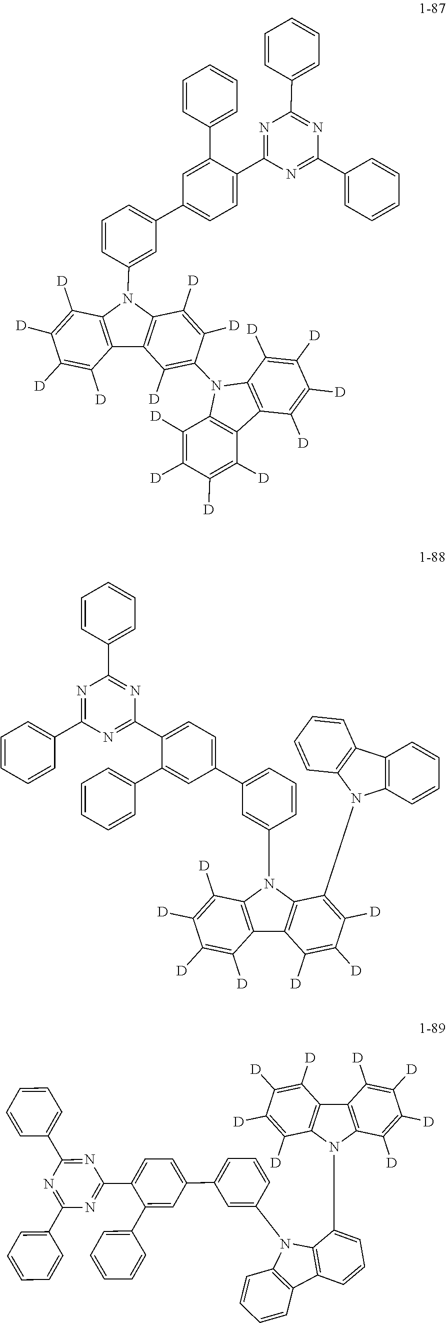 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccc(-c3nc(-c4ccccc4)nc(-c4ccccc4)n3)c(-c3ccccc3)c2)c1 ; [2H]c1c([2H])c([2H])c2c(c1[2H])c1c([2H])c([2H])c([2H])c(-n3c4ccccc4c4ccccc43)c1n2-c1cccc(-c2ccc(-c3nc(-c4ccccc4)nc(-c4ccccc4)n3)c(-c3ccccc3)c2)c1 ; [2H]c1c([2H])c([2H])c2c(c1[2H])c1c([2H])c([2H])c([2H])c([2H])c1n2-c1cccc2c3ccccc3n(-c3cccc(-c4ccc(-c5nc(-c6ccccc6)nc(-c6ccccc6)n5)c(-c5ccccc5)c4)c3)c12]

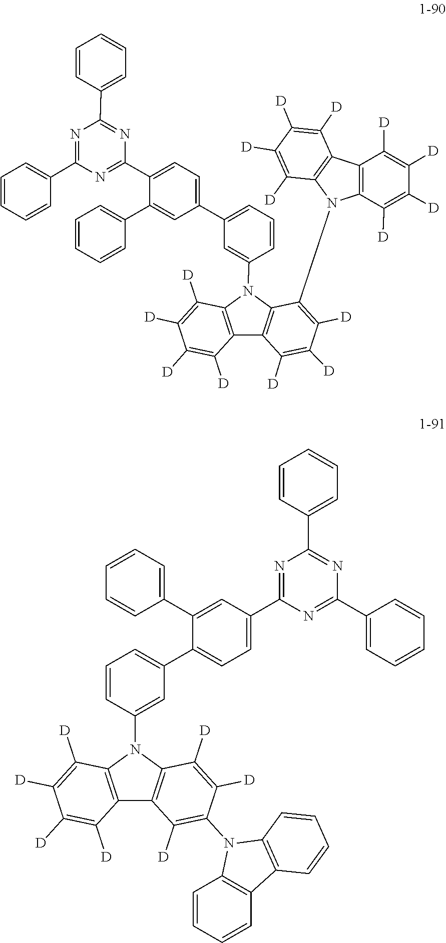 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccc(-c3nc(-c4ccccc4)nc(-c4ccccc4)n3)c(-c3ccccc3)c2)c1 ; [2H]c1c([2H])c([2H])c2c(c1[2H])c1c([2H])c(-n3c4ccccc4c4ccccc43)c([2H])c([2H])c1n2-c1cccc(-c2ccc(-c3nc(-c4ccccc4)nc(-c4ccccc4)n3)cc2-c2ccccc2)c1]

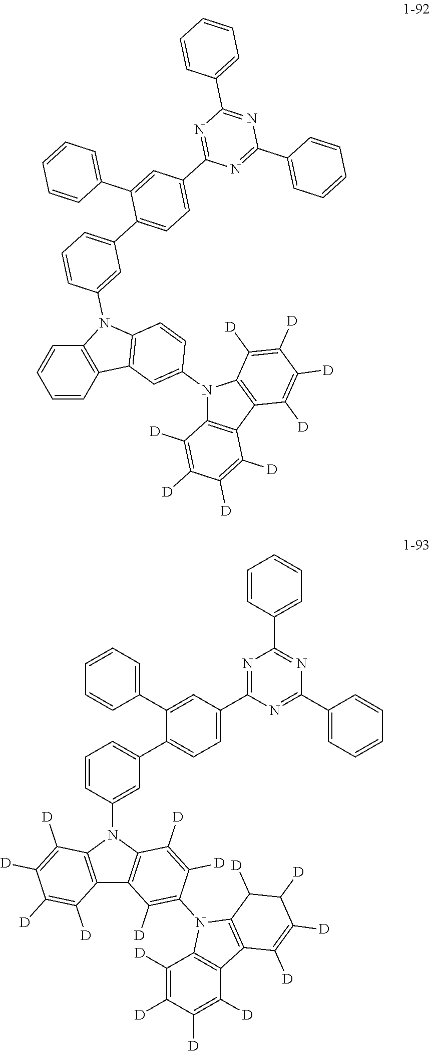 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2ccc(-c3nc(-c4ccccc4)nc(-c4ccccc4)n3)cc2-c2ccccc2)c1 ; [2H]C1=C([2H])C([2H])C([2H])c2c1c1c([2H])c([2H])c([2H])c([2H])c1n2-c1c([2H])c([2H])c2c(c1[2H])c1c([2H])c([2H])c([2H])c([2H])c1n2-c1cccc(-c2ccc(-c3nc(-c4ccccc4)nc(-c4ccccc4)n3)cc2-c2ccccc2)c1]

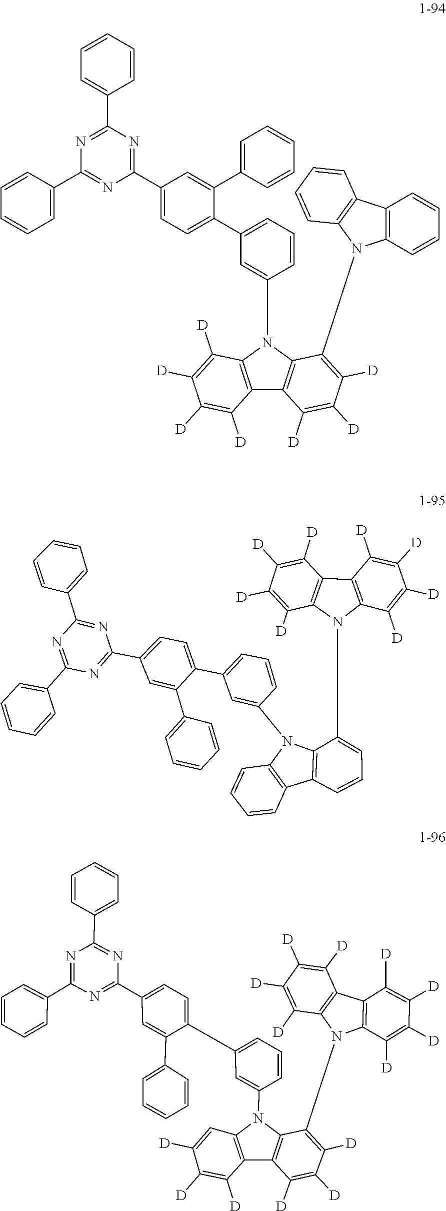 [SMILES: [2H]c1c([2H])c([2H])c2c(c1[2H])c1c([2H])c([2H])c([2H])c(-n3c4ccccc4c4ccccc43)c1n2-c1cccc(-c2ccc(-c3nc(-c4ccccc4)nc(-c4ccccc4)n3)cc2-c2ccccc2)c1 ; [2H]c1c([2H])c([2H])c2c(c1[2H])c1c([2H])c([2H])c([2H])c([2H])c1n2-c1cccc2c3ccccc3n(-c3cccc(-c4ccc(-c5nc(-c6ccccc6)nc(-c6ccccc6)n5)cc4-c4ccccc4)c3)c12 ; [2H]c1cc2c(c([2H])c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccc(-c3nc(-c4ccccc4)nc(-c4ccccc4)n3)cc2-c2ccccc2)c1]

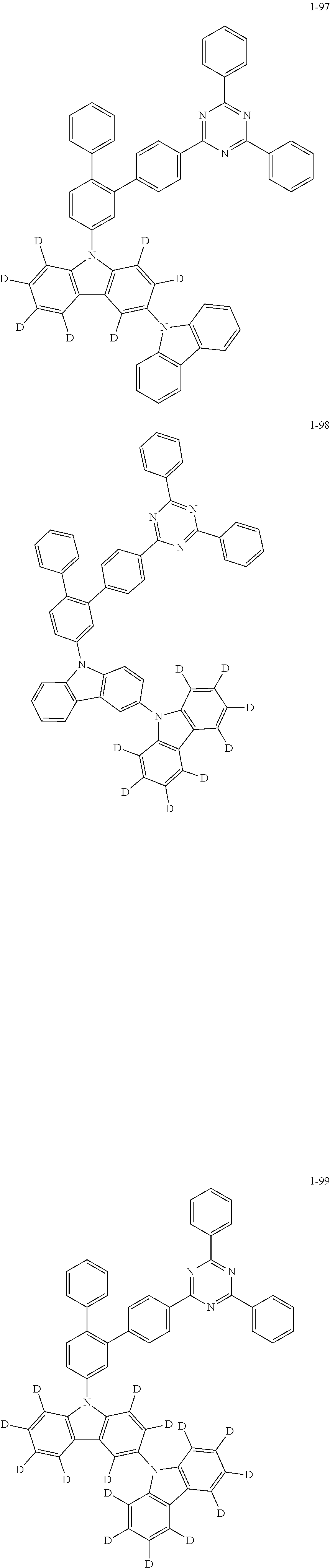

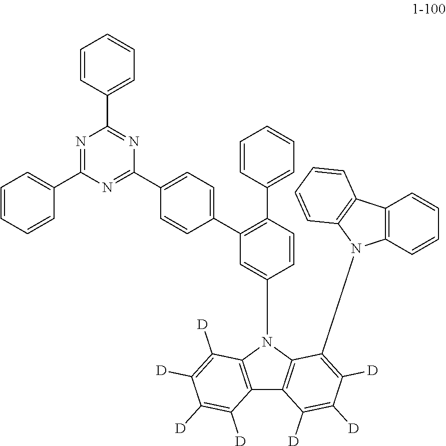 [SMILES: [2H]c1c([2H])c([2H])c2c(c1[2H])c1c([2H])c([2H])c([2H])c(-n3c4ccccc4c4ccccc43)c1n2-c1ccc(-c2ccccc2)c(-c2ccc(-c3nc(-c4ccccc4)nc(-c4ccccc4)n3)cc2)c1]

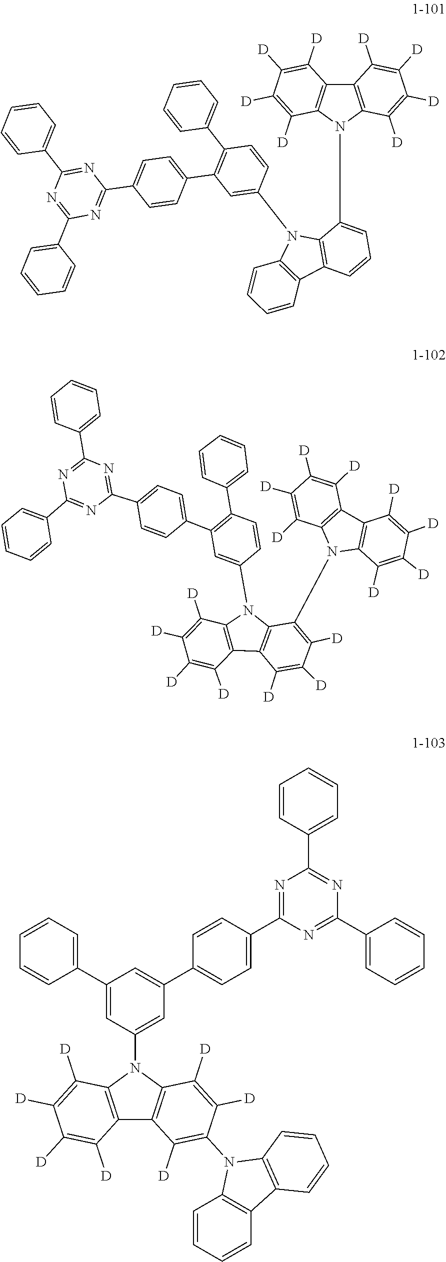 [SMILES: [2H]c1c([2H])c([2H])c2c(c1[2H])c1c([2H])c([2H])c([2H])c([2H])c1n2-c1cccc2c3ccccc3n(-c3ccc(-c4ccccc4)c(-c4ccc(-c5nc(-c6ccccc6)nc(-c6ccccc6)n5)cc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)c(-c2ccc(-c3nc(-c4ccccc4)nc(-c4ccccc4)n3)cc2)c1 ; [2H]c1c([2H])c([2H])c2c(c1[2H])c1c([2H])c(-n3c4ccccc4c4ccccc43)c([2H])c([2H])c1n2-c1cc(-c2ccccc2)cc(-c2ccc(-c3nc(-c4ccccc4)nc(-c4ccccc4)n3)cc2)c1]

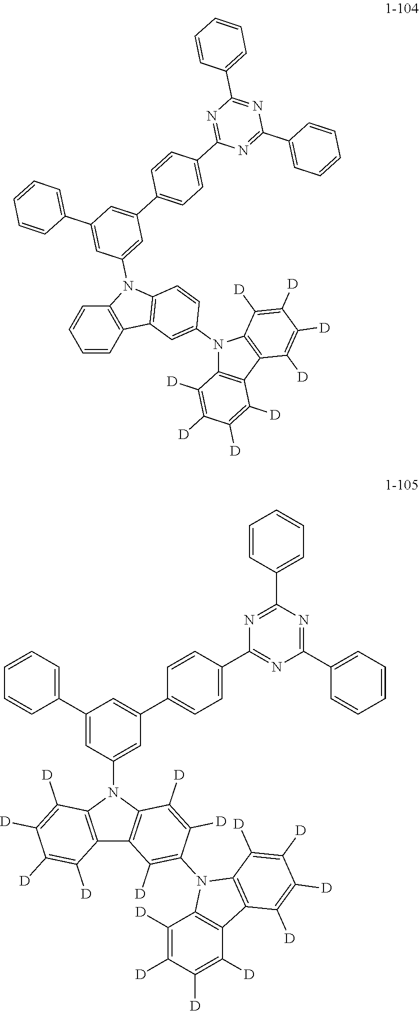 [SMILES: [2H]c1c([2H])c([2H])c2c(c1[2H])c1c([2H])c([2H])c([2H])c([2H])c1n2-c1ccc2c(c1)c1ccccc1n2-c1cc(-c2ccccc2)cc(-c2ccc(-c3nc(-c4ccccc4)nc(-c4ccccc4)n3)cc2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)cc(-c2ccc(-c3nc(-c4ccccc4)nc(-c4ccccc4)n3)cc2)c1]

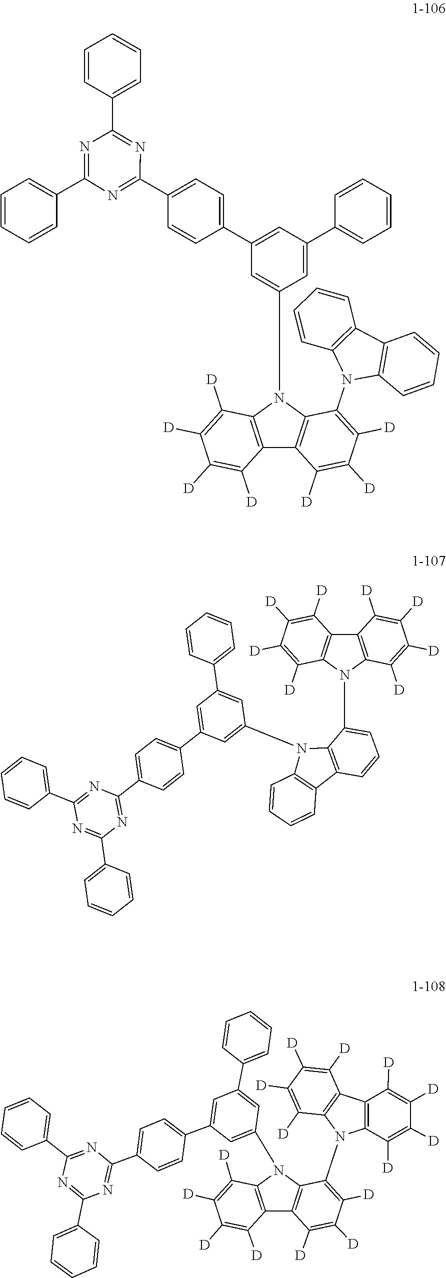

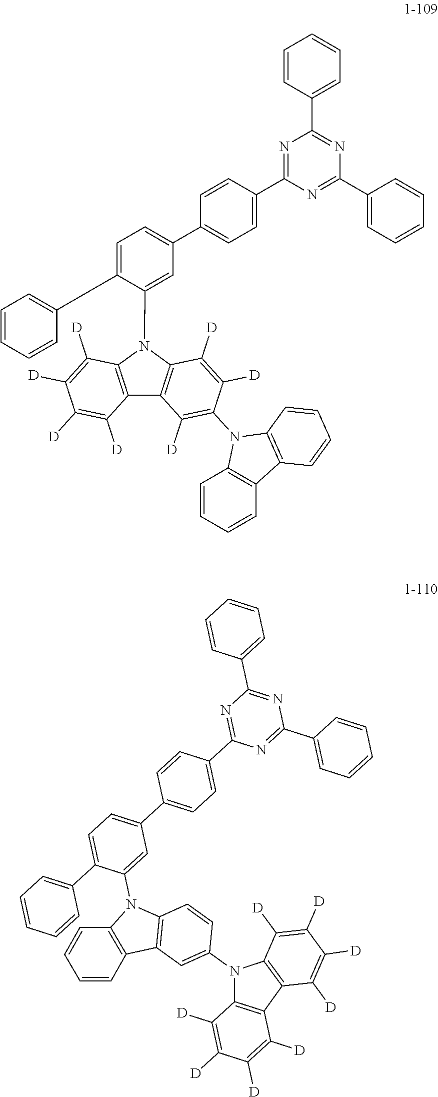 [SMILES: [2H]c1c([2H])c([2H])c2c(c1[2H])c1c([2H])c(-n3c4ccccc4c4ccccc43)c([2H])c([2H])c1n2-c1cc(-c2ccc(-c3nc(-c4ccccc4)nc(-c4ccccc4)n3)cc2)ccc1-c1ccccc1 ; [2H]c1c([2H])c([2H])c2c(c1[2H])c1c([2H])c([2H])c([2H])c([2H])c1n2-c1ccc2c(c1)c1ccccc1n2-c1cc(-c2ccc(-c3nc(-c4ccccc4)nc(-c4ccccc4)n3)cc2)ccc1-c1ccccc1]

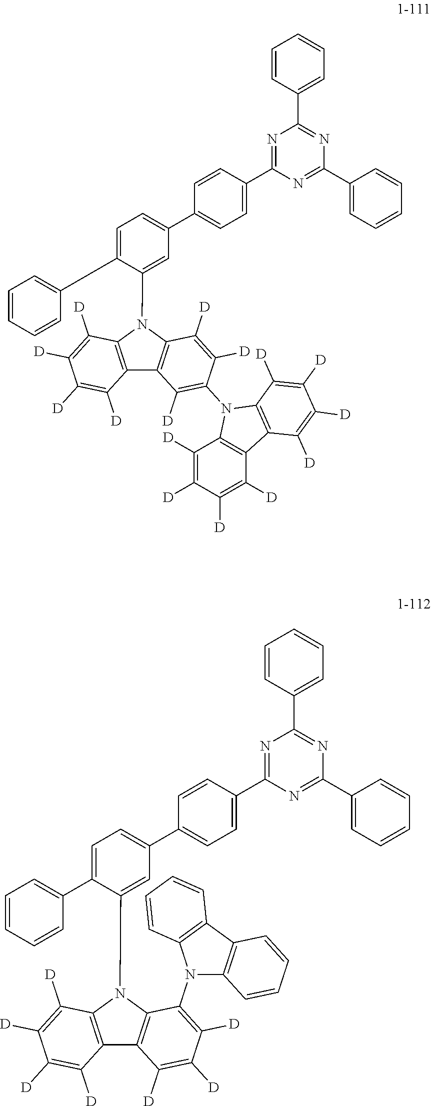 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4ccccc4)n3)cc2)ccc1-c1ccccc1 ; [2H]c1c([2H])c([2H])c2c(c1[2H])c1c([2H])c([2H])c([2H])c(-n3c4ccccc4c4ccccc43)c1n2-c1cc(-c2ccc(-c3nc(-c4ccccc4)nc(-c4ccccc4)n3)cc2)ccc1-c1ccccc1]

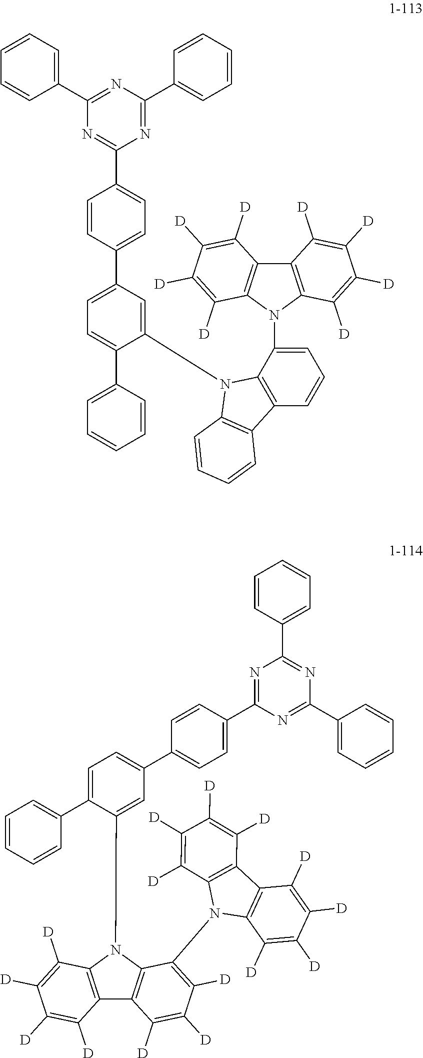 [SMILES: [2H]c1c([2H])c([2H])c2c(c1[2H])c1c([2H])c([2H])c([2H])c([2H])c1n2-c1cccc2c3ccccc3n(-c3cc(-c4ccc(-c5nc(-c6ccccc6)nc(-c6ccccc6)n5)cc4)ccc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccc(-c3nc(-c4ccccc4)nc(-c4ccccc4)n3)cc2)ccc1-c1ccccc1]

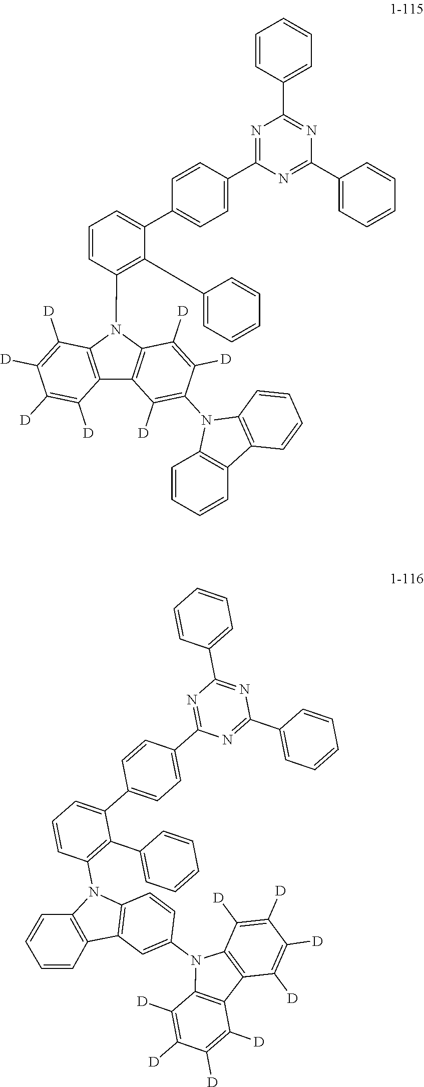 [SMILES: [2H]c1c([2H])c([2H])c2c(c1[2H])c1c([2H])c(-n3c4ccccc4c4ccccc43)c([2H])c([2H])c1n2-c1cccc(-c2ccc(-c3nc(-c4ccccc4)nc(-c4ccccc4)n3)cc2)c1-c1ccccc1 ; [2H]c1c([2H])c([2H])c2c(c1[2H])c1c([2H])c([2H])c([2H])c([2H])c1n2-c1ccc2c(c1)c1ccccc1n2-c1cccc(-c2ccc(-c3nc(-c4ccccc4)nc(-c4ccccc4)n3)cc2)c1-c1ccccc1]

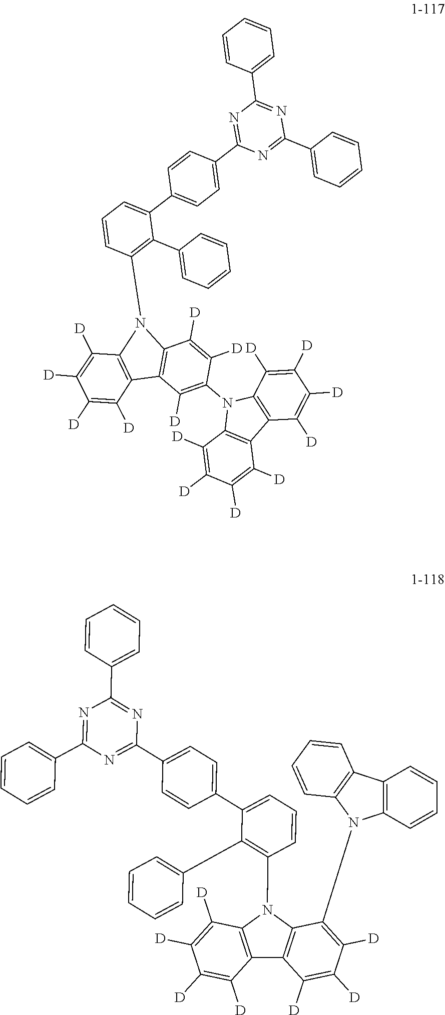 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccc(-c3nc(-c4ccccc4)nc(-c4ccccc4)n3)cc2)c1-c1ccccc1 ; [2H]c1c([2H])c([2H])c2c(c1[2H])c1c([2H])c([2H])c([2H])c(-n3c4ccccc4c4ccccc43)c1n2-c1cccc(-c2ccc(-c3nc(-c4ccccc4)nc(-c4ccccc4)n3)cc2)c1-c1ccccc1]

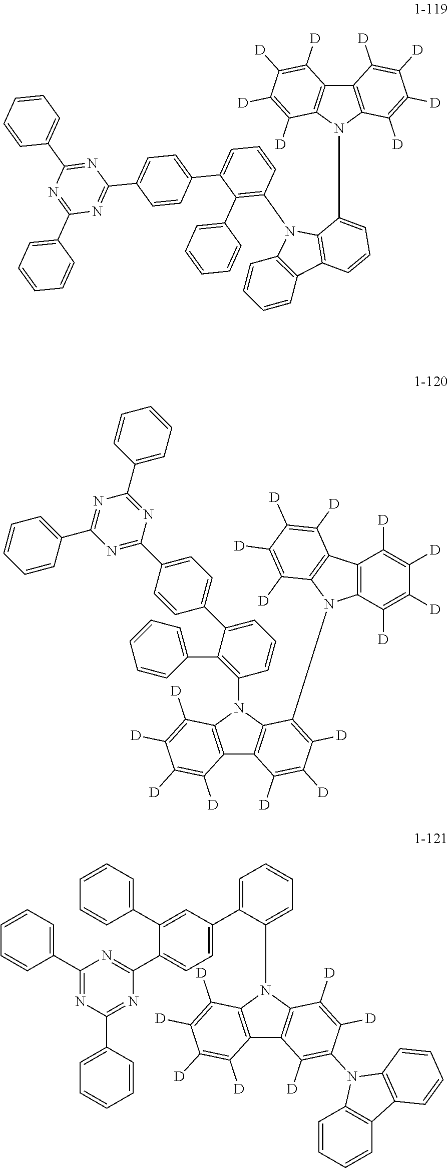 [SMILES: [2H]c1c([2H])c([2H])c2c(c1[2H])c1c([2H])c([2H])c([2H])c([2H])c1n2-c1cccc2c3ccccc3n(-c3cccc(-c4ccc(-c5nc(-c6ccccc6)nc(-c6ccccc6)n5)cc4)c3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccc(-c3nc(-c4ccccc4)nc(-c4ccccc4)n3)cc2)c1-c1ccccc1 ; [2H]c1c([2H])c([2H])c2c(c1[2H])c1c([2H])c(-n3c4ccccc4c4ccccc43)c([2H])c([2H])c1n2-c1ccccc1-c1ccc(-c2nc(-c3ccccc3)nc(-c3ccccc3)n2)c(-c2ccccc2)c1]

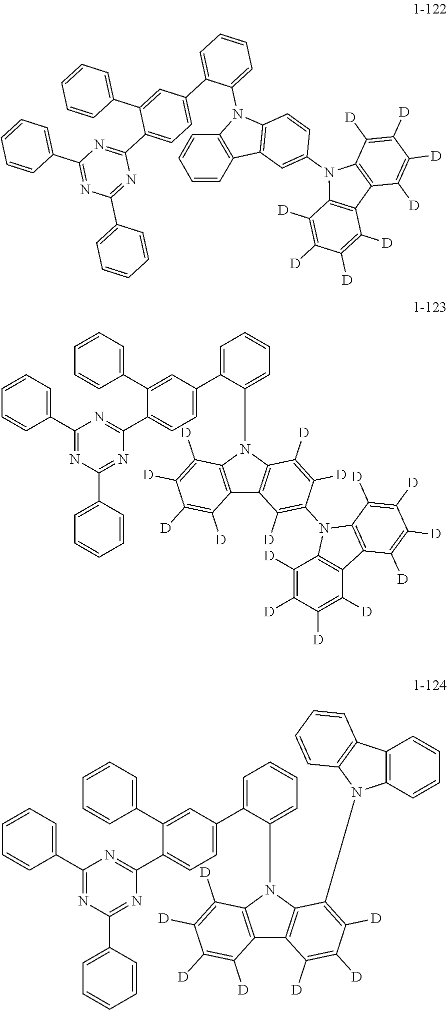 [SMILES: [2H]c1c([2H])c([2H])c2c(c1[2H])c1c([2H])c([2H])c([2H])c([2H])c1n2-c1ccc2c(c1)c1ccccc1n2-c1ccccc1-c1ccc(-c2nc(-c3ccccc3)nc(-c3ccccc3)n2)c(-c2ccccc2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1ccc(-c2nc(-c3ccccc3)nc(-c3ccccc3)n2)c(-c2ccccc2)c1 ; [2H]c1c([2H])c([2H])c2c(c1[2H])c1c([2H])c([2H])c([2H])c(-n3c4ccccc4c4ccccc43)c1n2-c1ccccc1-c1ccc(-c2nc(-c3ccccc3)nc(-c3ccccc3)n2)c(-c2ccccc2)c1]

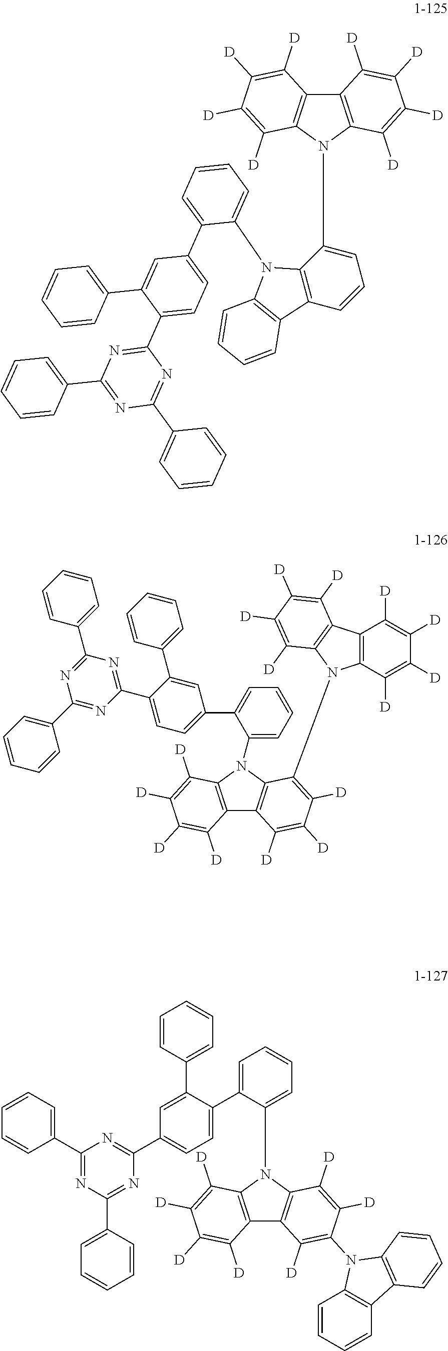 [SMILES: [2H]c1c([2H])c([2H])c2c(c1[2H])c1c([2H])c([2H])c([2H])c([2H])c1n2-c1cccc2c3ccccc3n(-c3ccccc3-c3ccc(-c4nc(-c5ccccc5)nc(-c5ccccc5)n4)c(-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1ccc(-c2nc(-c3ccccc3)nc(-c3ccccc3)n2)c(-c2ccccc2)c1 ; [2H]c1c([2H])c([2H])c2c(c1[2H])c1c([2H])c(-n3c4ccccc4c4ccccc43)c([2H])c([2H])c1n2-c1ccccc1-c1ccc(-c2nc(-c3ccccc3)nc(-c3ccccc3)n2)cc1-c1ccccc1]

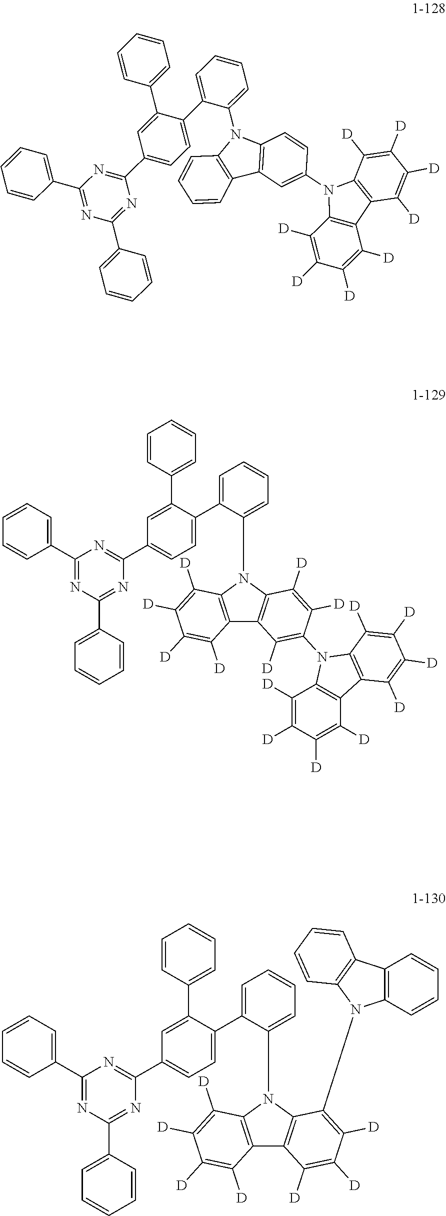 [SMILES: [2H]c1c([2H])c([2H])c2c(c1[2H])c1c([2H])c([2H])c([2H])c([2H])c1n2-c1ccc2c(c1)c1ccccc1n2-c1ccccc1-c1ccc(-c2nc(-c3ccccc3)nc(-c3ccccc3)n2)cc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1ccc(-c2nc(-c3ccccc3)nc(-c3ccccc3)n2)cc1-c1ccccc1 ; [2H]c1c([2H])c([2H])c2c(c1[2H])c1c([2H])c([2H])c([2H])c(-n3c4ccccc4c4ccccc43)c1n2-c1ccccc1-c1ccc(-c2nc(-c3ccccc3)nc(-c3ccccc3)n2)cc1-c1ccccc1]

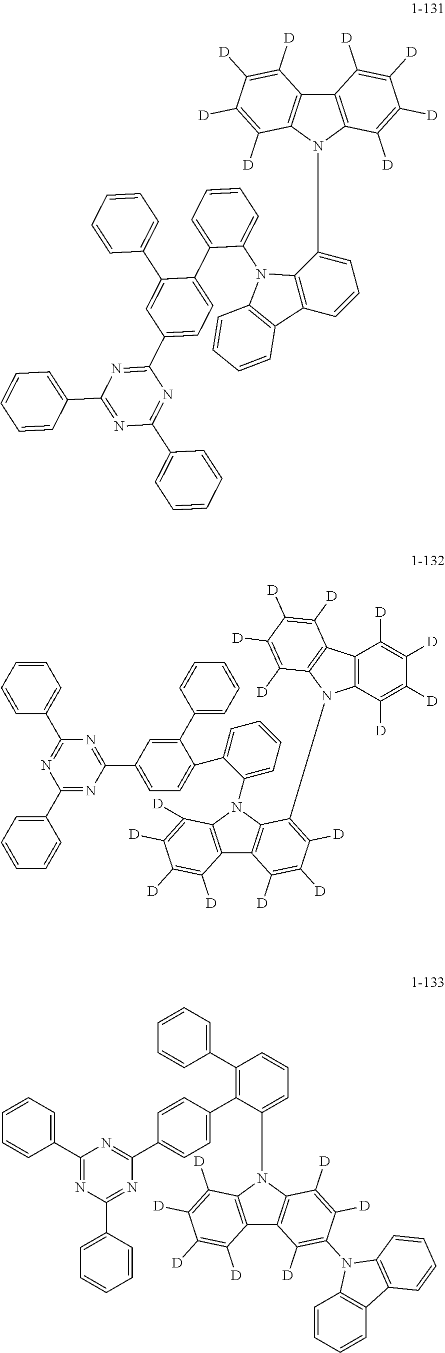 [SMILES: [2H]c1c([2H])c([2H])c2c(c1[2H])c1c([2H])c([2H])c([2H])c([2H])c1n2-c1cccc2c3ccccc3n(-c3ccccc3-c3ccc(-c4nc(-c5ccccc5)nc(-c5ccccc5)n4)cc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1ccc(-c2nc(-c3ccccc3)nc(-c3ccccc3)n2)cc1-c1ccccc1 ; [2H]c1c([2H])c([2H])c2c(c1[2H])c1c([2H])c(-n3c4ccccc4c4ccccc43)c([2H])c([2H])c1n2-c1cccc(-c2ccccc2)c1-c1ccc(-c2nc(-c3ccccc3)nc(-c3ccccc3)n2)cc1]

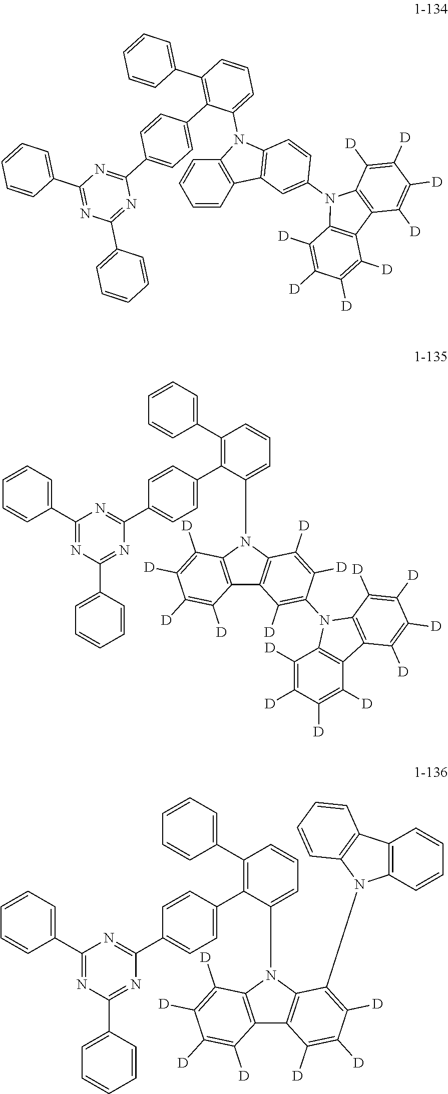 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2ccccc2)c1-c1ccc(-c2nc(-c3ccccc3)nc(-c3ccccc3)n2)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccccc2)c1-c1ccc(-c2nc(-c3ccccc3)nc(-c3ccccc3)n2)cc1 ; [2H]c1c([2H])c([2H])c2c(c1[2H])c1c([2H])c([2H])c([2H])c(-n3c4ccccc4c4ccccc43)c1n2-c1cccc(-c2ccccc2)c1-c1ccc(-c2nc(-c3ccccc3)nc(-c3ccccc3)n2)cc1]

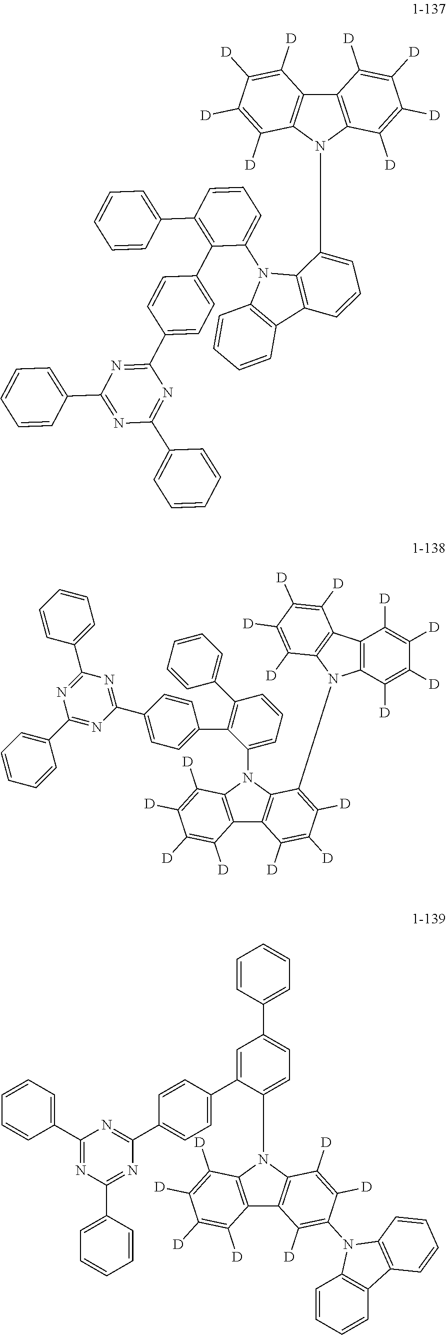 [SMILES: [2H]c1c([2H])c([2H])c2c(c1[2H])c1c([2H])c([2H])c([2H])c([2H])c1n2-c1cccc2c3ccccc3n(-c3cccc(-c4ccccc4)c3-c3ccc(-c4nc(-c5ccccc5)nc(-c5ccccc5)n4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccccc2)c1-c1ccc(-c2nc(-c3ccccc3)nc(-c3ccccc3)n2)cc1 ; [2H]c1c([2H])c([2H])c2c(c1[2H])c1c([2H])c(-n3c4ccccc4c4ccccc43)c([2H])c([2H])c1n2-c1ccc(-c2ccccc2)cc1-c1ccc(-c2nc(-c3ccccc3)nc(-c3ccccc3)n2)cc1]

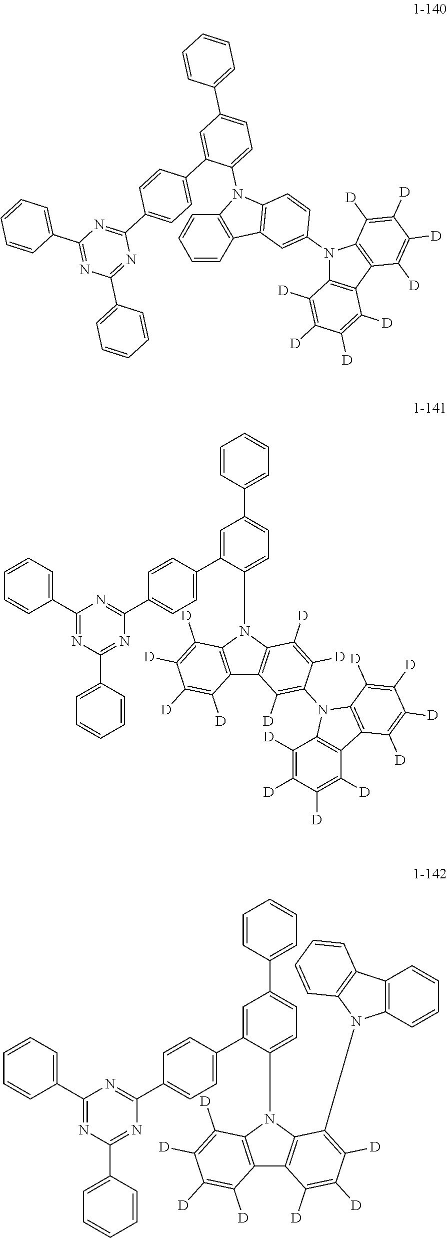 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2ccccc2)cc1-c1ccc(-c2nc(-c3ccccc3)nc(-c3ccccc3)n2)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)cc1-c1ccc(-c2nc(-c3ccccc3)nc(-c3ccccc3)n2)cc1 ; [2H]c1c([2H])c([2H])c2c(c1[2H])c1c([2H])c([2H])c([2H])c(-n3c4ccccc4c4ccccc43)c1n2-c1ccc(-c2ccccc2)cc1-c1ccc(-c2nc(-c3ccccc3)nc(-c3ccccc3)n2)cc1]

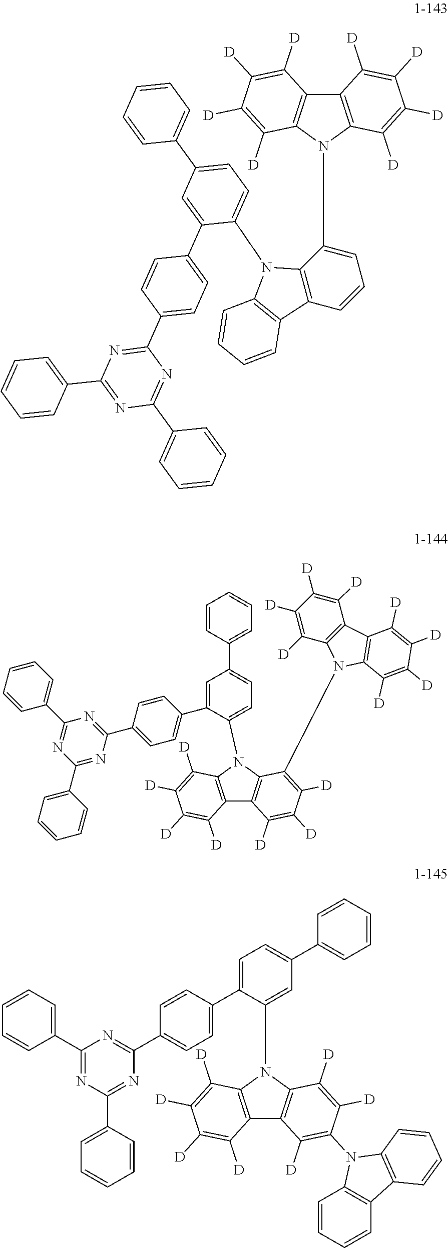 [SMILES: [2H]c1c([2H])c([2H])c2c(c1[2H])c1c([2H])c([2H])c([2H])c([2H])c1n2-c1cccc2c3ccccc3n(-c3ccc(-c4ccccc4)cc3-c3ccc(-c4nc(-c5ccccc5)nc(-c5ccccc5)n4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)cc1-c1ccc(-c2nc(-c3ccccc3)nc(-c3ccccc3)n2)cc1 ; [2H]c1c([2H])c([2H])c2c(c1[2H])c1c([2H])c(-n3c4ccccc4c4ccccc43)c([2H])c([2H])c1n2-c1cc(-c2ccccc2)ccc1-c1ccc(-c2nc(-c3ccccc3)nc(-c3ccccc3)n2)cc1]

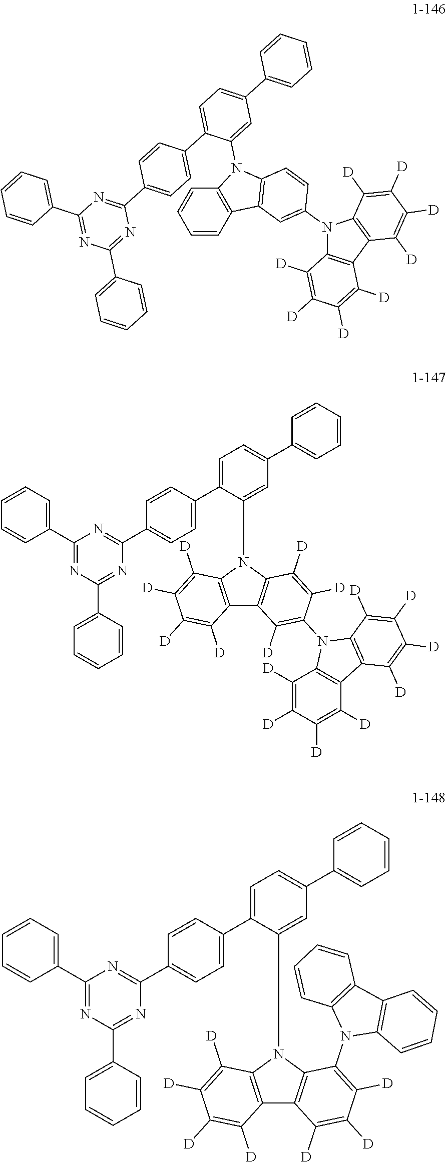 [SMILES: [2H]c1c([2H])c([2H])c2c(c1[2H])c1c([2H])c([2H])c([2H])c([2H])c1n2-c1ccc2c(c1)c1ccccc1n2-c1cc(-c2ccccc2)ccc1-c1ccc(-c2nc(-c3ccccc3)nc(-c3ccccc3)n2)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)ccc1-c1ccc(-c2nc(-c3ccccc3)nc(-c3ccccc3)n2)cc1 ; [2H]c1c([2H])c([2H])c2c(c1[2H])c1c([2H])c([2H])c([2H])c(-n3c4ccccc4c4ccccc43)c1n2-c1cc(-c2ccccc2)ccc1-c1ccc(-c2nc(-c3ccccc3)nc(-c3ccccc3)n2)cc1]

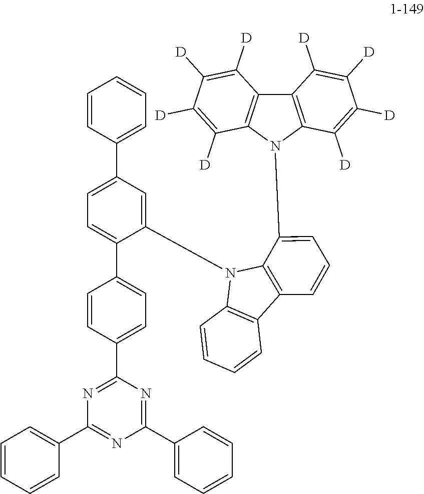 [SMILES: [2H]c1c([2H])c([2H])c2c(c1[2H])c1c([2H])c([2H])c([2H])c([2H])c1n2-c1cccc2c3ccccc3n(-c3cc(-c4ccccc4)ccc3-c3ccc(-c4nc(-c5ccccc5)nc(-c5ccccc5)n4)cc3)c12]

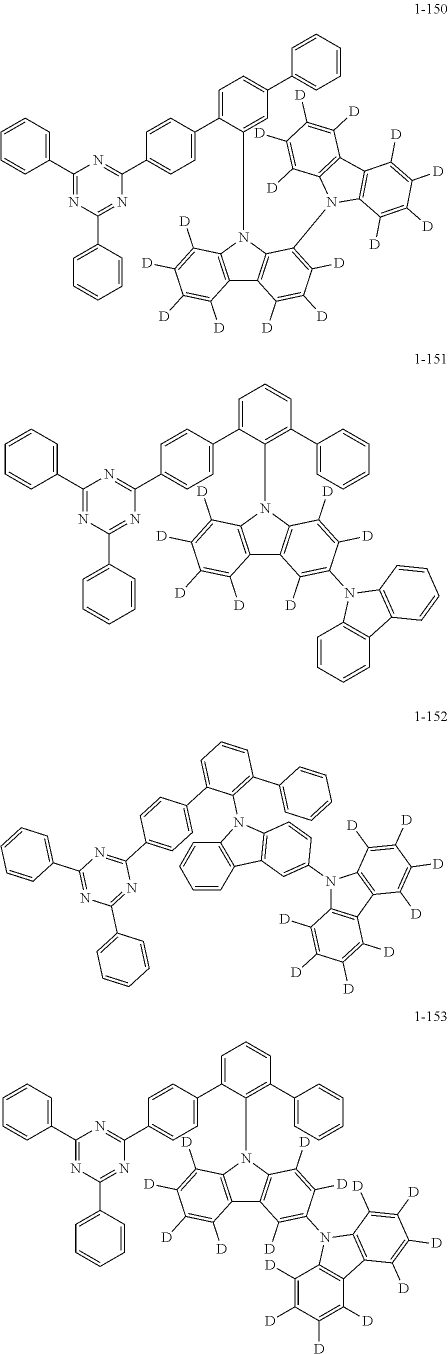

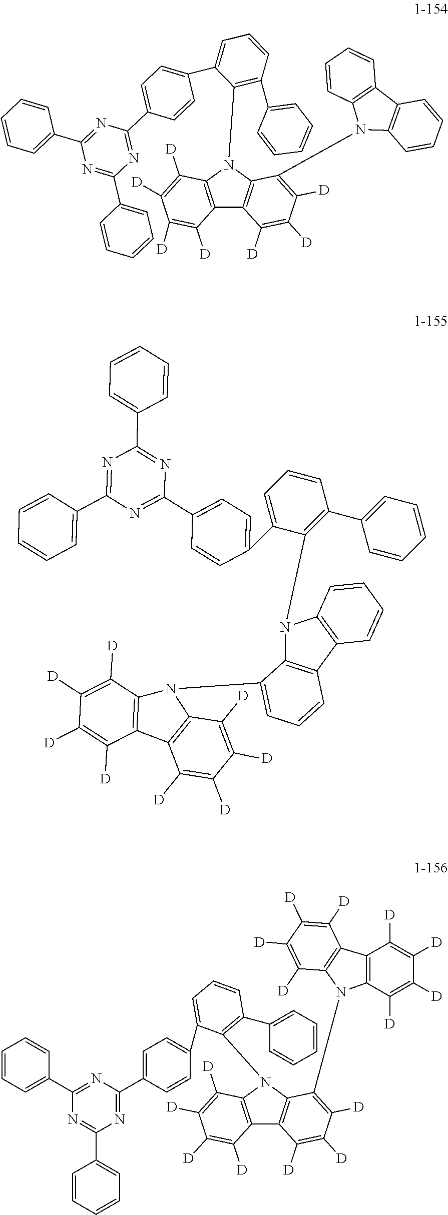 [SMILES: [2H]c1c([2H])c([2H])c2c(c1[2H])c1c([2H])c([2H])c([2H])c(-n3c4ccccc4c4ccccc43)c1n2-c1c(-c2ccccc2)cccc1-c1ccc(-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1cccc2c3ccccc3n(-c3c(-c4ccccc4)cccc3-c3ccc(-c4nc(-c5ccccc5)nc(-c5ccccc5)n4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1c(-c2ccccc2)cccc1-c1ccc(-c2nc(-c3ccccc3)nc(-c3ccccc3)n2)cc1]

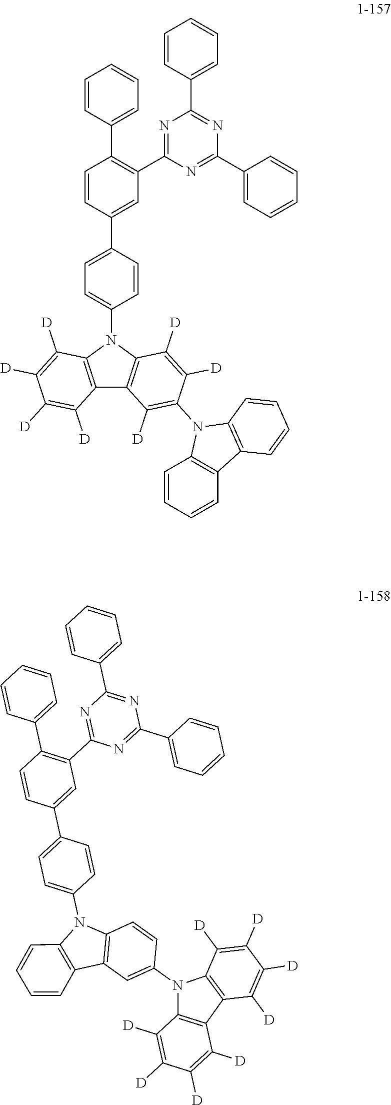 [SMILES: [2H]c1c([2H])c([2H])c2c(c1[2H])c1c([2H])c(-n3c4ccccc4c4ccccc43)c([2H])c([2H])c1n2-c1ccc(-c2ccc(-c3ccccc3)c(-c3nc(-c4ccccc4)nc(-c4ccccc4)n3)c2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3ccccc3)c(-c3nc(-c4ccccc4)nc(-c4ccccc4)n3)c2)cc1]

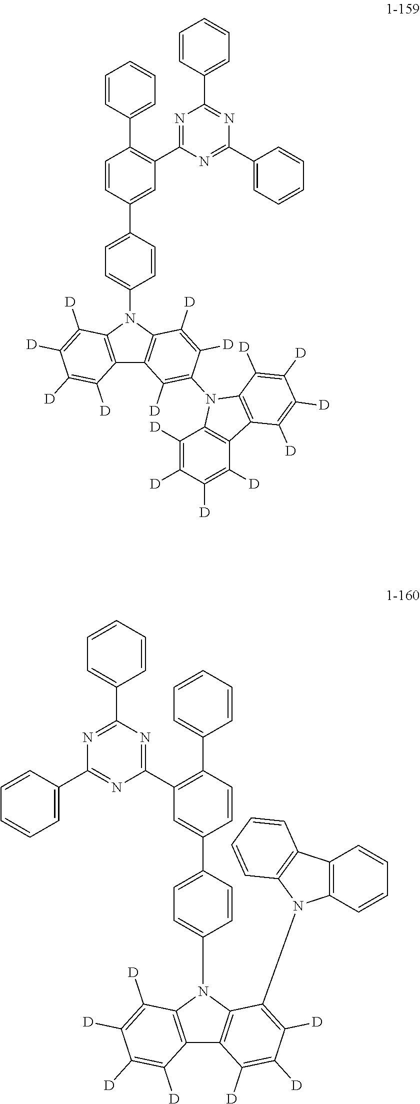 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3ccccc3)c(-c3nc(-c4ccccc4)nc(-c4ccccc4)n3)c2)cc1 ; [2H]c1c([2H])c([2H])c2c(c1[2H])c1c([2H])c([2H])c([2H])c(-n3c4ccccc4c4ccccc43)c1n2-c1ccc(-c2ccc(-c3ccccc3)c(-c3nc(-c4ccccc4)nc(-c4ccccc4)n3)c2)cc1]

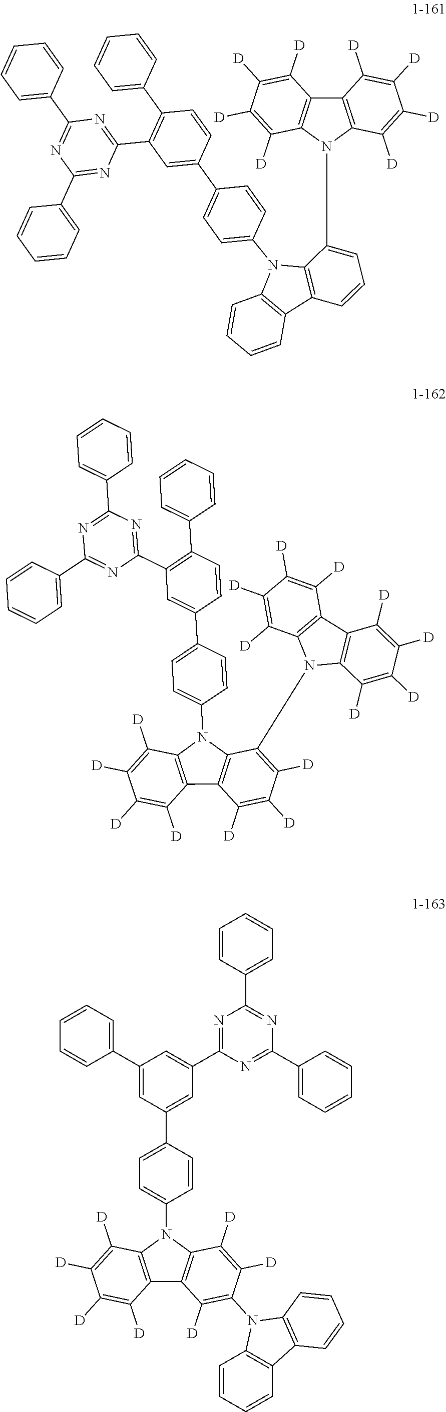 [SMILES: [2H]c1c([2H])c([2H])c2c(c1[2H])c1c([2H])c([2H])c([2H])c([2H])c1n2-c1cccc2c3ccccc3n(-c3ccc(-c4ccc(-c5ccccc5)c(-c5nc(-c6ccccc6)nc(-c6ccccc6)n5)c4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3ccccc3)c(-c3nc(-c4ccccc4)nc(-c4ccccc4)n3)c2)cc1 ; [2H]c1c([2H])c([2H])c2c(c1[2H])c1c([2H])c(-n3c4ccccc4c4ccccc43)c([2H])c([2H])c1n2-c1ccc(-c2cc(-c3ccccc3)cc(-c3nc(-c4ccccc4)nc(-c4ccccc4)n3)c2)cc1]

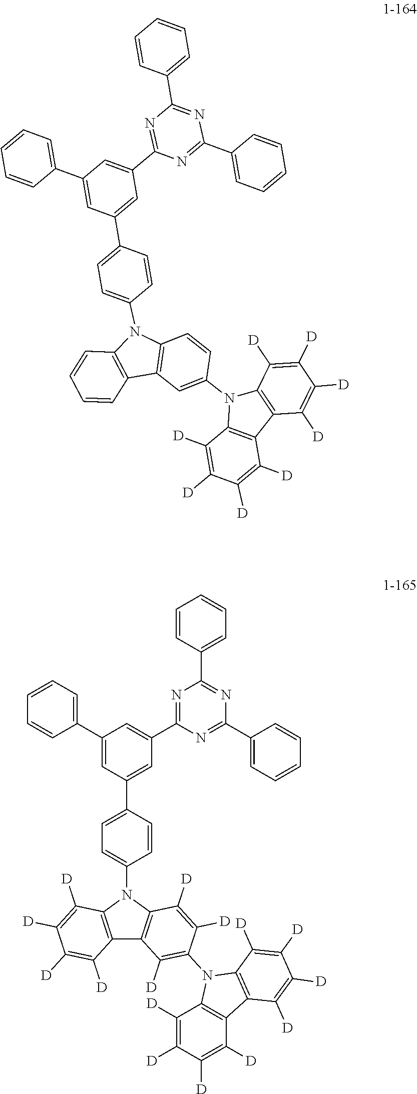 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2cc(-c3ccccc3)cc(-c3nc(-c4ccccc4)nc(-c4ccccc4)n3)c2)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cc(-c3ccccc3)cc(-c3nc(-c4ccccc4)nc(-c4ccccc4)n3)c2)cc1]

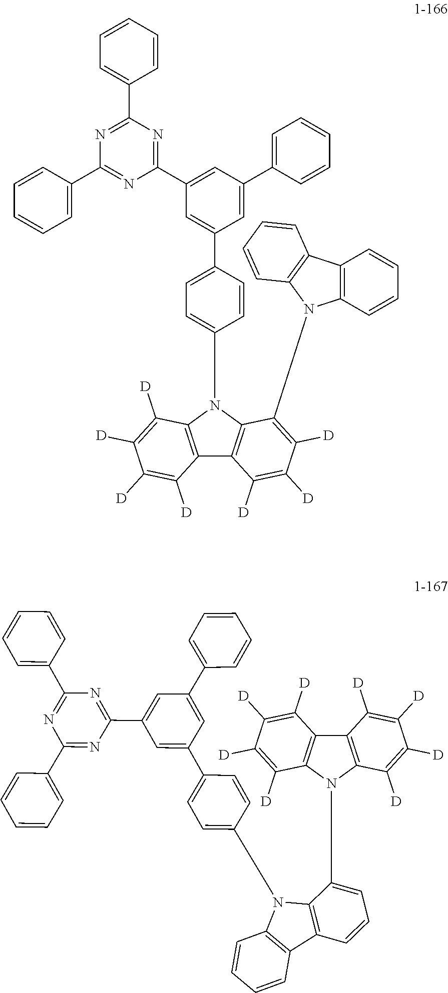 [SMILES: [2H]c1c([2H])c([2H])c2c(c1[2H])c1c([2H])c([2H])c([2H])c(-n3c4ccccc4c4ccccc43)c1n2-c1ccc(-c2cc(-c3ccccc3)cc(-c3nc(-c4ccccc4)nc(-c4ccccc4)n3)c2)cc1 ; [2H]c1c([2H])c([2H])c2c(c1[2H])c1c([2H])c([2H])c([2H])c([2H])c1n2-c1cccc2c3ccccc3n(-c3ccc(-c4cc(-c5ccccc5)cc(-c5nc(-c6ccccc6)nc(-c6ccccc6)n5)c4)cc3)c12]

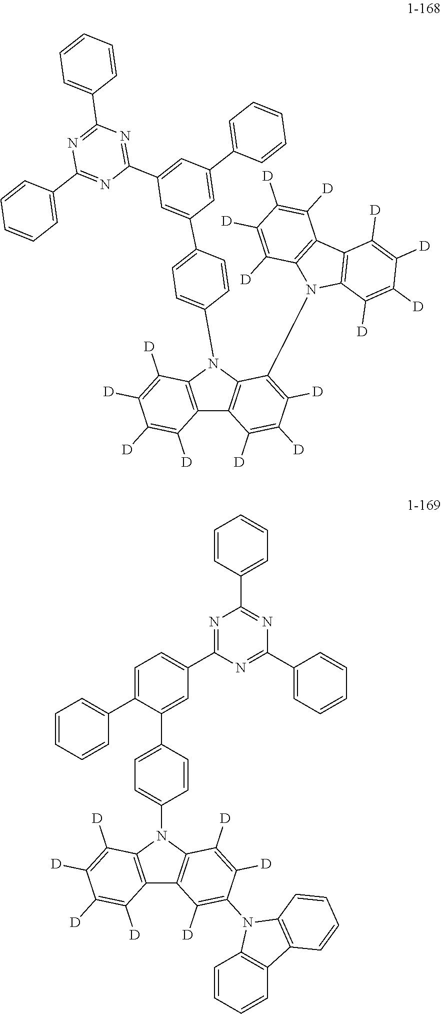 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2cc(-c3ccccc3)cc(-c3nc(-c4ccccc4)nc(-c4ccccc4)n3)c2)cc1 ; [2H]c1c([2H])c([2H])c2c(c1[2H])c1c([2H])c(-n3c4ccccc4c4ccccc43)c([2H])c([2H])c1n2-c1ccc(-c2cc(-c3nc(-c4ccccc4)nc(-c4ccccc4)n3)ccc2-c2ccccc2)cc1]

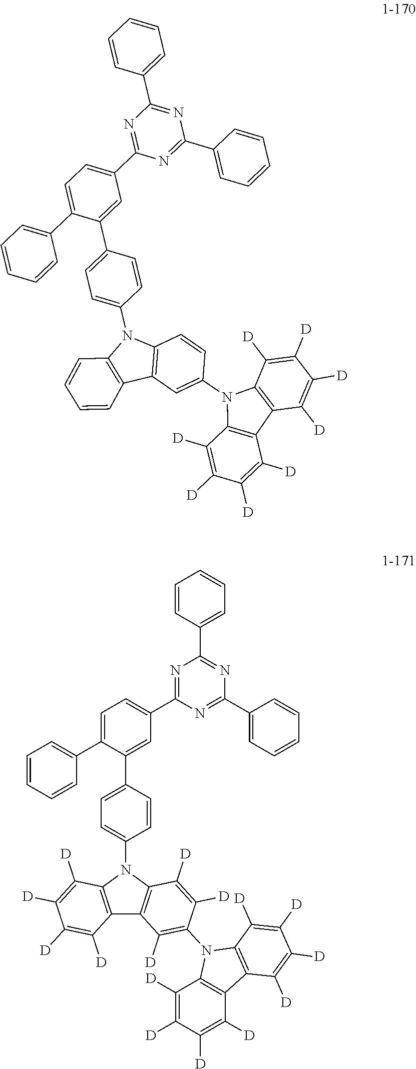 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2cc(-c3nc(-c4ccccc4)nc(-c4ccccc4)n3)ccc2-c2ccccc2)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cc(-c3nc(-c4ccccc4)nc(-c4ccccc4)n3)ccc2-c2ccccc2)cc1]

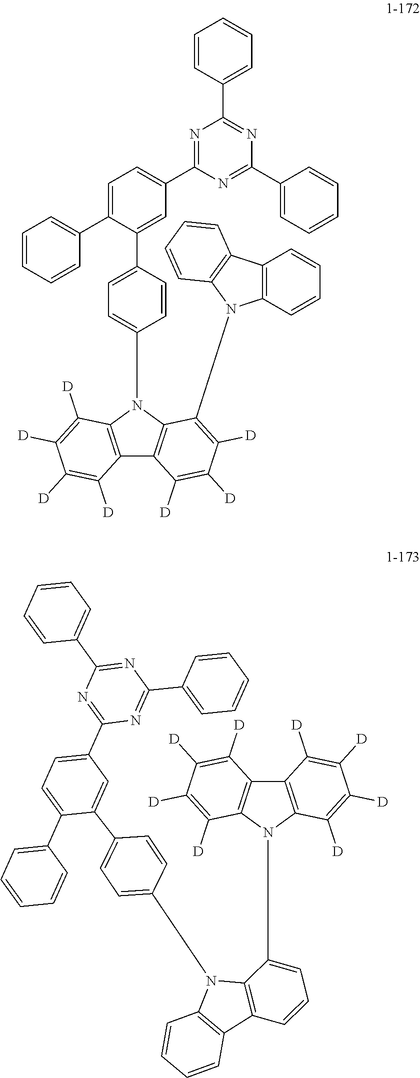 [SMILES: [2H]c1c([2H])c([2H])c2c(c1[2H])c1c([2H])c([2H])c([2H])c(-n3c4ccccc4c4ccccc43)c1n2-c1ccc(-c2cc(-c3nc(-c4ccccc4)nc(-c4ccccc4)n3)ccc2-c2ccccc2)cc1 ; [2H]c1c([2H])c([2H])c2c(c1[2H])c1c([2H])c([2H])c([2H])c([2H])c1n2-c1cccc2c3ccccc3n(-c3ccc(-c4cc(-c5nc(-c6ccccc6)nc(-c6ccccc6)n5)ccc4-c4ccccc4)cc3)c12]

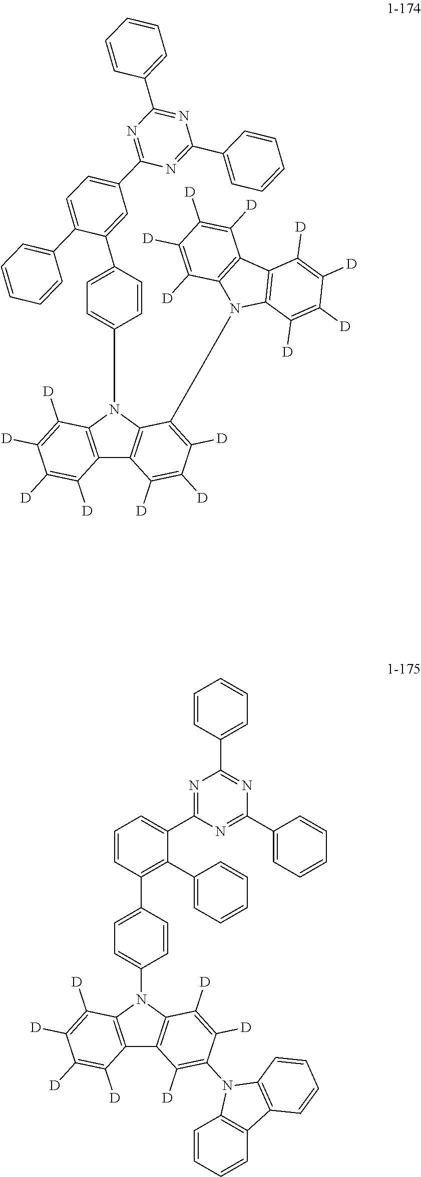 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2cc(-c3nc(-c4ccccc4)nc(-c4ccccc4)n3)ccc2-c2ccccc2)cc1 ; [2H]c1c([2H])c([2H])c2c(c1[2H])c1c([2H])c(-n3c4ccccc4c4ccccc43)c([2H])c([2H])c1n2-c1ccc(-c2cccc(-c3nc(-c4ccccc4)nc(-c4ccccc4)n3)c2-c2ccccc2)cc1]

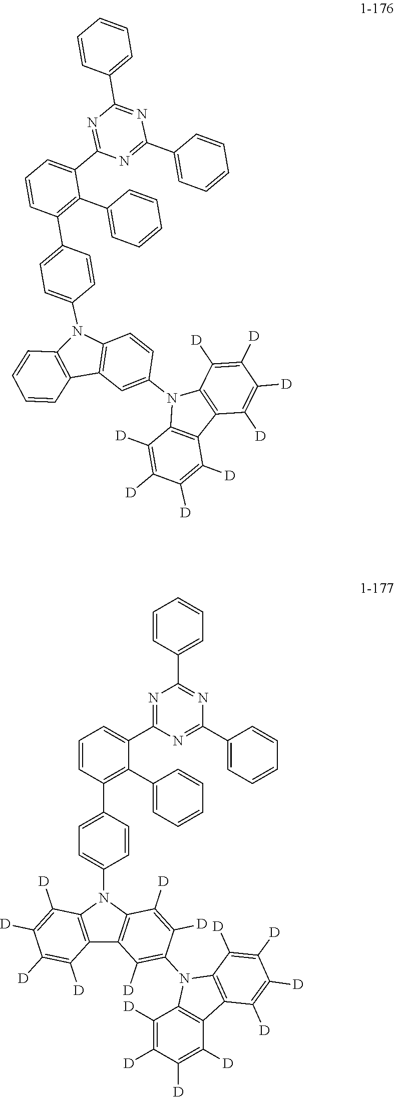 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2cccc(-c3nc(-c4ccccc4)nc(-c4ccccc4)n3)c2-c2ccccc2)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cccc(-c3nc(-c4ccccc4)nc(-c4ccccc4)n3)c2-c2ccccc2)cc1]

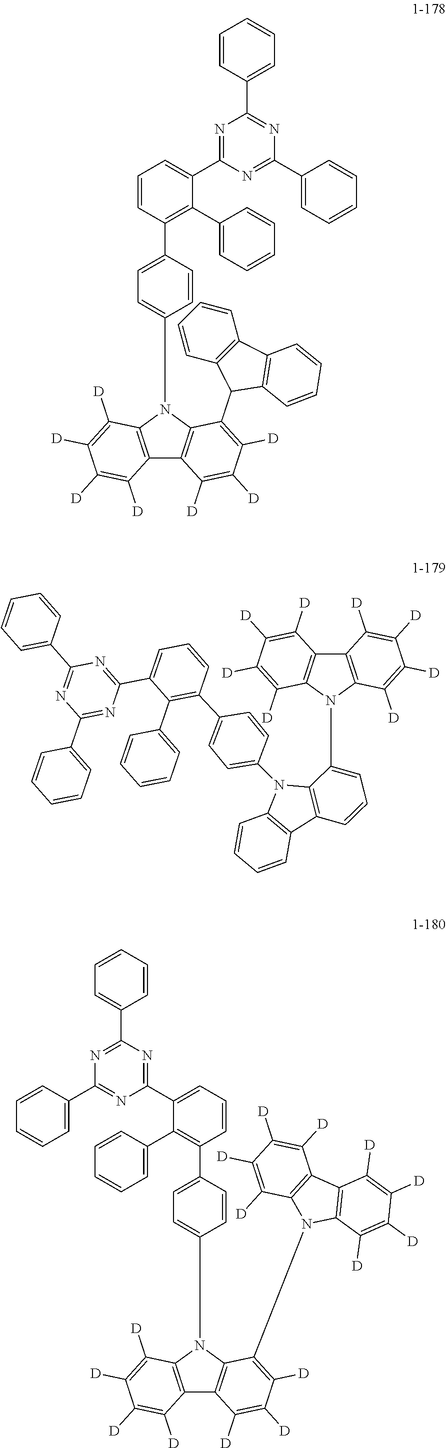 [SMILES: [2H]c1c([2H])c([2H])c2c(c1[2H])c1c([2H])c([2H])c([2H])c(C3c4ccccc4-c4ccccc43)c1n2-c1ccc(-c2cccc(-c3nc(-c4ccccc4)nc(-c4ccccc4)n3)c2-c2ccccc2)cc1 ; [2H]c1c([2H])c([2H])c2c(c1[2H])c1c([2H])c([2H])c([2H])c([2H])c1n2-c1cccc2c3ccccc3n(-c3ccc(-c4cccc(-c5nc(-c6ccccc6)nc(-c6ccccc6)n5)c4-c4ccccc4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2cccc(-c3nc(-c4ccccc4)nc(-c4ccccc4)n3)c2-c2ccccc2)cc1]

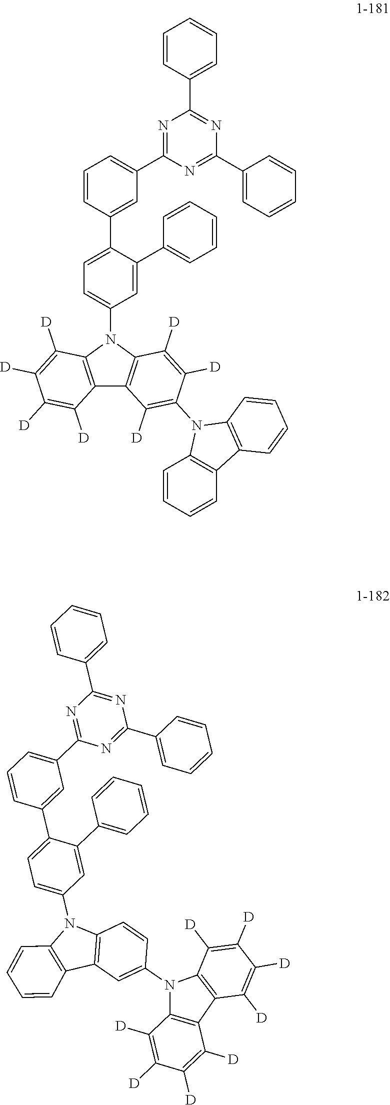 [SMILES: [2H]c1c([2H])c([2H])c2c(c1[2H])c1c([2H])c(-n3c4ccccc4c4ccccc43)c([2H])c([2H])c1n2-c1ccc(-c2cccc(-c3nc(-c4ccccc4)nc(-c4ccccc4)n3)c2)c(-c2ccccc2)c1 ; [2H]c1c([2H])c([2H])c2c(c1[2H])c1c([2H])c([2H])c([2H])c([2H])c1n2-c1ccc2c(c1)c1ccccc1n2-c1ccc(-c2cccc(-c3nc(-c4ccccc4)nc(-c4ccccc4)n3)c2)c(-c2ccccc2)c1]

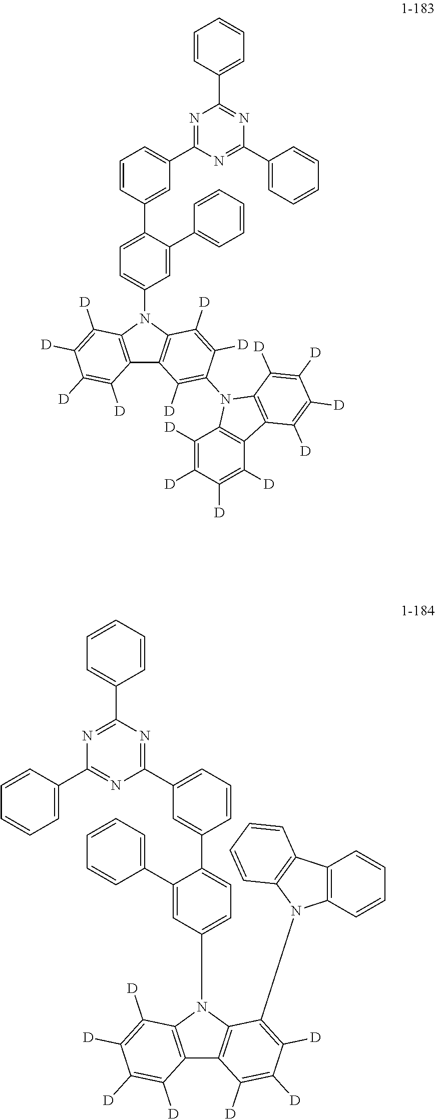 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cccc(-c3nc(-c4ccccc4)nc(-c4ccccc4)n3)c2)c(-c2ccccc2)c1 ; [2H]c1c([2H])c([2H])c2c(c1[2H])c1c([2H])c([2H])c([2H])c(-n3c4ccccc4c4ccccc43)c1n2-c1ccc(-c2cccc(-c3nc(-c4ccccc4)nc(-c4ccccc4)n3)c2)c(-c2ccccc2)c1]

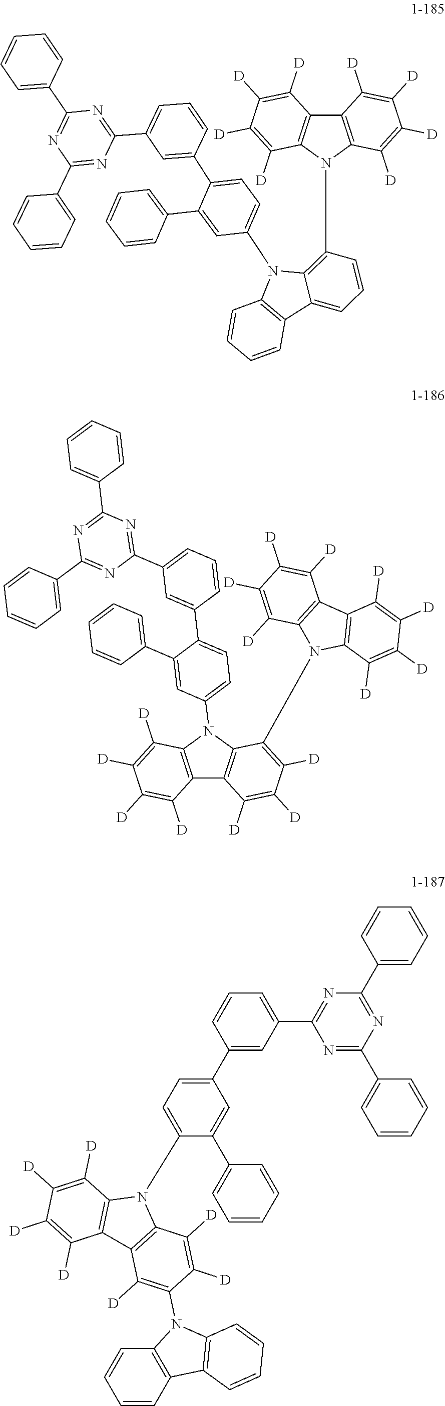 [SMILES: [2H]c1c([2H])c([2H])c2c(c1[2H])c1c([2H])c([2H])c([2H])c([2H])c1n2-c1cccc2c3ccccc3n(-c3ccc(-c4cccc(-c5nc(-c6ccccc6)nc(-c6ccccc6)n5)c4)c(-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2cccc(-c3nc(-c4ccccc4)nc(-c4ccccc4)n3)c2)c(-c2ccccc2)c1 ; [2H]c1c([2H])c([2H])c2c(c1[2H])c1c([2H])c(-n3c4ccccc4c4ccccc43)c([2H])c([2H])c1n2-c1ccc(-c2cccc(-c3nc(-c4ccccc4)nc(-c4ccccc4)n3)c2)cc1-c1ccccc1]

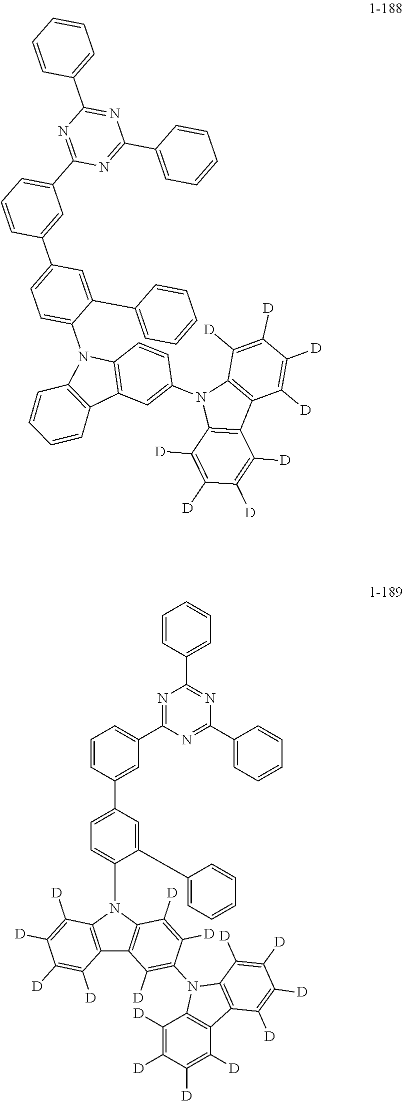 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2cccc(-c3nc(-c4ccccc4)nc(-c4ccccc4)n3)c2)cc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cccc(-c3nc(-c4ccccc4)nc(-c4ccccc4)n3)c2)cc1-c1ccccc1]

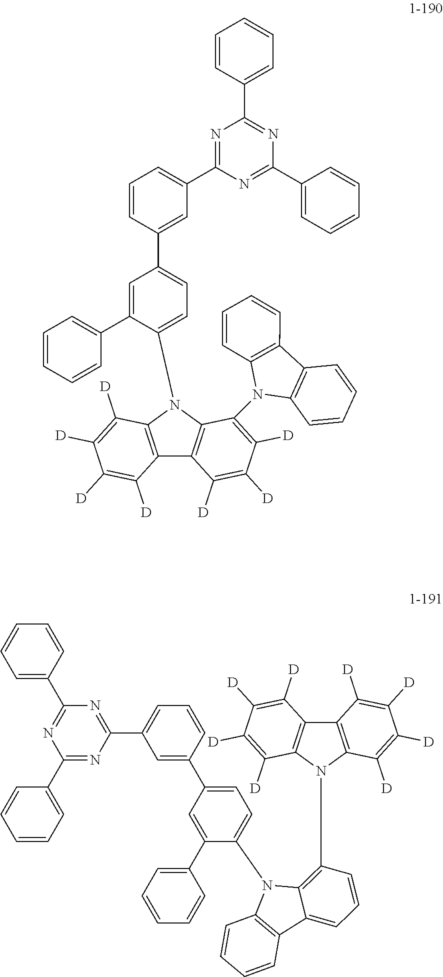 [SMILES: [2H]c1c([2H])c([2H])c2c(c1[2H])c1c([2H])c([2H])c([2H])c(-n3c4ccccc4c4ccccc43)c1n2-c1ccc(-c2cccc(-c3nc(-c4ccccc4)nc(-c4ccccc4)n3)c2)cc1-c1ccccc1 ; [2H]c1c([2H])c([2H])c2c(c1[2H])c1c([2H])c([2H])c([2H])c([2H])c1n2-c1cccc2c3ccccc3n(-c3ccc(-c4cccc(-c5nc(-c6ccccc6)nc(-c6ccccc6)n5)c4)cc3-c3ccccc3)c12]

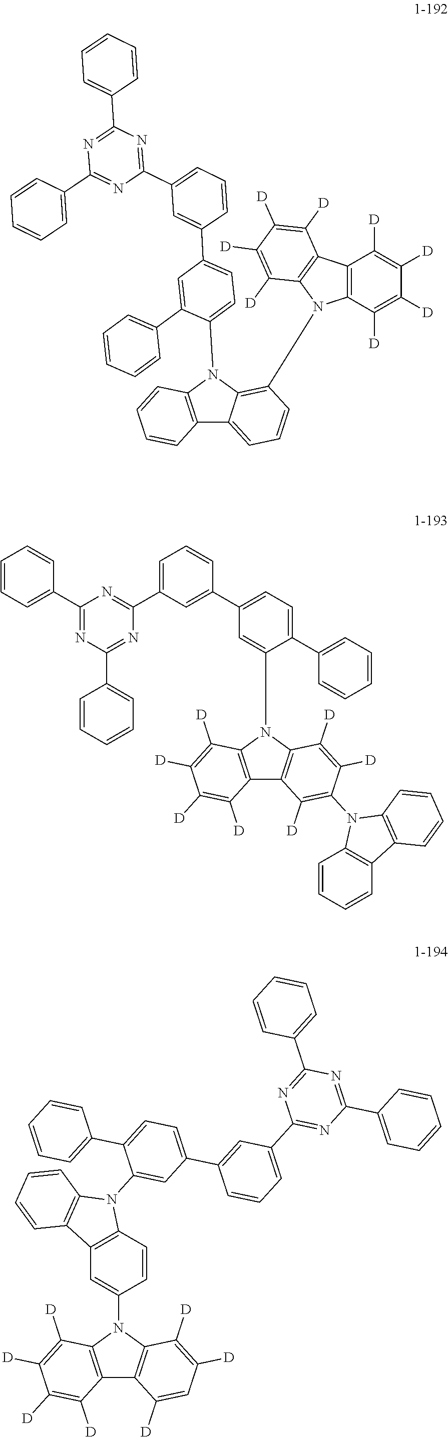 [SMILES: [2H]c1c([2H])c([2H])c2c(c1[2H])c1c([2H])c([2H])c([2H])c([2H])c1n2-c1cccc2c3ccccc3n(-c3ccc(-c4cccc(-c5nc(-c6ccccc6)nc(-c6ccccc6)n5)c4)cc3-c3ccccc3)c12 ; [2H]c1c([2H])c([2H])c2c(c1[2H])c1c([2H])c(-n3c4ccccc4c4ccccc43)c([2H])c([2H])c1n2-c1cc(-c2cccc(-c3nc(-c4ccccc4)nc(-c4ccccc4)n3)c2)ccc1-c1ccccc1 ; [2H]c1cc([2H])c2c3c([2H])c([2H])c([2H])c([2H])c3n(-c3ccc4c(c3)c3ccccc3n4-c3cc(-c4cccc(-c5nc(-c6ccccc6)nc(-c6ccccc6)n5)c4)ccc3-c3ccccc3)c2c1[2H]]

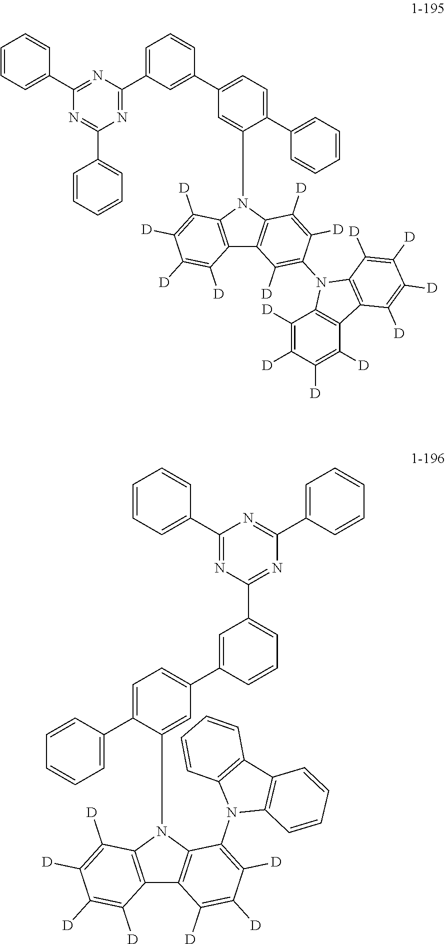 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2cccc(-c3nc(-c4ccccc4)nc(-c4ccccc4)n3)c2)ccc1-c1ccccc1 ; [2H]c1c([2H])c([2H])c2c(c1[2H])c1c([2H])c([2H])c([2H])c(-n3c4ccccc4c4ccccc43)c1n2-c1cc(-c2cccc(-c3nc(-c4ccccc4)nc(-c4ccccc4)n3)c2)ccc1-c1ccccc1]

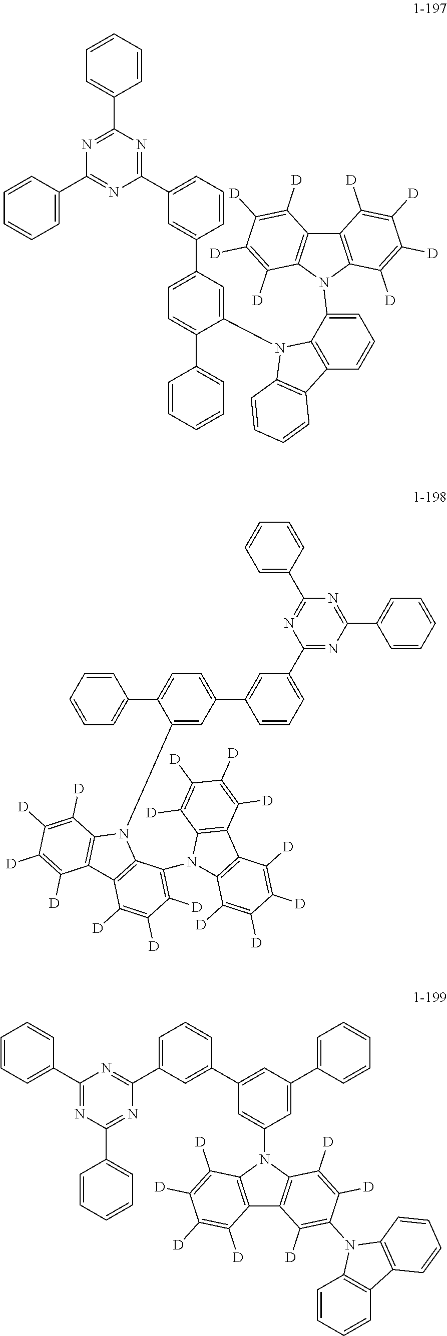 [SMILES: [2H]c1c([2H])c([2H])c2c(c1[2H])c1c([2H])c([2H])c([2H])c([2H])c1n2-c1cccc2c3ccccc3n(-c3cc(-c4cccc(-c5nc(-c6ccccc6)nc(-c6ccccc6)n5)c4)ccc3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2cccc(-c3nc(-c4ccccc4)nc(-c4ccccc4)n3)c2)ccc1-c1ccccc1 ; [2H]c1c([2H])c([2H])c2c(c1[2H])c1c([2H])c(-n3c4ccccc4c4ccccc43)c([2H])c([2H])c1n2-c1cc(-c2ccccc2)cc(-c2cccc(-c3nc(-c4ccccc4)nc(-c4ccccc4)n3)c2)c1]

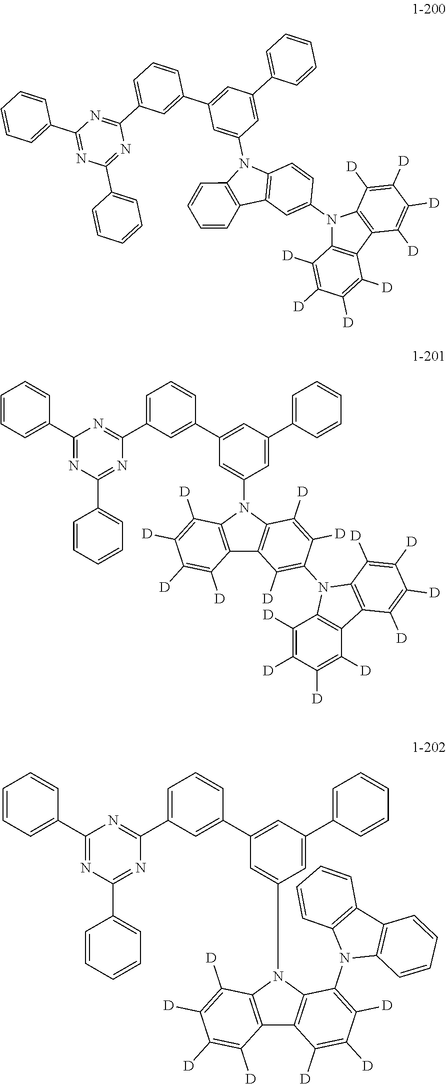 [SMILES: [2H]c1c([2H])c([2H])c2c(c1[2H])c1c([2H])c([2H])c([2H])c([2H])c1n2-c1ccc2c(c1)c1ccccc1n2-c1cc(-c2ccccc2)cc(-c2cccc(-c3nc(-c4ccccc4)nc(-c4ccccc4)n3)c2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)cc(-c2cccc(-c3nc(-c4ccccc4)nc(-c4ccccc4)n3)c2)c1 ; [2H]c1c([2H])c([2H])c2c(c1[2H])c1c([2H])c([2H])c([2H])c(-n3c4ccccc4c4ccccc43)c1n2-c1cc(-c2ccccc2)cc(-c2cccc(-c3nc(-c4ccccc4)nc(-c4ccccc4)n3)c2)c1]

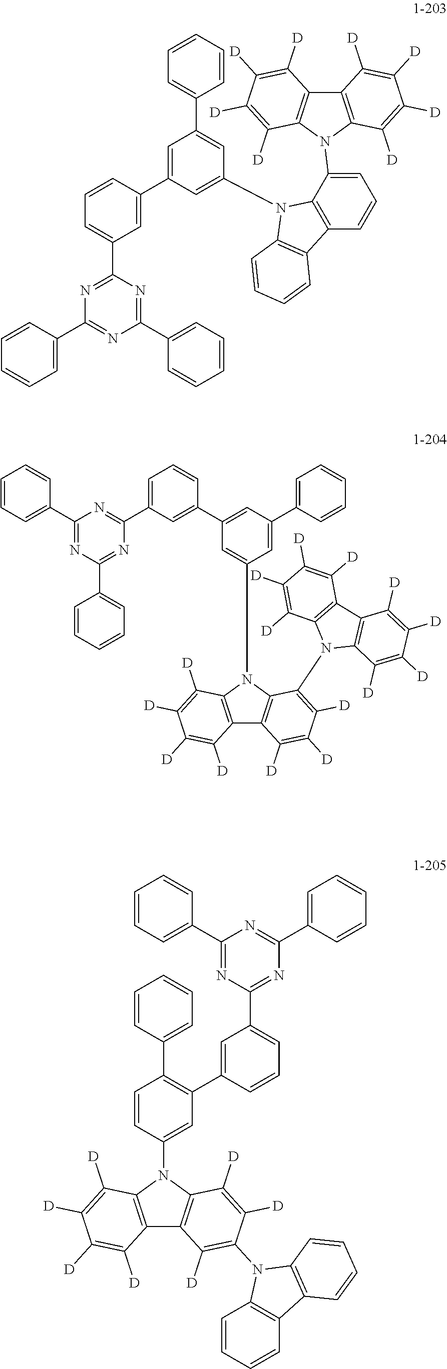 [SMILES: [2H]c1c([2H])c([2H])c2c(c1[2H])c1c([2H])c([2H])c([2H])c([2H])c1n2-c1cccc2c3ccccc3n(-c3cc(-c4ccccc4)cc(-c4cccc(-c5nc(-c6ccccc6)nc(-c6ccccc6)n5)c4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2)cc(-c2cccc(-c3nc(-c4ccccc4)nc(-c4ccccc4)n3)c2)c1 ; [2H]c1c([2H])c([2H])c2c(c1[2H])c1c([2H])c(-n3c4ccccc4c4ccccc43)c([2H])c([2H])c1n2-c1ccc(-c2ccccc2)c(-c2cccc(-c3nc(-c4ccccc4)nc(-c4ccccc4)n3)c2)c1]

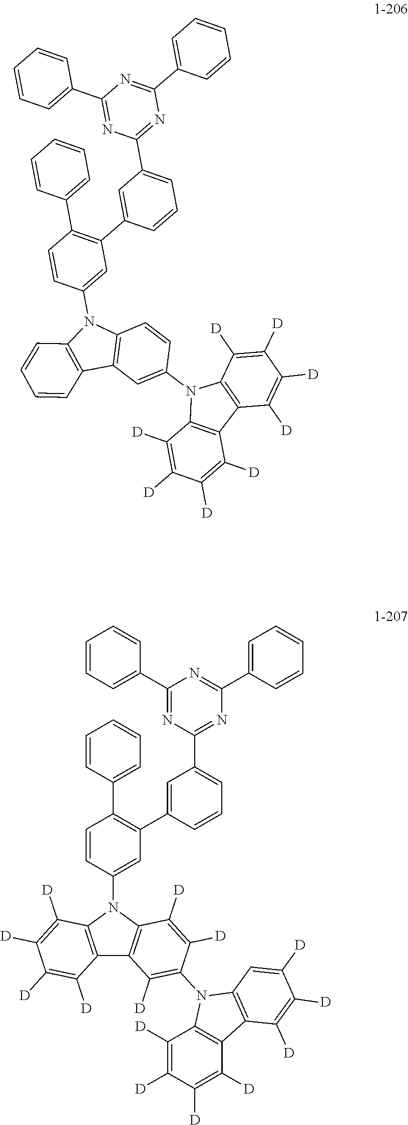 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2ccccc2)c(-c2cccc(-c3nc(-c4ccccc4)nc(-c4ccccc4)n3)c2)c1 ; [2H]c1cc2c(c([2H])c1[2H])c1c([2H])c([2H])c([2H])c([2H])c1n2-c1c([2H])c([2H])c2c(c1[2H])c1c([2H])c([2H])c([2H])c([2H])c1n2-c1ccc(-c2ccccc2)c(-c2cccc(-c3nc(-c4ccccc4)nc(-c4ccccc4)n3)c2)c1]

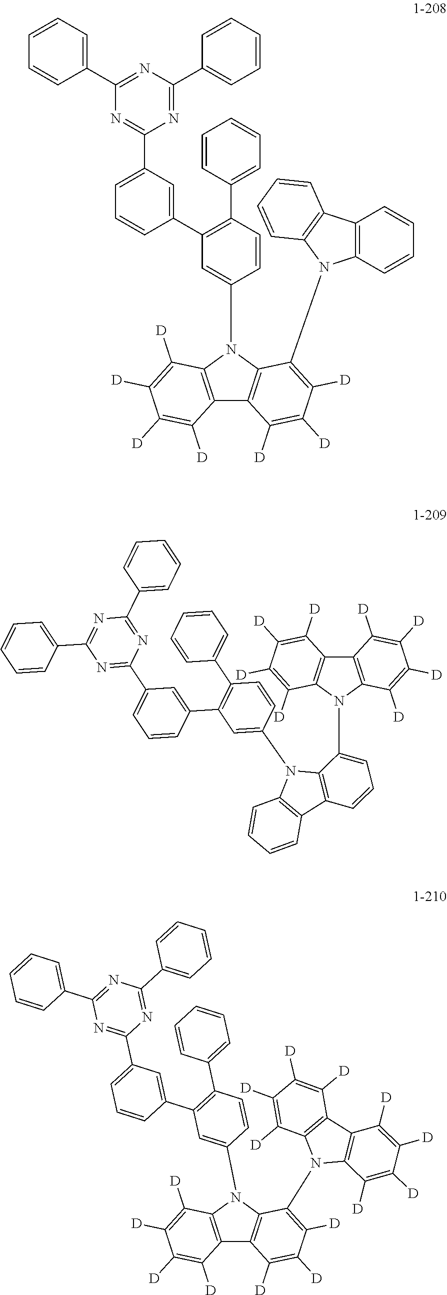 [SMILES: [2H]c1c([2H])c([2H])c2c(c1[2H])c1c([2H])c([2H])c([2H])c(-n3c4ccccc4c4ccccc43)c1n2-c1ccc(-c2ccccc2)c(-c2cccc(-c3nc(-c4ccccc4)nc(-c4ccccc4)n3)c2)c1 ; [2H]c1c([2H])c([2H])c2c(c1[2H])c1c([2H])c([2H])c([2H])c([2H])c1n2-c1cccc2c3ccccc3n(-c3ccc(-c4ccccc4)c(-c4cccc(-c5nc(-c6ccccc6)nc(-c6ccccc6)n5)c4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)c(-c2cccc(-c3nc(-c4ccccc4)nc(-c4ccccc4)n3)c2)c1]

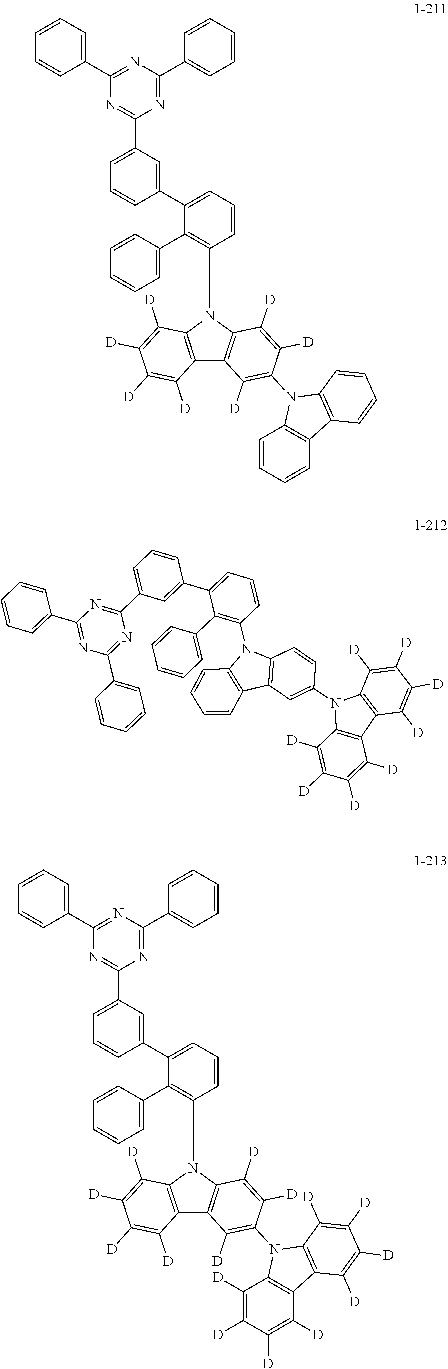 [SMILES: [2H]c1c([2H])c([2H])c2c(c1[2H])c1c([2H])c(-n3c4ccccc4c4ccccc43)c([2H])c([2H])c1n2-c1cccc(-c2cccc(-c3nc(-c4ccccc4)nc(-c4ccccc4)n3)c2)c1-c1ccccc1 ; [2H]c1c([2H])c([2H])c2c(c1[2H])c1c([2H])c([2H])c([2H])c([2H])c1n2-c1ccc2c(c1)c1ccccc1n2-c1cccc(-c2cccc(-c3nc(-c4ccccc4)nc(-c4ccccc4)n3)c2)c1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cccc(-c3nc(-c4ccccc4)nc(-c4ccccc4)n3)c2)c1-c1ccccc1]

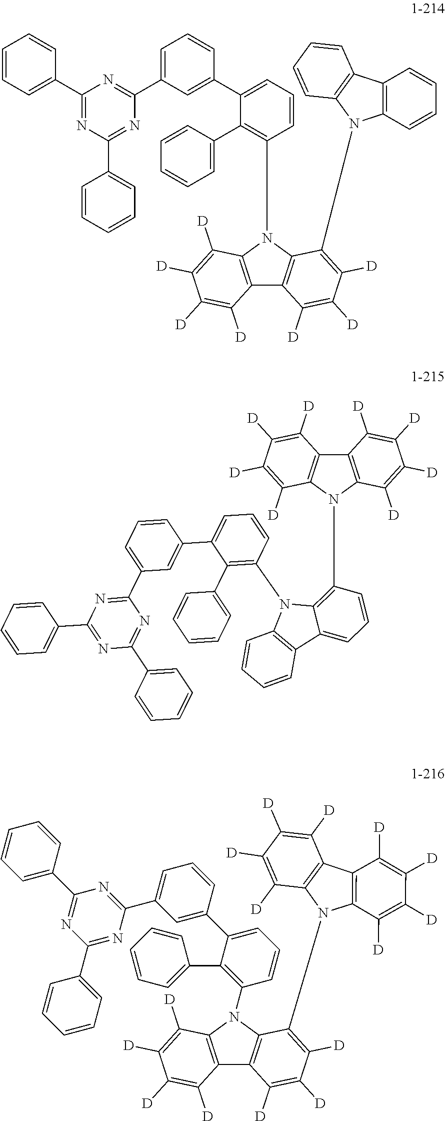 [SMILES: [2H]c1c([2H])c([2H])c2c(c1[2H])c1c([2H])c([2H])c([2H])c(-n3c4ccccc4c4ccccc43)c1n2-c1cccc(-c2cccc(-c3nc(-c4ccccc4)nc(-c4ccccc4)n3)c2)c1-c1ccccc1 ; [2H]c1c([2H])c([2H])c2c(c1[2H])c1c([2H])c([2H])c([2H])c([2H])c1n2-c1cccc2c3ccccc3n(-c3cccc(-c4cccc(-c5nc(-c6ccccc6)nc(-c6ccccc6)n5)c4)c3-c3ccccc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cccc(-c3nc(-c4ccccc4)nc(-c4ccccc4)n3)c2)c1-c1ccccc1]

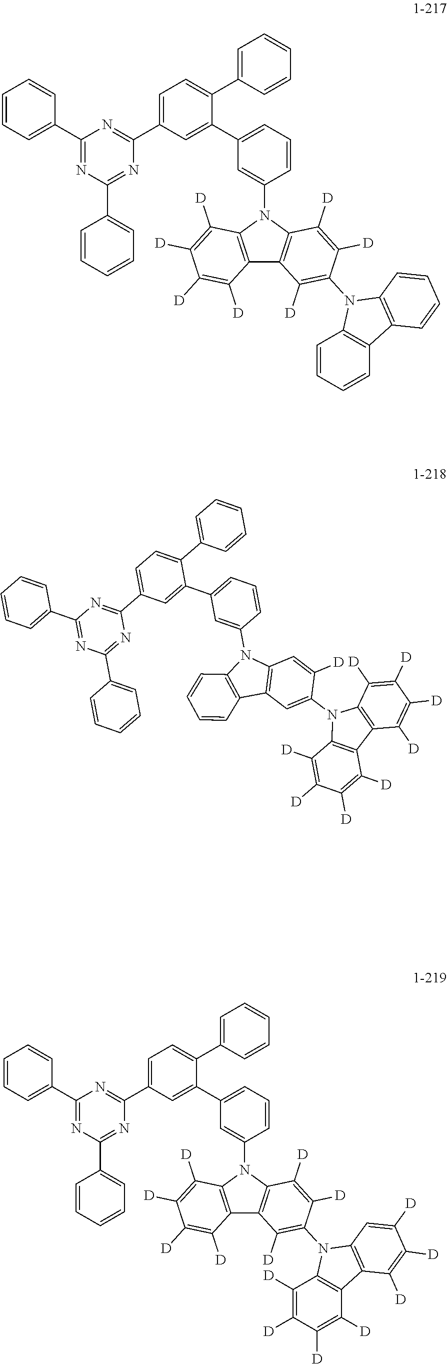 [SMILES: [2H]c1c([2H])c([2H])c2c(c1[2H])c1c([2H])c(-n3c4ccccc4c4ccccc43)c([2H])c([2H])c1n2-c1cccc(-c2cc(-c3nc(-c4ccccc4)nc(-c4ccccc4)n3)ccc2-c2ccccc2)c1 ; [2H]c1cc2c(cc1-n1c3c([2H])c([2H])c([2H])c([2H])c3c3c([2H])c([2H])c([2H])c([2H])c31)c1ccccc1n2-c1cccc(-c2cc(-c3nc(-c4ccccc4)nc(-c4ccccc4)n3)ccc2-c2ccccc2)c1 ; [2H]c1cc2c(c([2H])c1[2H])c1c([2H])c([2H])c([2H])c([2H])c1n2-c1c([2H])c([2H])c2c(c1[2H])c1c([2H])c([2H])c([2H])c([2H])c1n2-c1cccc(-c2cc(-c3nc(-c4ccccc4)nc(-c4ccccc4)n3)ccc2-c2ccccc2)c1]

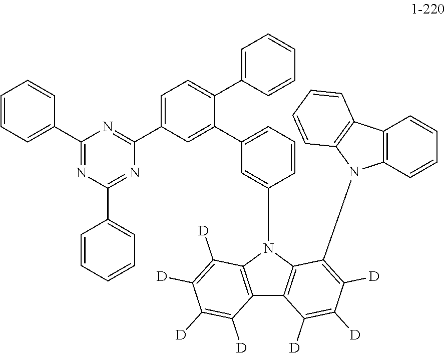 [SMILES: [2H]c1c([2H])c([2H])c2c(c1[2H])c1c([2H])c([2H])c([2H])c(-n3c4ccccc4c4ccccc43)c1n2-c1cccc(-c2cc(-c3nc(-c4ccccc4)nc(-c4ccccc4)n3)ccc2-c2ccccc2)c1]

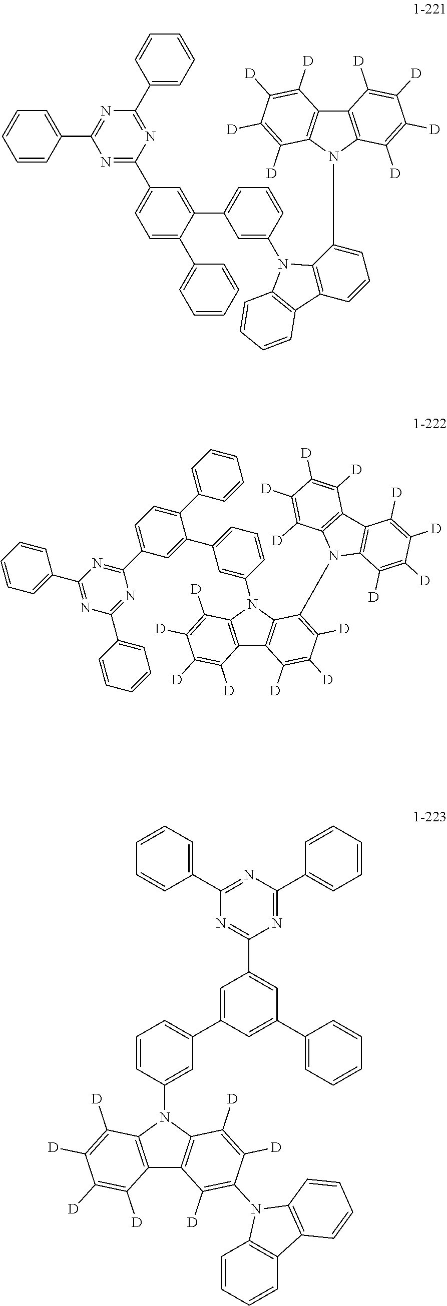 [SMILES: [2H]c1c([2H])c([2H])c2c(c1[2H])c1c([2H])c([2H])c([2H])c([2H])c1n2-c1cccc2c3ccccc3n(-c3cccc(-c4cc(-c5nc(-c6ccccc6)nc(-c6ccccc6)n5)ccc4-c4ccccc4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cc(-c3nc(-c4ccccc4)nc(-c4ccccc4)n3)ccc2-c2ccccc2)c1 ; [2H]c1c([2H])c([2H])c2c(c1[2H])c1c([2H])c(-n3c4ccccc4c4ccccc43)c([2H])c([2H])c1n2-c1cccc(-c2cc(-c3ccccc3)cc(-c3nc(-c4ccccc4)nc(-c4ccccc4)n3)c2)c1]

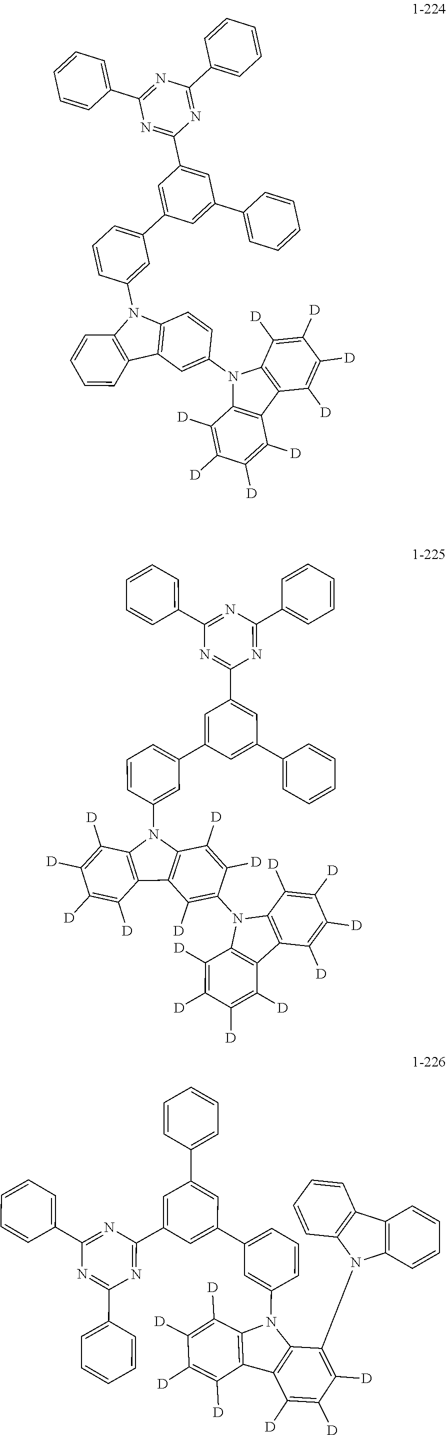 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2cc(-c3ccccc3)cc(-c3nc(-c4ccccc4)nc(-c4ccccc4)n3)c2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cc(-c3ccccc3)cc(-c3nc(-c4ccccc4)nc(-c4ccccc4)n3)c2)c1 ; [2H]c1c([2H])c([2H])c2c(c1[2H])c1c([2H])c([2H])c([2H])c(-n3c4ccccc4c4ccccc43)c1n2-c1cccc(-c2cc(-c3ccccc3)cc(-c3nc(-c4ccccc4)nc(-c4ccccc4)n3)c2)c1]

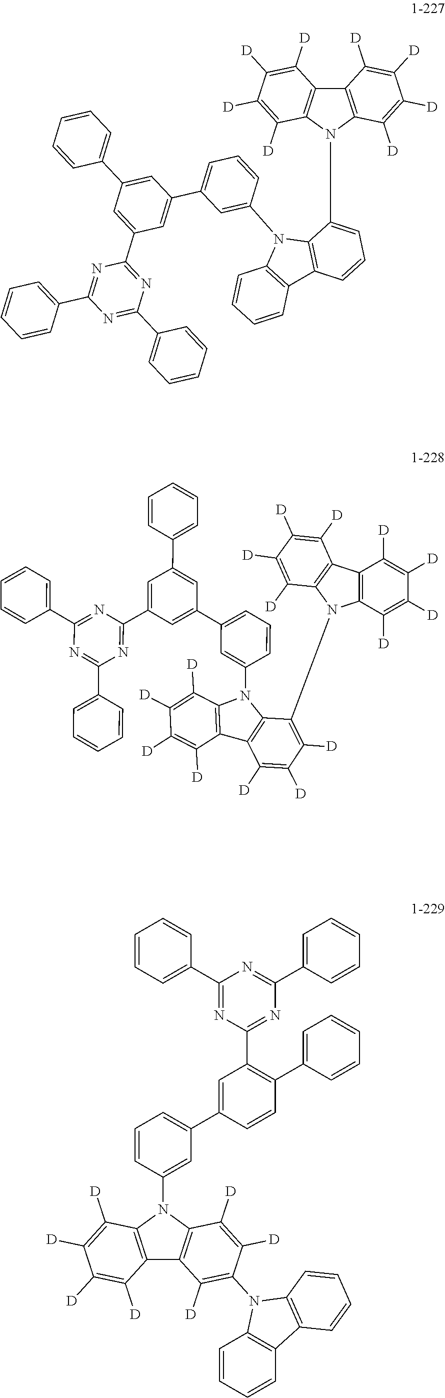 [SMILES: [2H]c1c([2H])c([2H])c2c(c1[2H])c1c([2H])c([2H])c([2H])c([2H])c1n2-c1cccc2c3ccccc3n(-c3cccc(-c4cc(-c5ccccc5)cc(-c5nc(-c6ccccc6)nc(-c6ccccc6)n5)c4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cc(-c3ccccc3)cc(-c3nc(-c4ccccc4)nc(-c4ccccc4)n3)c2)c1 ; [2H]c1c([2H])c([2H])c2c(c1[2H])c1c([2H])c(-n3c4ccccc4c4ccccc43)c([2H])c([2H])c1n2-c1cccc(-c2ccc(-c3ccccc3)c(-c3nc(-c4ccccc4)nc(-c4ccccc4)n3)c2)c1]

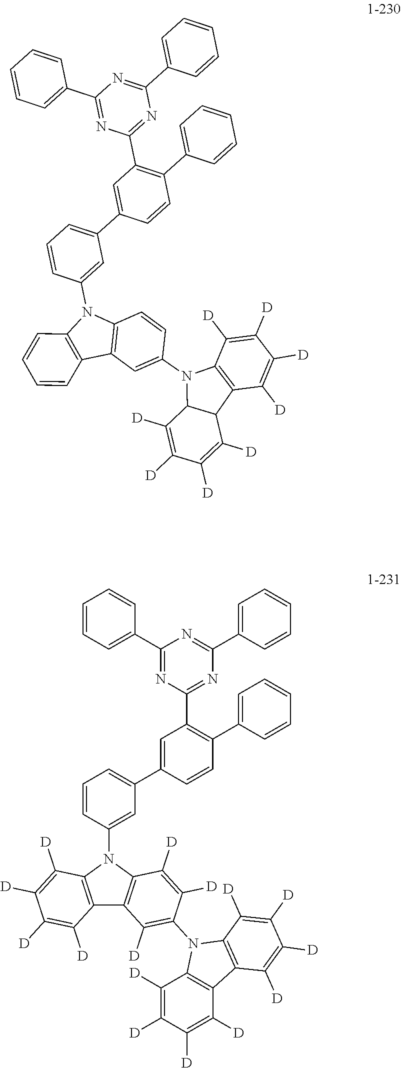 [SMILES: [2H]C1=C([2H])C2c3c([2H])c([2H])c([2H])c([2H])c3N(c3ccc4c(c3)c3ccccc3n4-c3cccc(-c4ccc(-c5ccccc5)c(-c5nc(-c6ccccc6)nc(-c6ccccc6)n5)c4)c3)C2C([2H])=C1[2H] ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccc(-c3ccccc3)c(-c3nc(-c4ccccc4)nc(-c4ccccc4)n3)c2)c1]

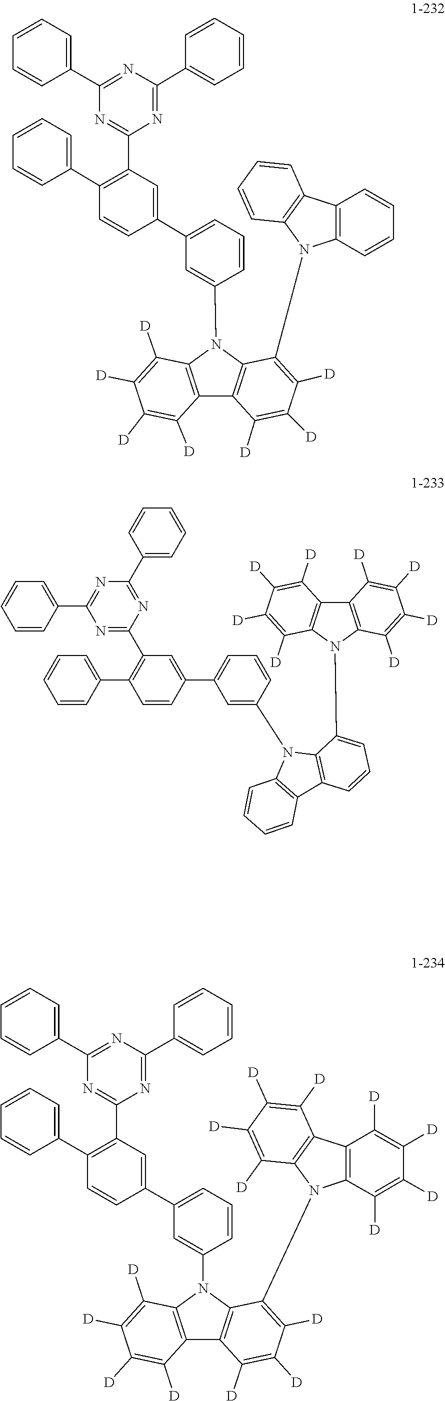

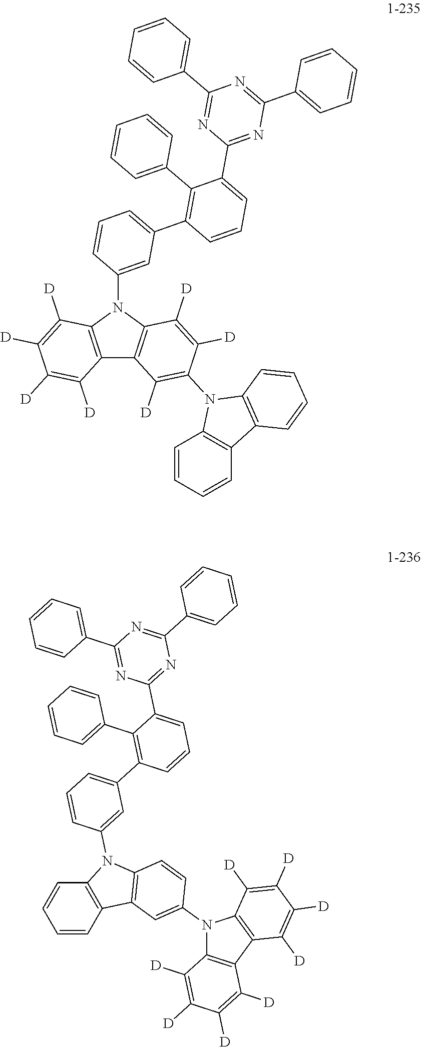

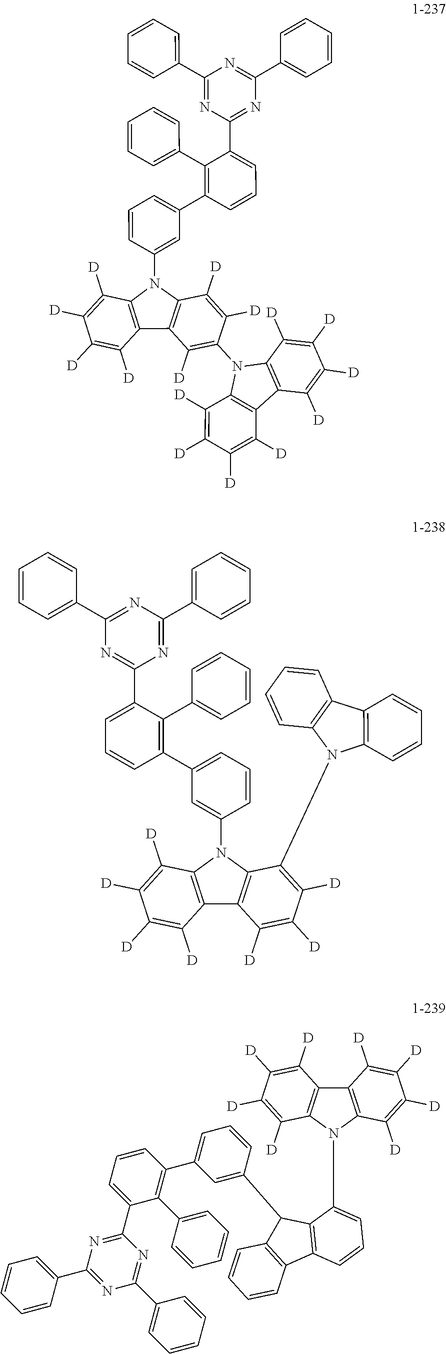 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cccc(-c3nc(-c4ccccc4)nc(-c4ccccc4)n3)c2-c2ccccc2)c1 ; [2H]c1c([2H])c([2H])c2c(c1[2H])c1c([2H])c([2H])c([2H])c(-n3c4ccccc4c4ccccc43)c1n2-c1cccc(-c2cccc(-c3nc(-c4ccccc4)nc(-c4ccccc4)n3)c2-c2ccccc2)c1 ; [2H]c1c([2H])c([2H])c2c(c1[2H])c1c([2H])c([2H])c([2H])c([2H])c1n2-c1cccc2c1C(c1cccc(-c3cccc(-c4nc(-c5ccccc5)nc(-c5ccccc5)n4)c3-c3ccccc3)c1)c1ccccc1-2]

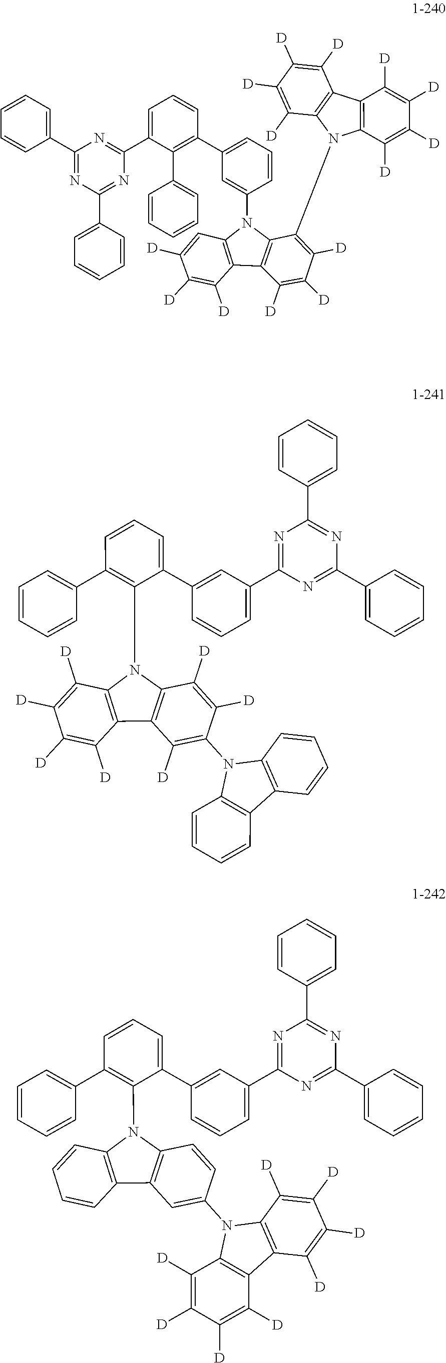 [SMILES: [2H]c1cc2c(c([2H])c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cccc(-c3nc(-c4ccccc4)nc(-c4ccccc4)n3)c2-c2ccccc2)c1 ; [2H]c1c([2H])c([2H])c2c(c1[2H])c1c([2H])c(-n3c4ccccc4c4ccccc43)c([2H])c([2H])c1n2-c1c(-c2ccccc2)cccc1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1c(-c2ccccc2)cccc1-c1cccc(-c2nc(-c3ccccc3)nc(-c3ccccc3)n2)c1]

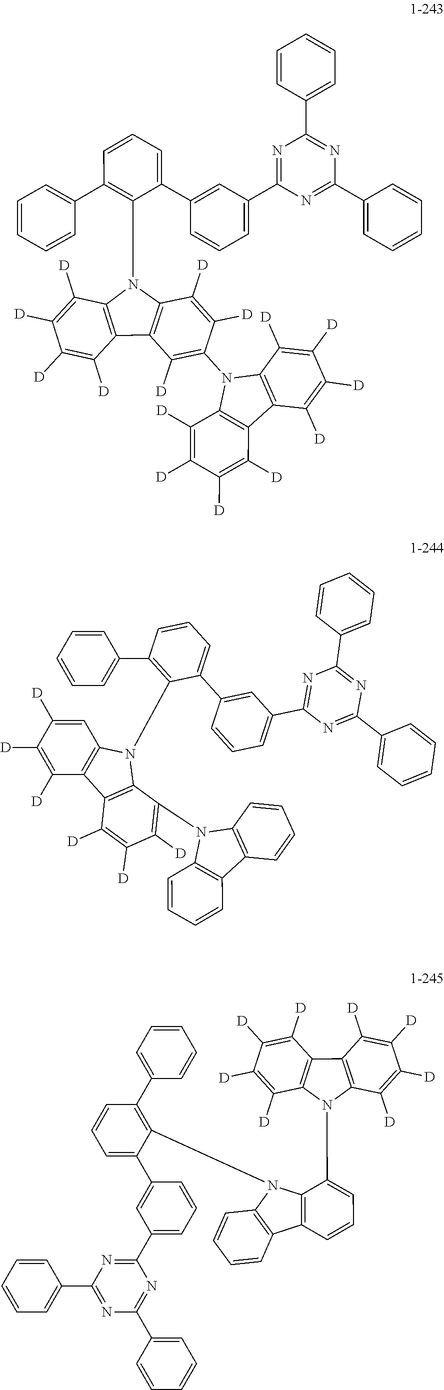 [SMILES: [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1c(-c2ccccc2)cccc1-c1cccc(-c2nc(-c3ccccc3)nc(-c3ccccc3)n2)c1 ; [2H]c1cc2c(c([2H])c1[2H])c1c([2H])c([2H])c([2H])c(-n3c4ccccc4c4ccccc43)c1n2-c1c(-c2ccccc2)cccc1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3c(-c4ccccc4)cccc3-c3cccc(-c4nc(-c5ccccc5)nc(-c5ccccc5)n4)c3)c12]

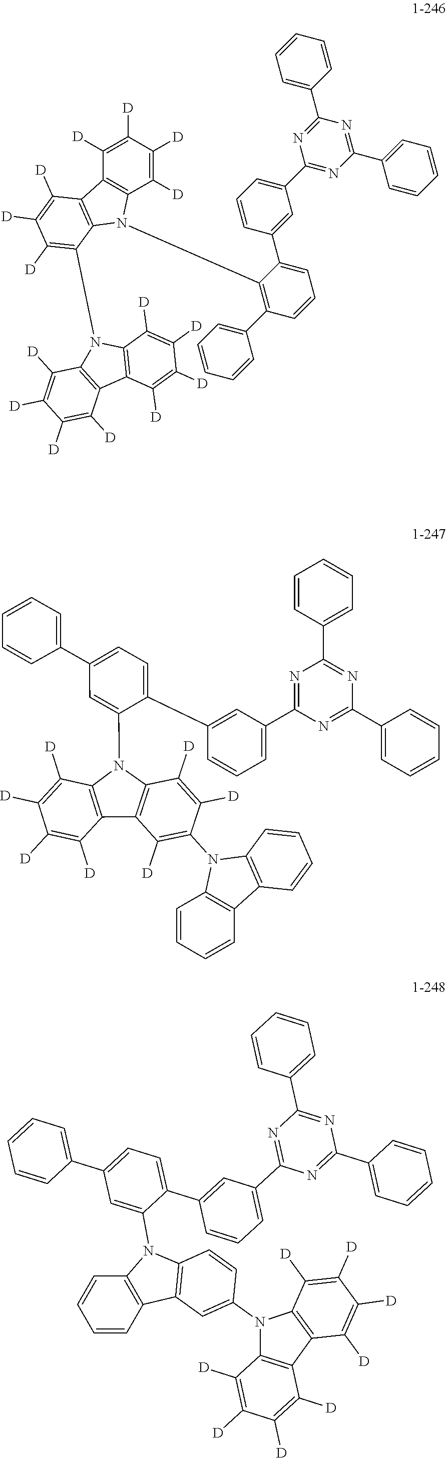 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1c(-c2ccccc2)cccc1-c1cccc(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cc(-c2ccccc2)ccc1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2)ccc1-c1cccc(-c2nc(-c3ccccc3)nc(-c3ccccc3)n2)c1]

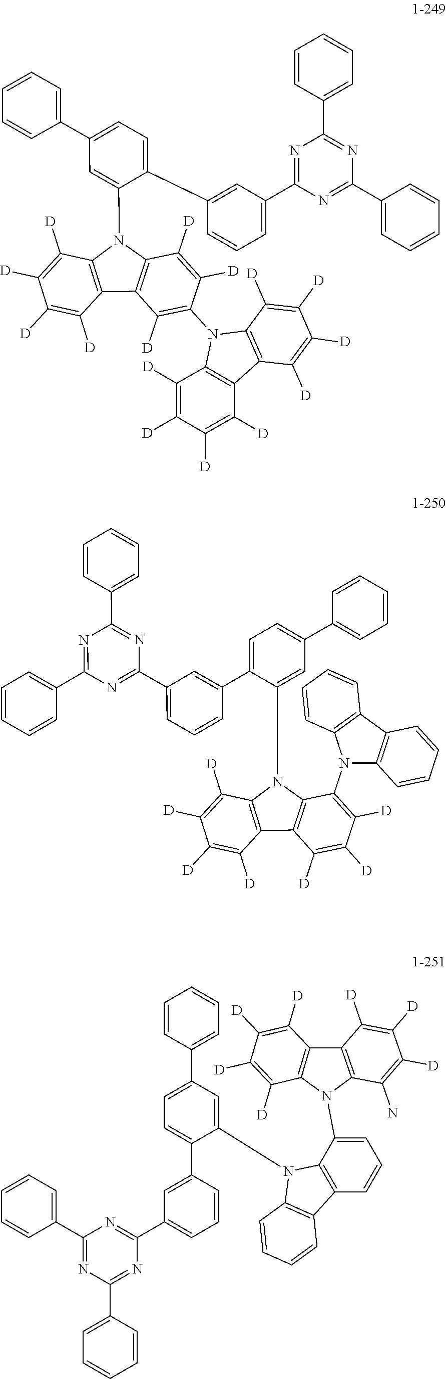 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)ccc1-c1cccc(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cc(-c2ccccc2)ccc1-c1cccc(-c2nc(-c3ccccc3)nc(-c3ccccc3)n2)c1 ; [2H]c1c([2H])c([2H])c2c(c1[2H])c1c([2H])c([2H])c([2H])c(N)c1n2-c1cccc2c3ccccc3n(-c3cc(-c4ccccc4)ccc3-c3cccc(-c4nc(-c5ccccc5)nc(-c5ccccc5)n4)c3)c12]

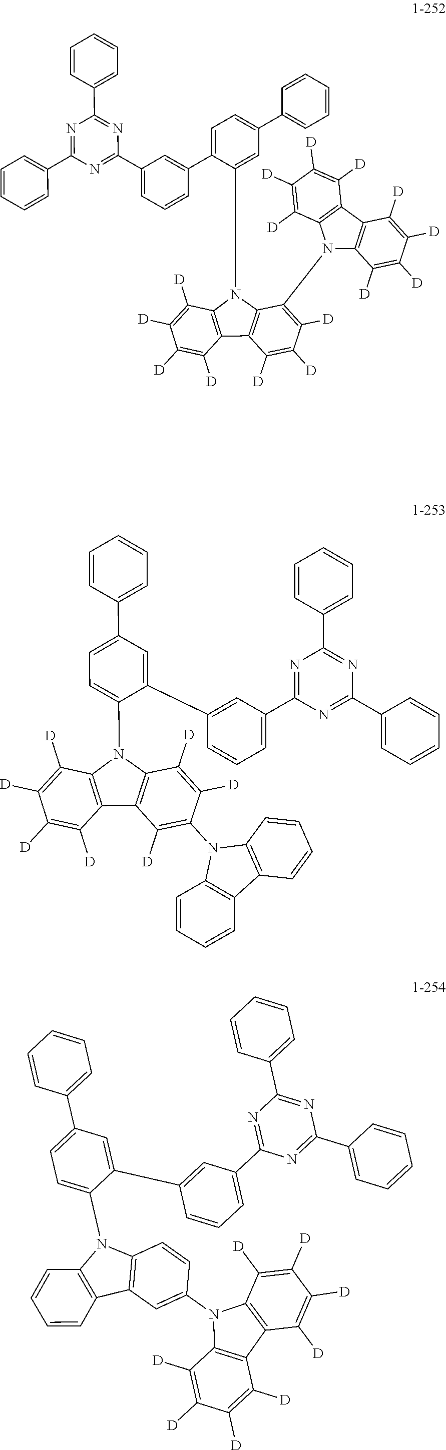 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2)ccc1-c1cccc(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1ccc(-c2ccccc2)cc1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1ccc(-c2ccccc2)cc1-c1cccc(-c2nc(-c3ccccc3)nc(-c3ccccc3)n2)c1]

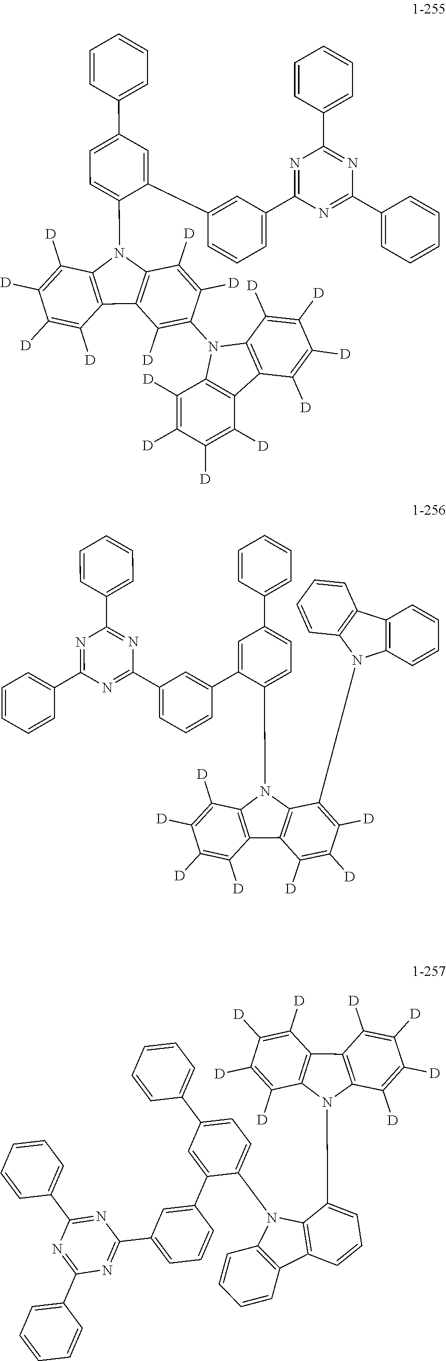 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)cc1-c1cccc(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1ccc(-c2ccccc2)cc1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3ccc(-c4ccccc4)cc3-c3cccc(-c4nc(-c5ccccc5)nc(-c5ccccc5)n4)c3)c12]

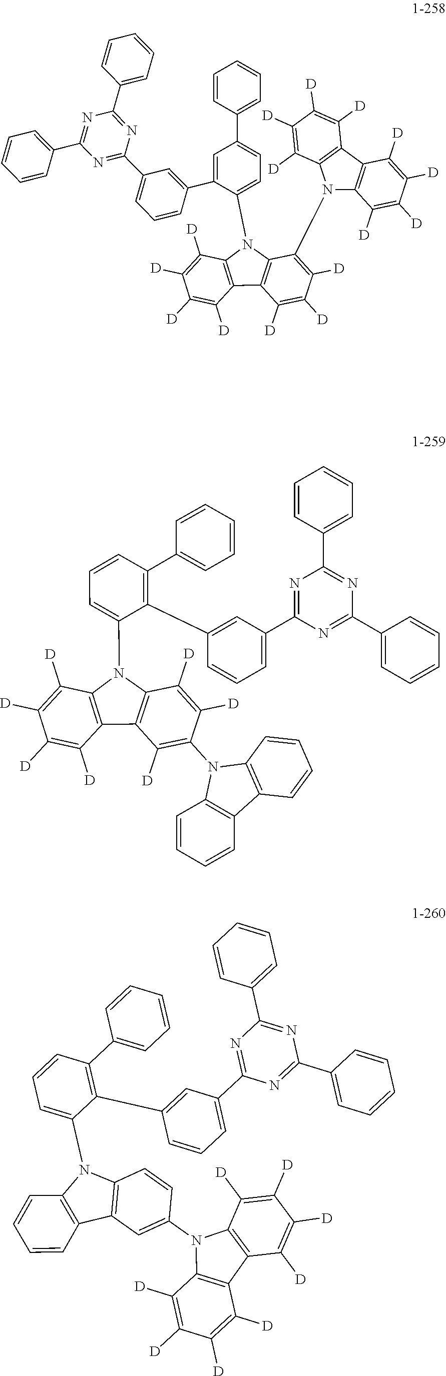 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)cc1-c1cccc(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cccc(-c2ccccc2)c1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1cccc(-c2ccccc2)c1-c1cccc(-c2nc(-c3ccccc3)nc(-c3ccccc3)n2)c1]

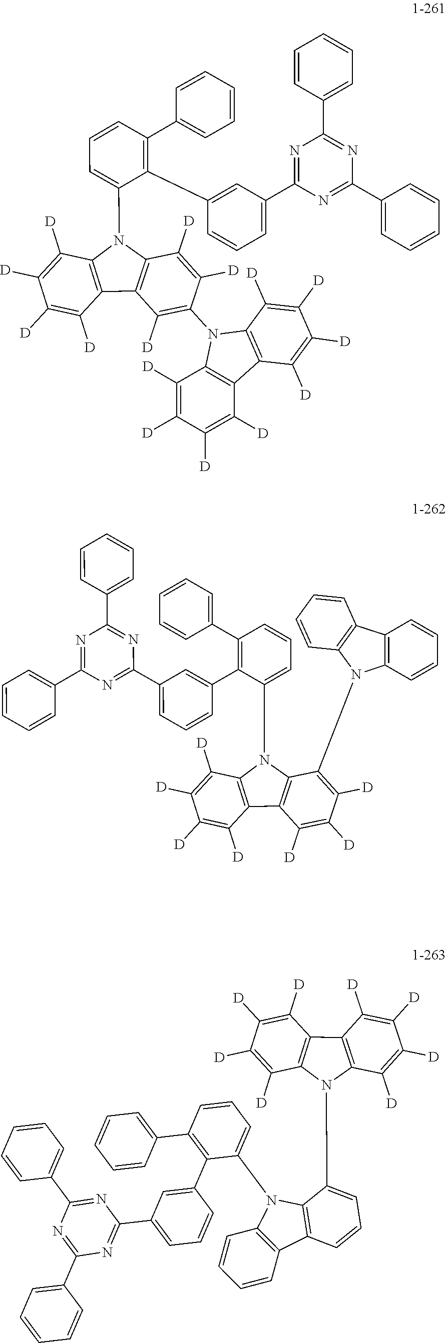 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccccc2)c1-c1cccc(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cccc(-c2ccccc2)c1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3cccc(-c4ccccc4)c3-c3cccc(-c4nc(-c5ccccc5)nc(-c5ccccc5)n4)c3)c12]

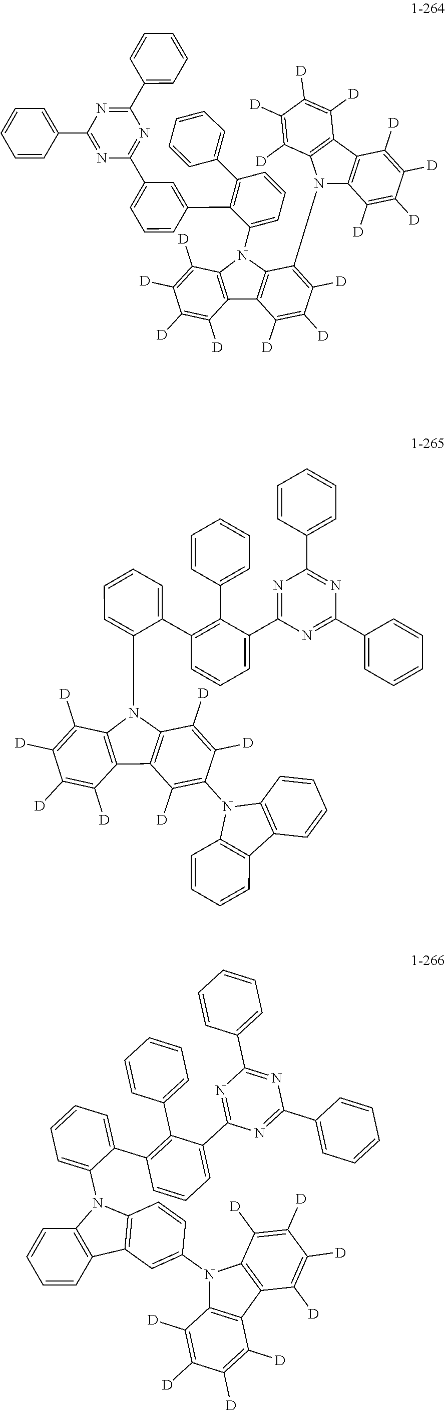 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccccc2)c1-c1cccc(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1ccccc1-c1cccc(-c2nc(-c3ccccc3)nc(-c3ccccc3)n2)c1-c1ccccc1 ; [2H]c1c([2H])c([2H])c2c(c1[2H])c1c([2H])c([2H])c([2H])c([2H])c1n2-c1ccc2c(c1)c1ccccc1n2-c1ccccc1-c1cccc(-c2nc(-c3ccccc3)nc(-c3ccccc3)n2)c1-c1ccccc1]

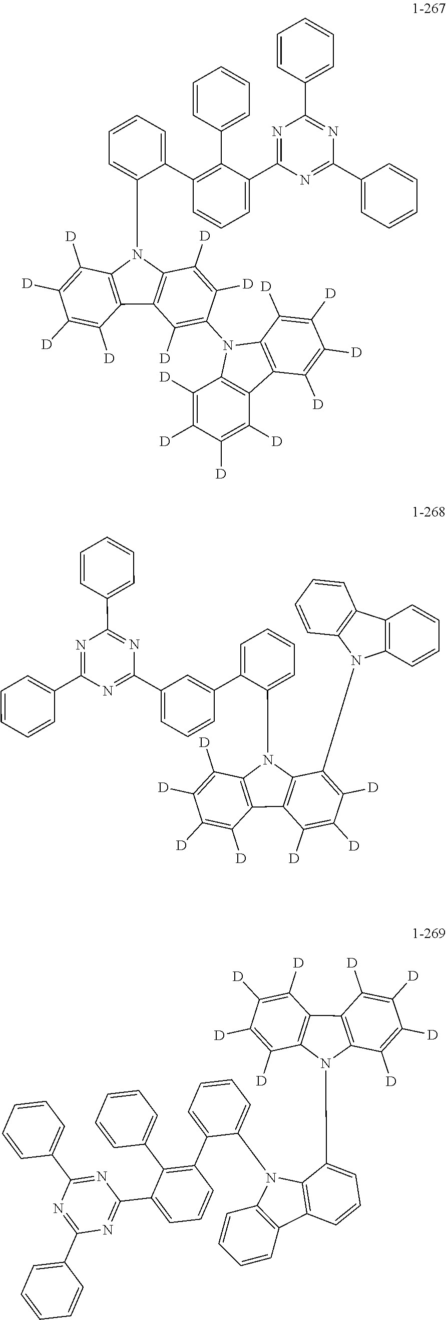 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1cccc(-c2nc(-c3ccccc3)nc(-c3ccccc3)n2)c1-c1ccccc1 ; [2H]c1c([2H])c([2H])c2c(c1[2H])c1c([2H])c([2H])c([2H])c(-n3c4ccccc4c4ccccc43)c1n2-c1ccccc1-c1cccc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3ccccc3-c3cccc(-c4nc(-c5ccccc5)nc(-c5ccccc5)n4)c3-c3ccccc3)c12]

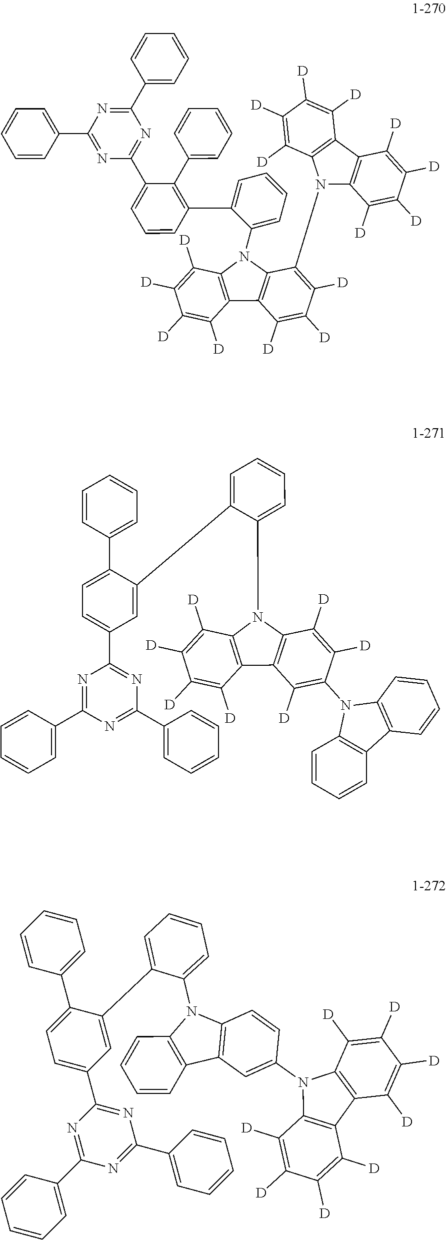 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1cccc(-c2nc(-c3ccccc3)nc(-c3ccccc3)n2)c1-c1ccccc1 ; [2H]c1c([2H])c([2H])c2c(c1[2H])c1c([2H])c(-n3c4ccccc4c4ccccc43)c([2H])c([2H])c1n2-c1ccccc1-c1cc(-c2nc(-c3ccccc3)nc(-c3ccccc3)n2)ccc1-c1ccccc1 ; [2H]c1c([2H])c([2H])c2c(c1[2H])c1c([2H])c([2H])c([2H])c([2H])c1n2-c1ccc2c(c1)c1ccccc1n2-c1ccccc1-c1cc(-c2nc(-c3ccccc3)nc(-c3ccccc3)n2)ccc1-c1ccccc1]

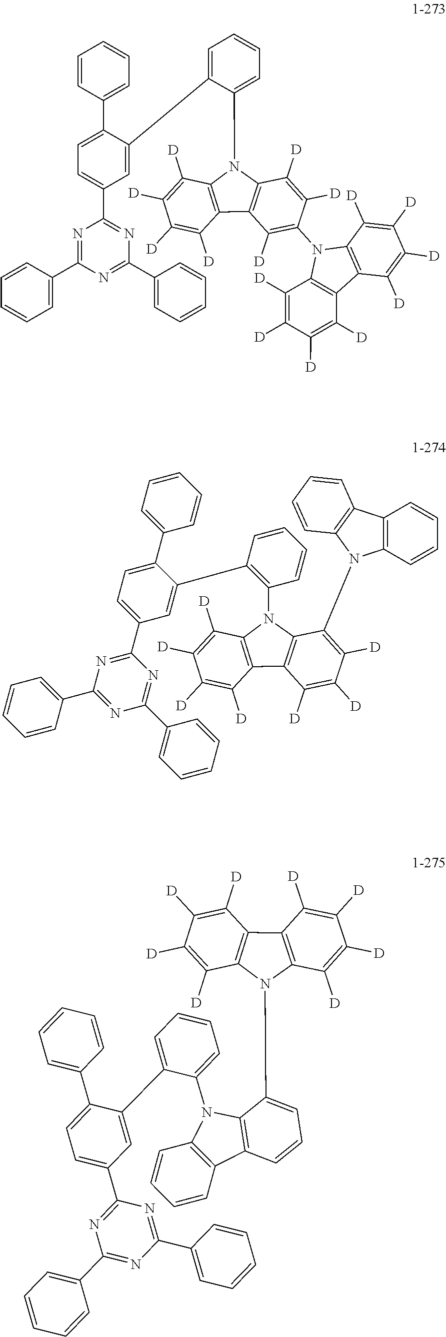 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1cc(-c2nc(-c3ccccc3)nc(-c3ccccc3)n2)ccc1-c1ccccc1 ; [2H]c1c([2H])c([2H])c2c(c1[2H])c1c([2H])c([2H])c([2H])c(-n3c4ccccc4c4ccccc43)c1n2-c1ccccc1-c1cc(-c2nc(-c3ccccc3)nc(-c3ccccc3)n2)ccc1-c1ccccc1 ; [2H]c1c([2H])c([2H])c2c(c1[2H])c1c([2H])c([2H])c([2H])c([2H])c1n2-c1cccc2c3ccccc3n(-c3ccccc3-c3cc(-c4nc(-c5ccccc5)nc(-c5ccccc5)n4)ccc3-c3ccccc3)c12]

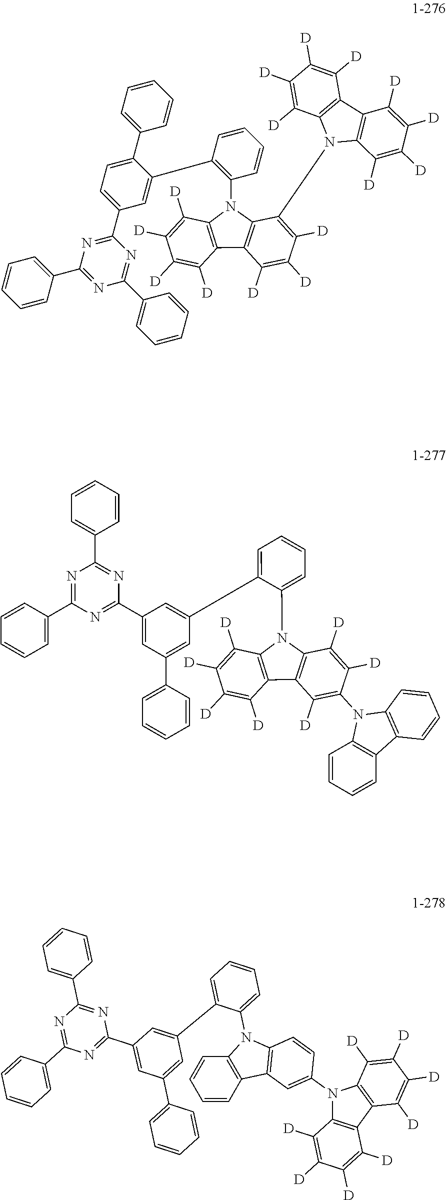 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1cc(-c2nc(-c3ccccc3)nc(-c3ccccc3)n2)ccc1-c1ccccc1 ; [2H]c1c([2H])c([2H])c2c(c1[2H])c1c([2H])c(-n3c4ccccc4c4ccccc43)c([2H])c([2H])c1n2-c1ccccc1-c1cc(-c2ccccc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1ccccc1-c1cc(-c2ccccc2)cc(-c2nc(-c3ccccc3)nc(-c3ccccc3)n2)c1]

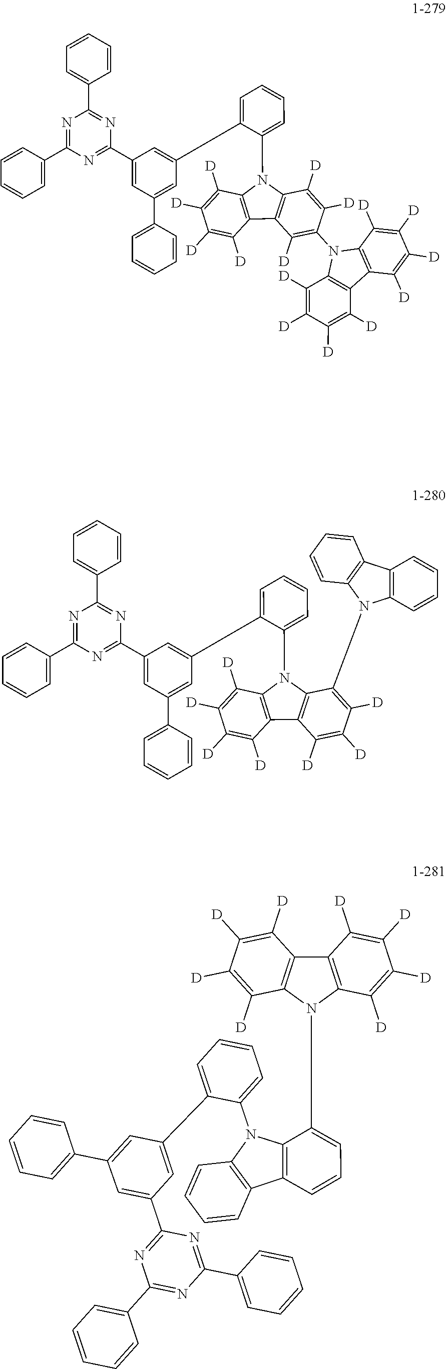 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1cc(-c2ccccc2)cc(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1ccccc1-c1cc(-c2ccccc2)cc(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3ccccc3-c3cc(-c4ccccc4)cc(-c4nc(-c5ccccc5)nc(-c5ccccc5)n4)c3)c12]

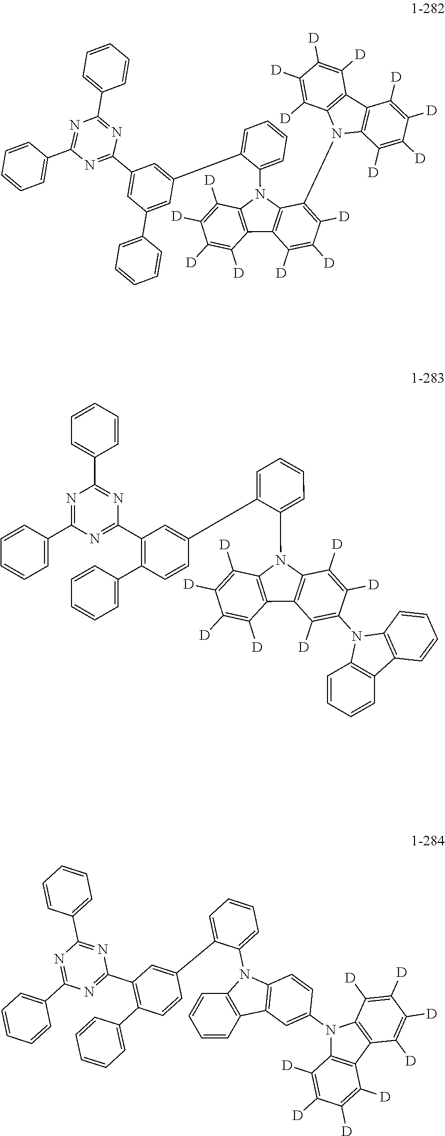 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1cc(-c2ccccc2)cc(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1ccccc1-c1ccc(-c2ccccc2)c(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1ccccc1-c1ccc(-c2ccccc2)c(-c2nc(-c3ccccc3)nc(-c3ccccc3)n2)c1]

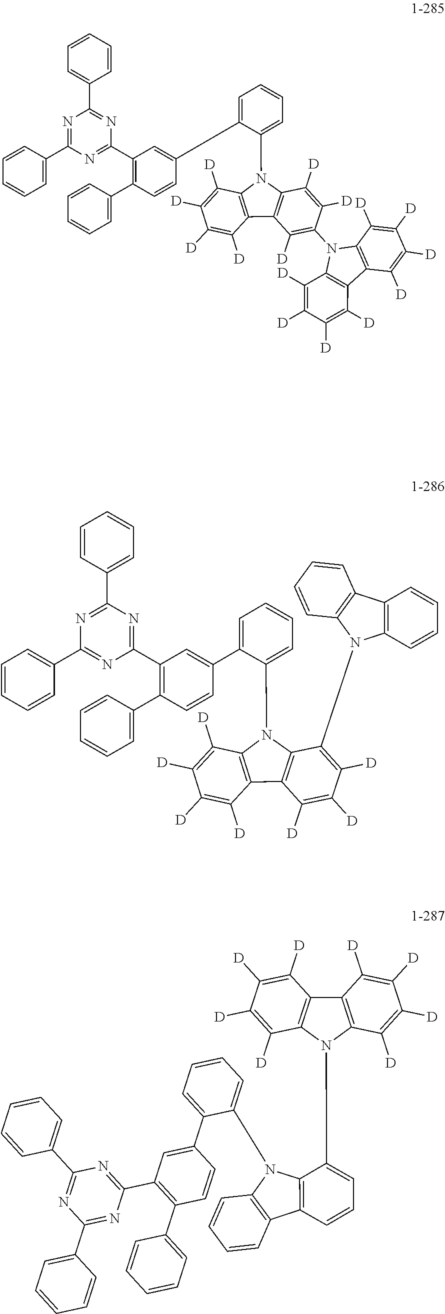 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1ccc(-c2ccccc2)c(-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1ccccc1-c1ccc(-c2ccccc2)c(-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3ccccc3-c3ccc(-c4ccccc4)c(-c4nc(-c5ccccc5)nc(-c5ccccc5)n4)c3)c12]

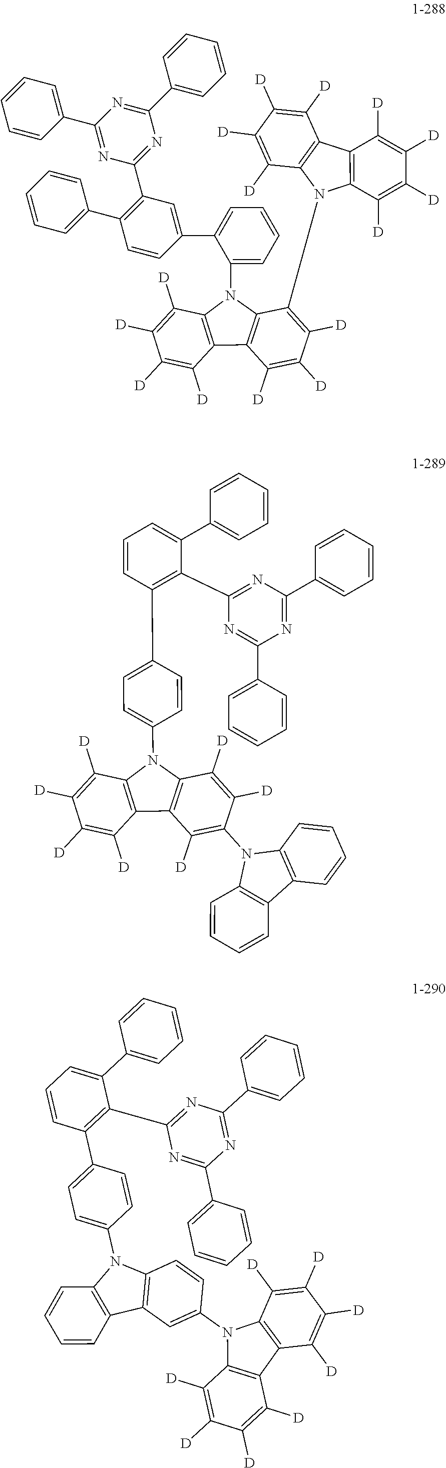 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1ccc(-c2ccccc2)c(-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1ccc(-c2cccc(-c3ccccc3)c2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2cccc(-c3ccccc3)c2-c2nc(-c3ccccc3)nc(-c3ccccc3)n2)cc1]

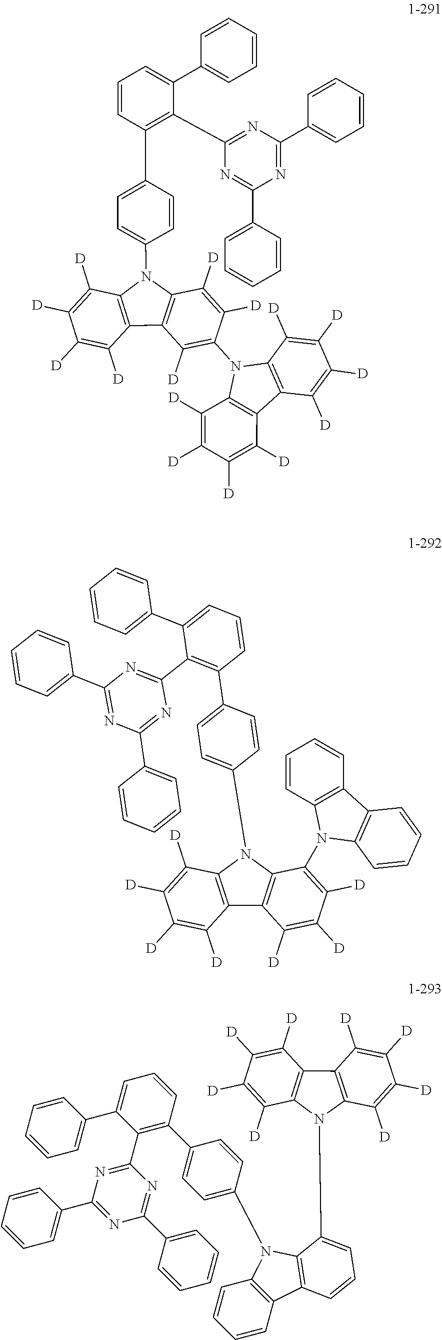 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cccc(-c3ccccc3)c2-c2nc(-c3ccccc3)nc(-c3ccccc3)n2)cc1 ; [2H]c1c([2H])c([2H])c2c(c1[2H])c1c([2H])c([2H])c([2H])c(-n3c4ccccc4c4ccccc43)c1n2-c1ccc(-c2cccc(-c3ccccc3)c2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1cccc2c3ccccc3n(-c3ccc(-c4cccc(-c5ccccc5)c4-c4nc(-c5ccccc5)nc(-c5ccccc5)n4)cc3)c12]

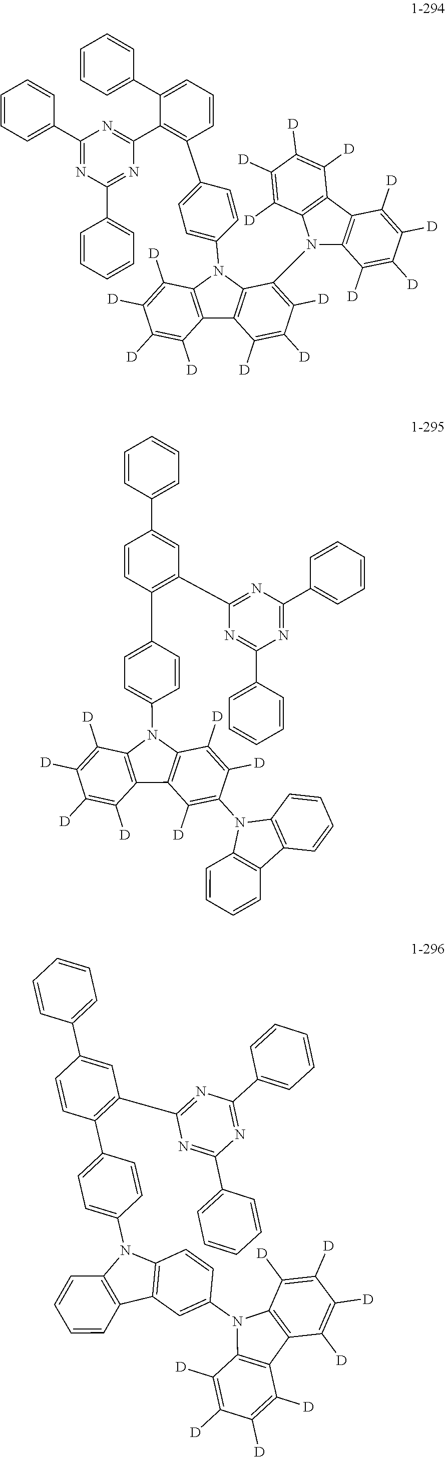 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2cccc(-c3ccccc3)c2-c2nc(-c3ccccc3)nc(-c3ccccc3)n2)cc1 ; [2H]c1c([2H])c([2H])c2c(c1[2H])c1c([2H])c(-n3c4ccccc4c4ccccc43)c([2H])c([2H])c1n2-c1ccc(-c2ccc(-c3ccccc3)cc2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2ccc(-c3ccccc3)cc2-c2nc(-c3ccccc3)nc(-c3ccccc3)n2)cc1]

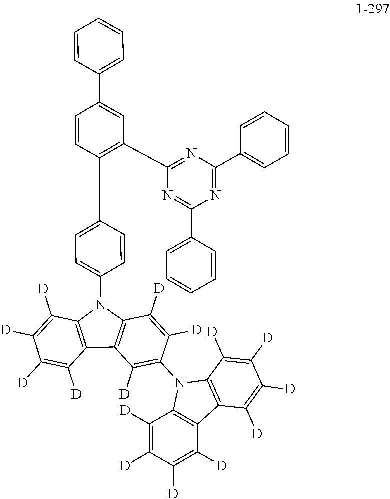 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3ccccc3)cc2-c2nc(-c3ccccc3)nc(-c3ccccc3)n2)cc1]

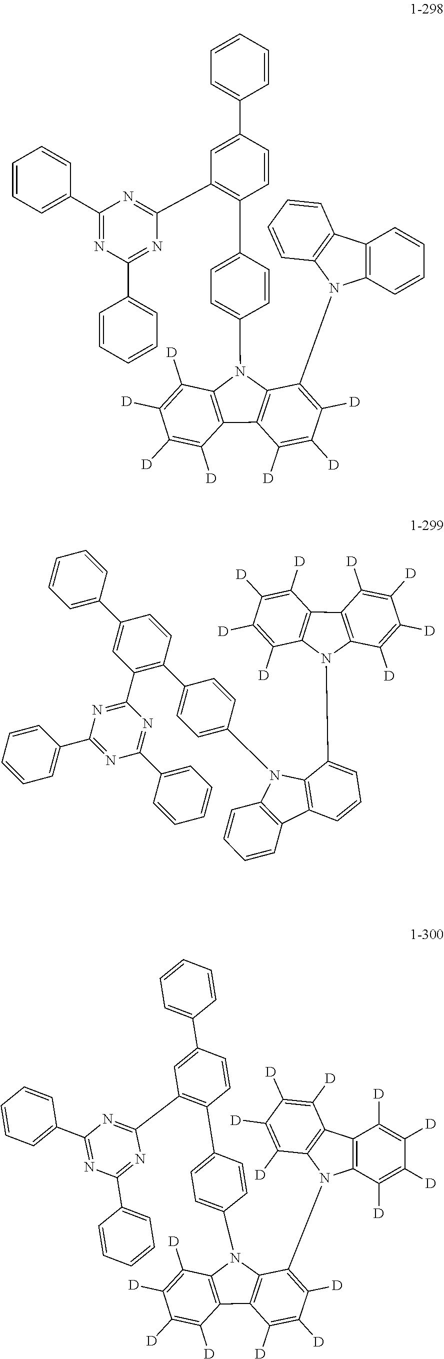 [SMILES: [2H]c1c([2H])c([2H])c2c(c1[2H])c1c([2H])c([2H])c([2H])c(-n3c4ccccc4c4ccccc43)c1n2-c1ccc(-c2ccc(-c3ccccc3)cc2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1cccc2c3ccccc3n(-c3ccc(-c4ccc(-c5ccccc5)cc4-c4nc(-c5ccccc5)nc(-c5ccccc5)n4)cc3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3ccccc3)cc2-c2nc(-c3ccccc3)nc(-c3ccccc3)n2)cc1]

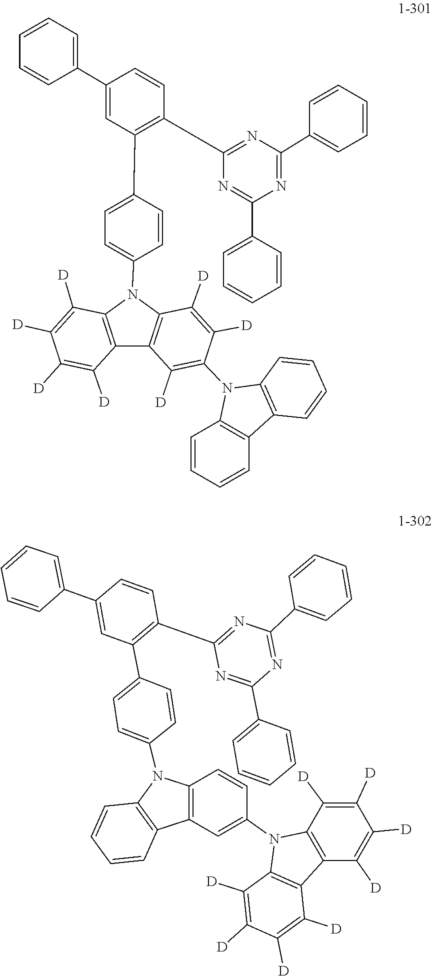 [SMILES: [2H]c1c([2H])c([2H])c2c(c1[2H])c1c([2H])c(-n3c4ccccc4c4ccccc43)c([2H])c([2H])c1n2-c1ccc(-c2cc(-c3ccccc3)ccc2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1ccc2c(c1)c1ccccc1n2-c1ccc(-c2cc(-c3ccccc3)ccc2-c2nc(-c3ccccc3)nc(-c3ccccc3)n2)cc1]

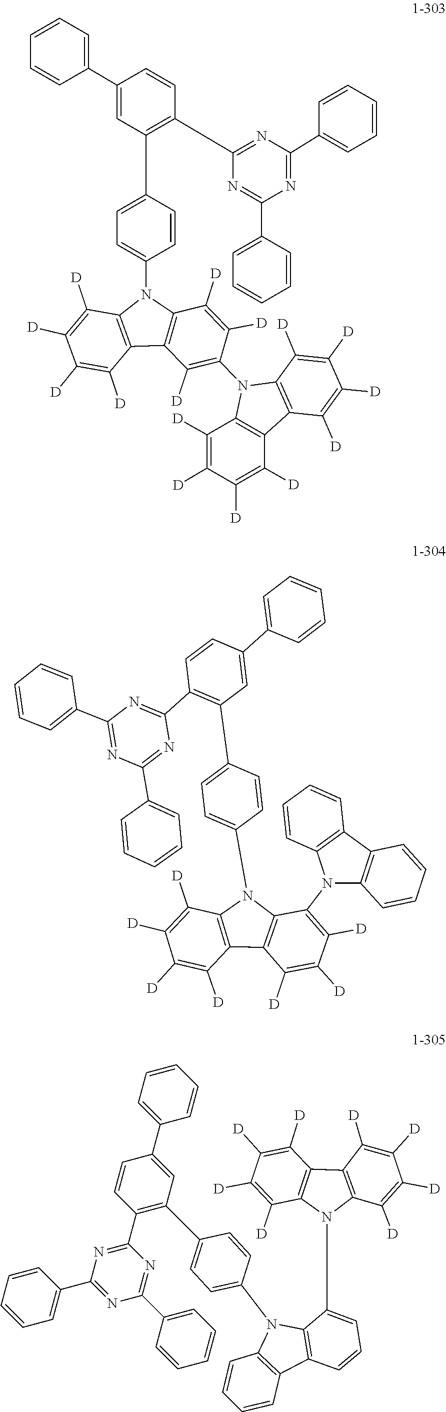 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2cc(-c3ccccc3)ccc2-c2nc(-c3ccccc3)nc(-c3ccccc3)n2)cc1 ; [2H]c1c([2H])c([2H])c2c(c1[2H])c1c([2H])c([2H])c([2H])c(-n3c4ccccc4c4ccccc43)c1n2-c1ccc(-c2cc(-c3ccccc3)ccc2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1cccc2c3ccccc3n(-c3ccc(-c4cc(-c5ccccc5)ccc4-c4nc(-c5ccccc5)nc(-c5ccccc5)n4)cc3)c12]

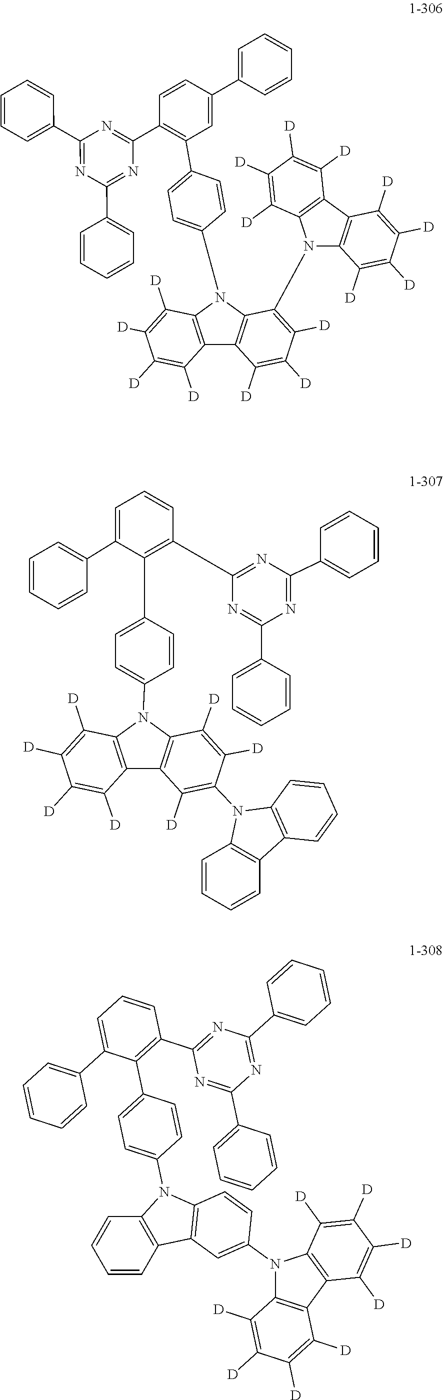

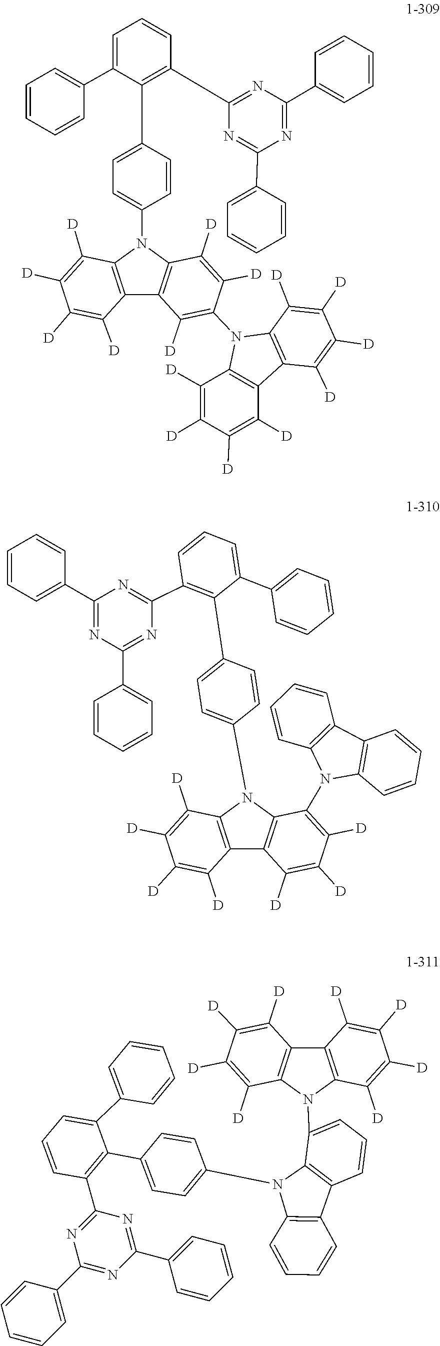 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2c(-c3ccccc3)cccc2-c2nc(-c3ccccc3)nc(-c3ccccc3)n2)cc1 ; [2H]c1c([2H])c([2H])c2c(c1[2H])c1c([2H])c([2H])c([2H])c(-n3c4ccccc4c4ccccc43)c1n2-c1ccc(-c2c(-c3ccccc3)cccc2-c2nc(-c3ccccc3)nc(-c3ccccc3)n2)cc1 ; [2H]c1c([2H])c([2H])c2c(c1[2H])c1c([2H])c([2H])c([2H])c([2H])c1n2-c1cccc2c3ccccc3n(-c3ccc(-c4c(-c5ccccc5)cccc4-c4nc(-c5ccccc5)nc(-c5ccccc5)n4)cc3)c12]

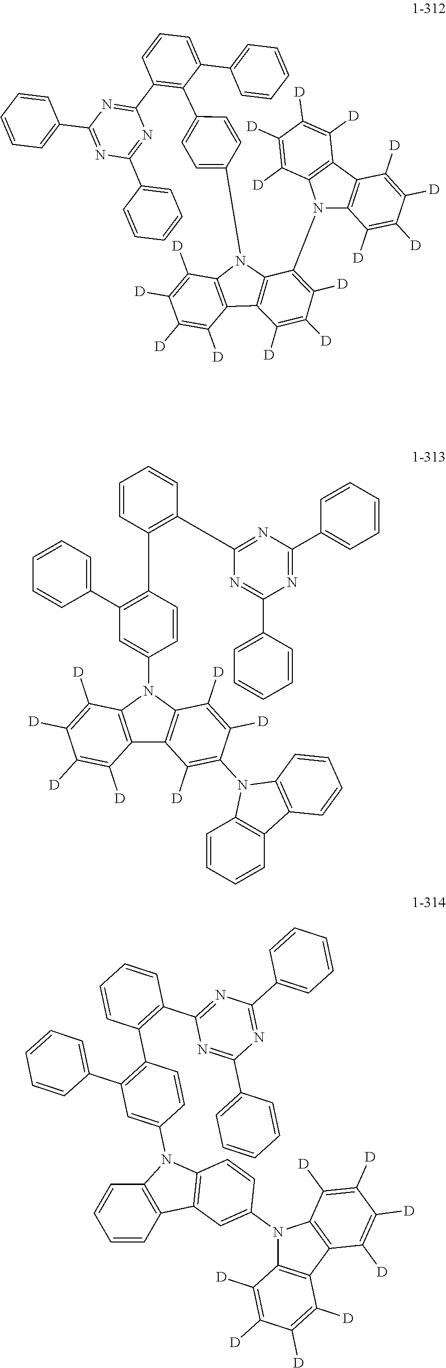

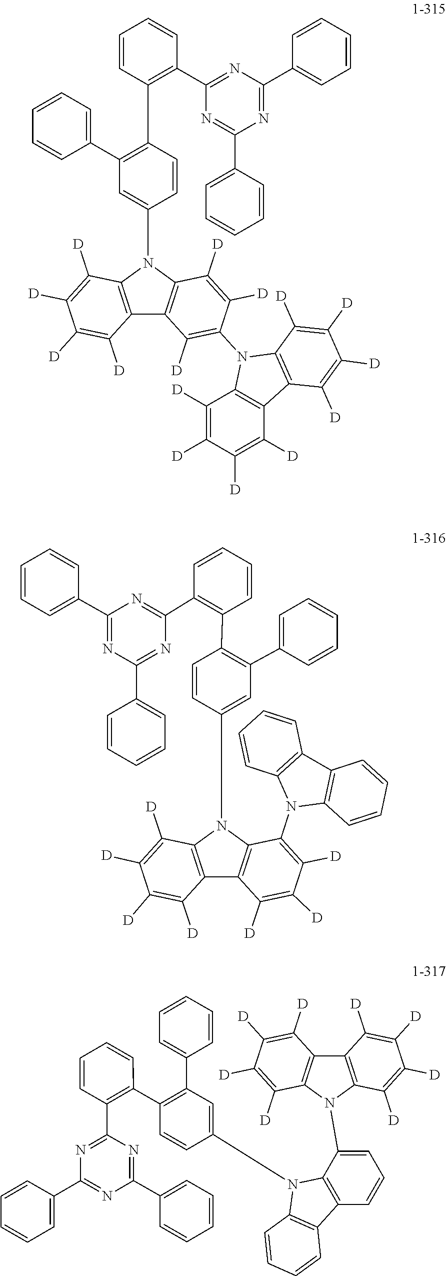

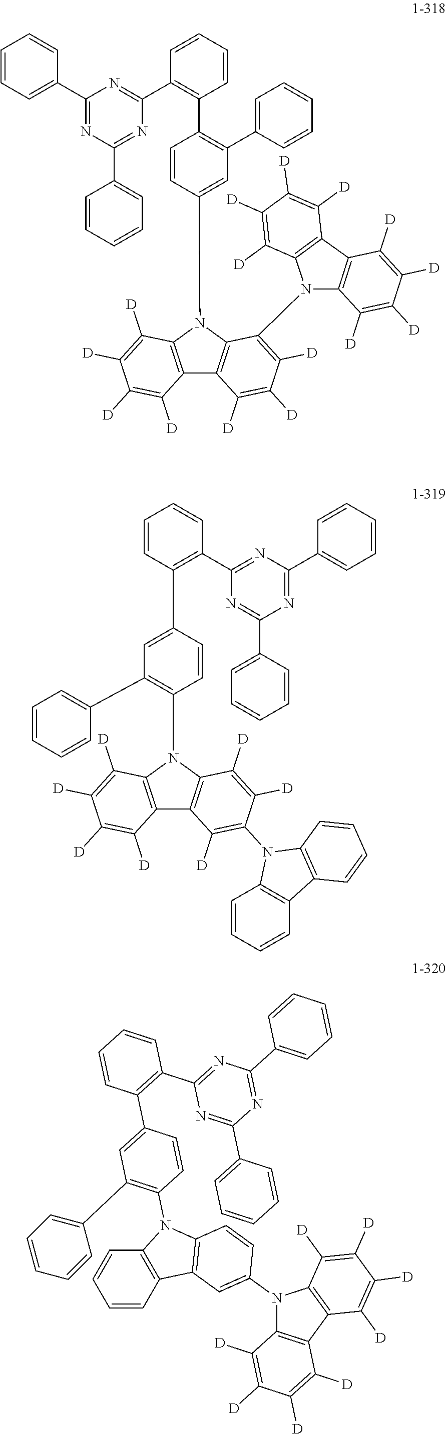

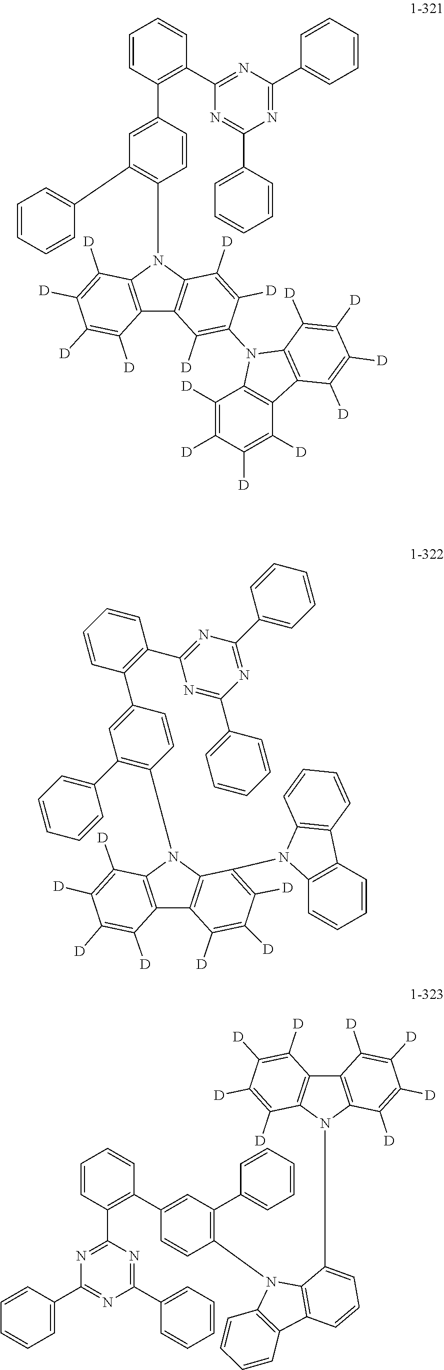

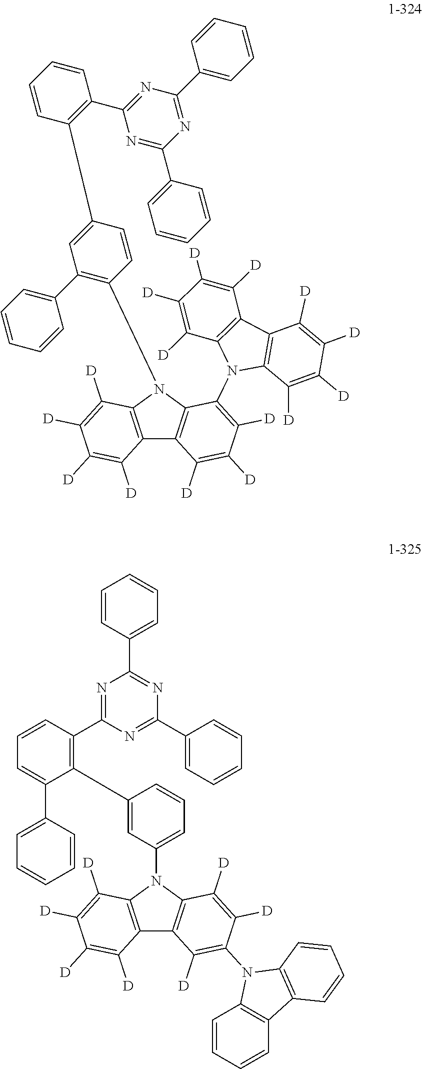 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2-c2nc(-c3ccccc3)nc(-c3ccccc3)n2)cc1-c1ccccc1 ; [2H]c1c([2H])c([2H])c2c(c1[2H])c1c([2H])c(-n3c4ccccc4c4ccccc43)c([2H])c([2H])c1n2-c1cccc(-c2c(-c3ccccc3)cccc2-c2nc(-c3ccccc3)nc(-c3ccccc3)n2)c1]

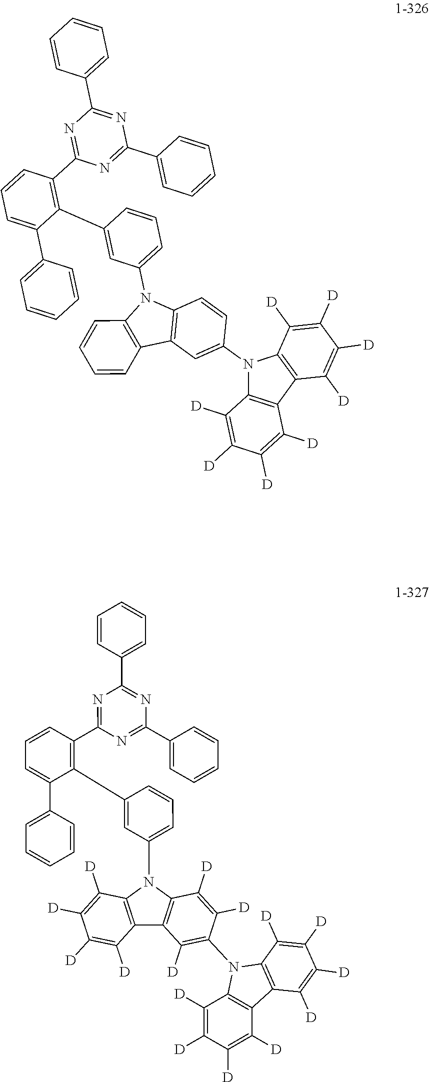 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2c(-c3ccccc3)cccc2-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2c(-c3ccccc3)cccc2-c2nc(-c3ccccc3)nc(-c3ccccc3)n2)c1]

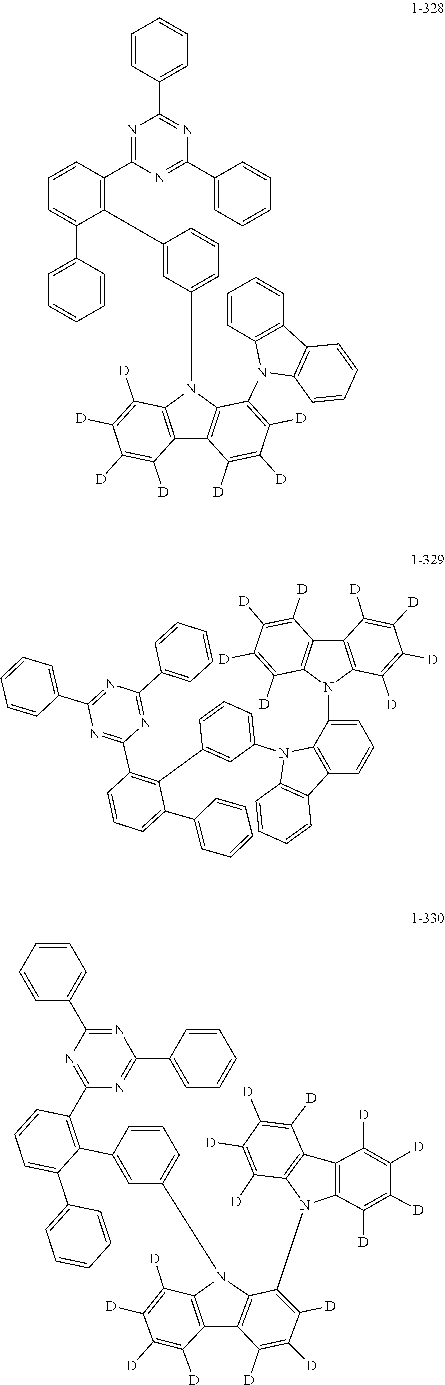

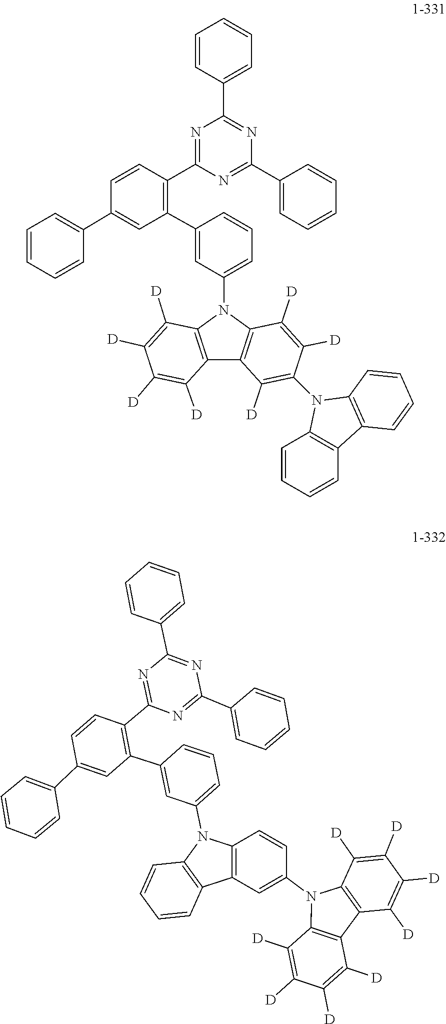 [SMILES: [2H]c1c([2H])c([2H])c2c(c1[2H])c1c([2H])c(-n3c4ccccc4c4ccccc43)c([2H])c([2H])c1n2-c1cccc(-c2cc(-c3ccccc3)ccc2-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1ccc2c(c1)c1ccccc1n2-c1cccc(-c2cc(-c3ccccc3)ccc2-c2nc(-c3ccccc3)nc(-c3ccccc3)n2)c1]

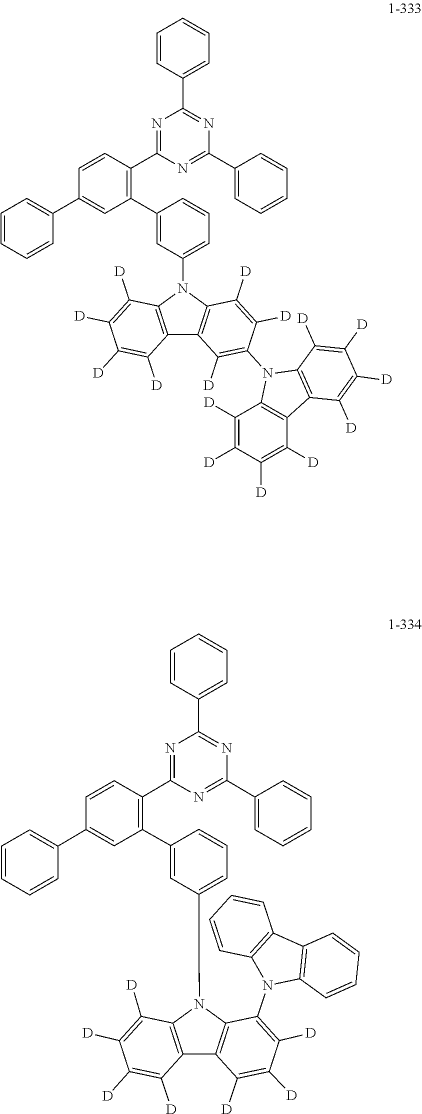 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cc(-c3ccccc3)ccc2-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1cccc(-c2cc(-c3ccccc3)ccc2-c2nc(-c3ccccc3)nc(-c3ccccc3)n2)c1]

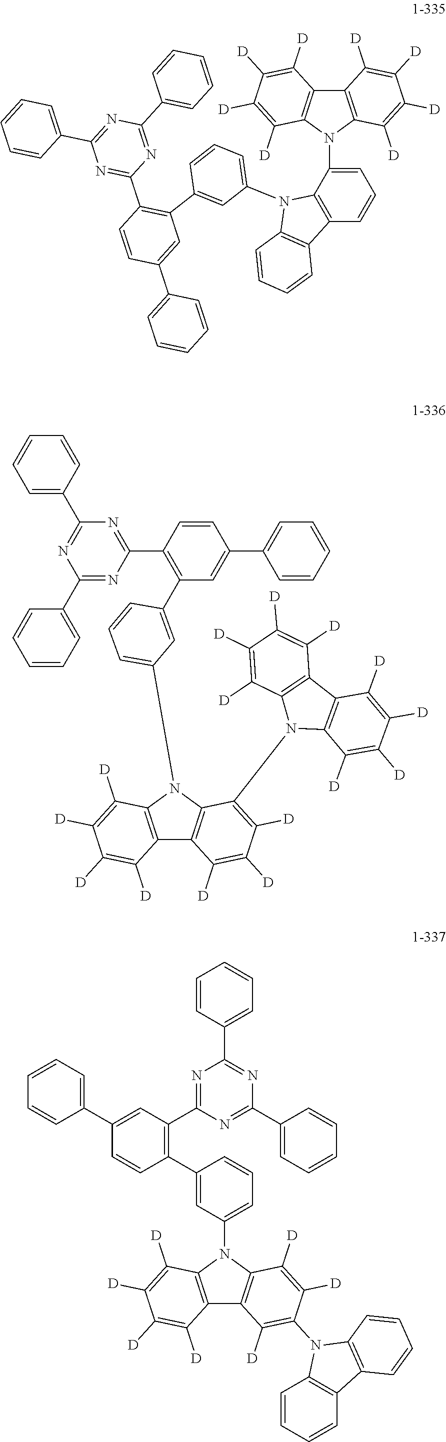 [SMILES: [2H]c1c([2H])c([2H])c2c(c1[2H])c1c([2H])c([2H])c([2H])c([2H])c1n2-c1cccc2c3ccccc3n(-c3cccc(-c4cc(-c5ccccc5)ccc4-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cc(-c3ccccc3)ccc2-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cccc(-c2ccc(-c3ccccc3)cc2-c2nc(-c3ccccc3)nc(-c3ccccc3)n2)c1]

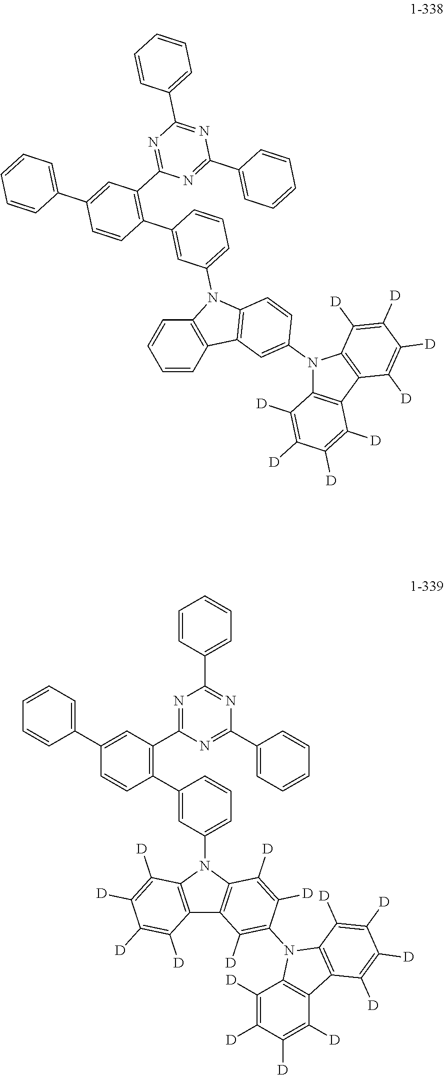

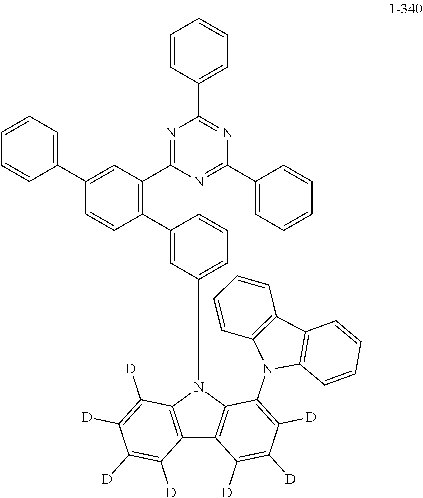 [SMILES: [2H]c1c([2H])c([2H])c2c(c1[2H])c1c([2H])c([2H])c([2H])c(-n3c4ccccc4c4ccccc43)c1n2-c1cccc(-c2ccc(-c3ccccc3)cc2-c2nc(-c3ccccc3)nc(-c3ccccc3)n2)c1]

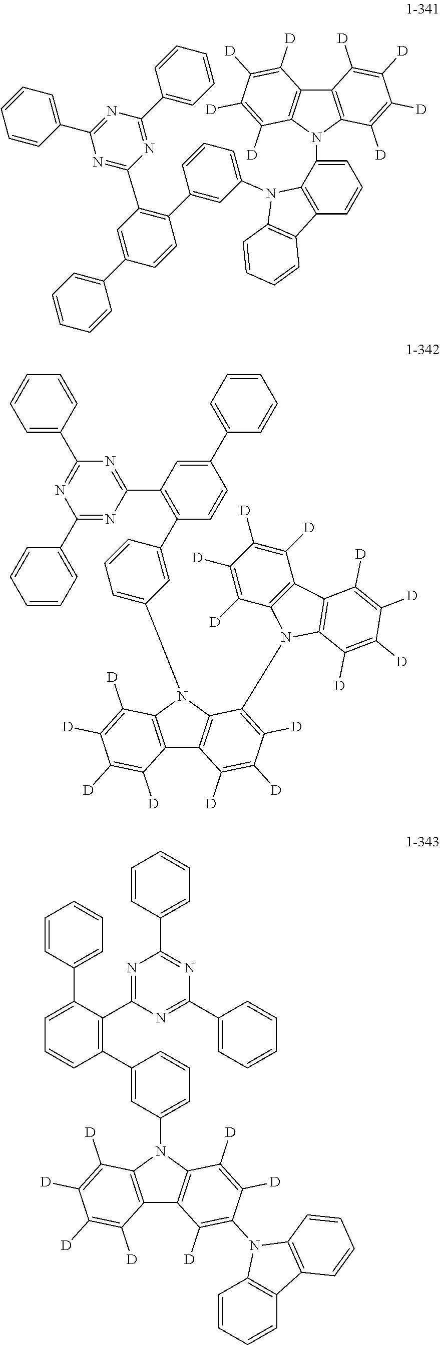 [SMILES: [2H]c1c([2H])c([2H])c2c(c1[2H])c1c([2H])c([2H])c([2H])c([2H])c1n2-c1cccc2c3ccccc3n(-c3cccc(-c4ccc(-c5ccccc5)cc4-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccc(-c3ccccc3)cc2-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cccc(-c2cccc(-c3ccccc3)c2-c2nc(-c3ccccc3)nc(-c3ccccc3)n2)c1]

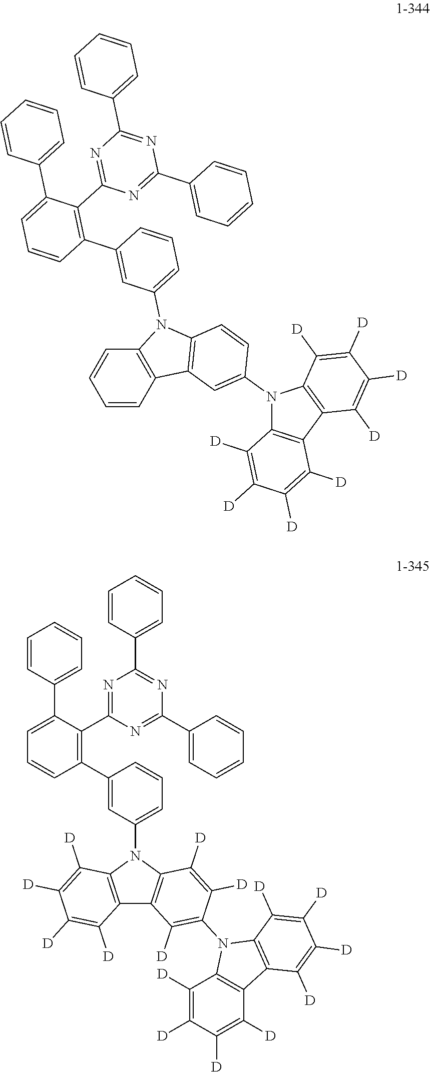 [SMILES: [2H]c1c([2H])c([2H])c2c(c1[2H])c1c([2H])c([2H])c([2H])c([2H])c1n2-c1ccc2c(c1)c1ccccc1n2-c1cccc(-c2cccc(-c3ccccc3)c2-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2cccc(-c3ccccc3)c2-c2nc(-c3ccccc3)nc(-c3ccccc3)n2)c1]

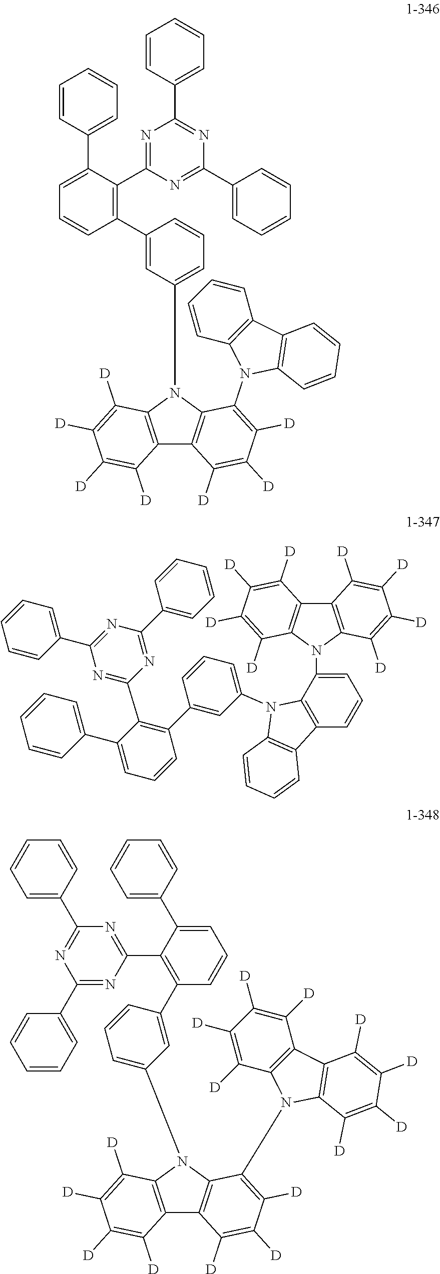 [SMILES: [2H]c1c([2H])c([2H])c2c(c1[2H])c1c([2H])c([2H])c([2H])c(-n3c4ccccc4c4ccccc43)c1n2-c1cccc(-c2cccc(-c3ccccc3)c2-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3cccc(-c4cccc(-c5ccccc5)c4-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2cccc(-c3ccccc3)c2-c2nc(-c3ccccc3)nc(-c3ccccc3)n2)c1]

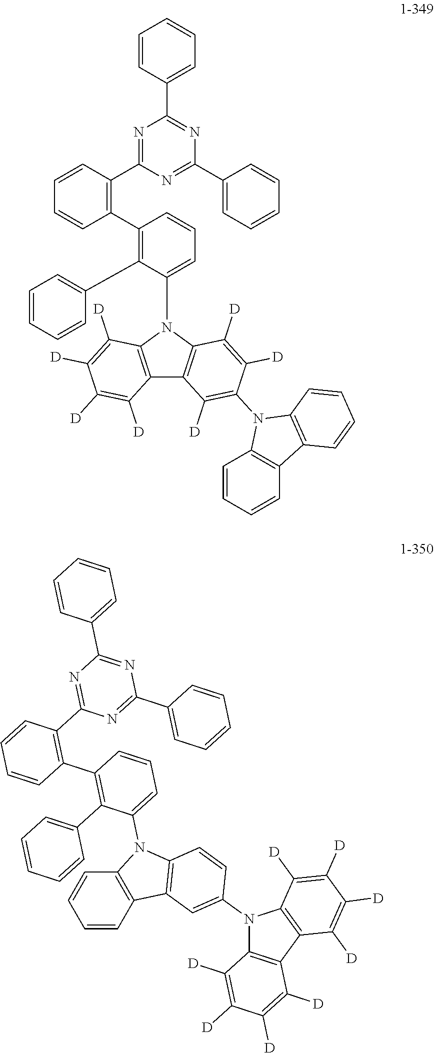 [SMILES: [2H]c1c([2H])c([2H])c2c(c1[2H])c1c([2H])c(-n3c4ccccc4c4ccccc43)c([2H])c([2H])c1n2-c1cccc(-c2ccccc2-c2nc(-c3ccccc3)nc(-c3ccccc3)n2)c1-c1ccccc1 ; [2H]c1c([2H])c([2H])c2c(c1[2H])c1c([2H])c([2H])c([2H])c([2H])c1n2-c1ccc2c(c1)c1ccccc1n2-c1cccc(-c2ccccc2-c2nc(-c3ccccc3)nc(-c3ccccc3)n2)c1-c1ccccc1]

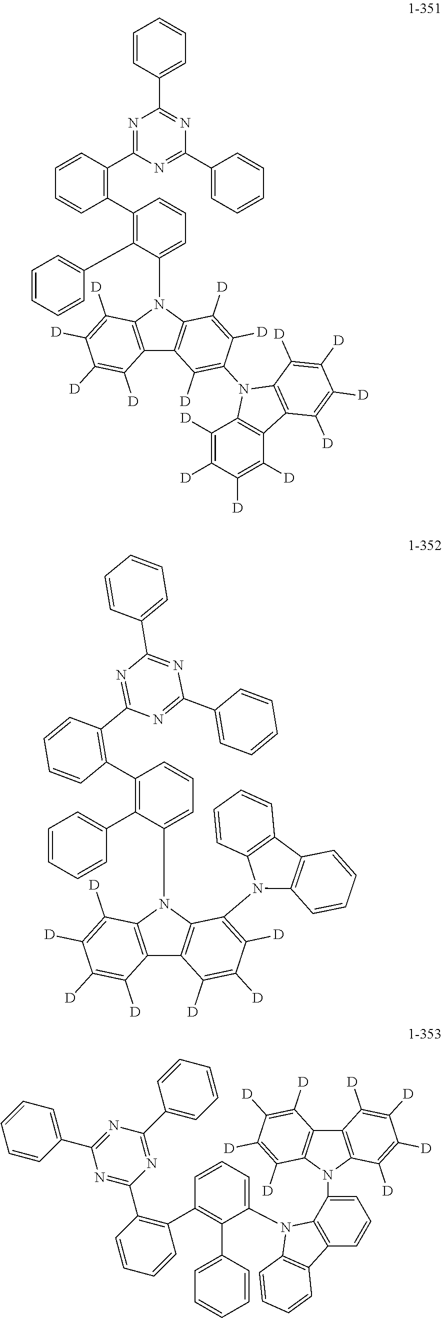 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccccc2-c2nc(-c3ccccc3)nc(-c3ccccc3)n2)c1-c1ccccc1 ; [2H]c1c([2H])c([2H])c2c(c1[2H])c1c([2H])c([2H])c([2H])c(-n3c4ccccc4c4ccccc43)c1n2-c1cccc(-c2ccccc2-c2nc(-c3ccccc3)nc(-c3ccccc3)n2)c1-c1ccccc1 ; [2H]c1c([2H])c([2H])c2c(c1[2H])c1c([2H])c([2H])c([2H])c([2H])c1n2-c1cccc2c3ccccc3n(-c3cccc(-c4ccccc4-c4nc(-c5ccccc5)nc(-c5ccccc5)n4)c3-c3ccccc3)c12]

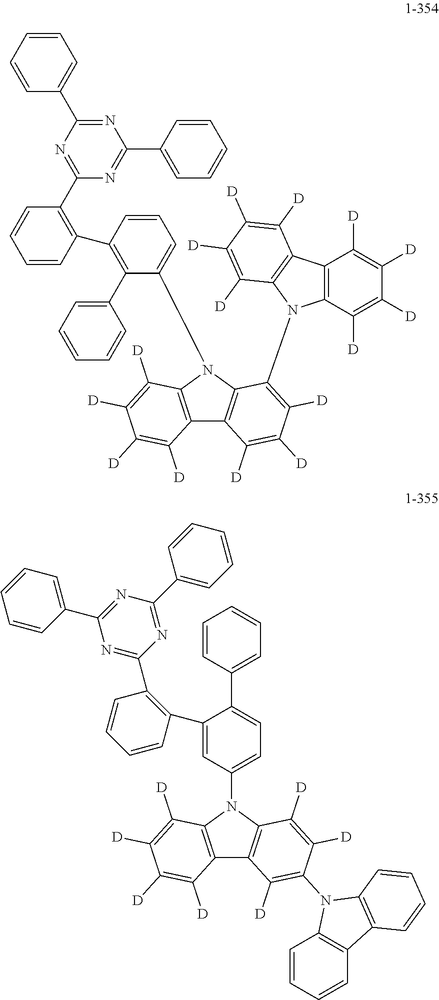 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccccc2-c2nc(-c3ccccc3)nc(-c3ccccc3)n2)c1-c1ccccc1 ; [2H]c1c([2H])c([2H])c2c(c1[2H])c1c([2H])c(-n3c4ccccc4c4ccccc43)c([2H])c([2H])c1n2-c1ccc(-c2ccccc2)c(-c2ccccc2-c2nc(-c3ccccc3)nc(-c3ccccc3)n2)c1]

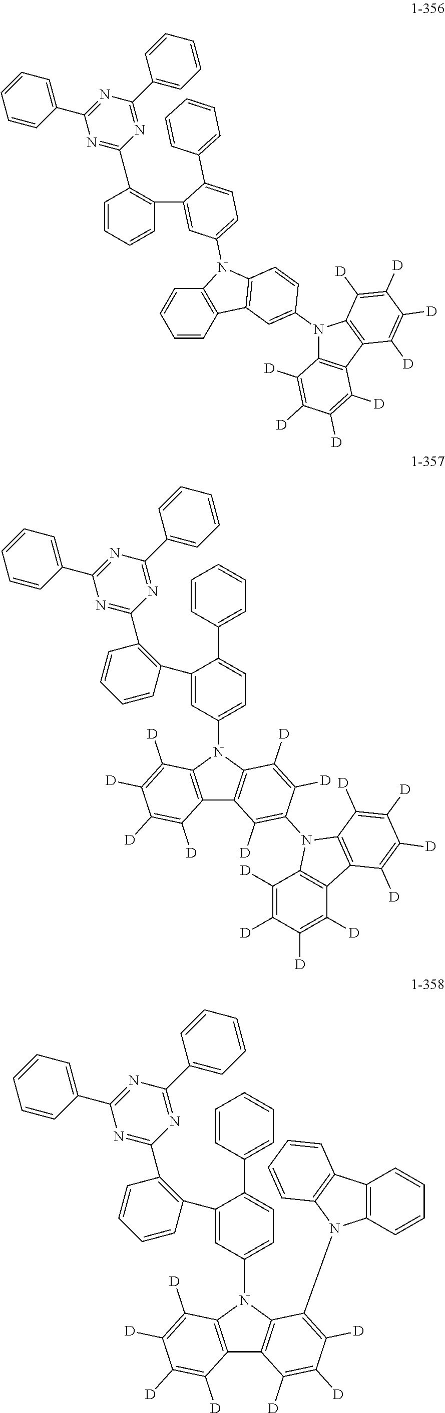 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2ccccc2)c(-c2ccccc2-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)c(-c2ccccc2-c2nc(-c3ccccc3)nc(-c3ccccc3)n2)c1 ; [2H]c1c([2H])c([2H])c2c(c1[2H])c1c([2H])c([2H])c([2H])c(-n3c4ccccc4c4ccccc43)c1n2-c1ccc(-c2ccccc2)c(-c2ccccc2-c2nc(-c3ccccc3)nc(-c3ccccc3)n2)c1]

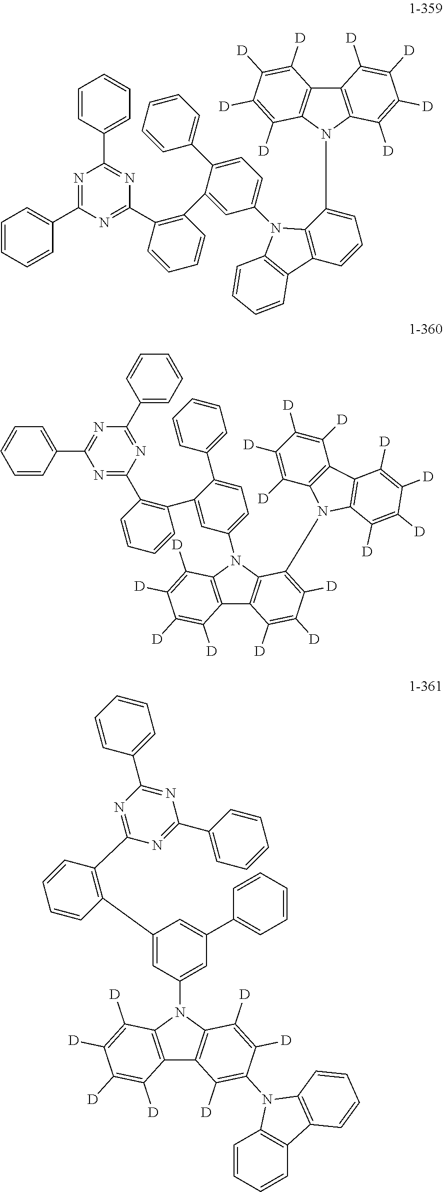 [SMILES: [2H]c1c([2H])c([2H])c2c(c1[2H])c1c([2H])c([2H])c([2H])c([2H])c1n2-c1cccc2c3ccccc3n(-c3ccc(-c4ccccc4)c(-c4ccccc4-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)c(-c2ccccc2-c2nc(-c3ccccc3)nc(-c3ccccc3)n2)c1 ; [2H]c1c([2H])c([2H])c2c(c1[2H])c1c([2H])c(-n3c4ccccc4c4ccccc43)c([2H])c([2H])c1n2-c1cc(-c2ccccc2)cc(-c2ccccc2-c2nc(-c3ccccc3)nc(-c3ccccc3)n2)c1]

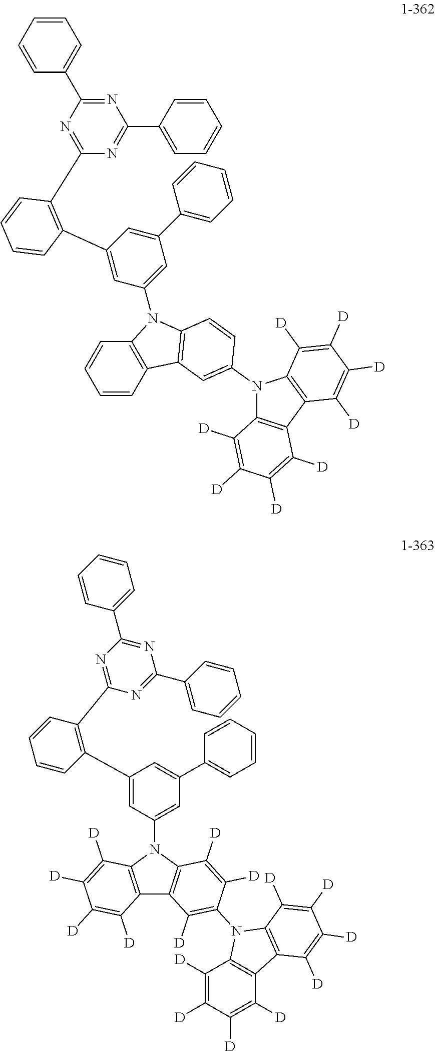 [SMILES: [2H]c1c([2H])c([2H])c2c(c1[2H])c1c([2H])c([2H])c([2H])c([2H])c1n2-c1ccc2c(c1)c1ccccc1n2-c1cc(-c2ccccc2)cc(-c2ccccc2-c2nc(-c3ccccc3)nc(-c3ccccc3)n2)c1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)cc(-c2ccccc2-c2nc(-c3ccccc3)nc(-c3ccccc3)n2)c1]

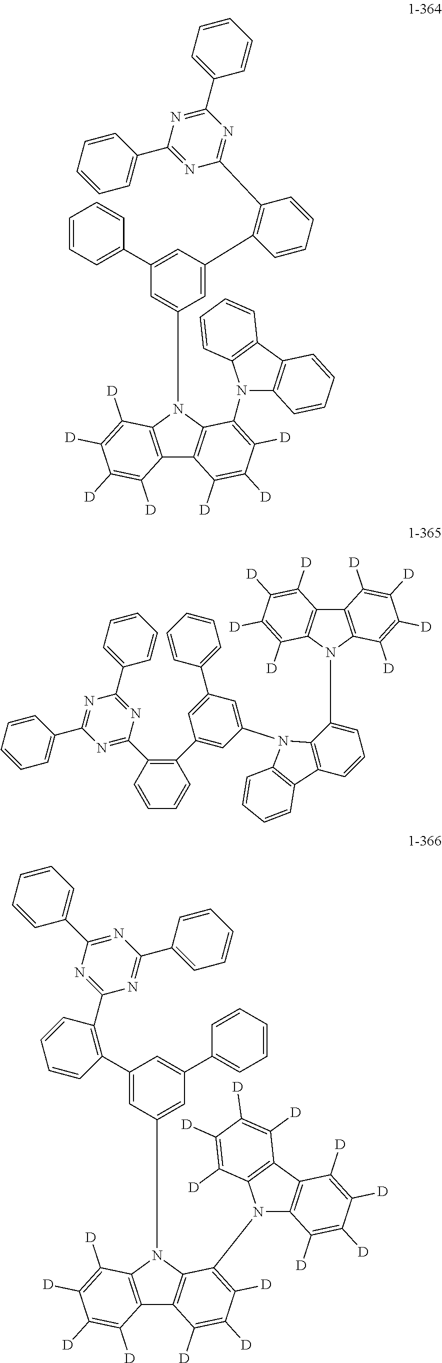 [SMILES: [2H]c1c([2H])c([2H])c2c(c1[2H])c1c([2H])c([2H])c([2H])c(-n3c4ccccc4c4ccccc43)c1n2-c1cc(-c2ccccc2)cc(-c2ccccc2-c2nc(-c3ccccc3)nc(-c3ccccc3)n2)c1 ; [2H]c1c([2H])c([2H])c2c(c1[2H])c1c([2H])c([2H])c([2H])c([2H])c1n2-c1cccc2c3ccccc3n(-c3cc(-c4ccccc4)cc(-c4ccccc4-c4nc(-c5ccccc5)nc(-c5ccccc5)n4)c3)c12 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2)cc(-c2ccccc2-c2nc(-c3ccccc3)nc(-c3ccccc3)n2)c1]

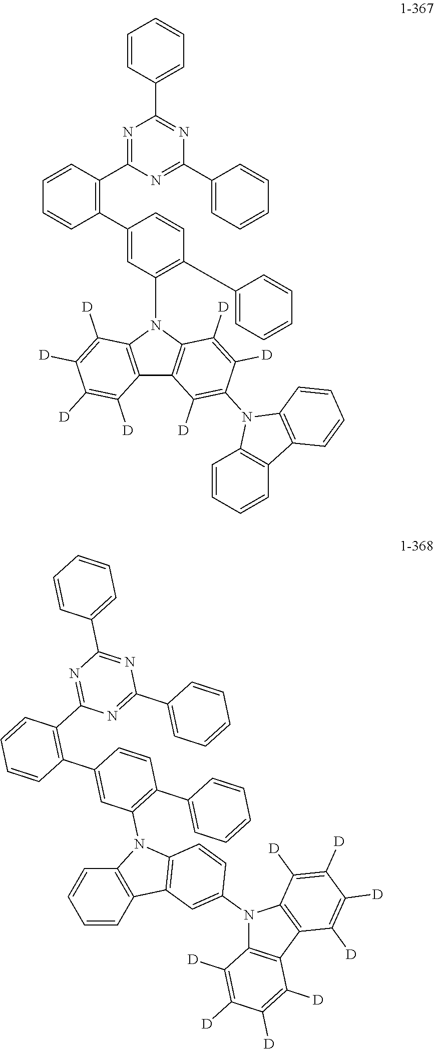 [SMILES: [2H]c1c([2H])c([2H])c2c(c1[2H])c1c([2H])c(-n3c4ccccc4c4ccccc43)c([2H])c([2H])c1n2-c1cc(-c2ccccc2-c2nc(-c3ccccc3)nc(-c3ccccc3)n2)ccc1-c1ccccc1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2-c2nc(-c3ccccc3)nc(-c3ccccc3)n2)ccc1-c1ccccc1]

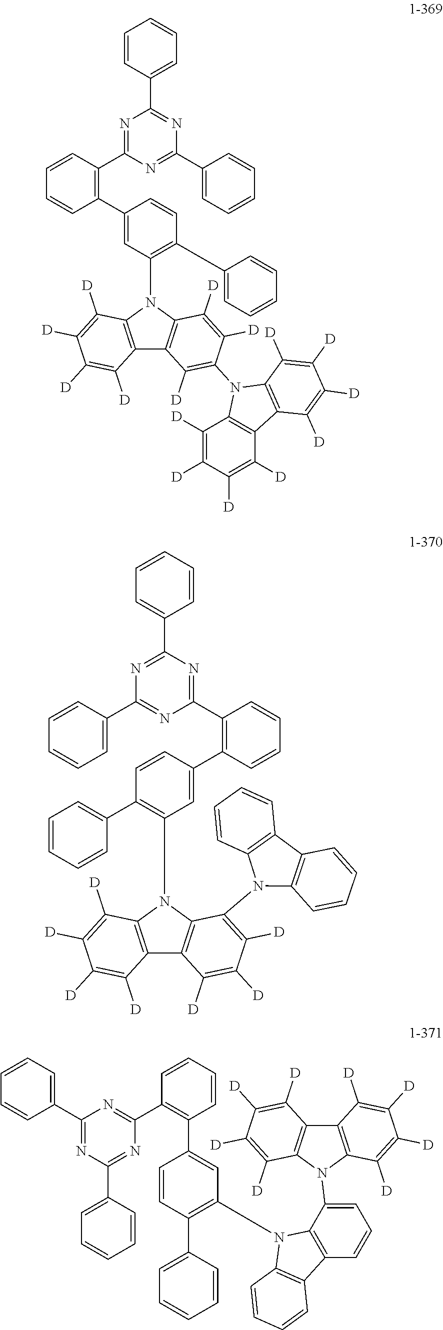 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2-c2nc(-c3ccccc3)nc(-c3ccccc3)n2)ccc1-c1ccccc1 ; [2H]c1c([2H])c([2H])c2c(c1[2H])c1c([2H])c([2H])c([2H])c(-n3c4ccccc4c4ccccc43)c1n2-c1cc(-c2ccccc2-c2nc(-c3ccccc3)nc(-c3ccccc3)n2)ccc1-c1ccccc1 ; [2H]c1c([2H])c([2H])c2c(c1[2H])c1c([2H])c([2H])c([2H])c([2H])c1n2-c1cccc2c3ccccc3n(-c3cc(-c4ccccc4-c4nc(-c5ccccc5)nc(-c5ccccc5)n4)ccc3-c3ccccc3)c12]

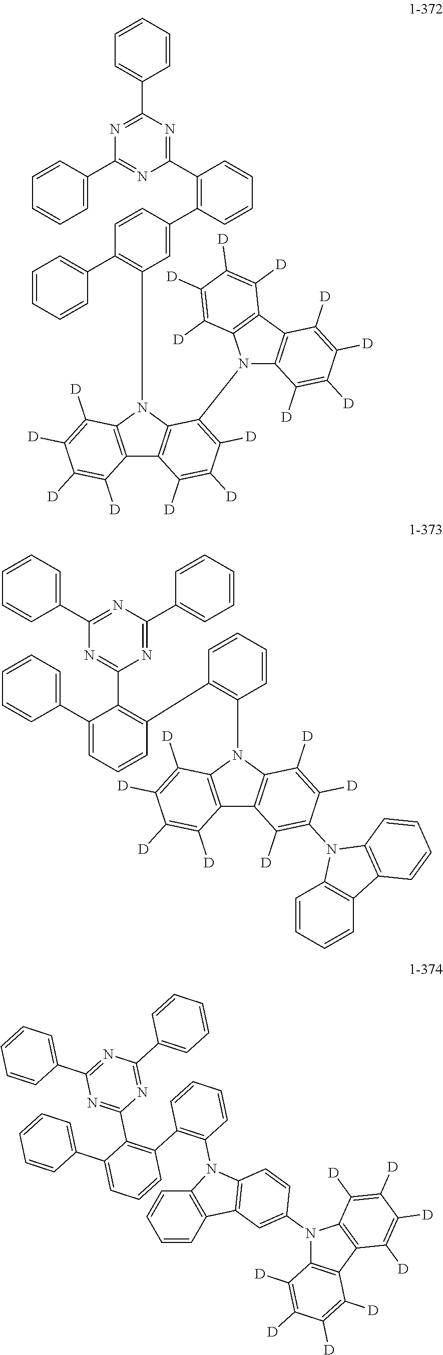 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cc(-c2ccccc2-c2nc(-c3ccccc3)nc(-c3ccccc3)n2)ccc1-c1ccccc1 ; [2H]c1c([2H])c([2H])c2c(c1[2H])c1c([2H])c(-n3c4ccccc4c4ccccc43)c([2H])c([2H])c1n2-c1ccccc1-c1cccc(-c2ccccc2)c1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1ccccc1-c1cccc(-c2ccccc2)c1-c1nc(-c2ccccc2)nc(-c2ccccc2)n1]

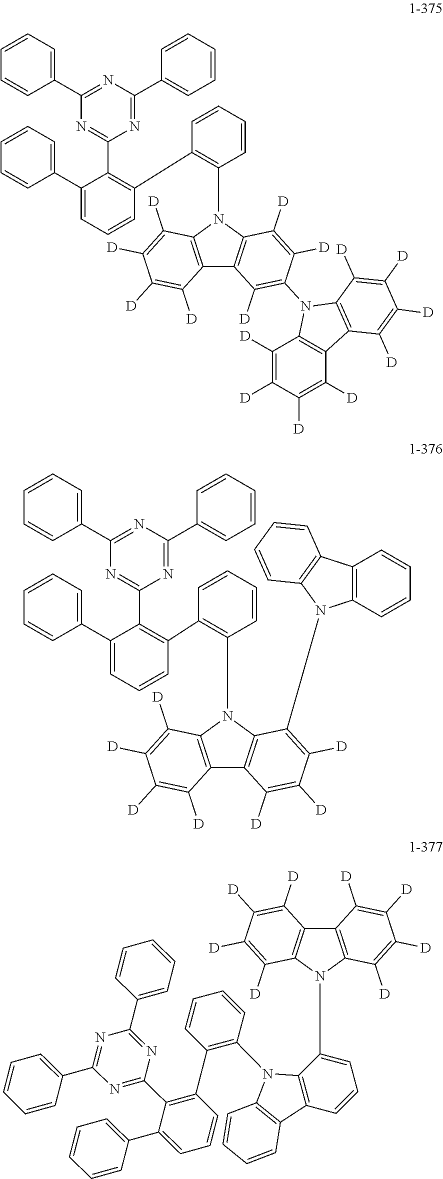 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1cccc(-c2ccccc2)c1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1ccccc1-c1cccc(-c2ccccc2)c1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3ccccc3-c3cccc(-c4ccccc4)c3-c3nc(-c4ccccc4)nc(-c4ccccc4)n3)c12]

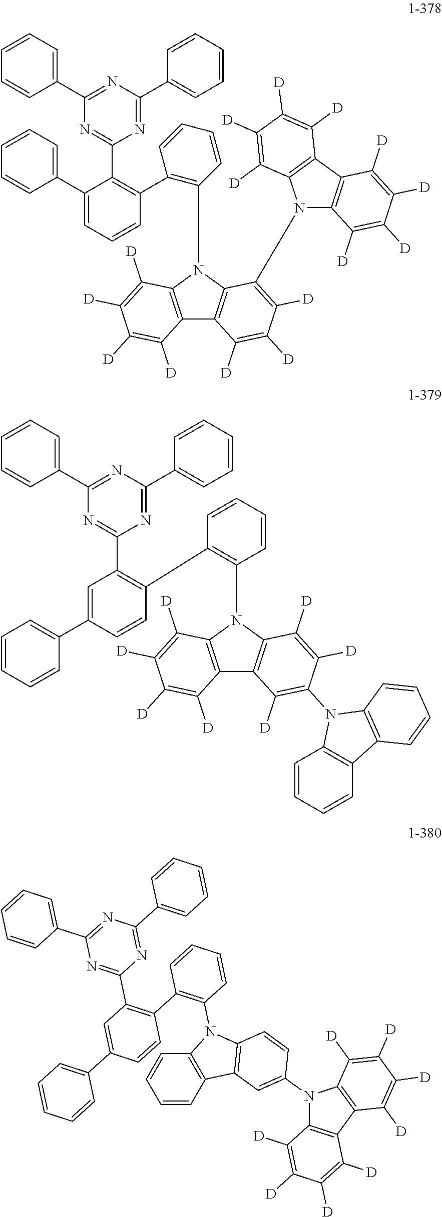 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1cccc(-c2ccccc2)c1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1ccccc1-c1ccc(-c2ccccc2)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1ccccc1-c1ccc(-c2ccccc2)cc1-c1nc(-c2ccccc2)nc(-c2ccccc2)n1]

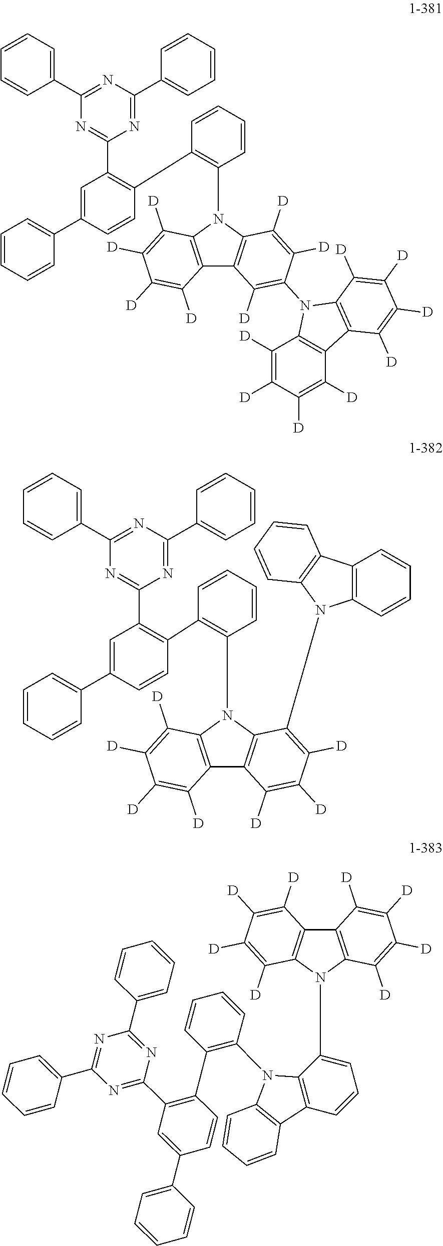 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1ccc(-c2ccccc2)cc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1ccccc1-c1ccc(-c2ccccc2)cc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3ccccc3-c3ccc(-c4ccccc4)cc3-c3nc(-c4ccccc4)nc(-c4ccccc4)n3)c12]

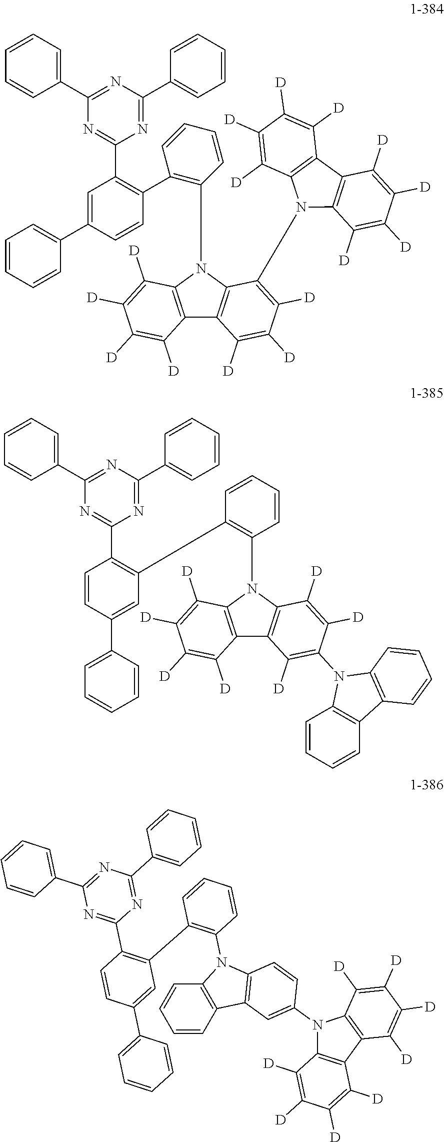 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1ccc(-c2ccccc2)cc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1ccccc1-c1cc(-c2ccccc2)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1ccccc1-c1cc(-c2ccccc2)ccc1-c1nc(-c2ccccc2)nc(-c2ccccc2)n1]

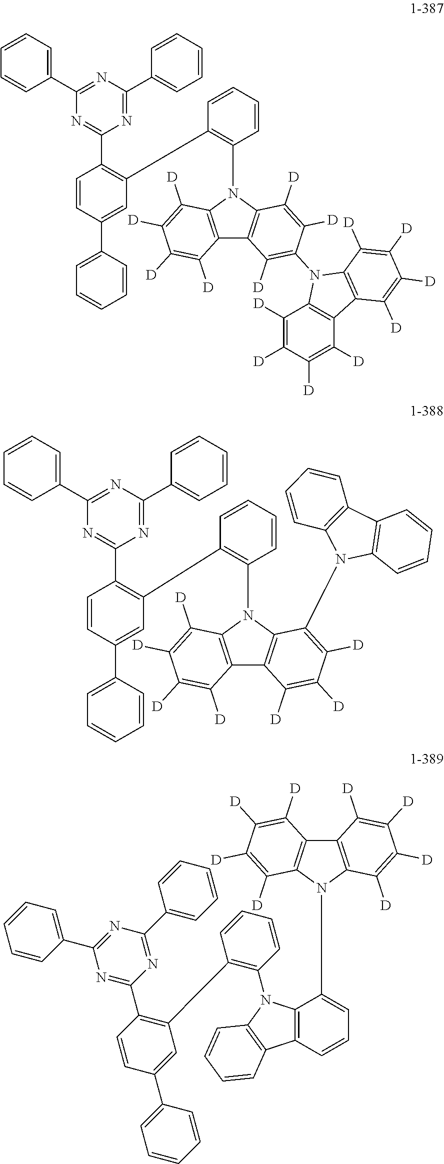 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1cc(-c2ccccc2)ccc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1ccccc1-c1cc(-c2ccccc2)ccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3ccccc3-c3cc(-c4ccccc4)ccc3-c3nc(-c4ccccc4)nc(-c4ccccc4)n3)c12]

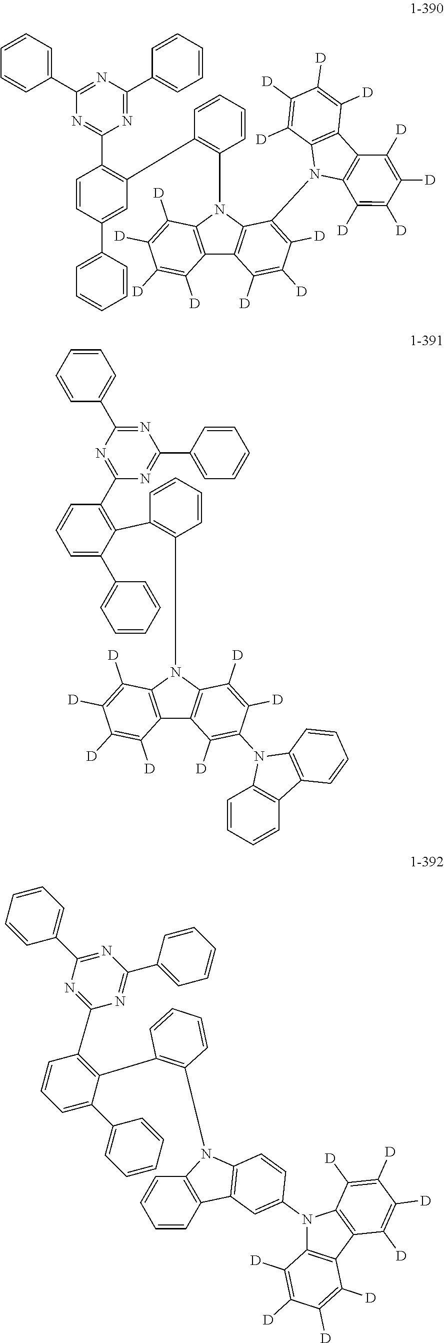

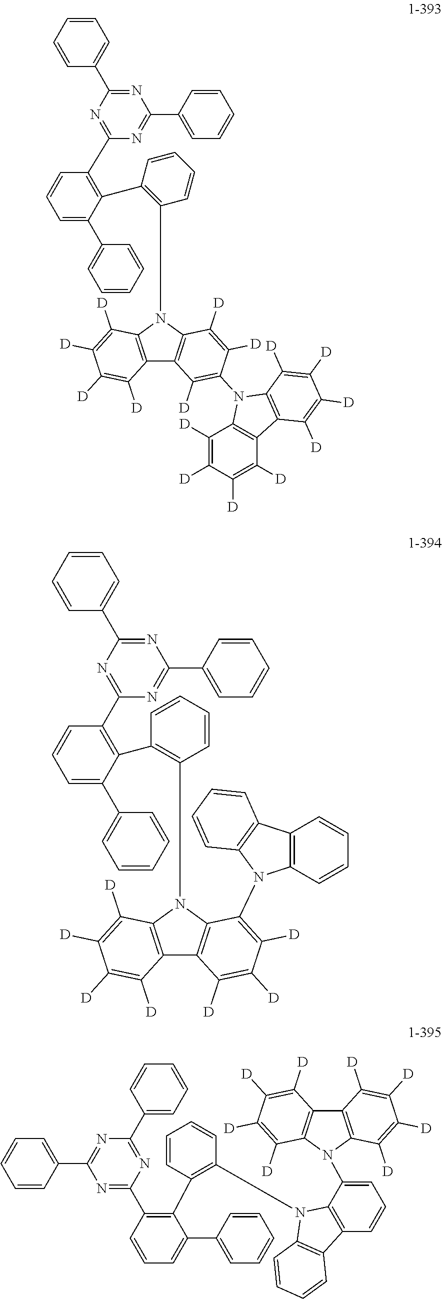 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccccc1-c1c(-c2ccccc2)cccc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1ccccc1-c1c(-c2ccccc2)cccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3ccccc3-c3c(-c4ccccc4)cccc3-c3nc(-c4ccccc4)nc(-c4ccccc4)n3)c12]

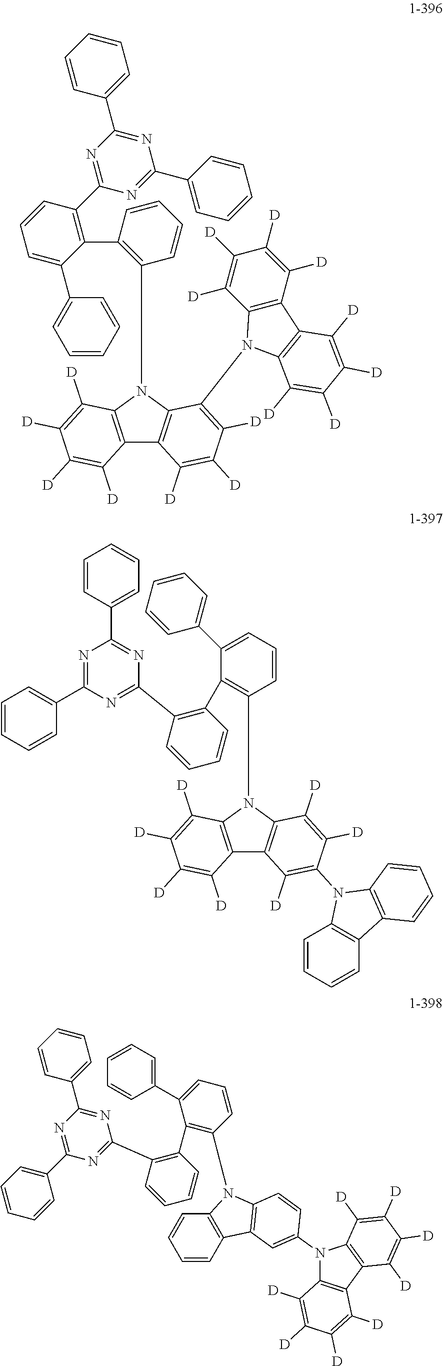 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccccc1-c1c(-c2ccccc2)cccc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1cccc(-c2ccccc2)c1-c1ccccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1cccc(-c2ccccc2)c1-c1ccccc1-c1nc(-c2ccccc2)nc(-c2ccccc2)n1]

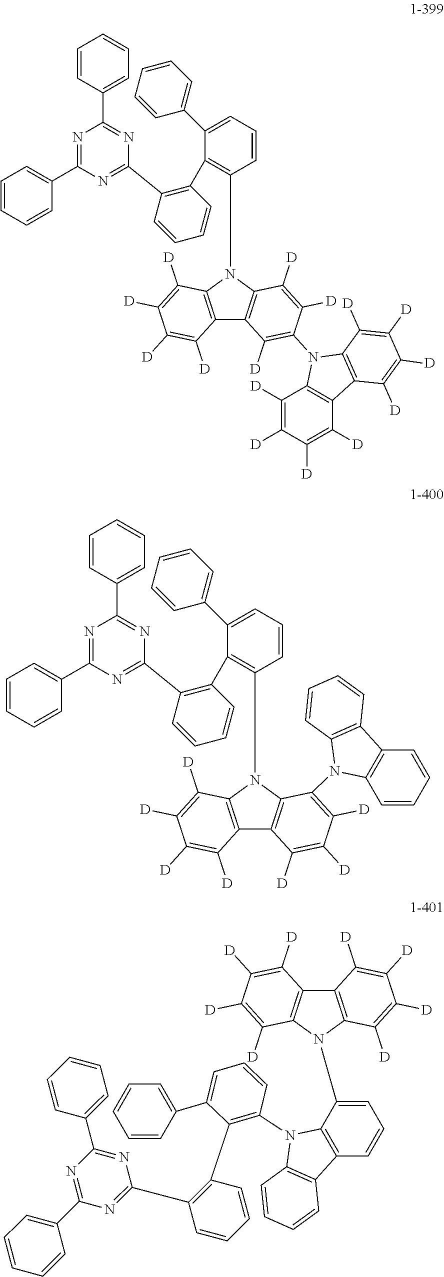 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc(-c2ccccc2)c1-c1ccccc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1cccc(-c2ccccc2)c1-c1ccccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3cccc(-c4ccccc4)c3-c3ccccc3-c3nc(-c4ccccc4)nc(-c4ccccc4)n3)c12]

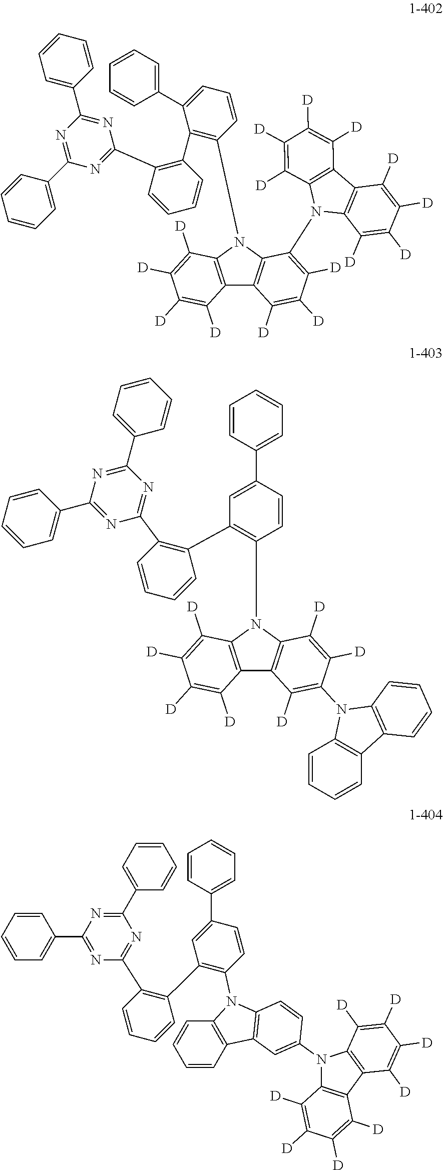 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1cccc(-c2ccccc2)c1-c1ccccc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1ccc(-c2ccccc2)cc1-c1ccccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1ccc(-c2ccccc2)cc1-c1ccccc1-c1nc(-c2ccccc2)nc(-c2ccccc2)n1]

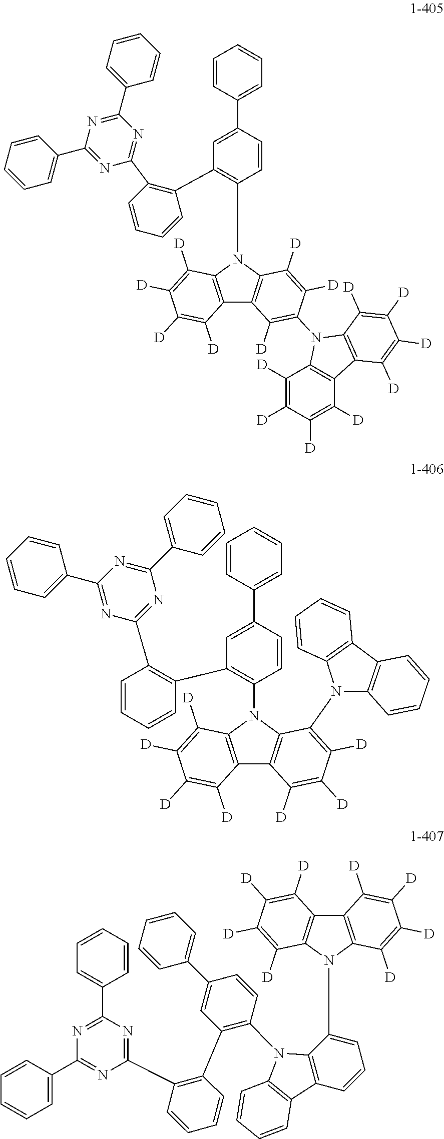 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccccc2)cc1-c1ccccc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1ccc(-c2ccccc2)cc1-c1ccccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3ccc(-c4ccccc4)cc3-c3ccccc3-c3nc(-c4ccccc4)nc(-c4ccccc4)n3)c12]

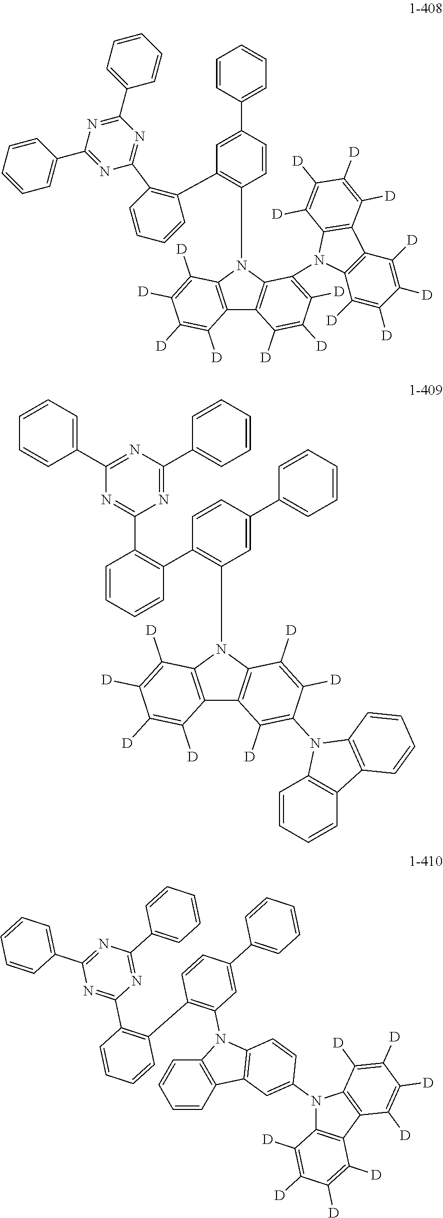 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccccc2)cc1-c1ccccc1-c1nc(-c2ccccc2)nc(-c2ccccc2)n1 ; [2H]c1c([2H])c([2H])c2c(c1[2H])c1c([2H])c(-n3c4ccccc4c4ccccc43)c([2H])c([2H])c1n2-c1cc(-c2ccccc2)ccc1-c1ccccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1ccc2c(c1)c1ccccc1n2-c1cc(-c2ccccc2)ccc1-c1ccccc1-c1nc(-c2ccccc2)nc(-c2ccccc2)n1]

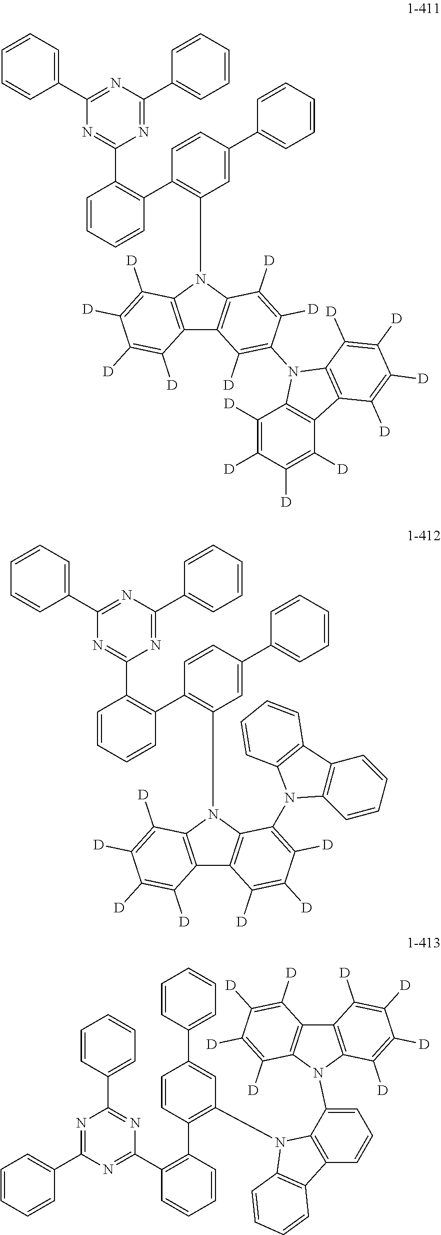 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccccc2)ccc1-c1ccccc1-c1nc(-c2ccccc2)nc(-c2ccccc2)n1 ; [2H]c1c([2H])c([2H])c2c(c1[2H])c1c([2H])c([2H])c([2H])c(-n3c4ccccc4c4ccccc43)c1n2-c1cc(-c2ccccc2)ccc1-c1ccccc1-c1nc(-c2ccccc2)nc(-c2ccccc2)n1 ; [2H]c1c([2H])c([2H])c2c(c1[2H])c1c([2H])c([2H])c([2H])c([2H])c1n2-c1cccc2c3ccccc3n(-c3cc(-c4ccccc4)ccc3-c3ccccc3-c3nc(-c4ccccc4)nc(-c4ccccc4)n3)c12]

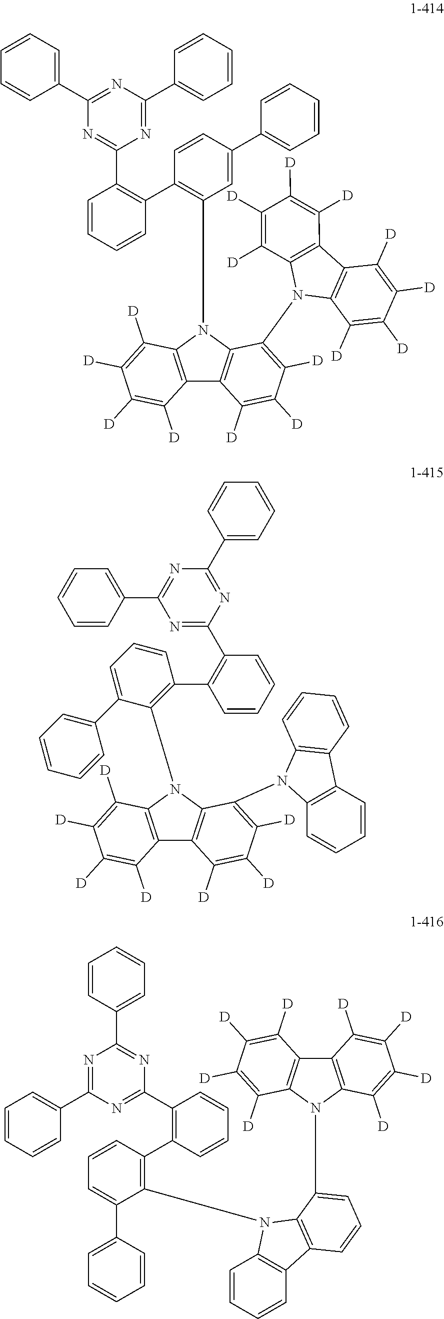

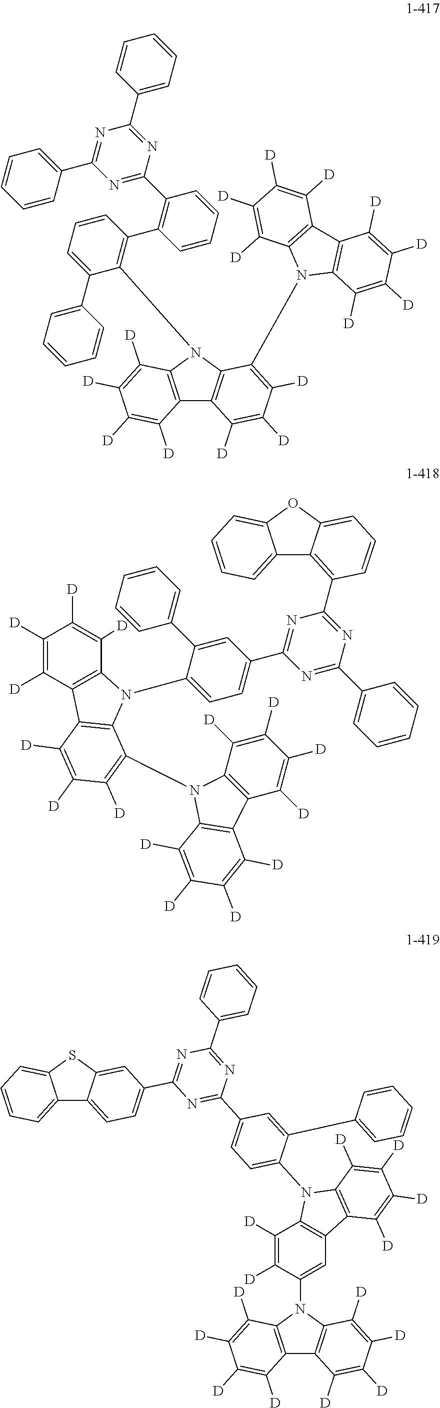

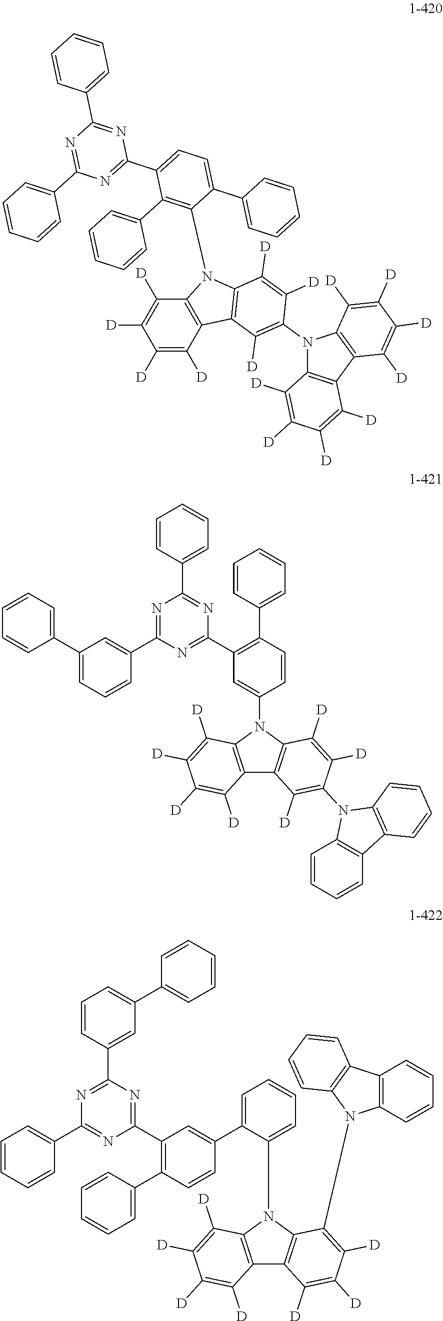 [SMILES: [2H]c1c([2H])c([2H])c2c(c1[2H])c1c([2H])c([2H])c([2H])c([2H])c1n2-c1c([2H])c([2H])c2c(c1[2H])c1c([2H])c([2H])c([2H])c([2H])c1n2-c1c(-c2ccccc2)ccc(-c2nc(-c3ccccc3)nc(-c3ccccc3)n2)c1-c1ccccc1 ; [2H]c1c([2H])c([2H])c2c(c1[2H])c1c([2H])c(-n3c4ccccc4c4ccccc43)c([2H])c([2H])c1n2-c1ccc(-c2ccccc2)c(-c2nc(-c3ccccc3)nc(-c3cccc(-c4ccccc4)c3)n2)c1 ; [2H]c1c([2H])c([2H])c2c(c1[2H])c1c([2H])c([2H])c([2H])c(-n3c4ccccc4c4ccccc43)c1n2-c1ccccc1-c1ccc(-c2ccccc2)c(-c2nc(-c3ccccc3)nc(-c3cccc(-c4ccccc4)c3)n2)c1]

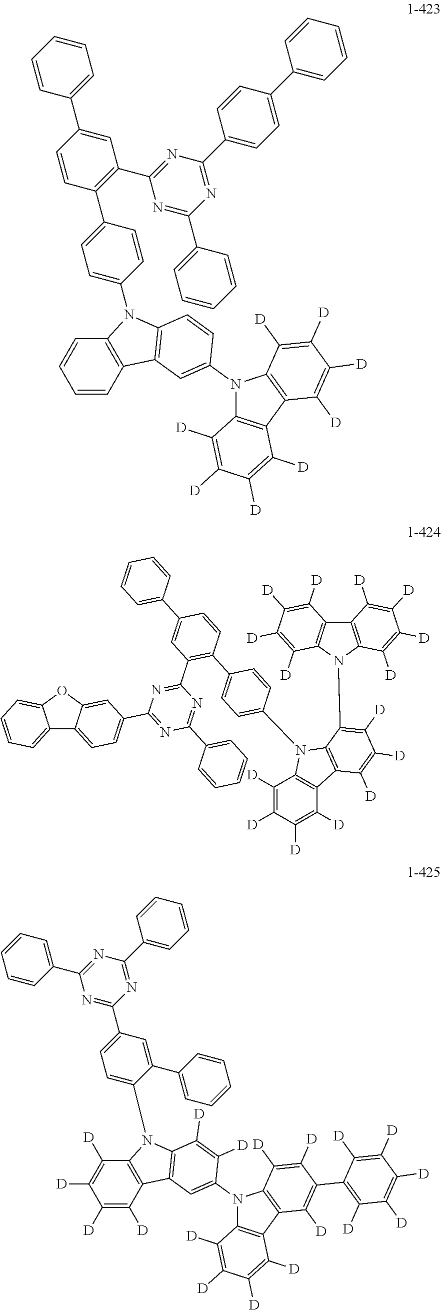

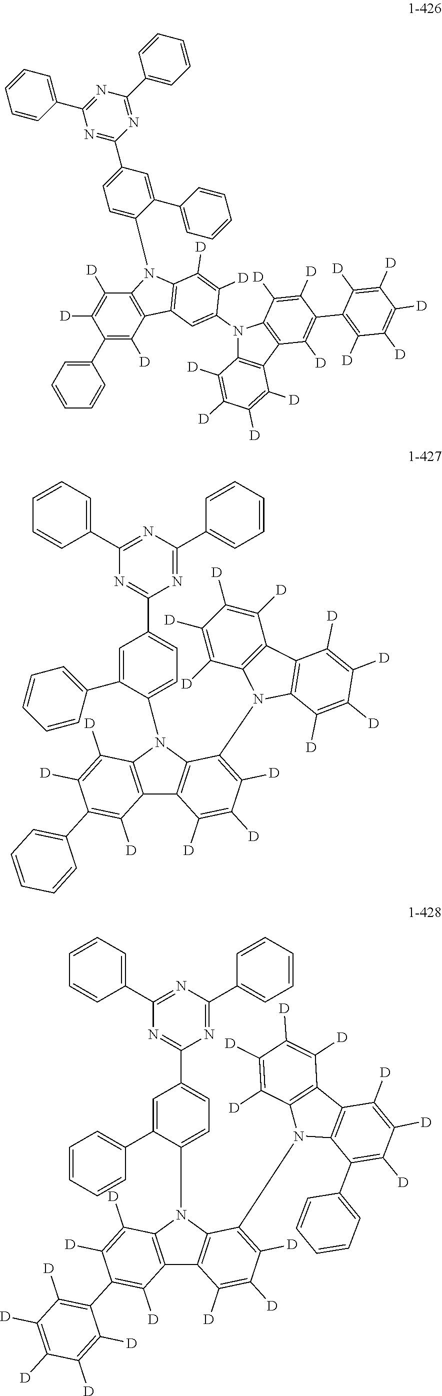

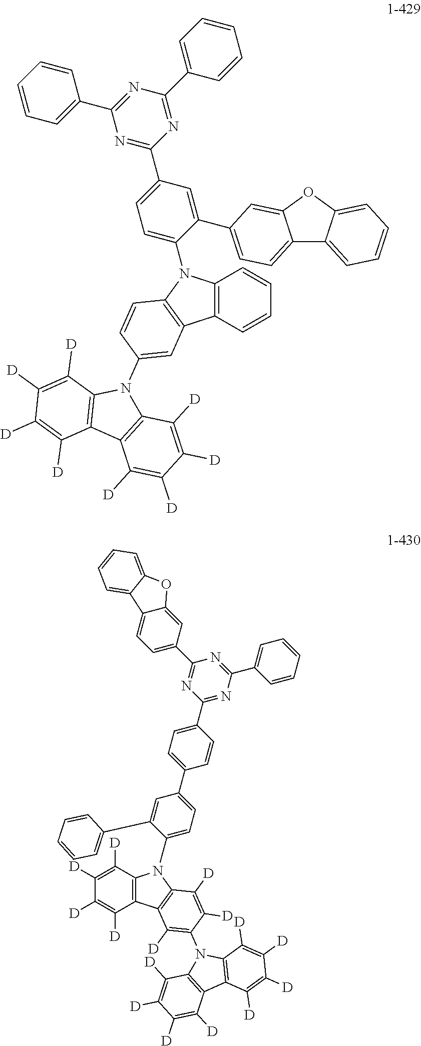 [SMILES: [2H]c1c([2H])c([2H])c2c(c1[2H])c1c([2H])c([2H])c([2H])c([2H])c1n2-c1ccc2c(c1)c1ccccc1n2-c1ccc(-c2nc(-c3ccccc3)nc(-c3ccccc3)n2)cc1-c1ccc2c(c1)oc1ccccc12 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccc5c(c4)oc4ccccc45)n3)cc2)cc1-c1ccccc1]

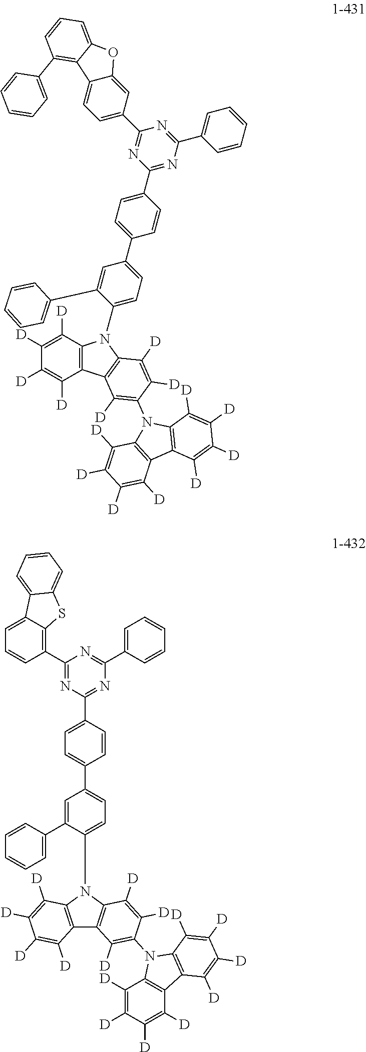 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccc5c(c4)oc4cccc(-c6ccccc6)c45)n3)cc2)cc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4cccc5c4sc4ccccc45)n3)cc2)cc1-c1ccccc1]

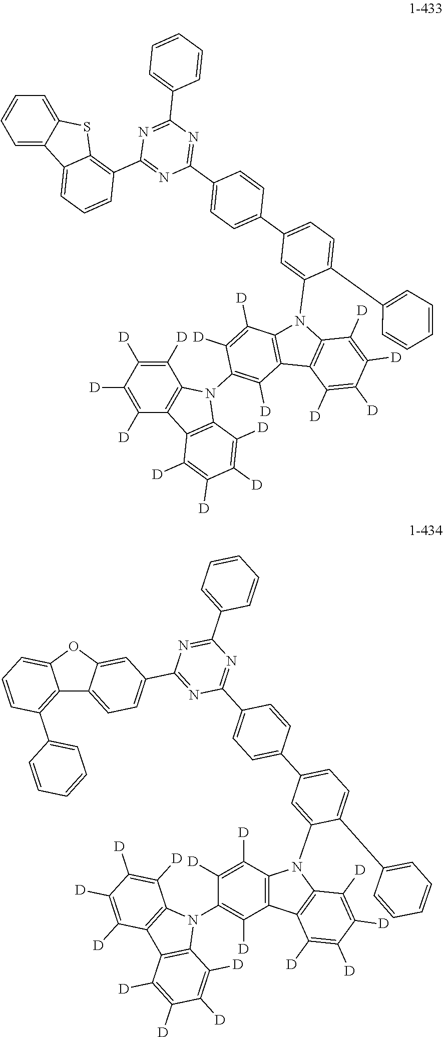 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4cccc5c4sc4ccccc45)n3)cc2)ccc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4ccc5c(c4)oc4cccc(-c6ccccc6)c45)n3)cc2)ccc1-c1ccccc1]

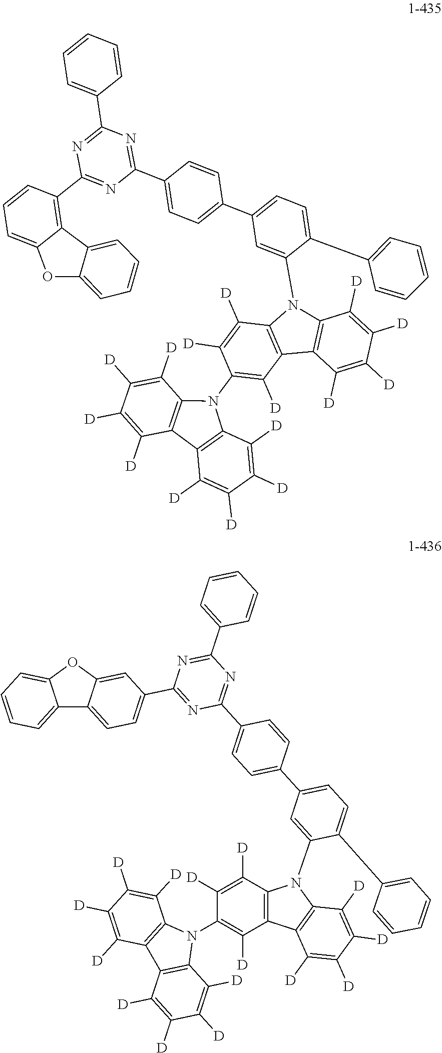 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4cccc5oc6ccccc6c45)n3)cc2)ccc1-c1ccccc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2ccc(-c3nc(-c4ccccc4)nc(-c4ccc5c(c4)oc4ccccc45)n3)cc2)ccc1-c1ccccc1]

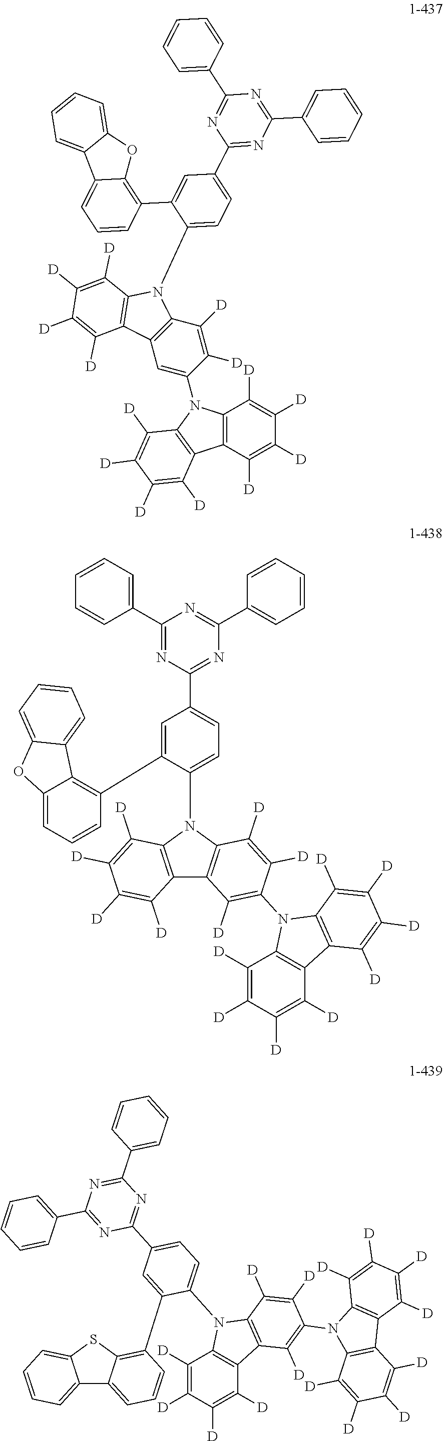

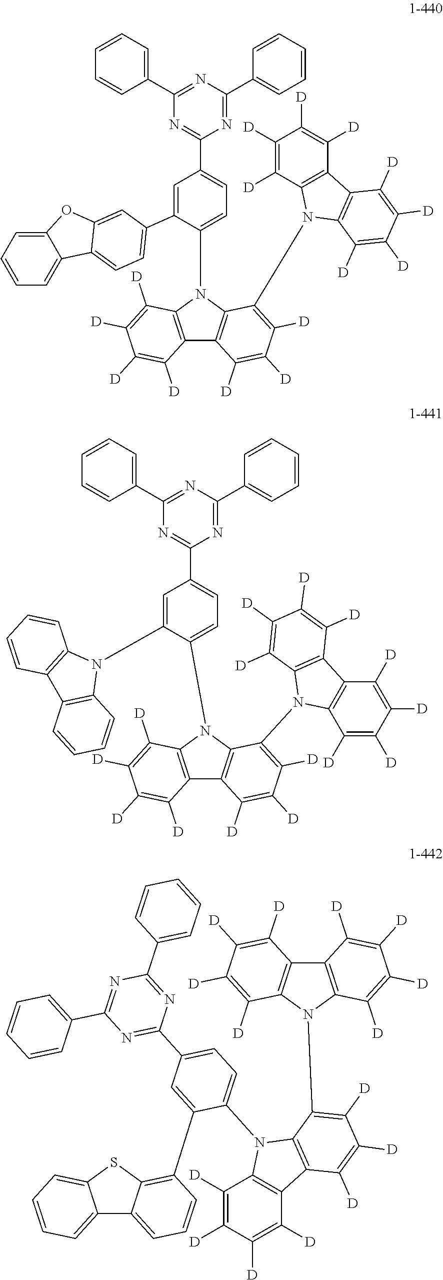

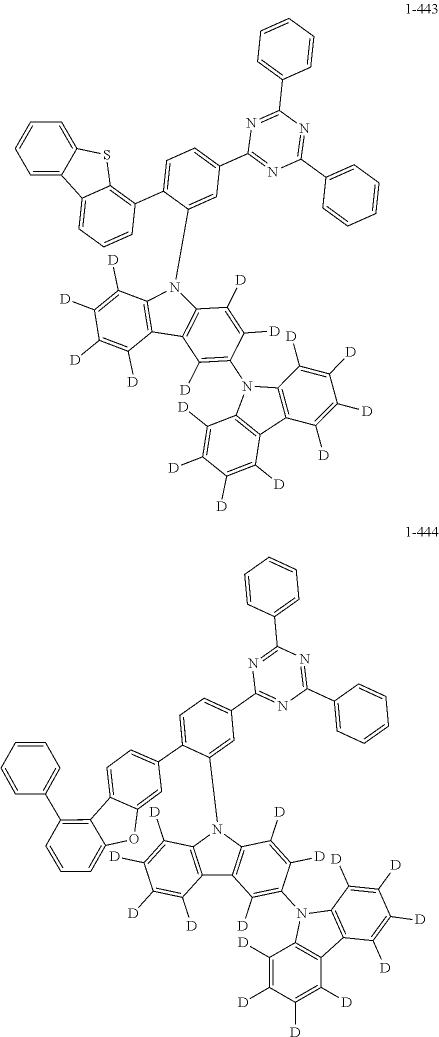 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2nc(-c3ccccc3)nc(-c3ccccc3)n2)ccc1-c1cccc2c1sc1ccccc12 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2nc(-c3ccccc3)nc(-c3ccccc3)n2)ccc1-c1ccc2c(c1)oc1cccc(-c3ccccc3)c12]

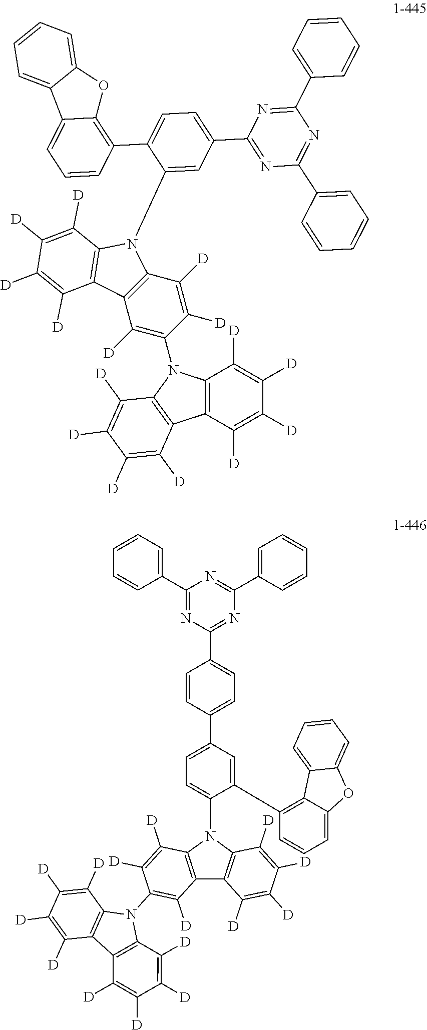 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cc(-c2nc(-c3ccccc3)nc(-c3ccccc3)n2)ccc1-c1cccc2c1oc1ccccc12 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)cc1-c1cccc2oc3ccccc3c12]

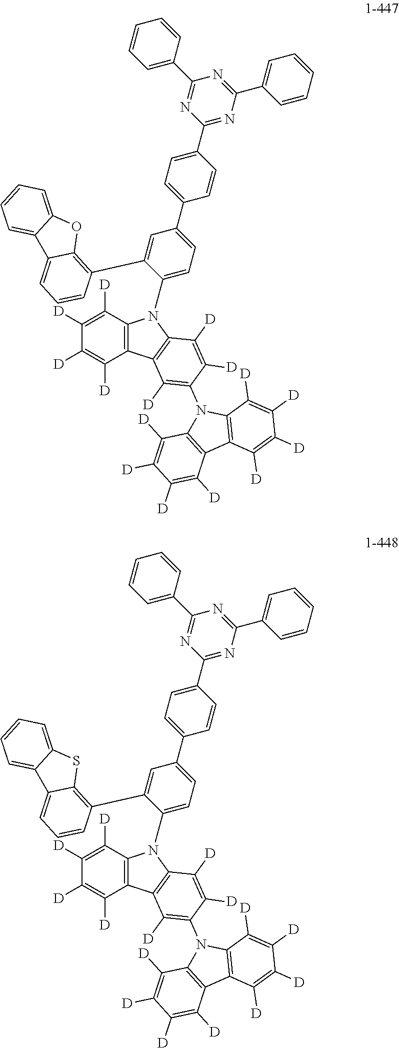 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)cc1-c1cccc2c1oc1ccccc12 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2)cc1-c1cccc2c1sc1ccccc12]

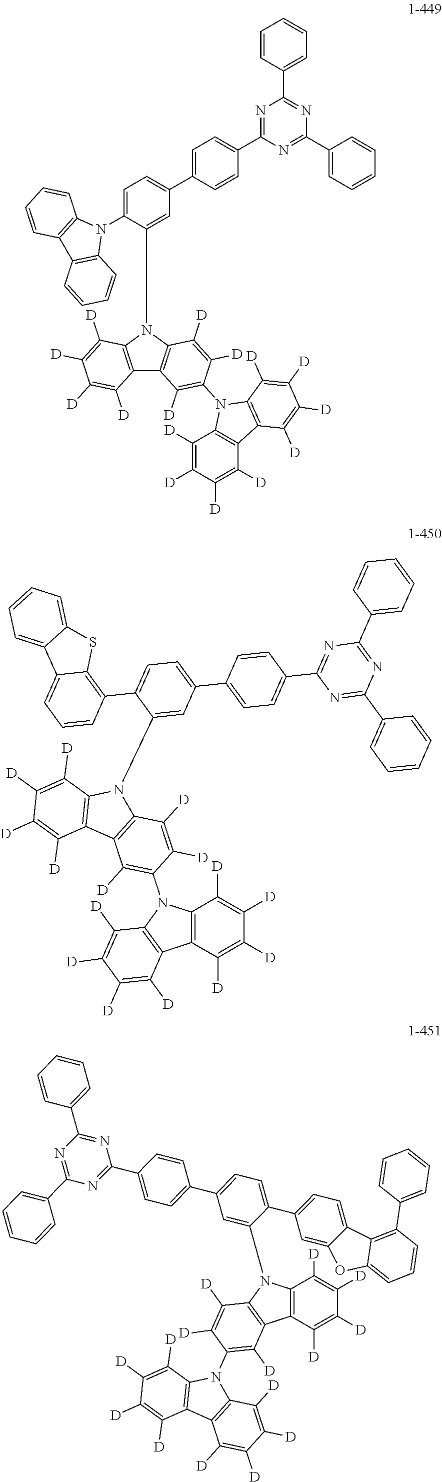

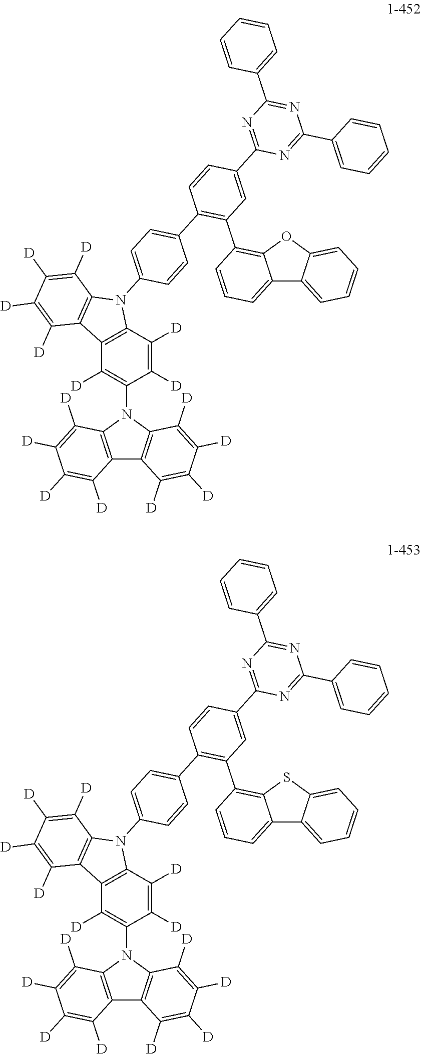 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2-c2cccc3c2oc2ccccc23)cc1 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2-c2cccc3c2sc2ccccc23)cc1]

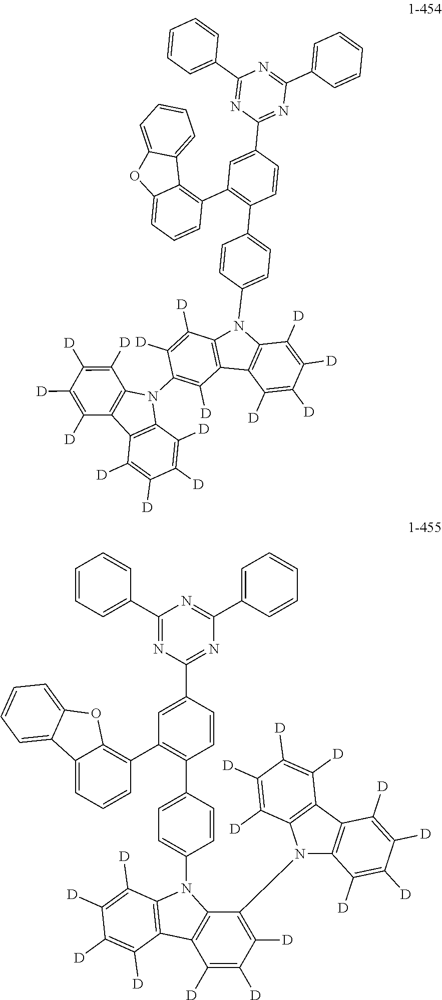 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc(-c2ccc(-c3nc(-c4ccccc4)nc(-c4ccccc4)n3)cc2-c2cccc3oc4ccccc4c23)cc1 ; [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)cc2-c2cccc3c2oc2ccccc23)cc1]

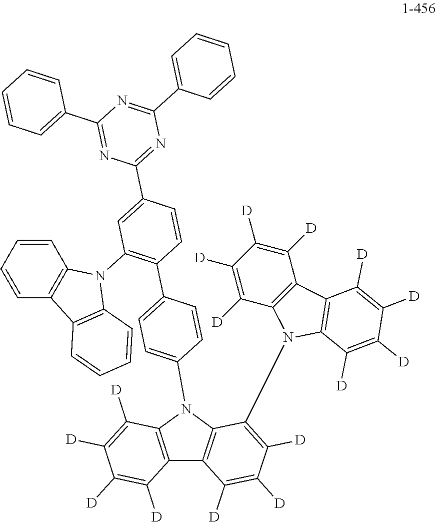 [SMILES: [2H]c1c([2H])c([2H])c2c(c1[2H])c1c([2H])c([2H])c([2H])c(-n3c4c([2H])c([2H])c([2H])c([2H])c4c4c([2H])c([2H])c([2H])c([2H])c43)c1n2-c1ccc(-c2ccc(-c3nc(-c4ccccc4)nc(-c4ccccc4)n3)cc2-n2c3ccccc3c3ccccc32)cc1]

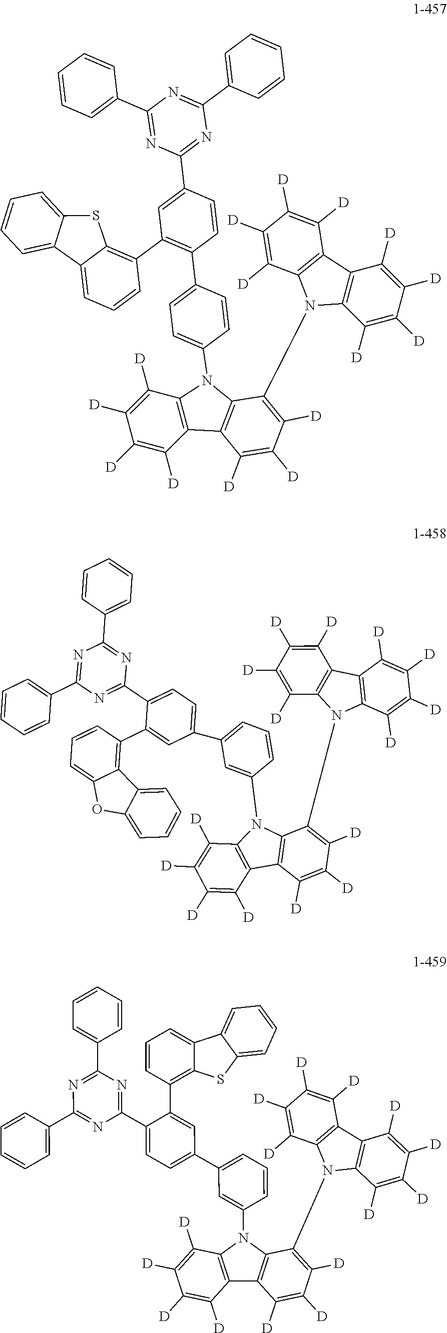

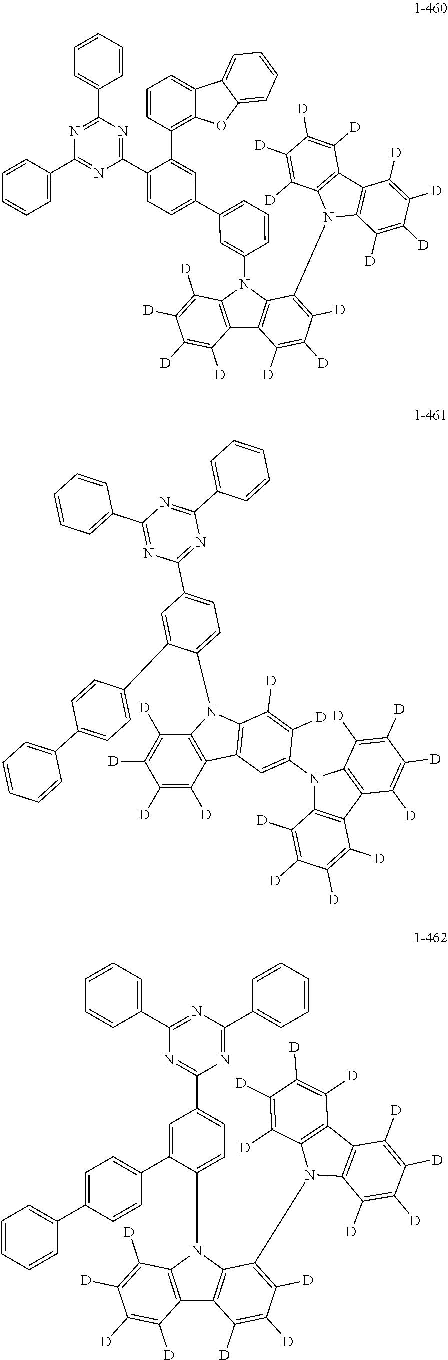

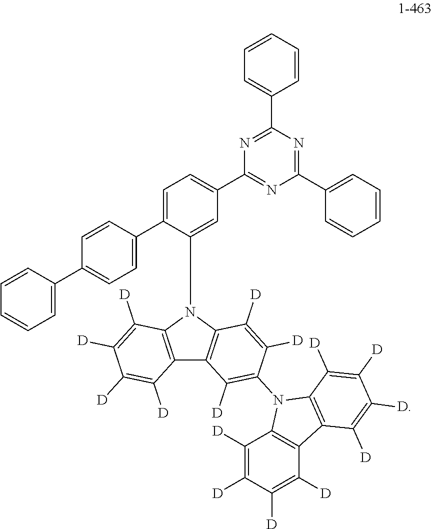

7. An organic light-emitting device comprising: a first electrode; a second electrode provided to face the first electrode; and one or more organic material layers provided between the first electrode and the second electrode, wherein at least one layer of the one or more organic material layers comprises the heterocyclic compound according to claim 1.

8. The organic light-emitting device according to claim 7, wherein the at least one layer of the one or more organic material layers comprises a light-emitting layer, and the light-emitting layer comprises the heterocyclic compound.

9. The organic light-emitting device according to claim 8, wherein the light-emitting layer comprises a host material, and the host material comprises the heterocyclic compound.

10. The organic light-emitting device according to claim 9, wherein the host material further comprises a heterocyclic compound represented by the following Chemical Formula 3 or Chemical Formula 4: 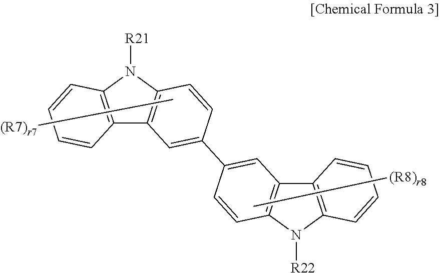
wherein, in Chemical Formula 3,
R21 and R22 are the same as or different from each other, and are each independently a substituted or unsubstituted C6 to C60 aryl group; or a substituted or unsubstituted C2 to C60 heteroaryl group;
R7 and R8 are the same as or different from each other, and are each independently hydrogen, deuterium, a halogen group, a cyano group, a substituted or unsubstituted C1 to C60 alkyl group; a substituted or unsubstituted C6 to C60 aryl group; or a substituted or unsubstituted C2 to C60 heteroaryl group; and
r7 and r8 are each an integer from 0 to 7, and when r7 is 2 or greater, R7 are the same as or different from each other, and when r8 is 2 or greater, R8 are the same as or different from each other; and 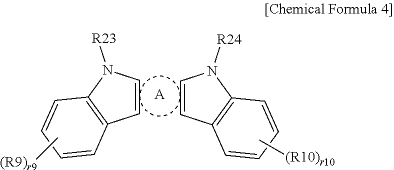
wherein, in Chemical Formula 4,
A is a substituted or unsubstituted C6 to C60 aromatic hydrocarbon ring;
R23 and R24 are the same as or different from each other, and are each independently a substituted or unsubstituted C6 to C60 aryl group; or a substituted or unsubstituted C2 to C60 heteroaryl group;
R9 and R10 are the same as or different from each other, and are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group; and
r9 and r10 are each an integer from 0 to 4, and when r9 is 2 or greater, R9 are the same as or different from each other, and when r10 is 2 or greater, R10 are the same as or different from each other.

11. The organic light-emitting device according to claim 10, wherein Chemical Formula 3 is represented by any one of the following compounds:

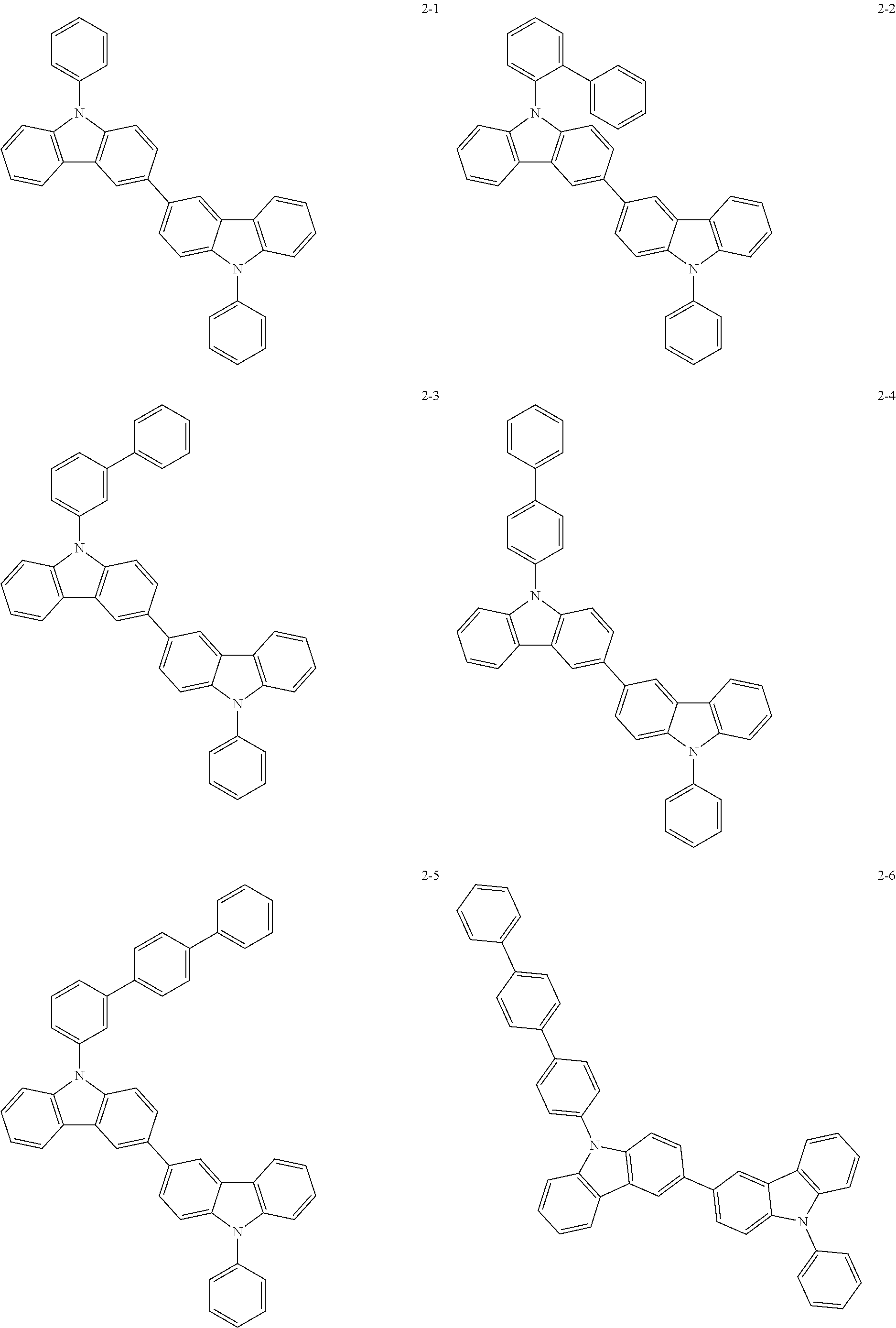 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

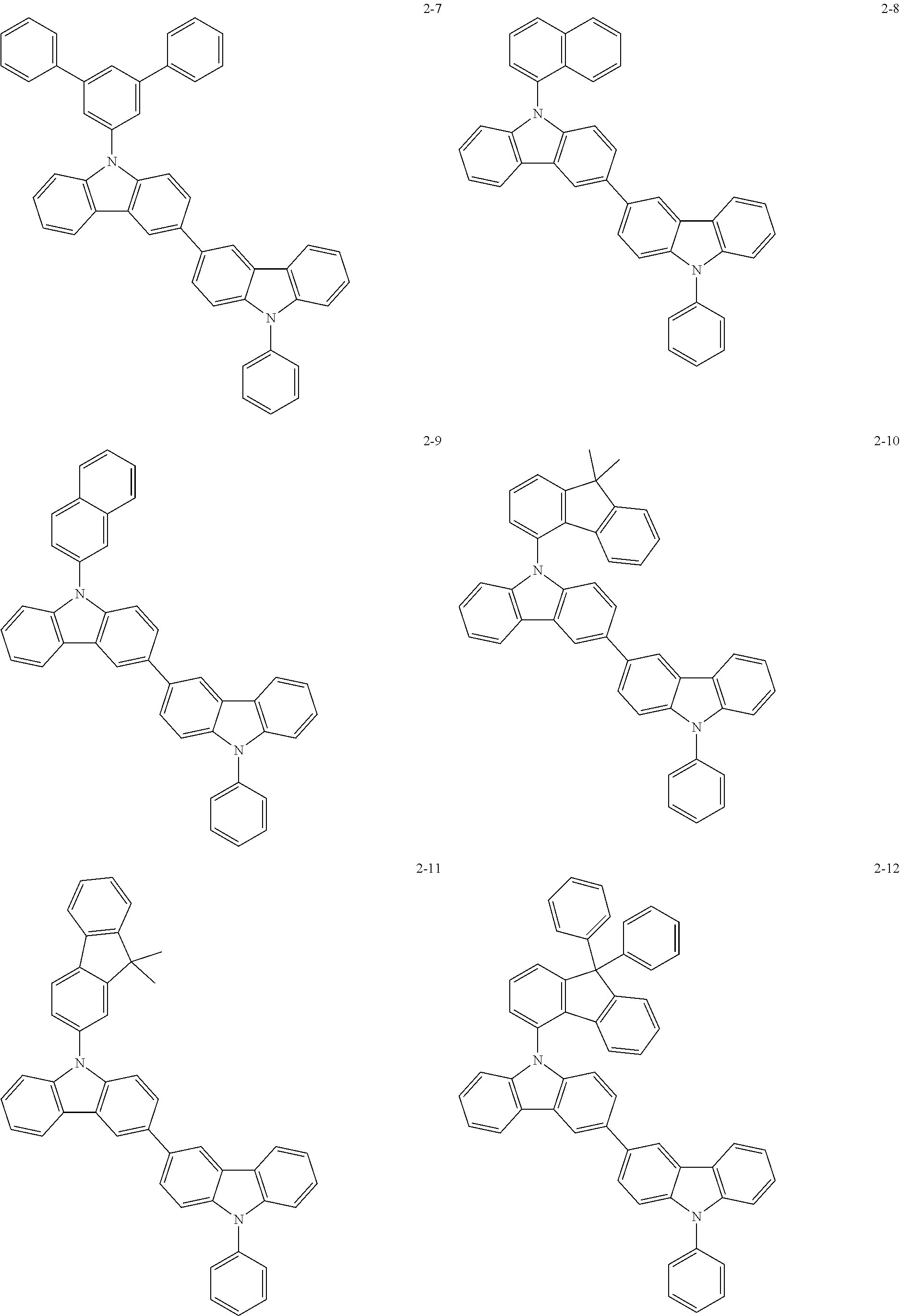 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

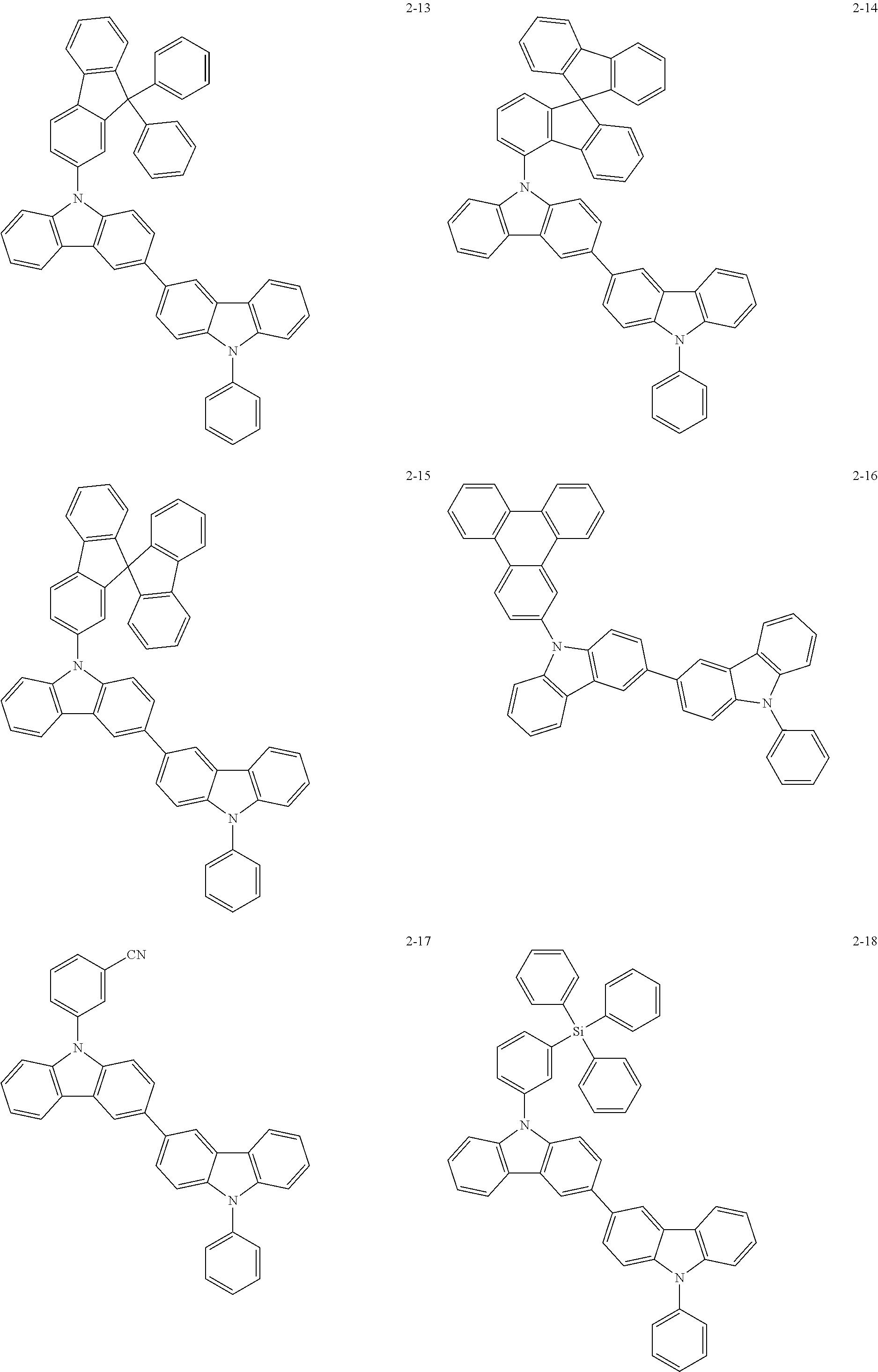

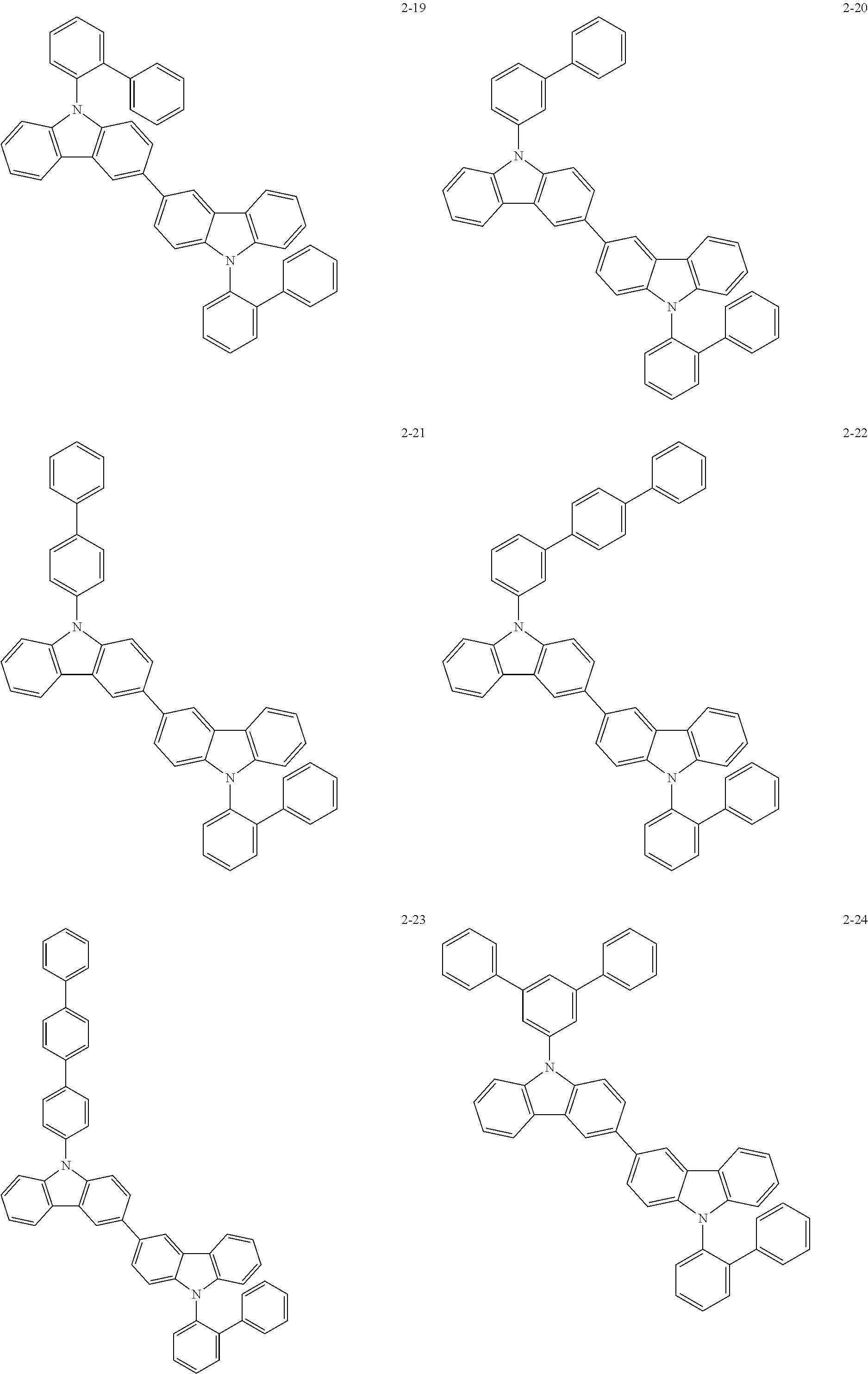

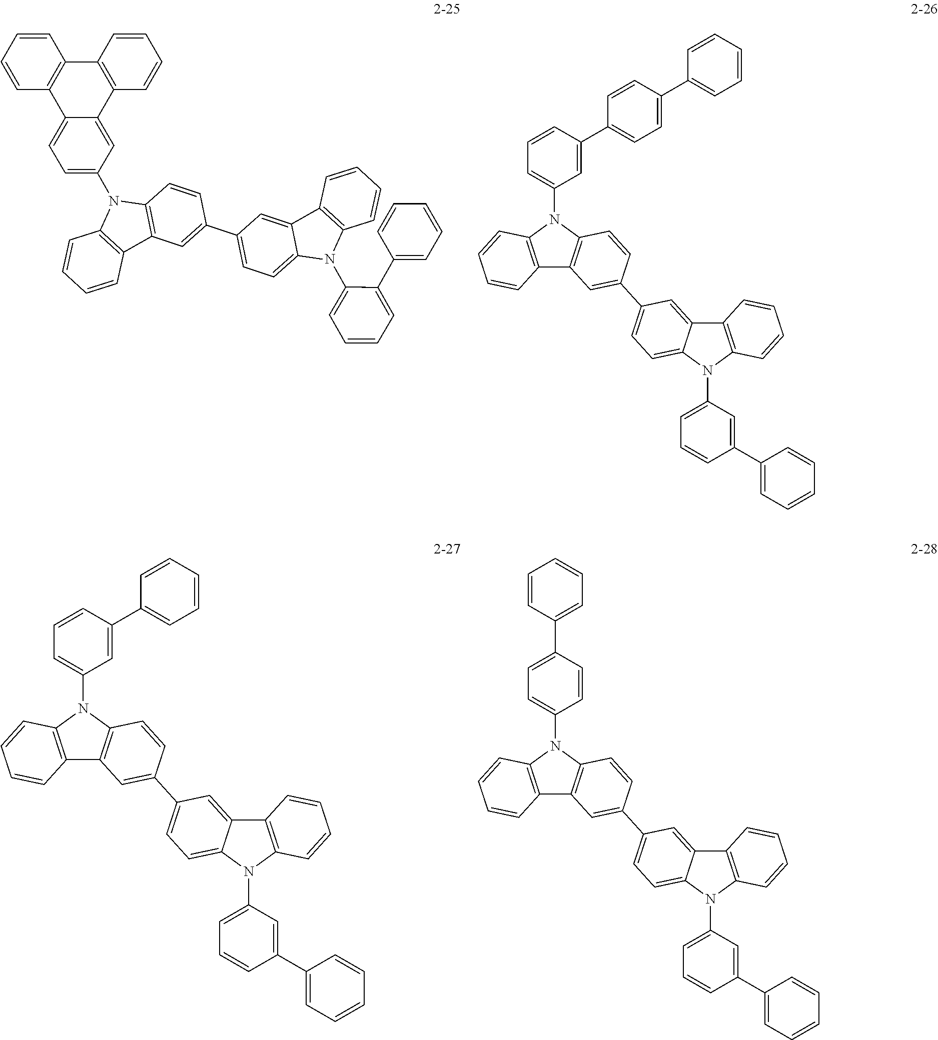

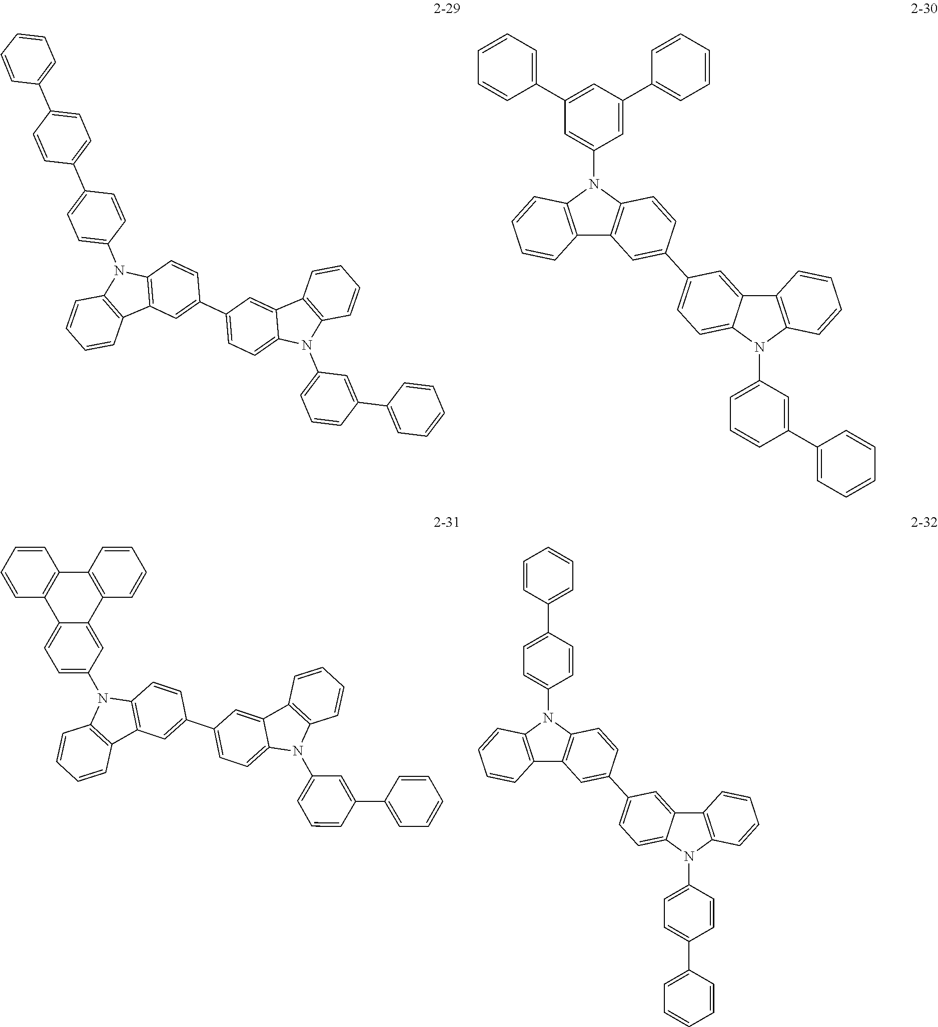 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

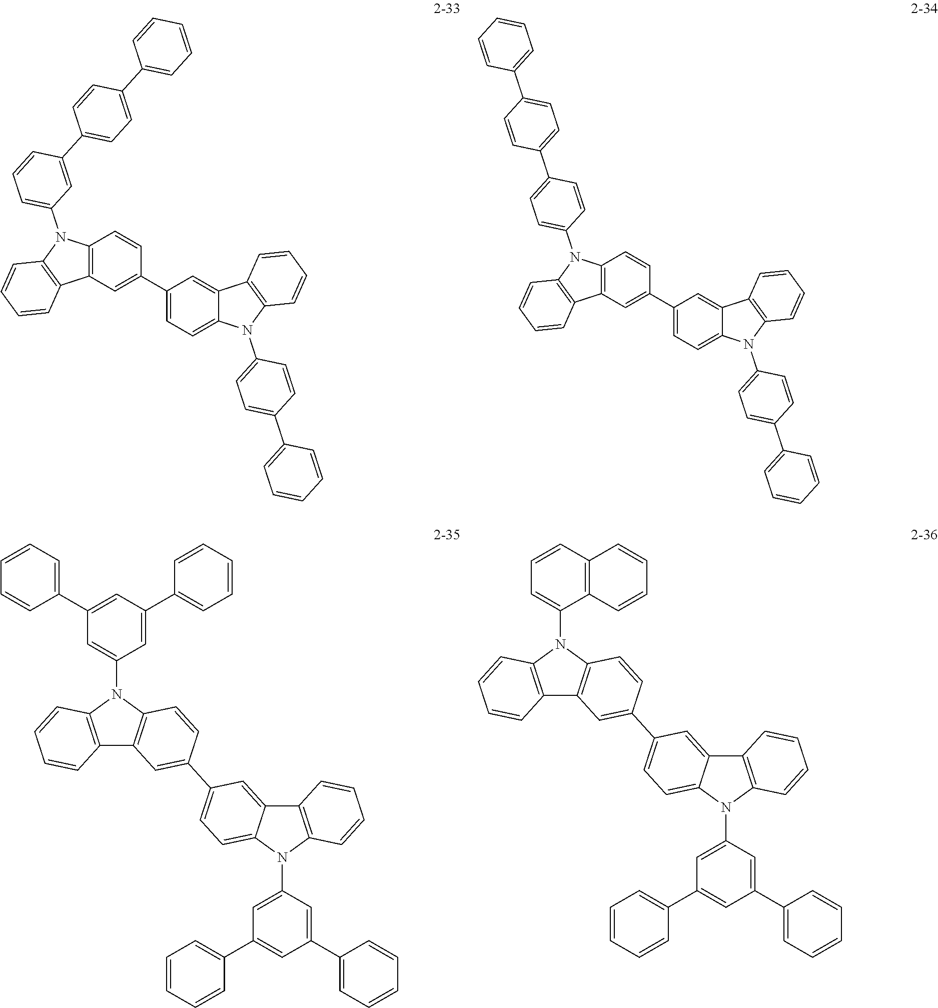 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

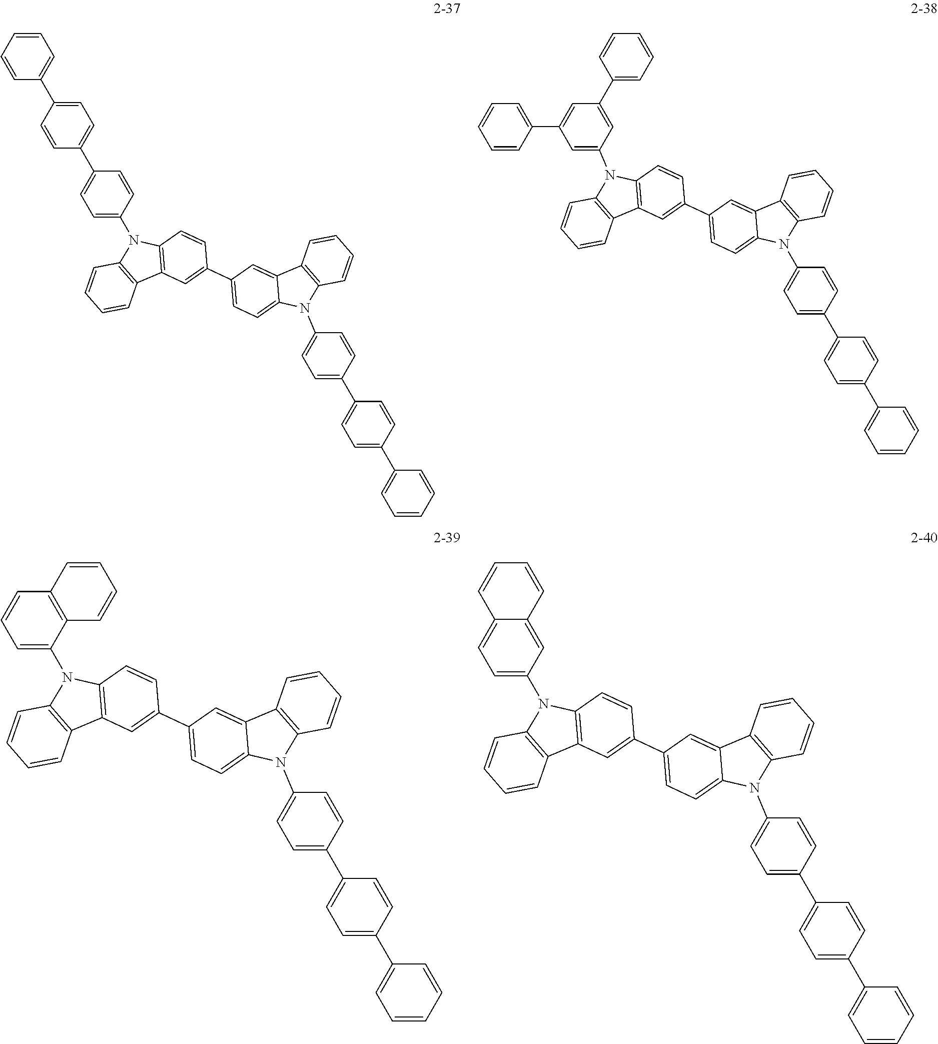 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

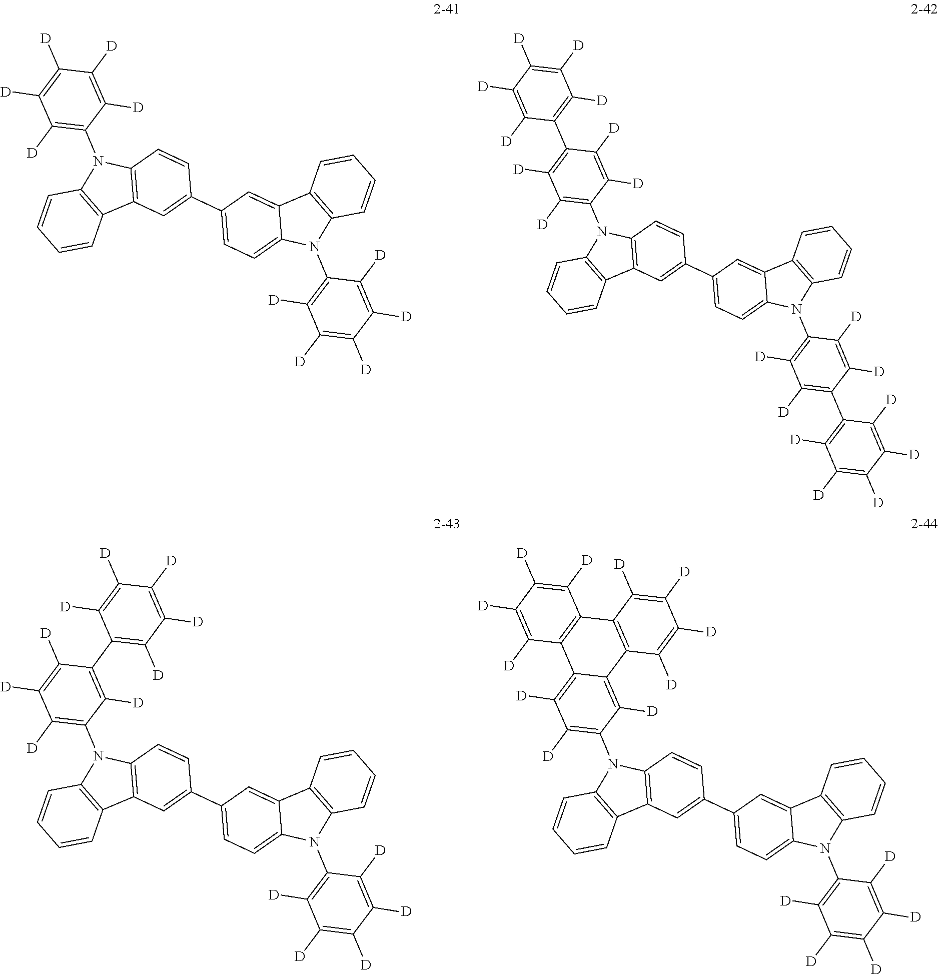

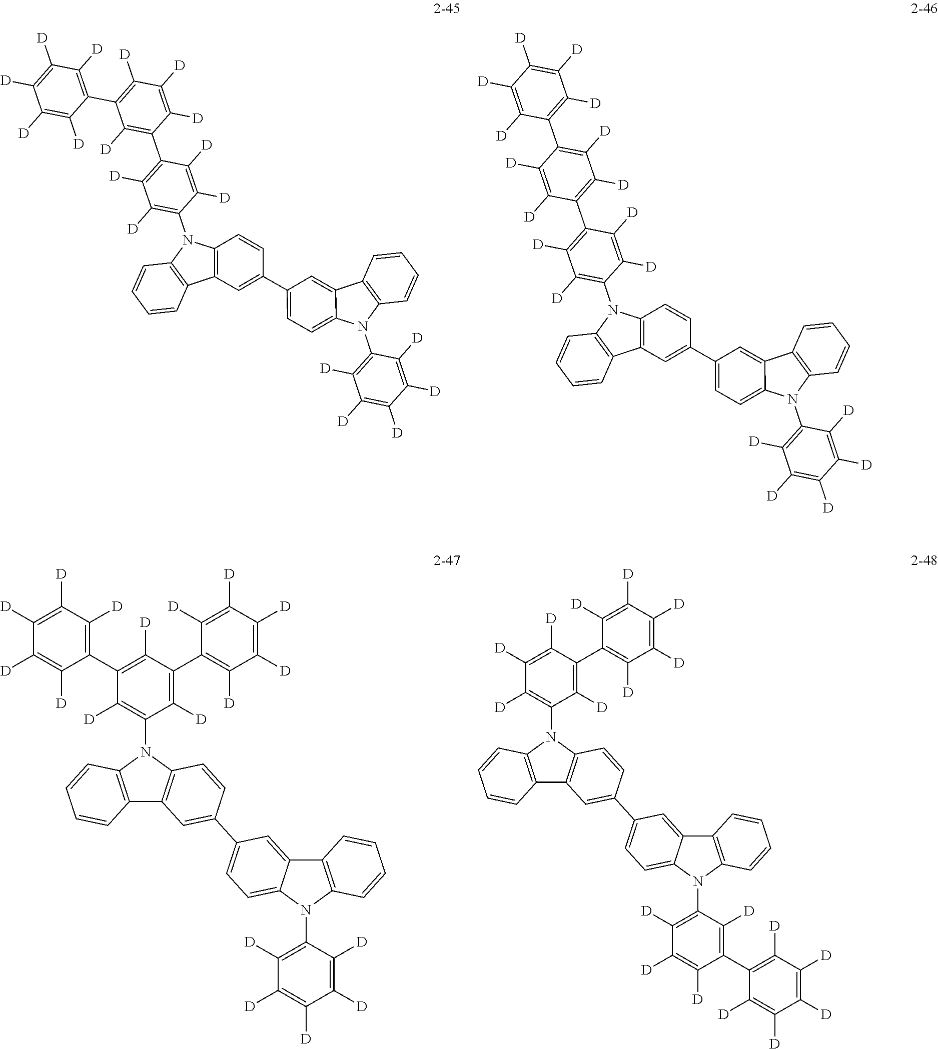

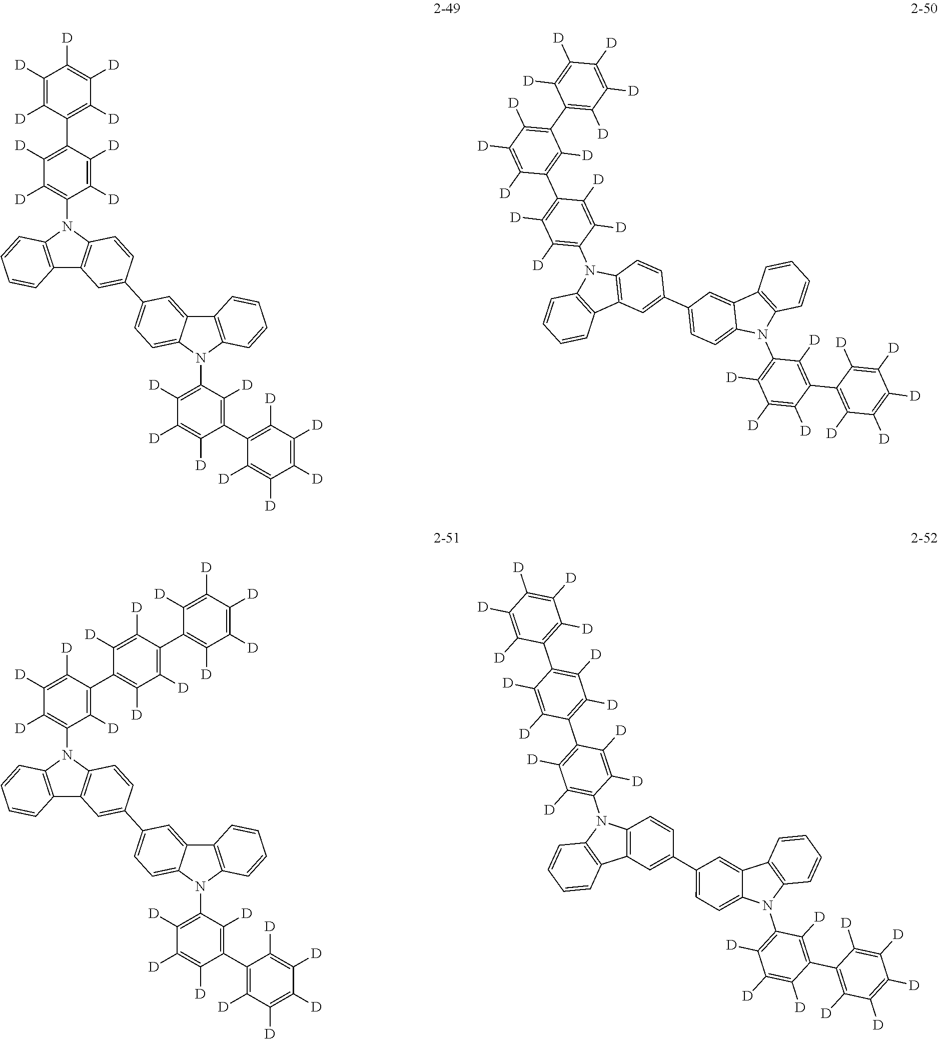

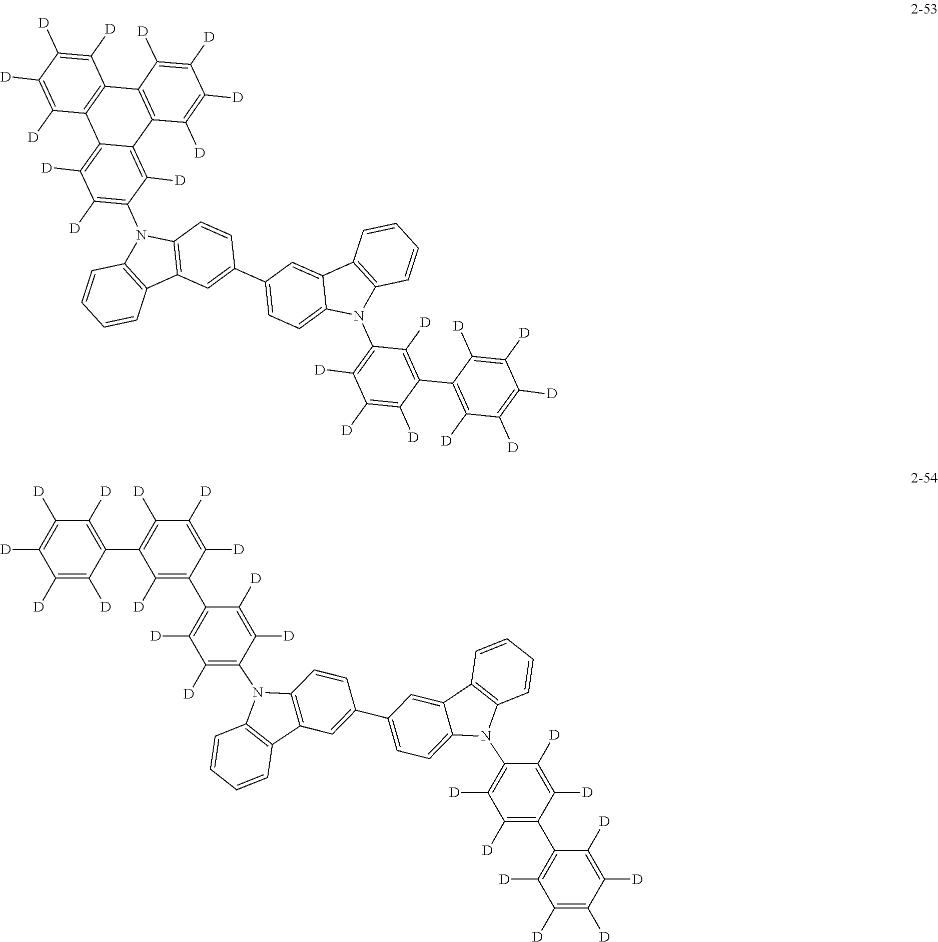 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c(-c7c([2H])c([2H])c([2H])c([2H])c7[2H])c6[2H])c([2H])c5[2H])ccc43)c([2H])c2[2H])c([2H])c1[2H]]

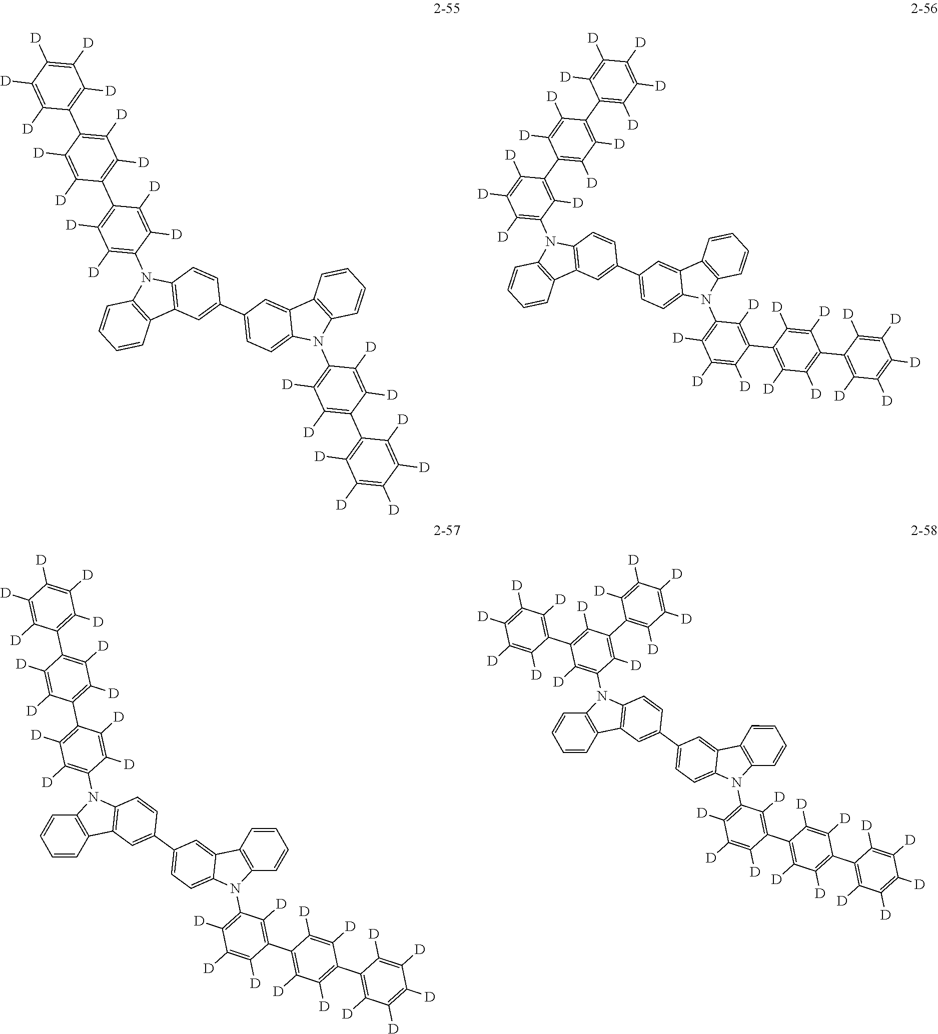

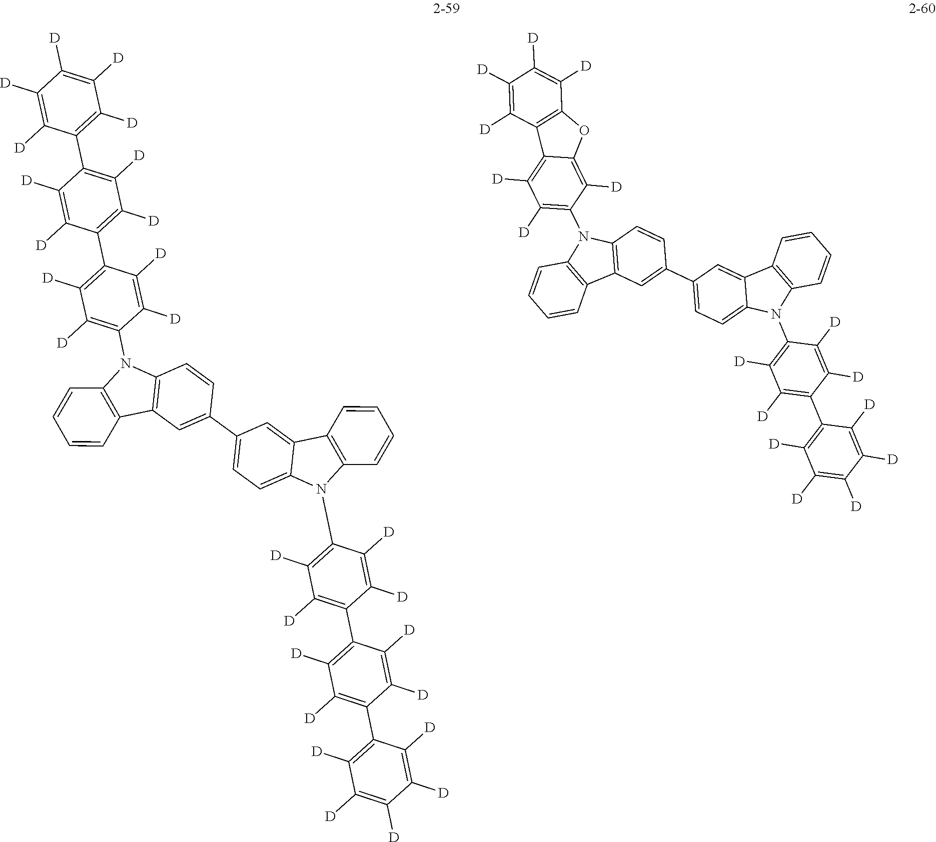 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c(-c8c([2H])c([2H])c([2H])c([2H])c8[2H])c([2H])c7[2H])c([2H])c6[2H])ccc54)c([2H])c3[2H])c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H]]

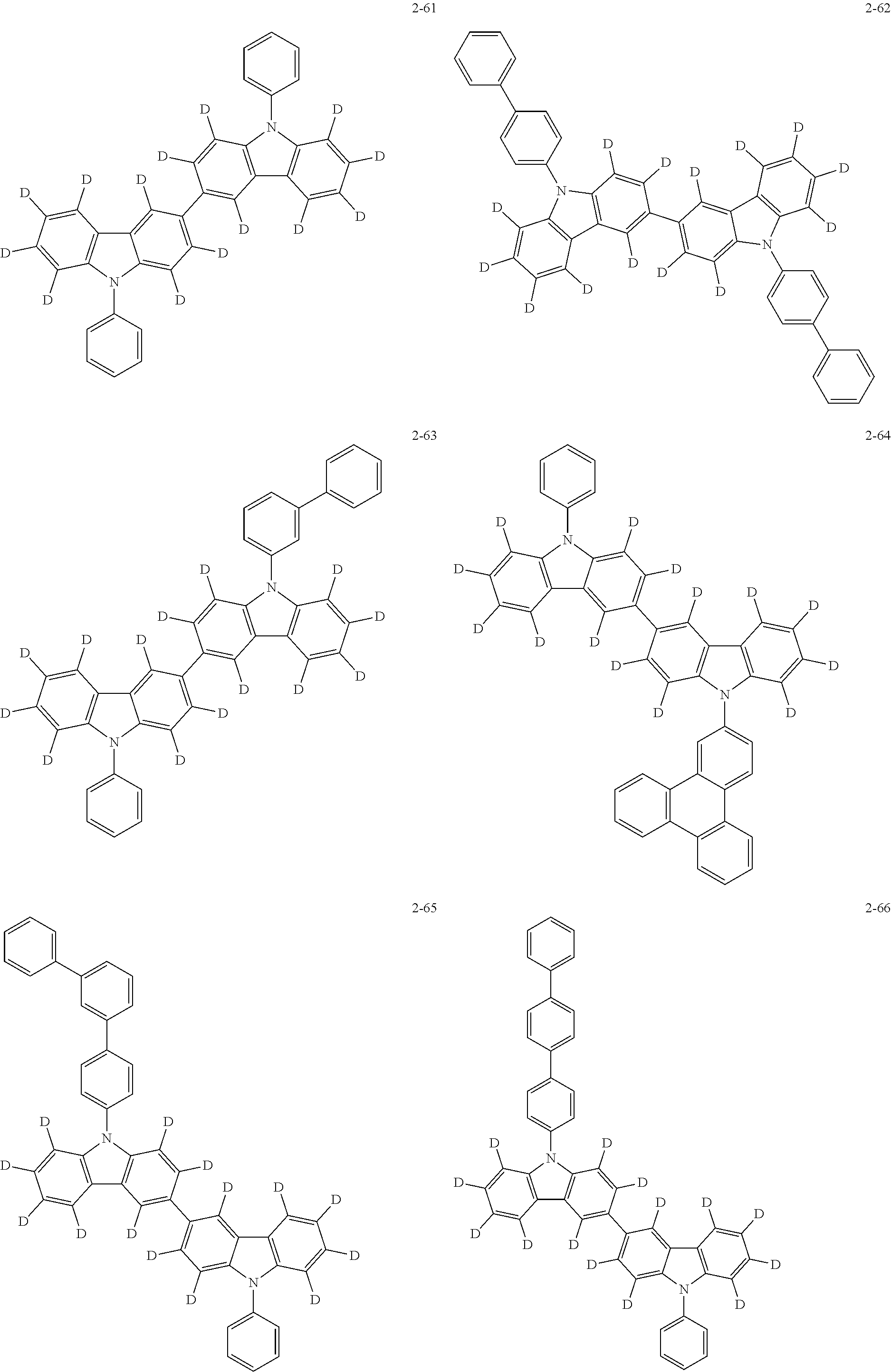

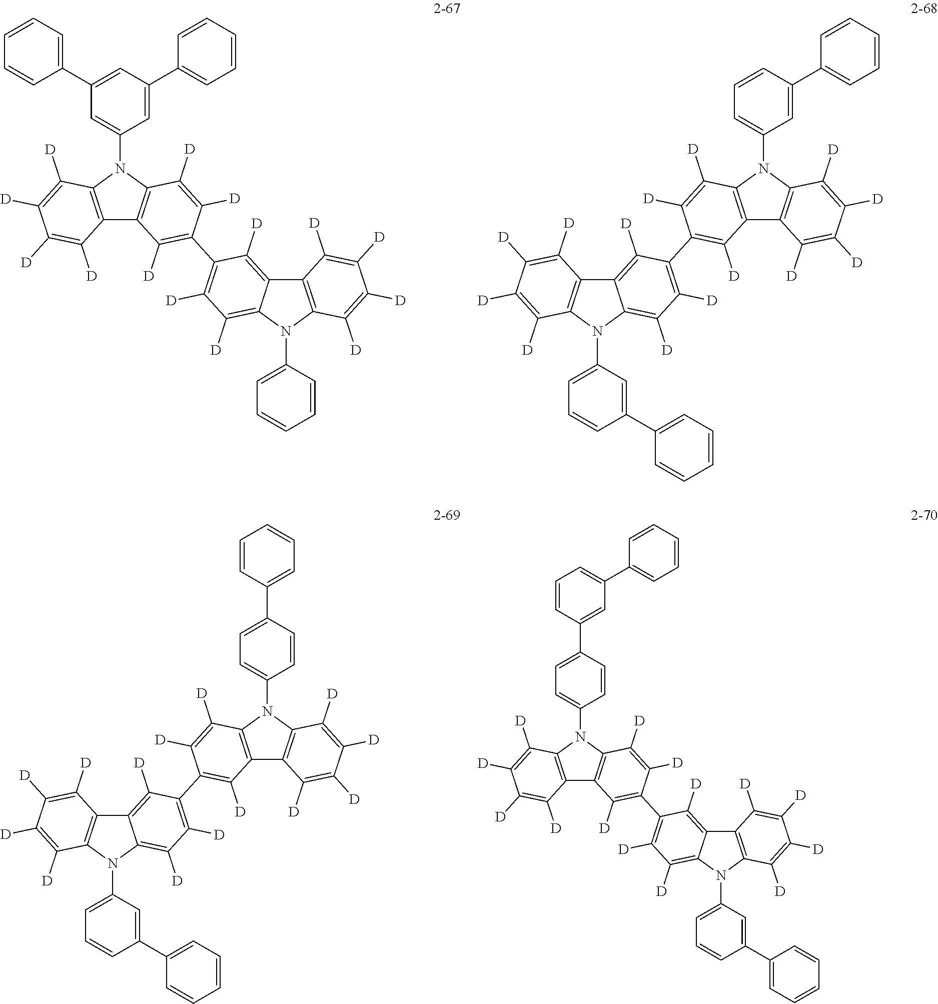

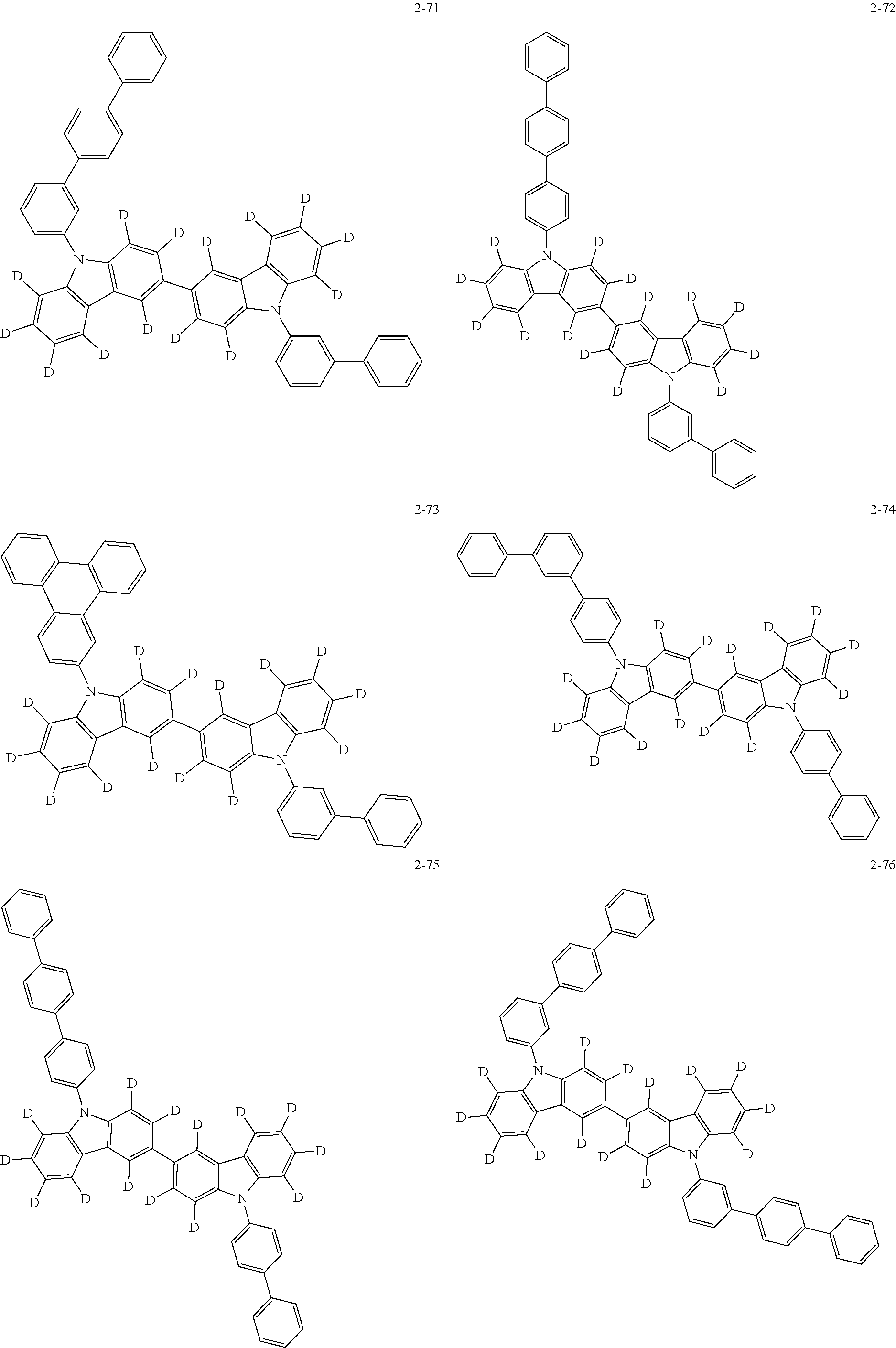

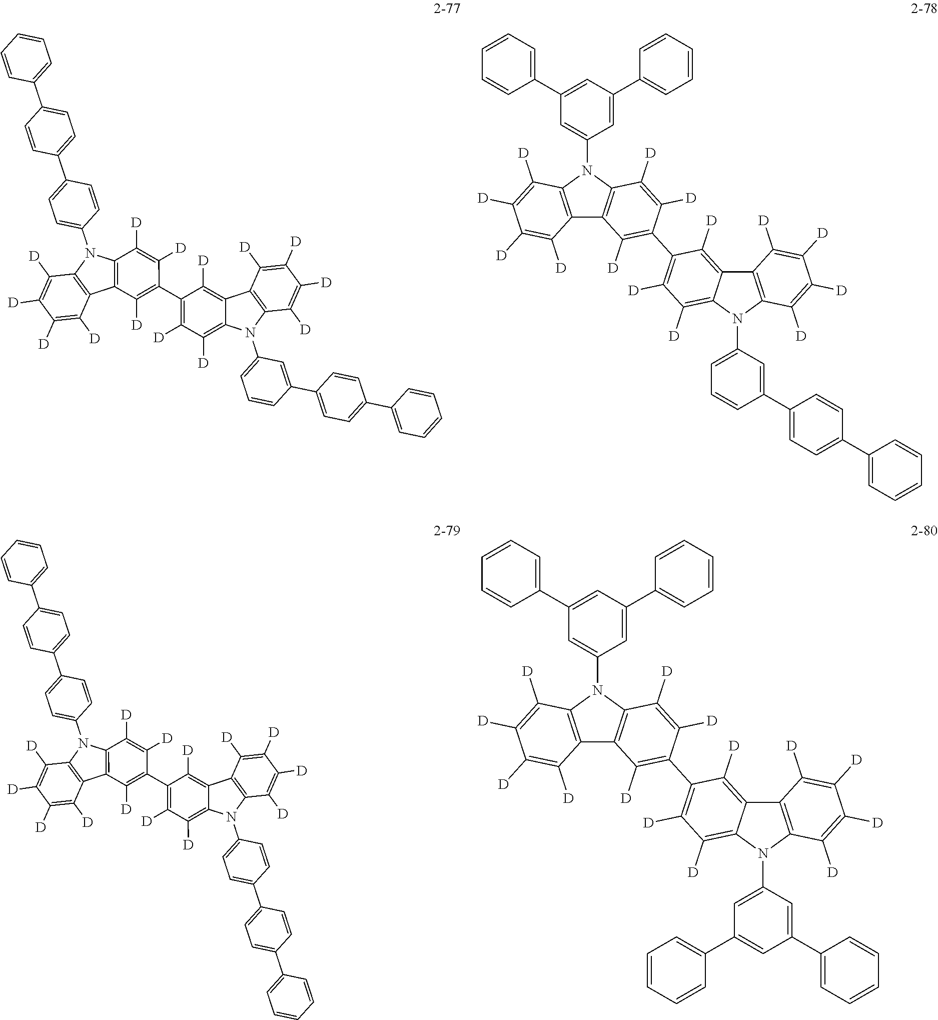

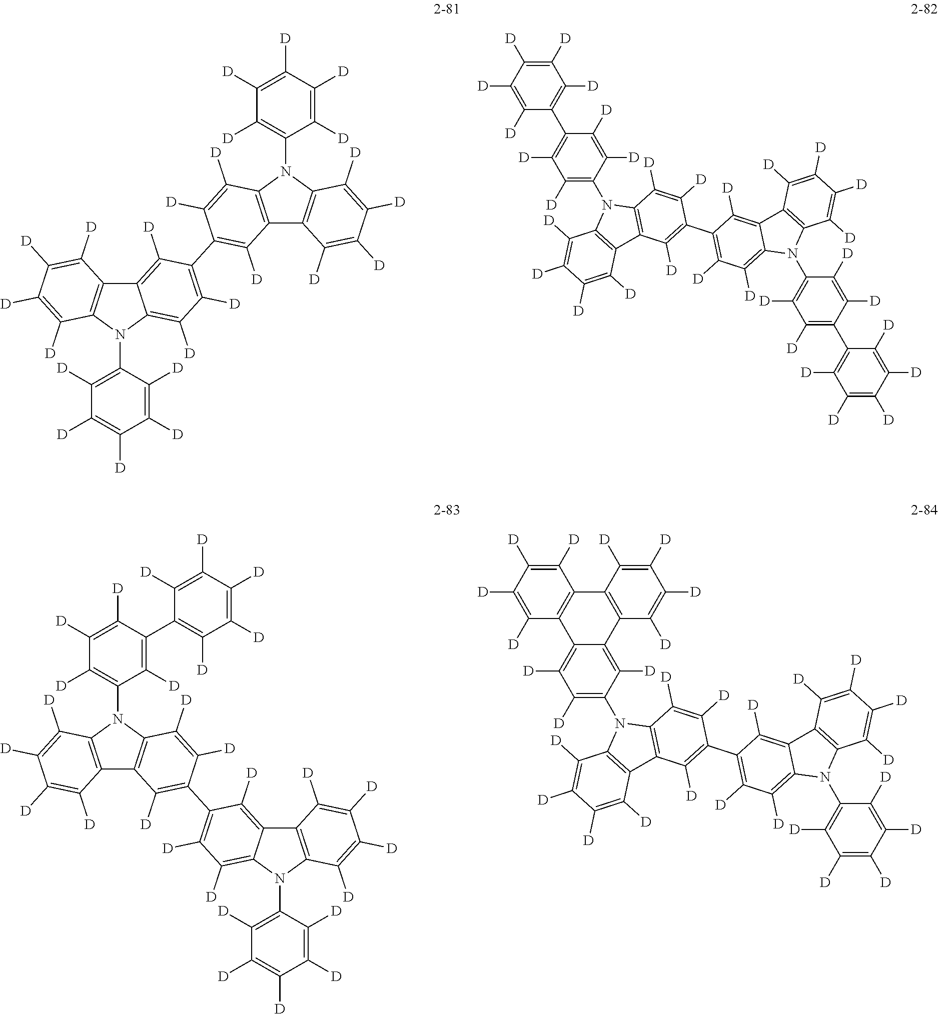

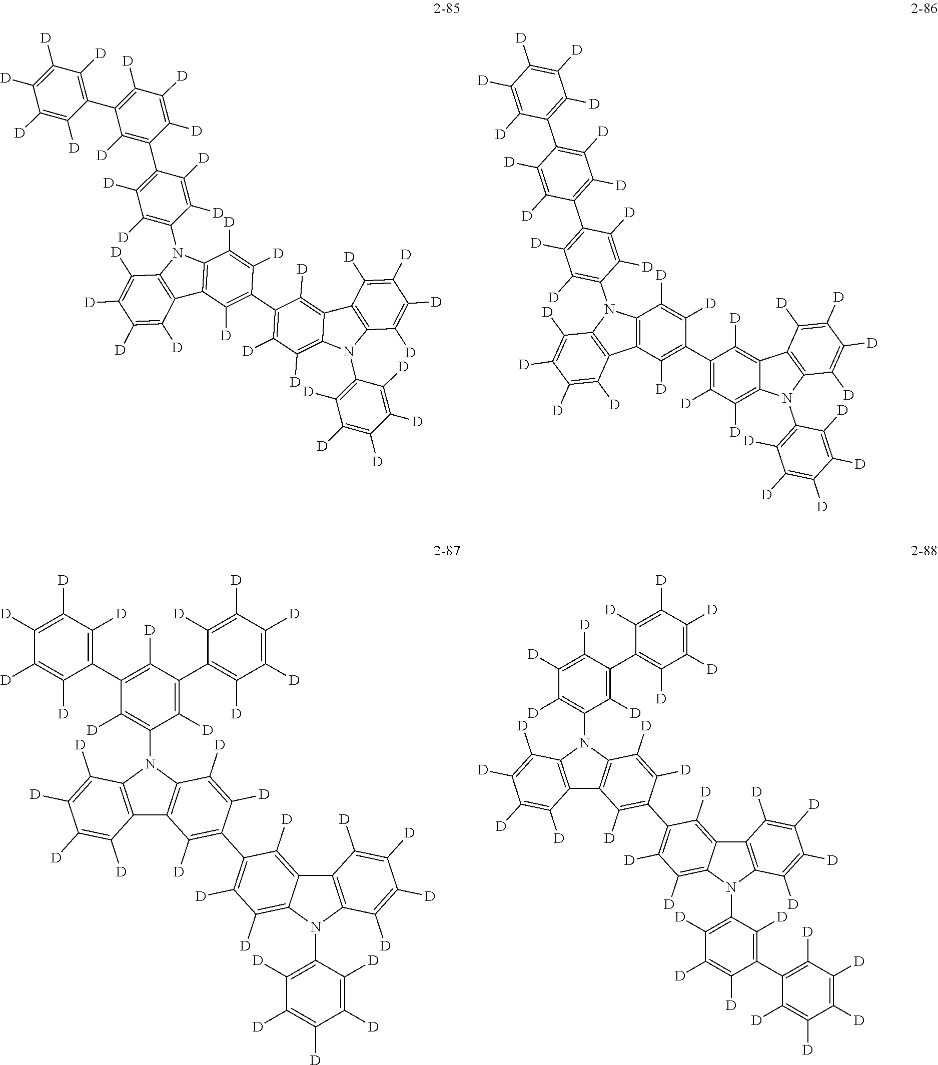

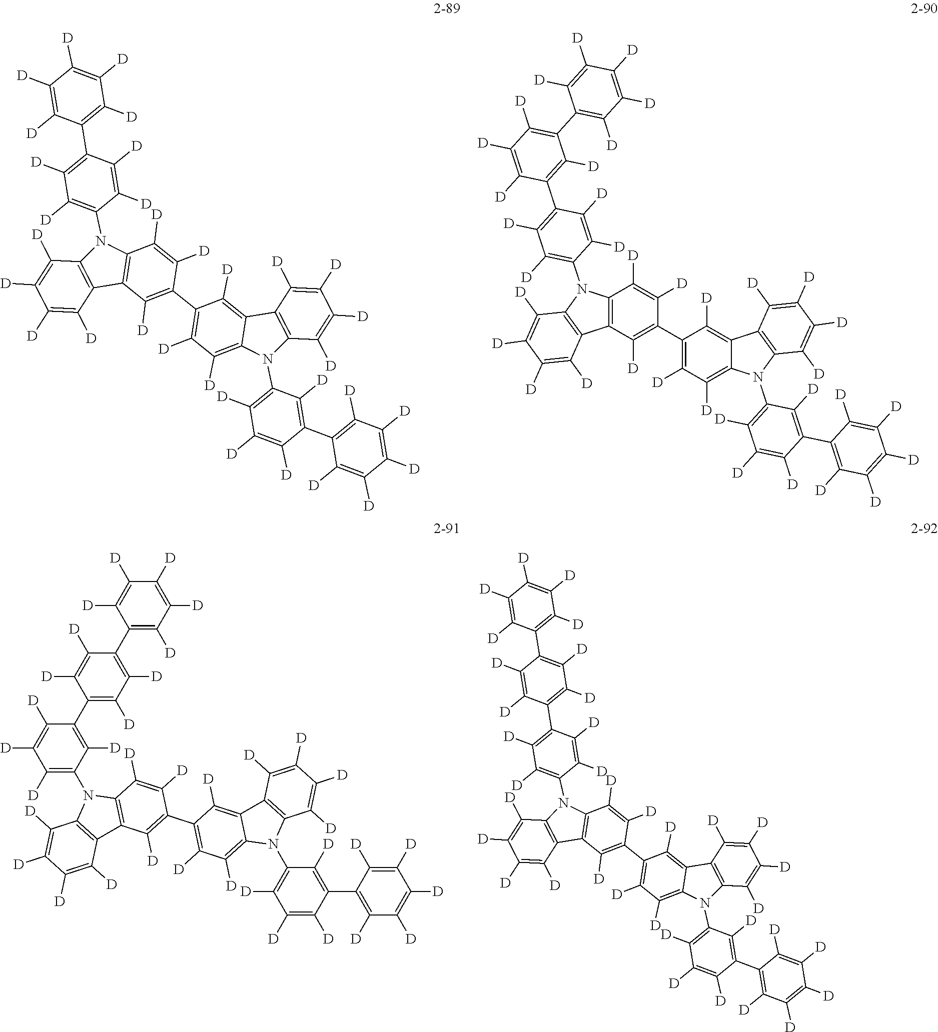

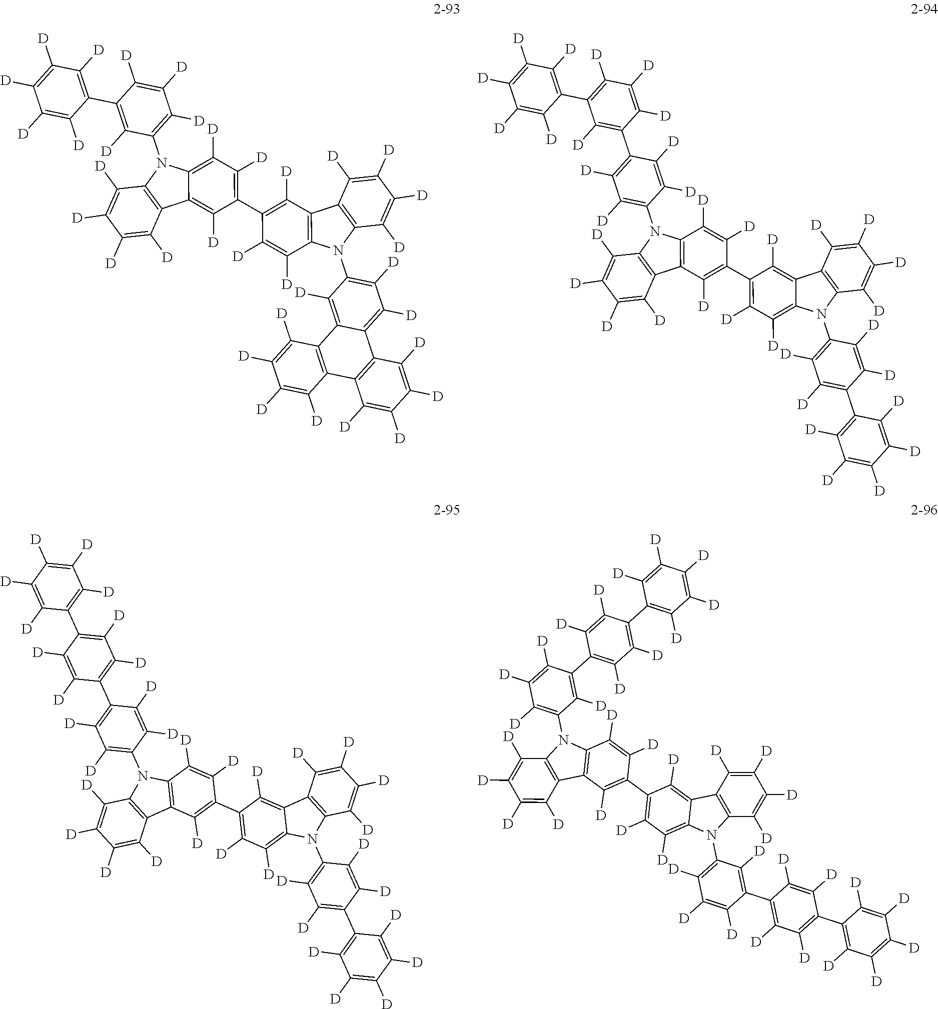

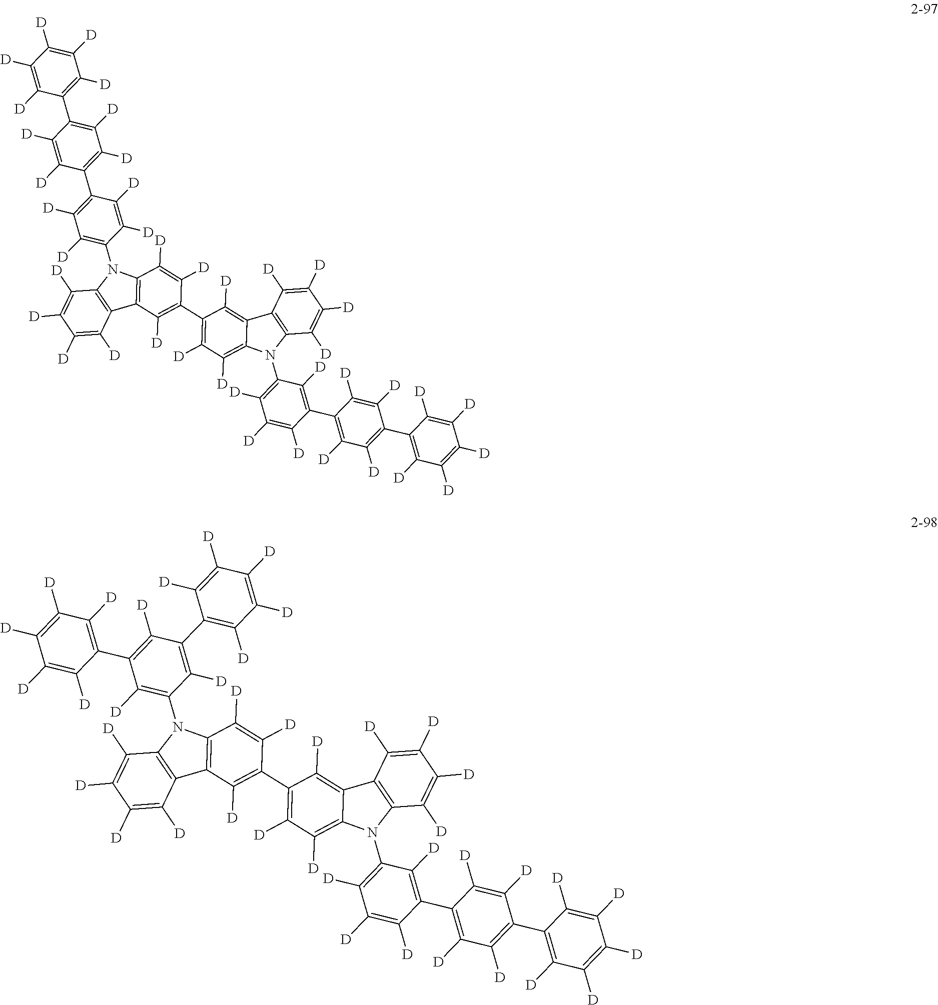

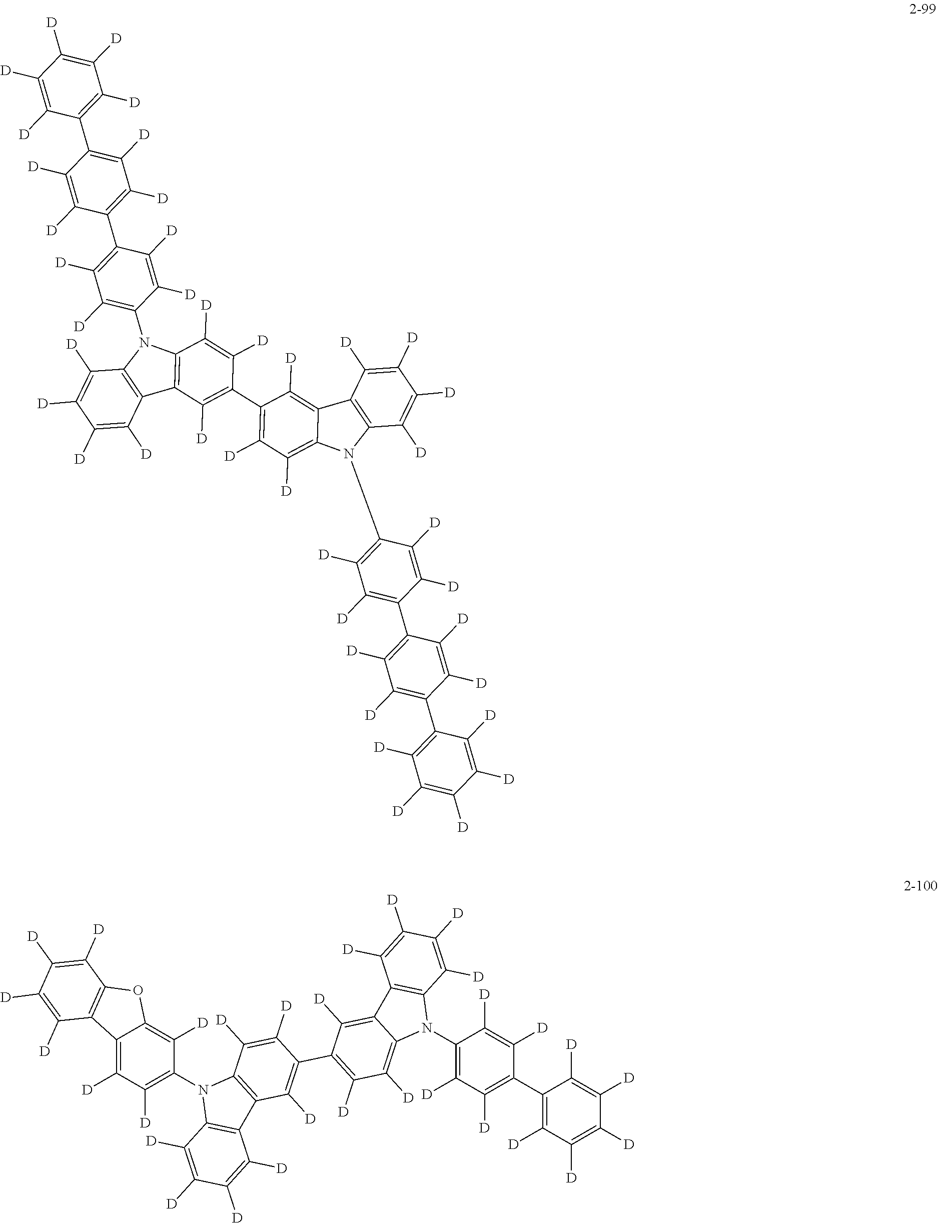

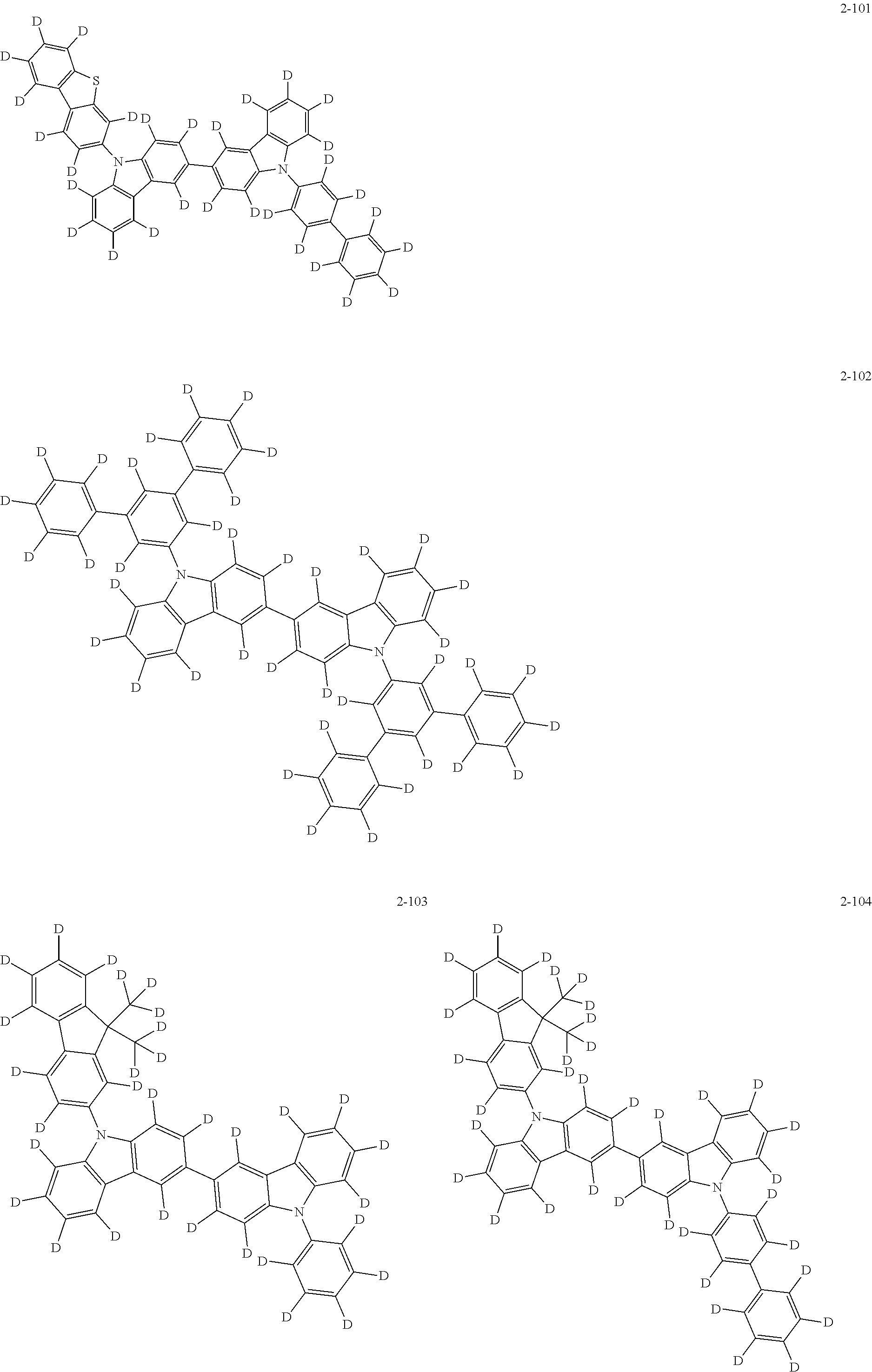

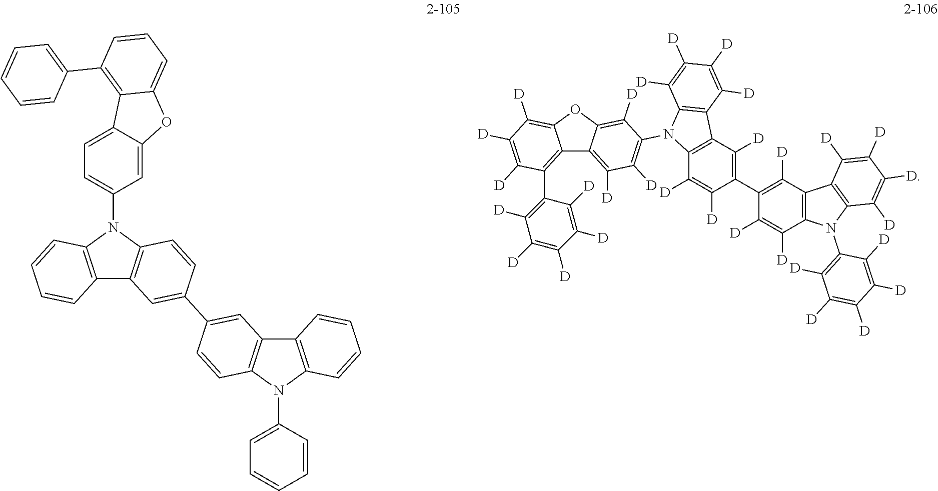

12. The organic light-emitting device according to claim 10, wherein Chemical Formula 4 is represented by any one of the following compounds:

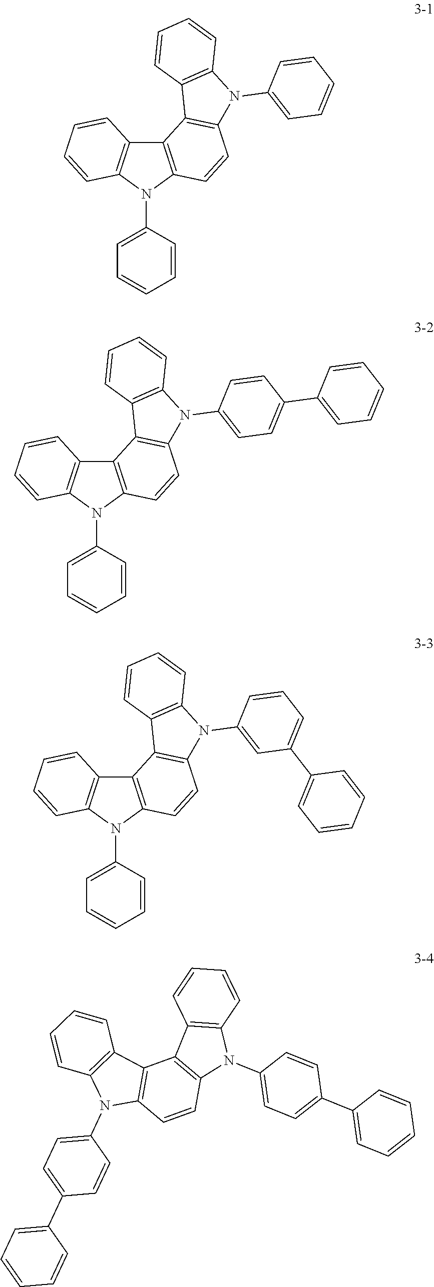

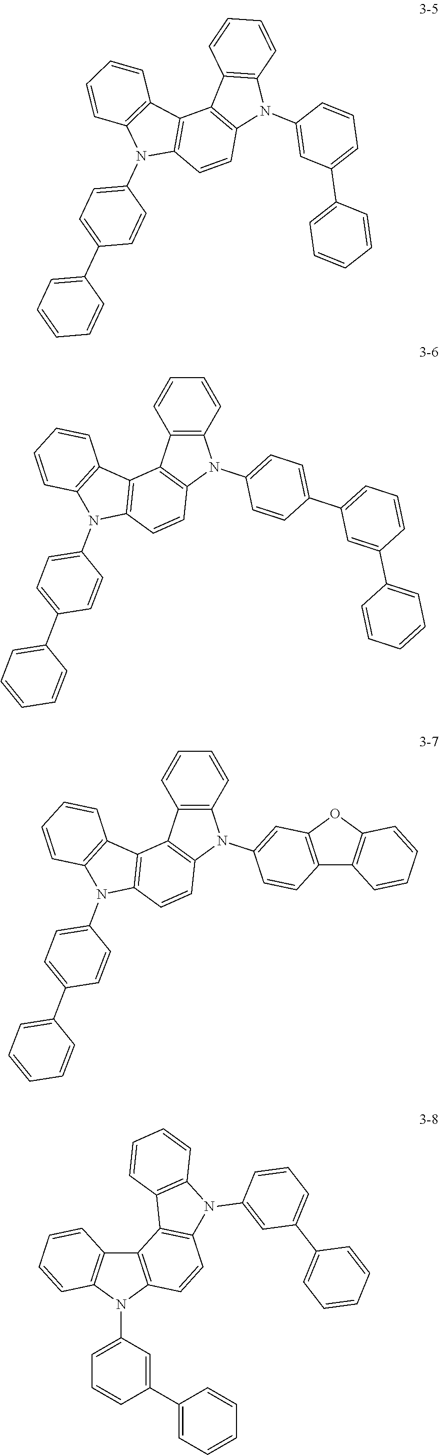 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2cccc(-n3c4ccccc4c4c5c6ccccc6n(-c6cccc(-c7ccccc7)c6)c5ccc43)c2)cc1]

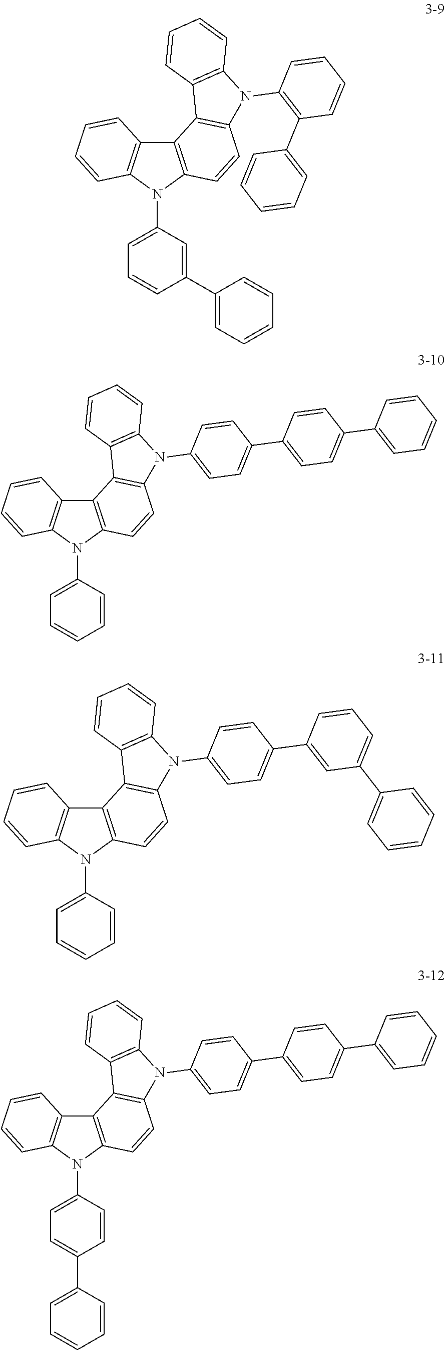 [SMILES: c1ccc(-c2cccc(-n3c4ccccc4c4c5c6ccccc6n(-c6ccccc6-c6ccccc6)c5ccc43)c2)cc1 ; c1ccc(-c2ccc(-c3ccc(-n4c5ccccc5c5c6c7ccccc7n(-c7ccccc7)c6ccc54)cc3)cc2)cc1 ; c1ccc(-c2cccc(-c3ccc(-n4c5ccccc5c5c6c7ccccc7n(-c7ccccc7)c6ccc54)cc3)c2)cc1 ; c1ccc(-c2ccc(-c3ccc(-n4c5ccccc5c5c6c7ccccc7n(-c7ccc(-c8ccccc8)cc7)c6ccc54)cc3)cc2)cc1]

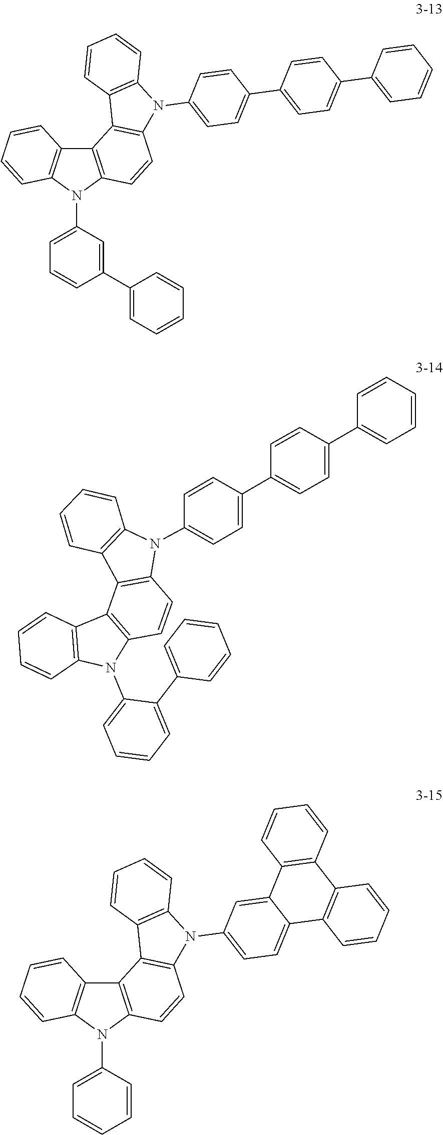 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2ccc(-c3ccc(-n4c5ccccc5c5c6c7ccccc7n(-c7ccccc7-c7ccccc7)c6ccc54)cc3)cc2)cc1 ; c1ccc(-n2c3ccccc3c3c4c5ccccc5n(-c5ccc6c7ccccc7c7ccccc7c6c5)c4ccc32)cc1]

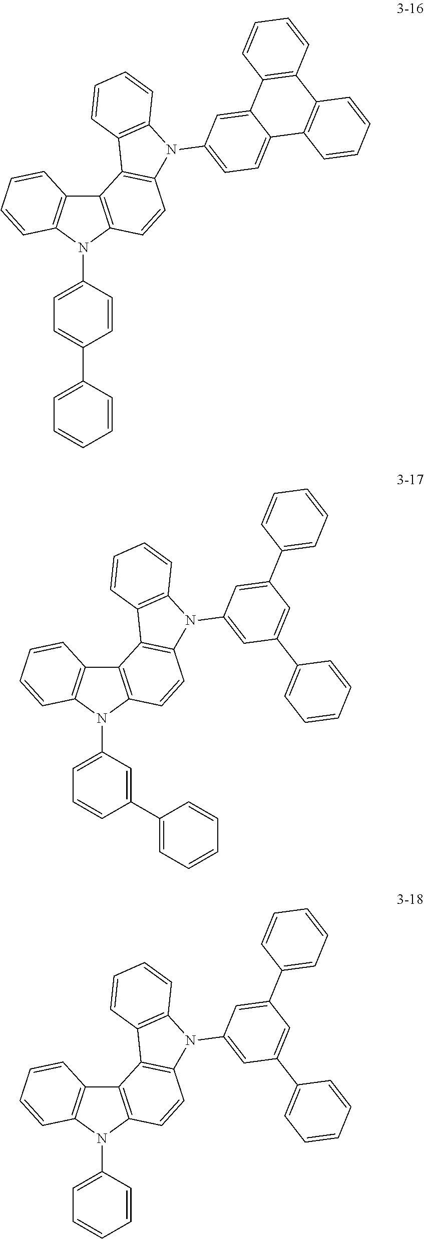 [SMILES: c1ccc(-c2ccc(-n3c4ccccc4c4c5c6ccccc6n(-c6ccc7c8ccccc8c8ccccc8c7c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6ccccc6)c5ccc43)c2)cc1]

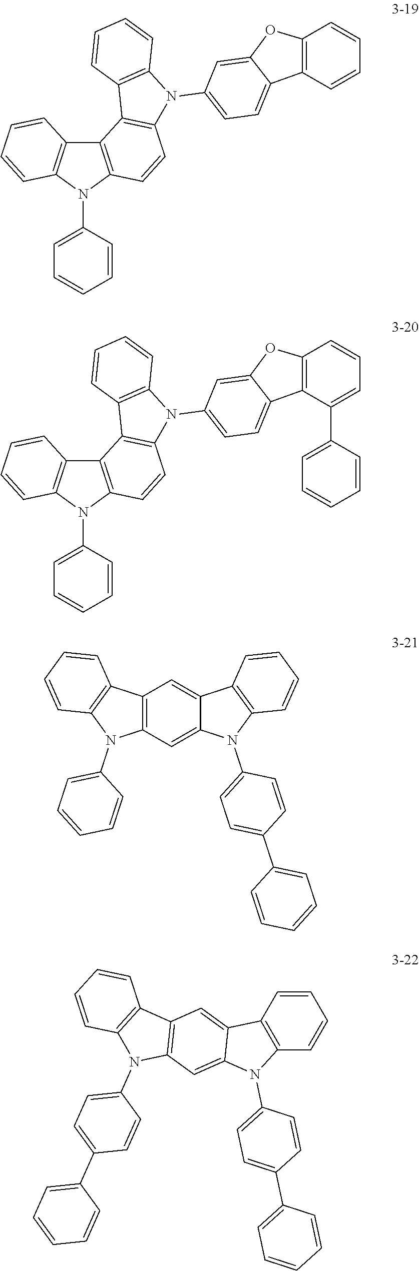

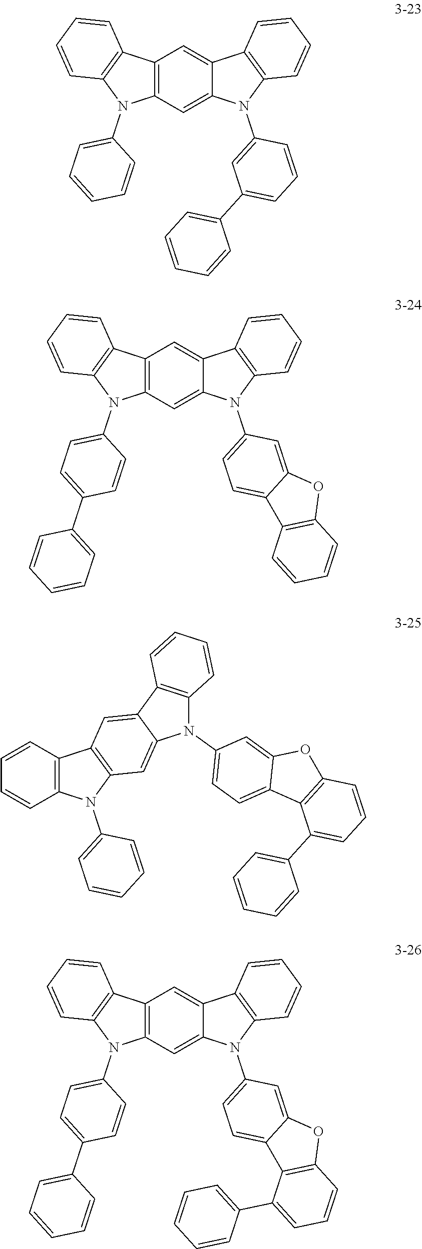

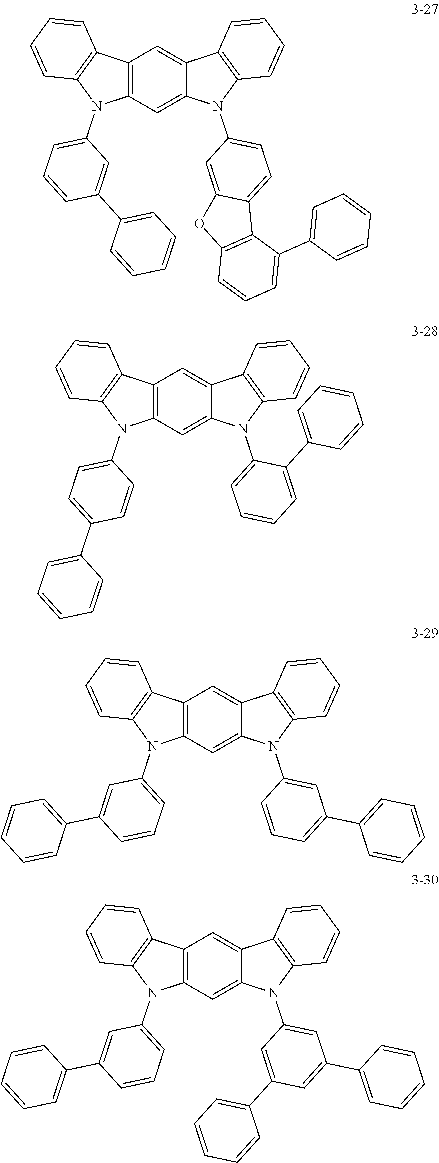

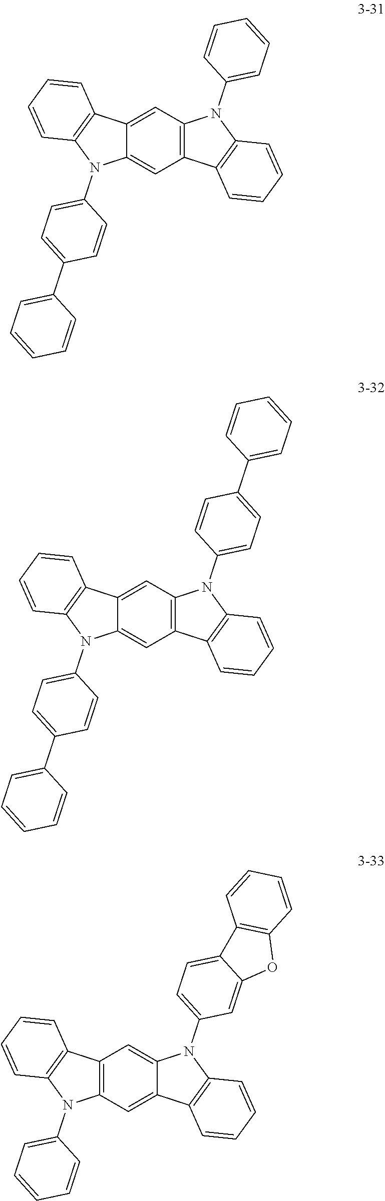 [SMILES: c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1 ; c1ccc(-n2c3ccccc3c3cc4c(cc32)c2ccccc2n4-c2ccc3c(c2)oc2ccccc23)cc1]

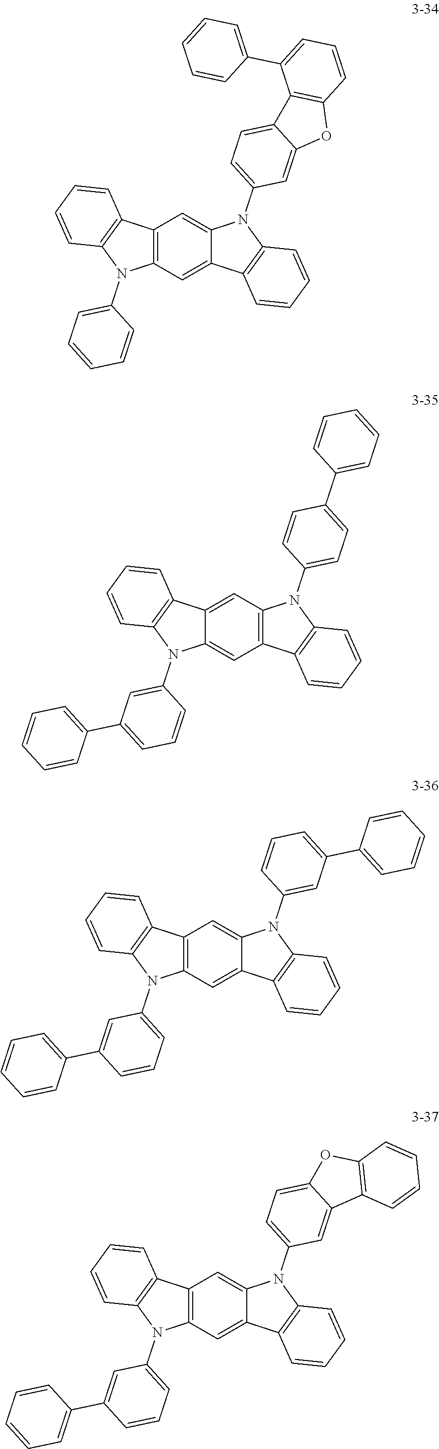 [SMILES: c1ccc(-c2cccc3oc4cc(-n5c6ccccc6c6cc7c(cc65)c5ccccc5n7-c5ccccc5)ccc4c23)cc1 ; c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1 ; c1ccc(-c2cccc(-n3c4ccccc4c4cc5c(cc43)c3ccccc3n5-c3ccc4oc5ccccc5c4c3)c2)cc1]

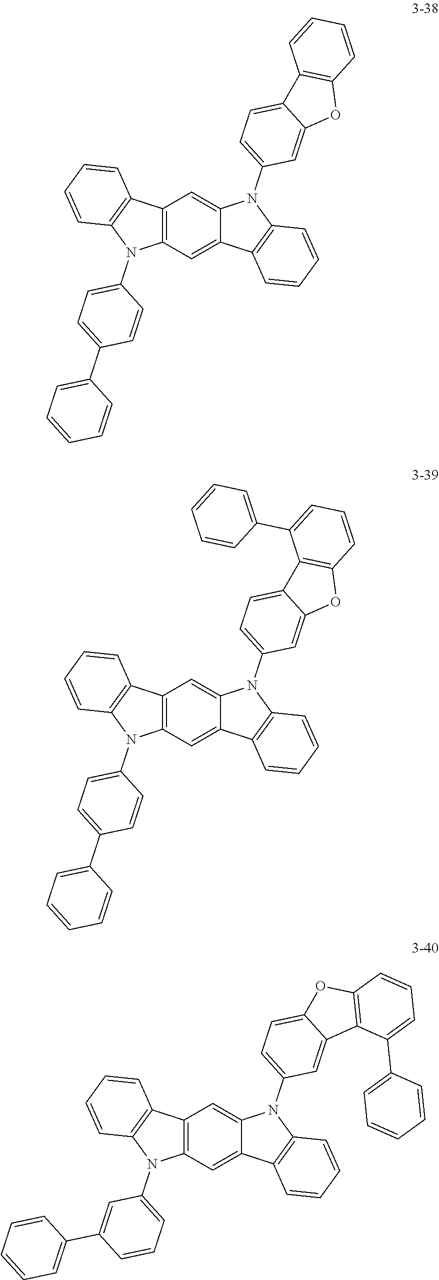 [SMILES: c1ccc(-c2ccc(-n3c4ccccc4c4cc5c(cc43)c3ccccc3n5-c3ccc4c(c3)oc3ccccc34)cc2)cc1 ; c1ccc(-c2ccc(-n3c4ccccc4c4cc5c(cc43)c3ccccc3n5-c3ccc4c(c3)oc3cccc(-c5ccccc5)c34)cc2)cc1 ; c1ccc(-c2cccc(-n3c4ccccc4c4cc5c(cc43)c3ccccc3n5-c3ccc4oc5cccc(-c6ccccc6)c5c4c3)c2)cc1]

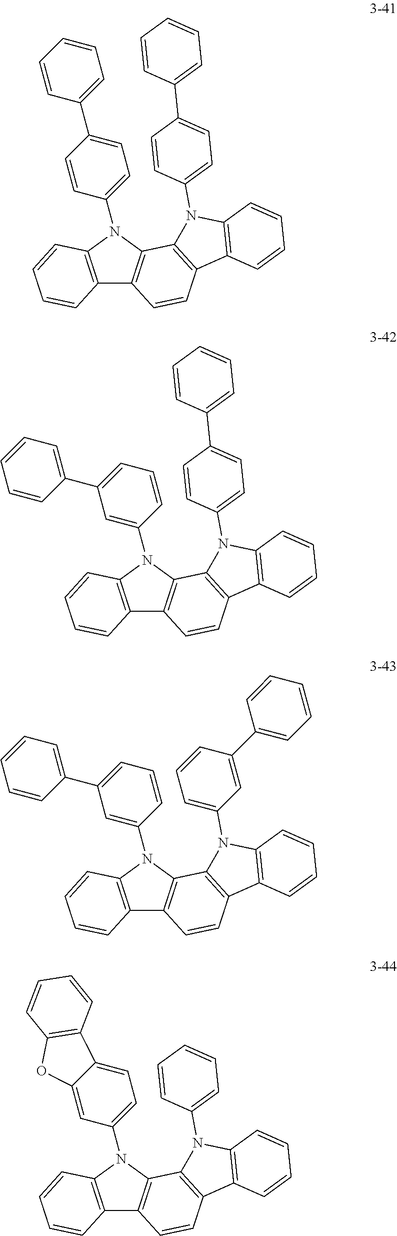 [SMILES: c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-n2c3ccccc3c3ccc4c5ccccc5n(-c5ccc6c(c5)oc5ccccc56)c4c32)cc1]

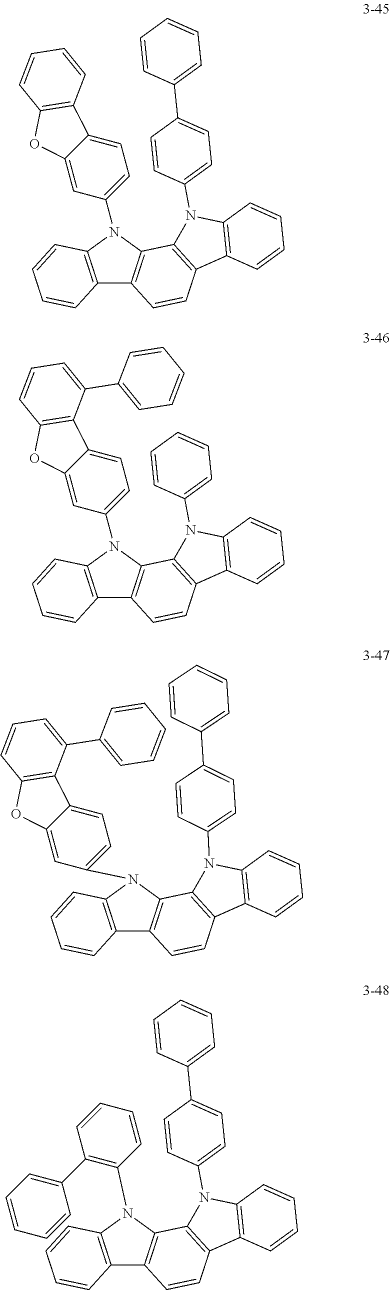

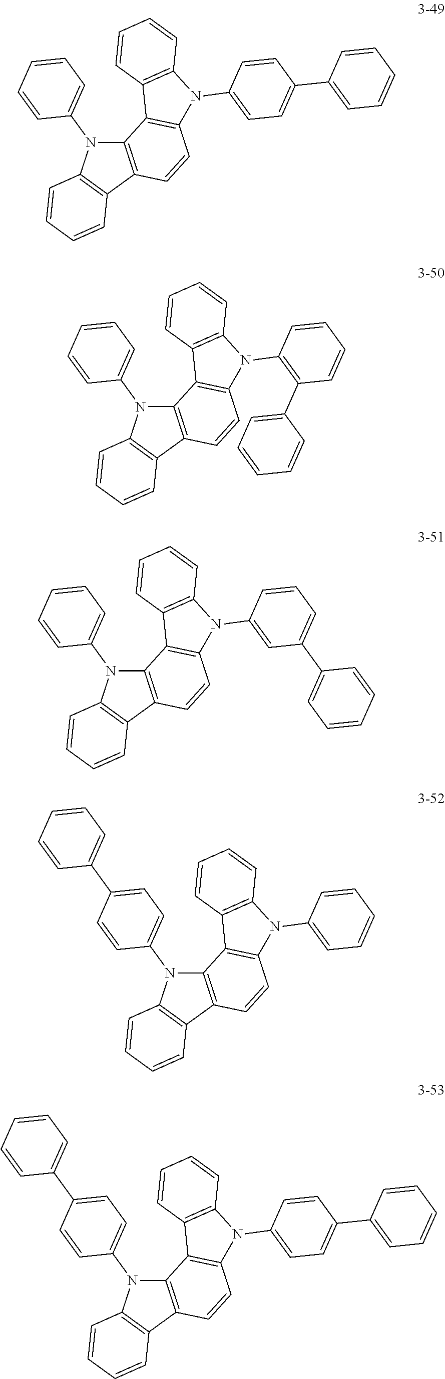

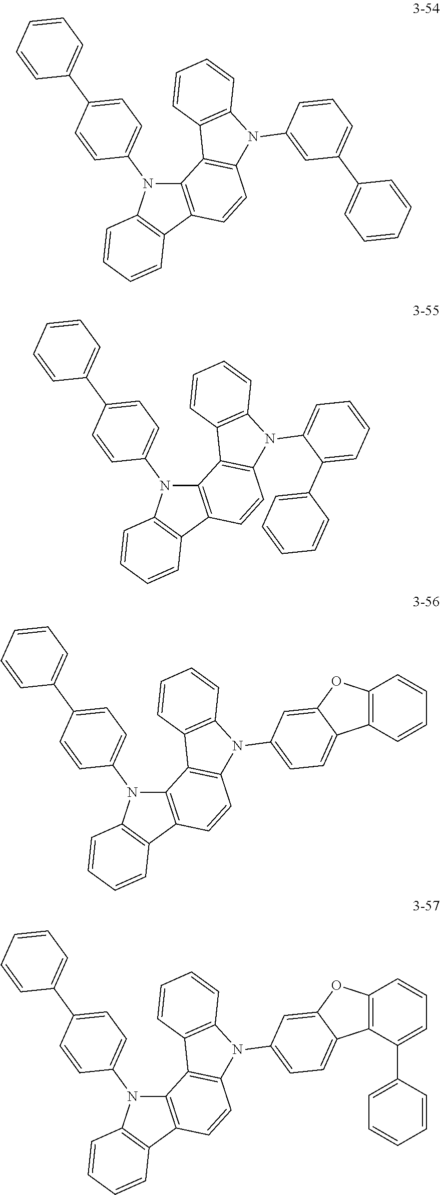 [SMILES: c1ccc(-c2ccc(-n3c4ccccc4c4ccc5c(c6ccccc6n5-c5cccc(-c6ccccc6)c5)c43)cc2)cc1 ; c1ccc(-c2ccc(-n3c4ccccc4c4ccc5c(c6ccccc6n5-c5ccccc5-c5ccccc5)c43)cc2)cc1 ; c1ccc(-c2ccc(-n3c4ccccc4c4ccc5c(c6ccccc6n5-c5ccc6c(c5)oc5ccccc56)c43)cc2)cc1 ; c1ccc(-c2ccc(-n3c4ccccc4c4ccc5c(c6ccccc6n5-c5ccc6c(c5)oc5cccc(-c7ccccc7)c56)c43)cc2)cc1]

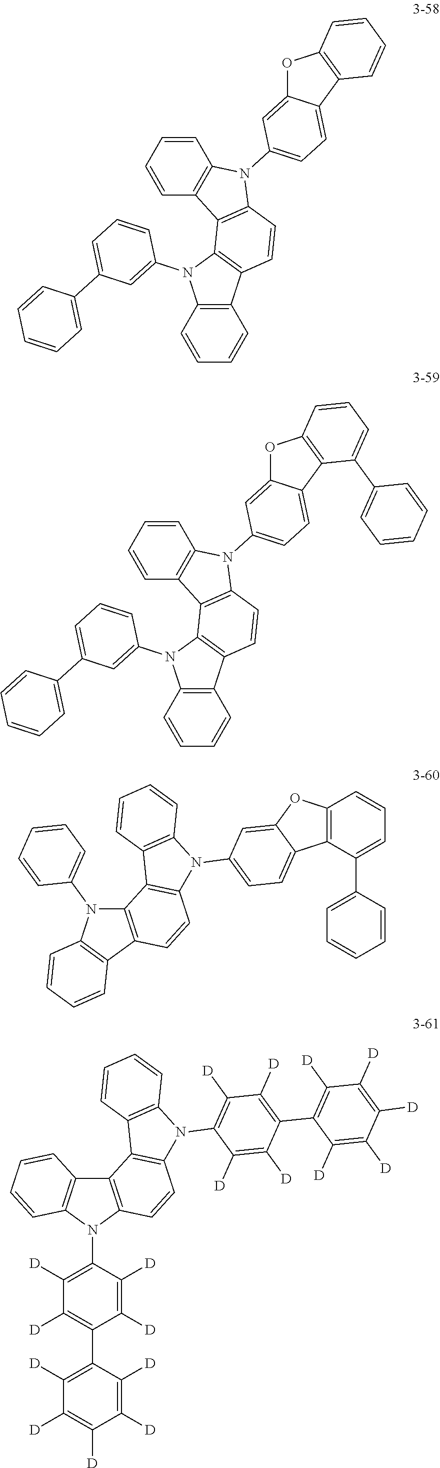 [SMILES: c1ccc(-c2cccc(-n3c4ccccc4c4ccc5c(c6ccccc6n5-c5ccc6c(c5)oc5ccccc56)c43)c2)cc1 ; c1ccc(-c2cccc(-n3c4ccccc4c4ccc5c(c6ccccc6n5-c5ccc6c(c5)oc5cccc(-c7ccccc7)c56)c43)c2)cc1 ; c1ccc(-c2cccc3oc4cc(-n5c6ccccc6c6c5ccc5c7ccccc7n(-c7ccccc7)c56)ccc4c23)cc1 ; [2H]c1c([2H])c([2H])c(-c2c([2H])c([2H])c(-n3c4ccccc4c4c5c6ccccc6n(-c6c([2H])c([2H])c(-c7c([2H])c([2H])c([2H])c([2H])c7[2H])c([2H])c6[2H])c5ccc43)c([2H])c2[2H])c([2H])c1[2H]]

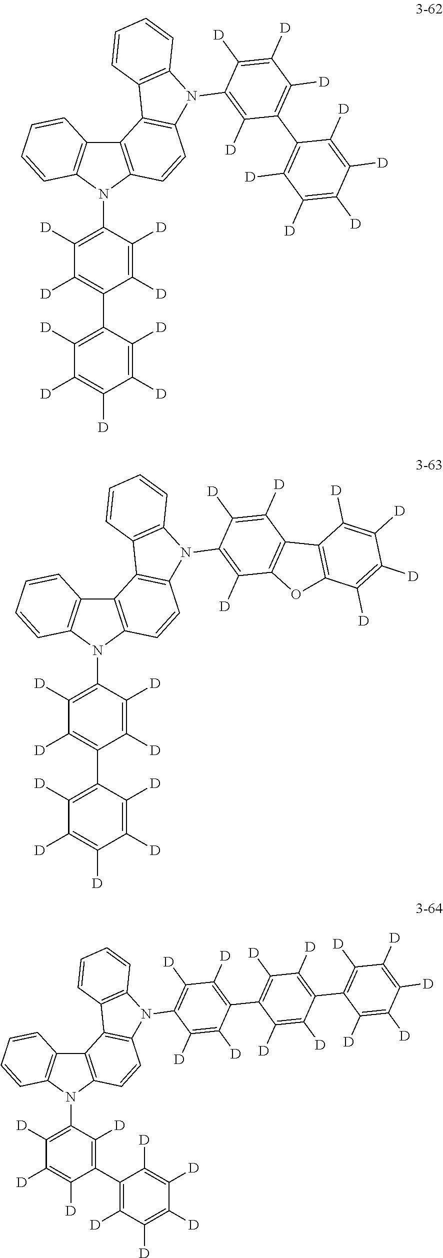

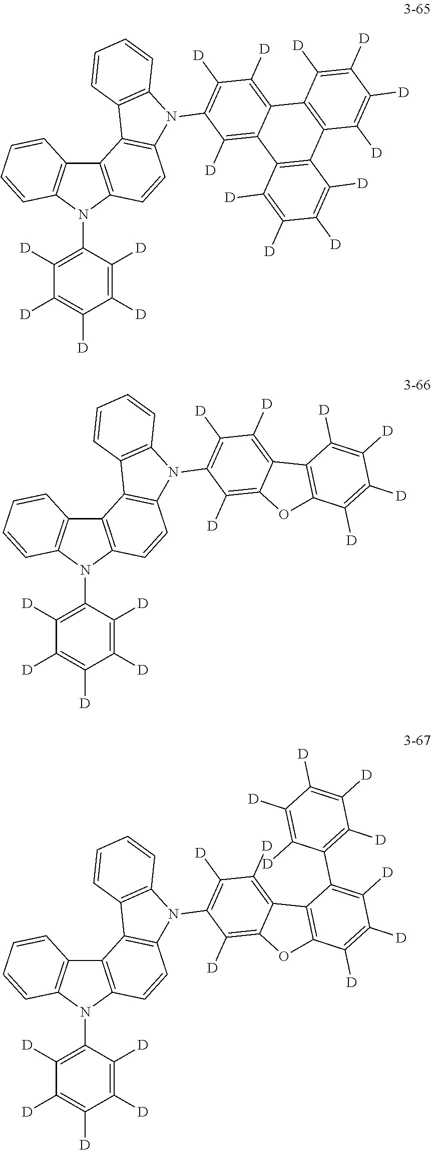 [SMILES: [2H]c1c([2H])c([2H])c(-n2c3ccccc3c3c4c5ccccc5n(-c5c([2H])c([2H])c6c7c([2H])c([2H])c([2H])c([2H])c7c7c([2H])c([2H])c([2H])c([2H])c7c6c5[2H])c4ccc32)c([2H])c1[2H] ; [2H]c1c([2H])c([2H])c(-n2c3ccccc3c3c4c5ccccc5n(-c5c([2H])c([2H])c6c(oc7c([2H])c([2H])c([2H])c([2H])c76)c5[2H])c4ccc32)c([2H])c1[2H] ; [2H]c1c([2H])c([2H])c(-c2c([2H])c([2H])c([2H])c3oc4c([2H])c(-n5c6ccccc6c6c7c8ccccc8n(-c8c([2H])c([2H])c([2H])c([2H])c8[2H])c7ccc65)c([2H])c([2H])c4c23)c([2H])c1[2H]]

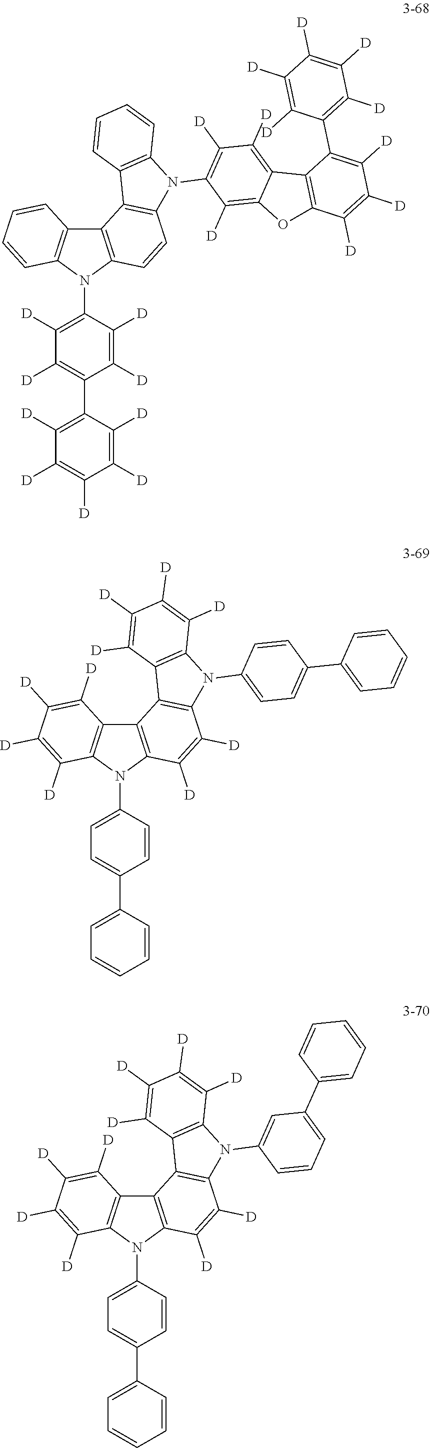 [SMILES: [2H]c1c([2H])c([2H])c(-c2c([2H])c([2H])c(-n3c4ccccc4c4c5c6ccccc6n(-c6c([2H])c([2H])c7c(oc8c([2H])c([2H])c([2H])c(-c9c([2H])c([2H])c([2H])c([2H])c9[2H])c87)c6[2H])c5ccc43)c([2H])c2[2H])c([2H])c1[2H] ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1 ; [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1ccc(-c2ccccc2)cc1]

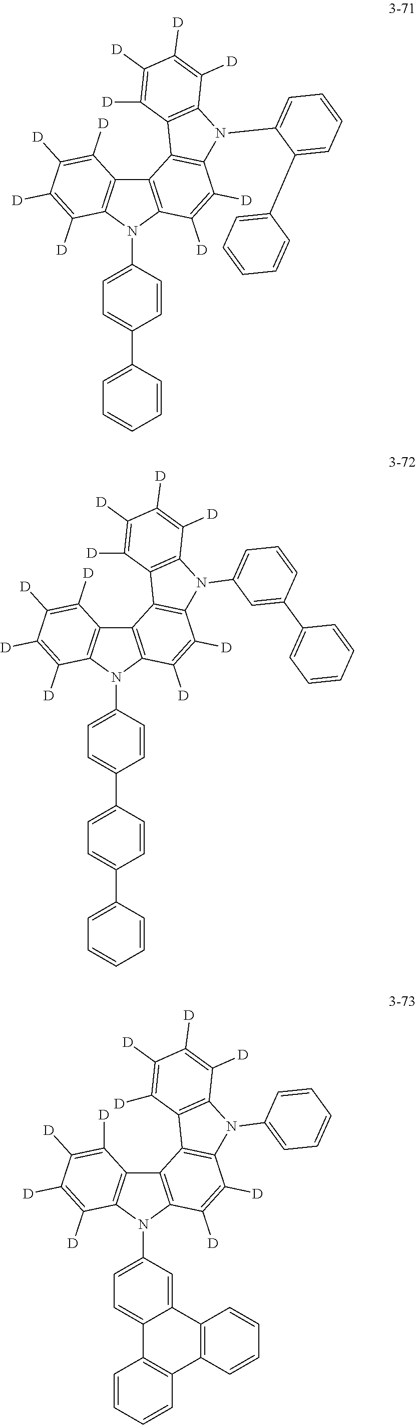 [SMILES: [2H]c1c([2H])c([2H])c2c(c1[2H])c1c3c4c([2H])c([2H])c([2H])c([2H])c4n(-c4ccccc4-c4ccccc4)c3c([2H])c([2H])c1n2-c1ccc(-c2ccccc2)cc1 ; [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1ccc(-c2ccc(-c3ccccc3)cc2)cc1 ; [2H]c1c([2H])c([2H])c2c(c1[2H])c1c3c4c([2H])c([2H])c([2H])c([2H])c4n(-c4ccc5c6ccccc6c6ccccc6c5c4)c3c([2H])c([2H])c1n2-c1ccccc1]

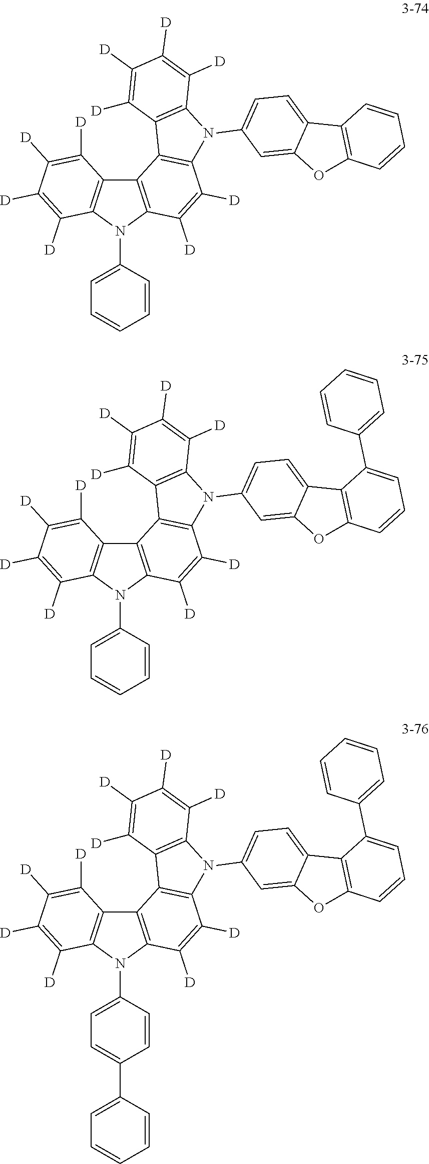 [SMILES: [2H]c1c([2H])c([2H])c2c(c1[2H])c1c3c4c([2H])c([2H])c([2H])c([2H])c4n(-c4ccc5c(c4)oc4ccccc45)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4cccc(-c6ccccc6)c45)c3c([2H])c([2H])c1n2-c1ccccc1 ; [2H]c1c([2H])c([2H])c2c(c1[2H])c1c3c4c([2H])c([2H])c([2H])c([2H])c4n(-c4ccc5c(c4)oc4cccc(-c6ccccc6)c45)c3c([2H])c([2H])c1n2-c1ccc(-c2ccccc2)cc1]

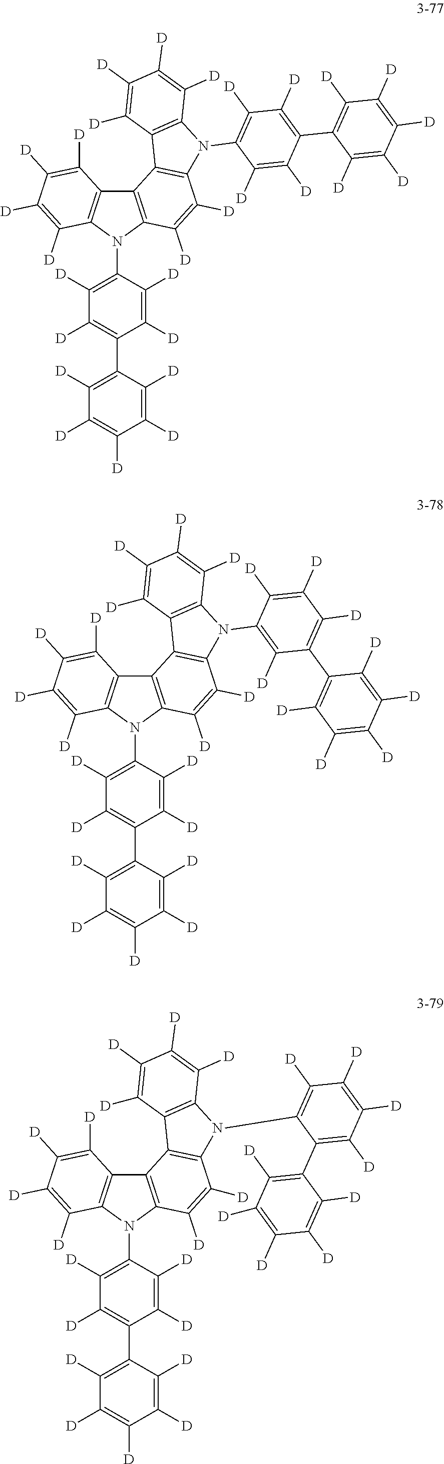

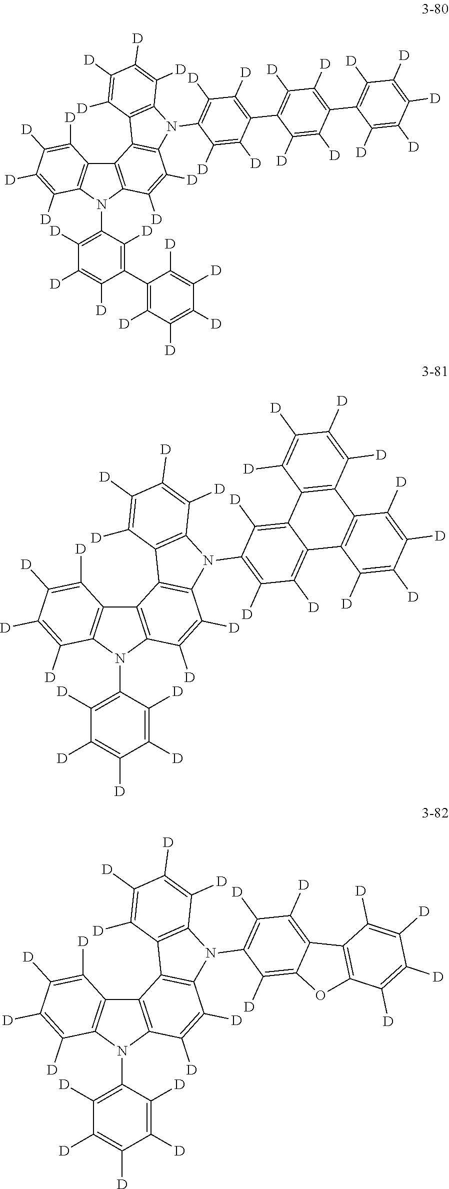

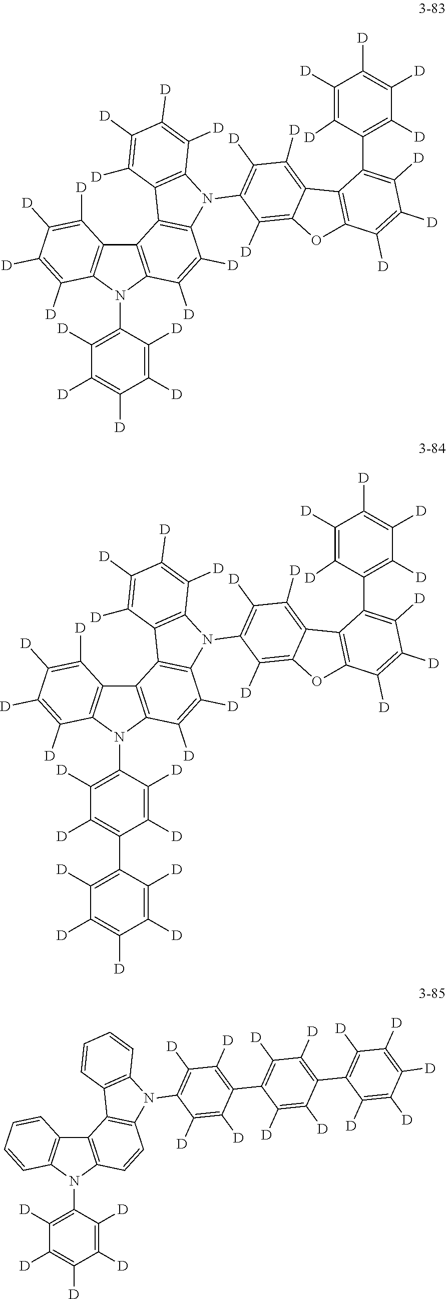

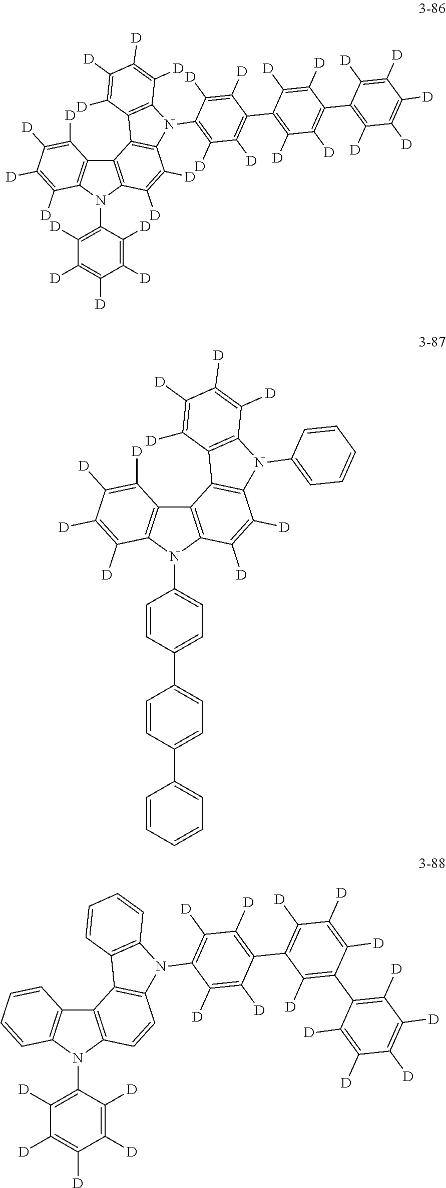

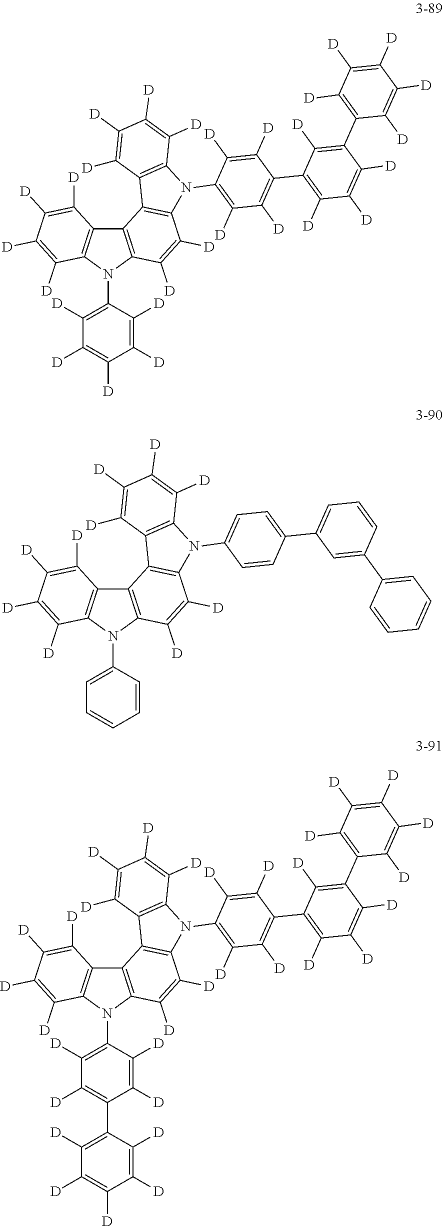

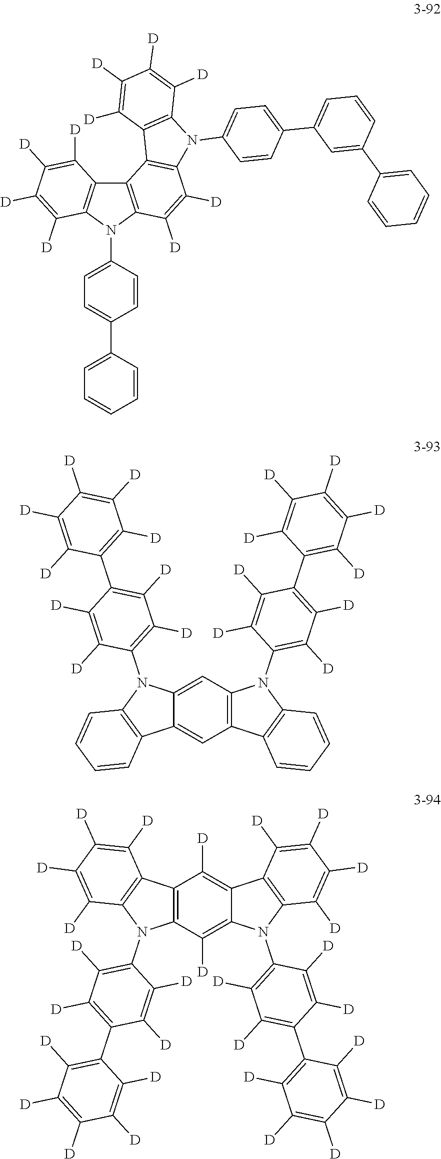

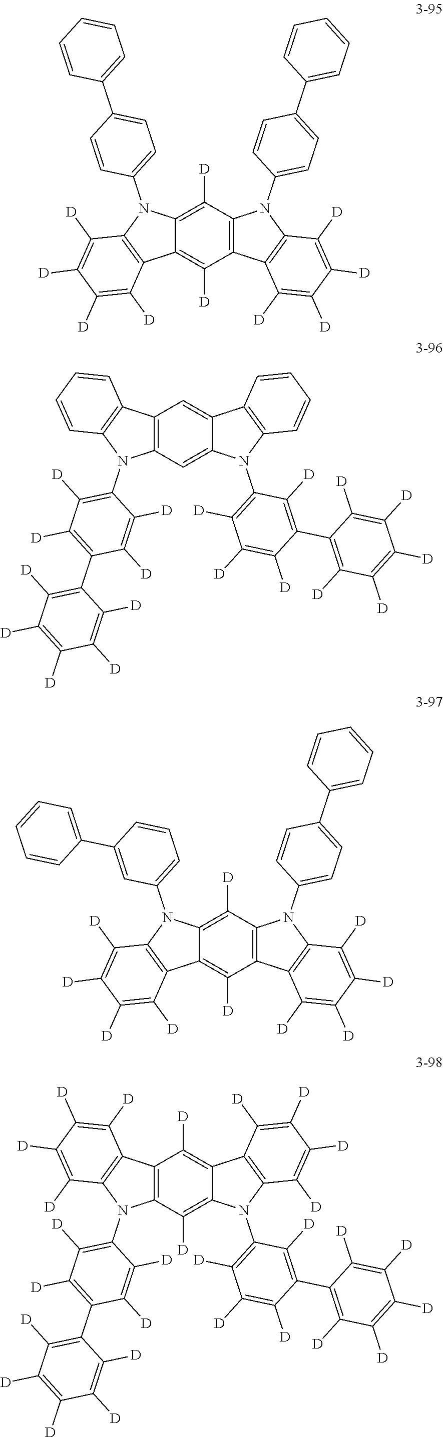

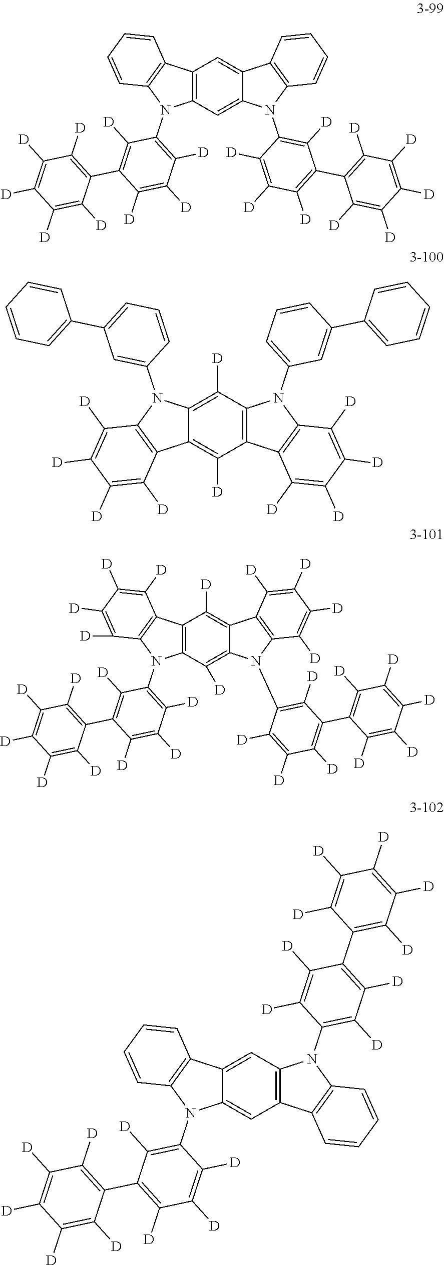

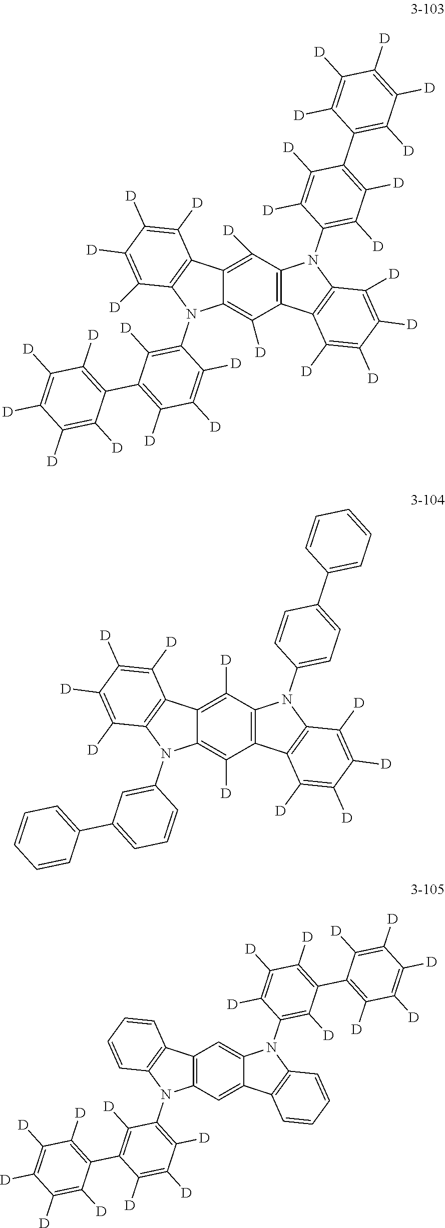

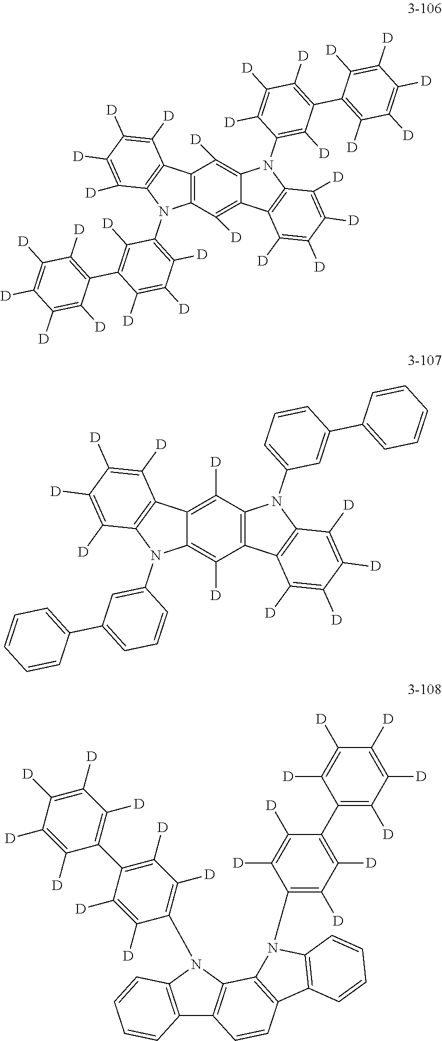

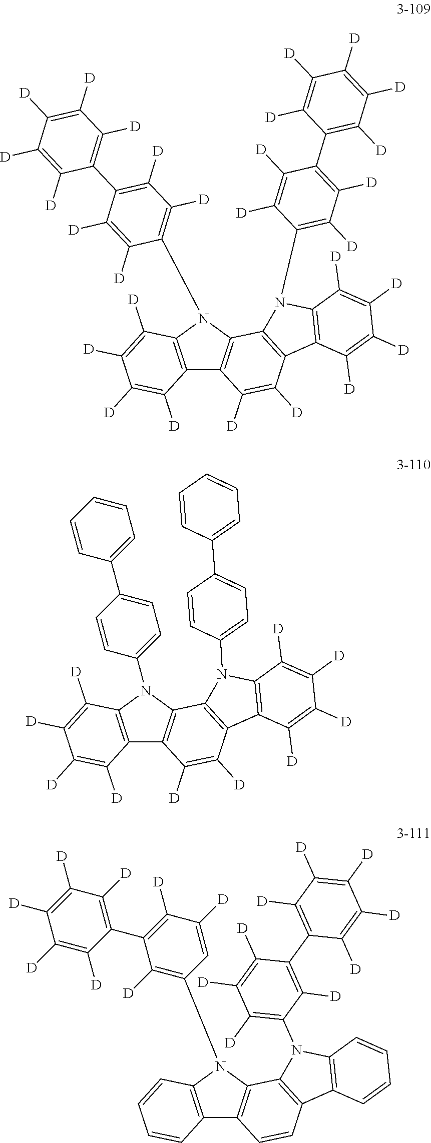

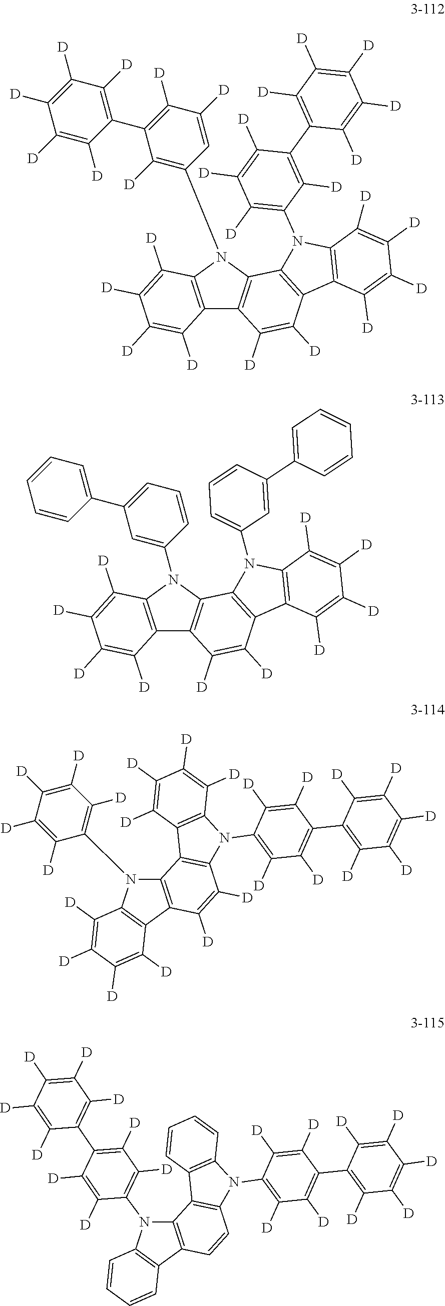

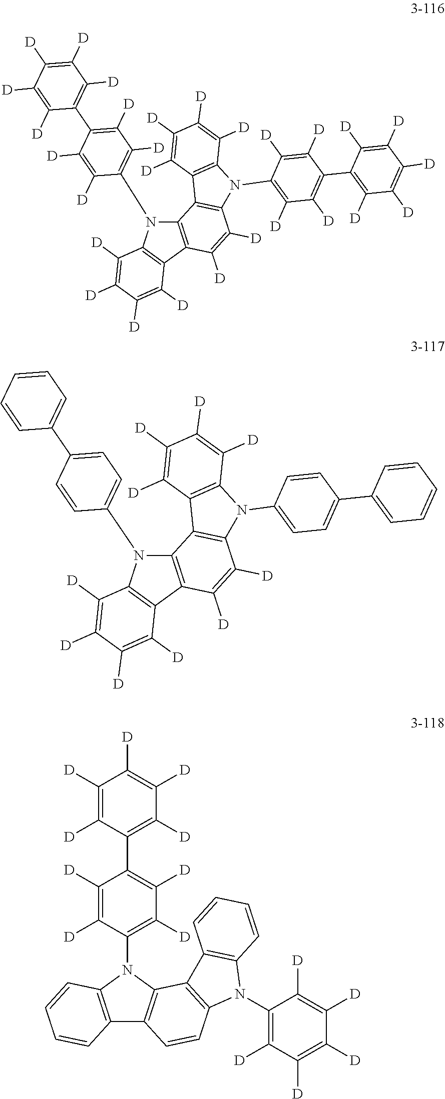

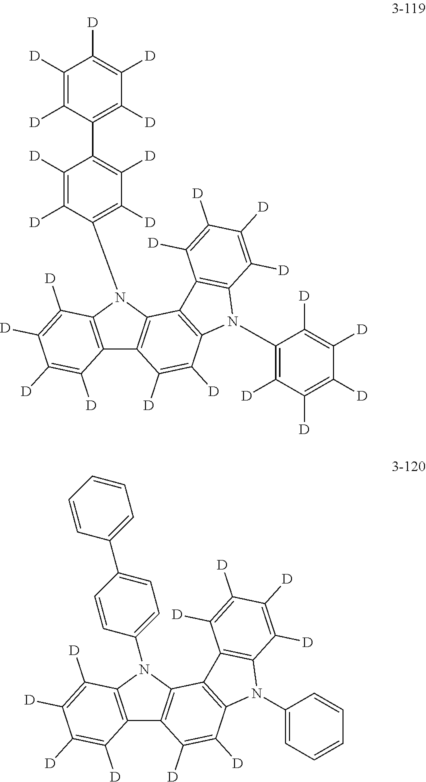 [SMILES: [2H]c1c([2H])c([2H])c(-c2c([2H])c([2H])c(-n3c4c([2H])c([2H])c([2H])c([2H])c4c4c([2H])c([2H])c5c(c6c([2H])c([2H])c([2H])c([2H])c6n5-c5c([2H])c([2H])c([2H])c([2H])c5[2H])c43)c([2H])c2[2H])c([2H])c1[2H] ; [2H]c1c([2H])c([2H])c2c(c1[2H])c1c(c([2H])c([2H])c3c4c([2H])c([2H])c([2H])c([2H])c4n(-c4ccc(-c5ccccc5)cc4)c31)n2-c1ccccc1]

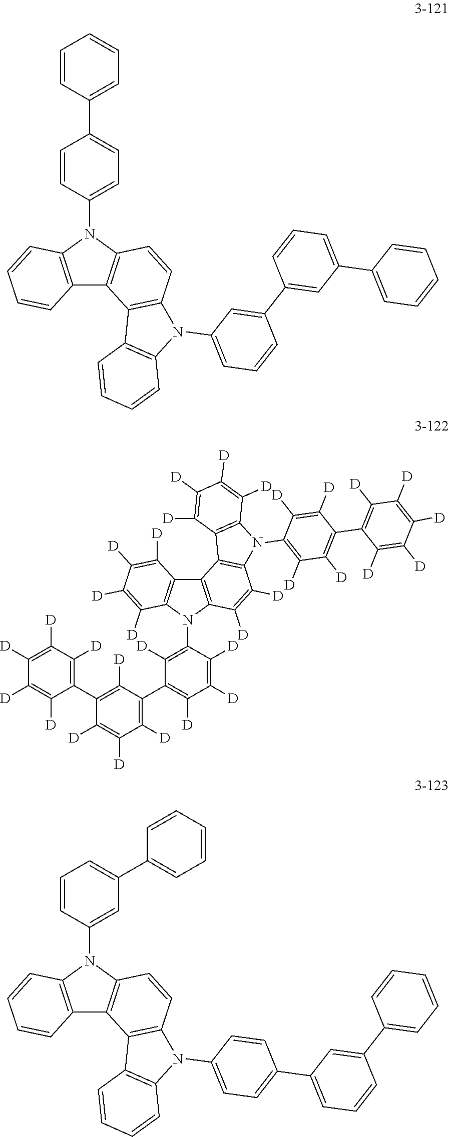 [SMILES: c1ccc(-c2ccc(-n3c4ccccc4c4c5c6ccccc6n(-c6cccc(-c7cccc(-c8ccccc8)c7)c6)c5ccc43)cc2)cc1 ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c(-c8c([2H])c([2H])c([2H])c([2H])c8[2H])c7[2H])c6[2H])c5c([2H])c([2H])c43)c([2H])c2[2H])c([2H])c1[2H] ; c1ccc(-c2cccc(-c3ccc(-n4c5ccccc5c5c6c7ccccc7n(-c7cccc(-c8ccccc8)c7)c6ccc54)cc3)c2)cc1]

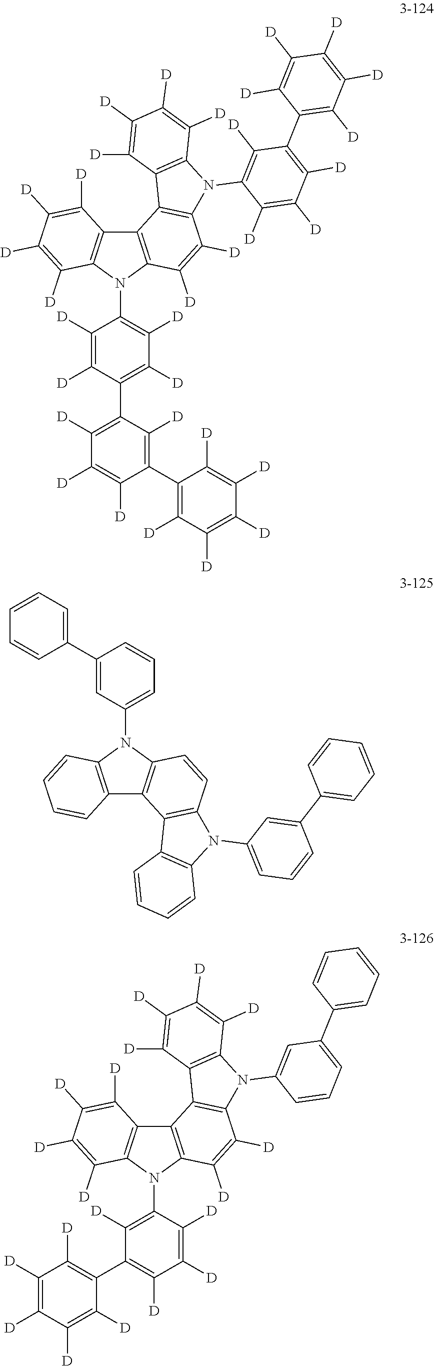

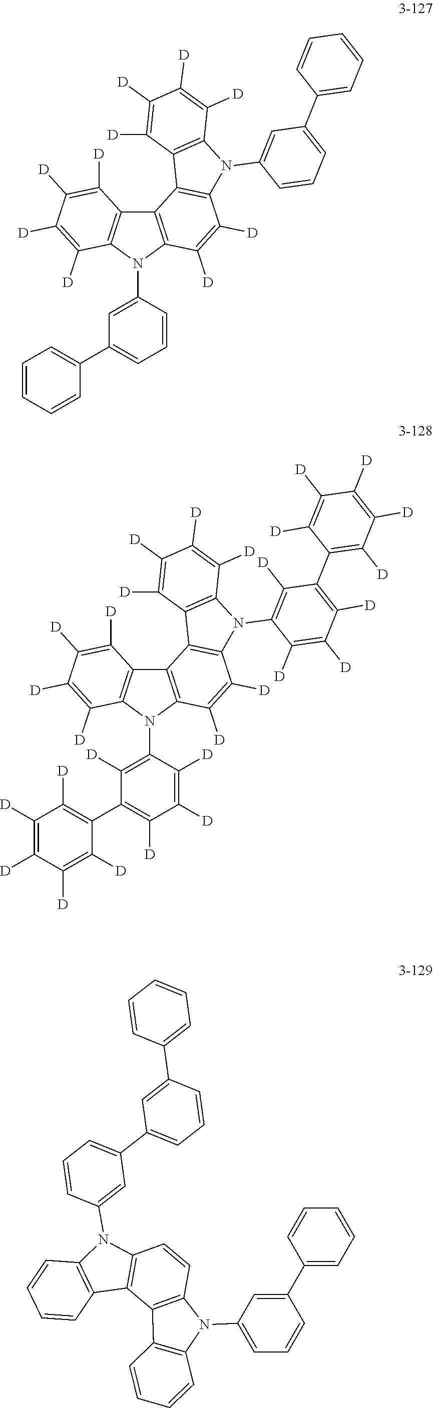 [SMILES: [2H]c1c([2H])c([2H])c2c(c1[2H])c1c3c4c([2H])c([2H])c([2H])c([2H])c4n(-c4cccc(-c5ccccc5)c4)c3c([2H])c([2H])c1n2-c1cccc(-c2ccccc2)c1 ; [2H]c1c([2H])c([2H])c(-c2c([2H])c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c2[2H])c([2H])c1[2H] ; c1ccc(-c2cccc(-c3cccc(-n4c5ccccc5c5c6c7ccccc7n(-c7cccc(-c8ccccc8)c7)c6ccc54)c3)c2)cc1]

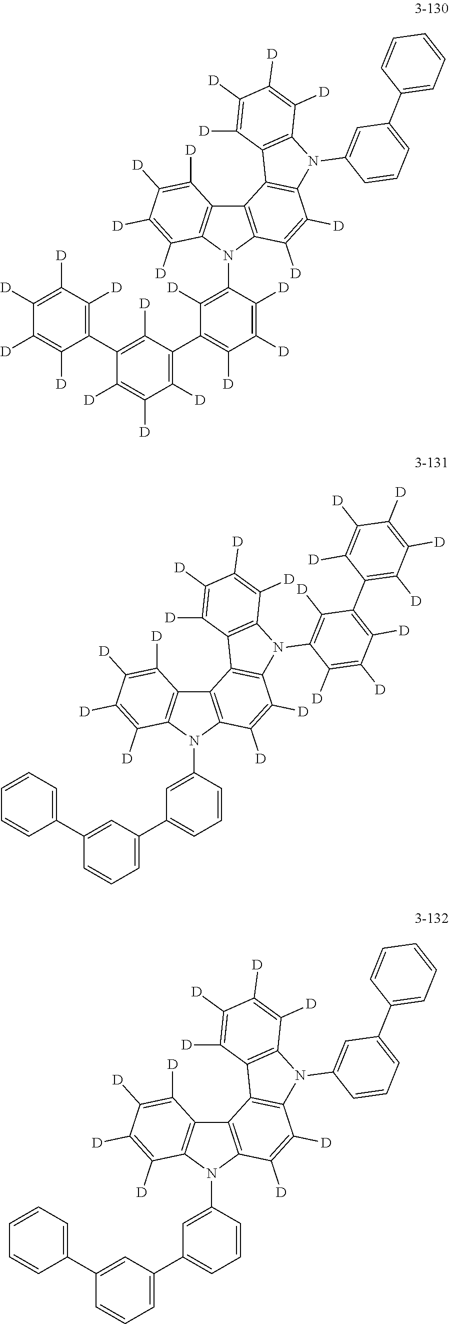

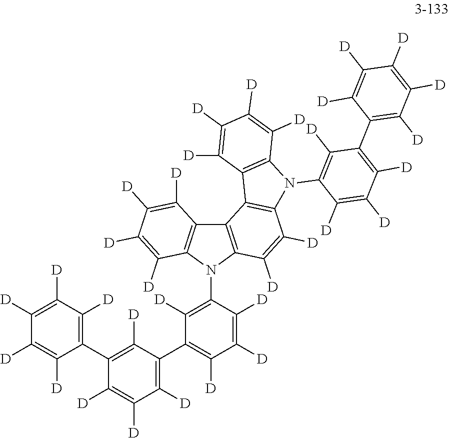 [SMILES: [2H]c1c([2H])c([2H])c(-c2c([2H])c([2H])c([2H])c(-c3c([2H])c([2H])c([2H])c(-n4c5c([2H])c([2H])c([2H])c([2H])c5c5c6c7c([2H])c([2H])c([2H])c([2H])c7n(-c7c([2H])c([2H])c([2H])c(-c8c([2H])c([2H])c([2H])c([2H])c8[2H])c7[2H])c6c([2H])c([2H])c54)c3[2H])c2[2H])c([2H])c1[2H]]

13. The organic light-emitting device according to claim 10, wherein a weight ratio of the compound represented by Chemical Formula 1 and the compound represented by Chemical Formula 3 is 1:1 to 1:10, or a weight ratio of the compound represented by Chemical Formula 1 and the compound represented by Chemical Formula 4 is 1:1 to 1:10.
